(19) United States
(12) Patent Application Publication
(10) Pub. No.: US 2013/0136691 A1
(43) Pub. Date: May 30, 2013

(54) GASTRIN RELEASING PEPTIDE COMPOUNDS

(71) Applicants: Adrian D. Nunn, Lambertville, NJ (US); Laura Lantry, Lithia Springs, GA (US)

(72) Inventors: Adrian D. Nunn, Lambertville, NJ (US); Laura Lantry, Lithia Springs, GA (US)

(73) Assignee: BRACCO IMAGING S.P.A., Milan (IT)

(21) Appl. No.: 12/993,620

(22) PCT Filed: May 19, 2009
(86) PCT No.: PCT/US09/44447

§ 371 Date: Jun. 7, 2011

Related U.S. Application Data
(60) Provisional application No. 61/054,335, filed on May 19, 2008.

(51) Int. Cl. A61K 51/08

(52) U.S. Cl. CPC A61K 51/088
USPC 424/1.69

(57) ABSTRACT

Methods and compositions for diagnosing, staging disease, monitoring therapeutic effect of drugs and imaging a patient are provided, including radiopharmaceutical formulations. Compositions comprising Ga-AMBA complexed with a radioactive isotope are provided; as are methods of imaging Gastrin Releasing Peptide receptor (GRP-R) bearing tissue and methods of diagnosing or staging disease in patients suspected of having disease associated with aberrant GRP-R function. Further, methods of monitoring therapeutic effect of a drug targeted to a receptor that crosstalks with GRP-R are provided; as are methods of pre-dosing/co-dosing non-target tissues containing GRP-R. Particularly, methods of monitoring activity of receptors and receptor pathways in vivo/in vitro by using a ligand that binds to the GRP-R are provided; as are methods of measuring the activity of a receptor or group of receptors and their associated pathways that exhibit crosstalk with the GRP-R by using such a ligand which is also detectable by external means.

### CROSS REFERENCE TO RELATED APPLICATIONS

This application claims benefit of and incorporates by reference in their entirety each of the following applications: U.S. Ser. No. 61/054,335, filed Feb. 19, 2008; U.S. Ser. No. 11/352,156, filed Feb. 10, 2006, which is a continuation-in-part of U.S. Ser. No. 11/165,721, filed Jun. 24, 2005, which is a continuation-in-part of U.S. Ser. No. 10/828,925, filed Apr. 20, 2004, which is a continuation-in-part application of International Application PCT/US2003/041328, filed Dec. 24, 2003, which is a continuation-in-part application of U.S. Ser. No. 10/341,577 filed Jan. 13, 2003. All of the above applications are hereby incorporated by reference in their entirety.

### FIELD OF THE INVENTION

This invention relates to novel gastrin releasing peptide (GRP) compounds which are useful as diagnostic imaging agents or radiotherapeutic agents. These GRP compounds are labeled with radionuclides or labels detectable by in vivo light imaging and include the use of novel linkers between the label and the targeting peptide, which provides for improved pharmacokinetics.

The present invention also describes a means of monitoring the activity of drugs which target specific receptors and receptor pathways in vivo and in vitro by observing their effect on Gastrin Releasing Peptide (GRP) receptors by using a ligand that binds to the GRP receptor. More particularly the present invention provides a means of measuring the activity of a receptor or group of receptors and their associated pathways that exhibit crosstalk with the GRP receptor by the use of a ligand that binds to the GRP receptor and which can be detected by some external means. Such means include radionuclide imaging, optical imaging or other methods of imaging the distribution of ligand in vivo or in vitro.

### BACKGROUND OF THE INVENTION

The use of radiopharmaceuticals (e.g., diagnostic imaging agents, radiotherapeutic agents) to detect and treat cancer is well known. In more recent years, the discovery of site-directed radiopharmaceuticals for cancer detection and/or treatment has gained popularity and continues to grow as the medical profession better appreciates the specificity, efficacy and utility of such compounds.

Targeted therapy, in which a drug is given to interact with a specific receptor or pathway in a cell or in the matrix surrounding the cell has gained ground in recent years particularly in the field of cancer. Targeted therapy starts from the idea that a given pathology is driven by a single or limited number of mechanisms at the cellular level, but clearly the development of resistance is a manifestation of adaptability perhaps driven by heterogeneity and/or genomic instability. One of the possible reasons for failure of a targeted drug despite presence of the target is that there are alternative systems that the cells are using or can use if the targeted system is perturbed, thus allowing the cells to escape destruction (cell death), and continue to function and survive.

In the last few years about 65% of new oncology drugs have in their labeling approved by the FDA a requirement for demonstration of the target in a patient before administration of the drug that interacts with that target. Despite some individual spectacular successes, the overall results have been less than optimal. For instance, in breast cancer treated with Herceptin®, which targets HER2/neu (ERBB2; EGFR2; a member of the Epidermal Growth Factor Receptor (EGFR) Family), there is an overall 9% response (30% have HER2/neu, 30% treated respond). For metastatic colon cancer, Erbitux which targets EGFR has an 11-14% response, and Avastin® which targets VEGFR has a 10% response. One source of this variability is the known heterogeneity in phenotype and genotype both within a primary and between different metastases in the same patient.

This heterogeneity may be present soon after the development of the tumor mass as cancer cells are known to be genetically unstable, it may arise as a change in phenotype due to the particular environment that a tumor cell experiences in the body or it may arise because of the effects of prior treatment.

Most methods of determining genotype or phenotype involve taking either biopsy specimens or blood samples. Biopsies are prone to sampling error within the tumor and suffer the further limitation that multiple samples cannot easily be taken from the same tumor in a patient nor of multiple tumor sites within the body because of the invasiveness of the procedure. In addition there are some tissues, such as bone, for which it is difficult to take biopsy samples. This is particularly pertinent to breast and prostate cancers that metastasize to the bones. It is easier for multiple liquid samples such as blood or urine to be taken but these suffer the drawback that they provide only an average signal for the whole body. An imaging method in which all tissues of the body can be interrogated at once and which can be used on multiple occasions is preferable.

Whereas solid samples are normally interrogated for one or at most a small number of targets of interest, liquid samples may be interrogated for a large number of analytes, for instance by gene chip or proteomic chip assays. Whereas it might appear that the administration and imaging of a single substance may be inferior to a multiplexed array it should be noted that the use of an appropriate substance may allow interrogation of not only a target but also of the various regulatory and signaling pathways that make up the target system. In optimal circumstances the data provide a functional readout of the target and its associated systems.

The use of targeted drugs which are efficacious in a low percentage of patients despite many having the target is deleterious to those patients that do not respond, both in time wasted before a better therapy is started and because of the potential for toxic effects without benefit. It is also wasteful of health system resources. For instance, in 2007 the worldwide sales of some targeted drugs is expected to be in the $1-3 billion range. Sales of some oncology drugs in 2007 based on first quarter sales (×4) are Herceptin® $1.9B, Avastin® $2.4B, Rituxan® $2.3B and Erbitux® $1.2B. If the majority of this material is used in patients who do not respond it represents an enormous financial drain and is detrimental in lost time or potential adverse events in those patients who do not respond. Currently solid tumor response is measured using morphologic criteria such as the WHO or RECIST criteria as a surrogate for increased survival. However, such morphologic responses take considerable time to become observable after initiation of treatment, even under the best of circumstances with optimal treatment and response, during which time inappropriate treatment of an eventual non-responder may continue.

A means of further characterizing the response of a receptor, cell or group of cells is necessary. In recent years there has been an increased use of imaging to characterize tumor response at the biochemical level either before a morphological response is detectable or if there is no anticipated change in the morphology of the tumor.

The most common method of performing such biochemical imaging is to use F-18 Fluorodeoxyglucose which reports on part of the glycolytic pathway (e.g. glucose metabolism). Although some promising results have been obtained, it suffers from reduced specificity because uptake is also increased in areas of inflammation which can occur with or independently of tumors and which can rise and fall independently of tumor glycolysis. Indeed, glycolysis is also the major form of energy production by most normal tissues. Thus normal tissues may present an increased background which may make it difficult to isolate the signal generated by the tumour tissue. In addition areas of inflammation also exhibit increased glucose consumption which is a particular problem with some inflammatory cancers such as breast cancer. Inflammation may be confused with cancer. Additionally, inflammation, stimulated as a normal response to the treatment of a tumour, may be misinterpreted as an increase in tumour glycolysis, and thus a failure of treatment. In addition glycolysis is one of the basal systems in most cells and so is far downstream of most drug targets. Finally, FDG is cyclotron produced which can limit its availability.

Two other methods of a general nature are those that use F-18 Fluorothymidine to measure cell proliferation and F-18 Fluorocholine to measure aspects of lipid turnover. Each of these techniques is quite sensitive but because each measures some aspect of metabolism that is also used by normal cells they suffer from reduced specificity

What is needed is an agent that provides and indication of the effect of a drug on a target which is upstream of the basal level of energy production, closer to the drug's target. Ideally, what is needed is an agent that provides information from a point in the various pathways that shows sensitivity to the effects of a variety of drug/target interactions.

One method is to directly measure the target in question. For instance Herceptin® has been radiolabeled and the distribution in patients measured prior to and after treatment with Herceptin®. The results show that it is not possible to predict cardiotoxicity based on the myocardial uptake levels, that only some of the known tumors were visualized, and that additional tumors can be identified. Prediction of response to treatment was not attempted. On the other hand response to treatment in an animal system has been shown using a radiolabeled F(ab)₂ fragment of Herceptin®.

In vitro testing for another member of the EGFR family, EGFR is employed in many clinical trials to select patients for EGFR targeted therapies, and it is required by the FDA for selection of patients for treatment with Erbitux®. Its utility as a predictor of response is not yet clear. Most EGFR studies have not produced data that indicates which subsets of patients may derive the most benefit from EGFR-targeted agents. EGFR is over expressed to varying degrees in a wide variety of malignancies. Many new tests for EGFR expression and for mutations are in development and a large number of clinical trials are underway to elucidate their roles in determining patient selection for EGFR-targeted therapies.

EGFR expression has been examined in vivo both with radiolabeled derivatives of the antibody used to treat cancers expressing this target (Cetuximab®/C225) and with the known ligand, EGF, or small molecule inhibitors. Although in some cases there was a good correlation between uptake of radioactivity in the tumor tissue and presence of EGFR measured by independent means, no attempt to predict response was made. Indeed, blocking studies (with drug) to demonstrate specificity highlight a general problem of using ligands for the target to image the target involved in therapy, in that administration of the targeted therapeutic to the animals blocked uptake of the imaging ligand by the target. This can result in diminished or absent signal with no relationship to response unless there is a single and direct relationship between target occupancy and response. That this may not be so is evident from the low responses of patients despite known presence of target.

Another drawback to using specific ligands for each receptor or target is that there are numerous targeted drugs to different targets already approved for routine use with many more in clinical trials. The potentially large number of ligands required would tax the development and approval process. In addition, given that there may be numerous targeted drugs, each with good response, but only in a small subset of patients, one can anticipate that various combinations may be beneficial which again, under the one target one imaging agent scenario, would require multiple procedures to evaluate the potential for response.

Finally, the known presence of mutations in many of the targets suggests that presence of target is insufficient and knowledge of blocking of function is required.

Thus there is a need to develop a more general method of determining the activity of targeted drugs on the functioning of their target, preferably a means that can report on the activity of more than one target/drug interaction and one that is specific for pathologies showing minimal interference from normal tissue.

It is known that many of the receptors addressed by targeted drugs exhibit “crosstalk”. “Crosstalk” refers to the situation in which modulation of the activity of one receptor influences the expression level or activity of other, not directly connected, receptors. Such crosstalk is not of the type characterized by the interaction of the closely related tyrosine kinases EGFR and HER2, which form dimers when the cognate ligand binds, but is of the type where receptors of different classes can interact through intra- or extracellular pathways. For instance, it is known that the expression level of the somatostatin receptor can be modulated by the occupancy and activity of the estrogen receptor. In this case the somatostatin receptor is a G protein coupled receptor (GPCR) normally residing on the external cell membrane and from which signal initiation originates and the estrogen receptor normally resides close to the nucleus and has activity within the nucleus. Estrogen acts through a nuclear hormone receptor that upon activation increases transcription and expression of hormone-responsive genes.

The work shows an increase in the binding per cell of a radiolabeled somatostatin receptor binder for two human breast cancer cell lines having both estrogen and somatostatin receptors after exposure to estrogen was blocked by an antagonist or partial agonist. A similar increase did not occur in a cell line that had somatostatin receptors but no estrogen receptors. Three breast cancer patients were examined before and shortly after commencement of anti-estrogen therapy. Results were mixed with some decrease in tumor uptake in one patient.

Many of the approved drugs are directed towards GPCRs but in recent years drugs targeted towards receptor tyrosine kinases (RTK) have become more common and many of the new cancer drugs target RTKs. For instance, the following is a partial list of the RTKs known to exhibit crosstalk, and some of the approved drugs for inhibiting their action.

| RTK target | Drug |
|---|---|
| HER2 | trastuzumab/Herceptin ® |
| EGFR | cetuximab/Erbitux ® |
| EGFR | erlotinib/Tarceva ® |
| EGFR/HER2 | lapatinib/Tykerb ® |
| VEGFR2/PDGFR | sorafenib/Nexavar ® |
| PDGFR | sunitinib/Sutent ® |
| EGFR | gefitinib/Iressa ® |
| PDGFR/KIT | imatinib/Gleevec ® |
| VEGF/VEGFR2 | bevacizumab/Avastin ® |

Due to their mode of action drugs targeted towards RTKs are not expected to have any direct action on GPCRs.

One class of receptors that are not widely distributed in normal adult tissue outside of the brain and which are known to exhibit crosstalk with a variety of other receptors is the GRP receptor family. There are three known subtypes of this receptor in humans, BB1 (NMBR), BB2 (GRPR), bb3 (BRS-3) with a fourth amphibian receptor, bb4, identified by sequence similarity. Crosstalk between the GRP receptor family and a number of RTKs has been demonstrated independently. For instance GRP has been shown to increase activation of EGFR via stimulation of TNF-α converting enzyme and release of amphiregulin, one of the precursor ligands for EGFR. Administration of a GRP receptor antagonist causes down regulation of the number of EGF receptors.

In these, and many other examples the crosstalk is in the GRPR to RTK (e.g. EGFR) direction, i.e. application of a GRPR agonist/antagonist causes activation/deactivation of EGFR signaling. No data are available documenting crosstalk in the opposite direction, RTK to GRPR, which might result in increased or decreased expression of the GRP receptor specific signal which may be detectable by in vivo imaging.

### GRP-Receptor Targeted Radiopharmaceuticals

Several targeted radiopharmaceuticals designed to localize in cancerous tissue have recently been reported. These newer radiopharmaceutical agents typically consist of a targeting agent connected to a metal chelator, which can be chelated to (e.g., complexed with) a diagnostic metal radionuclide such as, for example, technetium, indium, or gallium or a therapeutic metal radionuclide such as, for example, lutetium, yttrium, or rhenium. The role of the metal chelator is to hold (i.e., chelate) the metal radionuclide as the radiopharmaceutical agent is delivered to the desired site. A metal chelator which does not bind strongly to the metal radionuclide would render the radiopharmaceutical agent ineffective for its desired use since the metal radionuclide would therefore not reach its desired site. Thus, further research and development led to the discovery of metal chelators, such as those reported in U.S. Pat. No. 5,662,885 to Pollak et al and U.S. Pat. No. 6,13,274 to Tweedle et al., hereby incorporated by reference, which exhibited strong binding affinity for metal radionuclides and the ability to conjugate with the targeting agent. Subsequently, the concept of using a “spacer” to create a physical separation between the metal chelator and the targeting agent was further introduced, for example in U.S. Pat. No. 5,976,495 to Pollak et. al., hereby incorporated by reference.

The role of the targeting agent, by virtue of its affinity for certain binding sites in the body, is to direct the diagnostic agent, such as a radiopharmaceutical agent containing the metal radionuclide, to the desired site for detection or treatment. Typically, the targeting agent may include a protein, a peptide, or other macromolecule or a small molecule which exhibits a specific affinity for a given receptor. Other known targeting agents include monoclonal antibodies (MAbs), antibody fragments (F_{ab}'s and (F_{ab})₂'s), and receptor-avid peptides. Donald J. Buchsbaum, “Cancer Therapy with Radiolabeled Antibodies; Pharmacokinetics of Antibodies and Their Radiolabels; Experimental Radioimmunotherapy and Methods to Increase Therapeutic Efficacy,” CRC Press, Boca Raton, Chapter 10, pp. 115-140, (1995); Fischman, et al. “A Ticket to Ride: Peptide Radiopharmaceuticals,” The Journal of Nuclear Medicine, vol. 34, No. 12, (December 1993). These references are hereby incorporated by reference in their entirety.

In recent years, it has been learned that some cancer cells contain gastrin releasing peptide (GRP) receptors (GRP-R) of which there are a number of subtypes. In particular, it has been shown that several types of cancer cells have over-expressed or uniquely expressed GRP receptors. For this reason, much research and study has been done on GRP and GRP analogues which bind to the GRP receptor family. One such analogue is bombesin (BBN), a 14 amino acid peptide (i.e., tetradecapeptide) isolated from frog skin which is an analogue of human GRP and which binds to GRP receptors with high specificity and with an affinity similar to GRP.

Bombesin and GRP analogues may take the form of agonists or antagonists. Binding of GRP or BBN agonists to the GRP receptor increases the rate of cell division of these cancer cells and such agonists are internalized by the cell, while binding of GRP or BBN antagonists generally does not result in either internalization by the cell or increased rates of cell division. Such antagonists are designed to competitively inhibit endogenous GRP binding to GRP receptors and reduce the rate of cancer cell proliferation. See, e.g., Hoffken, K.; Peptides in Oncology II, Somatostatin Analogues and Bombesin Antagonists (1993), pp. 87-112. For this reason, a great deal of work has been, and is being pursued to develop BBN or GRP analogues that are antagonists. E.g., Davis et al., Metabolic Stability and Tumor Inhibition of Bombesin/GRP Receptor Antagonists, Peptides, vol. 13, pp. 401-407, 1992.

In designing an effective compound for use as a diagnostic or therapeutic agent for cancer, it is important that the drug have appropriate in vivo targeting and pharmacokinetic properties. For example, it is preferable that for a radiopharmaceutical, the radiolabeled peptide have high specific uptake by the cancer cells (e.g., via GRP receptors). In addition, it is also preferred that once the radionuclide localizes at a cancer site, it remains there for a desired amount of time to allow imaging or, for therapeutic purposes, to deliver a highly localized radiation dose to the site.

Moreover, developing radiolabeled peptides that are cleared efficiently from normal tissues is also an important factor for radiopharmaceutical agents. When biomolecules (e.g., MAb, F_{ab} or peptides) labeled with metallic radionuclides (via a chelate conjugation), are administered to an animal such as a human, a large percentage of the metallic radionuclide (in some chemical form) can become “trapped” in either the kidney or liver parenchyma (i.e., is not excreted into the urine or bile). Duncan et al.; Indium-1,1-Diethylenetriaminepentaacetic Acid-Octreotide Is Delivered in Vivo to Pancreatic, Tumor Cell, Renal, and Hepatocyte Lysosomes, Cancer Research 57, pp. 659-671, (Feb. 15, 1997). For the smaller radiolabeled biomolecules (i.e., peptides or F_{ab}), the major route of clearance of activity is through the kidneys which can also retain high levels of the radioactive metal (i.e., normally >10-15% of the injected dose). Retention of metal radionuclides in the kidney or liver is clearly undesirable. Conversely, clearance of the radiopharmaceutical from the blood stream too quickly by the kidney is also undesirable if longer input for diagnostic imaging or high tumor uptake for radiotherapy is needed.

Subsequent work, such as that in U.S. Pat. No. 6,200,546 and US 2002/0054855 to Hoffman, et. al, hereby incorporated by reference in their entirety, have attempted to overcome this problem by forming a compound having the general formula X—Y—B wherein X is a group capable of complexing a metal, Y is a covalent bond on a spacer group and B is a bombesin agonist binding moiety. Such compounds were reported to have high binding affinities to GRP receptors, and the radioactivity was retained inside the cells for extended time periods. In addition, in vivo studies in normal mice have shown that retention of the radioactive metal in the kidneys was lower than that known in the art, with the majority of the radioactivity excreted into the urine.

There remains a need for compounds which can target the GRP-R and its subtypes with improved specificity and which are useful to deliver diagnostic moieties (e.g. detectable labels) and/or therapeutic moieties.

### SUMMARY OF THE INVENTION

In an embodiment of the present invention, there is provided new and improved compounds for use in diagnostic imaging or radiotherapy. The compounds include a chemical moiety capable of complexing a medically useful metal ion or radionuclide (metal chelator) attached to a GRP receptor targeting peptide by a linker or spacer group. In another embodiment, these compounds include an optical label (e.g. a photolabel or other label detectable by light imaging, optoacoustical imaging or photoluminescence) attached to a GRP receptor targeting peptide by a linker or spacer group.

In general, compounds of the present invention may have the formula:

M-N—O—P-G

wherein M is a metal chelator (in the form complexed with a metal radionuclide or not), or a moiety that contains a radiolabeled halogen such as ¹⁸F, ¹²³I—, ¹²⁴I— or ¹³¹I— or an optical label, N—O—P is the linker, and G is the GRP receptor targeting peptide.

The metal chelator M may be any of the metal chelators known in the art for complexing with a medically useful metal ion or radionuclide. Preferred chelators include DTPA, DOTA, DO3A, HP-DO3A, EDTA, TETA, EHPG, HBED, NOTA, DOTMA, TETMA, PDTA, TTHA, LICAM, MECAM, Aazta and derivatives thereof or peptide chelators, such as, for example, those discussed herein. The metal chelator may or may not be complexed with a metal radionuclide, and may include an optional spacer such as a single amino acid.

Preferred metal radionuclides for radioimaging or radiotherapy include ^{99m}Tc, ⁵¹Cr, ⁶⁷Ga, ⁶⁸Ga, ⁴⁷Sc, ⁵¹Cr, ¹⁶⁷Tm, ¹⁴¹Ce, ¹¹¹In, ¹⁶⁸Yb, ¹⁷⁵Yb, ¹⁴⁰La, ⁹⁰Y, ⁸⁸Y, ¹⁵³Sm, ¹⁶⁶Ho, ¹⁶⁵Dy ¹⁶⁶Dy, ⁶²Cu, ⁶⁴Cu, ⁶⁷Cu, ⁹⁷Ru, ¹⁰³Ru, ¹⁸⁶Re, ⁸⁸Re, ²⁰³Pb, ²¹¹Bi, ²¹²Bi, ²¹³Bi, ²¹⁴Bi, ²²⁵Ac, ¹⁰⁵Rh ¹⁰⁹Pd, ^{117m}Sn, ¹⁴⁹Pm, ¹⁶¹Tb, ¹⁷⁷Lu, ¹⁹⁸Au and ¹⁹⁹Au. The choice of metal will be determined based on the desired therapeutic or diagnostic application. For example, for diagnostic purposes the preferred radionuclides include ⁶⁴Cu, ⁶⁷Ga, ⁶⁸Ga, ^{99m}Tc, and ¹¹¹In, with ^{99m}Tc, ¹¹¹In and ⁶⁸Ga being particularly preferred. For therapeutic purposes, the preferred radionuclides include ⁶⁴Cu, Y, ⁹⁰Y, ¹⁰⁵Rh, ¹¹¹In, ^{117m}Sn, ¹⁴⁹Pm, ¹⁵³Sm, ¹⁶¹Tb, ¹⁶⁶Dy, ¹⁶⁶Ho, ¹⁷⁵Yb, ¹⁷⁷Lu, ^{186/188}Re, and ¹⁹⁹Au, with ¹⁷⁷Lu and ⁹⁰Y being particularly preferred. A preferred chelator used in compounds of the invention is 1-substituted 4,7,10-tricarboxymethyl 1,4,7,10 tetraazacyclododecane triacetic acid (DO3A).

The moiety that contains a radiolabeled halogen such as ¹⁸F, ¹²³I—, ¹²⁴I— or ¹³¹I— is preferably one of those described in Zhang et al, J. Nuclear Med. 47:492-501 (2006), incorporated herein by reference in its entirety.

The optical label M may be any of various optical labels known in the art. Preferred labels include, without limitation, optical dyes, including organic chromophores or fluorophores, such as cyanine dyes, light absorbing compounds, light reflecting and scattering compounds, and bioluminescent molecules.

In one embodiment, the linker N—O—P contains at least one non-alpha amino acid.

In another embodiment, the linker N—O—P contains at least one substituted bile acid.

In yet another embodiment, the linker N—O—P contains at least one non-alpha amino acid with a cyclic group.

In the most preferred embodiment, M is a metal chelator and the linker N—O—P contains at least one non-alpha amino acid with a cyclic group. Additionally, M may be complexed with a radioactive or paramagnetic metal. In a more preferred embodiment M-N—O—P-G is L70 or AMBA as described in herein. In an especially preferred embodiment L70 or AMBA is complexed with ¹⁷⁷Lu (¹⁷⁷Lu-AMBA or ¹⁷⁷Lu-L70) or with a radionuclide detectable by positron emission tomography (PET), such as ⁶⁸Ga (⁶⁸Ga-AMBA or ⁶⁸Ga-L70).

The GRP receptor targeting peptide may be GRP, bombesin or any derivatives or analogues thereof. In a preferred embodiment, the GRP receptor targeting peptide is a GRP or bombesin analogue which acts as an agonist. In a particularly preferred embodiment, the GRP receptor targeting peptide is a bombesin agonist binding moiety disclosed in U.S. Pat. No. 6,200,546 and US 2002/0054855, incorporated herein by reference.

There is also provided a novel method of imaging using the compounds of the present invention. In a preferred embodiment, PET is used to image GRP-R in tissue, particularly cancerous tissue bearing the same. In a more preferred embodiment, ⁶⁸Ga-AMBA or ⁶⁸Ga-L70 is used to image GRP-R in human tissue in vivo.

A single or multi-vial kit that contains all of the components needed to prepare the diagnostic or therapeutic agents of the invention is provided in an exemplary embodiment of the present invention.

There is further provided a novel method for preparing a diagnostic imaging agent comprising the step of adding to an injectable imaging medium a substance containing the compounds of the present invention.

A novel method of radiotherapy using the compounds of the invention is also provided for the treatment or delaying the progression of pathology involving overexpression of GRP receptors, such as cancers, including, for example breast and prostate cancers. A novel method for preparing a radiotherapeutic agent comprising the step of adding to an injectable therapeutic medium a substance comprising a compound of the invention is also provided. In a preferred embodiment ¹⁷⁷Lu-AMBA or ¹⁷⁷Lu-L70 is used in such methods.

Improved methods of administration of labeled compounds of the invention are provided, as are methods of increasing targeting of GRP-expressing target tissue by labeled compounds of the invention.

The instant inventors have unexpectedly found that modulation of the activity of some RTKs or the estrogen receptor by their ligands or antagonists (including for example, cancer drugs targeted to such receptors), affects the activity of GPCRs and in particular the activity of the GRP family of receptors. Thus the detection of such activity by imaging the GRP receptors (or other methods of detecting changes in the GRP receptor) allows the indirect assessment of the activity of the RTKs or estrogen receptor as influenced by therapeutic interventions directed towards these RTKs or the estrogen receptor. Thus, the present invention provides a method of examining crosstalk with GRP receptors, specifically cross talk in the RTK or “other target” to GRPR direction, for a broad spectrum of solid human tumors that express GRPR (primaries and metastases) including, but not limited to breast and prostate cancer. Specifically, assessing the effect that treatment with any one of a broad class of therapeutics targeted to RTK receptors or the estrogen receptor (e.g. RTK inhibitors, estrogen inhibitors), administered under normal clinical conditions (dose and schedule), may have on the function of such receptors (e.g. RTK receptors or the estrogen receptor) as detected by changes in the expression of the GRP receptor specific signal with which they exhibit crosstalk. The invention provides a functional indication of the anticipated response to therapy via an increase, decrease, or no change in the GRP receptor specific signal activity in vivo. In a preferred embodiment a GRP-R targeted compound of the invention complexed with a diagnostic radionuclide detectable by, e.g. SPECT or PET is used to monitor the change in the GRP receptor (or M is a moiety comprising a radioactive halogen, which may be detected to monitor GRP-R response). In a particularly preferred embodiment ⁶⁸Ga-AMBA or ⁶⁸Ga-L70 is administered and imaged to detect the change in GRP-R signal activity in vivo.

The invention also provides a method of screening new drugs for changes in the activity of the GRP receptor family in vitro using a radiolabeled or otherwise detectable labeled agonist or antagonist of the GRP receptors. In a preferred embodiment the GRP-R targeted compound of the invention is complexed with a diagnostic radionuclide detectable by, e.g. SPECT or PET (or M is a moiety comprising a radioactive halogen, which may be detected to monitor GRP-R response). In a particularly preferred embodiment ⁶⁸Ga-AMBA or ⁶⁸Ga-L70 is administered and imaged to detect the change in GRP-R signal activity in vivo. The invention also provides a method of imaging the activity of the GRP receptor family in vivo using a radiolabeled agonist or antagonist of the GRP receptors to monitor the therapeutic effect of drugs that target a receptor that cross talks with GRP-R. In a preferred embodiment the radiolabeled GRP-R targeted compound of the invention is complexed with a diagnostic radionuclide detectable by, e.g. SPECT or PET (or M is a moiety comprising a radioactive halogen, which may be detected to monitor GRP-R response). In a particularly preferred embodiment ⁶⁸Ga-AMBA or ⁶⁸Ga-L70 is administered and imaged to detect the change in GRP-R signal activity in vivo.

### BRIEF DESCRIPTION OF THE DRAWINGS

FIG. 1A is a graphical representation of a series of chemical reactions for the synthesis of intermediate C ((3β,5β)-3-(9H-Fluoren-9-ylmethoxy)aminocholan-24-oic acid), from A (Methyl-(3β,5β)-3-aminocholan-24-ate) and B ((3β,5β)-3-aminocholan-24-oic acid), as described in Example I.

FIG. 1B is a graphical representation of the sequential reaction for the synthesis of N-[(3β,5β)-3-[[[[[4,7,10-Tris(carboxymethyl)-1,4,7,10-tetraazacyclododec-1-yl]acetyl]amino]acetyl]amino]cholan-24-yl]-L-glutaminyl-L-tryptophyl-L-alanyl-L-valyl-glycyl-L-histidyl-L-leucyl-L-methioninamide (L62), as described in Example I.

FIG. 2A is a graphical representation of the sequential reaction for the synthesis of N-[4-[[[[[4,7,10-Tris(carboxymethyl)-1,4,7,10-tetraazacyclododec-1-yl]acetyl]amino]acetyl]amino]benzoyl]-L-glutaminyl-L-tryptophyl-L-alanyl-L-valyl-glycyl-L-histidyl-L-leucyl-L-methioninamide (L70), as described in Example II.

FIG. 2B is a general graphical representation of the sequential reaction for the synthesis of N-[4-[2-[[[4,7,10-Tris(carboxymethyl)-1,4,7,10-tetraazacyclododec-1-yl]acetyl]amino]ethoxy]benzoyl]-L-glutaminyl-L-tryptophyl-L-alanyl-L-valyl-glycyl-L-histidyl-L-leucyl-L-methioninamide (L73), N-[3-[[[[4,7,10-Tris(carboxymethyl)-1,4,7,10-tetraazacyclododec-1-yl]acetyl]amino]methyl]benzoyl]-L-glutaminyl-L-tryptophyl-L-alanyl-L-valyl-glycyl-L-histidyl-L-leucyl-L-methioninamide (L115), and N-[4-[[[[4,7,10-Tris(carboxymethyl)-1,4,7,10-tetraazacyclododec-1-yl]acetyl]amino]methyl]phenylacetyl]-L-glutaminyl-L-tryptophyl-L-alanyl-L-valyl-glycyl-L-histidyl-L-leucyl-L-methioninamide (L116), as described in Example II.

FIG. 2C is a chemical structure of the linker used in the synthesis reaction of FIG. 2B for synthesis of N-[4-[2-[[[4,7,10-Tris(carboxymethyl)-1,4,7,10-tetraazacyclododec-1-yl]acetyl]amino]ethoxy]benzoyl]-L-glutaminyl-L-tryptophyl-L-alanyl-L-valyl-glycyl-L-histidyl-L-leucyl-L-methioninamide (L73), as described in Example II.

FIG. 2D is a chemical structure of the linker used in the synthesis reaction of FIG. 2B for synthesis of N-[3-[[[[4,7,10-Tris(carboxymethyl)-1,4,7,10-tetraazacyclododec-1-yl]acetyl]amino]methyl]benzoyl]-L-glutaminyl-L-tryptophyl-L-alanyl-L-valyl-glycyl-L-histidyl-L-leucyl-L-methioninamide (L115), as described in Example II.

FIG. 2E is a chemical structure of the linker used in the synthesis reaction of FIG. 2B for synthesis of N-[4-[[[[4,7,10-Tris(carboxymethyl)-1,4,7,10-tetraazacyclododec-1-yl]acetyl]amino]methyl]phenylacetyl]-L-glutaminyl-L-tryptophyl-L-alanyl-L-valyl-glycyl-L-histidyl-L-leucyl-L-methioninamide (L116), as described in Example II.

FIG. 2F is a graphical representation of the sequential reaction for the synthesis of N-[[4,7,10-Tris(carboxymethyl)-1,4,7,10-tetraazacyclododec-1-yl]acetyl]glycyl-4-piperidinecarbonyl-L-glutaminyl-L-tryptophyl-L-alanyl-L-valyl-glycyl-L-histidyl-L-leucyl-L-methioninamide (L74), as described in Example II.

FIG. 3A is a graphical representation of a series of chemical reactions for the synthesis of intermediate (3β,5β)-3-[[(9H-Fluoren-9-ylmethoxy)amino]acetyl]amino-12-oxocholan-24-oic acid (C), as described in Example III.

FIG. 3B is a graphical representation of the sequential reaction for the synthesis of N-[(3β,5β)-3-[[[[[4,7,10-Tris(carboxymethyl)-1,4,7,10-tetraazacyclododec-1-yl]acetyl]amino]acetyl]amino]-12,24-dioxocholan-24-yl]-L-glutaminyl-L-tryptophyl-L-alanyl-L-valyl-glycyl-L-histidyl-L-leucyl-L-methioninamide (L67), as described in Example III.

FIG. 3C is a chemical structure of (3β,5β)-3-Amino-12-oxocholan-24-oic acid (B), as described in Example III.

FIG. 3D is a chemical structure of (3β,5β)-3-[[(9H-Fluoren-9-ylmethoxy)amino]acetyl]amino-12-oxocholan-24-oic acid (C), as described in Example III.

FIG. 3E is a chemical structure of N-[(3β,5β)-3-[[[[[4,7,10-Tris(carboxymethyl)-1,4,7,10-tetraazacyclododec-1-yl]acetyl]amino]acetyl]amino]-12,24-dioxocholan-24-yl]-L-glutaminyl-L-tryptophyl-L-alanyl-L-valyl-glycyl-L-histidyl-L-leucyl-L-methioninamide (L67), as described in Example III.

FIG. 4A is a graphical representation of a sequence of reactions to obtain intermediates (3β,5β,12α)-3-[[(9H-Fluoren-9-ylmethoxy)amino]acetyl]amino-12-hydroxycholan-24-oic acid (3a) and (3β,5β,7α, 12α)-3-[[(9H-Fluoren-9-ylmethoxy)amino]acetyl]amino-7,12-dihydroxycholan-24-oic acid (3b), as described in Example IV.

FIG. 4B is a graphical representation of the sequential reaction for the synthesis of N-[(3β,5β,12α)-3-[[[[[4,7,10-Tris(carboxymethyl)-1,4,7,10-tetraazacyclododec-1-yl]acetyl]amino]acetyl]amino]-12-hydroxy-24-oxocholan-24-yl]-L-glutaminyl-L-tryptophyl-L-alanyl-L-valyl-glycyl-L-histidyl-L-leucyl-L-methioninamide (L63), as described in Example IV.

FIG. 4C is a graphical representation of the sequential reaction for the synthesis of N-[(3β,5β,7α,12α)-3-[[[[[4,7,10-Tris(carboxymethyl)-1,4,7,10-tetraazacyclo dodec-1-yl]acetyl]amino]acetyl]amino]-7,12-dihydroxy-24-oxocholan-24-yl]-L-glutaminyl-L-tryptophyl-L-alanyl-L-valyl-glycyl-L-histidyl-L-leucyl-L-methioninamide (L64), as described in Example IV.

FIG. 4D is a chemical structure of (3β,5β,7α,12α)-3-amino-7,12-dihydroxycholan-24-oic acid (2b), as described in Example IV.

FIG. 4E is a chemical structure of (3β,5β,12α)-3-[[(9H-Fluoren-9-ylmethoxy)amino]acetyl]amino-12-hydroxycholan-24-oic acid (3a), as described in Example IV;

FIG. 4F is a chemical structure of (3β,5β,7α,12α)-3-[[(9H-Fluoren-9-ylmethoxy)amino]acetyl]amino-7,12-dihydroxycholan-24-oic acid (3b), as described in Example IV.

FIG. 4G is a chemical structure of N-[(3β,5β,12α)-3-[[[[[4,7,10-Tris(carboxymethyl)-1,4,7,10-tetraazacyclododec-1-yl]acetyl]amino]acetyl]amino]-12-hydroxy-24-oxocholan-24-yl]-L-glutaminyl-L-tryptophyl-L-alanyl-L-valyl-glycyl-L-histidyl-L-leucyl-L-methioninamide (L63), as described in Example IV.

FIG. 4H is a chemical structure of N-[(3β,5β7α,12α)-3-[[[[[4,7,10-Tris(carboxymethyl)-1,4,7,10-tetraazacyclo dodec-1-yl]acetyl]amino]acetyl]amino]-7,12-dihydroxy-24-oxocholan-24-yl]-L-glutaminyl-L-tryptophyl-L-alanyl-L-valyl-glycyl-L-histidyl-L-leucyl-L-methioninamide (L64), as described in Example IV.

FIG. 5A is a general graphical representation of the sequential reaction for the synthesis of 4-[[[[4,7,10-Tris(carboxymethyl)-1,4,7,10-tetraazacyclododec-1-yl]acetyl]amino]methyl]benzoyl-L-glutaminyl-L-tryptophyl-L-alanyl-L-valyl-glycyl-L-histidyl-L-leucyl-L-methioninamide (L71); and Trans-4-[[[[4,7,10-tris(carboxymethyl)-1,4,7,10-tetraazacyclododec-1-yl]acetyl]amino]methyl]cyclohexylcarbonyl-L-glutaminyl-L-tryptophyl-L-alanyl-L-valyl-glycyl-L-histidyl-L-leucyl-L-methioninamide (L72) as described in Example V, wherein the linker is from FIG. 5B and FIG. 5C, respectively.

FIG. 5B is a chemical structure of the linker used in compound L71 as shown in FIG. 5A and as described in Example V.

FIG. 5C is a chemical structure of the linker used in compound L72 as shown in FIG. 5A and as described in Example V.

FIG. 5D is a chemical structure of Rink amide resin functionalised with bombesin[7-14](B), as described in Example V.

FIG. 5E is a chemical structure of Trans-4-[[[(9H-fluoren-9-ylmethoxy)carbonyl]amino]methyl]cyclohexanecarboxylic acid (D), as described in Example V;

FIG. 6A is a graphical representation of a sequence of reactions for the synthesis of intermediate linker 2-[[[9H-Fluoren-9-ylmethoxy)carbonyl]amino]methyl]benzoic acid (E), as described in Example VI.

FIG. 6B is a graphical representation of a sequence of reactions for the synthesis of intermediate linker 4-[[[9H-Fluoren-9-ylmethoxy)carbonyl]amino]methyl]-3-nitrobenzoic acid (H), as described in Example VI.

FIG. 6C is a graphical representation of the synthesis of N-[2-[[[[4,7,10-Tris(carboxymethyl)-1,4,7,10-tetraazacyclododec-1-yl]acetyl]amino]methyl]benzoyl]-L-glutaminyl-L-tryptophyl-L-alanyl-L-valyl-glycyl-L-histidyl-L-leucyl-L-methioninamide (L75), as described in Example VI.

FIG. 6D is a graphical representation of the synthesis of N-[4-[[[[4,7,10-Tris(carboxymethyl)-1,4,7,10-tetraazacyclododec-1-yl]acetyl]amino]methyl]-3-nitrobenzoyl]-L-glutaminyl-L-tryptophyl-L-alanyl-L-valyl-glycyl-L-histidyl-L-leucyl-L-methioninamide (L76), as described in Example VI.

FIG. 7A is a graphical representation of a sequence of reactions for the synthesis of intermediate linker [4-[[[9H-Fluoren-9-ylmethoxy)carbonyl]amino]methyl]phenoxy]acetic acid (E), as described in Example VII.

FIG. 7B is a graphical representation of the synthesis of N-[[4-[[[[4,7,10-Tris(carboxymethyl)-1,4,7,10-tetraazacyclododec-1-yl]acetyl]amino]methyl]phenoxy]acetyl]-L-glutaminyl-L-tryptophyl-L-alanyl-L-valyl-glycyl-L-histidyl-L-leucyl-L-methioninamide (L124), as described in Example VII.

FIG. 7C is a chemical structure of N-[[4-[[[[4,7,10-Tris(carboxymethyl)-1,4,7,10-tetraazacyclododec-1-yl]acetyl]amino]methyl]phenoxy]acetyl]-L-glutaminyl-L-tryptophyl-L-alanyl-L-valyl-glycyl-L-histidyl-L-leucyl-L-methioninamide (L124), as described in Example VII.

FIG. 8A is a graphical representation of a sequence of reactions for the synthesis of intermediate 4-[[[9H-Fluoren-9-ylmethoxy)carbonyl]amino]methyl]-3-methoxybenzoic acid (E), as described in Example VIII.

FIG. 8B is a graphical representation of the synthesis of N-[4-[[[[4,7,10-Tris(carboxymethyl)-1,4,7,10-tetraazacyclododec-1-yl]acetyl]amino]methyl]-3-methoxybenzoyl]-L-glutaminyl-L-tryptophyl-L-alanyl-L-valyl-glycyl-L-histidyl-L-leucyl-L-methioninamide, (L125), as described in Example VIII.

FIG. 8C is a chemical structure of N-[4-[[[[4,7,10-Tris(carboxymethyl)-1,4,7,10-tetraazacyclododec-1-yl]acetyl]amino]methyl]-3-methoxybenzoyl]-L-glutaminyl-L-tryptophyl-L-alanyl-L-valyl-glycyl-L-histidyl-L-leucyl-L-methioninamide, (L125), as described in Example VIII.

FIG. 9A is a graphical representation of a reaction for the synthesis of 3-[[[(9H-Fluoren-9-ylmethoxy)carbonyl]amino]acetyl]aminobenzoic acid, (B), as described in Example IX.

FIG. 9B is a graphical representation of a reaction for the synthesis of 6-[[[(9H-Fluoren-9-ylmethoxy)carbonyl]amino]acetyl]aminonaphthoic acid (C), as described in Example IX.

FIG. 9C is a graphical representation of a reaction for the synthesis of 4-[[[[(9H-Fluoren-9-ylmethoxy)carbonyl]amino]acetyl]methylamino]benzoic acid, (D), as described in Example IX.

FIG. 9D is a graphical representation of a reaction for the synthesis of N-[4-[[[[[4,7,10-Tris(carboxymethyl)-1,4,7,10-tetraazacyclododec-1-yl]acetyl]amino]acetyl]amino]phenylacetyl]-L-glutaminyl-L-tryptophyl-L-alanyl-L-valyl-glycyl-L-histidyl-L-leucyl-L-methioninamide, (L146); N-[3-[[[[[4,7,10-Tris(carboxymethyl)-1,4,7,10-tetraazacyclododec-1-yl]acetyl]amino]acetyl]amino]benzoyl]-L-glutaminyl-L-tryptophyl-L-alanyl-L-valyl-glycyl-L-histidyl-L-leucyl-L-methioninamide (L233); N-[6-[[[[[4,7,10-Tris(carboxymethyl)-1,4,7,10-tetraazacyclododec-1-yl]acetyl]amino]acetyl]amino]naphthoyl]-L-glutaminyl-L-tryptophyl-L-alanyl-L-valyl-glycyl-L-histidyl-L-leucyl-L-methioninamide, (L234), and N-[4-[[[[[4,7,10-Tris(carboxymethyl)-1,4,7,10-tetraazacyclododec-1-yl]acetyl]amino]acetyl]methylamino]benzoyl]-L-glutaminyl-L-tryptophyl-L-alanyl-L-valyl-glycyl-L-histidyl-L-leucyl-L-methioninamide, (L235), as described in Example IX.

FIG. 10A is a graphical representation of a reaction for the synthesis of 7-[[Bis(1,1-dimethylethoxy)phosphinyl]methyl]-1,4,7,10-tetraazacyclododecane-1,4,10-triacetic acid 4,10-bis(1,1-dimethylethyl) ester H, as described in Example X.

FIG. 10B is a graphical representation of a reaction for the synthesis of N-[4-[[[[[4,10-Bis(carboxymethyl)-7-(dihydroxyphosphinyl)methyl-1,4,7,100-tetraazacyclododec-1-yl]acetyl]amino]acetyl]amino]benzoyl]-L-glutaminyl-L-tryptophyl-L-alanyl-L-valyl-glycyl-L-histidyl-L-leucil-L-methioninamide, (L237), as described in Example X.

FIG. 11A is a graphical representation of a reaction for the synthesis of N,N-Dimethylglycyl-L-serinyl-[S-[(acetylamino)methyl]]-L-cysteinyl-glycyl-4-aminobenzoyl-L-glutaminyl-L-tryptophyl-L-alanyl-L-valyl-glycyl-L-histidyl-L-leucyl-L-methioninamide (L238), as described in Example XI.

FIG. 11B is a graphical representation of a reaction for the synthesis of N,N-Dimethylglycyl-L-serinyl-[S-[(acetylamino)methyl]]-L-cysteinyl-glycyl-(3β,5β,7α,12α)-3-amino-7,12-dihydroxy-24-oxocholan-24-yl-L-glutaminyl-L-tryptophyl-L-alanyl-L-valyl-glycyl-L-histidyl-L-leucyl-L-methioninamide, (L239), as described in Example XI.

FIG. 12A is a graphical representation of a reaction for the synthesis of 4-[[[(9H-Fluoren-9-ylmethoxy)carbonyl]amino]acetyl]amino-3-methoxybenzoic acid (A), as described in Example XII.

FIG. 12B is a graphical representation of a reaction for the synthesis of 4-[[[(9H-Fluoren-9-ylmethoxy)carbonyl]amino]acetyl]amino-3-chlorobenzoic acid, (D), as described in Example XII.

FIG. 12C is a graphical representation of a reaction for the synthesis of 4-[[[(9H-Fluoren-9-ylmethoxy)carbonyl]amino]acetyl]amino-3-methylbenzoic acid (E), as described in Example XII.

FIG. 12D is a chemical structure of N-[4-[[[[4,7,10-Tris(carboxymethyl)-1,4,7,10-tetraazacyclododec-1-yl]acetyl]glycyl]amino]-3-methoxybenzoyl]-L-glutaminyl-L-tryptophyl-1-alanyl-L-valyl-glycyl-L-histidyl-L-leucyl-L-methioninamide (L240) as described in Example XII.

FIG. 12E is a chemical structure of compound N-[4-[[[[4,7,10-Tris(carboxymethyl)-1,4,7,10tetraazacyclododec-1-yl]acetyl]glycyl]amino]3-chlorobenzoyl]L-glutaminyl-L-tryptophyl-1-alanyl-L-valyl-glycyl-L-histidyl-L-leucyl-L-methioninamide, (L241) as described in Example XII.

FIG. 12F is a chemical structure of N-[4-[[[[4,7,10-Tris(carboxymethyl)-1,4,7,10tetraazacyclododec-1-yl]acetyl]glycyl]amino]3-methylbenzoyl]L-glutaminyl-L-tryptophyl-1-alanyl-L-valyl-glycyl-L-histidyl-leucyl-L-methioninamide (L242), as described in Example XII.

FIG. 13A is a graphical representation of a reaction for the synthesis of 4-[N,N′-Bis[2-[(9-H-fluoren-9-ylmethoxy)carbonyl]aminoethyl]amino]-4-oxobutanoic acid, (D), as described in Example XIII.

FIG. 13B is a graphical representation of a reaction for the synthesis of N-[4-[[4-[Bis[2-[[[4,7,10-tris(carboxymethyl)-1,4,7,10-tetraazacyclododec-1-yl]acetyl]amino]ethyl]amino-1,4-dioxobutyl]amino]benzoyl]-L-glutaminyl-L-tryptophyl-L-alanyl-L-valyl-glycyl-L-histidyl-L-leucyl-L-methioninamide, (L244), as described in Example XIII.

FIG. 13C is a chemical structure of compound L244, as described in Example XIII.

FIG. 14A and FIG. 14B are graphical representations of the binding and competition curves described in Example XLIII.

FIG. 15A is a graphical representation of the results of radiotherapy experiments described in Example LV.

FIG. 15B is a graphical representation of the results of other radiotherapy experiments described in Example LV.

FIG. 16 is a chemical structure of N-[4-[[[[4,7,10-Tris(carboxymethyl)-1,4,7,10 tetraazacyclododec-11-yl]acetyl]glycyl]amino]-L-Lysinyl-(3,6,9)-trioxaundecane-1,11-dicarboxylic acid-3,7-dideoxy-3-aminocholic acid)-L-arginyl-L-glutaminyl-L-triptophyl-L-alanyl-L-valyl-glycyl-L-histidyl-L-leucyl-L-methioninamide (L65).

FIG. 17 is a chemical structure of N-[2-S-[[[[[12α-Hydroxy-17α-(1-methyl-3-carboxypropyl)etiocholan-33-carbamoylmethoxyethoxyethoxyacetyl]-amino-6-[4,7,10-tris(carboxymethyl)-1,4,7,10-tetraazacyclododec-1-yl]acetyl]amino]acetyl]amino]hexanoyl-L-glutaminyl-L-tryptophyl-L-alanyl-L-valyl-glycyl-L-histidyl-L-leucyl-L-methioninamide (L66).

FIG. 18A is a chemical structure of N-[4-[[[[[4,7,10-Tris(carboxymethyl)-1,4,7,10-tetraazacyclododec-1-yl]acetyl]amino]acetyl]amino]benzoyl]-L-glutaminyl-L-tryptophyl-L-alanyl-L-valyl-glycyl-L-histidyl-L-leucyl-L-methioninamide (L70).

FIG. 18B is a chemical structure N-[4-[[[[[4,7,10-Tris(carboxymethyl)-1,4,7,10-tetraazacyclododec-1-yl]-3-carboxypropionyl]amino]acetyl]amino]benzoyl]-L-glutaminyl-L-tryptophyl-L-alanyl-L-valyl-glycyl-L-histidyl-L-leucyl-L-methioninamide (L114).

FIG. 18C is a chemical structure N-[4-[[4,7,10-Tris(carboxymethyl)-1,4,7,10-tetraazacyclododec-1-yl]-2-hydroxy-3-propoxy]benzoyl]-L-glutaminyl-L-tryptophyl-L-alanyl-L-valyl-glycyl-L-histidyl-L-leucyl-L-methioninamide (L144).

FIG. 18D is a chemical structure N-[(3β,5β,7α,12α)-3-[[[[[[4,7,10-Tris(carboxymethyl)-1,4,7,10-tetraazacyclododec-1-yl]acetyl]amino]ethoxyethoxy]acetyl]amino]-7,12-dihydroxycholan-24-yl]-L-glutaminyl-L-tryptophyl-L-alanyl-L-valyl-glycyl-L-histidyl-L-leucyl-L-methioninamide (L69).

FIG. 18E is a chemical structure of N-[4-[[[[[4,7,10-Tris(carboxymethyl)-1,4,7,10-tetraazacyclododec-1-yl]acetyl]amino]acetyl]amino]phenylacetyl]-L-glutaminyl-L-tryptophyl-L-alanyl-L-valyl-glycyl-L-histidyl-L-leucyl-L-methioninamide (L146).

FIG. 19 discloses chemical structures of intermediates which may be used to prepare compounds L64 and L70 as described in Example LVI.

FIG. 20 is a graphical representation of the preparation of L64 using segment coupling as described in Example LVI.

FIG. 21 is a graphical representation of the preparation of (1R)-1-(Bis{2-[bis(carboxymethyl)amino]ethyl}amino)propane-3-carboxylic acid-1-carboxyl-glycyl-4-aminobenzoyl-L-glutaminyl-L-tryptophyl-L-alanyl-L-valyl-glycyl-L-histidyl-L-leucyl-L-methioninamide (L201).

FIG. 22A is a graphical representation of chemical structure of chemical intermediates used to prepare L202.

FIG. 22B is a graphical representation of the preparation of N-[(3β,5β,12α)-3-[[[[[4,7,10-Tris(carboxymethyl)-1,4,7,10-tetraazacyclododec-1-yl]acetyl]amino]acetyl]amino]-4-hydrazinobenzoyl-L-glutaminyl-L-tryptophyl-L-alanyl-L-valyl-glycyl-L-histidyl-L-leucyl-L-methioninamide (L202).

FIG. 23A is a graphical representation of chemical structure of chemical intermediates used to prepare L203.

FIG. 23B is a graphical representation of the preparation of N-[(3β,5β,12α)-3-[[[4,7,10-Tris(carboxymethyl)-1,4,7,10-tetraazacyclododec-1-yl]acetyl]amino]-4-aminobenzoyl-L-glutaminyl-L-tryptophyl-L-alanyl-L-valyl-glycyl-L-histidyl-L-leucyl-L-methioninamide (L203).

FIG. 24 is a graphical representation of the preparation of N-[(3β,5β,12α)-3-[[[4,7,10-Tris(carboxymethyl)-1,4,7,10-tetraazacyclododec-1-yl]acetyl]amino]-4-aminobenzoyl-glycyl-L-glutaminyl-L-tryptophyl-L-alanyl-L-valyl-glycyl-L-histidyl-L-leucyl-L-methioninamide (L204).

FIG. 25 is a graphical representation of the preparation of N-[(3β,5β,12α)-3-[[[4,7,10-Tris(carboxymethyl)-1,4,7,10-tetraazacyclododec-1-yl]acetyl]amino]-4-aminobenzoyl-glycyl-L-glutaminyl-L-tryptophyl-L-alanyl-L-valyl-glycyl-L-histidyl-L-leucyl-L-methioninamide (L205).

FIG. 26A is a graphical representation of chemical structures of chemical intermediates used to prepare L206.

FIG. 26B is a graphical representation of the preparation of N-[(3β,5β,12α)-3-[[[[[4,7,10-Tris(carboxymethyl)-1,4,7,10-tetraazacyclododec-1-yl]acetyl]amino]acetyl]amino]-[4′-Amino-2′-methyl biphenyl-4-carboxyl]-L-glutaminyl-L-tryptophyl-L-alanyl-L-valyl-glycyl-L-histidyl-L-leucyl-L-methioninamide (L206).

FIG. 27A is a graphical representation of chemical structures of chemical intermediates used to prepare L207.

FIG. 27B is a graphical representation of the preparation of N-[(3β,5β,12α)-3-[[[[[4,7,10-Tris(carboxymethyl)-1,4,7,10-tetraazacyclododec-1-yl]acetyl]amino]acetyl]amino]-[3′-amino-biphenyl-3-carboxyl]-L-glutaminyl-L-tryptophyl-L-alanyl-L-valyl-glycyl-L-histidyl-L-leucyl-L-methioninamide (L207).

FIG. 28 is a graphical representation of the preparation of N-[(3β,5β,12α)-3-[[[[[4,7,10-Tris(carboxymethyl)-1,4,7,10-tetraazacyclododec-1-yl]acetyl]amino]acetyl]amino]-[1,2-d]aminoethyl-terephthalyl]-L-glutaminyl-L-tryptophyl-L-alanyl-L-valyl-glycyl-L-histidyl-L-leucyl-L-methioninamide (L208).

FIG. 29A is a graphical representation of chemical structures of chemical intermediates used to prepare L209.

FIG. 29B is a graphical representation of the preparation of L209.

FIG. 30A is a graphical representation of chemical structures of chemical intermediates used to prepare L210.

FIG. 30B is a chemical structure of L210.

FIG. 31 is a chemical structure of N-[(3β,5β,12α)-3-[[[4,7,10-Tris(carboxymethyl)-1,4,7,10-tetraazacyclododec-1-yl]acetyl]amino]-glycyl-glycyl-4-aminobenzoyl-L-glutaminyl-L-tryptophyl-L-alanyl-L-valyl-glycyl-L-histidyl-L-leucyl-L-methioninamide L211.

FIG. 32 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutamyl-L-tryptophyl-L-alanyl-L-valyl-glycyl-L-histidyl-L-leucyl-L-methioninamide L212.

FIG. 33 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutaminyl-L-tryptophyl-L-alanyl-L-valyl-glycyl-L-histidyl-L-leucyl-L-methionine carboxylate L213.

FIG. 34 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-D-phenylalanyl-L-glutaminyl-L-tryptophyl-L-alanyl-L-valyl-glycyl-L-histidyl-L-leucyl-L-methioninamide L214.

FIG. 35 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutaminyl-L-arginyl-L-leucyl-glycyl-L-asparginyl-L-glutaminyl-L-tryptophyl-L-alanyl-L-valyl-glycyl-L-histidyl-L-leucyl-L-methioninamide L215.

FIG. 36 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutaminyl-arginyl-L-tyrosinyl-glycyl-L-asparginyl-L-glutaminyl-L-tryptophyl-L-alanyl-L-valyl-glycyl-L-histidyl-L-leucyl-L-methioninamide L216.

FIG. 37 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutaminyl-L-lysyl-L-tyrosinyl-glycyl-L-glutaminyl-L-tryptophyl-L-alanyl-L-valyl-glycyl-L-histidyl-L-leucyl-L-methioninamide L217.

FIG. 38 is a chemical structure of L218.

FIG. 39 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-D-phenylalanyl-L-glutaminyl-L-tryptophyl-L-alanyl-L-valyl-glycyl-L-histidyl-L-leucyl-aminopentyl, L219.

FIG. 40 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutaminyl-L-tryptophyl-L-serinyl-L-valyl-D-alanyl-L-histidyl-L-leucyl-L-methioninamide, L220.

FIG. 41 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-D-phenylalanyl-L-glutaminyl-L-tryptophyl-L-alanyl-L-valyl-glycyl-L-histidyl-L-leucyl-L-leucinamide, L221.

FIG. 42 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-D-tyrosinyl-L-glutaminyl-L-tryptophyl-L-alanyl-L-valyl-betaalanyl-L-histidyl-L-phenylalanyl-L-norleucinamide, L222.

FIG. 43 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-L-phenylalanyl-L-glutaminyl-L-tryptophyl-L-alanyl-L-valyl-betaalanyl-L-histidyl-L-phenylalanyl-L-norleucinamide, L223.

FIG. 44 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutaminyl-L-tryptophyl-L-alanyl-glycyl-L-histidyl-L-phenylalanyl-L-leucinamide, L224.

FIG. 45 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-L-leucyl-L-tryptophyl-L-alanyl-L-valinyl-glycyl-L-serinyl-L-phenylalanyl-L-methioninamide, L225.

FIG. 46 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-L-histidyl-L-tryptophyl-L-alanyl-L-valyl-glycyl-L-histidyl-L-leucyl-L-methioninamide, L226.

FIG. 47 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-L-leucyl-L-tryptophyl-L-alanyl-L-valyl-glycyl-L-serinyl-L-phenylalanyl-L-methioninamide L227.

FIG. 48 is a chemical structure of N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutaminyl-L-tryptophyl-L-alanyl-L-valyl-glycyl-L-histidyl-L-phenylalanyl-L-methioninamide, L228.

FIG. 49A is a graphical representation of a reaction for the synthesis of (3β,5β,7α,12α)-3-(9H-Fluoren-9-ylmethoxy)amino-7,12-dihydroxycholan-24-oic acid (B) as described in Example LVII.

FIG. 49B is a graphical representation of a reaction for the synthesis of N-[3β,5β,7α,12α)-3-[[[2-[2-[[[4,7,10-Tris(carboxymethyl)-1,4,7,10-tetraazacyclododec-1-yl]acetyl]amino]ethoxy]ethoxy]acetyl]amino]-7,12-dihydroxy-24-oxocholan-24-yl]-L-glutaminyl-L-tryptophyl-L-alanyl-L-valyl-glycyl-L-histidyl-L-leucyl-L-methioninamide, (L69), as described in Example LVII.

FIG. 50 is a graphical representation of a reaction for the synthesis of N-[4-[2-Hydroxy-3-[4,7,10-tris(carboxymethyl)-1,4,7,10-tetraazacyclododec-1-yl]propoxy]benzoyl]-L-glutaminyl-L-tryptophyl-L-alanyl-L-valyl-glycyl-L-histidyl-L-leucyl-L-methioninamide (L144), as described in Example LVIII.

FIG. 51 is a chemical structure of L300.

FIG. 52 is a chemical structure of L301.

FIG. 53 is a graphical representation of the preparation of L500 as described in Example LXII.

FIG. 54 is a graphical representation of the preparation of L501 as described in Example LXIII.

FIG. 55 is a graphical representation of occurrence of ecchymosis in control and ¹⁷⁷Lu-AMBA (¹⁷⁷Lu-L70) treated mice over time.

FIG. 56 is a photograph of a control group mouse from an experiment determining the occurrence of ecchymosis in control and ¹⁷⁷Lu-AMBA (¹⁷⁷Lu-L70) treated mice over time.

FIG. 57 is a photograph of an experimental group mouse from an experiment determining the occurrence of ecchymosis in control and ¹⁷⁷Lu-AMBA (¹⁷⁷Lu-L70) treated mice over time.

FIG. 58 is a diagram of crosstalk pathways for the GRP Receptor.

FIG. 59 shows targeting and imaging of cancers by ⁶⁸Ga-AMBA.

### ABBREVIATIONS USED IN THE APPLICATION

| | |
|---|---|
| Aazta | 1,4-Bis(carboxymethyl)-6-[bis(carboxymethyl)amino]- |
| | 6-methyl-perhydro-1,4-diazepine |
| CyAazta | 2-{[(1S,7S)-2,6-diaza-2,6-bis(carboxymethyl)-4- |
| | methylbicyclo[5.4.0]undec-4- |
| | yl]carboxymethyl)amino}acetic acid |
| Aoc- | 8-aminooctanoic acid |
| Apa3- | 3-aminopropionic acid |
| Abu4- | 4-aminobutanoic acid |
| Adca3- | (3β,5β 7α,12α)-3-amino-7,12-dihydroxycholan-24-oic |
| | acid or 3-Amino-3-deoxycholic acid |
| Ah12ca- | (3β,5β,12α)-3-amino-12-hydroxycholan-24-oic acid |
| Akca- | (3β,5β,7α,12α)-3-amino-12-oxacholan-24-oic acid |
| Cha- | L-Cyclohexylalanine |
| Nal1- | L-1-Naphthylalanine |
| Bip- | L-Biphenylalanine |
| Mo3abz4- | 3-Methoxy-4-aminobenzoic acid or 4-aminomethyl-3- |
| | methoxybenzoic acid |
| Bpa4- | 4-benzoylphenylalanine |
| Cl3abz4- | 3-Chloro-4-aminobenzoic acid |
| M3abz4- | 3-methyl-4-aminobenzoic acid |
| Ho3abz4- | 3-hydroxy-4-aminobenzoic acid |
| Hybz4- | 4-hydrazinobenzoic acid |
| Nmabz4- | 4-methylaminobenzoic acid |
| Mo3amb4- | 3-methoxy-4-aminobenzoic acid |
| Amb4- | 4-aminomethylbenzoic acid |
| Aeb4- | 4-(2-aminoethoxy)benzoic acid |
| Dae- | 1,2-diaminoethyl |
| Tpa- | Terephthalic acid |
| A4m2biphc4- | 4′-Amino-2′-methyl biphenyl-4-carboxylic acid |
| A3biphc3- | 3-amino-3′-biphenylcarboxylic acid |
| Amc4- | trans-4-aminomethylcyclohexane carboxylic acid |
| Aepa4- | N-4-aminoethyl-N-1-piperazine-acetic acid |
| Inp- | Isonipecotic acid |
| Pia1- | N-1-piperazineacetic acid |
| Ckbp- | 4-(3-Carboxymethyl-2-keto-1-benzimidazoyl)- |
| | piperidine |
| Abz3 | 3-Aminobenzoic acid |
| Abz4 | 4-Aminobenzoic acid |
| J | 8-amino-3,6-dioxaoctanoic acid |
| Ava5 | 5-Aminovaleric acid |
| f | (D)-Phe |
| y | (D)-Tyr |
| Ala2 (also Bala) | Beta-alanine |

The following is a list of the chemotherapeutics used in the examples of the invention, the target(s) involved in crosstalk, the types of cancer they have approval for, and a partial list of applicable clinical trials:
tamoxifen (4OH-TAM): inhibits estrogen binding (estrogen receptor modulators—SERMs); approved for the treatment of breast cancer; in clinical trials for prostate, ovarian, bone, liver cancer and advanced solid tumors.
gefitinib: Kinase Inhibitor: EGFR; inhibits intracellular phosphorylation of tyrosine kinases; approved for non-small cell lung cancer (NSCLC); in clinical trials for breast, prostate, ovarian, esophageal, brain, liver, and kidney cancer.
dasatinib: Src Family Kinase Inhibitor; approved for CML; in clinical trials for breast, prostate brain, liver, lung, and colorectal cancer.
lapatinib: Dual Kinase Inhibitor: EGFR and HER2/ErbB2 (and ErbB3); approved for breast cancer; in clinical trials for prostate, ovarian, colorectal, brain cancer, and advanced solid tumors.
imatinib: Multiple Kinase Inhibitor: Bcr-Abl tyrosine kinase; also inhibits PDGF and stem cell factor (SCF), Kit, and inhibits PDGF- and SCF-mediated cellular events; approved for CML and gastrointestinal stromal tumor (GIST); in clinical trials for breast, ovarian, and prostate cancer.
erlotinib: Kinase Inhibitor: EGFR; approved for NSCLC and pancreatic cancer; in clinical trials for HER2 negative breast cancer, prostate, esophageal, colorectal and brain cancer.
sorafenib: Multiple Kinase Inhibitor: PDGFRba/VEGFR 1,2,3/KIT, FLT3/EGF/Ras/Raf kinase; approved for hepatocellular carcinoma (HCC) and renal cell carcinoma (RCC); in clinical trials for breast, prostate, brain, and advanced solid tumors.
sunitinib: Multiple Kinase Inhibitor: EGFR, HER2, ErbB3; PDGF and 3; stem cell factor receptor (KIT); FLT3; CSF-1R; neurotropic factor receptor (RET); approved for RCC and GIST; in clinical trials for breast, prostate, brain, colorectal, and advanced solid tumors.
anastrozole: aromatase inhibitor, blocks production of estrogen/estradiol (e.g. tumor generated estrogen); approved for breast cancer; in clinical trials for ovarian and advanced breast cancer.
bortezomib: Proteasome inhibitor; blocks ubiquitin pathway, leading to apoptosis; approved for multiple myeloma; in clinical trials for breast, prostate, cervical, ovarian, brain, colorectal, NSCLC, and advanced solid tumors.

### DETAILED DESCRIPTION OF THE INVENTION

In the following description, various aspects of the present invention will be further elaborated. For purposes of explanation, specific configurations and details are set forth in order to provide a thorough understanding of the present invention. However, it will also be apparent to one skilled in the art that the present invention may be practiced without the specific details. Furthermore, well known features may be omitted or simplified in order not to obscure the present invention.

In an embodiment of the present invention, there are provided new and improved compounds for use in diagnostic imaging or radiotherapy. The compounds include an optical label or a chemical moiety capable of complexing a medically useful metal ion or radionuclide (metal chelator) attached to a GRP receptor targeting peptide by a linker or spacer group.

In general, compounds of the present invention may have the formula:

M-N—O—P-G

wherein M is the metal chelator (in the form complexed with a metal radionuclide or not), or an optical label or a moiety that contains a radiolabeled halogen such as ¹⁸F, ¹²³I—, ¹²⁴I— or ¹³¹I—. N—O—P is the linker, and G is the GRP receptor targeting peptide. Each of the metal chelator, optical label, linker, and GRP receptor targeting peptide is described in the discussion that follow.

In the most preferred embodiment of the invention, M is a metal chelator and the linker N—O—P contains at least one non-alpha amino acid with a cyclic group. In another preferred embodiment M is a metal chelator of formula 8 herein (preferably an Aazta chelator or a derivative thereof) and the linker N—O—P contains at least one non-alpha amino acid with a cyclic group. In a more preferred embodiment M-N—O—P-Q is L70 or AMBA as described in herein. In an especially preferred embodiment L70 or AMBA is complexed ¹⁷Lu (¹⁷Lu-AMBA or ¹⁷Lu-L70) or with a radionuclide detectable by positron emission tomography (PET), such as ⁶⁸Ga (⁶⁸Ga-AMBA or ⁶⁸Ga-L70).

In another embodiment of the present invention, there is provided a new and improved linker or spacer group which is capable of linking an optical label or a metal chelator to a GRP receptor targeting peptide. In general, linkers of the present invention may have the formula:

N—O—P

wherein each of N, O and P are defined throughout the specification.

Compounds meeting the criteria defined herein were discovered to have improved pharmacokinetic properties compared to other GRP receptor targeting peptide conjugates known in the art. For example, compounds containing the linkers of the present invention were retained in the bloodstream longer, and thus had a longer half life than prior known compounds. The longer half life was medically beneficial because it permitted better tumor targeting which is useful for diagnostic imaging, and especially for therapeutic uses, where the cancerous cells and tumors receive greater amounts of the radiolabeled peptides. Additionally, compounds of the present invention had improved tissue receptor specificity compared to prior art compounds.

Furthermore, the instant invention includes a method of increasing targeting of a labeled compound of the invention to GRP receptor expressing target tissue comprising administering the appropriate mass of GRP receptor targeting peptide or conjugate, prior to or during administration of labeled compound of the invention. Similarly, the invention includes an improved method of administration of labeled compounds of the invention in which tumor targeting is optimized, comprising administering the appropriate mass dose of GRP receptor targeting peptide or conjugate prior to or during administration of labeled compound of the invention. Such pre- or co-dosing has been found to saturate non-target GRP-receptors, decreasing their ability to compete with GRP receptors on target (e.g., tumor) tissue.

The present invention also provides a method of examining crosstalk with GRP receptors, specifically cross talk in the RTK or “other target” to GRPR direction, for a broad spectrum of solid human tumors that express GRPR (primaries and metastases) including, but not limited to breast and prostate cancer. Specifically, the invention permits assessing the effect of treatment with any one of a broad class of therapeutics targeted to RTK receptors or the estrogen receptor (e.g. RTK inhibitors, estrogen inhibitors), administered under normal clinical conditions (dose and schedule), may have on the function of such receptors (e.g. RTK receptors or the estrogen receptor) as detected by changes in the expression of the GRP receptor specific signal with which they exhibit crosstalk. The invention provides a functional indication of the anticipated response to therapy via an increase, decrease, or no change in the GRP receptor specific signal activity in vivo. The invention also provides a method of screening new drugs for changes in the activity of the GRP receptor family in vitro using a radiolabeled or otherwise detectable labeled agonist or antagonist of the GRP receptors. The invention also provides a method of imaging the activity of the GRP receptor family in vivo using a radiolabeled agonist or antagonist of the GRP receptors. In particular the invention envisages the use of a radiolabeled bombesin analogue as described in U.S. Pat. No. 7,226,577, incorporated here by reference, to obtain images in vivo (PET and/or SPECT) to improve patient management.

### 1A. Metal Chelator

The term “metal chelator” refers to a molecule that forms a complex with a metal atom, wherein said complex is stable under physiological conditions. That is, the metal will remain complexed to the chelator backbone in vivo. More particularly, a metal chelator is a molecule that complexes to a radionuclide metal to form a metal complex that is stable under physiological conditions and which also has at least one reactive functional group for conjugation with the linker N—O—P. The metal chelator M may be any of the metal chelators known in the art for complexing a medically useful metal ion or radionuclide. The metal chelator may or may not be complexed with a metal radionuclide. Furthermore, the metal chelator can include an optional spacer such as, for example, a single amino acid (e.g., Gly) which does not complex with the metal, but which creates a physical separation between the metal chelator and the linker.

The metal chelators of the invention may include, for example, linear, macrocyclic, terpyridine, and N₃S, N₂S₂, or N₄ chelators (see also, U.S. Pat. No. 5,367,080, U.S. Pat. No. 5,364,613, U.S. Pat. No. 5,021,556, U.S. Pat. No. 5,075,099, U.S. Pat. No. 5,886,142, the disclosures of which are incorporated by reference in their entirety), and other chelators known in the art including, but not limited to, HYNIC, DTPA, EDTA, DOTA, TETA, and bisamino bisthiol (BAT) chelators (see also U.S. Pat. No. 5,720,934). For example, N₄ chelators are described in U.S. Pat. Nos. 6,143,274; 6,093,382; 5,608,110; 5,665,329; 5,656,254; and 5,688,487, the disclosures of which are incorporated by reference in their entirety. Certain N₃S chelators are described in PCT/CA94/00395, PCT/CA94/00479, PCT/CA95/00249 and in U.S. Pat. Nos. 5,662,885; 5,976,495; and 5,780,006, the disclosures of which are incorporated by reference in their entirety. The chelator may also include derivatives of the chelating ligand mercapto-acetyl-glycyl-glycyl-glycine (MAG3), which contains an N₃S, and N₂S₂ systems such as MAMA (monoamidemonoaminedithiols), DADS (N₂S diaminedithiols), CODADS and the like. These ligand systems and a variety of others are described in Liu and Edwards, Chem. Rev. 1999, 99, 2235-2268 and references therein, the disclosures of which are incorporated by reference in their entirety.

The metal chelator may also include complexes containing ligand atoms that are not donated to the metal in a tetradentate array. These include the boronic acid adducts of technetium and rhenium dioximes, such as those described in U.S. Pat. Nos. 5,183,653; 5,387,409; and 5,118,797, the disclosures of which are incorporated by reference in their entirety.

Examples of preferred chelators include, but are not limited to, diethylenetriamine pentaacetic acid (DTPA), 1,4,7,10-tetraazacyclotetradecane-1,4,7,10-tetraacetic acid (DOTA), 1-substituted 1,4,7,-tricarboxymethyl 1,4,7,10-tetraazacyclododecane triacetic acid (DO3A), ethylenediaminetetraacetic acid (EDTA), 4-carbonylmethyl-10-phosphonomethyl-1,4,7,10-Tetraazacyclododecane-1,7-diacetic acid (Cm4 μm10d2a); and 1,4,8,11-tetraazacyclotetradecane-1,4,8,11-tetraacetic acid (TETA). Additional chelating ligands are ethylenebis-(2-hydroxy-phenylglycine) (EHPG), and derivatives thereof, including 5-Cl-EHPG, 5-Br-EHPG, 5-Me-EHPG, 5-t-Bu-EHPG, and 5-sec-Bu-EHPG; benzodiethylenetriamine pentaacetic acid (benzo-DTPA) and derivatives thereof, including dibenzo-DTPA, phenyl-DTPA, diphenyl-DTPA, benzyl-DTPA, and dibenzyl-DTPA; bis-2 (hydroxybenzyl)-ethylene-diaminediacetic acid (HBED) and derivatives thereof; the class of macrocyclic compounds which contain at least 3 carbon atoms, more preferably at least 6, and at least two heteroatoms (O and/or N), which macrocyclic compounds can consist of one ring, or two or three rings joined together at the hetero ring elements, e.g., benzo-DOTA, dibenzo-DOTA, and benzo-NOTA, where NOTA is 1,4,7-triazacyclononane N,N′,N″-triacetic acid, benzo-TETA, benzo-DOTMA, where DOTMA is 1,4,7,10-tetraazacyclotetradecane-1,4,7,10-tetra(methyl tetraacetic acid), and benzo-TETMA, where TETMA is 1,4,8,11-tetraazacyclotetradecane-1,4,8,11-(methyl tetraacetic acid); derivatives of 1,3-propylenediaminetetraacetic acid (PDTA) and triethylenetetraaminehexaacetic acid (TTHA); derivatives of 1,5,10-N,N′,N″-tris(2,3-dihydroxybenzoyl)-tricatecholate (LICAM) and 1,3,5-N,N′,N″-tris(2,3-dihydroxybenzoyl)aminomethylbenzene (MECAM). Other preferred chelators include Aazta and derivatives thereof including CyAazta. Examples of representative chelators and chelating groups contemplated by the present invention are described in WO 98/18496, WO 86/06605, WO 91/03200, WO 95/28179, WO 96/23526, WO 97/36619, PCT/US98/01473, PCT/US98/20182, and U.S. Pat. No. 4,899,755, U.S. Pat. No. 5,474,756, U.S. Pat. No. 5,846,519 and U.S. Pat. No. 6,143,274, each of which is hereby incorporated by reference in its entirety.

Particularly preferred metal chelators include those of Formula 1, 2, 3 and 8 (for ¹¹¹In and radioactive lanthanides, such as, for example ¹⁷⁷Lu, ⁹⁰Y, ¹⁵³Sm, ⁶⁸Ga, and ¹⁶⁶Ho) and those of Formula 4, 5 and 6 (for radioactive ^{99m}Tc, ¹⁸⁶Re, and ¹⁸⁸Re) set forth below. These and other metal chelating groups are described in U.S. Pat. Nos. 6,093,382 and 5,608,110, which are incorporated by reference in their entirety. Additionally, the chelating group of formula 3 is described in, for example, U.S. Pat. No. 6,143,274; the chelating group of formula 5 is described in, for example, U.S. Pat. Nos. 5,627,286 and 6,093,382, and the chelating group of formula 6 is described in, for example, U.S. Pat. Nos. 5,662,885; 5,780,006; and 5,976,495, all of which are incorporated by reference. The chelating group of formula 8 is described in copending U.S. Ser. No. 10/484,111 filed Jan. 15, 2004 and U.S. Ser. No. 11/165,793, filed Jun. 23, 2005, both of which are hereby incorporated by reference. Specific metal chelators of formula 6 include N,N-dimethylGly-Ser-Cys; N,N-dimethylGly-Thr-Cys; N,N-diethylGly-Ser-Cys; N,N-dibenzylGly-Ser-Cys; and other variations thereof. For example, spacers which do not actually complex with the metal radionuclide such as an extra single amino acid Gly, may be attached to these metal chelators (e.g., N,N-dimethylGly-Ser-Cys-Gly; N,N-dimethylGly-Thr-Cys-Gly; N,N-diethylGly-Ser-Cys-Gly; N,N-dibenzylGly-Ser-Cys-Gly). Other useful metal chelators such as all of those disclosed in U.S. Pat. No. 6,334,996, also incorporated by reference (e.g., Dimethylgly-L-t-Butylgly-L-Cys-Gly; Dimethylgly-D-t-Butylgly-L-Cys-Gly; Dimethylgly-L-t-Butylgly-L-Cys, etc.)

Furthermore, sulfur protecting groups such as Acm (acetamidomethyl), trityl or other known alkyl, aryl, acyl, alkanoyl, aryloyl, mercaptoacyl and organothiol groups may be attached to the cysteine amino acid of these metal chelators.

Additionally, other useful metal chelators include:

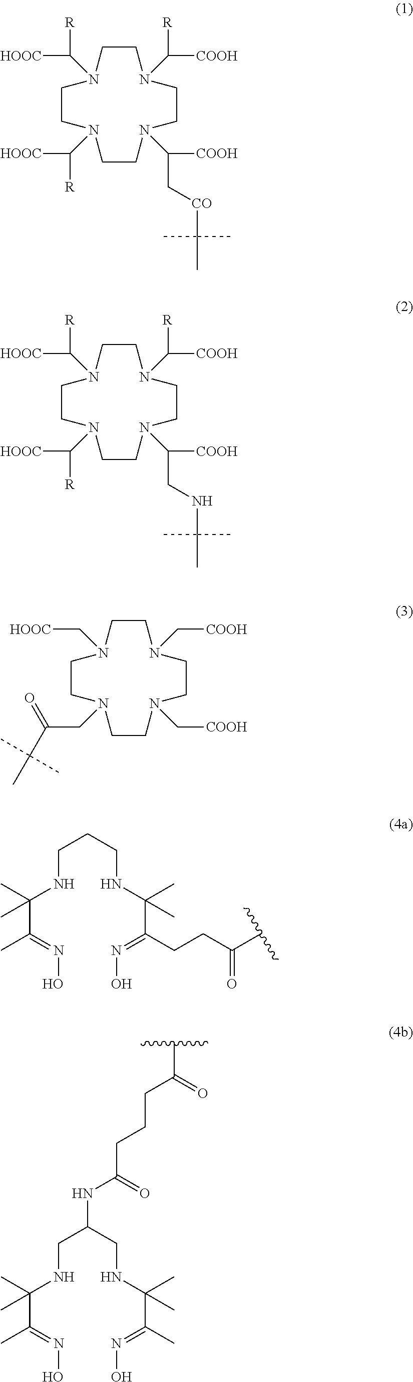

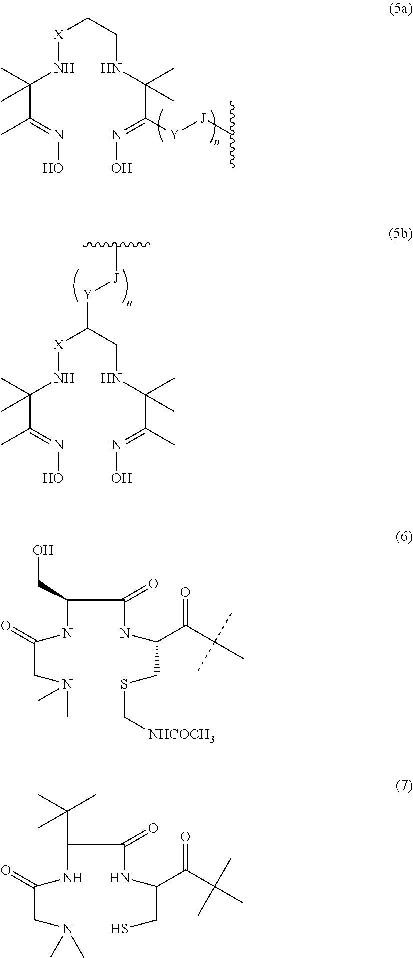

In the above Formulas 1 and 2, R is alkyl, preferably methyl. In the above Formulas 5a and 5b, X is either CH₂ or O; Y is C₁-C₁₀ branched or unbranched alkyl; aryl, aryloxy, arylamino, arylaminoacyl; arylalkyl—where the alkyl group or groups attached to the aryl group are C₁-C₁₀ branched or unbranched alkyl groups, C₁-C₁₀ branched or unbranched hydroxy or polyhydroxyalkyl groups or polyalkoxyalkyl or polyhydroxy-polyalkoxyalkyl groups; J is optional, but if present is C(═O)—, OC(═O)—, SO₂—, NC(═O)—, NC(═S)—, N(Y), NC(═NCH₃)—, NC(═NH)—, N═N—, homopolyamides or heteropolyamines derived from synthetic or naturally occurring amino acids; all where n is 1-100. Other variants of these structures are described, for example, in U.S. Pat. No. 6,093,382. In Formula 6, the group S—NHCOCH₃ may be replaced with SH or S—Z wherein Z is any of the known sulfur protecting groups such as those described above. Formula 7 illustrates one embodiment of t-butyl compounds useful as a metal chelator. The disclosures of each of the foregoing patents, applications and references are incorporated by reference in their entirety.

The metal chelators of the Aazta family generally have the following general formula:(8):

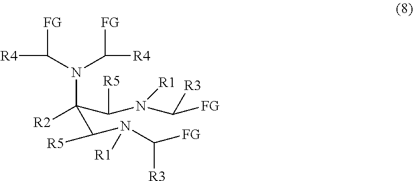

in which:

R₁ is hydrogen, C₁-C₂₀ alkyl optionally substituted with one or more carboxy groups, C₃-C₁₀ cycloalkyl, C₄-C₂₀ cycloalkylalkyl, aryl, arylalkyl or the two R₁ groups, taken together, form a straight or cyclic C₂-C₁₀ alkylene group or an ortho-disubstituted arylene;

R₂ is hydrogen, carboxy, or an optionally substituted group selected from C₁-C₂₀ alkyl, C₃-C₁₀ cycloalkyl, C₄-C₂₀ cycloalkylalkyl, aryl, arylalkyl, a group bearing an acidic moiety, and a group bearing an amino moiety, each of which may be further optionally substituted with functional groups which allow conjugation with a suitable molecule able to interact with physiological systems;

R₃, R₄ and R₅, which can be the same or different, are hydrogen, carboxy, or an optionally substituted group selected from C₁-C₂₀ alkyl, C₃-C₁₀ cycloalkyl, C₄-C₂₀ cycloalkylalkyl, aryl, arylalkyl, a group bearing an acidic moiety and a group bearing an amino moiety, each of which may be further optionally substituted with functional groups which allow conjugation with a suitable molecule able to interact with physiological systems; FG, which can be the same or different, are carboxy, —PO₃H₂ or —RP(O)OH groups, wherein R is hydrogen, or an optionally substituted group selected from C₁-C₂₀ alkyl, C₃-C₁₀ cycloalkyl, C₄-C₂₀ cycloalkylalkyl, aryl, arylalkyl, a group bearing an acidic moiety and a group bearing an amino moiety, each of which may be further optionally substituted with functional groups which allow conjugation with a suitable molecule able to interact with physiological systems.

Functional groups which allow conjugation with targeting molecules or other molecules that are able to interact with physiological systems are known to those skilled in the art. Such groups include, for example, carboxylic acids, amines, aldehydes, alkyl halogens, alkyl maleimides, sulfhydryl groups, hydroxyl groups, etc.

Specific examples of such Aazta metal chelators or derivatives thereof include, but are not limited, to CyAazta. Aazta derivatives also include:

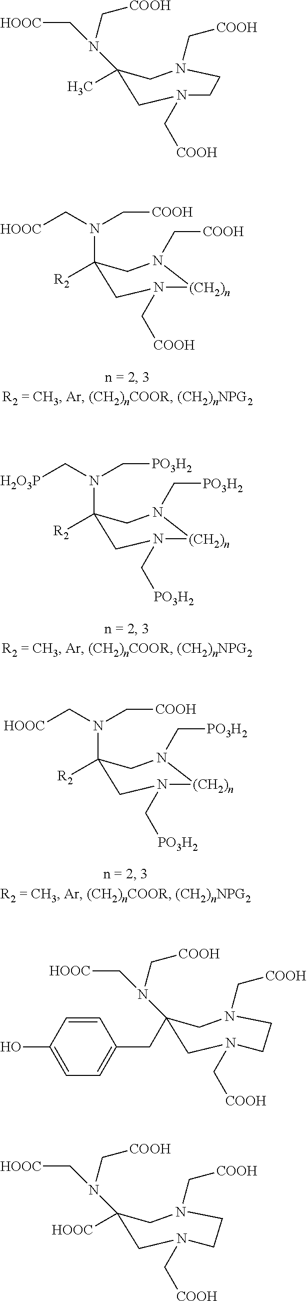

In a preferred embodiment, the metal chelator includes cyclic or acyclic polyaminocarboxylic acids such as DOTA (1,4,7,10-tetraazacyclododecane-1,4,7,10-tetraacetic acid), DTPA (diethylenetriaminepentaacetic acid), DTPA-bismethylamide, DTPA-bismorpholineamide, Cm4 pm 10 d2a (1,4-carbonylmethyl-10-phosphonomethyl-1,4,7,10-Tetraazacyclododecane-1,7-diacetic acid), DO3A N-[[4,7,10-Tris(carboxymethyl)-1,4,7,10-tetraazacyclododec-1-yl]acetyl, HP-DO3A, DO3A-monoamide and derivatives thereof. In another preferred embodiment, the metal chelator includes Aazta or a derivative thereof.

Preferred metal radionuclides for scintigraphy or radiotherapy include ^{99m}Tc, ⁵¹Cr, ⁶⁷Ga, ⁶⁸Ga, ⁴⁷Sc, ⁵¹Cr, ¹⁶⁷Tm, ¹⁴¹Ce, ¹¹¹In, ¹⁶⁸Yb, ¹⁷⁵Yb, ¹⁴⁰La, ⁹⁰Y, ⁸⁸Y, ¹⁵³Sm, ¹⁶⁶Ho, ¹⁶⁵Dy, ¹⁶⁶Dy, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ^{117m}Sn, ¹⁴⁹Pm, ¹⁶¹Tb, ¹⁷⁷Lu, ¹⁹⁸Au and ¹⁹⁹Au and oxides or nitrides thereof. The choice of metal will be determined based on the desired therapeutic or diagnostic application. For example, for diagnostic purposes (e.g., to diagnose and monitor therapeutic progress in primary tumors and metastases), the preferred radionuclides include ⁶⁴Cu, ⁶⁷Ga, ⁶⁸Ga, ^{99m}Tc, and ¹¹¹In, with ^{99m}Tc, ¹¹¹In and ⁶⁸Ga being especially preferred. For therapeutic purposes (e.g., to provide radiotherapy for primary tumors and metastasis related to cancers of the prostate, breast, lung, etc.), the preferred radionuclides include ⁶⁴Cu, ⁹⁰Y, ¹⁰⁵Rh, ¹¹¹In, ^{117m}Sn, ¹⁴⁹Pm, ¹⁵³Sm, ¹⁶¹Tb, ¹⁶⁶Dy, ¹⁶⁶Ho, ¹⁷⁵Yb, ¹⁷⁷Lu, ^{186/188}Re, and ¹⁹⁹Au, with ¹⁷⁷Lu and ⁹⁰Y being particularly preferred.

^{99m}Tc is particularly useful and is a preferred for diagnostic radionuclide for SPECT and planar imaging because of its low cost, availability, imaging properties, and high specific activity. The nuclear and radioactive properties of ^{99m}Tc make this isotope an ideal scintigraphic imaging agent. This isotope has a single photon energy of 140 keV and a radioactive half-life of about 6 hours, and is readily available from a ⁹⁹Mo-^{99m}Tc generator. For example, the ^{99m}Tc labeled peptide can be used to diagnose and monitor therapeutic progress in primary tumors and metastases.

Likewise, ⁶⁸Ga is particularly useful as it is an ideal isotope for positron emission tomography (PET). It is produced from a ⁶⁸Germanium/⁶⁸Gallium generator, thus allowing the use of a positron-emitting isotope without access to a cyclotron. Several types of ⁶⁸Ge/⁶⁸Ga generators are known to those skilled in the art. These differ in the nature of the adsorbent used to retain ⁶⁸Ge, the long-lived parent isotope, on the generator and the eluant used to elute the ⁶⁸Ga off of the column (see e.g. Fania et al, Contrast Media Mol. Imaging. 2008, 3 67-77; Zhernosekov et al. J. Nucli Med, 2007, 48, 1741-1748).

Large volumes of acid such as HCl are frequently used to elute such generators, and it is known to those skilled in the art that the high volume and acidity of this eluant is not ideal for subsequent labeling reactions. Therefore, in some cases, the generator eluant is prepurified using, for example, anionic or cationic exchange resins that serve to pre-purify the eluant by removing ⁶⁸Ge breakthrough, and/or to concentrate the ⁶⁸Ga, which can subsequently be removed from the resin using a small volume of acid or a mixture of acid and an organic solvent such as acetone. Alternatively, only a small fraction of the generator eluant, containing the highest concentration of isotope, may be used, a procedure known as fractionation. ⁶⁸Ga has a physical half-life of 68 min, which is compatible with the clearance pharmacokinetics of many low molecular weight radiopharmaceuticals such as peptides, antibody fragments, oligonucleotides and aptamers and small molecules. About 89% of ⁶⁸Ga decays by positron emission and 11% via electron capture. The average positron energy per disintegration is 740 keV, an energy that is useful for PET imaging.

⁶⁸Ga labeled compounds are typically prepared in a hot cell using automated synthesizers that can be programmed to prepare and (if needed) purify the compound remotely; this protects the chemist from undue radiation exposure. Due to the short half-life of the isotope, methods useful for speeding up reaction rates, including the use of microwave heating, can be advantageous (Velikyan, WO2004/089517).

GRP-containing peptides labeled with ¹⁷⁷Lu, ⁹⁰Y or other therapeutic radionuclides can be used to provide radiotherapy for primary tumors and metastasis related to cancers of the prostate, breast, lung, etc.

### 1B. Optical Labels

In an exemplary embodiment, the compounds of the invention may be conjugated with photolabels, such as optical dyes, including organic chromophores or fluorophores, having extensive delocalized ring systems and having absorption or emission maxima in the range of 400-1500 nm. The compounds of the invention may alternatively be derivatized with a bioluminescent molecule. The preferred range of absorption maxima for photolabels is between 600 and 1000 nm to minimize interference with the signal from hemoglobin. Preferably, photoabsorption labels have large molar absorptivities, e.g. >10⁵ cm⁻¹M⁻¹, while fluorescent optical dyes will have high quantum yields. Examples of optical dyes include, but are not limited to those described in WO 98/18497, WO 98/18496, WO 98/18495, WO 98/18498, WO 98/53857, WO 96/17628, WO 97/18841, WO 96/23524, WO 98/47538, and references cited therein. For example, the photolabels may be covalently linked directly to compounds of the invention, such as, for example, compounds comprised of GRP receptor targeting peptides and linkers of the invention. Several dyes that absorb and emit light in the visible and near-infrared region of electromagnetic spectrum are currently being used for various biomedical applications due to their biocompatibility, high molar absorptivity, and/or high fluorescence quantum yields. The high sensitivity of the optical modality in conjunction with dyes as contrast agents parallels that of nuclear medicine, and permits visualization of organs and tissues without the undesirable effect of ionizing radiation. Cyanine dyes with intense absorption and emission in the near-infrared (NIR) region are particularly useful because biological tissues are optically transparent in this region. For example, Indocyanine green, which absorbs and emits in the NIR region has been used for monitoring cardiac output, hepatic functions, and liver blood flow and its functionalized derivatives have been used to conjugate biomolecules for diagnostic purposes (R. B. Mujumdar, L. A. Ernst, S. R. Mujumdar, et al., Cyanine dye labeling reagents: Sulfoindocyanine succinimidyl esters. Bioconjugate Chemistry, 1993, 4(2), 105-111; Linda G. Lee and Sam L. Woo. “N-Heteroaromatic ion and iminium ion substituted cyanine dyes for use as fluorescent labels”, U.S. Pat. No. 5,453,505; Eric Hohenschuh, et al. “Light imaging contrast agents”, WO 98/48846; Jonathan Turner, et al. “Optical diagnostic agents for the diagnosis of neurodegenerative diseases by means of near infra-red radiation”, WO 98/22146; Kai Licha, et al. “In-vivo diagnostic process by near infrared radiation”, WO 96/17628; Robert A. Snow, et al., Compounds, WO 98/48838. Various imaging techniques and reagents are described in U.S. Pat. Nos. 6,663,847, 6,656,451, 6,641,798, 6,485,704, 6,423,547, 6,395,257, 6,280,703, 6,277,841, 6,264,920, 6,264,919, 6,228,344, 6,217,848, 6,190,641, 6,183,726, 6,180,087, 6,180,086, 6,180,085, 6,013,243, and published U.S. Patent Applications 2003185756, 20031656432, 2003158127, 2003152577, 2003143159, 2003105300, 2003105299, 2003072763, 2003036538, 2003031627, 2003017164, 2002169107, 2002164287, and 2002156117. All of the above references are incorporated by reference in their entirety.

### 1C. Moieties That Contain a Radiolabeled Halogen

Moieties that contain a radiolabeled halogen, such as, for example radioactive iodine, fluorine, etc. are known in the art, as are methods of attaching them to the N—O—P-G components of the invention. In a preferred embodiment moieties such as those disclosed in Zhang et al, J. Nuclear Medicine 47:492-501 (2006) are employed.

### 2A. Linkers Containing at Least One Non-Alpha Amino Acid

In one embodiment of the invention, the linker N—O—P contains at least one non-alpha amino acid. Thus, in this embodiment of the linker N—O—P,
N is 0 (where 0 means it is absent), an alpha or non-alpha amino acid or other linking group;
O is an alpha or non-alpha amino acid; and
P is 0, an alpha or non-alpha amino acid or other linking group,
wherein at least one of N, O or P is a non-alpha amino acid.
   Thus, in one example, N=Gly, O=a non-alpha amino acid, and P=0.

Alpha amino acids are well known in the art, and include naturally occurring and synthetic amino acids.

Non-alpha amino acids are also known in the art and include those which are naturally occurring or synthetic. Preferred non-alpha amino acids include:
8-amino-3,6-dioxaoctanoic acid;
N-4-aminoethyl-N-1-acetic acid; and
   polyethylene glycol derivatives having the formula NH₂—(CH₂CH₂O)n-CH₂CO₂H or NH₂—(CH₂CH₂O)n-CH₂CH₂CO₂H where n=2 to 100.

Examples of compounds having the formula M-N—O—P-G which contain linkers with at least one non-alpha amino acid are listed in Table 1. These compounds may be prepared using the methods disclosed herein, particularly in the Examples, as well as by similar methods known to one skilled in the art.

TABLE 1
Compounds Containing Linkers With At Least One Non-alpha Amino Acid

| | HPLC | HPLC | | | | | | | |
|---|---|---|---|---|---|---|---|---|---|
| Compound | method¹ | RT² | MS³ | IC₅₀⁵ | M | N | O | P | G |
| L1 | 10-40% B | 5.43 | 1616.6 | 5 | N,N- | Lys | 8-amino- | none | BBN(7-14 [symbol] |
| | | | | | dimethylglycine-Ser- | | 3,6- | | |
| | | | | | Cys(Acm)-Gly | | dioxaoctanoic | | |
| | | | | | | | acid | | |
| L2 | 10-40% B | 5.47 | 1644.7 | 3 | N,N- | Arg | 8-amino- | none | BBN(7-14 [symbol] |
| | | | | | dimethylglycine-Ser- | | 3,6- | | |
| | | | | | Cys(Acm)-Gly | | dioxaoctanoic | | |
| | | | | | | | acid | | |
| L3 | 10-40% B | 5.97 | 1604.6 | >50 | N,N- | Asp | 8-amino- | none | BBN(7-14 [symbol] |
| | | | | | dimethylglycine-Ser- | | 3,6- | | |
| | | | | | Cys(Acm)-Gly | | dioxaoctanoic | | |
| | | | | | | | acid | | |
| L4 | 10-40% B | 5.92 | 1575.5 | 4 | N,N- | Ser | 8-amino- | none | BBN(7-14 [symbol] |
| | | | | | dimethylglycine-Ser- | | 3,6- | | |
| | | | | | Cys(Acm)-Gly | | dioxaoctanoic | | |
| | | | | | | | acid | | |
| L5 | 10-40% B | 5.94 | 1545.5 | 9 | N,N- | Gly | 8-amino- | none | BBN(7-14 [symbol] |
| | | | | | dimethylglycine-Ser- | | 3,6- | | |
| | | | | | Cys(Acm)-Gly | | dioxaoctanoic | | |
| | | | | | | | acid | | |
| L6 | 10-30% B | 7.82 | 1639 | >50 | N,N- | Glu | 8-amino- | none | BBN(7-14 [symbol] |
| | | | (M + Na) | | dimethylglycine-Ser- | | 3,6- | | |
| | | | | | Cys(Acm)-Gly | | dioxaoctanoic | | |
| | | | | | | | acid | | |
| L7 | 10-30% B | 8.47 | 1581 | 7 | N,N- | Dala | 8-amino- | none | BBN(7-14 [symbol] |
| | | | (M + Na) | | dimethylglycine-Ser- | | 3,6- | | |
| | | | | | Cys(Acm)-Gly | | dioxaoctanoic | | |
| | | | | | | | acid | | |
| L8 | 10-30% B | 6.72 | 1639 | 4 | N,N- | 8-amino-3,6- | Lys | none | BBN(7-14 [symbol] |
| | | | (M + Na) | | dimethylglycine-Ser- | dioxaoctanoic | | | |
| | | | | | Cys(Acm)-Gly | acid | | | |
| L9 | 10-30% B | 7.28 | 823.3 | 6 | N,N- | 8-amino-3,6- | Arg | none | BBN(7-14 [symbol] |
| | | | (M + Na) | | dimethylglycine-Ser- | dioxaoctanoic | | | |
| | | | | | Cys(Acm)-Gly | acid | | | |
| L10 | 10-30% B | 7.94 | 1625.6 | >50 | N,N- | 8-amino-3,6- | Asp | none | BBN(7-14 [symbol] |
| | | | (M + Na) | | dimethylglycine-Ser- | dioxaoctanoic | | | |
| | | | | | Cys(Acm)-Gly | acid | | | |
| L11 | 10-30% B | 7.59 | 1575.6 | 36 | N,N- | 8-amino-3,6- | Ser | none | BBN(7-14 [symbol] |
| | | | | | dimethylglycine-Ser- | dioxaoctanoic | | | |
| | | | | | Cys(Acm)-Gly | acid | | | |
| L12 | 10-30% B | 7.65 | 1567.5 | >50 | N,N- | 8-amino-3,6- | Gly | none | BBN(7-14 [symbol] |
| | | | (M + Na) | | dimethylglycine-Ser- | dioxaoctanoic | | | |
| | | | | | Cys(Acm)-Gly | acid | | | |
| L13 | 10-30% B | 7.86 | 1617.7 | >50 | N,N- | 8-amino-3,6- | Glu | none | BBN(7-14 [symbol] |
| | | | | | dimethylglycine-Ser- | dioxaoctanoic | | | |
| | | | | | Cys(Acm)-Gly | acid | | | |
| L14 | 10-30% B | 7.9 | 1581.7 | 11 | N,N- | 8-amino-3,6- | Dala | none | BBN(7-14 [symbol] |
| | | | (M + Na) | | dimethylglycine-Ser- | dioxaoctanoic | | | |
| | | | | | Cys(Acm)-Gly | acid | | | |
| L15 | 10-30% B | 7.84 | 1656.8 | 11.5 | N,N- | 8-amino-3,6- | 8-amino- | none | BBN(7-14 [symbol] |
| | | | (M + Na) | | dimethylglycine-Ser- | dioxaoctanoic | 3,6- | | |
| | | | | | Cys(Acm)-Gly | acid | dioxaoctanoic | | |
| | | | | | | | acid | | |
| L16 | 10-30% B | 6.65 | 1597.4 | 17 | N,N- | 8-amino-3,6- | 2,3- | none | BBN(7-14 [symbol] |
| | | | (M + Na) | | dimethylglycine-Ser- | dioxaoctanoic | diaminopropionic | | |
| | | | | | Cys(Acm)-Gly | acid | acid | | |
| L17 | 10-30% B | 7.6 | 1488.6 | 8 | N,N- | none | 8-amino- | none | BBN(7-14 [symbol] |
| | | | | | dimethylglycine-Ser- | | 3,6- | | |
| | | | | | Cys(Acm)-Gly | | dioxaoctanoic | | |
| | | | | | | | acid | | |
| L18 | 10-30% B | 7.03 | 1574.6 | 7.8 | N,N- | 2,3- | 8-amino- | none | BBN(7-14 [symbol] |
| | | | | | dimethylglycine-Ser- | diaminopropionic | 3,6- | | |
| | | | | | Cys(Acm)-Gly | acid | dioxaoctanoic | | |
| | | | | | | | acid | | |
| L19 | 10-35% B | 5.13 | 1603.6 | >50 | N,N- | Asp | 8-amino- | Gly | BBN(7-14 [symbol] |
| | | | | | dimethylglycine-Ser- | | 3,6- | | |
| | | | | | Cys(Acm)-Gly | | dioxaoctanoic | | |
| | | | | | | | acid | | |
| L20 | 10-35% B | 5.19 | 1603.6 | 37 | N,N- | 8-amino-3,6- | Asp | Gly | BBN(7-14 [symbol] |
| | | | | | dimethylglycine-Ser- | dioxaoctanoic | | | |
| | | | | | Cys(Acm)-Gly | acid | | | |
| L21 | 10-35% B | 5.04 | 1575.7 | 46 | N,N- | 8-amino-3,6- | Ser | Gly | BBN(7-14 [symbol] |
| | | | | | dimethylglycine-Ser- | dioxaoctanoic | | | |
| | | | | | Cys(Acm)-Gly | acid | | | |
| L22 | 10-35% B | 4.37 | 1644.7 | 36 | N,N- | 8-amino-3,6- | Arg | Gly | BBN(7-14 [symbol] |
| | | | | | dimethylglycine-Ser- | dioxaoctanoic | | | |
| | | | | | Cys(Acm)-Gly | acid | | | |
| L23 | 10-35% B | 5.32 | 1633.7 | >50 | N,N- | 8-amino-3,6- | 8-amino- | Gly | BBN(7-14 [symbol] |
| | | | | | dimethylglycine-Ser- | dioxaoctanoic | 3,6- | | |
| | | | | | Cys(Acm)-Gly | acid | dioxaoctanoic | | |
| | | | | | | | acid | | |
| L24 | 10-35% B | 4.18 | 1574.6 | 38 | N,N- | 8-amino-3,6- | 2,3- | Gly | BBN(7-14 [symbol] |
| | | | | | dimethylglycine-Ser- | dioxaoctanoic | diaminopropionic | | |
| | | | | | Cys(Acm)-Gly | acid | acid | | |
| L25 | 10-35% B | 4.24 | 1616.6 | 26 | N,N- | 8-amino-3,6- | Lys | Gly | BBN(7-14 [symbol] |
| | | | | | dimethylglycine-Ser- | dioxaoctanoic | | | |
| | | | | | Cys(Acm)-Gly | acid | | | |
| L26 | 10-35% B | 4.45 | 1574.6 | 30 | N,N- | 2,3- | 8-amino- | Gly | BBN(7-14 [symbol] |
| | | | | | dimethylglycine-Ser- | diaminopropionic | 3,6- | | |
| | | | | | Cys(Acm)-Gly | acid | dioxaoctanoic | | |
| | | | | | | | acid | | |
| L27 | 10-35% B | 4.38 | 1627.3 | >50 | N,N- | N-4-aminoethyl- | Asp | none | BBN(7-14 [symbol] |
| | | | | | dimethylglycine-Ser- | N-1- | | | |
| | | | | | Cys(Acm)-Gly | piperazineacetic | | | |
| | | | | | | acid | | | |
| L28 | 10-35% B | 4.1 | 1600.3 | 25 | N,N- | N-4-aminoethyl- | Ser | none | BBN(7-14 [symbol] |
| | | | | | dimethylglycine-Ser- | N-1- | | | |
| | | | | | Cys(Acm)-Gly | piperazineacetic | | | |
| | | | | | | acid | | | |
| L29 | 10-35% B | 3.71 | 1669.4 | 36 | N,N- | N-4-aminoethyl- | Arg | none | BBN(7-14 [symbol] |
| | | | | | dimethylglycine-Ser- | N-1- | | | |
| | | | | | Cys(Acm)-Gly | piperazineacetic | | | |
| | | | | | | acid | | | |
| L30 | 10-35% B | 4.57 | 1657.2 | 36 | N,N- | N-4-aminoethyl- | 8-amino- | none | BBN(7-14 [symbol] |
| | | | | | dimethylglycine-Ser- | N-1- | 3,6- | | |
| | | | | | Cys(Acm)-Gly | piperazineacetic | dioxaoctanoic | | |
| | | | | | | acid | acid | | |
| L31 | 10-35% B | 3.69 | 1598.3 | >50 | N,N- | N-4-aminoethyl- | 2,3- | none | BBN(7-14 [symbol] |
| | | | | | dimethylglycine-Ser- | N-1- | diaminopropionic | | |
| | | | | | Cys(Acm)-Gly | piperazineacetic | acid | | |
| | | | | | | acid | | | |
| L32 | 10-35% B | 3.51 | 1640.3 | 34 | N,N- | N-4-aminoethyl- | Lys | none | BBN(7-14 [symbol] |
| | | | | | dimethylglycine-Ser- | N-1- | | | |
| | | | | | Cys(Acm)-Gly | piperazineacetic | | | |
| | | | | | | acid | | | |
| L33 | 10-35% B | 4.29 | 1584.5 | >50 | N,N- | N-1- | Asp | none | BBN(7-14 [symbol] |
| | | | | | dimethylglycine-Ser- | piperazineacetic | | | |
| | | | | | Cys(Acm)-Gly | acid | | | |
| L34 | 10-35% B | 4.07 | 1578.7 | 38 | N,N- | N-1- | Ser | none | BBN(7-14 [symbol] |
| | | | (M + Na) | | dimethylglycine-Ser- | piperazineacetic | | | |
| | | | | | Cys(Acm)-Gly | acid | | | |
| L35 | 10-35% B | 3.65 | 1625.6 | 26 | N,N- | N-1- | Arg | none | BBN(7-14 [symbol] |
| | | | | | dimethylglycine-Ser- | piperazineacetic | | | |
| | | | | | Cys(Acm)-Gly | acid | | | |
| L36 | 10-35% B | 4.43 | 1636.6 | 7 | N,N- | N-1- | 8-amino- | none | BBN(7-14 [symbol] |
| | | | | | dimethylglycine-Ser- | piperazineacetic | 3,6- | | |
| | | | | | Cys(Acm)-Gly | acid | dioxaoctanoic | | |
| | | | | | | | acid | | |
| L37 | 10-35% B | 3.66 | 1555.7 | 23 | N,N- | N-1- | 2,3- | none | BBN(7-14 [symbol] |
| | | | | | dimethylglycine-Ser- | piperazineacetic | diaminopropionic | | |
| | | | | | Cys(Acm)-Gly | acid | acid | | |
| L38 | 10-35% B | 3.44 | 1619.6 | 7 | N,N- | N-1- | Lys | none | BBN(7-14 [symbol] |
| | | | | | dimethylglycine-Ser- | piperazineacetic | | | |
| | | | | | Cys(Acm)-Gly | acid | | | |
| L42 | 30-50% B | 5.65 | 1601.6 | 25 | N,N- | 4- | 8-amino- | none | BBN(7-14 [symbol] |
| | | | | | dimethylglycine-Ser- | Hydroxyproline | 3,6- | | |
| | | | | | Cys(Acm)-Gly | | dioxaoctanoic | | |
| | | | | | | | acid | | |
| L48 | 30-50% B | 4.47 | 1600.5 | 40 | N,N- | 4-aminoproline | 8-amino- | none | BBN(7-14 [symbol] |
| | | | | | dimethylglycine-Ser- | | 3,6- | | |
| | | | | | Cys(Acm)-Gly | | dioxaoctanico | | |
| | | | | | | | acid | | |
| L51 | 15-35% B | 5.14 | 1673.7 | 49 | N,N- | Lys | 8-amino- | Gly | BBN(7-14 [symbol] |
| | | | | | dimethylglycine-Ser- | | 3,6- | | |
| | | | | | Cys(Acm)-Gly | | dioxaoctanoic | | |
| | | | | | | | acid | | |
| L52 | 15-35% B | 6.08 | 1701.6 | 14 | N,N- | Arg | 8-amino- | Gly | BBN(7-14 [symbol] |
| | | | | | dimethylglycine-Ser- | | 3,6- | | |
| | | | | | Cys(Acm)-Gly | | dioxaoctanoic | | |
| | | | | | | | acid | | |
| L53 | 15-35% B | 4.16 | 1632.6 | 10 | N,N- | Ser | 8-amino- | Gly | BBN(7-14 [symbol] |
| | | | | | dimethylglycine-Ser- | | 3,6- | | |
| | | | | | Cys(Acm)-Gly | | dioxaoctanoic | | |
| | | | | | | | acid | | |
| L54 | 15-35% B | 4.88 | 1661.6 | >50 | N,N- | Asp | 8-amino- | Gly | BBN(7-14 [symbol] |
| | | | | | dimethylglycine-Ser- | | 3,6- | | |
| | | | | | Cys(Acm)-Gly | | dioxaoctanoic | | |
| | | | | | | | acid | | |
| L55 | 15-35% B | 4.83 | 1683.4 | 43 | N,N- | 8-amino-3,6- | Asp | Gly | BBN(7-14 [symbol] |
| | | | (M + Na) | | dimethylglycine-Ser- | dioxaoctanoic | | | |
| | | | | | Cys(Acm)-Gly | acid | | | |
| L56 | 15-35% B | 4.65 | 1655.7 | 4 | N,N- | 8-amino-3,6- | Ser | Gly | BBN(7-14 [symbol] |
| | | | (M + Na) | | dimethylglycine-Ser- | dioxaoctanoic | | | |
| | | | | | Cys(Acm)-Gly | acid | | | |
| L57 | 15-35% B | 4.9 | 1701.8 | >50 | N,N- | 8-amino-3,6- | Arg | Gly | BBN(7-14 [symbol] |
| | | | | | dimethylglycine-Ser- | dioxaoctanoic | | | |
| | | | | | Cys(Acm)-Gly | acid | | | |
| L58 | 15-35% B | 4.22 | 846.4 | >50 | N,N- | 8-amino-3,6- | 8-amino- | Gly | BBN(7-14 [symbol] |
| | | | (M + Ha) | | dimethylglycine-Ser- | dioxaoctanoic | 3,6- | | |
| | | | | | Cys(Acm)-Gly | acid | dioxaoctanoic | | |
| | | | | | | | acid | | |
| L59 | 15-35% B | 4.03 | 1635.5 | 42 | N,N- | 8-amino-3,6- | 2,3- | Gly | BBN(7-14 [symbol] |
| | | | | | dimethylglycine-Ser- | dioxaoctanoic | diaminopropionic | | |
| | | | | | Cys(Acm)-Gly | acid | acid | | |
| L60 | 15-35% B | 4.11 | 1696.6 | 20 | N,N- | 8-amino-3,6- | Lys | Gly | BBN(7-14 [symbol] |
| | | | (M + Na) | | dimethylglycine-Ser- | dioxaoctanoic | | | |
| | | | | | Cys(Acm)-Gly | acid | | | |
| L61 | 15-35% B | 4.32 | 1631.4 | 43 | N,N- | 2,3- | 8-amino- | Gly | BBN(7-14 [symbol] |
| | | | | | dimethylglycine-Ser- | diaminopropionic | 3,6- | | |
| | | | | | Cys(Acm)-Gly | acid | dioxaoctanoic | | |
| | | | | | | | acid | | |
| L78 | 20-40% B | 6.13 | 1691.4 | 35 | DO3A-monoamide | 8-amino-3,6- | Diaminopropionic | none | BBN(7-14 [symbol] |
| | | | (M + Na) | | | dioxaoctanoic | acid | | |
| | | | | | | acid | | | |
| L79 | 20-40% B | 7.72 | 1716.8 | 42 | DO3A-monoamide | 8-amino-3,6- | Biphenylalanine | none | BBN(7-14 [symbol] |
| | | | (M + Na) | | | dioxaoctanoic | | | |
| | | | | | | acid | | | |
| L80 | 20-40% B | 7.78 | 1695.9 | >50 | DO3A-monoamide | 8-amino-3,6- | Diphenylalanine | none | BBN(7-14 [symbol] |
| | | | | | | dioxaoctanoic | | | |
| | | | | | | acid | | | |
| L81 | 20-40% B | 7.57 | 1513.6 | 37.5 | DO3A-monoamide | 8-amino-3,6- | 4- | none | BBN(7-14 [symbol] |
| | | | | | | dioxaoctanoic | Benzoylphe- | | |
| | | | | | | acid | nylalanine | | |
| L92 | 15-30% B | 5.63 | 1571.6 | 5 | DO3A-monoamide | 5- | 8-amino- | none | BBN(7-14 [symbol] |
| | | | | | | aminopentanoic | 3,6- | | |
| | | | | | | acid | dioxaoctanoic | | |
| | | | | | | | acid | | |
| L94 | 20-36% B | 4.19 | 1640.8 | 6.2 | DO3A-monoamide | 8-amino-3,6- | D- | none | BBN(7-14 [symbol] |
| | | | (M + Na) | | | dioxaoctanoic | Phenylalanine | | |
| | | | | | | acid | | | |
| L110 | 15-45% B | 5.06 | 1612.7 | 36 | DO3A-monoamide | 8- | 8-amino- | none | BBN(7-14 [symbol] |
| | | | | | | aminooctanoic | 3,6- | | |
| | | | | | | acid | dioxaoctanoic | | |
| | | | | | | | acid | | |
| L209 | 20-40% B | 4.62 | 3072.54 | 37 | DO3A-monoamide | E(G8-amino- | 8- | 8- | BBN(7-14 [symbol] |
| | over 6 | | | | | 3,6- | aminooctanoic | aminooc | |
| | minutes | | | | | dioxaoctanoic | acid | tanoic | |
| | | | | | | acid-8- | | acid | |
| | | | | | | amino-3,6- | | | |
| | | | | | | dioxaoctanoic | | | |
| | | | | | | acid | | | |
| | | | | | | QWAVGHL | | | |
| | | | | | | M-NH₂) | | | |
| L210 | 20-50% B | 6.18 | 3056.76 | 11 | DO3A-monoamide | E(G-Aoa- | 8- | 8- | BBN(7-14 [symbol] |
| | over 10 | | | | | Aoa-QWAVGHL | aminooctanoic | aminooc | |
| | minutes | | | | | M-NH₂) | acid | tanoic | |
| | | | | | | | | acid | |

*BBN(7-14) corresponds to QWAVGHLM,which is (SEQ ID NO:1)
¹HPLC method refers to gradient change that occurs over 10 minute′s time for the HPLC gradient.
²HPLC RT refers to the retention time of the compound in the HPLC.
³MS refers to mass spectra where molecular weight is calculated from mass/unit charge (m/e).
⁴IC₅₀ refers to the concentration of compound to inhibit 50% binding of iodinated bombesin to a GRP receptor on cells. [symbol]
indicates data missing or illegible when filed

2B. Linkers Containing at Least One Substituted Bile Acid

In another embodiment of the present invention, the linker N—O—P contains at least one substituted bile acid. Thus, in this embodiment of the linker N—O—P,
N is 0 (where 0 means it is absent), an alpha amino acid, a substituted bile acid or other linking group;
O is an alpha amino acid or a substituted bile acid; and
P is 0, an alpha amino acid, a substituted bile acid or other linking group,
   wherein at least one of N, O or P is a substituted acid.

Bile acids are found in bile (a secretion of the liver) and are steroids having a hydroxyl group and a five carbon atom side chain terminating in a carboxyl group. In substituted bile acids, at least one atom such as a hydrogen atom of the bile acid is substituted with another atom, molecule or chemical group. For example, substituted bile acids include those having a 3-amino, 24-carboxyl function optionally substituted at positions 7 and 12 with hydrogen, hydroxyl or keto functionality.

Other useful substituted bile acids in the present invention include substituted cholic acids and derivatives thereof. Specific substituted cholic acid derivatives include:
(3β,5β)-3-aminocholan-24-oic acid;
(3β,5β,12α)-3-amino-12-hydroxycholan-24-oic acid;
(3β,5β,7α,12α)-3-amino-7,12-dihydroxycholan-24-oic acid;
Lys-(3,6,9)-trioxaundecane-1,1′-dicarbonyl-3,7-dideoxy-3-aminocholic acid);
(3β,5β,7α)-3-amino-7-hydroxy-12-oxocholan-24-oic acid; and
(3β,5β,7α)-3-amino-7-hydroxycholan-24-oic acid.

Examples of compounds having the formula M-N—O—P-G which contain linkers with at least one substituted bile acid are listed in Table 2. These compounds may be prepared using the methods disclosed herein, particularly in the Examples, as well as by similar methods known to one skilled in the art.

TABLE 2
Compounds Containing Linkers With At Least One Substituted Bile Acid

| | HPLC | HPLC | | | | | | | |
|---|---|---|---|---|---|---|---|---|---|
| Compound | method¹ | RT² | MS³ | IC50⁵ | M | N | O | P | G |
| L62 | 20-80% | 3.79 | 1741.2 | >50 | DO3A- | Gly | (3β,5β)-3- | none | BBN(7-14)* |
| | B | | | | monoamide | | aminocholan-24- | | |
| | | | | | | | oic acid | | |
| L63 | 20-80% | 3.47 | 1757.0 | 23 | DO3A- | Gly | (3β,5β,12α)-3- | none | BBN(7-14)* |
| | B | | | | monoamide | | amino-12- | | |
| | | | | | | | hydroxycholan- | | |
| | | | | | | | 24-oic acid | | |
| L64 | 20-50% | 5.31 | 1773.7 | 8.5 | DO3A- | Gly | (3β,5β,7α,12α)-3- | none | BBN(7-14)* |
| | B | | | | monoamide | | amino-7,12- | | |
| | | | | | | | dihydroxycholan- | | |
| | | | | | | | 24-oic acid | | |
| L65 | 20-80% | 3.57 | 2246.2 | >50 | DO3A- | Gly | Lys-(3,6,9- | Arg | BBN(7-14)* |
| | B | | | | monoamide | | trioxaundecane- | | |
| | | | | | | | 1,11-dicarbonyl- | | |
| | | | | | | | 3,7- | | |
| | | | | | | | dideoxy-3- | | |
| | | | | | | | aminocholic acid) | | |
| L66 | 20-80% | 3.79 | 2245.8 | >50 | DO3A- | Gly | Lys- | Arg | BBN(7-14)* |
| | | | | | monoamide | | (3β,5β,7α,12α)-3- | | |
| | | | | | | | amino-7,12- | | |
| | | | | | | | dihydroxycholan- | | |
| | | | | | | | 24-oic acid-3,6,9- | | |
| | | | | | | | trioxaundecane- | | |
| | | | | | | | 1,11-dicarbonyl | | |
| L67 | 20-80% | 3.25 | 1756.9 | 4.5 | DO3A- | Gly | (3β,5β,7α,12α)-3- | none | BBN(7-14)* |
| | | | | | monoamide | | amino-12- | | |
| | | | | | | | oxacholan-24-oic | | |
| | | | | | | | acid | | |
| L69 | 20-80% | 3.25 | 1861.27 | 8 | DO3A- | 1-amino-3,6- | (3β,5β,7α,12α)-3- | none | BBN(7-14)* |
| | | | | | monoamide | dioxaoctanoic | amino-7,12- | | |
| | | | | | | acid | dihydroxycholan- | | |
| | | | | | | | 24-oic acid | | |
| L280 | — | — | — | — | DO3A- | Gly | 3β,5β 7α,12α)-3- | none | QWAVaHLM- |
| | | | | | monoamide | | amino-7,12- | | NH2 (SEQ ID |
| | | | | | | | dihydroxycholan- | | NO: 14) |
| | | | | | | | 24-oic acid | | |
| L281 | — | — | — | — | DO3A- | Gly | 3β,5β 7α,12α)-3- | f | QWAVGHLM- |
| | | | | | monoamide | | amino-7,12- | | NH2* |
| | | | | | | | dihydroxycholan- | | |
| | | | | | | | 24-oic acid | | |
| L282 | — | — | — | — | DO3A- | Gly | 3β,5β 7α,12α)-3- | f | QWAVGHL-L- |
| | | | | | monoamide | | amino-7,12- | | NH2 (SEQ ID |
| | | | | | | | dihydroxycholan- | | NO: 8) |
| | | | | | | | 24-oic acid | | |
| L283 | — | — | — | — | DO3A- | Gly | 3β,5β 7α,12α)-3- | f | QWAVGHL-N [symbol] |
| | | | | | monoamide | | amino-7,12- | | pentyl |
| | | | | | | | dihydroxycholan- | | (SEQ ID NO: 6) |
| | | | | | | | 24-oic acid | | |
| L284 | — | — | — | — | DO3A- | Gly | 3β,5β 7α,12α)-3- | y | QWAVBala- |
| | | | | | monoamide | | amino-7,12- | | HFNle-NH₂ |
| | | | | | | | dihydroxycholan- | | (SE [symbol] |
| | | | | | | | 24-oic acid | | ID NO: 9) |
| L285 | — | — | — | — | DO3A- | Gly | 3β,5β 7α,12α)-3- | f | QWAVBala- |
| | | | | | monoamide | | amino-7,12- | | HFN1e-NH₂ |
| | | | | | | | dihydroxycholan- | | (SE [symbol] |
| | | | | | | | 24-oic acid | | ID NO; 9) |
| L286 | — | — | — | — | DO3A- | Gly | 3β,5β 7α,12α)-3- | none | QWAVGHFL-NH₂ |
| | | | | | monoamide | | amino-7,12- | | (SEQ ID N [symbol] |
| | | | | | | | dihydroxycholan- | | 22) |
| | | | | | | | 24-oic acid | | |
| L287 | — | — | — | — | DO3A- | Gly | 3β,5β 7α,12α)-3- | none | QWAVGNMeH |
| | | | | | monoamide | | amino-7,12- | | LM-NH₂ |
| | | | | | | | dihydroxycholan- | | (SEQ [symbol] |
| | | | | | | | 24-oic acid | | NO: 15) |
| L288 | — | — | — | — | DO3A- | Gly | 3β,5β 7α,12α)-3- | none | LWAVGSF-M-NH₂ |
| | | | | | monoamide | | amino-7,12- | | (SEQ ID N [symbol] |
| | | | | | | | dihydroxycholan- | | 11) |
| | | | | | | | 24-oic acid | | |
| L289 | — | — | — | — | DO3A- | Gly | 3β,5β 7α,12α)-3- | none | HWAVGHL-M [symbol] |
| | | | | | monoamide | | amino-7,12- | | NH₂ |
| | | | | | | | dihydroxycholan- | | (SEQ ID N [symbol] |
| | | | | | | | 24-oic acid | | NO 12) |
| L290 | — | — | — | — | DO3A- | Gly | 3β,5β 7α,12α)-3- | none | LWATGH-F-M [symbol] |
| | | | | | monoamide | | amino-7,12- | | NH₂ |
| | | | | | | | dihydroxycholan- | | (SEQ ID N [symbol] |
| | | | | | | | 24-oic acid | | NO. 16) |
| L291 | — | — | — | — | DO3A- | Gly | 3β,5β 7α,12α)-3- | none | QWAVGH- |
| | | | | | monoamide | | amino-7,12- | | FMNH₂ |
| | | | | | | | dihydroxycholan- | | (SEQ I [symbol] |
| | | | | | | | 24-oic acid | | NO: 17) |
| L292 | — | — | — | — | DO3A- | Gly | 3β,5β 7α,12α)-3- | QRLGN | QWAVGHLM- |
| | | | | | monoamide | | amino-7,12- | | NH₂* |
| | | | | | | | dihydroxycholan- | | |
| | | | | | | | 24-oic acid | | |
| L293 | — | — | — | — | DO3A- | Gly | 3β,5β 7α,12α)-3- | QRYGN | QWAVGHLM- |
| | | | | | monoamide | | amino-7,12- | | NH₂* |
| | | | | | | | dihydroxycholan- | | |
| | | | | | | | 24-oic acid | | |
| L294 | — | — | — | — | DO3A- | Gly | 3β,5β 7α,12α)-3- | QKYGN | QWAVGHLM- |
| | | | | | monoamide | | amino-7,12- | | NH₂* |
| | | | | | | | dihydroxycholan- | | |
| | | | | | | | 24-oic acid | | |
| L295 | — | — | — | — | Pglu-Q-Lys | Gly | 3β,5β 7α,12α)-3- | LG-N | QWAVGHLM- |
| | | | | | (DO3A- | | amino-7,12- | | NH₂* |
| | | | | | monoamide) | | dihydroxycholan- | | |
| | | | | | | | 24-oic acid | | |
| L303 | — | — | — | — | DO3A- | Gly | 3-amino-3- | none | QRLGNQWAV [symbol] |
| | | | | | monoamide | | deoxycholic acid | | HLM-NH₂ |
| | | | | | | | | | (SE [symbol] |
| | | | | | | | | | ID NO: 3) |
| L304 | — | — | — | — | DO3A- | Gly | 3-amino-3- | none | QRYGNQWA [symbol] |
| | | | | | monoamide | | deoxycholic acid | | HLM-NH₂ |
| | | | | | | | | | (SE [symbol] |
| | | | | | | | | | ID NO: 4) |
| L305 | — | — | — | — | DO3A- | Gly | 3-amino-3- | none | QKYGNQWA [symbol] |
| | | | | | monoamide | | deoxycholic acid | | HLM-NH₂ |
| | | | | | | | | | (SE [symbol] |
| | | | | | | | | | ID NO: 5) |
| L306 | — | — | — | — | DO3A- | Gly | 3-amino-3- | none | LGNQWAVGH |
| | | | | | monoamide | | deoxycholic acid | | M-NH₂ |
| | | | | | | | | | (SEQ I [symbol] |
| | | | | | | | | | NO: 18) |
| L502 | — | — | — | — | Aazta | Gly | (3β,5β,7α,12α)- | none | BBN(7-14)* |
| | | | | | | | 3-amino-7,12- | | |
| | | | | | | | dihydroxycholan- | | |
| | | | | | | | 24-oic acid | | |
| L503 | — | — | — | — | CyAazta | Gly | (3β,5β,7α,12α)- | none | BBN(7-14)* |
| | | | | | | | 3-amino-7,12- | | |
| | | | | | | | dihydroxycholan- | | |
| | | | | | | | 24-oic acid | | |

*BBN(7-14) corresponds to QWAVGHLM, which is SEQ ID NO: 1
¹HPLC method refers to the 10 minute time for the HPLC gradient.
²HPLC RT refers to the retention time of the compound in the HPLC.
³MS refers to mass spectra where molecular weight is calculated from mass/unit charge (m/e).
⁴IC₅₀ refers to the concentration of compound to inhibit 50% binding of iodinated bombesin (¹²⁵I-[Tyr⁴]-BBN)to a GRP receptor on cells. [symbol]
indicates data missing or illegible when filed

2C. Linkers Containing at Least One Non-Alpha Amino Acid with a Cyclic Group

In yet another embodiment of the present invention, the linker N—O—P contains at least one non-alpha amino acid with a cyclic group. Thus, in this embodiment of the linker N—O—P,

N is 0 (where 0 means it is absent), an alpha amino acid, a non-alpha amino acid with a cyclic group or other linking group;

O is an alpha amino acid or a non-alpha amino acid with a cyclic group; and

P is 0, an alpha amino acid, a non-alpha amino acid with a cyclic group, or other linking group,

wherein at least one of N, O or P is a non-alpha amino acid with a cyclic group.

Non-alpha amino acids with a cyclic group include substituted phenyl, biphenyl, cyclohexyl or other amine and carboxyl containing cyclic aliphatic or heterocyclic moieties. Examples of such include:
4-aminobenzoic acid (hereinafter referred to as “Abz4 in the specification”)
3-aminobenzoic acid
4-aminomethyl benzoic acid
8-aminooctanoic acid trans-4-aminomethylcyclohexane carboxylic acid
4-(2-aminoethoxy)benzoic acid isonipecotic acid
2-aminomethylbenzoic acid
4-amino-3-nitrobenzoic acid
4-(3-carboxymethyl-2-keto-1-benzimidazolyl-piperidine
6-(piperazin-1-yl)-4-(3H)-quinazolinone-3-acetic acid
(2S,5S)-5-amino-1,2,4,5,6,7-hexahydro-azepino[3,21-hi]indole-4-one-2-carboxylic acid
(4S,7R)-4-amino-6-aza-5-oxo-9-thiabicyclo[4.3.0]nonane-7-carboxylic acid
   3-carboxymethyl-1-phenyl-1,3,8-triazaspiro[4.5]decan-4-one
N1-piperazineacetic acid
N4-aminoethyl-N-1-piperazineacetic acid
(3S)-3-amino-1-carboxymethylcaprolactam
(2S,6S,9)-6-amino-2-carboxymethyl-3,8-diazabicyclo-[4,3,0]-nonane-1,4-dione
   3-amino-3-deoxycholic acid
   4-hydroxybenzoic acid
   4-aminophenylacetic acid
   3-hydroxy-4-aminobenzoic acid
   3-methyl-4-aminobenzoic acid
   3-chloro-4-aminobenzoic acid
   3-methoxy-4-aminobenzoic acid
   6-aminonaphthoic acid
N,N′-Bis(2-aminoethyl)-succinamic acid

Examples of compounds having the formula M-N—O—P-G which contain linkers with at least one alpha amino acid with a cyclic group are listed in Table 3. These compounds may be prepared using the methods disclosed herein, particularly in the Examples, as well as by similar methods known to one skilled in the art.

TABLE 3
Compounds Containing Linkers Related To Amino-(Phenyl, Biphenyl, Cycloalkyl Or
Heterocyclic) Carboxylates

| | HPLC | HPLC | | | | | | | |
|---|---|---|---|---|---|---|---|---|---|
| Compound | method¹ | RT² | MS³ | IC50⁵ | M | N | O | P | G |
| L70 | 10-40% | 6.15 | 1502.6 | 5 | DO3A- | Gly | 4-aminobenzoic | none | BBN(7-14) [symbol] |
| | B | | | | monoamide | | acid | | |
| L71 | 20-50% | 14.14 | 59.68 | 7 | DO3A- | none | 4-aminomethyl- | none | BBN(7-14)* |
| | over 30 | | (M + Na) | | monoamide | | benzoic acid | | |
| | minutes | | | | | | | | |
| L72 | 20-50% | 13.64 | 65.73 | 8 | DO3A- | none | trans-4- | none | BBN(7-14)* |
| | over 30 | | (M + K) | | monoamide | | aminomethyl- | | |
| | minutes | | | | | | cyclohexyl | | |
| | | | | | | | carboxylic acid | | |
| L73 | 5-35% | 7.01 | 1489.8 | 5 | DO3A- | none | 4-(2- | none | BBN(7-14)* |
| | | | | | monoamide | | aminoethoxy) | | |
| | | | | | | | benzoic acid | | |
| L74 | 5-35% | 6.49 | 1494.8 | 7 | DO3A- | Gly | isonipecotic | none | BBN(7-14)* |
| | | | | | monoamide | | acid | | |
| L75 | 5-35% | 6.96 | 1458.0 | 23 | DO3A- | none | 2-aminomethyl- | none | BBN(7-14)* |
| | | | | | monoamide | | benzoic | | |
| | | | | | | | acid | | |
| L76 | 5-35% | 7.20] | 1502.7 | 4 | DO3A- | none | 4-aminomethyl- | none | BBN(7-14)* |
| | | | | | monoamide | | 3-nitrobenzoic | | |
| | | | | | | | acid | | |
| L77 | 20-40% | 6.17 | 1691.8 | 17.5 | DO3A- | 8-amino-3,6- | 1- | none | BBN(7-14)* |
| | B | | (M + Na) | | monoamide | dioxaoctanoic | Naphthylalanine | | |
| | | | | | | acid | | | |
| L82 | 20-40% | 6.18 | 1584.6 | 8 | DO3A- | none | 4-(3- | none | BBN(7-14)* |
| | B | | | | monoamide | | carboxymethyl-2- | | |
| | | | | | | | keto-1- | | |
| | | | | | | | benzimidazolyl- | | |
| | | | | | | | piperidine | | |
| L83 | 20-40% | 5.66 | 1597.5 | >50 | DO3A- | none | 6-(piperazin-1- | none | BBN(7-14)* |
| | B | | | | monoamide | | yl)-4-(3H)- | | |
| | | | | | | | quinazolinone-3- | | |
| | | | | | | | acetic acid | | |
| L84 | 20-40% | 6.31 | 1555.5 | >50 | DO3A- | none | (2S,5S)-5-amino- | none | BBN(7-14)* |
| | B | | | | monoamide | | 1,2,4,5,6,7- | | |
| | | | | | | | hexahydro- | | |
| | | | | | | | azepino[3,21- | | |
| | | | | | | | hi]indole- | | |
| | | | | | | | 4-one-2- | | |
| | | | | | | | carboxylic acid | | |
| L85 | 20-40% | 5.92 | 1525.5 | >50 | DO3A- | none | (4S,7R)-4-amino- | none | BBN(7-14)* |
| | B | | | | monoamide | | 6-aza-5-oxo-9- | | |
| | | | | | | | thiabicyclo[4.3.0] | | |
| | | | | | | | nonane-7- | | |
| | | | | | | | carboxylic acid | | |
| L86 | 20-40% B | 6.46 | 1598.6 | >50 | DO3A- | none | N,N- | none | BBN(7-14)* |
| | | | | | monoamide | | dimethylglycine | | |
| L87 | 20-40% B | 5.47 | 1593.8 | >50 | DO3A- | none | 3-carboxymethyl- | none | BBN(7-14)* |
| | | | (M + Na) | | monoamide | | 1-phenyl-1,3,8- | | |
| | | | | | | | triazaspiro[4.5] | | |
| | | | | | | | decan-4-one | | |
| L88 | 20-40% | 3.84 | 1452.7 | >50 | DO3A- | none | N1- | none | BBN(7-14)* |
| | B | | | | monoamide | | piperazineacetic | | |
| | | | | | | | acid | | |
| L89 | 20-40% | 5.68 | 1518.5 | 23 | DO3A- | none | N-4-aminoethyl- | none | BBN(7-14)* |
| | B | | (M + Na) | | monoamide | | N-1-piperazine- | | |
| | | | | | | | acetic acid | | |
| L90 | 20-40% | 7.95 | 1495.4 | 50 | DO3A- | none | (3S)-3-amino-1- | none | BBN(7-14)* |
| | B | | | | monoamide | | carboxymethyl- | | |
| | | | | | | | caprolactam | | |
| L91 | 20-40% | 3.97 | 1535.7 | >50 | DO3A- | none | (2S,6S,9)-6- | none | BBN(7-14)* |
| | B | | | | monoamide | | amino-2- | | |
| | | | | | | | carboxymethyl- | | |
| | | | | | | | 3,8-diazabicyclo- | | |
| | | | | | | | [4,3,0]-nonane- | | |
| | | | | | | | 1,4-dione | | |
| L93 | 15-30% | 7.57 | 1564.7 | 5.8 | DO3A- | 5- | trans-4- | none | BBN(7-14)* |
| | B | | | | monoamide | aminopenta- | aminomethyl- | | |
| | | | | | | noic acid | cyclohexane-1- | | |
| | | | | | | | carboxylic acid | | |
| L95 | 15-35% | 5.41 | 1604.6 | 14 | DO3A- | trans-4- | D- | none | BBN(7-14)* |
| | B | | | | monoamide | aminomethyl- | Phenylalanine | | |
| | | | | | | cyclohexane- | | | |
| | | | | | | 1-carboxylic | | | |
| | | | | | | acid | | | |
| L96 | 20-36% | 4.75 | 1612.7 | 35 | DO3A- | 4- | 8-amino-3,6- | none | BBN(7-14)* |
| | B | | | | monoamide | aminomethyl- | dioxaoctanoic | | |
| | | | | | | benzoic | acid | | |
| | | | | | | acid | | | |
| L97 | 15-35% | 5.86 | 1598.8 | 4.5 | DO3A- | 4- | trans-4- | none | BBN(7-14)* |
| | B | | | | monoamide | benzoyl-(L)- | aminomethyl- | | |
| | | | | | | phenyl- | cyclohexane-l- | | |
| | | | | | | ala nine | carboxylic acid | | |
| L98 | 15-35% | 4.26 | 1622.7 | 16 | DO3A- | trans-4- | Arg | none | BBN(7-14)* |
| | B | | | | monoamide | aminomethyl- | | | |
| | | | | | | cyclo- | | | |
| | | | | | | hexane-1- | | | |
| | | | | | | carboxylic | | | |
| | | | | | | acid | | | |
| L99 | 15-35% | 4.1 | 1594.7 | 22 | DO3A- | trans-4- | Lys | none | BBN(7-14)* |
| | B | | | | monoamide | aminomethyl- | | | |
| | | | | | | cyclo- | | | |
| | | | | | | hexane-1- | | | |
| | | | | | | carboxylic | | | |
| | | | | | | acid | | | |
| L100 | 15-35% | 4.18 | 1613.6 | 10 | DO3A- | trans-4- | Diphenylalanine | none | BBN(7-14)* |
| | B | | | | monoamide | aminomethyl- | | | |
| | | | | | | cyclo- | | | |
| | | | | | | hexane-1- | | | |
| | | | | | | carboxylic | | | |
| | | | | | | acid | | | |
| L101 | 15-35% | 5.25 | 1536.7 | 25 | DO3A- | trans-4- | 1- | none | BBN(7-14)* |
| | B | | | | monoamide | aminomethyl- | Naphthylalanine | | |
| | | | | | | cyclo- | | | |
| | | | | | | hexane-1- | | | |
| | | | | | | carboxylic | | | |
| | | | | | | acid | | | |
| L102 | 15-35% | 5.28 | 1610.8 | 9.5 | DO3A- | trans-4- | 8-amino-3,6- | none | BBN(7-14)* |
| | B | | | | monoamide | aminomethyl- | dioxaoctanoic | | |
| | | | | | | cyclohexane- | acid | | |
| | | | | | | 1-carboxylic | | | |
| | | | | | | acid | | | |
| L103 | 15-35% | 4.75 | 1552.7 | 24 | DO3A- | trans-4- | Ser | none | BBN(7-14)* |
| | B | | | | monoamide | aminomethyl- | | | |
| | | | | | | cyclohexane- | | | |
| | | | | | | 1-carboxylic | | | |
| | | | | | | acid | | | |
| L104 | 15-35% | 3.91 | 1551.7 | 32 | DO3A- | trans-4- | 2,3- | none | BBN(7-14)* |
| | B | | | | monoamide | aminomethyl- | diaminopropionic | | |
| | | | | | | cyclo- | acid | | |
| | | | | | | hexane-1- | | | |
| | | | | | | carboxylic | | | |
| | | | | | | acid | | | |
| L105 | 20-45% | 7.68 | 1689.7 | 3.5 | DO3A- | trans-4- | Biphenylalanine | none | BBN(7-14)* |
| | B | | | | monoamide | aminomethyl- | | | |
| | | | | | | cyclohexane- | | | |
| | | | | | | 1-carboxylic | | | |
| | | | | | | acid | | | |
| L106 | 20-45% | 6.97 | 1662.7 | 3.8 | DO3A- | trans-4- | (2S,5S)-5-amino- | none | BBN(7-14)* |
| | B | | | | monoamide | aminomethyl- | 1,2,4,5,6,7- | | |
| | | | | | | cyclohexane- | hexahydroazepino | | |
| | | | | | | 1-carboxylic | [3,21-hi]indole- | | |
| | | | | | | acid | 4-one-2- | | |
| | | | | | | | carboxylic acid | | |
| L107 | 15-35% | 5.79 | 1604.7 | 5 | DO3A- | trans-4- | trans-4- | none | BBN(7-14)* |
| | B | | | | monoamide | aminomethyl- | aminomethyl- | | |
| | | | | | | cyclohexane- | cyclohexane-1- | | |
| | | | | | | 1-carboxylic | carboxylic acid | | |
| | | | | | | acid | | | |
| L108 | 15-45% | 6.38 | 1618.7 | 10 | DO3A- | 8-amino-3,6- | Phenylalanine | none | BBN(7-14)* |
| | B | | | | monoamide | dioxaocta- | | | |
| | | | | | | noic acid | | | |
| L109 | 15-45% | 6.85 | 1612.7 | 6 | DO3A- | trans-4- | Phenylalanine | none | BBN(7-14)* |
| | B | | | | monoamide | amino- | | | |
| | | | | | | methylcyclo- | | | |
| | | | | | | hexane-1- | | | |
| | | | | | | carboxylic | | | |
| | | | | | | acid | | | |
| L111 | 20-45% | 3.75 | 1628.6 | 8 | DO3A- | 8-amino- | trans-4- | none | BBN(7-14)* |
| | B | | | | monoamide | octanoic | aminomethyl- | | |
| | | | | | | acid | cyclohexane-1- | | |
| | | | | | | | carboxylic acid | | |
| L112 | 20-47% | 3.6 | 1536.5 | 4.5 | DO3A- | none | 4′-aminomethyl- | none | BBN(7-14)* |
| | B in | | | | monoamide | | biphenyl-1- | | |
| | 9 min | | | | | | carboxylic acid | | |
| L113 | 20-47% | 3.88 | 1558.6 | 5 | DO3A- | none | 3′-aminomethyl- | none | BBN(7-14)* |
| | B in | | (M + Na) | | monoamide | | biphenyl-3- | | |
| | 9 min | | | | | | carboxylic acid | | |
| L114 | 10-40% | 5.47 | 1582.8 | 4.5 | CMDOTA | Gly | 4-aminobenzoic | none | BBN(7-14)* |
| | B | | | | | | acid | | |
| L124 | 5-35% | 7.04 | 1489.9 | 8.0 | DO3A- | none | 4-aminomethyl- | none | BBN(7-14)* |
| | B | | | | monoamide | | phenoxyacetic | | |
| | | | | | | | acid | | |
| L143 | 5-35% | 6.85 | 1516.8 | 11 | DO3A- | Gly | 4-amino- | none | BBN(7-14)* |
| | B | | | | monoamide | | phenylacetic | | |
| | | | | | | | acid | | |
| L144 | 5-35% | 6.85 | 1462.7 | 9 | HPDO3A | none | 4-phenoxy | none | BBN(7-14)* |
| | B | | | | | | | | |
| L145 | 20-80% | 1.58 | 1459.8 | 5 | DO3A- | none | 3-aminomethyl- | none | BBN(7-14)* |
| | B | | | | monoamide | | benzoic | | |
| | | | | | | | acid | | |
| L146 | 20-80% | 1.53 | 1473.7 | 9 | DO3A- | none | 4-aminomethyl- | none | BBN(7-14)* |
| | B | | | | monoamide | | phenylacetic | | |
| | | | | | | | acid | | |
| L147 | 20-80% | 1.68 | 1489.7 | 3.5 | DO3A- | none | 4-aminomethyl-3- | none | BBN(7-14)* |
| | B | | | | monoamide | | methoxybenzoic | | |
| | | | | | | | acid | | |
| L201 | 10-46% | 5.77 | 1563.7 | 36 | Boa*** | none | Gly | 4- | BBN(7-14)* |
| | B | | | | | | | amino- | |
| | over 12 | | | | | | | benzoic | |
| | minutes | | | | | | | acid | |
| L202 | 10-46% | 5.68 | 1517.74 | 13 | DO3A- | none | Gly | 4- | BBN(7-14)* |
| | B | | | | monoamide | | | hydra- | |
| | over 12 | | | | | | | zino- | |
| | minutes | | | | | | | benzoyl | |
| L203 | 10-46% | 5.98 | 1444.69 | 9 | DO3A- | none | none | 4- | BBN(7-14)* |
| | B | | | | monoamide | | | amino- | |
| | over 12 | | | | | | | benzoic | |
| | minutes | | | | | | | acid | |
| L204 | 10-46% | 5.82 | 1502.73 | 50 | DO3A- | none | 4-aminobenzoic | Gly | BBN(7-14)* |
| | B | | | | monoamide | | acid | | |
| | over 12 | | | | | | | | |
| | minutes | | | | | | | | |
| L205 | 10-46% | 5.36 | 1503.72 | 45 | DO3A- | Gly | 6-Aminonicotinic | none | BBN(7-14)* |
| | B | | | | monoamide | | acid | | |
| | over 12 | | | | | | | | |
| | minutes | | | | | | | | |
| L206 | 10-46% | 7.08 | 1592.85 | 4.5 | DO3A- | Gly | 4′-Amino-2′- | none | BBN(7-14)* |
| | B | | | | monoamide | | methyl biphenyl- | | |
| | over 12 | | | | | | 4-carboxylic | | |
| | minutes | | | | | | acid | | |
| L207 | 10-46% | 7.59 | 1578.83 | 2.5 | DO3A- | Gly | 3′- | none | BBN(7-14)* |
| | B | | | | monoamide | | Aminobiphenyl- | | |
| | over 12 | | | | | | 3-carboxylic | | |
| | minutes | | | | | | acid | | |
| L208 | 10-46% | 5.9 | 1516.75 | 7.5 | DO3A- | Gly | 1,2-diaminoethyl | Tereph- | BBN(7-14)* |
| | B | | | | monoamide | | | thalic | |
| | over 12 | | | | | | | acid | |
| | minutes | | | | | | | | |
| L211 | 10-46% | 5.76 | 1560.77 | 4 | DO3A- | Gly | Gly | 4- | BBN(7-14)* |
| | B | | | | monoamide | | | amino- | |
| | over 12 | | | | | | | benzoic | |
| | minutes | | | | | | | acid | |
| L212 | 10-46% | 6.05 | 1503.71 | NT** | DO3A- | none | Gly | 4- | EWAVGHL- |
| | B | | | | monoamide | | | amino- | NH₂ (SEQ [symbol] |
| | over 12 | | | | | | | benzoic | NO: 2) |
| | minutes | | | | | | | acid | |
| L213 | 10-46% | 5.93 | 1503.71 | NT** | DO3A- | Gly | 4-aminobenzoic | none | QWAVGH [symbol] |
| | B | | | | monoamide | | acid | | OH* |
| | over 12 | | | | | | | | |
| | minutes | | | | | | | | |
| L214 | 10-46% | 7.36 | 1649.91 | NT** | DO3A- | Gly | 4-aminobenzoic | (D)- | BBN(7-14)* |
| | B | | | | monoamide | | acid | Phe | |
| | over 12 | | | | | | | | |
| | minutes | | | | | | | | |
| L215 | 10-46% | 5.08 | 2071.37 | NT** | DO3A- | Gly | 4-aminobenzoic | none | QRLGNQW |
| | B | | | | monoamide | | acid | | VGHLM-N [symbol] |
| | over 12 | | | | | | | | (SEQ ID N [symbol] |
| | minutes | | | | | | | | 3) |
| L216 | 10-46% | 4.94 | 2121.38 | NT** | DO3A- | Gly | 4-aminobenzoic | none | QRYGNQ [symbol] |
| | B | | | | monoamide | | acid | | VGHLM-N [symbol] |
| | over 12 | | | | | | | | (SEQ ID N [symbol] |
| | minutes | | | | | | | | 4) |
| L217 | 10-46% | 4.38 | 2093.37 | NT** | DO3A- | Gly | 4-aminobenzoic | none | QKYGNQ [symbol] |
| | B | | | | monoamide | | acid | | VGHLM-N [symbol] |
| | over 12 | | | | | | | | (SEQ ID N [symbol] |
| | minutes | | | | | | | | 5) |
| L218 | 10-46% | 6.13 | 2154.45 | NT** | DO3A- | Gly | 4-aminobenzoic | none | LGNQWA [symbol] |
| | B | | | | monoamide | | acid | | HLM-NH₂ |
| | over 12 | | | | | | | | (SEQ ID N [symbol] |
| | minutes | | | | | | | | 18) |
| L219 | 10-46% | 8.61 | 1588.84 | NT** | DO3A- | Gly | 4-aminobenzoic | (D)- | QWAVGH [symbol] |
| | B | | | | monoamide | | acid | Phe | NH-Pentyl |
| | over 12 | | | | | | | | (SEQ ID N [symbol] |
| | minutes | | | | | | | | 6) |
| L220 | 10-46% | 5.96 | 1516.75 | NT** | DO3A- | Gly | 4-aminobenzoic | none | QWSVaHL [symbol] |
| | B | | | | monoamide | | acid | | NH₂ (SEQ I [symbol] |
| | over 12 | | | | | | | | NO: 7) |
| | minutes | | | | | | | | |
| L221 | 10-46% | 7.96 | 1631.87 | NT** | DO3A- | Gly | 4-aminobenzoic | (D)- | QWAVGH [symbol] - |
| | B | | | | monoamide | | acid | Phe | NH₂ (SEQ [symbol] |
| | over 12 | | | | | | | | NO: 8) |
| | minutes | | | | | | | | |
| L222 | 10-46% | 6.61 | 1695.91 | NT** | DO3A- | Gly | 4-aminobenzoic | (D)- | QWAV-BaL [symbol] |
| | B | | | | monoamide | | acid | Tyr | HFNle-NH₂ |
| | over 12 | | | | | | | | (SEQ ID N [symbol] |
| | minutes | | | | | | | | 9) |
| L223 | 10-46% | 7.48 | 1679.91 | NT** | DO3A- | Gly | 4-aminobenzoic | Phe | QWAV-BaL [symbol] |
| | B | | | | monoamide | | acid | | HFNle-NH₂ |
| | over 12 | | | | | | | | (SEQ ID N [symbol] |
| | minutes | | | | | | | | 9) |
| L224 | 10-46% | 5.40 | 1419.57 | NT** | DO3A- | Gly | 4-aminobenzoic | none | QWAGHFL |
| | B | | | | monoamide | | acid | | NH₂ (SEQ I [symbol] |
| | over 12 | | | | | | | | NO: 10) |
| | minutes | | | | | | | | |
| L225 | 10-46% | 8.27 | 1471.71 | NT** | DO3A- | Gly | 4-aminobenzoic | none | LWAVGSF [symbol] |
| | B | | | | monoamide | | acid | | NH₂ (SEQ I [symbol] |
| | over 12 | | | | | | | | NO: 11) |
| | minutes | | | | | | | | |
| L226 | 10-46% | 5.12 | 1523.75 | NT** | DO3A- | Gly | 4-aminobenzoic | none | HWAVGH [symbol] - |
| | B | | | | monoamide | | acid | | NH₂ (SEQ [symbol] |
| | over 12 | | | | | | | | NO: 12) |
| | minutes | | | | | | | | |
| L227 | 10-46% | 6.61 | 1523.75 | NT** | DO3A- | Gly | 4-aminobenzoic | none | LWATGHF [symbol] - |
| | B | | | | monoamide | | acid | | NH₂ (SEQ [symbol] |
| | over 12 | | | | | | | | NO: 16) |
| | minutes | | | | | | | | |
| L228 | 10-46% | 5.77 | 1511 | NT** | DO3A- | Gly | 4-aminobenzoic | none | QWAVGH [symbol] - |
| | B | | | | monoamide | | acid | | NH₂ (SEQ [symbol] |
| | over 12 | | | | | | | | NO: 13) |
| | minutes | | | | | | | | |
| L233 | 5-35% | 7.04 | 1502.71 | 4.8 | DO3A- | Gly | 3-aminobenzoic | none | BBN(7-14)* |
| | B over | | | | monoamide | | acid | | |
| | 10 min | | | | | | | | |
| L234 | 20-80% | 1.95 | 1552.76 | 3 | DO3A- | Gly | 6-aminonaphthoic | none | BBN(7-14)* |
| | over 10 | | | | monoamide | | acid | | |
| | minutes | | | | | | | | |
| L235 | 20-80% | 1.95 | 1515.72 | 7 | DO3A- | Gly | 4-methylamino- | none | BBN(7-14)* |
| | over 10 | | | | monoamide | | benzoic acid | | |
| | minutes | | | | | | | | |
| L237 | 20-80% | 1.52 | 1538.68 | 5 | Cm4pm10d | Gly | 4-aminobenzoic | none | BBN(7-14)* |
| | over 10 | | | | 2a | | acid | | |
| | minutes | | | | | | | | |
| L238 | 5-35% | 7.17 | 1462.70 | 1.5 | N,N- | Gly | 4-aminobenzoic | none | BBN(7-14)* |
| | B over | | | | dimethyl- | | acid | | |
| | 10 min | | | | glycine-Ser- | | | | |
| | | | | | Cys(Acm)-Gly | | | | |
| L239 | 20-80% | 3.36 | 1733.16 | 4.5 | N,N- | Gly | 3-amino-3- | none | BBN(7-14)* |
| | over 10 | | | | dimethyl- | | deoxycholic acid | | |
| | minutes | | | | glycine-Ser- | | | | |
| | | | | | Cys(Acm)-Gly | | | | |
| L240 | 20-80% | 1.55 | 1532.73 | 4 | DO3A- | Gly | 3-methoxy-4- | none | BBN(7-14)* |
| | over 10 | | | | monoamide | | aminobenzoic | | |
| | minutes | | | | | | acid | | |
| L241 | 20-80% | 1.63 | 1535.68 | 4 | DO3A- | Gly | 3-chloro-4- | none | BBN(7-14)* |
| | over 10 | | | | monoamide | | aminobenzoic | | |
| | minutes | | | | | | acid | | |
| L242 | 20-80% | 1.55 | 1516.75 | 5 | DO3A- | Gly | 3-methyl-4- | none | BBN(7-14)* |
| | over 10 | | | | monoamide | | aminobenzoic | | |
| | minutes | | | | | | acid | | |
| L243 | 20-80% | 1.57 | 1518.70 | 14 | DO3A- | Gly | 3-hydroxy-4- | none | BBN(7-14)* |
| | over 10 | | | | monoamide | | aminobenzoic | | |
| | minutes | | | | | | acid | | |
| L244 | 5-50% | 4.61 | 1898.16 | >50 | (DO3A- | N,N′-Bis(2- | none | none | BBN(7-14)* |
| | over 10 | | | | monoamide)₂ | aminoethyl)- | | | |
| | minutes | | | | | succinamic | | | |
| | | | | | | acid | | | |
| L300 | 10-46% | — | — | — | DO3A- | Gly | 4-aminobenzoic | none | QWAVGH [symbol] |
| | over 10 | | | | monoamide | | acid | | NH₂ (SEQ I [symbol] |
| | minutes | | | | | | | | NO: 22) |
| L301 | 20-45% | 7.18 | — | — | DO3A- | none | 4-aminomethyl- | L-1- | BBN(7-14)* |
| | over 15 | | | | monoamide | | benzoic acid | Naph- | |
| | minutes | | | | | | | thyl- | |
| | | | | | | | | alanine | |
| L302 | — | — | — | — | DO3A- | Gly | 4-aminobenzoic | none | QWAVGN [symbol] |
| | | | | | monoamide | | acid | | H-L-M-NH₂ |
| | | | | | | | | | (SEQ ID N [symbol] |
| | | | | | | | | | 15) |
| L500 | — | — | 1515.7 | — | Aazta | Gly | 4-aminobenzoic | none | BBN(7-14)* |
| | | | | | | | acid | | |
| L501 | — | — | 1569.7 | — | CyAazta | Gly | 4-aminobenzoic | none | BBN(7-14)* |
| | | | | | | | acid | | |

*BBN(7-14) is the sequence QWAVGHLM (SEQ ID NO: 1)
**NT is defined as “not tested.”
***BOA is defined as (1R)-1-(Bis{2-[bis(carboxymethyl)amino]ethyl}amino)propane-1,3-dicarboxylic acid.
¹HPLC method refers to gradient change that occurs over the 10 minute time for the HPLC gradient.
²HPLC RT refers to the retention time of the compound in the HPLC.
³MS refers to mass spectra where molecular weight is calculated from mass/unit charge (m/e).
⁴IC₅₀ refers to the concentration of compound to inhibit 50% binding of iodinated bombesin to a GRP receptor on cells. [symbol]
indicates data missing or illegible when filed

A subset of compounds containing preferred linkers and various GRP receptor targeting peptides are set forth in Table 4. These compounds may be prepared using the methods disclosed herein, particularly in the Examples, as well as by similar methods known to one skilled in the art.

TABLE 4
Compounds Containing Linkers of the Invention With Various GRP-R Targeting Moieties

| | HPLC | HPLC | | | | | | | |
|---|---|---|---|---|---|---|---|---|---|
| Compound | method¹ | RT² | MS³ | IC50⁵ | M | N | O | P | G |
| L214 | 10-46% B over | 7.36 | 1649.91 | NT** | DO3A- | Gly | 4-aminobenzoic | (D)-Phe | BBN(7-14)* |
| | 12 minutes | | | | monoamide | | acid | | |
| L215 | 10-46% B over | 5.08 | 2071.37 | NT** | DO3A- | Gly | 4-aminobenzoic | none | QRLGNQ |
| | 12 minutes | | | | monoamide | | acid | | WAVGH [symbol] |
| | | | | | | | | | M-NH₂ |
| | | | | | | | | | (SEQ ID |
| | | | | | | | | | NO: 3) |
| L216 | 10-46% B over | 4.94 | 2121.38 | NT** | DO3A- | Gly | 4-aminobenzoic | none | QRYGNQ |
| | 12 minutes | | | | monoamide | | acid | | WAVGH [symbol] |
| | | | | | | | | | M-NH₂ |
| | | | | | | | | | (SEQ ID |
| | | | | | | | | | NO: 4) |
| L217 | 10-46% B over | 4.38 | 2093.37 | NT** | DO3A- | Gly | 4-aminobenzoic | none | QKYGNQ |
| | 12 minutes | | | | monoamide | | acid | | WAVGH [symbol] |
| | | | | | | | | | M-NH |
| | | | | | | | | | (SEQ ID |
| | | | | | | | | | NO: 5) |
| L218 | 10-46% B over | 6.13 | 2154.45 | NT** | DO3A- | Gly | 4-aminobenzoic | none | LGNQWA |
| | 12 minutes | | | | monoamide | | acid | | VGHLM- |
| | | | | | | | | | NH₂ (SEQ |
| | | | | | | | | | ID NO: 18 |
| L219 | 10-46% B over | 8.61 | 1588.84 | NT** | DO3A- | Gly | 4-aminobenzoic | (D)-Phe | QWAVG [symbol] |
| | 12 minutes | | | | monoamide | | acid | | L-NH- |
| | | | | | | | | | Pentyl |
| | | | | | | | | | (SEQ ID |
| | | | | | | | | | NO: 6) |
| L220 | 10-46% B over | 5.96 | 1516.75 | NT** | DO3A- | Gly | 4-aminobenzoic | none | QWAVaH |
| | 12 minutes | | | | monoamide | | acid | | M-NH₂ |
| | | | | | | | | | (SEQ ID |
| | | | | | | | | | NO: 7) |
| L221 | 10-46% B over | 7.96 | 1631.87 | NT** | DO3A- | Gly | 4-aminobenzoic | (D)-Phe | QWAVG [symbol] |
| | 12 minutes | | | | monoamide | | acid | | LL-NH₂ |
| | | | | | | | | | (SEQ ID |
| | | | | | | | | | NO: 8) |
| L222 | 10-46% B over | 6.61 | 1695.91 | NT** | DO3A- | Gly | 4-aminobenzoic | (D)-Tyr | QWAV- |
| | 12 minutes | | | | monoamide | | acid | | Bala-HF- |
| | | | | | | | | | Nle-NH₂ |
| | | | | | | | | | (SEQ ID |
| | | | | | | | | | NO: 9) |
| L223 | 10-46% B over | 7.48 | 1679.91 | NT** | DO3A- | Gly | 4-aminobenzoic | Phe | QWAV- |
| | 12 minutes | | | | monoamide | | acid | | Bala-HF- |
| | | | | | | | | | Nle-NH₂ |
| | | | | | | | | | (SEQ ID |
| | | | | | | | | | NO: 9) |
| L224 | 10-46% B over | 5.40 | 1419.57 | NT** | DO3A- | Gly | 4-aminobenzoic | none | QWAGH [symbol] - |
| | 12 minutes | | | | monoamide | | acid | | NH₂ (SE [symbol] |
| | | | | | | | | | ID NO: 10 [symbol] |
| L225 | 10-46% B over | 8.27 | 1471.71 | NT** | DO3A- | Gly | 4-aminobenzoic | none | LWAVGS |
| | 12 minutes | | | | monoamide | | acid | | M-NH₂ |
| | | | | | | | | | (SEQ ID |
| | | | | | | | | | NO: 11) |
| L226 | 10-46% B over | 5.12 | 1523.75 | NT** | DO3A- | Gly | 4-aminobenzoic | none | HWAVG [symbol] |
| | 12 minutes | | | | monoamide | | acid | | LM-NH₂ |
| | | | | | | | | | (SEQ ID |
| | | | | | | | | | NO: 12) |
| L227 | 10-46% B over | 6.61 | 1523.75 | NT** | DO3A- | Gly | 4-aminobenzoic | none | LWATGH |
| | 12 minutes | | | | monoamide | | acid | | M-NH2 |
| | | | | | | | | | (SEQ ID |
| | | | | | | | | | NO: 16) |
| L228 | 10-46% B over | 5.77 | 1511 | NT** | DO3A- | Gly | 4-aminobenzoic | none | QWAVG [symbol] |
| | 12 minutes | | | | monoamide | | acid | | FM-NH₂ |
| | | | | | | | | | (SEQ ID |
| | | | | | | | | | NO: 13) |
| L280 | — | — | — | — | DO3A- | Gly | (3β,5β 7a,12a)- | none | QWAVaH |
| | | | | | monoamide | | 3-amino-7,12- | | M-NH₂ |
| | | | | | | | dihydroxycholan- | | (SEQ ID |
| | | | | | | | 24-oic acid | | NO: 14) |
| L281 | — | — | — | — | DO3A- | Gly | (3β,5β 7a,12a)- | f | QWAVG [symbol] |
| | | | | | monoamide | | 3-amino-7,12- | | LM-NH₂* |
| | | | | | | | dihydroxycholan- | | |
| | | | | | | | 24-oic acid | | |
| L282 | — | — | — | — | DO3A- | Gly | (3β,5β 7a,12a)- | f | QWAVG [symbol] |
| | | | | | monoamide | | 3-amino-7,12- | | LL-NH₂ |
| | | | | | | | dihydroxycholan- | | (SEQ ID |
| | | | | | | | 24-oic acid | | NO: 8) |
| L283 | — | — | — | — | DO3A- | Gly | (3β,5β 7a,12a)- | f | QWAVG [symbol] |
| | | | | | monoamide | | 3-amino-7,12- | | LNH-pent [symbol] |
| | | | | | | | dihydroxycholan- | | (SEQ ID |
| | | | | | | | 24-oic acid | | NO: 6) |
| L284 | — | — | — | — | DO3A- | Gly | (3β,5β 7a,12a)- | y | QWAVBa |
| | | | | | monoamide | | 3-amino-7,12- | | HF-Nle- |
| | | | | | | | dihydroxycholan- | | NH₂ (SEQ |
| | | | | | | | 24-oic acid | | ID NO: 9) |
| L285 | — | — | — | — | DO3A- | Gly | (3β,5β 7a,12a)- | f | QWAVBa- |
| | | | | | monoamide | | 3-amino-7,12- | | HF-Nle- |
| | | | | | | | dihydroxycholan- | | NH₂ (SEQ |
| | | | | | | | 24-oic acid | | ID NO: 9) |
| L286 | — | — | — | — | DO3A- | Gly | (3β,5β 7a,12a)- | none | QWAVG [symbol] |
| | | | | | monoamide | | 3-amino-7,12- | | FL-NH₂ |
| | | | | | | | dihydroxycholan- | | (SEQ ID |
| | | | | | | | 24-oic acid | | NO: 22) |
| L287 | — | — | — | — | DO3A- | Gly | (3β,5β 7a,12a)- | none | QWAVG [symbol] |
| | | | | | monoamide | | 3-amino-7,12- | | MeHis-L- |
| | | | | | | | dihydroxycholan- | | M-NH₂ |
| | | | | | | | 24-oic acid | | (SEQ ID |
| | | | | | | | | | NO: 15) |
| L288 | — | — | — | — | DO3A- | Gly | (3β,5β 7a,12a)- | none | LWAVGS |
| | | | | | monoamide | | 3-amino-7,12- | | M-NH₂ |
| | | | | | | | dihydroxycholan- | | (SEQ ID |
| | | | | | | | 24-oic acid | | NO: 11) |
| L289 | — | — | — | — | DO3A- | Gly | (3β,5β 7a,12a)- | none | HWAVG [symbol] |
| | | | | | monoamide | | 3-amino-7,12- | | LM-NH₂ |
| | | | | | | | dihydroxycholan- | | (SEQ ID |
| | | | | | | | 24-oic acid | | NO: 12) |
| L290 | — | — | — | — | DO3A- | Gly | (3β,5β 7a,12a)- | none | LWATGH |
| | | | | | monoamide | | 3-amino-7,12- | | M-NH₂ |
| | | | | | | | dihydroxycholan- | | (SEQ ID |
| | | | | | | | 24-oic acid | | NO: 16) |
| L291 | — | — | — | — | DO3A- | Gly | (3β,5β 7a,12a)- | none | QWAVG [symbol] |
| | | | | | monoamide | | 3-amino-7,12- | | FM-NH₂ |
| | | | | | | | dihydroxycholan- | | (SEQ ID |
| | | | | | | | 24-oic acid | | NO: 13) |
| L292 | — | — | — | — | DO3A- | Gly | 3β,5β 7α,12α)- | QRLGN | QWAVG [symbol] |
| | | | | | monoamide | | 3-amino-7,12- | | LM-NH₂* |
| | | | | | | | dihydroxycholan- | | |
| | | | | | | | 24-oic acid | | |
| L293 | — | — | — | — | DO3A- | Gly | 3β,5β 7α,12α)- | QRYGN | QWAVG [symbol] |
| | | | | | monoamide | | 3-amino-7,12- | | LM-NH₂* |
| | | | | | | | dihydroxycholan- | | |
| | | | | | | | 24-oic acid | | |
| L294 | — | — | — | — | DO3A- | Gly | 3β,5β 7α,12α)- | QKYGN | QWAVG [symbol] |
| | | | | | monoamide | | 3-amino-7,12- | | LM-NH₂* |
| | | | | | | | dihydroxycholan- | | |
| | | | | | | | 24-oic acid | | |
| L295 | — | — | — | — | Pglu-Q-Lys | Gly | 3β,5β 7α,12α)- | LG-N | QWAVG [symbol] |
| | | | | | (DO3A- | | 3-amino-7,12- | | LM-NH₂* |
| | | | | | monoamide) | | dihydroxycholan- | | |
| | | | | | | | 24-oic acid | | |
| L304 | — | — | — | — | DO3A- | Gly | 3-amino-3- | none | QRYGNQ |
| | | | | | monoamide | | deoxycholic | | WAVGH [symbol] |
| | | | | | | | acid | | M-NH₂ |
| | | | | | | | | | (SEQ ID |
| | | | | | | | | | NO: 4) |
| L305 | — | — | — | — | DO3A- | Gly | 3-amino-3- | none | QKYGNQ |
| | | | | | monoamide | | deoxycholic | | WAVGH [symbol] |
| | | | | | | | acid | | M-NH₂ |
| | | | | | | | | | (SEQ ID |
| | | | | | | | | | NO: 5) |
| L306 | | | | | DO3A- | Gly | 3-amino-3- | none | LGNQWA |
| | | | | | monoamide | | deoxycholic | | VGHLM- |
| | | | | | | | acid | | NH₂ (SEQ |
| | | | | | | | | | ID NO: 18 |

*BBN(7-14) is the sequence QWAVGHLM (SEQ ID NO: 1) [symbol]
indicates data missing or illegible when filed

### 2D. Other Linking Groups

Other linking groups which may be used within the linker N—O—P include a chemical group that serves to couple the GRP receptor targeting peptide to the metal chelator or optical label while not adversely affecting either the targeting function of the GRP receptor targeting peptide or the metal complexing function of the metal chelator or the detectability of the optical label. Suitable other linking groups include peptides (i.e., amino acids linked together) alone, a non-peptide group (e.g., hydrocarbon chain) or a combination of an amino acid sequence and a non-peptide spacer.

In one embodiment, other linking groups for use within the linker N—O—P include L-glutamine and hydrocarbon chains, or a combination thereof.

In another embodiment, other linking groups for use within the linker N—O—P include a pure peptide linking group consisting of a series of amino acids (e.g., diglycine, triglycine, gly-gly-glu, gly-ser-gly, etc.), in which the total number of atoms between the N-terminal residue of the GRP receptor targeting peptide and the metal chelator or the optical label in the polymeric chain is ≦12 atoms.

In yet a further embodiment, other linking groups for use within the linker N—O—P can also include a hydrocarbon chain [i.e., R₁—(CH₂)ₙ—R₂] wherein n is 0-10, preferably n=3 to 9, R₁ is a group (e.g., H₂N—, HS—, —COOH) that can be used as a site for covalently linking the ligand backbone or the preformed metal chelator or metal complexing backbone or optical label; and R₂ is a group that is used for covalent coupling to the N-terminal NH₂-group of the GRP receptor targeting peptide (e.g., R₂ is an activated COOH group). Several chemical methods for conjugating ligands (i.e., chelators) or preferred metal chelates to biomolecules have been well described in the literature [Wilbur, 1992; Parker, 1990; Hermanson, 1996; Frizberg et al., 1995]. One or more of these methods could be used to link either the uncomplexed ligand (chelator) or the radiometal chelate or optical label to the linker or to link the linker to the GRP receptor targeting peptides. These methods include the formation of acid anhydrides, aldehydes, arylisothiocyanates, activated esters, or N-hydroxysuccinimides [Wilbur, 1992; Parker, 1990; Hermanson, 1996; Frizberg et al., 1995].

In a preferred embodiment, other linking groups for use within the linker N—O—P may be formed from linker precursors having electrophiles or nucleophiles as set forth below:
LP1: a linker precursor having on at least two locations of the linker the same electrophile E1 or the same nucleophile Nu1;
LP2: a linker precursor having an electrophile E1 and on another location of the linker a different electrophile E2;
LP3: a linker precursor having a nucleophile Nu1 and on another location of the linker a different nucleophile Nu2; or
LP4: a linker precursor having one end functionalized with an electrophile E1 and the other with a nucleophile Nu1.

The preferred nucleophiles Nu1/Nu2 include-OH, —NH, —NR, —SH, —HN—NH₂, —RN—NH₂, and —RN—NHR′, in which R′ and R are independently selected from the definitions for R given above, but for R′ is not H.

The preferred electrophiles E1/E2 include —COOH, —CH═O (aldehyde), —CR═OR′ (ketone), —RN—C═S, —RN—C═O, —S—SO₂-pyridyl, —SO₂—Y, —CH₂C(═O)Y, and

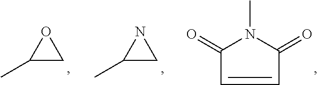

wherein Y can be selected from the following groups:

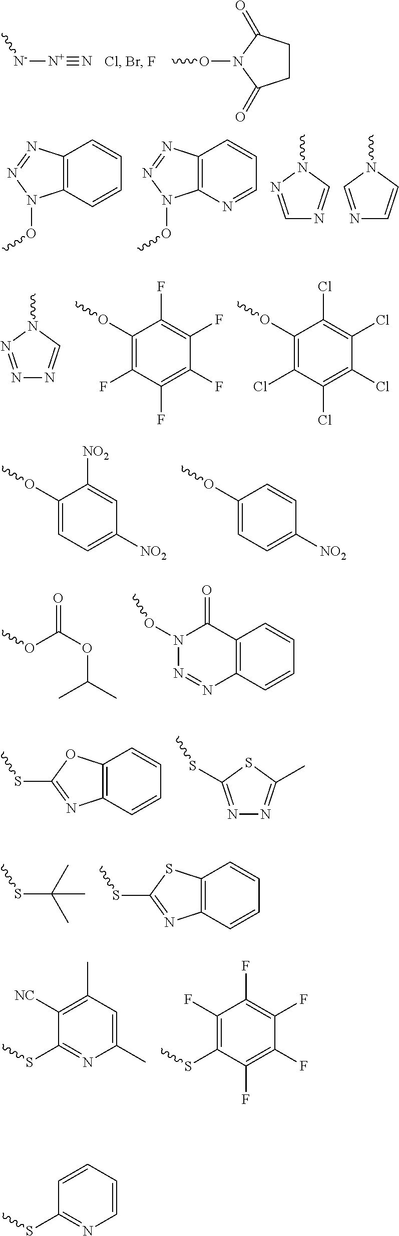

### 3. GRP Receptor Targeting Peptide

The GRP receptor targeting peptide (i.e., G in the formula M-N—O—P-G) is any peptide, equivalent, derivative or analogue thereof which has a binding affinity for the GRP receptor family.

The GRP receptor targeting peptide may take the form of an agonist or an antagonist. A GRP receptor targeting peptide agonist is known to “activate” the cell following binding with high affinity and may be internalized by the cell. Conversely, GRP receptor targeting peptide antagonists are known to bind only to the GRP receptor on the cell without being internalized by the cell and without “activating” the cell. In a preferred embodiment, the GRP receptor targeting peptide is an agonist.

In a more preferred embodiment of the present invention, the GRP agonist is a bombesin (BBN) analogue and/or a derivative thereof. The BBN derivative or analog thereof preferably contains either the same primary structure of the BBN binding region (i.e., BBN(7-14) (SEQ ID NO: 1) or similar primary structures, with specific amino acid substitutions that will specifically bind to GRP receptors with better or similar binding affinities as BBN alone (i.e., Kd<25 nM). Suitable compounds include peptides, peptidomimetics and analogues and derivatives thereof. The presence of L-methionine (Met) at position BBN-14 will generally confer agonistic properties while the absence of this residue at BBN-14 generally confers antagonistic properties [Hoffken, 1994]. Some useful bombesin analogues are disclosed in U.S. Patent Pub. 2003/0224998, incorporated here in its entirety.

It is well documented in the art that there are a few and selective number of specific amino acid substitutions in the BBN (8-14) binding region (e.g., D-Ala¹¹ for L-Gly¹¹ or D-Trp⁸ for L-Trp), which can be made without decreasing binding affinity [Leban et al., 1994; Qin et al., 1994; Jensen et al., 1993]. In addition, attachment of some amino acid chains or other groups to the N-terminal amine group at position BBN-8 (i.e., the Trp⁸ residue) can dramatically decrease the binding affinity of BBN analogues to GRP receptors [Davis et al., 1992; Hoffken, 1994; Moody et al., 1996; Coy, et al., 1988; Cai et al., 1994]. In a few cases, it is possible to append additional amino acids or chemical moieties without decreasing binding affinity.

Analogues of BBN receptor targeting peptides include molecules that target the GRP receptors with avidity that is greater than or equal to BBN, as well as muteins, retropeptides and retro-inverso-peptides of GRP or BBN. One of ordinary skill will appreciate that these analogues may also contain modifications which include substitutions, and/or deletions and/or additions of one or several amino acids, insofar that these modifications do not negatively alter the biological activity of the peptides described therein. These substitutions may be carried out by replacing one or more amino acids by their synonymous amino acids. Synonymous amino acids within a group are defined as amino acids that have sufficient physicochemical properties to allow substitution between members of a group in order to preserve the biological function of the molecule.

Deletions or insertions of amino acids may also be introduced into the defined sequences provided they do not alter the biological functions of said sequences. Preferentially such insertions or deletions should be limited to 1, 2, 3, 4 or 5 amino acids and should not remove or physically disturb or displace amino acids which are critical to the functional conformation. Muteins of the GRP receptor targeting peptides described herein may have a sequence homologous to the sequence disclosed in the present specification in which amino acid substitutions, deletions, or insertions are present at one or more amino acid positions. Muteins may have a biological activity that is at least 40%, preferably at least 50%, more preferably 60-70%, most preferably 80-90% of the peptides described herein. However, they may also have a biological activity greater than the peptides specifically exemplified, and thus do not necessarily have to be identical to the biological function of the exemplified peptides. Analogues of GRP receptor targeting peptides also include peptidomimetics or pseudopeptides incorporating changes to the amide bonds of the peptide backbone, including thioamides, methylene amines, and E-olefins. Also peptides based on the structure of GRP, BBN or their peptide analogues with amino acids replaced by N-substituted hydrazine carbonyl compounds (also known as aza amino acids) are included in the term analogues as used herein.

The GRP receptor targeting peptide can be prepared by various methods depending upon the selected chelator. The peptide can generally be most conveniently prepared by techniques generally established and known in the art of peptide synthesis, such as the solid-phase peptide synthesis (SPPS) approach. Solid-phase peptide synthesis (SPPS) involves the stepwise addition of amino acid residues to a growing peptide chain that is linked to an insoluble support or matrix, such as polystyrene. The C-terminal residue of the peptide is first anchored to a commercially available support with its amino group protected with an N-protecting agent such as a t-butyloxycarbonyl group (Boc) or a fluorenylmethoxycarbonyl (Fmoc) group. The amino protecting group is removed with suitable deprotecting agents such as TFA in the case of Boc or piperidine for Fmoc and the next amino acid residue (in N-protected form) is added with a coupling agent such as N,N′-dicyclohexylcarbodiimide (DCC), or N,N′-diisopropylcarbodiimide (DIC) or 2-(1H-benzotriazol-1-yl)-1,1,3,3-tetramethyluronium hexafluorophosphate (HBTU). Upon formation of a peptide bond, the reagents are washed from the support. After addition of the final residue, the peptide is cleaved from the support with a suitable reagent such as trifluoroacetic acid (TFA) or hydrogen fluoride (HF).

The linker may then be coupled to form a conjugate by reacting the free amino group of the Trp⁸ residue of the GRP receptor targeting peptide with an appropriate functional group of the linker. The entire construct of chelator, linker and targeting moiety discussed above may also be assembled on resin and then cleaved by agency of suitable reagents such as trifluoroacetic acid or HF, as well.

Bombesin (7-14) is subject to proteolytic cleavage in vitro and in vivo, which shortens the half-life of the peptide. It is well known in the literature that the amide bond of the backbone of the polypeptide may be substituted and retain activity, while resisting proteolytic cleavage. For example, to reduce or eliminate undesired proteolysis, or other degradation pathways that diminish serum stability, resulting in reduced or abolished bioactivity, or to restrict or increase conformational flexibility, it is common to substitute amide bonds within the backbone of the peptides with functionality that mimics the existing conformation or alters the conformation in the manner desired. Such modifications may produce increased binding affinity or improved pharmacokinetic behavior. It is understood that those knowledgeable in the art of peptide synthesis can make the following amide bond-changes for any amide bond connecting two amino acids (e.g., amide bonds in the targeting moiety, linker, chelator, etc.) with the expectation that the resulting peptides could have the same or improved activity: insertion of alpha-N-methylamides or backbone thioamides, removal of the carbonyl to produce the cognate secondary amines, replacement of one amino acid with an aza-aminoacid to produce semicarbazone derivatives, and use of E-olefins and substituted E-olefins as amide bond surrogates. The hydrolysis can also be prevented by incorporation of a D-amino acid of one of the amino acids of the labile amide bond, or by alpha-methyl aminoacid derivatives. Backbone amide bonds have also been replaced by heterocycles such as oxazoles, pyrrolidinones, imidazoles, as well as ketomethylenes and fluoroolefins.

Some specific compounds including such amide bond modifications are listed in Table 4a. The abbreviations used in Table 4a for the various amide bond modifications are exemplified below:

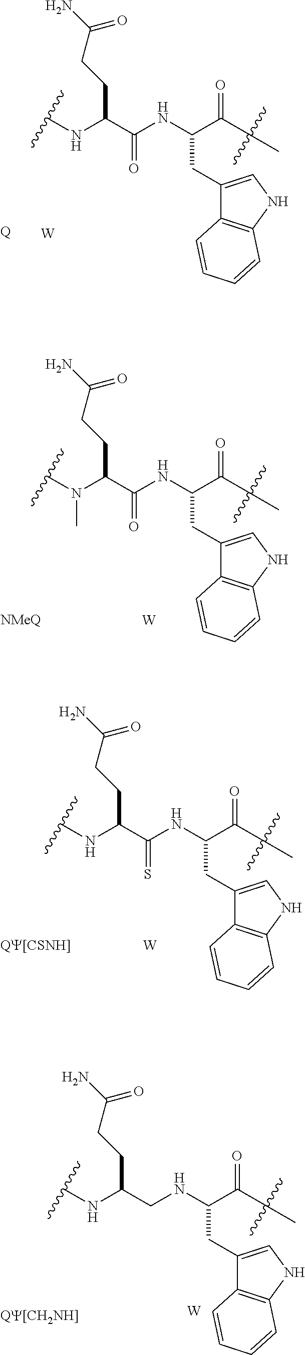

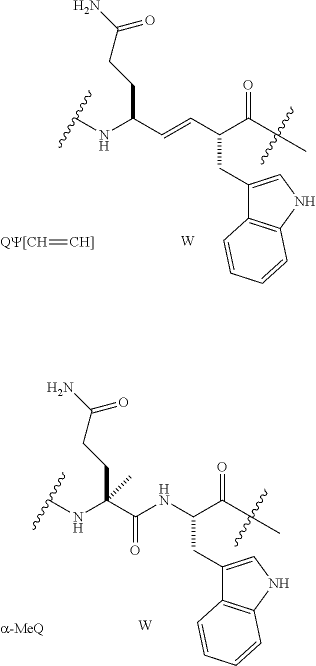

TABLE 4A
Preferred Amide Bond Modified Analogs

| Compound | M-N-O-P | BBN Analogue | | | | | | | |
|---|---|---|---|---|---|---|---|---|---|
| L401 | DO3A- | Nme-Q | W | A | V | G | H | L | M-NH₂ |
| | monoamide-G- | | | | | | | | |
| | Abz4 | | | | | | | | |
| L402 | DO3A- | Q-Ψ | W | A | V | G | H | L | M-NH₂ |
| | monoamide-G- | [CSNH] | | | | | | | |
| | Abz4 | | | | | | | | |
| L403 | DO3A- | Q-Ψ | W | A | V | G | H | L | M-NH₂ |
| | monoamide-G- | [CH₂NH] | | | | | | | |
| | Abz4 | | | | | | | | |
| L404 | DO3A- | Q-Ψ | W | A | V | G | H | L | M-NH₂ |
| | monoamide-G- | [CH═CH] | | | | | | | |
| | Abz4 | | | | | | | | |
| L405 | DO3A- | α-MeQ | W | A | V | G | H | L | M-NH₂ |
| | monoamide-G- | | | | | | | | |
| | Abz4 | | | | | | | | |
| L406 | DO3A- | Q | Nme-W | A | V | G | H | L | M-NH₂ |
| | monoamide-G- | | | | | | | | |
| | Abz4 | | | | | | | | |
| L407 | DO3A- | Q | W-Ψ | A | V | G | H | L | M-NH₂ |
| | monoamide-G- | | [CSNH] | | | | | | |
| | Abz4 | | | | | | | | |
| L408 | DO3A- | Q | W-Ψ | A | V | G | H | L | M-NH₂ |
| | monoamide-G- | | [CH₂NH] | | | | | | |
| | Abz4 | | | | | | | | |
| L409 | DO3A- | Q | W-Ψ | A | V | G | H | L | M-NH₂ |
| | monoamide-G- | | [CH═CH] | | | | | | |
| | Abz4 | | | | | | | | |
| L410 | DO3A- | Q | α-MeW | A | V | G | H | L | M-NH₂ |
| | monoamide-G- | | | | | | | | |
| | Abz4 | | | | | | | | |
| L411 | DO3A- | Q | W | Nme-A | V | G | H | L | M-NH₂ |
| | monoamide-G- | | | | | | | | |
| | Abz4 | | | | | | | | |
| L412 | DO3A- | Q | W | A-Ψ | V | G | H | L | M-NH₂ |
| | monoamide-G- | | | [CSNH] | | | | | |
| | Abz4 | | | | | | | | |
| L413 | DO3A- | Q | W | A-Ψ | V | G | H | L | M-NH₂ |
| | monoamide-G- | | | [CH₂NH] | | | | | |
| | Abz4 | | | | | | | | |
| L414 | DO3A- | Q | W | Aib | V | G | H | L | M-NH₂ |
| | monoamide-G- | | | | | | | | |
| | Abz4 | | | | | | | | |
| L415 | DO3A- | Q | W | A | V | Sar | H | L | M-NH₂ |
| | monoamide-G- | | | | | | | | |
| | Abz4 | | | | | | | | |
| L416 | DO3A- | Q | W | A | V | G-Ψ | H | L | M-NH₂ |
| | monoamide-G- | | | | | [CSNH] | | | |
| | Abz4 | | | | | | | | |
| L417 | DO3A- | Q | W | A | V | G-Ψ | H | L | M-NH₂ |
| | monoamide-G- | | | | | [CH═CH] | | | |
| | Abz4 | | | | | | | | |
| L418 | DO3A- | Q | W | A | V | Dala | H | L | M-NH₂ |
| | monoamide-G- | | | | | | | | |
| | Abz4 | | | | | | | | |
| L419 | DO3A- | Q | W | A | V | G | Nme- | L | M-NH₂ |
| | monoamide-G- | | | | | | His | | |
| | Abz4 | | | | | | | | |
| L420 | DO3A- | Q | W | A | V | G | H-Ψ | L | M-NH₂ |
| | monoamide-G- | | | | | | [CSNH] | | |
| | Abz4 | | | | | | | | |
| L421 | DO3A- | Q | W | A | V | G | H-Ψ | L | M-NH₂ |
| | monoamide-G- | | | | | | [CH₂NH] | | |
| | Abz4 | | | | | | | | |
| L422 | DO3A- | Q | W | A | V | G | H-Ψ | L | M-NH₂ |
| | monoamide-G- | | | | | | [CH═CH] | | |
| | Abz4 | | | | | | | | |
| L423 | DO3A- | Q | W | A | V | G | α-MeH | L | M-NH₂ |
| | monoamide-G- | | | | | | | | |
| | Abz4 | | | | | | | | |
| L424 | DO3A- | Q | W | A | V | G | H | Nme-L | M-NH₂ |
| | monoamide-G- | | | | | | | | |
| | Abz4 | | | | | | | | |
| L425 | DO3A- | Q | W | A | V | G | H | α-MeL | M-NH₂ |
| | monoamide-G- | | | | | | | | |
| | Abz4 | | | | | | | | |
| L300 | DO3A- | Q | W | A | V | G | H | F-L | NH₂ |
| | monoamide-G- | | | | | | | | |
| | ABz4 | | | | | | | | |

In the above table,QWAVGHLM-NH₂ is SEQ ID NO: 1 and QWAVGHFL-NH₂ (L300) is SEQ ID NO: 22.

### 4. Labeling and Administration of Radiopharmaceutical Compounds

Incorporation of the metal within the radiopharmaceutical conjugates can be achieved by various methods commonly known in the art of coordination chemistry. When the metal is ^{99m}Tc, a preferred radionuclide for diagnostic imaging, the following general procedure can be used to form a technetium complex. A peptide-chelator conjugate solution is formed by initially dissolving the conjugate in water, dilute acid, or in an aqueous solution of an alcohol such as ethanol. The solution is then optionally degassed to remove dissolved oxygen. When an —SH group is present in the peptide, a thiol protecting group such as Acm (acetamidomethyl), trityl or other thiol protecting group may optionally be used to protect the thiol from oxidation. The thiol protecting group(s) are removed with a suitable reagent, for example with sodium hydroxide, and are then neutralized with an organic acid such as acetic acid (pH 6.0-6.5). Alternatively, the thiol protecting group can be removed in situ during technetium chelation. In the labeling step, sodium pertechnetate obtained from a molybdenum generator is added to a solution of the conjugate with a sufficient amount of a reducing agent, such as stannous chloride, to reduce technetium and is either allowed to stand at room temperature or is heated. The labeled conjugate can optionally be separated from the contaminants ^{99m}TcO₄⁻ and colloidal ^{99m}TcO₂ chromatographically, for example with a C-18 Sep Pak cartridge [Millipore Corporation, Waters Chromatography Division, 34 Maple Street, Milford, Mass. 01757] or by HPLC using methods known to those skilled in the art.

In an alternative method, the labeling can be accomplished by a transchelation reaction. In this method, the technetium source is a solution of technetium that is reduced and complexed with labile ligands prior to reaction with the selected chelator, thus facilitating ligand exchange with the selected chelator. Examples of suitable ligands for transchelation includes tartrate, citrate, gluconate, and heptagluconate. It will be appreciated that the conjugate can be labeled using the techniques described above, or alternatively, the chelator itself may be labeled and subsequently coupled to the peptide to form the conjugate; a process referred to as the “prelabeled chelate” method. Re and Tc are both in row VIIB of the Periodic Table and they are chemical congeners. Thus, for the most part, the complexation chemistry of these two metals with ligand frameworks that exhibit high in vitro and in vivo stabilities are the same [Eckelman, 1995] and similar chelators and procedures can be used to label with Re. Many ^{99m}Tc or 186/188Re complexes, which are employed to form stable radiometal complexes with peptides and proteins, chelate these metals in their +5 oxidation state [Lister-James et al., 1997]. This oxidation state makes it possible to selectively place ^{99m}Tc- or ^{186/188}Re into ligand frameworks already conjugated to the biomolecule, constructed from a variety of ^{99m}Tc(V) and/or ^{186/188}Re(V) weak chelates (e.g., ^{99m}Tc-glucoheptonate, citrate, gluconate, etc.) [Eckelman, 1995; Lister-James et al., 1997; Pollak et al., 1996]. These references are hereby incorporated by reference in their entirety.

The positron-emitting radioisotope ⁶⁸Ga is a preferred radiometal for PET (Positron Emission Tomography) imaging. An important characteristic of ⁶⁸Ga is its cyclotron-independent availability via the ⁶⁸Ge/⁶⁸Ga radionuclide generator system. The relatively long-lived parent 68Ge (half-life [t_{1/2}]=270.95 d) produces a short-lived ⁶⁸Ga (t_{1/2}=67.71 min), which subsequently decays to stable 68Zn. 68Ga is an excellent positron emitter, with 89% positron branching accompanied by low photon emission (1,077 keV, 3.22%). ⁶⁸Ge/⁶⁸Ga radionuclide generators have been under development and investigation for almost 50 years. For a recent review, see Rosch et al. (Rosch F, Knapp F F. Radionuclide generators. In: Vértes A, Nagy S, Klencsar Z, Rösch F, eds. Handbook of Nuclear Chemistry. Dordrecht, The Netherlands: Kluwer Academic Publishers; 2003; 4:81-118.)

Today, the most common commercially available ⁶⁸Ge/⁶⁸Ga radionuclide generator is based on a TiO₂ solid phase but other solid phases can be used. “Ionic” ⁶⁸Ga³⁺ is eluted from the generator using, for example, a 0.1N HCl solution, although other eluants may also be used. The ⁶⁸Ga yield is >60% in 5 mL of the eluate; the breakthrough of the long-lived parent ⁶⁸Ge usually does not exceed 5.10 ³%. The eluant may either be used directly for labeling, or it may be prepurified to concentrate it and/or to remove ⁶⁸Ge and other trace metals prior to labeling. For example, Hofmann et al (Hofmann M, Maecke H R, Borner A R, et al. Biokinetics and imaging with the somatostatin receptor PET radioligand ⁶⁸Ga-DOTATOC: preliminary data. Eur J Nucl Med. 2001; 28:1751-1757) and Meyer et al (Meyer G-J, Macke H R, Schuhmacher J, Knapp W H, Hofmann M. ⁶⁸Ga-Labelled DOTA-derivatised peptide ligands. Eur J Nucl Med. 2004; 31:1097-1104) have described concentration methods wherein the initial generator eluate is treated with strong acid (e.g. 9.5N HCl). Under these conditions, ⁶⁸Ga can be adsorbed on a strong anion-exchanger as anionic chloro complexes of ⁶⁸Ga(III). After a washing step with 5.5N HCl, the resin is flushed with a stream of nitrogen and then eluted with H₂O in small volumes. This strategy separates ⁶⁸Ge from ⁶⁸Ga.

Konstantin (K. P. Zhernosekov, D. V. Filosofov, R. P. Baum, P. Aschoff, H. Bihl, A. A. Razbash, M. Jahn, M. Jennewein and F. Rosch, Processing of Generator-Produced ⁶⁸Ga for Medical Application, J. Nucl. Med. Vol. 48, 1741-1748) have described an alternate method wherein reconcentration and purification of the initial generator eluate are performed using a small column containing organic cation-exchanger resin that is eluted using hydrochloric acid/acetone to remove ⁶⁸Ga. The purified fraction can be used for the labeling of chelator-containing peptides such as octreotide derivatives, either in pure aqueous solution or in buffers.

Another approach is to fractionate the initial generator eluate. Collecting only the portion of the eluent that contains the highest concentrations of ⁶⁸Ga helps to overcome problems such as eluate volume, acidic pH, and the presence of ⁶⁸Ge and chemical impurities (Breeman WAP, de Jong M, de Blois E, Bernard B F, Konijnenberg M, Krenning E P. Radiolabelling DOTA-peptides with ⁶⁸Ga. Eur JNucl Med. 2004; 32:478-485). Alternatively, the ⁶⁸Ga from generator eluant can be extracted for example, into an organic solvent such as methyl ethyl ketone, e.g. as described by Bokhari et al (Bokhari T H, Mushtaq A, Khan I U, Concentration of ⁶⁸Ga via solvent extraction, Appl Radiat Isot. 2009; 67(1):100-102). Evaporation of the solvent concentrates the radioisotope, which is then diluted in buffer for radiolabeling. In some cases, the eluent is not prepurified, but instead, ⁶⁸Ge breakthrough is removed after radiolabeling, using techniques such as solid phase extraction or HPLC.

Regardless of how the radioisotope solution is obtained or purified, radiolabeling is typically performed in an aqueous or aqueous/organic mixture at a suitable pH value for incorporation of the radiometal into the chelator. Macrocyclic chelators such as NOTA, DOTA and DO3A derivatives are typically used to bind the ⁶⁸Ga, although open-chain multidentate ligands such as N,N′-bis[2-hydroxy-5-(carboxyethyl)benzyl]ethylenediamine-N,N′-diacetic acid, the bis amine, bis-thiol ligands BAT-TECH (bis-aminoethanethiol-tetraethyl-cyclohexyl) and ethylenedicysteine (EC), N3S can also be used due to their rapid rate of labeling, which is important with short-lived isotopes such as ⁶⁸Ga. These chelators can be labeled at pH values from ˜2 to ˜7, most commonly at pH values between 3-5. If the pH is too low, radiometal does not incorporate well. If the pH is too high, competing reactions of Ga³⁺ with water and OH⁻ take place, leading to the formation of insoluble Ga-hydroxide (oxo) containing compounds known as radiocolloid. The proper pH for radiometal incorporation is typically maintained using a physiologically acceptable buffer such as acetate, citrate, bicarbonate, HEPES and the like. High buffer concentrations may be used if the eluant is provided in strong acid.

Sufficient ligand is added to provide the desired ⁶⁸Ga complex in high yield. The amount of ligand needed is determined by the radioconcentration, pH, buffer composition, nature of the chelator, time since the generator was last eluted and the quantity of competing metals present in the labeling solution. Competing metals can include Zn²⁺ obtained from the decay of ⁶⁸Ga³⁺, and Fe(III), Al(III) and the like that are present in the solvents used to elute the generator or are eluted from the generator itself. Typically, a ratio of >2:1 complexing ligand/Ga must be used. To incorporate the radiometal into the ligand, the reaction mixture is allowed to stand at room temperature, or is heated at a temperature of about 37° C. to ˜100° C. (either thermally or using a microwave), depending on the nature of the reactants.

The specific activity of the final product is an important consideration. If the system under study has a low receptor concentration and the radiolabeled product is not going to be purified to remove excess ligand, it can be important to minimize the amount of ligand that is used in the reaction, as this can compete with the radiolabeled product, thus reducing the effective signal at the target. In addition, some ligands have physiological effects that make it preferable to remove excess ligand. If desirable, impurities can be removed and specific activity can be raised by purification of the radiolabeled product to remove excess unlabeled chelator and/or chelator labeled with other metals, using techniques such as solid phase extraction, ion-exchange and/or reversed phase high pressure liquid chromatography.

If required, the compounds can be stabilized to prevent radiolytic damage to the compound prior to injection. Radiation stabilizers are known to those skilled in the art, and may include, for example, para-aminobenzoic acid, ascorbic acid, gentistic acid and the like, as well as those disclosed in US 2007/0269375 and WO 05/009393, incorporated by reference herein in their entirety, including selenium containing derivatives such as selenomethionine, cysteine derivatives, or dithiocarbamates. They may also contain solubilizers and bacteriostats, such as, for example, benzyl alcohol. Chelating agents such as EDTA or DTPA may also be added to bind to any unreacted “free” radiometal that remains after reaction.

⁶⁷Gallium-labeled compounds can also be prepared, using labeling methods similar to those described above. ⁶⁷Ga radioisotope is typically supplied in dilute acid solution as a chloride or citrate salt. Sufficient ligand must be used in labeling solutions to offset the presence of Zn(II) and other competing trace metals in labeling solutions.

### 5. Ga-AMBA

Ga-AMBA (or ⁶⁷Ga-AMBA and/or ⁶⁸Ga-AMBA) refers to the ⁶⁷Ga or ⁶⁸Ga labeled analog of AMBA and is also referred to herein as Ga-L70 (or ⁶⁷G-L70 and ⁶⁸Ga-L70). It is a preferred imaging agent of the invention and can be used as described herein to monitor therapeutic response to drugs which crosstalk with the GRP-R.

Chelating agents that bind Lu³⁺, such as DO3A, included in Lu-AMBA, also bind Ga³⁺. ⁶⁸Ga is particularly preferred isotope, as ⁶⁸Ga is a useful generator-produced isotope for PET imaging, having a t^{1/2}=68 minutes. Both ⁶⁷Ga and ⁶⁸Ga-AMBA target GRP-R positive cancers. An exemplary structure of such an analog is presented below:

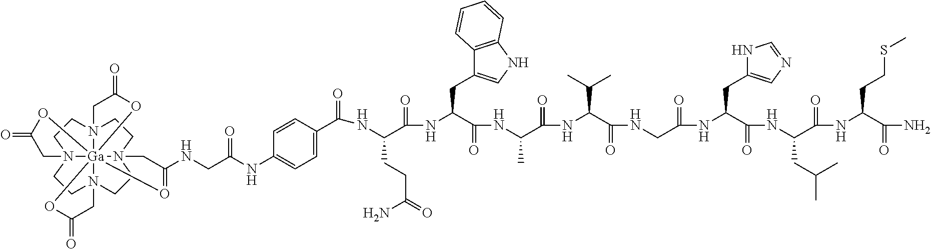

[DO3A Chelator][Linker][Targeting Group]

The molecule has three parts, the chelator, a linker group which controls receptor subtype specificity, and an octa-peptide targeting group (BBN 7-14) that is truncated from bombesin, the natural ligand for GRP-R. Lu-AMBA has a Kd ˜2-3 nM for GRP-R. Ga³⁺ and Lu³⁺ are both 3+ metal ions that bind similarly in multivalent aminocarboxylate ligands like the R-DO3A macrocycle used for Lu-AMBA (Lu-L70).

As shown in Table 5, it has been found that ⁶⁷Ga and ¹⁷⁷Lu-AMBA are biologically equivalent in a GRP-R positive PC-3 human prostate tumor-bearing mouse model. Similar results are expected with ⁶⁸Ga.

TABLE 5

| Organ/Tissue | ¹⁷⁷Lu-AMBA | ⁶⁷Ga-AMBA |
|---|---|---|
| In nude | Mass dose ~0.0026 μg | Mass dose = 0.0002 μg |
| mice implanted | n = 9 | n = 4 |
| with human tumor | 1 hour | 1 hour |
| PC-3 Tumor | 6.35 ± 2.23 | 3.90 ± 0.48 |
| (% ID/g) | | |
| Blood (% ID) | 0.24 ± 0.09 | 0.49 ± 0.11* |
| Liver (% ID) | 0.25 ± 0.08 | 0.35 ± 0.07 |
| Kidneys (% ID) | 2.95 ± 0.79 | 2.56 ± 0.48 |
| Pancreas (% ID) | 17.78 ± 4.07 | 16.60 ± 1.58 |
| GI (% ID) | 11.22 ± 3.29 | 12.17 ± 0.56 |
| Skin (% ID/g) | 0.33 ± 0.13 | 0.34 ± 0.07 |
| Muscle (% ID/g) | 0.09 ± 0.02 | 0.12 ± 0.08 |
| Blad/Urine (% ID) | 55.66 ± 7.28 | 52.16 ± 7.65 |

As shown in Table 5, the biodistribution of ¹⁷⁷Lu-AMBA was compared to the ⁶⁷Ga-AMBA (as a surrogate for ⁶⁸Ga-AMBA). The distribution to the PC-3 tumor target was not significantly different. Based on these data and the in vitro data, ¹⁷⁷Lu-AMBA is a reasonable surrogate to predict the behavior of Ga-AMBA in vitro and in vivo and is a preferred imaging agent of the invention. Ga-AMBA is also a preferred imaging agent to monitor therapeutic response to drugs which target receptors that cross talk with GRP-R.

### 6. Diagnostic and Therapeutic Uses

When labeled with diagnostically and/or therapeutically useful metals or optical labels, compounds of the present invention can be used to treat and/or detect any pathology involving overexpression of GRP receptors (or NMB receptors) by procedures established in the art of radiodiagnostics, radiotherapeutics and optical imaging. [See, e.g., Bushbaum, 1995; Fischman et al., 1993; Schubiger et al., 1996; Lowbertz et al., 1994; Krenning et al., 1994; examples of optical dyes include, but are not limited to those described in WO 98/18497, WO 98/18496, WO 98/18495, WO 98/18498, WO 98/53857, WO 96/17628, WO 97/18841, WO 96/23524, WO 98/47538, and references cited therein, hereby incorporated by reference in their entirety.]

GRP-R expression is highly upregulated in a variety of human tumors. See e.g., WO 99/62563. Thus, compounds of the invention may be widely useful in treating and diagnosing cancers, including prostate cancer (primary and metastatic), breast cancer (primary and metastatic), colon cancer, gastric cancer, pancreatic cancer, non small cell lung cancer, small cell lung cancer, gastrinomas, melanomas, glioblastomas, neuroblastomas, uterus leiomyosarcoma tumors, prostatic intraepithelial neoplasias [PIN], and ovarian cancer. Additionally, compounds of the invention may be useful to distinguish between conditions in which GRP receptors are upregulated and those in which they are not (e.g. chronic pancreatitis and ductal pancreatic carcinoma, respectively).

The compounds of the invention, which, as explained in more detail in the Examples, show greater specificity and higher uptake in tumors in vivo than compounds without the novel linkers disclosed herein, exhibit an improved ability to target GRP receptor-expressing tumors and thus to image or deliver radiotherapy to these tissues.

The diagnostic application of these compounds can be as a first line diagnostic screen for the presence of neoplastic cells using scintigraphic, optical, imaging, as an agent for targeting neoplastic tissue using hand-held radiation detection instrumentation in the field of radioimmuno guided surgery (RIGS), as a means to obtain dosimetry data prior to administration of the matched pair radiotherapeutic compound, as a means to assess GRP receptor activity as a function of treatment of GRP receptor targeted therapy over time, and as a means to assess GRP receptor activity as a function of non GRP receptor targeted therapies over time and thus indirectly assess the response of the targeted receptor to treatment.

The therapeutic application of these compounds can be defined as an agent that will be used as a first line therapy in the treatment of cancer, as combination therapy with a chemotherapeutic or other drug, and/or as a matched pair diagnostic/therapeutic agent. Treatment encompasses at least partial amelioration or alleviation of symptoms of a given condition. For example, treatment may result in a decrease in the size of a tumor or other diseased area, prevention of an increase in size of the tumor or diseased area, reduction in aberrant blood flow or otherwise normalizing the blood flow in the tumor, delaying time to progression of the tumor, increasing survival of the patient, etc. The matched pair concept refers to a single unmetallated compound which can serve as both a diagnostic and a therapeutic agent depending on the radiometal that has been selected for binding to the appropriate chelate. If the chelator cannot accommodate the desired metals, appropriate substitutions can be made to accommodate the different metals, while maintaining the pharmacology such that the behavior of the diagnostic compound in vivo can be used to predict the behavior of the radiotherapeutic compound. When utilized in conjunction with combination therapy, any suitable chemotherapeutic or drug may be used, including for example, antineoplastic agents, such as platinum compounds (e.g., spiroplatin, cisplatin, and carboplatin), methotrexate, adriamycin, mitomycin, ansamitocin, bleomycin, cytosine, arabinoside, arabinosyl adenine, mercaptopolylysine, vincristine, busulfan, chlorambucil, melphalan (e.g., PAM, a, L-PAM or phenylalanine mustard), mercaptopurine, mitotane. procarbazine hydrochloride, dactinomycin (actinomycin D), daunorubcin hydrochloride, doxorubicin hydrochloride, taxol, mitomycin, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina aparaginase, etoposide-(VP-16), interferon α-2a, interferon α-2b, teniposide (VM-26), vinblastine sulfate (VLB), and arabinosyl. In certain embodiments, the therapeutic may be monoclonal antibody, such as a monoclonal antibody capable of binding to melanoma antigen.

A conjugate labeled with a radionuclide metal, such as ¹⁷⁷Lu, ¹¹¹In, ⁶⁸Ga or ^{99m}Tc, can be administered to a mammal, including human patients or subjects, by, for example, intravenous, subcutaneous or intraperitoneal injection in a pharmaceutically acceptable carrier and/or solution such as salt solutions like isotonic saline. Radiolabeled scintigraphic imaging agents provided by the present invention are provided having a suitable amount of radioactivity. In forming ^{99m}Tc radioactive complexes, it is generally preferred to form radioactive complexes in solutions containing radioactivity at concentrations of from about 0.01 millicurie (mCi) to 100 mCi per mL. Generally, the unit dose to be administered has a radioactivity of about 0.01 mCi to about 100 mCi, preferably 1 mCi to 30 mCi. The solution to be injected at unit dosage is from about 0.01 mL to about 10 mL. The amount of labeled conjugate appropriate for administration is dependent upon the distribution profile of the chosen conjugate in the sense that a rapidly cleared conjugate may need to be administered in higher doses than one that clears less rapidly. In vivo distribution and localization can be tracked by standard scintigraphic techniques at an appropriate time subsequent to administration; typically between thirty minutes and 180 minutes depending upon the rate of accumulation at the target site with respect to the rate of clearance at non-target tissue. For example, after injection of the diagnostic radionuclide-labeled compounds of the invention into the patient, a gamma camera calibrated for the gamma ray energy of the nuclide incorporated in the imaging agent can be used to image areas of uptake of the agent and quantify the amount of radioactivity present in the site. Imaging of the site in vivo can take place in a few minutes. However, imaging can take place, if desired and if the physical half life of the radionuclide permits, hours or even longer, after the radiolabeled peptide is injected into a patient. In most instances, a sufficient amount of the administered dose will accumulate in the area to be imaged within about 0.1 hour to permit the taking of images.

The compounds of the present invention can be administered to a patient alone or as part of a composition that contains other components such as excipients, diluents, radical scavengers, stabilizers, and carriers, all of which are well-known in the art. The compounds can be administered to patients either intravenously or intraperitoneally.

There are numerous advantages associated with the present invention. The compounds made in accordance with some of the embodiments of the present invention form stable, well-defined ^{99m}Tc or ^{186/188}Re labeled compounds. With other embodiments, compounds labeled with ₆₇Ga, ⁶⁸Ga, ¹¹¹n, or ¹⁷⁷Lu are formed. Similar compounds of the invention can also be made by using appropriate chelator frameworks for the respective radiometals, to form stable, well-defined products labeled with ¹⁵³Sm, ⁹⁰Y, ¹⁶⁶Ho, ¹⁰⁵Rh, ¹⁹⁹Au, ¹⁴⁹Pm or other radiometals. The radiolabeled GRP receptor targeting peptides selectively bind to neoplastic cells expressing GRP receptors, and if an agonist is used, become internalized, and are retained in the tumor cells for extended time periods. The radioactive material that does not reach (i.e., does not bind) the cancer cells is preferentially excreted efficiently into the urine with minimal retention of the radiometal in the kidneys.

### a. Methods of Increasing Targeting of the GRP-R

Furthermore, the instant invention includes a method of increasing targeting of a labeled compound of the invention to GRP receptor expressing target tissue as compared to normal (e.g. non-target) GRP receptor expressing tissue. This method comprises administering the appropriate mass of GRP receptor targeting peptide or conjugate, prior to or during administration of labeled compound of the invention. Similarly, the invention includes an improved method of administration of labeled compounds of the invention in which tumor targeting is optimized, comprising administering the appropriate mass dose of GRP receptor targeting peptide or conjugate prior to or during administration of labeled compound of the invention. Such pre- or co-dosing has been found to saturate non-target GRP receptors, decreasing their ability to compete with GRP receptors on tumor tissue.

It has previously been demonstrated that it can be beneficial to pre- or co-dose with increased masses of an active ingredient to occupy circulating binding sites or binding sites on normal (e.g., non-target) tissue which would otherwise compete with the target tissue binding sites for the active ingredient. The goal of such pre-dosing or co-dosing is to increase targeting to and uptake in the target tissue. For instance, unlabeled anti-CD20 antibodies have been administered prior to administration of radioactive anti-CD20 antibodies to occupy binding sites on circulating cells in an attempt to improve visualization of disease sites. In another example, increasing masses of cold somatostatin have been administered in an attempt to saturate the binding sites of normal tissue and thus improve visualization of disease.

No circulating GRP receptor targeting peptide binding proteins have been identified to date. However, there are some normal tissues in the body that express appreciable levels of GRP receptors, which may compete with GRP receptor expressing target tissue. Increasing mass dose would be expected to saturate the binding sites in such tissues. However, it has been unexpectedly found that when mass doses of compounds of the invention are administered, the behaviour of GRP receptor-expressing tumor tissue is different from that of GRP receptor-expressing normal tissues. Specifically, while normal tissue expressing GRP receptors exhibit the expected saturation by the mass dose and thus decreased uptake of labeled compounds of the invention, GRP expressing tumor tissue is unexpectedly resistant to saturation as the mass dose is increased, retaining the ability to bind labeled compounds of the invention. The difference in response between the normal tissue and the tumor tissue may be a reflection of the escape from control of the tumor tissues. Thus this beneficial effect is most likely to occur in those cases where the binding site for a regulatory peptide or compound which is normally closely controlled by the physiology of the normal tissue escapes such control when present in tumor tissue, as in for example the case of GRP receptor expressing tumor tissue.

Thus, in order to optimize tumor targeting, an appropriate mass dose of compound of the invention may be administered prior to or during administration of the labeled compounds of the invention. It should be noted that while administration of a mass dose of unlabeled compounds of the invention is preferred, any active peptide that binds to and interacts with the GRP receptor may be used, whether or not conjugated to a linker and/or a metal chelator or detectable label and whether or not labeled. Preferably, a mass dose of a GRP receptor agonist is used and more preferably it is conjugated to a linker and/or a metal chelator or detectable label, such as those disclosed herein. Most preferably a mass dose of a compound of the invention is administered. While use of unlabeled compounds is preferred for some embodiments, the mass dose may include labeled compound. Indeed, for co-administration applications, administration of a single dose which includes the mass dose and the diagnostic or therapeutic dose of the labeled compound is preferred. In this instance, a labeled, but low specific activity dose is administered which delivers both the appropriate mass dose of unlabeled compound as well as the diagnostically or therapeutically useful dose of labeled compound.

The appropriate mass dose will depend on the specifics of the patient and application, but selection of such dose is within the skill in the art. Useful mass doses are in the range of about 1 to about 20 μg/m² and preferred doses are in the range of about 2 to about 10 μg/m². Where the mass dose is given before the labeled compound of the invention (e.g. pre-dosing) the mass dose is preferably administered no more than about 60 minutes before the diagnostic or therapeutic dose.

### b. Optical Imaging, Sonoluminescence, Photoacoustic Imaging and Phototherapy

In accordance with the present invention, a number of optical parameters may be employed to determine the location of a target with in vivo light imaging after injection of the subject with an optically-labeled compound of the invention. Optical parameters to be detected in the preparation of an image may include transmitted radiation, absorption, fluorescent or phosphorescent emission, light reflection, changes in absorbance amplitude or maxima, and elastically scattered radiation. For example, biological tissue is relatively translucent to light in the near infrared (NIR) wavelength range of 650-1000 nm. NIR radiation can penetrate tissue up to several centimeters, permitting the use of compounds of the present invention to image target-containing tissue in vivo. The use of visible and near-infrared (NIR) light in clinical practice is growing rapidly. Compounds absorbing or emitting in the visible, NIR, or long-wavelength (UV-A, >350 nm) region of the electromagnetic spectrum are potentially useful for optical tomographic imaging, endoscopic visualization, and phototherapy.

A major advantage of biomedical optics lies in its therapeutic potential. Phototherapy has been demonstrated to be a safe and effective procedure for the treatment of various surface lesions, both external and internal. Dyes are important to enhance signal detection and/or photosensitizing of tissues in optical imaging and phototherapy. Previous studies have shown that certain dyes can localize in tumors and serve as a powerful probe for the detection and treatment of small cancers (D. A. Bellnier et al., Murine pharmacokinetics and antitumor efficacy of the photodynamic sensitizer 2-[1-hexyloxyethyl]-2-devinyl pyropheophorbide-a, J. Photochem. Photobiol., 1993, 20, pp. 55-61; G. A. Wagnieres et al., In vivo fluorescence spectroscopy and imaging for oncological applications, Photochem. Photobiol., 1998, 68, pp. 603-632; J. S. Reynolds et al., Imaging of spontaneous canine mammary tumors using fluorescent contrast agents, Photochem. Photobiol., 1999, 70, pp. 87-94). All of these listed references are hereby incorporated by reference in their entirety. However, these dyes do not localize preferentially in malignant tissues.

In an exemplary embodiment, the compounds of the invention may be conjugated with photolabels, such as optical dyes, including organic chromophores or fluorophores, having extensive delocalized ring systems and having absorption or emission maxima in the range of 400-1500 nm. The compounds of the invention may alternatively be derivatized with a bioluminescent molecule. The preferred range of absorption maxima for photolabels is between 600 and 1000 nm to minimize interference with the signal from hemoglobin. Preferably, photoabsorption labels have large molar absorptivities, e.g. >10⁵ cm⁻¹M⁻¹, while fluorescent optical dyes will have high quantum yields. Examples of optical dyes include, but are not limited to those described in U.S. Pat. No. 6,641,798, WO 98/18497, WO 98/18496, WO 98/18495, WO 98/18498, WO 98/53857, WO 96/17628, WO 97/18841, WO 96/23524, WO 98/47538, and references cited therein, all hereby incorporated by reference in their entirety. For example, the photolabels may be covalently linked directly to compounds of the invention, such as, for example, compounds comprised of GRP receptor targeting peptides and linkers of the invention. Several dyes that absorb and emit light in the visible and near-infrared region of electromagnetic spectrum are currently being used for various biomedical applications due to their biocompatibility, high molar absorptivity, and/or high fluorescence quantum yields. The high sensitivity of the optical modality in conjunction with dyes as contrast agents parallels that of nuclear medicine, and permits visualization of organs and tissues without the undesirable effect of ionizing radiation. Cyanine dyes with intense absorption and emission in the near-infrared (NIR) region are particularly useful because biological tissues are optically transparent in this region (B. C. Wilson, Optical properties of tissues. Encyclopedia of Human Biology, 1991, 5, 587-597). For example, Indocyanine green, which absorbs and emits in the NIR region has been used for monitoring cardiac output, hepatic functions, and liver blood flow (Y-L. He, H. Tanigami, H. Ueyama, T. Mashimo, and I. Yoshiya, Measurement of blood volume using indocyanine green measured with pulse-spectrometry: Its reproducibility and reliability. Critical Care Medicine, 1998, 26(8), 1446-1451; J. Caesar, S. Shaldon, L. Chiandussi, et al., The use of Indocyanine green in the measurement of hepatic blood flow and as a test of hepatic function. Clin. Sci. 1961, 21, 43-57) and its functionalized derivatives have been used to conjugate biomolecules for diagnostic purposes (R. B. Mujumdar, L. A. Ernst, S. R. Mujumdar, et al., Cyanine dye labeling reagents: Sulfoindocyanine succinimidyl esters. Bioconjugate Chemistry, 1993, 4(2), 105-111; Linda G. Lee and Sam L. Woo. “N-Heteroaromatic ion and iminium ion substituted cyanine dyes for use as fluorescent labels”, U.S. Pat. No. 5,453,505; Eric Hohenschuh, et al. “Light imaging contrast agents”, WO 98/48846; Jonathan Turner, et al. “Optical diagnostic agents for the diagnosis of neurodegenerative diseases by means of near infra-red radiation”, WO 98/22146; Kai Licha, et al. “In-vivo diagnostic process by near infrared radiation”, WO 96/17628; Robert A. Snow, et al., Compounds, WO 98/48838, U.S. Pat. No. 6,641,798. All of these listed references are hereby incorporated by reference in their entirety.

After injection of the optically-labeled compound, the patient is scanned with one or more light sources (e.g., a laser) in the wavelength range appropriate for the photolabel employed in the agent. The light used may be monochromatic or polychromatic and continuous or pulsed. Transmitted, scattered, or reflected light is detected via a photodetector tuned to one or multiple wavelengths to determine the location of target-containing tissue (e.g., tissue containing GRP) in the subject. Changes in the optical parameter may be monitored over time to detect accumulation of the optically-labeled reagent at the target site (e.g. the tumor or other site with GRP receptors). Standard image processing and detecting devices may be used in conjunction with the optical imaging reagents of the present invention.

The optical imaging reagents described above may also be used for acousto-optical or sonoluminescent imaging performed with optically-labeled imaging agents (see, U.S. Pat. No. 5,171,298, WO 98/57666, and references therein). In acousto-optical imaging, ultrasound radiation is applied to the subject and affects the optical parameters of the transmitted, emitted, or reflected light. In sonoluminescent imaging, the applied ultrasound actually generates the light detected. Suitable imaging methods using such techniques are described in WO 98/57666.

Various imaging techniques and reagents are described in U.S. Pat. Nos. 6,663,847, 6,656,451, 6,641,798, 6,485,704, 6,423,547, 6,395,257, 6,280,703, 6,277,841, 6,264,920, 6,264,919, 6,228,344, 6,217,848, 6,190,641, 6,183,726, 6,180,087, 6,180,086, 6,180,085, 6,013,243, and published U.S. Patent Applications 2003185756, 20031656432, 2003158127, 2003152577, 2003143159, 2003105300, 2003105299, 2003072763, 2003036538, 2003031627, 2003017164, 2002169107, 2002164287, and 2002156117, all of which are hereby incorporated by reference.

### c. Radiotherapy

Radioisotope therapy involves the administration of a radiolabeled compound in sufficient quantity to damage or destroy the targeted tissue. After administration of the compound (by e.g., intravenous, subcutaneous, or intraperitonal injection), the radiolabeled pharmaceutical localizes preferentially at the disease site (in this instance, tumor tissue or other tissue that expresses the pertinent GRP receptor). Once localized, the radiolabeled compound then damages or destroys the diseased tissue with the energy that is released during the radioactive decay of the isotope that is administered. As discussed herein, the invention also encompasses use of radiotherapy in combination with chemotherapy (or in combination with any other appropriate therapeutic agent).

The design of a successful radiotherapeutic involves several critical factors:
1. selection of an appropriate targeting group to deliver the radioactivity to the disease site;
2. selection of an appropriate radionuclide that releases sufficient energy to damage that disease site, without substantially damaging adjacent normal tissues; and
3. selection of an appropriate combination of the targeting group and the radionuclide without adversely affecting the ability of this conjugate to localize at the disease site. For radiometals, this often involves a chelating group that coordinates tightly to the radionuclide, combined with a linker that couples said chelate to the targeting group, and that affects the overall biodistribution of the compound to maximize uptake in target tissues and minimize uptake in normal, non-target organs.

Radiotherapeutic agents may contain a chelated 3+ metal ion from the class of elements known as the lanthanides (elements of atomic number 57-71) and their analogs (i.e. M³⁺ metals such as yttrium and indium). Typical radioactive metals in this class include the isotopes 90-Yttrium, 111-Indium, 149-Promethium, 153-Samarium, 166-Dysprosium, 166-Holmium, 175-Ytterbium, and ¹⁷⁷-Lutetium. All of these metals (and others in the lanthanide series) have very similar chemistries, in that they remain in the +3 oxidation state, and prefer to chelate to ligands that bear hard (oxygen/nitrogen) donor atoms, as typified by derivatives of the well known chelate DTPA (diethylenetriaminepentaacetic acid) and polyaza-polycarboxylate macrocycles such as DOTA (1,4,7,10-tetrazacyclododecane-N,N′,N″,N′″-tetraacetic acid and its close analogs. The structures of these chelating ligands, in their fully deprotonated form are shown below.

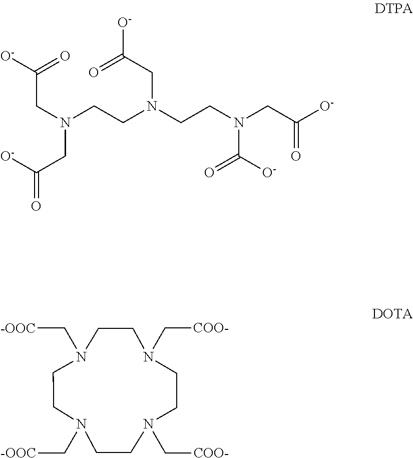

These chelating ligands encapsulate the radiometal by binding to it via multiple nitrogen and oxygen atoms, thus preventing the release of free (unbound) radiometal into the body. This is important, as in vivo dissociation of 3⁺ radiometals from their chelate can result in uptake of the radiometal in the liver, bone and spleen [Brechbiel M W, Gansow O A, “Backbone-substituted DTPA ligands for ⁹⁰Y radioimmunotherapy”, Bioconj. Chem. 1991; 2: 187-194; L1, WP, Ma D S, Higginbotham C, Hoffman T, Ketring A R, Cutler C S, Jurisson, S S, “Development of an in vitro model for assessing the in vivo stability of lanthanide chelates.” Nucl. Med. Biol. 2001; 28(2): 145-154; Kasokat T, Urich K. Arzneim.-Forsch, “Quantification of dechelation of gadopentetate dimeglumine in rats”. 1992; 42(6): 869-76]. Unless one is specifically targeting these organs, such non-specific uptake is highly undesirable, as it leads to non-specific irradiation of non-target tissues, which can lead to such problems as hematopoietic suppression due to irradiation of bone marrow.

For radiotherapy applications any of the chelators for therapeutic radionuclides disclosed herein may be used. However, forms of the DOTA chelate [Tweedle M F, Gaughan G T, Hagan J T, “1-Substituted-1,4,7-triscarboxymethyl-1,4,7,10-tetraazacyclododecane and analogs.” U.S. Pat. No. 4,885,363, Dec. 5, 1989] are particularly preferred, as the DOTA chelate is expected to de-chelate less in the body than DTPA or other linear chelates. Compounds L64 and L70 (when labeled with an appropriate therapeutic radionuclide) are particularly preferred for radiotherapy.

General methods for coupling DOTA-type macrocycles to targeting groups through a linker (e.g. by activation of one of the carboxylates of the DOTA to form an active ester, which is then reacted with an amino group on the linker to form a stable amide bond), are known to those skilled in the art. (See e.g. Tweedle et al. U.S. Pat. No. 4,885,363). Coupling can also be performed on DOTA-type macrocycles that are modified on the backbone of the polyaza ring.

The selection of a proper nuclide for use in a particular radiotherapeutic application depends on many factors, including:

Physical half-life—This should be long enough to allow synthesis and purification of the radiotherapeutic construct from radiometal and conjugate, and delivery of said construct to the site of injection, without significant radioactive decay prior to injection. Preferably, the radionuclide should have a physical half-life between about 0.5 and 8 days.
Energy of the emission(s) from the radionuclide—For radiotherapy applications, radionuclides that are particle emitters (such as alpha emitters, beta emitters and Auger electron emitters) are particularly useful, as they emit highly energetic particles that deposit their energy over short distances, thereby producing highly localized damage. Beta emitting radionuclides are particularly preferred, as the energy from beta particle emissions from these isotopes is deposited within 5 to about 150 cell diameters. Radiotherapeutic agents prepared from these nuclides are capable of killing diseased cells that are relatively close to their site of localization, but cannot travel long distances to damage adjacent normal tissue such as bone marrow.
Specific activity (i.e. radioactivity per mass of the radionuclide)-Radionuclides that have high specific activity (e.g. generator produced 90-Y, 111-In, 177-Lu) are particularly preferred. The specific activity of a radionuclide is determined by its method of production, the particular target that is used to produce it, and the properties of the isotope in question.

Many of the lanthanides and lanthanoids include radioisotopes that have nuclear properties that make them suitable for use as radiotherapeutic agents, as they emit beta particles or Auger electrons. Some of these are listed in Table 6.

TABLE 6

| | | | | Approximate |
|---|---|---|---|---|
| | | Max β- | | range of β- |
| | Half-Life | energy | Gamma energy | particle |
| Isotope | (days) | (MeV) | (keV) | (cell diameters) |
| ¹⁴⁹Pm | 2.21 | 1.1 | 286 | 60 |
| ¹⁵³Sm | 1.93 | 0.69 | 103 | 30 |
| ¹⁶⁶Dy | 3.40 | 0.40 | 82.5 | 15 |
| ¹⁶⁶Ho | 1.12 | 1.8 | 80.6 | 117 |
| ¹⁷⁵Yb | 4.19 | 0.47 | 396 | 17 |
| ¹⁷⁷Lu | 6.71 | 0.50 | 208 | 20 |
| ⁹⁰Y | 2.67 | 2.28 | — | 150 |
| ¹¹¹In | 2.81 | Auger electron | 173, 247 | <5 μm |
| | | emitter | | |

Pm: Promethium,
Sm: Samarium,
Dy: Dysprosium,
Ho: Holmium,
Yb: Ytterbium,
Lu: Lutetium,
Y: Yttrium,
In: Indium

Methods for the preparation of radiometals such as beta-emitting lanthanide radioisotopes are known to those skilled in the art, and have been described elsewhere [e.g., Cutler C S, Smith C J, Ehrhardt G J.; Tyler T T, Jurisson S S, Deutsch E. “Current and potential therapeutic uses of lanthanide radioisotopes.” Cancer Biother. Radiopharm. 2000; 15(6): 531-545]. Many of these isotopes can be produced in high yield for relatively low cost, and many (e.g. ⁹⁰Y, ¹⁴⁹Pm, ¹⁷⁷Lu) can be produced at close to carrier-free specific activities (i.e. a high percentage of atoms are radioactive). Since non-radioactive atoms can compete with their radioactive analogs for binding to receptors on the target tissue, the use of high specific activity radioisotope is important, to allow delivery of as high a dose of radioactivity to the target tissue as possible.

Radiotherapeutic derivatives of the invention containing beta-emitting isotopes of rhenium (¹⁸⁶—Re and ¹⁸⁸—Re) are also particularly preferred.

The present invention provides radiotherapeutic agents that satisfy all three of the above criteria, through proper selection of targeting group, radionuclide, metal chelate and linker. The compounds of the invention, which, as explained in more detail in the Examples, show greater specificity and higher uptake in tumors in vivo than compounds without the novel linkers disclosed herein, exhibit an improved ability to target GRP receptor-expressing tumors and thus to image or deliver radiotherapy to these tissues. Indeed, as shown in the Examples, radiotherapy is more effective (and survival time increased) using compounds of the invention.

Moreover, as shown in the Examples, compounds of the invention are particularly useful in the treatment of prostate cancer, including bone or soft tissue metastases of prostate cancer and in both hormone sensitive and hormone refractory prostate cancer.

Compounds of the invention, particularly radiolabeled L70, are also useful in methods of delaying progression and decreasing vascular permeability of prostate cancer, particularly hormone sensitive prostate cancer. Indeed, as shown in the Examples, compounds of the invention may delay time to progression by about 100%. This is significant particularly as some drugs have been approved which decrease time to progression by as little as 15%. The compounds of the invention are also useful in facilitating combination therapy of hormone sensitive prostate cancer. Combination therapy includes administration of a compound of the invention as well as another substance useful in treating prostate cancer such, as for example, a chemotherapeutic. Compounds of the invention facilitate such combination therapy by, for example, normalizing the blood flow to tumors, facilitating the delivery of the additional therapeutic agent.

d. Methods of Assessing the Therapeutic Utility and Response to Drugs Targeted to Receptors which Crosstalk with GRP-R (e.g. ⁶⁸Ga-AMBA: PET Imaging of GRP-R as a Sentinel Receptor)

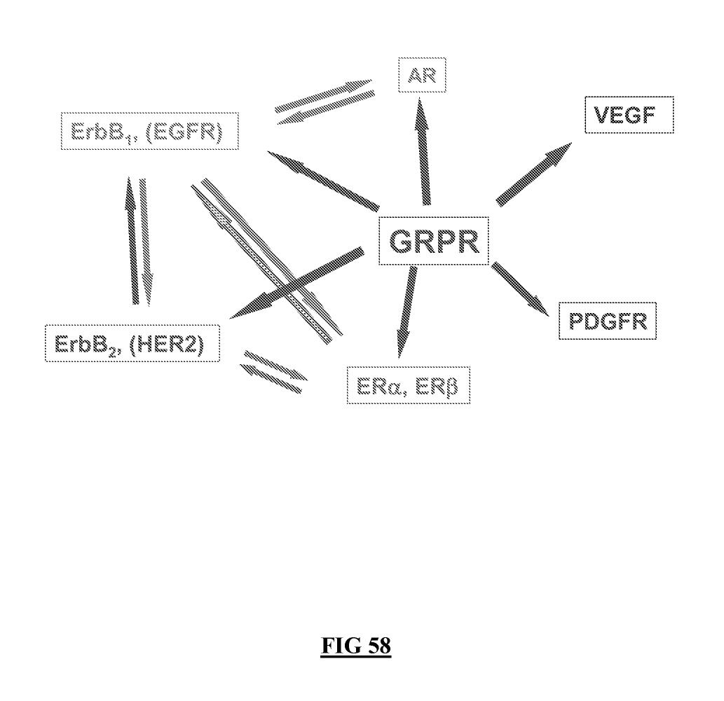

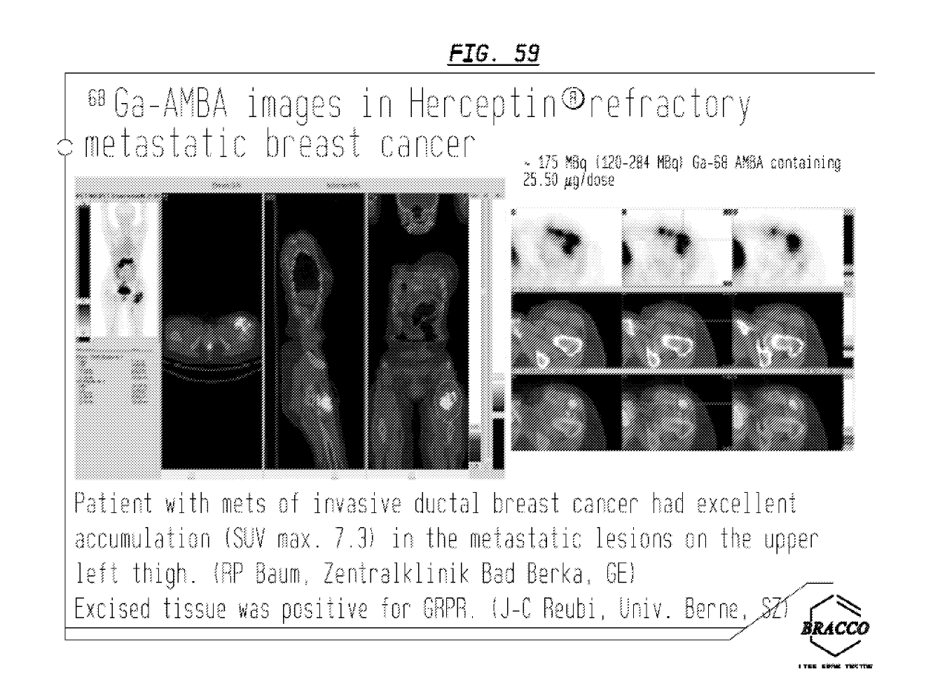

The present invention also provides a method of examining crosstalk with GRP receptors, specifically cross talk in the RTK or “other target” to GRPR direction, for a broad spectrum of solid human tumors that express GRPR (primaries and metastases) including, but not limited to breast and prostate cancer. Referring now to FIG. 58, known cross-talk involving the GRP-R with other receptors used in cancer therapeutics is depicted. These instances of crosstalk describe the effect on the ‘peripheral’ receptors when the ‘central’ GRP receptor is targeted. Suprisingly, we now find that crosstalk can operate in the opposite direction, that is when the activity of the ‘peripheral’ receptor is changed due to an intervention this changes the activity of the ‘central’ GRP receptor. The GRP receptor that is targeted by the molecules of the present invention engages in cross-talk with a wide variety of other cancer receptors. Cross talk means that there is communication between the ‘central’ GRP receptor and one or more of the ‘peripheral’ receptors. A ‘peripheral’ receptor exhibiting crosstalk with the GRP receptor will, on a change in activity as a result of an intervention directed towards it, lead to a change in activity of the GRP receptor. When this occurs consistently and to a sufficient degree, the GRP receptor can be used to determine the status of the other, peripheral receptor. Each of these receptors is quite important in cancer therapy due to targeted drugs being associated with each receptor. Without being bound to a particular theory, these receptors may also have an influence on the GRP-R, and thus GRP-R targeted compounds of the invention may be used to determine if and when one or more of the other receptors is active in the patient's tumor or tumors, allowing the oncologist to decide from the image the degree to which the drug targeted to that receptor is being effective in that tumor.

Specifically, the invention permits assessing the effect treatment with any one of a broad class of therapeutics targeted to receptors that crosstalk with GRP-R, such as RTK receptors or the estrogen receptor (e.g. RTK inhibitors, estrogen inhibitors), administered under normal clinical conditions (dose and schedule), may have on the function of such receptors (e.g. RTK receptors or the estrogen receptor) as detected by changes in the expression of the GRP receptor specific signal with which they exhibit crosstalk. Imaging the GRP-R receptor provides information on the response to treatment (pre/post) of drugs (e.g. Iressa, Herceptin, and Tamoxifen) targeted to other receptors which crosstalk with GRP-R and is particularly helpful where response rates are low. For example, in breast cancer, Herceptin has a 9% response (30% have Her2neu, 30% who are treated respond). Similarly, Avastin has a 10% response in metastatic colon cancer and Erbitux has an 11-14% response in metastatic colon cancer.

The invention provides a functional indication of the anticipated response to therapy with such drugs via an increase, decrease, or no change in the GRP receptor specific signal activity in vivo.

The invention also provides a method of screening new drugs which target peripheral receptors which crosstalk with GRP-R for changes in the activity of the GRP receptor family in vitro using a radiolabeled or otherwise detectable labeled agonist or antagonist of the GRP receptors.

The invention also provides a method of imaging the activity of the GRP receptor family in vivo using a radiolabeled agonist or antagonist of the GRP receptors to monitor the therapeutic effect of a drug.

Such methods utilize a GRP-R binding ligand of the invention defined herein, wherein M is a chelator complexed with a radionuclide detectable by scintigraphy or PET imaging or is or a moiety that contains a radiolabeled halogen such as F-18, ¹²³I—, ¹²⁴I— or ¹³¹I— and M-N—O—P-Q are as defined herein, to image the GRP-R to monitor therapeutic progress of a drug which targets a receptor which crosstalks with GRPR.

In a preferred embodiment, the method envisages using the GRPR-binding ligand of the invention, AMBA (referred to herein interchangeably as L70 or AMBA), preferably labeled with ⁶⁸Ga (referred to herein interchangeably as ⁶⁸Ga-L70 or ⁶⁸Ga-AMBA) or F-18, ¹²³I—, ¹²⁴I—, ¹³¹I—, or ^{99m}Tc-labelled to image GRP receptors to monitor therapeutic response to a drug which targets a receptor which crosstalks with GRPR. In a particularly preferred embodiment, ⁶⁸Ga-AMBA is used in such methods.

In a preferred embodiment the imaging of the GRP-R is longitudinal: e.g. imaging occurs prior to initial treatment (to obtain a baseline value), during treatment (to predict and monitor response, and detect when a shift in the tumor population may warrant a change of therapeutic), and at the end of or post treatment (to aid in determination of efficacy, next treatment steps and to predict or look for relapse).

Prior imaging occurs up to about 30 days before commencement of treatment, preferably up to 15 days before and ideally up to 7 days before. Imaging after commencement of treatment occurs at the end of the first course of treatment, preferably within 15 days of start of treatment and ideally up to 7 days after commencement. Imaging at the end of treatment occurs after a planned course or courses of treatment and periodically thereafter or on suspicion of recurrence.

An imaging dose of about 3-12 mCi (111-444 MBq), preferably −3-5 mCi (110-185 MBq) of the radiolabeled compound (such as ⁶⁸Ga-AMBA or ⁶⁷Ga-AMBA) with a mass dose of up to about 50 μg of peptide administered either as a bolus or by slow infusion over minutes can be used.

Imaging of the radiolabeled compound of the invention is performed at an appropriate interval after administration. For example for ⁶⁸Ga, imaging is performed about 0.5-2 h after administration of ⁶⁸Ga labeled material using an imaging technique called Positron Emission Tomography (PET) and devices well known to those in the field. For example, those described by R. P. Baum at the European Association of Nuclear Medicine Meeting in Copenhagen in 2007. Alternatively, imaging of non positron emitting radionuclides can be performed with a SPECT device using techniques well known to those in the field. For longer lived radionuclides imaging can be performed at longer times consistent with their physical half life.

The anticipated responses depend on the particular relationship between the target of the therapeutic drug and the GRP receptor and include, but are not limited to, a monotonic increase, decrease, or no change in GRP receptor specific signal as detected by imaging.

For example, a decrease in activity may be caused by effective targeting with the therapeutic drug leading to a reduction in activity of the GRP receptor by crosstalk and inability of the tumour tissue to switch to GRPR for support/rescue; or it may indicate that treatment is not effective and therefore there is no selective pressure on the tumour tissue to switch to GRPR.

Or, an increase in activity may be caused by the cell or tumor initiating survival mechanisms that include the GRP receptor. Such mechanisms may presage the ultimate death of the cell or an attempt to evade destruction (switch to GRP for continued growth, or boosting the targeted pathway by for example transactivation of the targeted pathway by GRPR).

Lastly, no change in GRPR could imply that the therapeutic treatment is efficacious and that GRPR is not an alternative pathway for rescue available to the cell; or that the treatment is not successful, and therefore there is no need to transactivate GRPR; or that GRP is mediating rescue, but there is no need for increased expression of GRPR.

The anticipated response depends both on the therapeutic drug or combination used and on the cancer cell being targeted and will be made clearer by the examples cited. Monitoring these changes in function of the GRP family of receptors is most useful when they occur before overt changes in the tumor are seen i.e. before there is an observable reduction in tumor cell proliferation and/or an increase in tumor cell loss observable as a lessening in tumor growth rate or indeed a reduction in tumor size.

The following tables summarize the possible outcomes of imaging the GRP-R with a compound of the invention (preferably Ga-AMBA) alone or in combination with ¹⁸F-FDG.

TABLE 7
Treatment Decision Matrix (Ga-AMBA only)

| | |
|---|---|
| ⁶⁸Ga-AMBA ↑ | Indicates crosstalk, possible options: |
| | SD or PD; either last gasp, or rescue: image again in a |
| | shorter timeframe to see if GRP receptor specific |
| | signal continues to increase, which could indicate |
| | that the tumor is using GRPR to evade death (rescue) |
| | and a change in treatment would be recommend; |
| | if the signal declines with repeated imaging then the |
| | initial spike was a last gasp attempt and no |
| | change of treatment would be recommended. |
| ⁶⁸Ga-AMBA ↓ | Indicates crosstalk, possible options:: |
| | PR, treatment is working, no change in treatment |
| | course recommended. |
| | SD, possibly PR; no apparent rescue; image again in a |
| | shorter timeframe to see if GRP receptor specific |
| | signal continues to decrease. |
| | Additional targeting of GRPR may be beneficial. |
| ⁶⁸Ga-AMBA [symbol] | [symbol] apparent crosstalk or no effect of therapeutic drug |

KEY:
increase = ↑,
decrease = ↓,
No change = [symbol]
PR = partial response or better
SD = stable disease
PD = Progressive disease [symbol]
indicates data missing or illegible when filed

TABLE 8
Treatment Decision Matrix, Ga-AMBA combined with ¹⁸F-FDG

| | ¹⁸F-FDG ↑ | ¹⁸F-FDG ↓ | ¹⁸F-FDG [symbol] |
|---|---|---|---|
| ⁶⁸Ga-AMBA ↑ | F↑ A↑ | F↓ A↑ | F [symbol] A↑ |
| | Indicates crosstalk: | Indicates crosstalk: | Indicates crosstalk: |
| | PD, treatment not working, | PR, treatment is working, | SD or PD, either last gasp, or |
| | tumor may be using GRPR | GRPR is not rescuing | rescue. Image again in a short |
| | to evade death, recommend | tumor, no change in | timeframe to see if FDG is stab [symbol] |
| | change in treatment course. | treatment recommended. | increasing or decreasing; GRP [symbol] |
| | | | may be rescuing, and change [symbol] |
| | | | treatment may be necessary. |
| ⁶⁸Ga-AMBA ↓ | F↑ A↓ | F↓ A↓ | F [symbol] A↓ |
| | Indicates crosstalk: | Indicates crosstalk: | Indicates crosstalk: |
| | PD, but it is NOT essential | PR, treatment is working, | SD, possibly PR, no apparent |
| | (no rescue); treatment not | no change in treatment | rescue; image again in a short [symbol] |
| | working, and change | course recommended. | timeframe to see if FDG contin [symbol] |
| | treatment course is | | to be stable, increases or |
| | recommended. | | decreases. Targeting GRP as [symbol] |
| | | | may be beneficial. |
| ⁶⁸Ga-AMBA [symbol] | F↑ A [symbol] | F↓ A [symbol] | F [symbol] A [symbol] |
| | No apparent crosstalk: | No apparent crosstalk: | No apparent crosstalk: |
| | PD, change treatment | PR, no change in | SD, treatment is working, no |
| | course recommended. | treatment course | change in treatment course |
| | | recommended. | recommended. |

KEY:
increase = ↑,
decrease = ↓,
No change = [symbol]
PR = partial response or better
SD = stable disease
PD = Progressive disease
F = ¹⁸F-FDG
A = ⁶⁸Ga-AMBA [symbol]
indicates data missing or illegible when filed

As explained in more detail in the Examples, experiments herein show that drug treatment not targeted at the GRP receptor modulates GRP-R uptake of ¹⁷⁷Lu-AMBA (and by inference ^{67/68}Ga-AMBA) on a viable cell basis in vitro in cell cultures and in vivo in mice. Such experiments demonstrate the crosstalk between drugs targeted to peripheral crosstalking receptors and the GRP receptor in prostate and breast cancer cell lines.

Suprisingly, the Examples also establish that the signal detected using ¹⁷⁷Lu-AMBA to measure the GRP-R activity is different to that of FDG. For example, dasatinib treatment pf PC-3 prostate cancer cells increases ¹⁷⁷Lu-AMBA uptake by up to 76 percent but decreases NBDG, which is a fluorescent analogue of 2 deoxyglucose and ¹⁸F-Fluorodeoxyglucose, by a similar amount. In general, an increase in signal is more desirable than a decrease in signal because of an increased dynamic range.

Thus, the Examples demonstrate the ability of GRP-R activity (and thus imaging of the GRP-R) to document the effect of drugs targeted to receptors which crosstalk with GRP-R such as, for example, the estrogen receptor, the Src receptor family, various RTK receptors, etc). Further, the Examples, show that compounds of the invention such as Ga-AMBA, are better suited than ^{s}F-FDG to monitor therapeutic progress.

### 6. Dosages and Additives

Proper dose schedules for the compounds of the present invention are known to those skilled in the art. The compounds can be administered using many methods which include, but are not limited to, a single or multiple IV or IP injections. For radiopharmaceuticals, one administers a quantity of radioactivity that is sufficient to permit imaging or, in the case of radiotherapy, to cause damage or ablation of the targeted GRP-R bearing tissue, but not so much that substantive damage is caused to non-target (normal tissue). The quantity and dose required for scintigraphic imaging is discussed supra. The quantity and dose required for radiotherapy is also different for different constructs, depending on the energy and half-life of the isotope used, the degree of uptake and clearance of the agent from the body and the mass of the tumor. In general, doses can range from a single dose of about 30-50 mCi to a cumulative dose of up to about 3 Curies.

As explained herein the administration of an appropriately selected mass dose can decrease the proportion of the administered dose of labeled compound of the invention in normal tissues having functioning GRP receptors, thus improving the perspicuity of the tumor signal and/or increasing the dose of a therapeutic radionuclide in the tumor.

For optical imaging compounds, dosages sufficient to achieve the desired image enhancement are known to those skilled in the art and may vary widely depending on the dye or other compound used, the organ or tissue to be imaged, the imaging equipment used, etc.

The compositions of the invention can include physiologically acceptable buffers, and can require radiation stabilizers to prevent radiolytic damage to the compound prior to injection. Radiation stabilizers are known to those skilled in the art, and may include, for example, para-aminobenzoic acid, ascorbic acid, gentistic acid and the like.

A single, or multi-vial kit that contains all of the components needed to prepare the diagnostic or therapeutic agents of this invention is an integral part of this invention. In the case of radiopharmaceuticals, such kits will often include all necessary ingredients except the radionuclide.

For example, a single-vial kit for preparing a radiopharmaceutical of the invention preferably contains a chelator/linker/targeting peptide conjugate of the formula M-N—O—P-G, a source of stannous salt (if reduction is required, e.g., when using technetium), or other pharmaceutically acceptable reducing agent, and is appropriately buffered with pharmaceutically acceptable acid or base to adjust the pH to a value of about 3 to about 9. The quantity and type of reducing agent used will depend highly on the nature of the exchange complex to be formed. The proper conditions are well known to those that are skilled in the art. It is preferred that the kit contents be in lyophilized form. Such a single vial kit may optionally contain labile or exchange ligands such as glucoheptonate, gluconate, mannitol, malate, citric or tartaric acid and can also contain reaction modifiers such as diethylenetriamine-pentaacetic acid (DPTA), ethylenediamine tetraacetic acid (EDTA), or a, β, or γ-cyclodextrin that serve to improve the radiochemical purity and stability of the final product. The kit may also contain stabilizers, bulking agents such as mannitol, that are designed to aid in the freeze-drying process, and other additives known to those skilled in the art.

A multi-vial kit preferably contains the same general components but employs more than one vial in reconstituting the radiopharmaceutical. For example, one vial may contain all of the ingredients that are required to form a labile Tc(V) complex on addition of pertechnetate (e.g. the stannous source or other reducing agent). Pertechnetate is added to this vial, and after waiting an appropriate period of time, the contents of this vial are added to a second vial that contains the chelator and targeting peptide, as well as buffers appropriate to adjust the pH to its optimal value. After a reaction time of about 5 to 60 minutes, the complexes of the present invention are formed. It is advantageous that the contents of both vials of this multi-vial kit be lyophilized. As above, reaction modifiers, exchange ligands, stabilizers, bulking agents, etc. may be present in either or both vials.

### General Preparation of Compounds

The compounds of the present invention can be prepared by various methods depending upon the selected chelator. The peptide portion of the compound can be most conveniently prepared by techniques generally established and known in the art of peptide synthesis, such as the solid-phase peptide synthesis (SPPS) approach. Because it is amenable to solid phase synthesis, employing alternating FMOC protection and deprotection is the preferred method of making short peptides. Recombinant DNA technology is preferred for producing proteins and long fragments thereof.

Solid-phase peptide synthesis (SPPS) involves the stepwise addition of amino acid residues to a growing peptide chain that is linked to an insoluble support or matrix, such as polystyrene. The C-terminal residue of the peptide is first anchored to a commercially available support with its amino group protected with an N-protecting agent such as a t-butyloxycarbonyl group (Boc) or a fluorenylmethoxycarbonyl (Fmoc) group. The amino protecting group is removed with suitable deprotecting agents such as TFA in the case of Boc or piperidine for Fmoc and the next amino acid residue (in N-protected form) is added with a coupling agent such as diisopropylcarbodiimide (DIC). Upon formation of a peptide bond, the reagents are washed from the support. After addition of the final residue, the peptide is cleaved from the support with a suitable reagent such as trifluoroacetic acid (TFA) or hydrogen fluoride (HF).

Alternative Preparation of the Compounds via Segment Coupling

### The compounds of the invention may also be prepared by the process known in the art as segment coupling or fragment condensation (Barlos, K. and Gatos, D.; 2002 “Convergent Peptide Synthesis” in Fmoc Solid Phase Synthesis—A Practical Approach; Eds. Chan, W. C. and White, P. D.; Oxford University Press, New York; Chap. 9, pp 215-228). In this method, segments of the peptide usually in side-chain protected form, are prepared separately by either solution phase synthesis or solid phase synthesis or a combination of the two methods. The choice of segments is crucial and is made using a division strategy that can provide a manageable number of segments whose C-terminal residues and N-terminal residues are projected to provide the cleanest coupling in peptide synthesis. The C-terminal residues of the best segments are either devoid of chiral alpha carbons (glycine or other moieties achiral at the carbon ∝ to the carboxyl group to be activated in the coupling step) or are compromised of amino acids whose propensity to racemization during activation and coupling is lowest of the possible choices. The choice of N-terminal amino acid for each segment is based on the ease of coupling of an activated acyl intermediate to the amino group. Once the division strategy is selected the method of coupling of each of the segments is chosen based on the synthetic accessibility of the required intermediates and the relative ease of manipulation and purification of the resulting products (if needed). The segments are then coupled together, both in solution, or one on solid phase and the other in solution to prepare the final structure in fully or partially protected form.

The protected target compound is then subjected to removal of protecting groups, purified and isolated to give the final desired compound. Advantages of the segment coupling approach are that each segment can be purified separately, allowing the removal of side products such as deletion sequences resulting from incomplete couplings or those derived from reactions such as side-chain amide dehydration during coupling steps, or internal cyclization of side-chains (such as that of Gln) to the alpha amino group during deprotection of Fmoc groups. Such side products would all be present in the final product of a conventional resin-based ‘straight through’ peptide chain assembly whereas removal of these materials can be performed, if needed, at many stages in a segment coupling strategy. Another important advantage of the segment coupling strategy is that different solvents, reagents and conditions can be applied to optimize the synthesis of each of the segments to high purity and yield resulting in improved purity and yield of the final product. Other advantages realized are decreased consumption of reagents and lower costs.

### ExampleS

The following are provided as examples of different methods which can be used to prepare various compounds of the present invention. Within each example, there are compounds identified in single bold capital letter (e.g., A, B, C), which correlate to the same labeled corresponding compounds in the drawings identified.

### General Experimental

### A. Definitions of Additional Abbreviations Used

The following common abbreviations are used throughout this specification:
1,1-dimethylethoxycarbonyl (Boc or Boc);
9-fluorenylmethyloxycarbonyl (Fmoc);
allyloxycarbonyl (Aloc);
   1-hydroxybenozotriazole (HOBt or HOBT);
N,N′-diisopropylcarbodiimide (DIC);
N-methylpyrrolidinone (NMP);
acetic anhydride (Ac₂O);
(4,4-dimethyl-2,6-dioxocyclohex-1-ylidene)-3-methylbutyl (iv-Dde); trifluoroacetic acid (TFA);
Reagent B (TFA:H₂O:phenol:triisopropylsilane, 88:5:5:2); diisopropylethylamine (DIEA);
O-(1H-benzotriazole-1-yl)-N,N,N′,N′-tetramethyluronium hexafluorophosphate (HBTU);
O-(7-azabenzotriazol-1-yl)-1,1,3,3-tetramethyluronium hexafluorophosphate (HATU);
N-hydroxysuccinimide (NHS);
solid phase peptide synthesis (SPPS);
dimethylsulfoxide (DMSO);
dichloromethane (DCM);
dimethylformamide (DMF);
dimethylacetamide (DMA);
   1,4,7,10-tetraazacyclotetradecane-1,4,7,10-tetraacetic acid (DOTA);
Triisopropylsilane (TIPS);
   1,4,7,10-tetraazacyclotetradecane-1,4,7,10-tetraacetic acid (DOTA)
(1R)-1-[1,4,7,10-tetraaza-4,7,10-tris(carboxymethyl)cyclododecyl]
ethane-1,2-dicarboxylic acid (CMDOTA);
fetal bovine serum (FBS);
human serum albumin (HSA);
human prostate cancer cell line (PC3);
isobutylchloroformate (IBCF);
tributyl amine (TBA);
radiochemical purity (RCP); and
high performance liquid chromatography (HPLC).

### B. Materials

The Fmoc-protected amino acids used were purchased from Nova-Biochem (San Diego, Calif., USA), Advanced Chem Tech (Louisville, Ky., USA), Chem-Impex International (Wood Dale Ill., USA), and Multiple Peptide Systems (San Diego, Calif., USA). Other chemicals, reagents and adsorbents required for the syntheses were procured from Aldrich Chemical Co. (Milwaukee, Wis., USA) and VWR Scientific Products (Bridgeport, N.J., USA). Solvents for peptide synthesis were obtained from Pharmco Co. (Brookfield Conn., USA). Columns for HPLC analysis and purification were obtained from Waters Co. (Milford, Mass., USA). Experimental details are given below for those that were not commercially available.

### C. Instrumentation for Peptide Synthesis

Peptides were prepared using an Advanced ChemTech 496 Q MOS synthesizer, an Advanced ChemTech 357 FBS synthesizer and/or by manual peptide synthesis. However the protocols for iterative deprotection and chain extension employed were the same for all.

### D. Automated Synthesis with the Symphony Instrument

### Made by Rainin

The synthesis was run with Symphony Software (Version 3) supplied by Protein Technologies Inc. Novagel TGR resin, with a substitution of 0.25 mmol/g, was used, and each well contained 0.2 g of the resin (50 μmol). The amino acids were dissolved in NMP and the concentration was 0.25M. A 0.25M solution of HBTU and N-Methylmorpholine in DMF was prepared and used for the coupling. All the couplings were carried out for 2.0 h. The cleavage was done outside the machine by transferring the resin to another reaction vessel and using Reagent B as in the manual synthesis.

### E. Instrumentation Employed for Analysis and Purification

Analytical HPLC was performed using a Shimadzu-LC-100A dual pump gradient analytical LC system employing Shimadzu-ClassVP software version 4.1 for system control, data acquisition, and post run processing. Mass spectra were acquired on a Hewlett-Packard Series 1100 MSD mass spectrometer interfaced with a Hewlett-Packard Series 1100 dual pump gradient HPLC system fitted with an Agilent Technologies 1100 series autosampler fitted for either direct flow injection or injection onto a Waters Associates XTerra MS C18 column (4.6 mm×50 mm, 5μ particle, 120 Å pore). The instrument was driven by a HP Kayak workstation using ‘MSD Anyone’ software for sample submission and HP ChemStation software for instrument control and data acquisition. In most cases the samples were introduced via direct injection using a 5 μL injection of sample solution at a concentration of 1 mg/mL and analyzed using positive ion electrospray to obtain m/e and m/z (multiply charged) ions for confirmation of structure. ¹H-NMR spectra were obtained on a Varian Innova spectrometer at 500 MHz. ¹³C-NMR spectra were obtained on the same instrument at 125.73 MHz. Generally the residual ¹H absorption, or in the case of ¹³C-NMR, the ¹³C absorption of the solvent employed, was used as an internal reference; in other cases tetramethylsilane (6=0.00 ppm) was employed. Resonance values are given in δ units. Micro analysis data was obtained from Quantitative Technologies Inc., Whitehouse N.J. Preparative HPLC was performed on a Shimadzu-LC-8A dual pump gradient preparative HPLC system employing Shimadzu-ClassVP software version 4.3 for system control, data acquisition, fraction collection and post run processing.

### F. General Procedures for Peptide Synthesis

Rink Amide-Novagel HL resin (0.6 mmol/g) was used as the solid support.

### G. Coupling Procedure

In a typical experiment, the first amino acid was loaded onto 0.1 g of the resin (0.06 mmol). The appropriate Fmoc-amino acid in NMP (0.25M solution; 0.960 mL) was added to the resin followed by N-hydroxybenzotriazole (0.5M in NMP; 0.48 mL). The reaction block (in the case of automated peptide synthesis) or individual reaction vessel (in the case of manual peptide synthesis) was shaken for about 2 min. To the above mixture, diisopropylcarbodiimide (0.5M in NMP; 0.48 mL) was added and the reaction mixture was shaken for 4 h at ambient temperature. Then the reaction block or the individual reaction vessel was purged of reactants by application of a positive pressure of dry nitrogen.

### H. Washing Procedure

Each well of the reaction block was filled with 1.2 mL of NMP and the block was shaken for 5 min. The solution was drained under positive pressure of nitrogen. This procedure was repeated three times. The same procedure was used, with an appropriate volume of NMP, in the case of manual synthesis using individual vessels.

### I. Removal of Fmoc Protecting Group

The resin bearing the Fmoc-protected amino acid was treated with 1.5 mL of 20% piperidine in DMF (v/v) and the reaction block or individual manual synthesis vessel was shaken for 15 min. The solution was drained from the resin. This procedure was repeated once and the resin was washed employing the washing procedure described above.

### J. Final Coupling of Ligand

### DOTA and CMDOTA

The N-terminal amino group of the resin-bound peptide linker construct was deblocked and the resin was washed. A 0.25M solution of the desired ligand and HBTU in NMP was made, and was treated with a two-fold equivalency of DIEA. The resulting solution of activated ligand was added to the resin (1.972 mL; 0.48 mmol) and the reaction mixture was shaken at ambient temperature for 24-30 h. The solution was drained and the resin was washed. The final wash of the resin was conducted with 1.5 mL dichloromethane (3C).

### K. Deprotection and Purification of the Final Peptide

A solution of Reagent B (2 mL; 88:5:5:2—TFA:phenol:water:TIPS) was added to the resin and the reaction block or individual vessel was shaken for 4.5 h at ambient temperature. The resulting solution containing the deprotected peptide was drained into a vial. This procedure was repeated two more times with 1 mL of Reagent B. The combined filtrate was concentrated under reduced pressure using a Genevac HT-12 series II centrifugal concentrator. The residue in each vial was then triturated with 2 mL of Et₂O and the supernatant was decanted. This procedure was repeated twice to provide the peptides as colorless solids. The crude peptides were dissolved in water/acetonitrile and purified using either a Waters XTerra MS C18 preparative HPLC column (50 mm×19 mm, 5 micron particle size, 120A pore size) or a Waters-YMC C18 ODS column (250 mm×30 mm i.d., 10 micron particle size. 120 Å pore size). The product-containing fractions were collected and analyzed by HPLC. The fractions with >95% purity were pooled and the peptides isolated by lyophilization.

Conditions for Preparative HPLC (Waters XTerra Column):

Elution rate: 50 mL/min

### Detection: UV, λ=220 nm

Eluent A: 0.1% aq. TFA; Eluent B: Acetonitrile (0.1% TFA).

Conditions for HPLC Analysis:

Column: Waters XTerra (Waters Co.; 4.6×50 mm; MS C18; 5 micron particle, 120 Å pore).
Elution rate: 3 mL/min; Detection: UV, λ=220 nm.
Eluent A:0.1% aq. TFA; Eluent B: Acetonitrile (0.1% TFA).

### Example 1

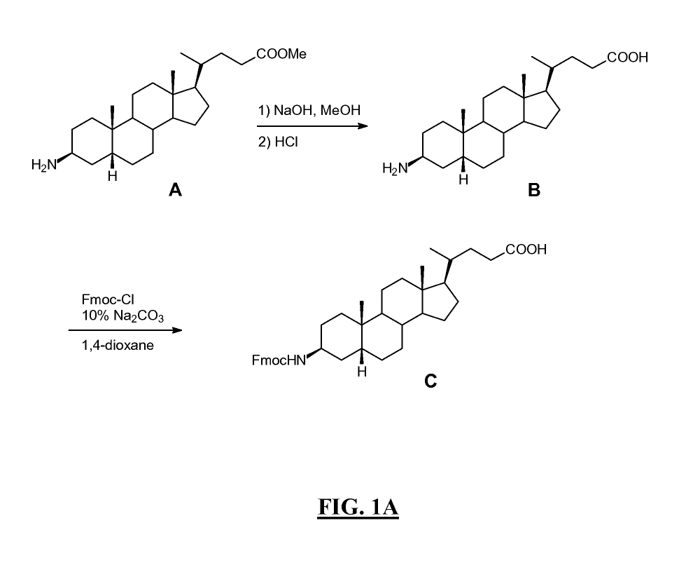

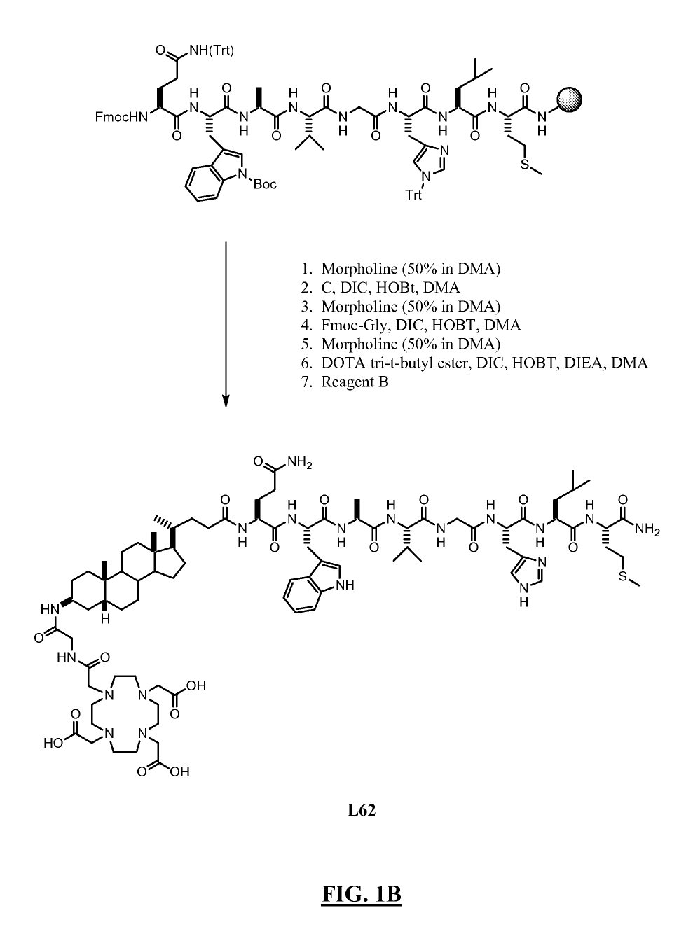

### FIGS. 1A-B

### Synthesis of L62

Summary: As shown in FIGS. 1A-B, L62 was prepared using the following steps: Hydrolysis of (3β, 5β)-3-aminocholan-24-oic acid methyl ester A with NaOH gave the corresponding acid B, which was then reacted with Fmoc-Cl to give intermediate C. Rink amide resin functionalised with the octapeptide Gln-Trp-Ala-Val-Gly-His-Leu-Met-NH₂ (BBN[7-14](SEQ ID NO: 1) was sequentially reacted with C, Fmoc-glycine and DOTA tri-t-butyl ester. After cleavage and deprotection with Reagent B the crude product was purified by preparative HPLC to give L62. Overall yield: 2.5%. More details are provided below:

### A. Rink Amide Resin Functionalised with Bombesin[7-14](SEQ ID NO: 1), (A)

In a solid phase peptide synthesis vessel Fmoc-aminoacid (24 mmol), N-hydroxybenzotriazole (HOBt) (3.67 g; 24 mmol), and N,N′-diisopropylcarbodiimide (DIC) (3.75 mL; 24 mmol) were added sequentially to a suspension of Rink amide NovaGel™ resin (10 g; 6.0 mmol) A in DMF (45 mL). The mixture was shaken for 3 h at room temperature using a bench top shaker, then the solution was emptied and the resin was washed with DMF (5×45 mL). The resin was shaken with 25% piperidine in DMF (45 mL) for 4 min, the solution was emptied and fresh 25% piperidine in DMF (45 mL) was added. The suspension was shaken for 10 min, then the solution was emptied and the resin was washed with DMF (5×45 mL).

This procedure was applied sequentially for the following amino acids: N-α-Fmoc-L-methionine, N-α-Fmoc-L-leucine, N-aC-Fmoc-N^{im}-trityl-L-histidine, N-α-Fmoc-glycine, N-α-Fmoc-L-valine, N-α-Fmoc-L-alanine, N-α-Fmoc-Nⁱⁿ-Boc-L-tryptophan.

In the last coupling reaction N-α-Fmoc-N-γ-trityl-L-glutamine (14.6 g; 24 mmol), HOBt (3.67 g; 24 mmol), and DIC (3.75 mL; 24 mmol) were added to the resin in DMF (45 mL). The mixture was shaken for 3 h at room temperature, the solution was emptied and the resin was washed with DMF (5×45 mL), CH₂Cl₂ (5×45 mL) and vacuum dried.

### B. Preparation of Intermediates B and C

### FIG. 1A

### 1. Synthesis of (3β,5β)-3-Aminocholan-24-oic Acid (B)

A 1 M solution of NaOH (16.6 mL; 16.6 mmol) was added dropwise to a solution of (33,53)-3-aminocholan-24-oic acid methyl ester (5.0 g; 12.8 mmol) in MeOH (65 mL) at 45° C. After 3 h stirring at 45° C., the mixture was concentrated to 25 mL and H₂O (40 mL) and 1 M HCl (22 mL) were added. The precipitated solid was filtered, washed with H₂O (2×50 mL) and vacuum dried to give B as a white solid (5.0 g; 13.3 mmol). Yield 80%.

### 2. Synthesis of (3β,5β)-3-(9H-Fluoren-9-ylmethoxy)aminocholan-24-oic acid (C)

A solution of 9-fluorenylmethoxycarbonyl chloride (0.76 g; 2.93 mmol) in 1,4-dioxane (9 mL) was added dropwise to a suspension of (3β,5β)-3-aminocholan-24-oic acid B (1.0 g; 2.66 mmol) in 10% aq. Na₂CO₃ (16 mL) and 1,4-dioxane (9 mL) stirred at 0° C. After 6 h stirring at room temperature H₂O (90 mL) was added, the aqueous phase washed with Et₂O (2×90 mL) and then 2 M HCl (15 mL) was added (final pH: 1.5). The aqueous phase was extracted with EtOAc (2×100 mL), the organic phase dried over Na₂SO₄ and evaporated. The crude was purified by flash chromatography to give C as a white solid (1.2 g; 2.0 mmol). Yield 69%.

### C. Synthesis of L62 (N-[(3β,5β)-3-[[[[[4,7,10-Tris(carboxymethyl)-1,4,7,10-tetraazacyclododec-1-yl]acetyl]amino]acetyl]amino]-cholan-24-yl]-L-glutaminyl-L-tryptophyl-L-alanyl-L-valyl-glycyl-L-histidyl-L-leucyl-L-methioninamide) (FIG. 1B)

Resin A (0.5 g; 0.3 mmol) was shaken in a solid phase peptide synthesis vessel with 50% morpholine in DMA (7 mL) for 10 min, the solution was emptied and fresh 50% morpholine in DMA (7 mL) was added. The suspension was shaken for 20 min then the solution was emptied and the resin washed with DMA (5×7 mL). (3β,5β)-3-(9H-Fluoren-9-ylmethoxy)aminocholan-24-oic acid C (0.72 g; 1.2 mmol), N-hydroxybenzotriazole (HOBt) (0.18 g; 1.2 mmol), N,N′-diisopropylcarbodiimide (DIC) (0.19 mL; 1.2 mmol) and DMA (7 mL) were added to the resin, the mixture shaken for 24 h at room temperature, and the solution was emptied and the resin washed with DMA (5×7 mL). The resin was then shaken with 50% morpholine in DMA (7 mL) for 10 min, the solution was emptied, fresh 50% morpholine in DMA (7 mL) was added and the mixture shaken for another 20 min. The solution was emptied and the resin washed with DMA (5×7 mL). N-α-Fmoc-glycine (0.79 g; 1.2 mmol), HOBt (0.18 g; 1.2 mmol), DIC (0.19 mL: 1.2 mmol) and DMA (7 mL) were added to the resin. The mixture was shaken for 3 h at room temperature, the solution was emptied and the resin washed with DMA (5×7 mL). The resin was then shaken with 50% morpholine in DMA (7 mL) for 10 min, the solution was emptied, fresh 50% morpholine in DMA (7 mL) was added and the mixture shaken for another 20 min. The solution was emptied and the resin washed with DMA (5×7 mL) followed by addition of 1,4,7,10-tetraazacyclododecane-1,4,7,10-tetraacetic acid tris(1,1-dimethylethyl) ester adduct with NaCl (0.79 g; 1.2 mmol), HOBt (0.18 g; 1.2 mmol), DIC (0.19 mL: 1.2 mmol), DIEA (0.40 mL; 2.4 mmol) and DMA (7 mL) to the resin. The mixture was shaken for 24 h at room temperature, the solution was emptied and the resin washed with DMA (5×7 mL), CH₂Cl₂ (5×7 mL) and vacuum dried. The resin was shaken in a flask with Reagent B (25 mL) for 4.5 h. The resin was filtered and the solution was evaporated under reduced pressure to afford an oily crude which was triturated with Et₂O (20 mL) gave a precipitate. The precipitate was collected by centrifugation and washed with Et₂O (3×20 mL), then analysed by HPLC and purified by preparative HPLC. The fractions containing the product were lyophilised to give L62 (6.6 mg; 3.8×10⁻³ mmol) as a white solid. Yield 4.5%.

### Example II

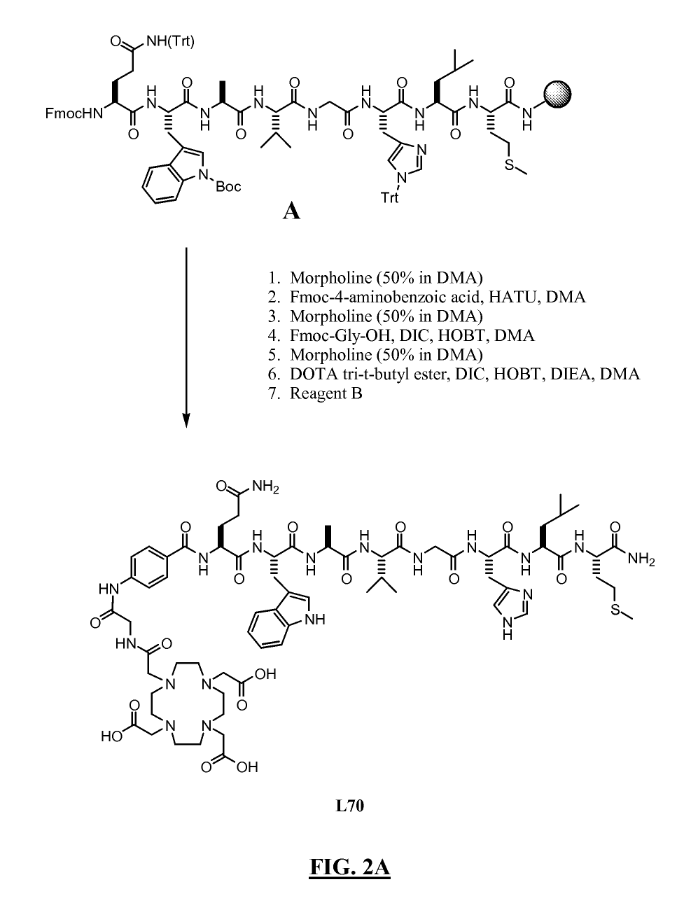

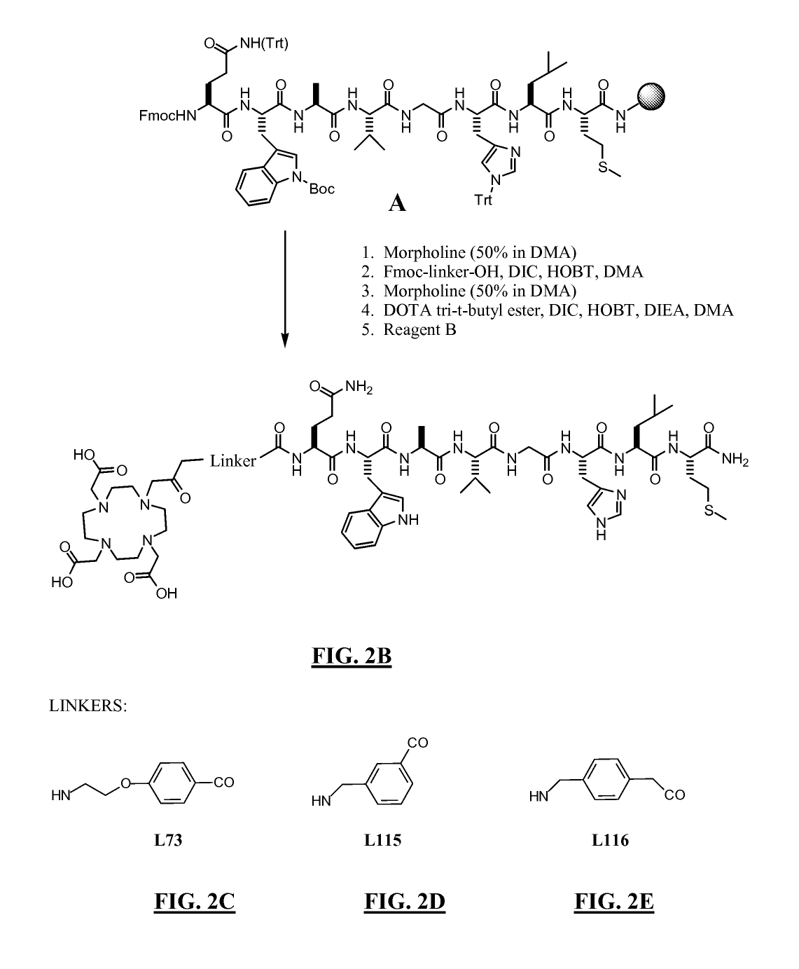

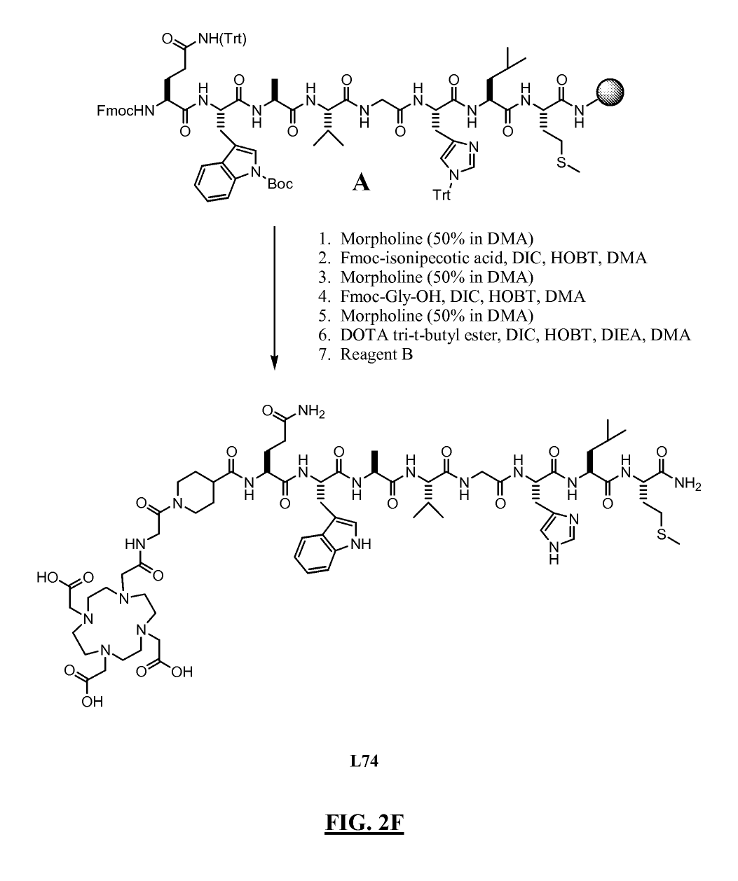

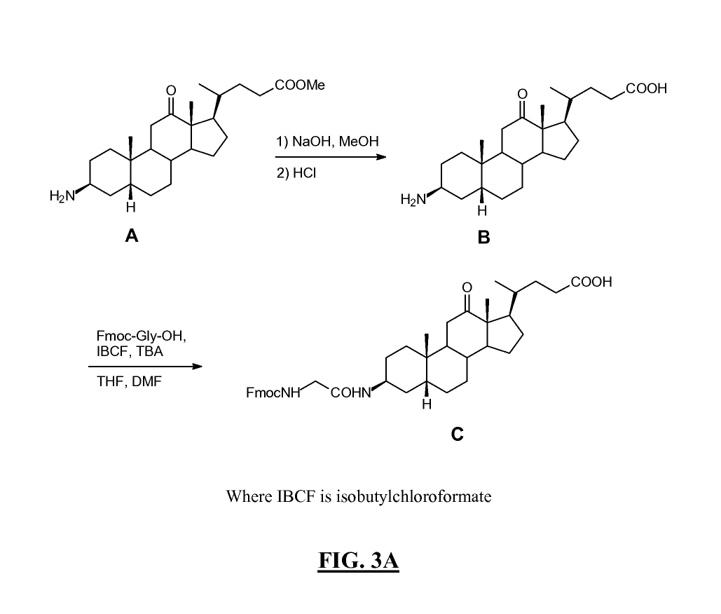

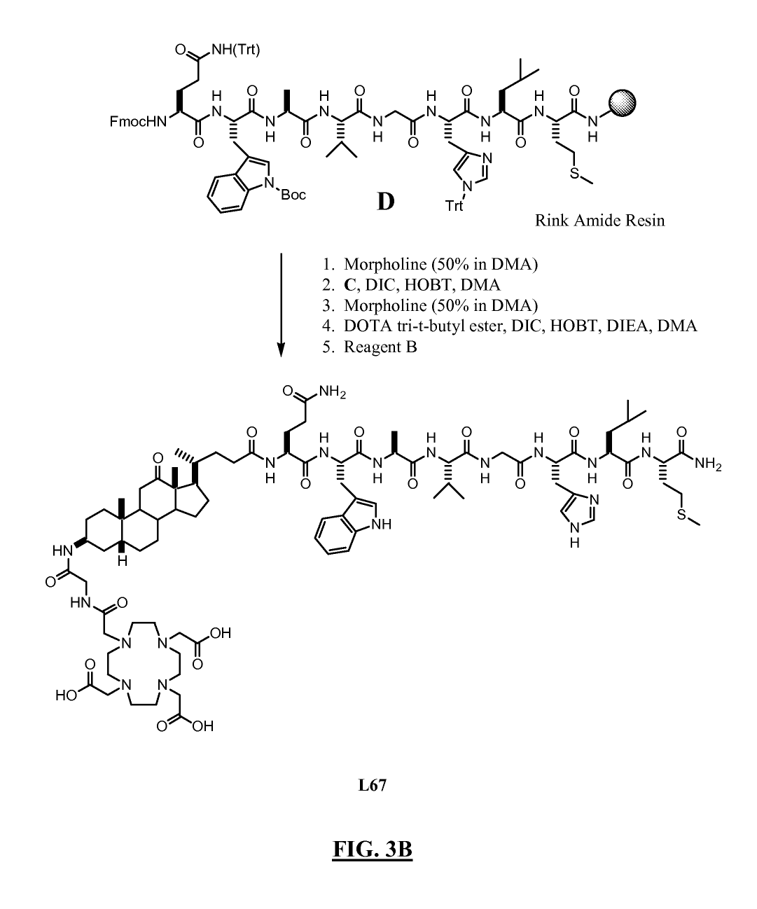

### FIGS. 2A-F

### Synthesis of L70, L73, L74, L115 and L116

Summary: The products were obtained by coupling of the octapeptide Gln-Trp-Ala-Val-Gly-His-Leu-Met-NH₂ (BBN(7-14) (SEQ ID NO:1) (with appropriate side chain protection) on the Rink amide resin with different linkers, followed by functionalization with DOTA tri-t-butyl ester. After cleavage and deprotection with Reagent B the final products were purified by preparative HPLC. Overall yields 3-9%.

A. Synthesis of L70 (FIG. 2A):

Resin A (0.5 g; 0.3 mmol) was shaken in a solid phase peptide synthesis vessel with 50% morpholine in DMA (7 mL) for 10 min, the solution was emptied and fresh 50% morpholine in DMA (7 mL) was added. The suspension was stirred for 20 min then the solution was emptied and the resin washed with DMA (5×7 mL). Fmoc-4-aminobenzoic acid (0.43 g; 1.2 mmol), HOBt (0.18 g; 1.2 mmol), DIC (0.19 mL; 1.2 mmol) and DMA (7 mL) were added to the resin, the mixture shaken for 3 h at room temperature, the solution was emptied and the resin washed with DMA (5×7 mL). The resin was then shaken with 50% morpholine in DMA (7 mL) for 10 min, the solution was emptied, fresh 50% morpholine in DMA (7 mL) was added and the mixture was shaken for 20 min. The solution was emptied and the resin washed with DMA (5×7 mL). Fmoc-glycine (0.36 g; 1.2 mmol) HATU (0.46 g; 1.2 mmol) and DIEA (0.40 mL; 2.4 mmol) were stirred for 15 min in DMA (7 mL) then the solution was added to the resin, the mixture shaken for 2 h at room temperature, the solution was emptied and the resin washed with DMA (5×7 mL). The resin was then shaken with 50% morpholine in DMA (7 mL) for 10 min, the solution was emptied, fresh 50% morpholine in DMA (7 mL) was added and the mixture shaken for 20 min. The solution was emptied and the resin washed with DMA (5×7 mL). 1,4,7,10-Tetraazacyclododecane-1,4,7,10-tetraacetic acid tris(1,1-dimethylethyl) ester adduct with NaCl (0.79 g; 1.2 mmol), HOBt (0.18 g; 1.2 mmol), DIC (0.19 mL: 1.2 mmol), DIEA (0.40 mL; 2.4 mmol) and DMA (7 mL) were added to the resin. The mixture was shaken for 24 h at room temperature, the solution was emptied and the resin washed with DMA (5×7 mL), CH₂Cl₂ (5×7 mL) and vacuum dried. The resin was shaken in a flask with Reagent B (25 mL) for 4 h. The resin was filtered and the filtrate solution was evaporated under reduced pressure to afford an oily crude that was triturated with Et₂O (5 mL). The precipitate was collected by centrifugation and washed with Et₂O (5×5 mL), then analysed by HPLC and purified by preparative HPLC. The fractions containing the product were lyophilised to give L70 as a white fluffy solid (6.8 mg; 0.005 mmol). Yield 3%.

B. Synthesis of L73, L115 and L116 (FIGS. 2B-2E):

Resin A (0.5 g; 0.3 mmol) was shaken in a solid phase peptide synthesis vessel with 50% morpholine in DMA (7 mL) for 10 min, the solution was emptied and fresh 50% morpholine in DMA (7 mL) was added. The suspension was stirred for 20 min then the solution was emptied and the resin washed with DMA (5×7 mL). Fmoc-linker-OH (1.2 mmol), HOBt (0.18 g; 1.2 mmol), DIC (0.19 mL; 1.2 mmol) and DMA (7 mL) were added to the resin, the mixture was shaken for 3 h at room temperature, the solution was emptied and the resin was washed with DMA (5×7 mL). The resin was shaken with 50% morpholine in DMA (7 mL) for 10 min, the solution was emptied, fresh 50% morpholine in DMA (7 mL) was added and the mixture was shaken for 20 min. The solution was emptied and the resin washed with DMA (5×7 mL). 1,4,7,10-Tetraazacyclododecane-1,4,7,10-tetraacetic acid tris(1,1-dimethylethyl) ester adduct with NaCl (0.79 g; 1.2 mmol), HOBt (0.18 g; 1.2 mmol), DIC (0.19 mL: 1.2 mmol), DIEA (0.40 mL; 2.4 mmol) and DMA (7 mL) were added to the resin. The mixture was shaken for 24 h at room temperature, the solution was emptied and the resin washed with DMA (5×7 mL), CH₂Cl₂ (5×7 mL) and vacuum dried. The resin was shaken in a flask with Reagent B (25 mL) for 4 h. The resin was filtered and the solution was evaporated under reduced pressure to afford an oily crude that was triturated with Et₂O (5 mL). The precipitate was collected by centrifugation and washed with Et₂O (5×5 mL), then analysed by HPLC and purified by preparative HPLC. The fractions containing the product were lyophilised.

C. Synthesis of L74 (FIG. 2F)

Resin A (0.5 g; 0.3 mmol) was shaken in a solid phase peptide synthesis vessel with 50% morpholine in DMA (7 mL) for 10 min, the solution was emptied and fresh 50% morpholine in DMA (7 mL) was added. The suspension was stirred for 20 min then the solution was emptied and the resin was washed with DMA (5×7 mL). Fmoc-isonipecotic acid (0.42 g; 1.2 mmol), HOBt (0.18 g; 1.2 mmol), DIC (0.19 mL; 1.2 mmol) and DMA (7 mL) were added to the resin, the mixture was shaken for 3 h at room temperature, the solution was emptied and the resin was washed with DMA (5×7 mL). The resin was shaken with 50% morpholine in DMA (7 mL) for 10 min, the solution was emptied, fresh 50% morpholine in DMA (7 mL) was added and the mixture was shaken for 20 min. The solution was emptied and the resin was washed with DMA (5×7 mL). Fmoc-glycine (0.36 g; 1.2 mmol), HOBt (0.18 g; 1.2 mmol), DIC (0.19 mL; 1.2 mmol) and DMA (7 mL) were added to the resin, the mixture was shaken for 3 h at room temperature, the solution was emptied and the resin washed with DMA (5×7 mL). The resin was then shaken with 50% morpholine in DMA (7 mL) for 10 min, the solution was emptied, fresh 50% morpholine in DMA (7 mL) was added and the mixture shaken for 20 minutes. The solution was emptied and the resin was washed with DMA (5×7 mL). 1,4,7,10-Tetraazacyclododecane-1,4,7,10-tetraacetic acid tris(1,1-dimethylethyl) ester adduct with NaCl (0.79 g; 1.2 mmol), HOBt (0.18 g; 1.2 mmol), DIC (0.19 mL: 1.2 mmol), DIEA (0.40 mL; 2.4 mmol) and DMA (7 mL) were added to the resin. The mixture was shaken for 24 h at room temperature, the solution was emptied and the resin was washed with DMA (5×7 mL), CH₂Cl₂ (5×7 mL) and vacuum dried. The resin was shaken in a flask with Reagent B (25 mL) for 4 h. The resin was filtered and the solution was evaporated under reduced pressure to afford an oily crude that was triturated with Et₂O (5 mL). The precipitate was collected by centrifugation and washed with Et₂O (5×5 mL), then analysed by HPLC and purified by HPPLC. The fractions containing the product were lyophilised to give L74 as a white fluffy solid (18.0 mg; 0.012 mmol). Yield 8%.

### Example III

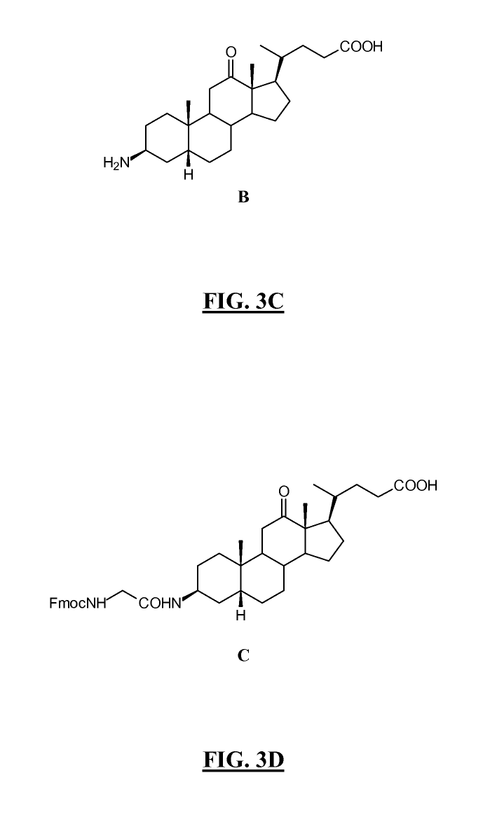

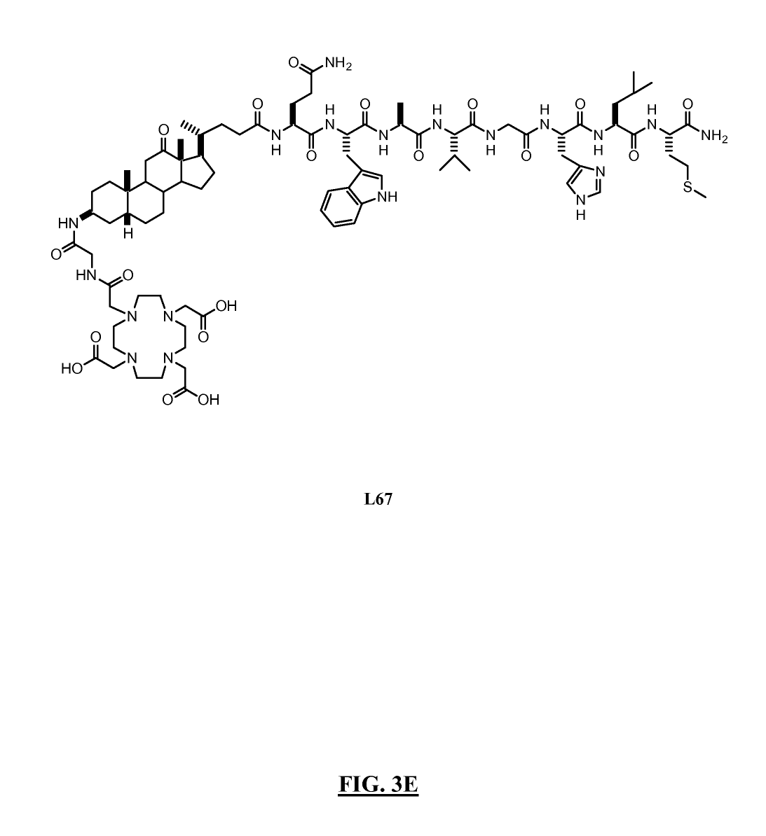

### FIGS. 3A-E

### Synthesis of L67

Summary: Hydrolysis of (3β,5β)-3-amino-12-oxocholan-24-oic acid methyl ester A with NaOH gave the corresponding acid B, which was then reacted with Fmoc-Glycine to give intermediate C. Rink amide resin functionalised with the octapeptide Gln-Trp-Ala-Val-Gly-His-Leu-Met-NH₂ (BBN[7-14](SEQ ID NO: 1) was sequentially reacted with C, and DOTA tri-t-butyl ester. After cleavage and deprotection with Reagent B the crude was purified by preparative HPLC to give L67. Overall yield: 5.2%.

### A. Synthesis (3β,5β)-3-Amino-12-oxocholan-24-oic acid, (B) (FIG. 3A)

A 1 M solution of NaOH (6.6 mL; 6.6 mmol) was added dropwise to a solution of (3β,5β)-3-amino-12-oxocholan-24-oic acid methyl ester A (2.1 g; 5.1 mmol) in MeOH (15 mL) at 45° C. After 3 h stirring at 45° C., the mixture was concentrated to 25 mL then H₂O (25 mL) and 1 M HCl (8 mL) were added. The precipitated solid was filtered, washed with H₂O (2×30 mL) and vacuum dried to give B as a white solid (1.7 g; 4.4 mmol). Yield 88%.

### B. Synthesis of (313,513)-3-[[(9H-Fluoren-9-ylmethoxy)amino]acetyl]amino-12-oxocholan-24-oic acid (C) (FIG. 3A)

Tributylamine (0.7 mL; 3.1 mmol) was added dropwise to a solution of N-α-Fmoc-glycine (0.9 g; 3.1 mmol) in THF (25 mL) stirred at 0° C. Isobutyl chloroformate (0.4 mL; 3.1 mmol) was subsequently added and, after 10 min, a suspension of tributylamine (0.6 mL; 2.6 mmol) and (3β,5β)-3-amino-12-oxocholan-24-oic acid B (1.0 g; 2.6 mmol) in DMF (30 mL) was added dropwise, over 1 h, into the cooled solution. The mixture was allowed to warm up and after 6 h the solution was concentrated to 40 mL, then H₂O (50 mL) and 1 N HCl (10 mL) were added (final pH: 1.5). The precipitated solid was filtered, washed with H₂O (2×50 mL), vacuum dried and purified by flash chromatography to give C as a white solid (1.1 g; 1.7 mmol). Yield 66%.

### C. Synthesis of L67 (N-[(313,513)-3-[[[[[4,7,10-Tris(carboxymethyl)-1,4,7,10-tetraazacyclododec-1-yl]acetyl]amino]acetyl]amino]-12,24-dioxocholan-24-yl]-L-glutaminyl-L-tryptophyl-L-alanyl-L-valyl-glycyl-L-histidyl-L-leucyl-L-methioninamide) (FIG. 3B and FIG. 3E)

Resin D (0.5 g; 0.3 mmol) was shaken in a solid phase peptide synthesis vessel with 50% morpholine in DMA (7 mL) for 10 min, the solution was emptied and fresh 50% morpholine in DMA (7 mL) was added. The suspension was stirred for 20 min then the solution was emptied and the resin was washed with DMA (5×7 mL). (3β,5β)-3-[[(9H-Fluoren-9-ylmethoxy)amino]acetyl]amino]-12-oxocholan-24-oic acid C (0.80 g; 1.2 mmol), HOBt (0.18 g; 1.2 mmol), DIC (0.19 mL; 1.2 mmol) and DMA (7 mL) were added to the resin, the mixture was shaken for 24 h at room temperature, the solution was emptied and the resin was washed with DMA (5×7 mL). The resin was shaken with 50% morpholine in DMA (7 mL) for 10 min, the solution was emptied, fresh 50% morpholine in DMA (7 mL) was added and the mixture was shaken for 20 min. The solution was emptied and the resin was washed with DMA (5×7 mL). 1,4,7,10-Tetraazacyclododecane-1,4,7,10-tetraacetic acid tris(1,1-dimethylethyl) ester adduct with NaCl (0.79 g; 1.2 mmol), HOBt (0.18 g; 1.2 mmol), DIC (0.19 mL: 1.2 mmol), DIEA (0.40 mL; 2.4 mmol) and DMA (7 mL) were added to the resin. The mixture was shaken for 24 h at room temperature, the solution was emptied and the resin was washed with DMA (5×7 mL), CH₂Cl₂ (5×7 mL) and vacuum dried. The resin was shaken in a flask with Reagent B (25 mL) for 4.5 h. The resin was filtered and the solution was evaporated under reduced pressure to afford an oily crude that was triturated with Et₂O (20 mL).

### Example IV

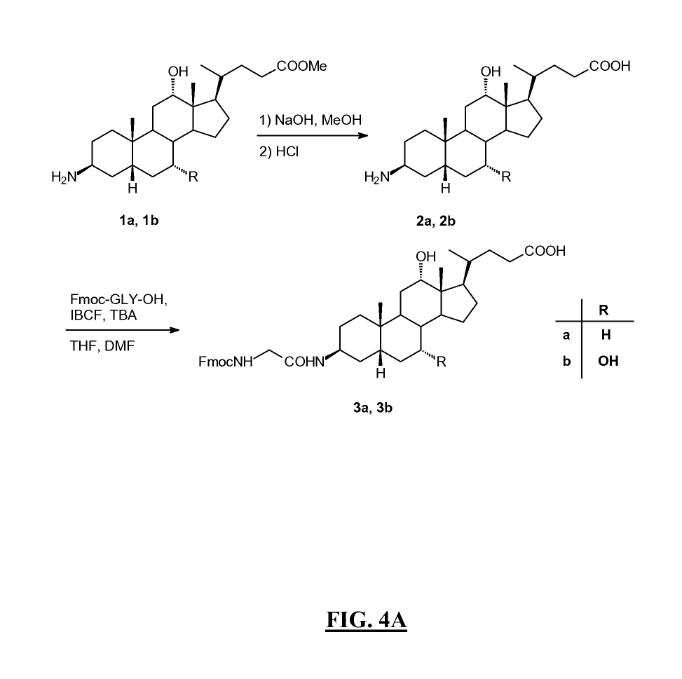

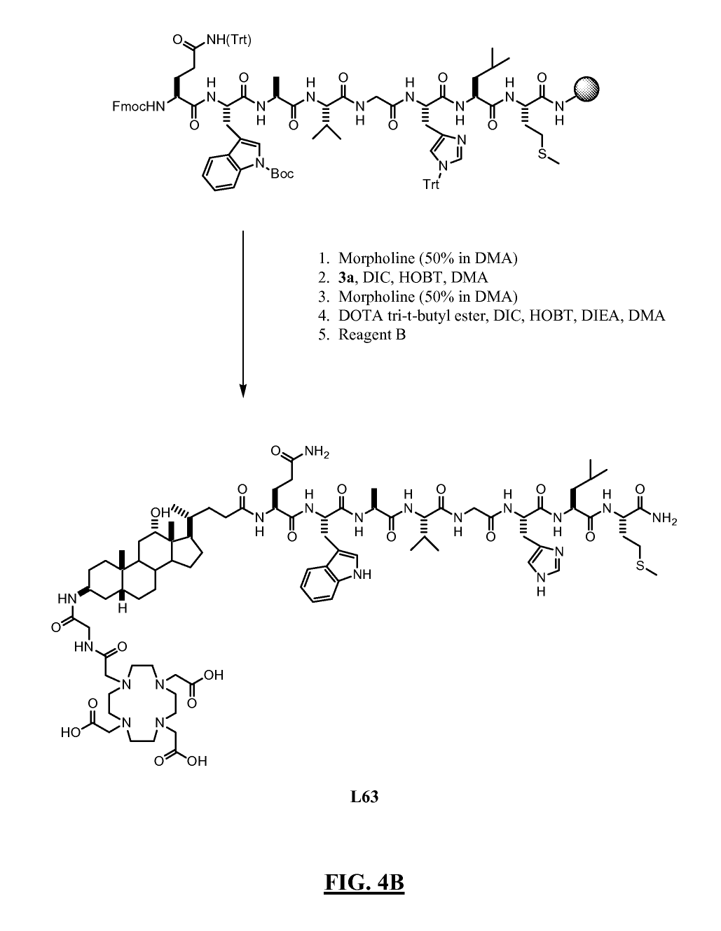

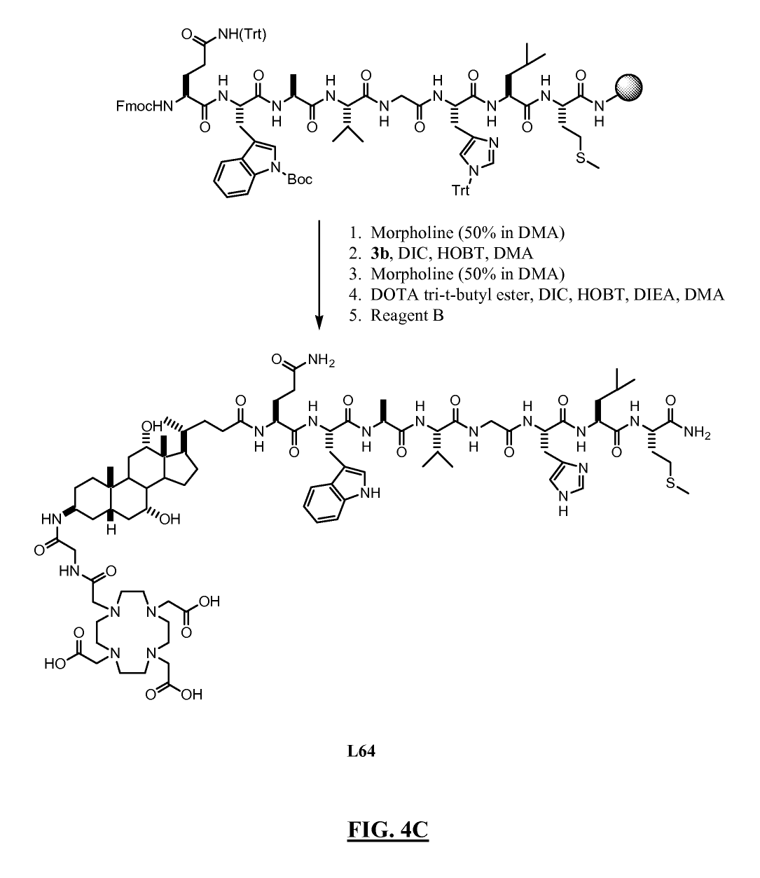

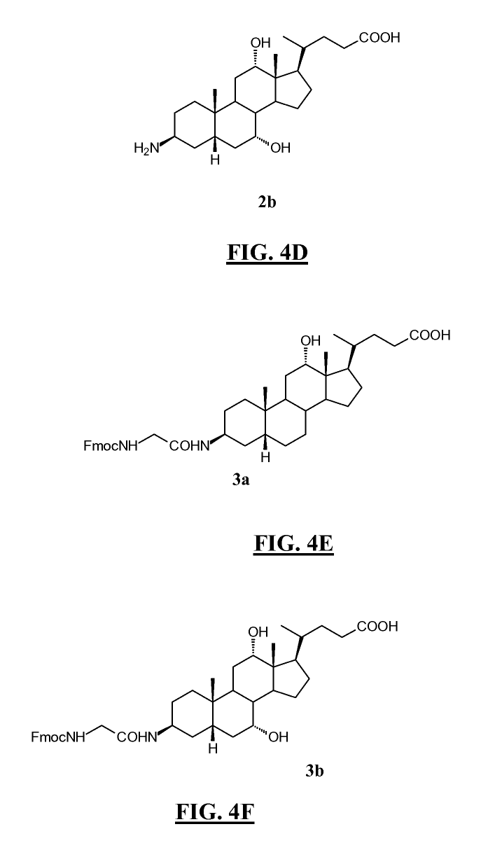

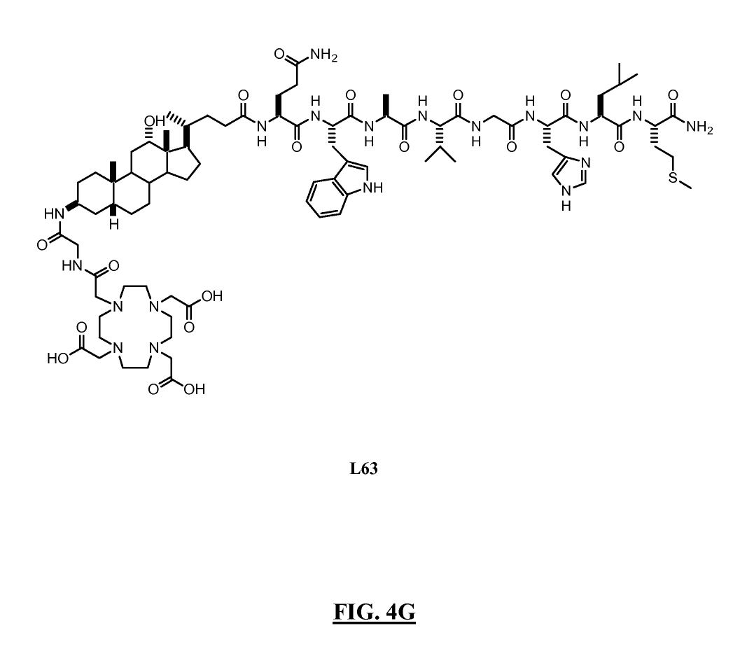

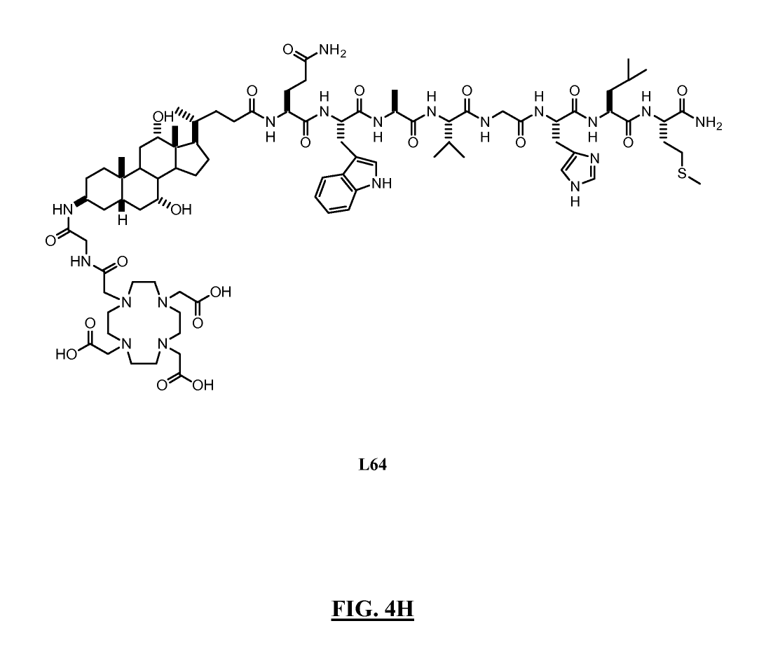

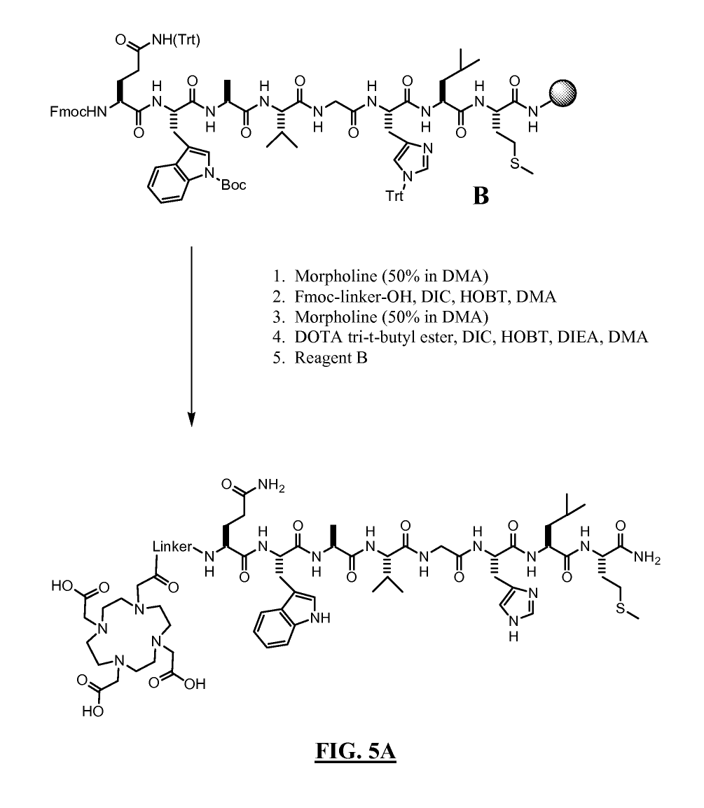

### FIGS. 4A-H

### Synthesis of L63 and L64

Summary: Hydrolysis of (3β,5β,7α,12α)-3-amino-7,12-dihydroxycholan-24-oic acid methyl ester 1b with NaOH gave the intermediate 2b, which was then reacted with Fmoc-glycine to give 3b. Rink amide resin functionalised with the octapeptide Gln-Trp-Ala-Val-Gly-His-Leu-Met-NH₂ (BBN[7-14](SEQ ID NO:1) was reacted with 3b and then with DOTA tri-t-butyl ester. After cleavage and deprotection with Reagent B the crude was purified by preparative HPLC to give L64. The same procedure was repeated starting from intermediate 2a, already available, to give L63. Overall yields: 9 and 4%, respectively.

### A. Synthesis of (3β,5β,7α,12α)-3-Amino-7,12-dihydroxycholan-24-oic acid, (2b) (FIG. 4A)

A 1 M solution of NaOH (130 mL; 0.13 mol) was added dropwise to a solution of (3β,5β,7α,12α)-3-amino-7,12-dihydroxycholan-24-oic acid methyl ester 1b (42.1 g; 0.10 mol) in MeOH (300 mL) heated at 45° C. After 3 h stirring at 45° C., the mixture was concentrated to 150 mL and H₂O (350 mL) was added. After extraction with CH₂Cl₂ (2×100 mL) the aqueous solution was concentrated to 200 mL and 1 M HCl (150 mL) was added. The precipitated solid was filtered, washed with H₂O (2×100 mL) and vacuum dried to give 2b as a white solid (34.8 g; 0.08 mol). Yield 80%.

### B. Synthesis of (3β,5β,12α)-3-[[(9H-Fluoren-9-ylmethoxy)amino]acetyl]amino-12-hydroxycholan-24-oic acid, (3a) (FIG. 4A)

Tributylamine (4.8 mL; 20.2 mmol) was added dropwise to a solution of N-α-Fmoc-glycine (6.0 g; 20.2 mmol) in THF (120 mL) stirred at 0° C. Isobutyl chloroformate (2.6 mL; 20.2 mmol) was subsequently added and, after 10 min, a suspension of tributylamine (3.9 mL; 16.8 mmol) and (3P,5J, 12a)-3-amino-12-hydroxycholan-24-oic acid 2a (6.6 g; 16.8 mmol) in DMF (120 mL) was added dropwise, over 1 h, into the cooled solution. The mixture was allowed to warm up and after 6 h the solution was concentrated to 150 mL, then H₂O (250 mL) and 1 N HCl (40 mL) were added (final pH: 1.5). The precipitated solid was filtered, washed with H₂O (2×100 mL), vacuum dried and purified by flash chromatography to give 3a as a white solid (3.5 g; 5.2 mmol). Yield 31%.

### C. Synthesis of (3β,5β,7α,12α)-3-[[(9H-Fluoren-9-ylmethoxy)amino]acetyl]amino-7,12-dihydroxycholan-24-oic acid, (3b) (FIG. 4A)

Tributylamine (3.2 mL; 13.5 mmol) was added dropwise to a solution of N-α-Fmoc-glycine (4.0 g; 13.5 mmol) in THF (80 mL) stirred at 0° C. Isobutyl chloroformate (1.7 mL; 13.5 mmol) was subsequently added and, after 10 min, a suspension of tributylamine (2.6 mL; 11.2 mmol) and (3β,5β,7α,12α)-3-amino-7,12-dihydroxycholan-24-oic acid 3a (4.5 g; 11.2 mmol) in DMF (80 mL) was added dropwise, over 1 h, into the cooled solution. The mixture was allowed to warm up and after 6 h the solution was concentrated to 120 mL, then H₂O (180 mL) and 1 N HCl (30 mL) were added (final pH: 1.5). The precipitated solid was filtered, washed with H₂O (2×100 mL), vacuum dried and purified by flash chromatography to give 3a as a white solid (1.9 g; 2.8 mmol). Yield 25%.

In an alternative method, (3β,5β,7α,12α)-3-[[(9H-Fluoren-9-ylmethoxy)amino]acetyl]amino-7,12-dihydroxycholan-24-oic acid, (3b) can be prepared as follows:

N-Hydroxysuccinimide (1.70 g, 14.77 mmol) and DIC (1.87 g, 14.77 mmol) were added sequentially to a stirred solution of Fmoc-Gly-OH (4.0 g, 13.45 mmol) in dichloromethane (15 mL); the resulting mixture was stirred at room temperature for 4 h. The N,N′-diisopropylurea formed was removed by filtration and the solid was washed with ether (20 mL). The volatiles were removed and the solid Fmoc-Gly-succinimidyl ester formed was washed with ether (3×20 mL). Fmoc-Gly-succinimidyl ester was then redissolved in dry DMF (15 mL) and 3-aminodeoxycholic acid (5.21 g, 12.78 mmol) was added to the clear solution. The reaction mixture was stirred at room temperature for 4 h, water (200 mL) was added and the precipitated solid was filtered, washed with water, dried and purified by silica gel chromatography (TLC (silica): (R_{f}: 0.50, silica gel, CH₂Cl₂/CH₃OH, 9:1) (eluant: CH₂Cl₂/CH₃OH (9:1)) to give (3β,5β,7α,12α)-3-[[(9H-Fluoren-9-ylmethoxy)amino]acetyl]amino-7,12-dihydroxycholan-24-oic acid as a colorless solid. Yield: 7.46 g (85%).

### D. Synthesis of L63 (N-[(3β,5β,12α)-3-[[[[[4,7,10-Tris(carboxymethyl)-1,4,7,10-tetraazacyclododec-1-yl]acetyl]amino]acetyl]amino]-12-hydroxy-24-oxocholan-24-yl]-L-glutaminyl-L-tryptophyl-L-alanyl-L-valyl-glycyl-L-histidvl-L-leucvl-L-methioninamide) (FIG. 4B)

Resin A (0.5 g; 0.3 mmol) was shaken in a solid phase peptide synthesis vessel with 50% morpholine in DMA (7 mL) for 10 min, the solution was emptied and fresh 50% morpholine in DMA (7 mL) was added. The suspension was stirred for 20 min then the solution was emptied and the resin washed with DMA (5×7 mL). (3β,5β,12α)-3-[[(9H-Fluoren-9-ylmethoxy)amino]acetyl]amino-12-hydroxycholan-24-oic acid 3a (0.82 g; 1.2 mmol), HOBt (0.18 g; 1.2 mmol), DIC (0.19 mL; 1.2 mmol) and DMA (7 mL) were added to the resin, the mixture was shaken for 24 h at room temperature, the solution was emptied and the resin was washed with DMA (5×7 mL). The resin was then shaken with 50% morpholine in DMA (7 mL) for 10 min, the solution was emptied, fresh 50% morpholine in DMA (7 mL) was added and the mixture was shaken for 20 min. The solution was emptied and the resin washed with DMA (5×7 mL). 1,4,7,10-Tetraazacyclododecane-1,4,7,10-tetraacetic acid tris(1,1-dimethylethyl) ester adduct with NaCl (0.79 g; 1.2 mmol), HOBt (0.18 g; 1.2 mmol), DIC (0.19 mL: 1.2 mmol), DIEA (0.40 mL; 2.4 mmol) and DMA (7 mL) were added to the resin. The mixture was shaken for 24 h at room temperature, the solution was emptied and the resin washed with DMA (5×7 mL), CH₂Cl₂ (5×7 mL) and vacuum dried. The resin was shaken in a flask with Reagent B (25 mL) for 4 h. The resin was filtered and the solution was evaporated under reduced pressure to afford an oily crude that after treatment with Et₂O (5 mL) gave a precipitate. The precipitate was collected by centrifugation and washed with Et₂O (5×5 mL), then analysed and purified by HPLC. The fractions containing the product were lyophilised to give L63 as a white fluffy solid (12.8 mg; 0.0073 mmol). Yield 9%.

### E. Synthesis of L64 (N-[(3β,5β,7α,12α)-3-[[[[[4,7,10-Tris(carboxymethyl)-1,4,7,10-tetraazacyclododec-1-yl]acetyl]amino]acetyl]amino]-7,12-dihydroxy-24-oxocholan-24-yl]-L-glutaminyl-L-tryptophyl-L-alanyl-L-valyl-glycyl-L-histidyl-L-leucyl-L-methioninamide) (FIG. 4C)

Resin A (0.5 g; 0.3 mmol) was shaken in a solid phase peptide synthesis vessel with 50% morpholine in DMA (7 mL) for 10 min, the solution was emptied and fresh 50% morpholine in DMA (7 mL) was added. The suspension was stirred for 20 min, the solution was emptied and the resin was washed with DMA (5×7 mL). (3β,5β,7α,12α)-3-[[(9H-Fluoren-9-ylmethoxy)amino]acetyl]amino-7,12-dihydroxycholan-24-oic acid 3b (0.81 g; 1.2 mmol), HOBt (0.18 g; 1.2 mmol), DIC (0.19 mL; 1.2 mmol) and DMA (7 mL) were added to the resin, the mixture was shaken for 24 h at room temperature, the solution was emptied and the resin was washed with DMA (5×7 mL). The resin was shaken with 50% morpholine in DMA (7 mL) for 10 min, the solution was emptied, fresh 50% morpholine in DMA (7 mL) was added and the mixture was shaken for 20 min. The solution was emptied and the resin was washed with DMA (5×7 mL). 1,4,7,10-Tetraazacyclododecane-1,4,7,10-tetraacetic acid tris(1,1-dimethylethyl) ester adduct with NaCl (0.79 g; 1.2 mmol), HOBt (0.18 g; 1.2 mmol), DIC (0.19 mL: 1.2 mmol), DIEA (0.40 mL; 2.4 mmol) and DMA (7 mL) were added to the resin. The mixture was shaken for 24 h at room temperature, the solution was emptied and the resin washed with DMA (5×7 mL), CH₂Cl₂ (5×7 mL) and vacuum dried. The resin was shaken in a flask with Reagent B (25 mL) for 4 h. The resin was filtered and the solution was evaporated under reduced pressure to afford an oily crude that was triturated with Et₂O (5 mL). The precipitate was collected by centrifugation and washed with Et₂O (5×5 mL). Then it was dissolved in H₂O (20 mL), and Na₂CO₃ (0.10 g; 0.70 mmol) was added; the resulting mixture was stirred 4 h at room temperature. This solution was purified by HPLC, the fractions containing the product lyophilised to give L64 as a white fluffy solid (3.6 mg; 0.0021 mmol). Yield 4%.

### Example V

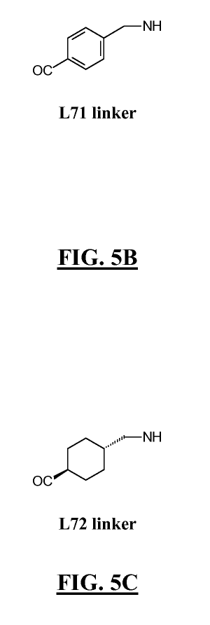

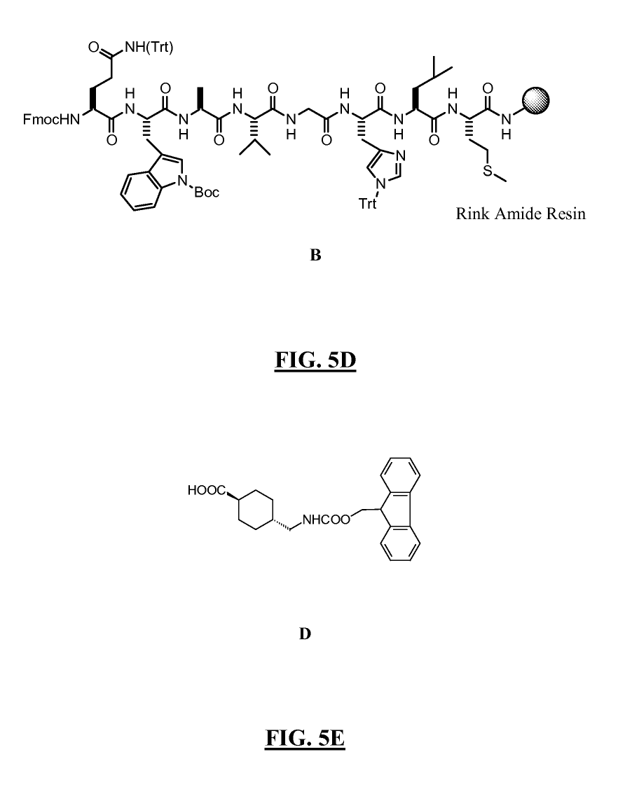

### FIGS. 5A-E

### Synthesis of L71 and L72

Summary: The products were obtained in two steps. The first step was the solid phase synthesis of the octapeptide Gln-Trp-Ala-Val-Gly-His-Leu-Met-NH₂ (BBN[7-14](SEQ ID NO: 1) (with appropriate side chain protecting groups) on the Rink amide resin discussed supra. The second step was the coupling with different linkers followed by functionalization with DOTA tri-t-butyl ester. After cleavage and deprotection with Reagent B the final products were purified by preparative HPLC. Overall yields 3-9%.

### A. Bombesin [7-14]Functionalisation and Cleavage Procedure (FIGS. 5A and 5D)

The resin B (0.5 g; 0.3 mmol) was shaken in a solid phase peptide synthesis vessel with 50% morpholine in DMA (7 mL) for 10 min, the solution was emptied and fresh 50% morpholine in DMA (7 mL) was added. The suspension was stirred for 20 min then the solution was emptied and the resin was washed with DMA (5×7 mL). The Fmoc-linker-OH (1.2 mmol), HOBt (0.18 g; 1.2 mmol), DIC (0.19 mL; 1.2 mmol) and DMA (7 mL) were added to the resin. The mixture was shaken for 3 h at room temperature, the solution was emptied and the resin washed with DMA (5×7 mL). The resin was then shaken with 50% morpholine in DMA (7 mL) for 10 min, the solution was emptied, fresh 50% morpholine in DMA (7 mL) was added and the mixture was shaken for 20 min. The solution was emptied and the resin was washed with DMA (5×7 mL). 1,4,7,10-Tetraazacyclododecane-1,4,7,10-tetraacetic acid tris(1,1-dimethylethyl) ester adduct with NaCl—C (0.79 g; 1.2 mmol), HOBt (0.18 g; 1.2 mmol), DIC (0.19 mL: 1.2 mmol), DIEA (0.40 mL; 2.4 mmol) and DMA (7 mL) were added to the resin. The mixture was shaken for 24 h at room temperature. The solution was emptied and the resin washed with DMA (5×7 mL), CH₂Cl₂ (5×7 mL) and vacuum dried. The resin was shaken in a flask with Reagent B (25 mL) for 4 h. The resin was filtered and the filtrate was evaporated under reduced pressure to afford an oily crude that was triturated with ether (5 mL). The precipitate was collected by centrifugation and washed with ether (5×5 mL), then analyzed by analytical HPLC and purified by preparative HPLC. The fractions containing the product were lyophilized.

### B. Products

1. L71 (4-[[[[4,7,10-Tris(carboxymethyl)-1,4,7,10-tetraazacyclododec-1-yl]acetyl]amino]methyl]benzoyl-L-glutaminyl-L-tryptophyl-L-alanyl-L-valyl-glycyl-L-histidyl-L-leucyl-L-methioninamide)
   The product was obtained as a white fluffy solid (7.3 mg; 0.005 mmol). Yield 7.5%.
2. L72 (Trans-4-[[[[4,7,10-tris(carboxymethyl)-1,4,7,10-tetraazacyclododec-1-yl]acetyl]amino]methyl]cyclohexylcarbonyl-L-glutaminyl-L-tryptophyl-L-alanyl-L-valyl-glycil-L-histidyl-L-leucyl-L-methioninamide)
   The product was obtained as a white fluffy solid (7.0 mg; 0.005 mmol). Yield 4.8%.

### C. Trans-4-[[[(9H-fluoren-9-ylmethoxy)carbonyl]amino]methyl]cyclohexanecarboxylic acid, (D) (FIG. 5E)

A solution of N-(9-fluorenylmethoxycarbonyloxy)succinimide (4.4 g; 14.0 mmol) in 1,4-dioxane (40 mL) was added dropwise to a solution of trans-4-(aminomethyl)cyclohexanecarboxylic acid (2.0 g; 12.7 mmol) in 10% Na₂CO₃ (30 mL) cooled to 0° C. The mixture was then allowed to warm to ambient temperature and after 1 h stirring at room temperature was treated with 1 N HCl (32 mL) until the final pH was 2. The resulting solution was extracted with n-BuOH (100 mL); the volatiles were removed and the crude residue was purified by flash chromatography to give D as a white solid (1.6 g; 4.2 mmol). Yield 33%.

### Example VI

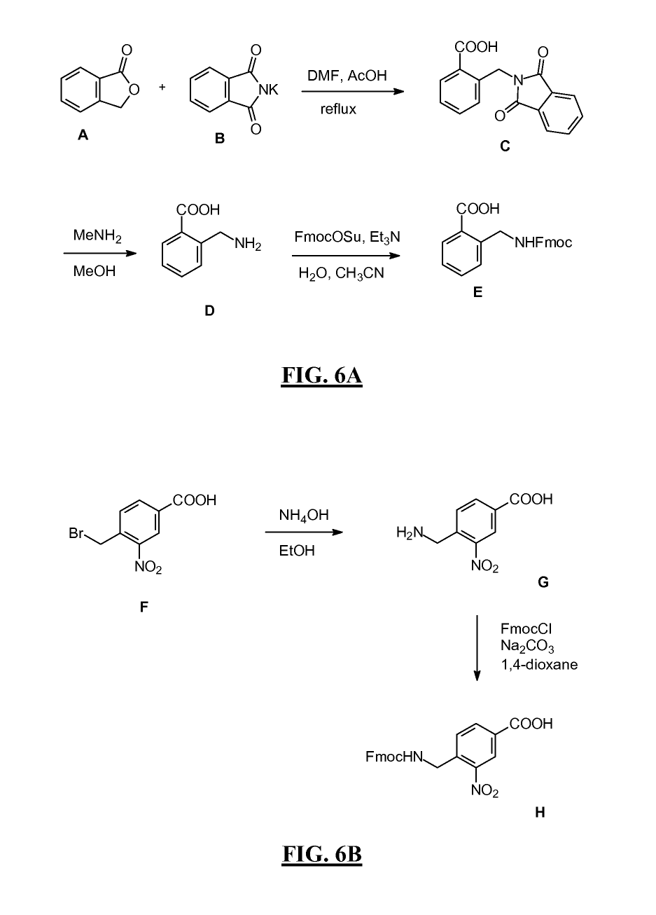

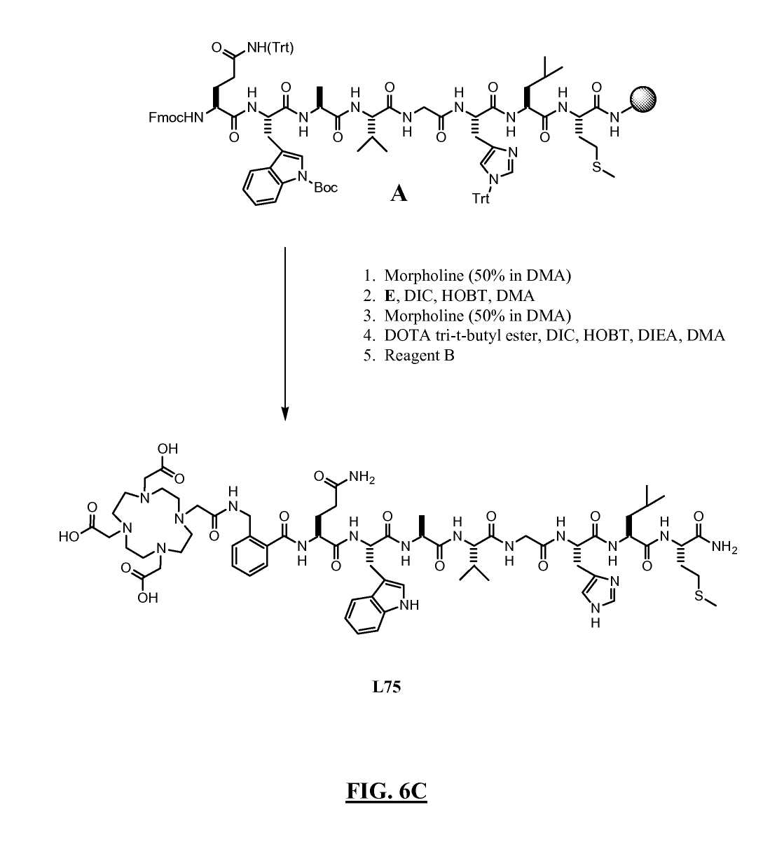

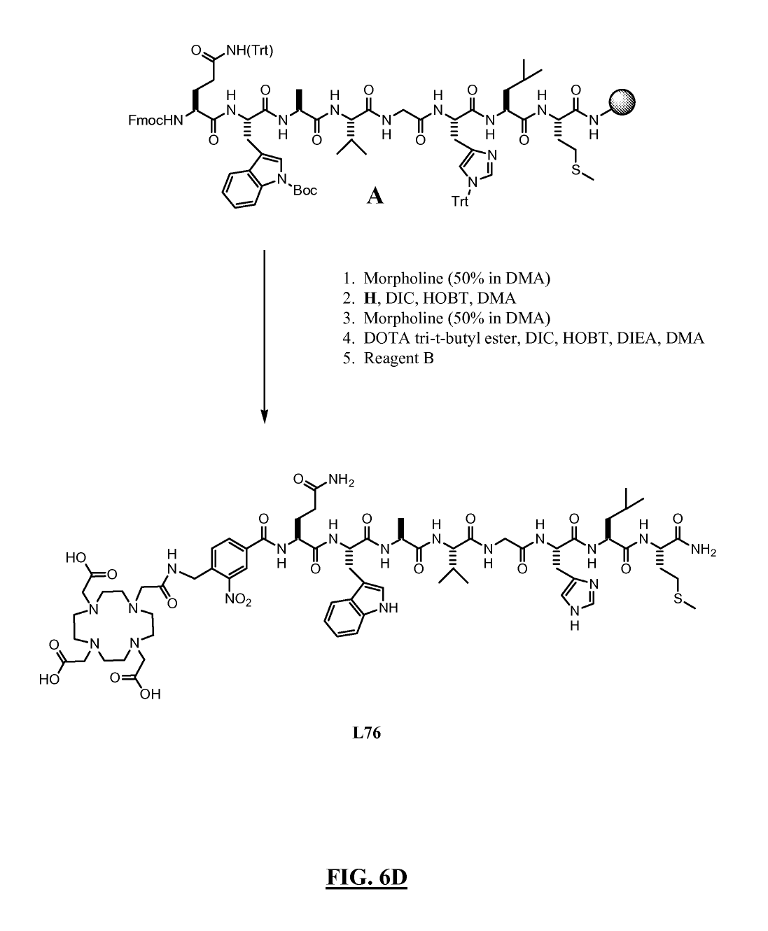

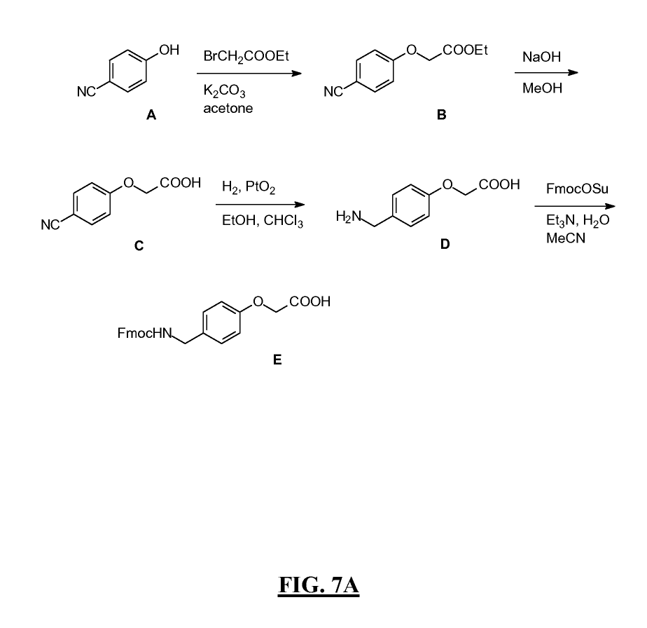

### FIGS. 6A-F

### Synthesis of L75 and L76

Summary: The two products were obtained by coupling of the octapeptide Gln-Trp-Ala-Val-Gly-His-Leu-Met-NH₂ (BBN[7-14], which is SEQ ID NO: 1) (A) on the Rink amide resin with the two linkers E and H, followed by functionalization with DOTA tri-t-butyl ester. After cleavage and deprotection with Reagent B the final products were purified by preparative HPLC. Overall yields: 8.5% (L75) and 5.6% (L76).

### A. 2-[(1,3-Dihydro-1,3-dioxo-2H-isoindol-2-yl)methyl]benzoic acid, (C) (FIG. 6A)

The product was synthesized following the procedure reported in the literature (Bornstein, J; Drummon, P. E.; Bedell, S. F. Org. Synth. Coll. Vol. IV 1963, 810-812).

### B. 2-(Aminomethyl)benzoic acid, (D) (FIG. 6A)

A 40% solution of methylamine (6.14 mL; 7.1 mmol) was added to 2-[(1,3-dihydro-1,3-dioxo-2H-isoindol-2-yl)methyl]benzoic acid C (2 g; 7.1 mmol) and then EtOH (30 mL) was added. After 5 minutes stirring at room temperature the reaction mixture was heated at 50° C. After 2.5 h, the mixture was cooled and the solvent was evaporated under reduced pressure. The crude product was suspended in 50 mL of absolute ethanol and the suspension was stirred at room temperature for 1 h. The solid was filtered and washed with EtOH to afford 2-(aminomethyl)benzoic acid D (0.87 g; 5.8 mmol). Yield 81%.

### C. 2-[[[9H-Fluoren-9-ylmethoxy)carbonyl]amino]methyl]benzoic acid, (E) (FIG. 6A)

The product was synthesized following the procedure reported in the literature (Sun, J-H.; Deneker, W. F. Synth. Commun. 1998, 28, 4525-4530).

### D. 4-(Aminomethyl)-3-nitrobenzoic acid, (G) (FIG. 6B)

4-(Bromomethyl)-3-nitrobenzoic acid (3.2 g; 12.3 mmol) was dissolved in 8% NH₃ in EtOH (300 mL) and the resulting solution was stirred at room temperature. After 22 h the solution was evaporated and the residue suspended in H₂O (70 mL). The suspension was stirred for 15 min and filtered. The collected solid was suspended in H₂O (40 mL) and dissolved by the addition of few drops of 25% aq. NH₄OH (pH 12), then the pH of the solution was adjusted to 6 by addition of 6 N HCl. The precipitated solid was filtered, and washed sequentially with MeOH (3×5 mL), and Et₂O (10 mL) and was vacuum dried (1.3 kPa; P₂O₅) to give 4-(aminomethyl)-3-nitrobenzoic acid as a pale brown solid (1.65 g; 8.4 mmol). Yield 68%.

### E. 4-[[[9H-Fluoren-9-ylmethoxy)carbonyl]amino]methyl]-3-nitrobenzoic acid, (H) (FIG. 6B)

4-(Aminomethyl)-3-nitrobenzoic acid G (0.8 g; 4 mmol) was dissolved in 10% aq. Na₂CO₃ (25 mL) and 1,4-dioxane (10 mL) and the solution was cooled to 0° C. A solution of 9-fluorenylmethyl chloroformate (Fmoc-C1) (1.06 g; 4 mmol) in 1,4-dioxane (10 mL) was added dropwise for 20 min. After 2 h at 0-5° C. and 1 h at 10° C. the reaction mixture was filtered and the solution was acidified to pH 5 by addition of 1 N HCl. The precipitate was filtered, washed with H₂O (2×2 mL) dried under vacuum (1.3 kPa; P₂O₅) to give 4-[[[9H-fluoren-9-ylmethoxy)carbonyl]amino]methyl]-3-nitrobenzoic acid as a white solid (1.6 g; 3.7 mmol). Yield 92%.

### F. L75 (N-[2-[[[[4,7,10-Tris(carboxymethyl)-1,4,7,10-tetraazacyclododec-1-yl]acetyl]amino]methyl]benzoyl]-L-glutaminyl-L-tryptophyl-L-alanyl-L-valyl-glycyl-L-histidyl-L-leucyl-L-methioninamide) (FIG. 6C)

Resin A (0.5 g; 0.3 mmol) was shaken in a solid phase peptide synthesis vessel with 50% morpholine in DMA (7 mL) for 10 min, the solution was emptied and fresh 50% morpholine in DMA (7 mL) was added. The suspension was stirred for 20 min then the solution was emptied and the resin washed with DMA (5×7 mL). 2-[[[9H-Fluoren-9-ylmethoxy)carbonyl]amino]methyl]benzoic acid, E (0.45 g; 1.2 mmol), N-hydroxybenzotriazole (HOBt) (0.18 g; 1.2 mmol), N,N′-diisopropylcarbodiimide (DIC) (0.19 mL; 1.2 mmol) and DMA (7 mL) were added to the resin, the mixture shaken for 24 h at room temperature, the solution was emptied and the resin was washed with DMA (5×7 mL). The resin was then shaken with 50% morpholine in DMA (7 mL) for 10 min, the solution was emptied, fresh 50% morpholine in DMA (7 mL) was added and the mixture shaken for min. The solution was emptied and the resin washed with DMA (5×7 mL). 1,4,7,10-Tetraazacyclododecane-1,4,7,10-tetraacetic acid tris(1,1-dimethylethyl) ester adduct with NaCl (DOTA tri-t-butyl ester) (0.79 g; 1.2 mmol), HOBt (0.18 g; 1.2 mmol), DIC (0.19 mL: 1.2 mmol), DIEA (0.40 mL; 2.4 mmol) and DMA (7 mL) were added to the resin. The mixture was shaken for 24 h at room temperature, the solution was emptied and the resin was washed with DMA (5×7 mL), CH₂Cl₂ (5×7 mL) and vacuum dried. The resin was shaken in a flask with Reagent B (25 mL) for 4.5 h. The resin was filtered and the filtrate was evaporated under reduced pressure to afford an oily crude that after treatment with Et₂O (20 mL) gave a precipitate. The resulting precipitate was collected by centrifugation and was washed with Et₂O (3×20 mL) to give L75 (190 mg; 0.13 mmol) as a white solid. Yield 44%.

### G. L76 (N-[4-[[[[4,7,10-Tris(carboxymethyl)-1,4,7,10-tetraazacyclododec-1-yl]acetyl]amino]methyl]-3-nitrobenzoyl]-L-glutaminyl-L-tryptophyl-L-alanyl-L-valyl-glycyl-L-histidyl-L-leucyl-L-methioninamide) (FIG. 6D)

Resin A (0.5 g; 0.3 mmol) was shaken in a solid phase peptide synthesis vessel with 50% morpholine in DMA (7 mL) for 10 min, the solution was emptied and fresh 50% morpholine in DMA (7 mL) was added. The suspension was stirred for 20 min then the solution was emptied and the resin was washed with DMA (5×7 mL). 4-[[[9H-Fluoren-9-ylmethoxy)carbonyl]amino]methyl]-3-nitrobenzoic acid, H (0.50 g; 1.2 mmol), HOBt (0.18 g; 1.2 mmol), DIC (0.19 mL; 1.2 mmol) and DMA (7 mL) were added to the resin, the mixture was shaken for 24 h at room temperature, the solution was emptied and the resin was washed with DMA (5×7 mL). The resin was then shaken with 50% morpholine in DMA (7 mL) for min, the solution was emptied, fresh 50% morpholine in DMA (7 mL) was added and the mixture was shaken for 20 min. The solution was emptied and the resin was washed with DMA (5×7 mL). DOTA tri-t-butyl ester (0.79 g; 1.2 mmol), HOBt (0.18 g; 1.2 mmol), DIC (0.19 mL: 1.2 mmol), DIEA (0.40 mL; 2.4 mmol) and DMA (7 mL) were added to the resin. The mixture was shaken for 24 h at room temperature, the solution was emptied and the resin was washed with DMA (5×7 mL), CH₂Cl₂ (5×7 mL) and vacuum dried. The resin was shaken in a flask with Reagent B (25 mL) for 4.5 h. The resin was filtered and the solution was evaporated under reduced pressure to afford an oily crude that was triturated with Et₂O (20 mL). The precipitate was collected by centrifugation and was washed with Et₂O (3×20 mL) to give a solid (141 mg) which was analysed by HPLC. A 37 mg portion of the crude was purified by preparative HPLC. The fractions containing the product were lyophilised to give L76 (10.8 mg; 7.2×10⁻³ mmol) as a white solid. Yield 9%.

### Example VII

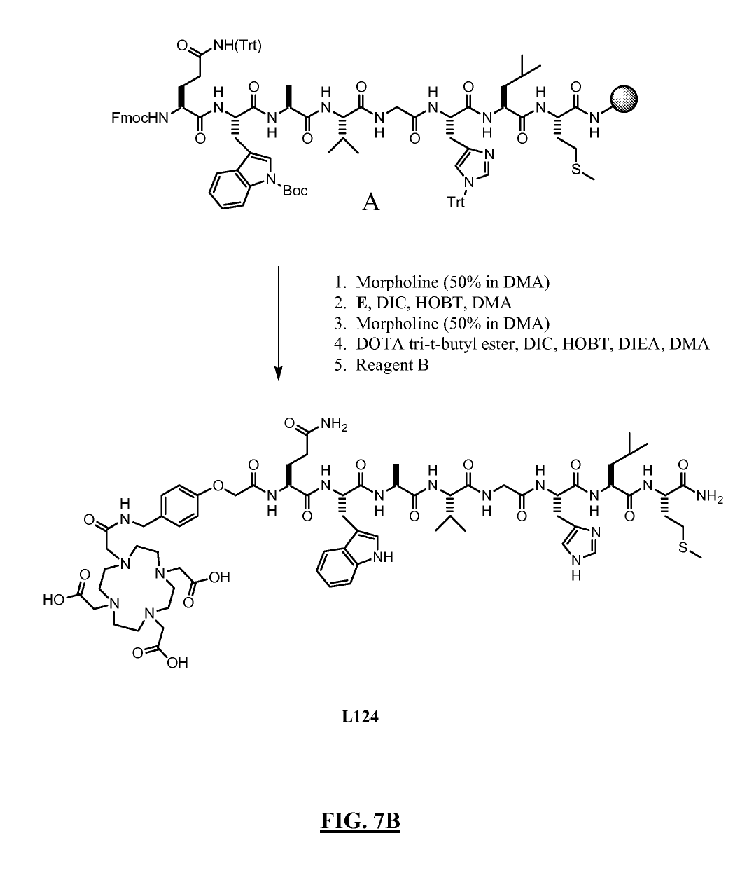

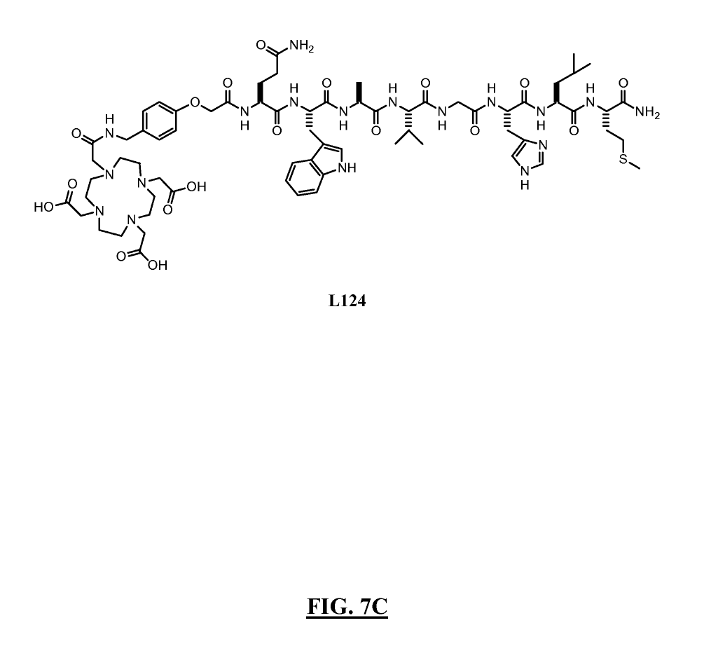

### FIGS. 7A-C

### Synthesis of L124

Summary: 4-Cyanophenol A was reacted with ethyl bromoacetate and K₂CO₃ in acetone to give the intermediate B, which was hydrolysed with NaOH to the corresponding acid C. Successive hydrogenation of C with H₂ and PtO₂ at 355 kPa in EtOH/CHCl₃ gave the corresponding aminoacid D, which was directly protected with FmocOSu to give E. Rink amide resin functionalised with the octapeptide Gln-Trp-Ala-Val-Gly-His-Leu-Met-NH₂ (BBN[7-14], which is SEQ ID NO:1) was reacted with E and then with DOTA tri-t-butyl ester. After cleavage and deprotection with Reagent B the crude was purified by preparative HPLC to give L124. Overall yield: 1.3%

### A. Synthesis of (4-Cyanophenoxy)acetic acid ethyl ester, (B) (FIG. 7A)

The product was synthesized following the procedure reported in the literature (Archimbault, P.; LeClerc, G.; Strosberg, A. D.; Pietri-Rouxel, F. PCT Int. Appl. WO 980005, 1998).

### B. Synthesis of (4-Cyanophenoxy)acetic acid, (C) (FIG. 7A)

A 1 N solution of NaOH (7.6 mL; 7.6 mmol) was added dropwise to a solution of (4-cyanophenoxy)acetic acid ethyl ester B (1.55 g; 7.6 mmol) in MeOH (15 mL). After 1 h the solution was acidified with 1 N HCl (7.6 mL; 7.6 mmol) and evaporated. The residue was taken up with water (20 mL) and extracted with CHCl₃ (2×30 mL). The organic phases were evaporated and the crude was purified by flash chromatography to give (4-cyanophenoxy)acetic acid C (0.97 g; 5.5 mmol) as a white solid. Yield 72%.

### C. Synthesis of [4-[[[9H-Fluoren-9-ylmethoxy)carbonyl]amino]methyl]phenoxy]acetic acid, (E) (FIG. 7A)

PtO₂ (150 mg) was added to a solution of (4-cyanophenoxy)acetic acid C (1.05 g; 5.9 mmol) in EtOH (147 mL) and CHCl₃ (3 mL). The suspension was stirred 30 h under a hydrogen atmosphere (355 kPa; 20° C.). The mixture was filtered through a Celite® pad and the solution evaporated under vacuum. The residue was purified by flash chromatography to give acid D (0.7 g) which was dissolved in H₂O (10 mL), MeCN (2 mL) and Et₃N (0.6 mL) at 0° C., then a solution of N-(9-fluorenylmethoxycarbonyloxy)succinimide (1.3 g; 3.9 mmol) in MeCN (22 mL) was added dropwise. After stirring 16 h at room temperature the reaction mixture was filtered and the volatiles were removed under vacuum. The residue was treated with 1 N HCl (10 mL) and the precipitated solid was filtered and purified by flash chromatography to give [4-[[[9H-fluoren-9-ylmethoxy)carbonyl]amino]methyl]phenoxy]acetic acid E (0.56 g; 1.4 mmol) as a white solid. Overall yield 24%.

### D. Synthesis of L124 (N-[[4-[[[[4,7,10-Tris(carboxymethyl)-1,4,7,10-tetraazacyclododec-1-yl]acetyl]amino]methyl]phenoxy]acetyl]-L-glutaminyl-L-tryptophyl-L-alanyl-L-valyl-glycyl-L-histidyl-L-leucyl-L-methioninamide)(FIG. 7B)

Resin A (480 mg; 0.29 mmol) was shaken in a solid phase peptide synthesis vessel with 50% morpholine in DMA (7 mL) for 10 min, the solution was emptied and fresh 50% morpholine in DMA (7 mL) was added. The suspension was stirred for 20 min, the solution was emptied and the resin was washed with DMA (5×7 mL). [4-[[[9H-Fluoren-9-ylmethoxy)carbonyl]amino]methyl]phenoxy]acetic acid E (480 mg; 1.19 mmol), N-hydroxybenzotriazole (HOBt) (182 mg; 1.19 mmol), N,N′-diisopropylcarbodiimide (DIC) (185 μL; 1.19 mmol) and DMA (7 mL) were added to the resin, the mixture was shaken for 24 h at room temperature, the solution was emptied and the resin was washed with DMA (5×7 mL). The resin was then shaken with 50% morpholine in DMA (6 mL) for 10 min, the solution was emptied, fresh 50% morpholine in DMA (6 mL) was added and the mixture was shaken for 20 min. The solution was emptied and the resin was washed with DMA (5×7 mL). 1,4,7,10-Tetraazacyclododecane-1,4,7,10-tetraacetic acid tris(1,1-dimethylethyl) ester adduct with NaCl (750 mg; 1.19 mmol), HOBt (182 mg; 1.19 mmol), DIEA (404 μL; 2.36 mmol), DIC (185 μL; 1.19 mmol) and DMA (6 mL) were added to the resin. The mixture was shaken for 24 h at room temperature, the solution was emptied, the resin was washed with DMA (2×7 mL), CH₂Cl₂ (5×7 mL) and vacuum dried. The resin was shaken in a flask with Reagent B (25 mL) for 4 h. The resin was filtered and the filtrate was evaporated under reduced pressure to afford an oily crude that was triturated with Et₂O (5 mL). The precipitate was collected by centrifugation and washed with Et₂O (5×5 mL) to give a solid (148 mg) which was analysed by HPLC. A 65 mg portion of the crude was purified by preparative HPLC. The fractions containing the product were lyophilised to give L124 (FIG. 7C) as a white solid (15 mg; 0.01 mmol). Yield 7.9%.

### Example VIII

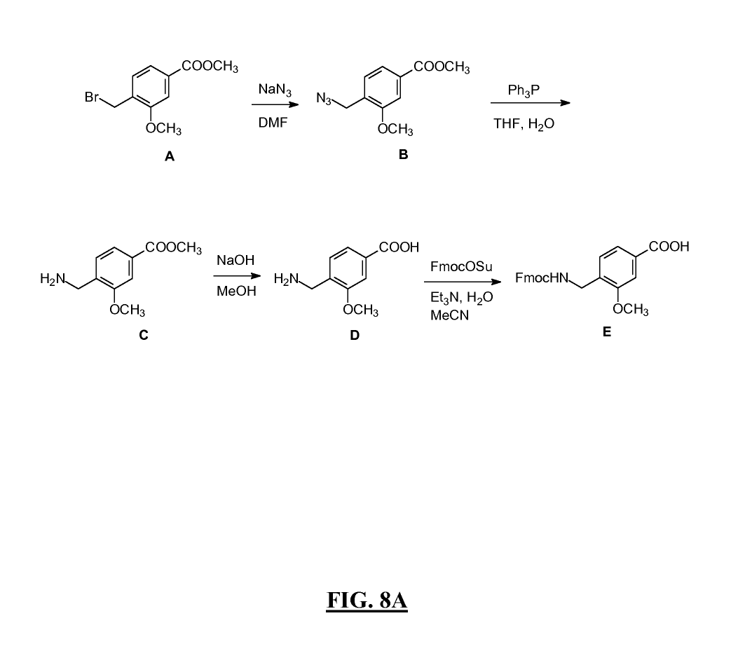

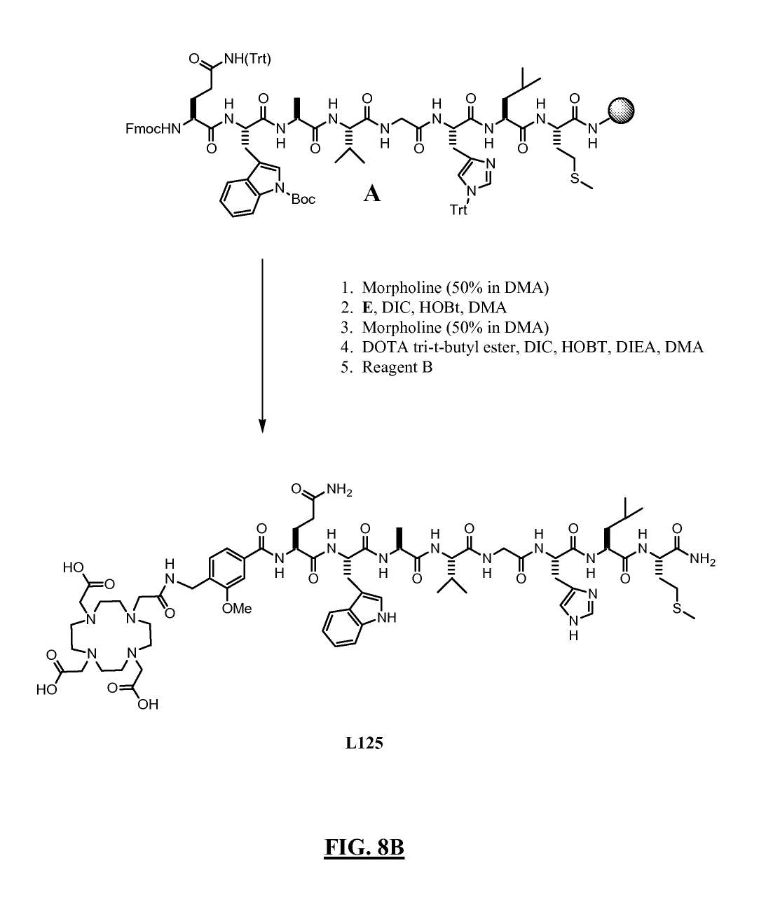

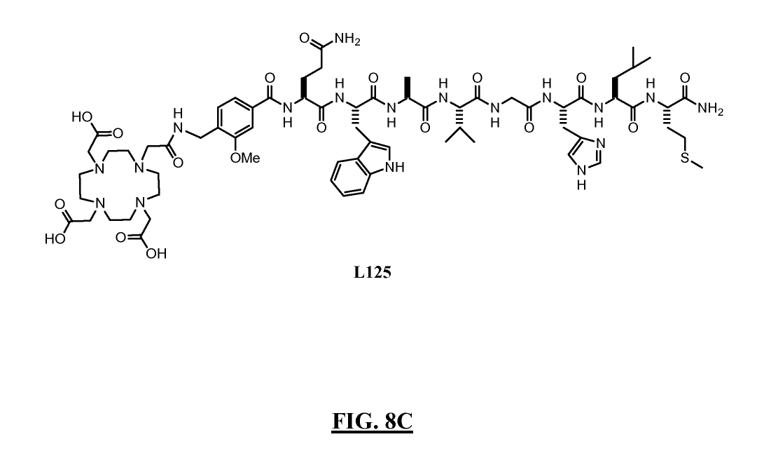

### FIGS. 8A-C

### Synthesis of L125

Summary: 4-(Bromomethyl)-3-methoxybenzoic acid methyl ester A was reacted with NaN₃ in DMF to give the intermediate azide B, which was then reduced with Ph₃P and H₂O to amine C. Hydrolysis of C with NaOH gave acid D, which was directly protected with FmocOSu to give E. Rink amide resin functionalised with the octapeptide Gln-Trp-Ala-Val-Gly-His-Leu-Met-NH₂ (BBN[7-14], which is SEQ ID NO:1) (A) was reacted with E and then with DOTA tri-t-butyl ester. After cleavage and deprotection with Reagent B the crude was purified by preparative HPLC to give L125. Overall yield: 0.2%.

### A. Synthesis of 4-(Azidomethyl)-3-methoxybenzoic acid methyl ester, (B) (FIG. 8A)

A solution of 4-(bromomethyl)-3-methoxybenzoic acid methyl ester (8 g; 31 mmol) and NaN₃ (2 g; 31 mmol) in DMF (90 mL) was stirred overnight at room temperature. The volatiles were removed under vacuum and the crude product was dissolved in EtOAc (50 mL). The solution was washed with water (2×50 mL) and dried. The volatiles were evaporated to provide 4-(azidomethyl)-3-methoxybenzoic acid methyl ester (6.68 g; 30 mmol). Yield 97%.

### B. 4-(Aminomethyl)-3-methoxybenzoic acid methyl ester, (C) (FIG. 8A)

Triphenylphosphine (6.06 g; 23 mmol) was added to a solution of (4-azidomethyl)-3-methoxybenzoic acid methyl ester B (5 g; 22 mmol) in THF (50 mL): hydrogen evolution and formation of a white solid was observed. The mixture was stirred under nitrogen at room temperature. After 24 h more triphenylphosphine (0.6 g; 2.3 mmol) was added. After 24 h the azide was consumed and H₂O (10 mL) was added. After 4 h the white solid disappeared. The mixture was heated at 45° C. for 3 h and was stirred overnight at room temperature. The solution was evaporated to dryness and the crude was purified by flash chromatography to give 4-(aminomethyl)-3-methoxybenzoic acid methyl ester C (1.2 g; 6.1 mmol). Yield 28%.

### C. 4-[[[9H-Fluoren-9-ylmethoxy)carbonyl]amino]methyl]-3-methoxybenzoic acid, (E) (FIG. 8A)

A 1 N solution of NaOH (6.15 mL; 6.14 mmol) was added dropwise to a solution of 4-(aminomethyl)-3-methoxybenzoic acid methyl ester C (1.2 g; 6.14 mmol) in MeOH (25 mL) heated at 40° C. After stirring 8 h at 45° C. the solution was stirred over night at room temperature. A 1 N solution of NaOH (0.6 mL; 0.6 mmol) was added and the mixture heated at 40° C. for 4 h. The solution was concentrated, acidified with 1 N HCl (8 mL; 8 mmol), extracted with EtOAc (2×10 mL) then the aqueous layer was concentrated to 15 mL. This solution (pH 4.5) was cooled at 0° C. and Et₃N (936 μL; 6.75 mmol) was added (pH 11). A solution of N-(9-fluorenylmethoxycarbonyloxy)succinimide (3.04 g; 9 mmol) in MeCN (30 mL) was added dropwise (final pH 9) and a white solid precipitated. After stirring 1 h at room temperature the solid was filtered, suspended in 1N HCl (15 mL) and the suspension was stirred for 30 min. The solid was filtered to provide 4-[[[9H-fluoren-9-ylmethoxy)carbonyl]amino]methyl]-3-methoxybenzoic acid E as a white solid (275 mg; 0.7 mmol).

The filtrate was evaporated under vacuum and the resulting white residue was suspended in 1N HCl (20 mL) and stirred for 30 minutes. The solid was filtered and purified by flash chromatography to give more acid E (198 mg; 0.5 mmol). Overall yield 20%.

### D. L125 (N-[4-[[[[4,7,10-Tris(carboxymethyl)-1,4,7,10-tetraazacyclododec-1-yl]acetyl]amino]methyl]-3-methoxybenzoyl]-L-glutaminyl-L-tryptophyl-L-alanyl-L-valyl-glycyl-L-histidyl-1L-methioninamide) (FIG. 8B)

Resin A (410 mg; 0.24 mmol) was shaken in a solid phase peptide synthesis vessel with 50% morpholine in DMA (7 mL) for 10 min, the solution was emptied and fresh 50% morpholine in DMA (7 mL) was added. The suspension was stirred for 20 min then the solution was emptied and the resin was washed with DMA (5×7 mL). 4-[[[9H-Fluoren-9-ylmethoxy)carbonyl]amino]methyl]-3-methoxybenzoic acid E (398 mg; 0.98 mmol), HOBt (151 mg; 0.98 mmol), DIC (154 μL; 0.98 mmol) and DMA (6 mL) were added to the resin; the mixture was shaken for 24 h at room temperature, the solution was emptied and the resin was washed with DMA (5×7 mL). The resin was then shaken with 50% morpholine in DMA (6 mL) for 10 min, the solution was emptied, fresh 50% morpholine in DMA (6 mL) was added and the mixture was shaken for 20 min. The solution was emptied and the resin washed with DMA (5×7 mL). 1,4,7,10-Tetraazacyclododecane-1,4,7,10-tetraacetic acid tris(1,1-dimethylethyl) ester adduct with NaCl (618 mg; 0.98 mmol), HOBt (151 mg; 0.98 mmol), DIC (154 μL; 0.98 mmol), DIEA (333 μL; 1.96 mmol) and DMA (6 mL) were added to the resin. The mixture was shaken for 24 h at room temperature, the solution was emptied and the resin was washed with DMA (5×7 mL), CH₂Cl₂ (5×7 mL) and vacuum dried. The resin was shaken in a flask with Reagent B (25 mL) for 4 h. The resin was filtered and the solution was evaporated under reduced pressure to afford an oily crude that was triturated with Et₂O (5 mL). The resulting precipitate was collected by centrifugation, was washed with Et₂O (5×5 mL), was analysed by HPLC and purified by preparative HPLC. The fractions containing the product were lyophilised to give L125 (FIG. 8C) as a white solid (15.8 mg; 0.011 mmol). Yield 4.4%.

### Example IX

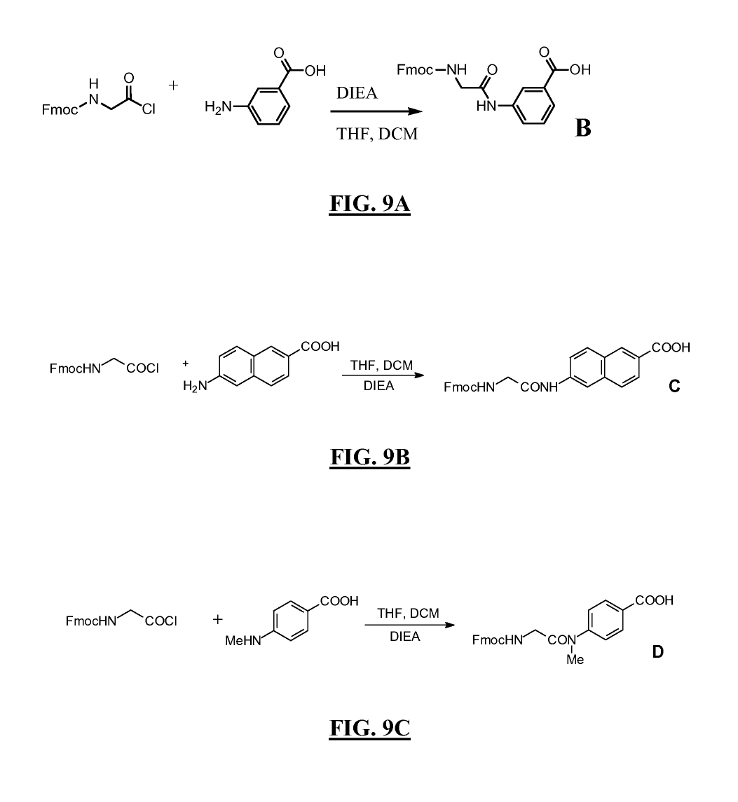

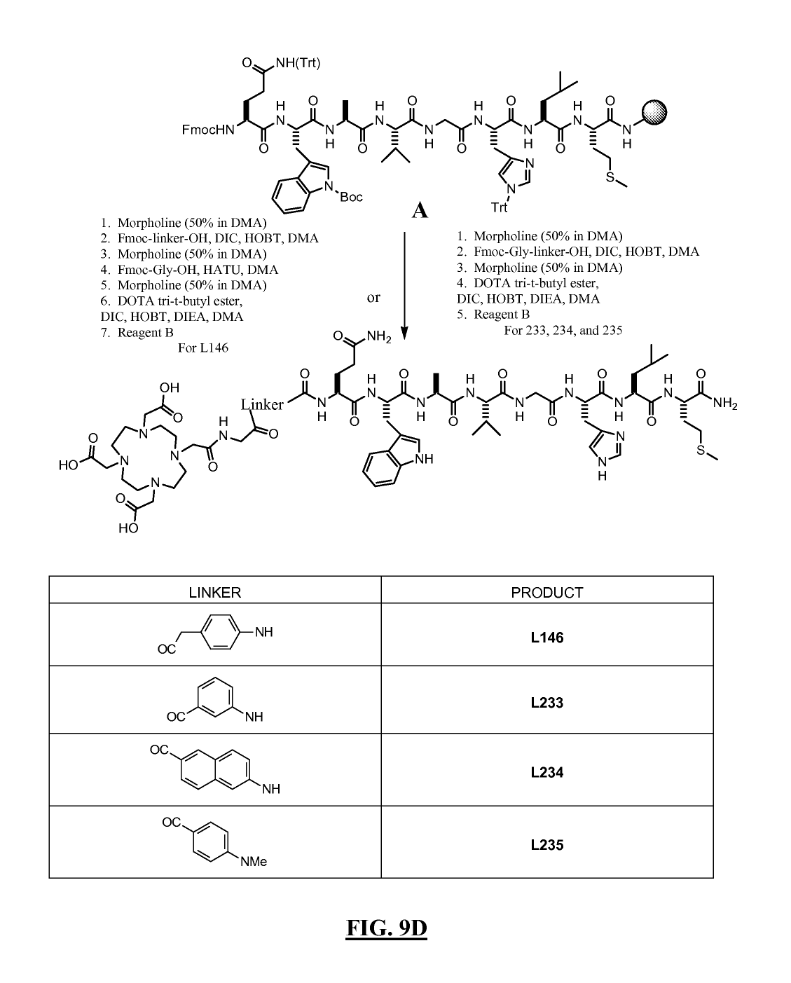

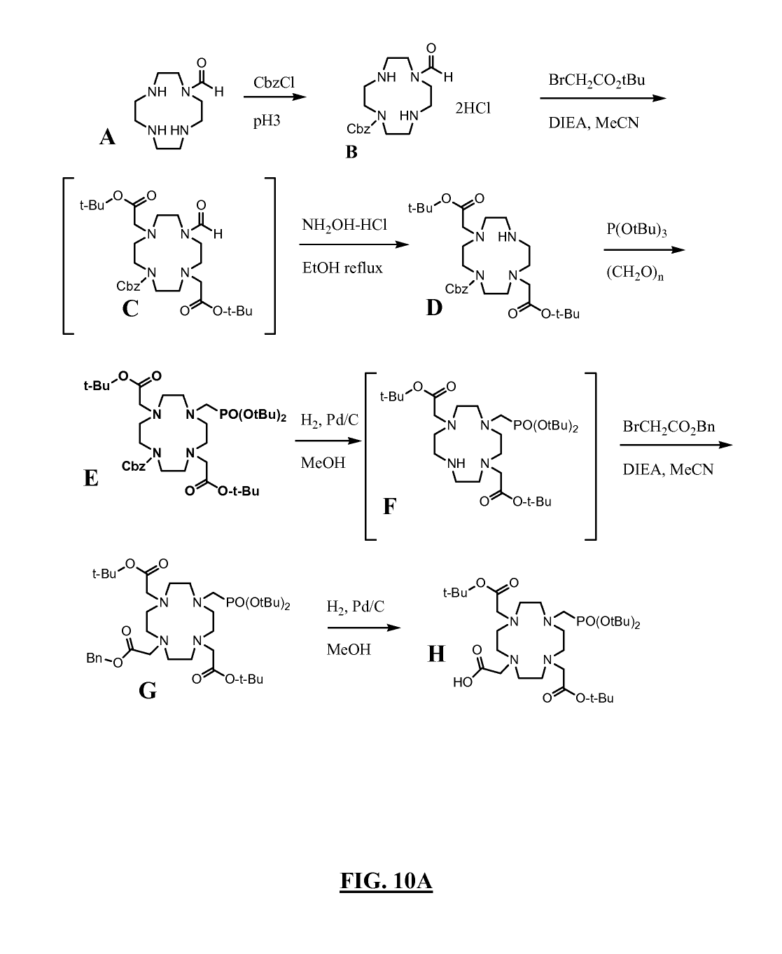

### FIGS. 9A-9D

### Synthesis of L146, L233, L234, and L235

Summary: The products were obtained in several steps starting from the octapeptide Gln-Trp-Ala-Val-Gly-His-Leu-Met-NH₂(BBN[7-14]) (A) on the Rink amide resin. After final cleavage and deprotection with Reagent B the crudes were purified by preparative HPLC to give L146, L233, L234 and L235. Overall yields: 10%, 11%, 4.5%, 5.7% respectively.

### A. 3-[[[(9H-Fluoren-9-ylmethoxy)carbonyl]amino]acetyl]aminobenzoic acid, B (FIG. 9A)

A solution of 3-aminobenzoic acid (0.5 g; 3.8 mmol) and N-ethyldiisopropylamine (DIEA) (0.64 mL; 3.8 mmol) in THF (20 mL) was added dropwise to a solution of Fmoc-glycine chloride (1.2 g; 4.0 mmol) (3) in THF (10 mL) and CH₂Cl₂ (10 mL). After 24 h stirring at room temperature 1 M HCl (50 mL) was added (final pH: 1.5). The precipitate was filtered, washed with H₂O (2×100 mL), vacuum dried and crystallised from CHCl₃/CH₃OH (1:1) to give B as a white solid (0.7 g; 1.6 mmol). Yield 43%.

### B. N-[3-[[[[[4,7,10-Tris(carboxymethyl)-1,4,7,10-tetraazacyclododec-1-yl]acetyl]amino]acetyl]amino]benzoyl]-L-glutaminyl-L-tryptophyl-L-alanyl-L-valyl-glycyl-L-histidvl-L-leucyl-L-methioninamide, L233 (FIG. 9D)

Resin A (0.5 g; 0.3 mmol) was shaken in a solid phase peptide synthesis vessel with 50% morpholine in DMA (7 mL) for 10 min, the solution emptied and fresh 50% morpholine in DMA (7 mL) was added.

The suspension was stirred for another 20 min then the solution was emptied and the resin washed with DMA (5×7 mL). 3-[[[(9H-Fluoren-9-ylmethoxy)carbonyl]amino]acetyl]aminobenzoic acid, B (0.50 g; 1.2 mmol), HOBt (0.18 g; 1.2 mmol), DIC (0.19 mL; 1.2 mmol) and DMA (7 mL) were added to the resin, the mixture shaken for 6 h at room temperature, emptied and the resin washed with DMA (5×7 mL). The resin was then shaken with 50% morpholine in DMA (7 mL) for 10 min, the solution emptied, fresh 50% morpholine in DMA (7 mL) was added and the mixture shaken for another 20 min. The solution was emptied and the resin washed with DMA (5×7 mL). DOTA tri-t-butyl ester adduct with NaCl² (0.79 g; 1.2 mmol) (5), HOBt (0.18 g; 1.2 mmol), DIC (0.19 mL: 1.2 mmol), DIEA (0.40 mL; 2.4 mmol) and DMA (7 mL) were added to the resin. The mixture was shaken for 24 h at room temperature, emptied and the resin washed with DMA (5×7 mL), CH₂Cl₂ (5×7 mL) and vacuum dried. The resin was shaken in a flask with Reagent B (25 mL) for 4.5 h. The resin was filtered and the solution was evaporated under reduced pressure to afford an oily crude that after treatment with Et₂O (20 mL) gave a precipitate. The precipitate was collected by centrifugation and washed with Et₂O (3×20 mL) to give a solid (152 mg) which was analysed by HPLC. An amount of crude (50 mg) was purified by preparative HPLC. The fractions containing the product were lyophilised to give L233 (17.0 mg; 11.3×10⁻³ mmol) as a white solid. Yield 11%.

### C. N-[4-[[[[[4,7,10-Tris(carboxymethyl)-1,4,7,10-tetraazacyclododec-1-yl]acetyl]amino]acetyl]amino]phenylacetyl]-L-glutaminyl-L-tryptophyl-L-alanyl-L-valyl-glycyl-L-histidyl-L-leucyl-L-methioninamide, L146 (FIG. 9D)

Resin A (0.5 g; 0.3 mmol) was shaken in a solid phase peptide synthesis vessel with 50% morpholine in DMA (7 mL) for 10 min, the solution filtered and fresh 50% morpholine in DMA (7 mL) was added. The suspension was stirred for another 20 min then the solution was filtered and the resin washed with DMA (5×7 mL). Fmoc-4-aminophenylacetic acid (0.45 g; 1.2 mmol), HOBt (0.18 g; 1.2 mmol), DIC (0.19 mL; 1.2 mmol) and DMA (7 mL) were added to the resin, the mixture shaken for 6 h at room temperature, filtered and the resin washed with DMA (5×7 mL). The resin was then shaken with 50% morpholine in DMA (7 mL) for 10 min, the solution filtered, fresh 50% morpholine in DMA (7 mL) was added and the mixture shaken for another 20 min. The solution was filtered and the resin washed with DMA (5×7 mL). Fmoc-glycine (0.36 g; 1.2 mmol), HATU (0.46 g; 1.2 mmol) and DIEA (0.40 mL; 2.4 mmol) were stirred for 15 min in DMA (7 mL) then the solution was added to the resin, the mixture shaken for 2 h at room temperature, filtered and the resin washed with DMA (5×7 mL). The resin was then shaken with 50% morpholine in DMA (7 mL) for 10 min, the solution filtered, fresh 50% morpholine in DMA (7 mL) was added and the mixture shaken for another 20 min. The solution was filtered and the resin washed with DMA (5×7 mL). DOTA tri-t-butyl ester adduct with NaCl (0.79 g; 1.2 mmol), HOBt (0.18 g; 1.2 mmol), DIC (0.19 mL; 1.2 mmol), DIEA (0.40 mL; 2.4 mmol) and DMA (7 mL) were added to the resin. The mixture was shaken for 24 h at room temperature, filtered and the resin washed with DMA (5×7 mL), CH₂Cl₂ (5×7 mL) and vacuum dried. The resin was shaken in a flask with Reagent B (25 mL) for 4.5 h. The resin was filtered and the solution was evaporated under reduced pressure to afford an oily crude that after treatment with Et₂O (20 mL) gave a precipitate. The precipitate was collected by centrifugation and washed with Et₂O (3×20 mL) to give a solid (203 mg) which was analysed by HPLC. An amount of crude (50 mg) was purified by preparative HPLC. The fractions containing the product were lyophilised to give L146 (11.2 mg; 7.4×10⁻³ mmol) as a white solid. Yield 10%.

### D. 6-[[[(9H-Fluoren-9-ylmethoxy)carbonyl]amino]acetyl]aminonaphthoic acid, C (FIG. 9B)

A solution of 6-aminonaphthoic acid (500 mg; 2.41 mmol); and DIEA (410 μL 2.41 mmol) in THF (20 mL) was added dropwise to a solution of Fmoc-glycine chloride (760 mg; 2.41 mmol) in CH₂Cl₂/THF 1:1 (10 mL) and stirred at room temperature. After 24 h the solvent was evaporated under vacuum. The residue was taken up with 0.5 N HCl (50 mL) and stirred for 1 h. The white solid precipitated was filtered and dried. The white solid was suspended in methanol (30 mL) and boiled for 5 min, then was filtered to give product C (690 mg; 1.48 mmol). Yield 62%.

### E. N-[6-[[[[[4,7,10-Tris(carboxymethyl)-1,4,7,10-tetraazacyclododec-1-yl]acetyl]amino]acetyl]amino]naphthoyl]-L-glutaminyl-L-tryptophyl-L-alanyl-L-valyl-glycyl-L-histidyl-L-leucyl-L-methioninamide, L234

Resin A (500 mg; 0.3 mmol) was shaken in a solid phase peptide synthesis vessel with 50% morpholine in DMA (7 mL) for 10 min, the solution emptied and fresh 50% morpholine in DMA (7 mL) was added. The suspension was stirred for another 20 min then the solution was emptied and the resin washed with DMA (5×7 mL). 6-[[[(9H-Fluoren-9-ylmethoxy)carbonyl]amino]acetyl]aminonaphthoic acid C (560 mg; 1.2 mmol), HOBt (184 mg; 1.2 mmol), DIC (187 μL; 1.2 mmol) and DMA (7 mL) were added to the resin, the mixture shaken for 6 h at room temperature, emptied and the resin washed with DMA (5×7 mL). The resin was then shaken with 50% morpholine in DMA (6 L) for 10 min, the solution emptied, fresh 50% morpholine in DMA (7 mL) was added and the mixture shaken for another 20 min. The solution was emptied and the resin washed with DMA (5×7 mL). DOTA tri-t-butyl ester adduct with NaCl (757 mg; 1.2 mmol), HOBt (184 mg; 1.2 mmol), DIC (187 μL; 1.2 mmol), and DIEA (537 μL; 2.4 mmol) and DMA (7 mL) were added to the resin. The mixture was shaken in a flask, emptied and the resin washed with DMA (2×7 mL), CH₂Cl₂ (5×7 mL) and vacuum dried. The resin was shaken in a flask with Reagent B (25 mL) for 4.5 h. The resin was filtrated and the solution was evaporated under reduced pressure to afford an oil crude that after treatment with Et₂O (20 mL) gave a precipitate. The precipitate was collected by centrifugation and washed with Et₂O (3×20 mL) to give a solid (144 mg) which was analysed by HPLC. An amount of crude (54 mg) was purified by preparative HPLC. The fractions containing the product were lyophilised to give L234 (8 mg; 5.1×10⁻³ mmol) as a white solid. Yield 4.5%.

### F. 4-[[[[(9H-Fluoren-9-ylmethoxy)carbonyl]amino]acetyl]methylamino]benzoic acid, D (FIG. 9C)

A solution of 4-N-methylaminonaphthoic acid (500 mg; 3.3 mmol) and DIEA (562 μL 3.3 mmol) in THF (20 mL) was added to a solution of Fmoc-glycine chloride (1.04 g; 3.3 mmol) in CH₂Cl₂/THF 1:1 (10 mL) and stirred at room temperature. After 24 h the solvent was evaporated under vacuum. The residue was taken up with 0.5 N HCl (30 mL) and was stirred for 3 h at 0° C. The white solid precipitated was filtered and dried. The crude was purified by flash chromatography to give Compound D (350 mg; 0.81 mmol). Yield 25%.

### G. N-[4-[[[[[4,7,10-Tris(carboxymethyl)-1,4,7,10-tetraazacyclododec-1-yl]acetyl]amino]acetyl]methylamino]benzoyl]-L-glutaminyl-L-tryptophyl-L-alanyl-L-valyl-glycyl-L-histidyl-L-leucyl-L-methioninamide, L235 (FIG. 9D)

Resin A (500 mg; 0.3 mmol) was shaken in a solid phase peptide synthesis vessel with 50% morpholine in DMA (7 mL) for 10 min, the solution emptied and fresh 50% morpholine in DMA (7 mL) was added. The suspension was stirred for another 20 min then the solution was emptied and the resin washed with DMA (5×7 mL). 4-[[[[9H-Fluoren-9-ylmethoxy)carbonyl]amino]acetyl]-N-methyl]amino-benzoic acid D (510 mg; 1.2 mmol), HOBt (184 mg; 1.2 mmol), DIC (187 μL; 1.2 mmol) and DMA (7 mL) were added to the resin, the mixture shaken for 6 h at room temperature, emptied and the resin washed with DMA (5×7 mL). The resin was then shaken with 50% morpholine in DMA (7 mL) for 10 min, the solution emptied, fresh 50% morpholine in DMA (7 mL) was added and the mixture shaken for another 20 min. The solution was emptied and the resin washed with DMA (5×7 mL). DOTA tri-t-butyl ester adduct with NaCl (757 mg; 1.2 mmol), HOBt (184 mg; 1.2 mmol), DIC (187 μL; 1.2 mmol), and DIEA (537 μL; 2.4 mmol) and DMA (7 mL) were added to the resin. The mixture was shaken in a flask, emptied and the resin washed with DMA (2×7 mL), CH₂Cl₂ (5×7 mL) and vacuum dried. The resin was shaken in a flask with Reagent B (25 mL) for 4.5 h. The resin was filtrated and the solution was evaporated under reduced pressure to afford an oil crude that after treatment with Et₂O (20 mL) gave a precipitate.

The precipitate was collected by centrifugation and washed with Et₂O (3×20 mL) to give a solid (126 mg) which was analysed by HPLC. An amount of crude (53 mg) was purified by preparative HPLC. The fractions containing the product were lyophilised to give L235 (11 mg; 7.2×10⁻³ mmol) as a white solid. Yield 5.7%.

### Example X

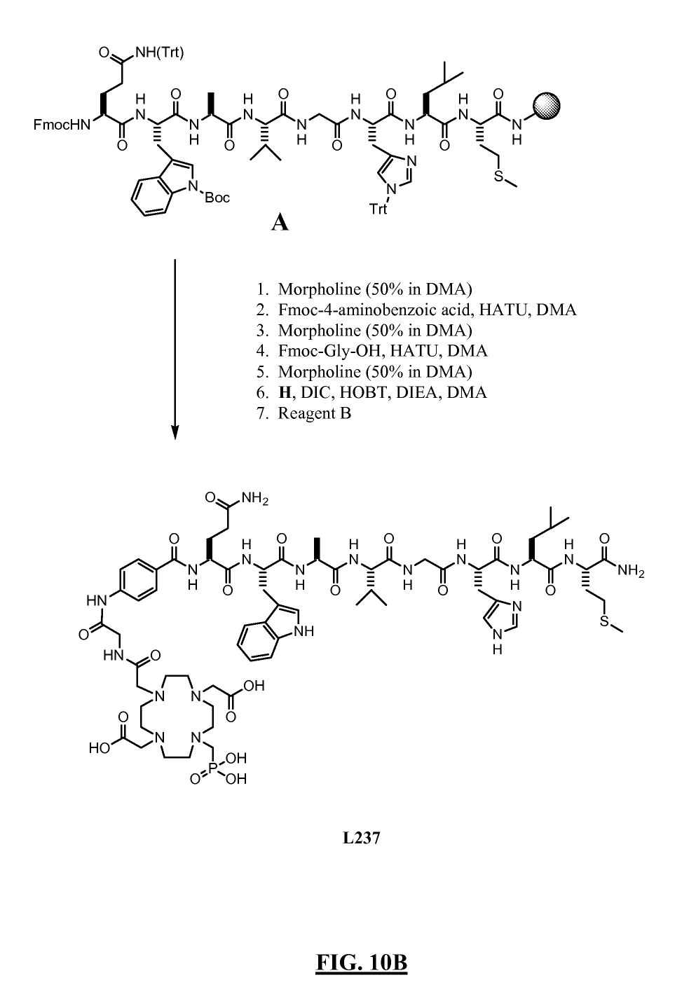

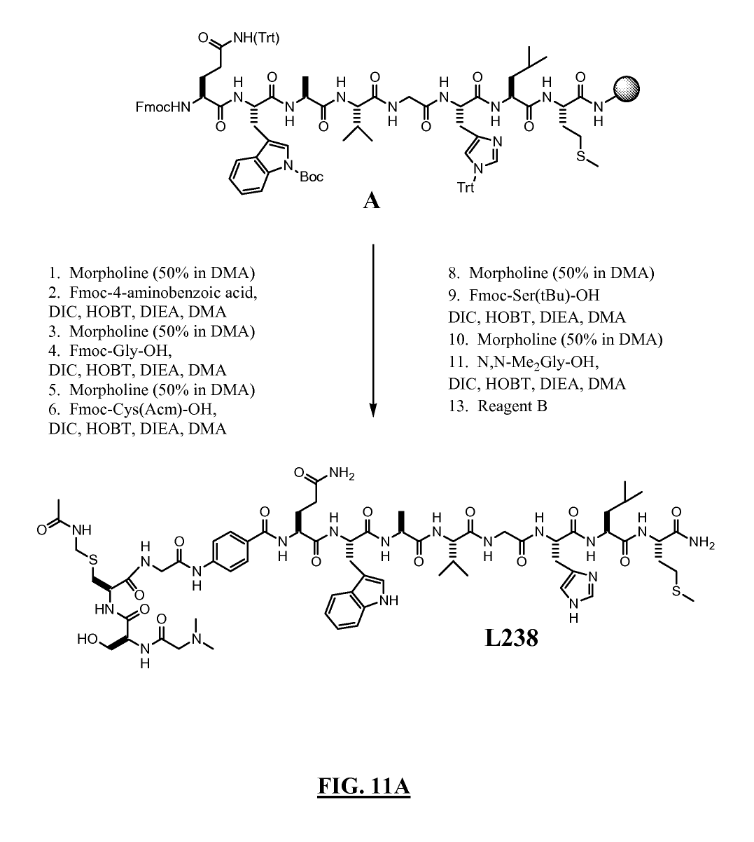

### FIGS. 10A-B

### Synthesis of L237

Summary: 1-Formyl-1,4,7,10-tetraazacyclododecane (A) was selectively protected with benzyl chloroformate at pH 3 to give B, which was alkylated with t-butyl bromoacetate and deformylated with hydroxylamine hydrochloride to give D. Reaction with P(OtBu)₃ and paraformaldehyde gave E, which was deprotected by hydrogenation and alkylated with benzyl bromoacetate to give G, which was finally hydrogenated to H. Rink amide resin functionalized with the octapeptide Gln-Trp-Ala-Val-Gly-His-Leu-Met-NH₂ (BBN[7-14]) (A) was sequentially reacted with Fmoc-4-aminobenzoic acid, Fmoc-glycine and H. After cleavage and deprotection with Reagent B the crude was purified by preparative HPLC to give L237. Overall yield 0.21%.

### A. 7-Formyl-1,4,7,10-tetraazacyclododecane-1-carboxylic acid phenylmethyl ester dihydrochloride, B (FIG. 10A)

1-Formyl-1,4,7,10-tetraazacyclododecane A (14 g; 69.9 mmol) was dissolved in H₂O (100 mL) and 12 N HCl (11 mL) was added until pH 3 then 1,4-dioxane (220 mL) was added. A solution of benzyl chloroformate (13.8 g; 77 mmol) in 1,4-dioxane (15 mL) was slowly added dropwise in 3.5 h, constantly maintaining the reaction mixture at pH 3 by continuous addition of 2 N NaOH (68.4 mL) with a pHstat apparatus. At the end of the addition the reaction was stirred for 1 h then washed with n-hexane (4×100 mL) and ⁱPr₂O (4×100 mL). The aqueous phase was brought to pH 13 by addition of 10 N NaOH (6.1 mL) and extracted with CHCl₃ (4×100 mL). The organic phase was washed with brine (100 mL), dried (Na₂SO₄), filtered and evaporated. The oily residue was dissolved in acetone (200 mL) and 6 N HCl (26 mL) was added. The solid precipitated was filtered, washed with acetone (2×50 mL) and dried under vacuum to give compound B (23.6 g; 58 mmol) as a white crystalline solid. Yield 83%.

### B. 4-(Phenylmethoxy)carbonyl-1,4,7,10-tetraazacyclododecane-1,7-diacetic acid bis(1,1-dimethylethyl) ester, D (FIG. 10A)

A solution of B (14.4 g; 35.3 mmol) in H₂O (450 mL) and 1 N NaOH (74 mL; 74 mmol) was stirred for 20 min then extracted with CHCl₃ (4×200 mL). The organic layer was evaporated to obtain an oily residue (12.3 g) which was dissolved in CH₃CN (180 mL) and N-ethyldiisopropylamine (DIEA) (15 mL; 88.25 mmol). A solution of t-butyl bromoacetate (16.8 g; 86.1 mmol) in CH₃CN (15 mL) was added dropwise to the previous solution in 2.5 h. After 20 h at room temperature the solvent was evaporated and the oily residue was dissolved in CHCl₃ (150 mL) and washed with H₂O (5×100 mL). The organic layer was dried (Na₂SO₄), filtered and evaporated to dryness to give C as a yellow oil. Crude C (22 g) was dissolved in EtOH (250 mL), NH₂OH.HCl (2.93 g; 42.2 mmol) was added and the solution heated to reflux. After 48 h the solvent was evaporated and the residue dissolved in CH₂Cl₂ (250 mL), washed with H₂O (3×250 mL) then with brine (3×250 mL). The organic layer was dried (Na₂SO₄), filtered and evaporated. The oily residue (18.85 g) was purified by flash chromatography. The fractions containing the product were collected and evaporated to obtain a glassy white solid (17.62 g) which was dissolved in H₂O (600 mL) and 1 N NaOH (90 mL; 90 mmol) and extracted with CHCl₃ (3×250 ml). The organic layer was dried (Na₂SO₄) and evaporated to dryness to give D (16.6 g; 31 mmol) as an oil. Yield 88%.

### C. 4-(Phenylmethoxy)carbonyl-10-[[bis(1,1-dimethylethoxy)phosphinyl]methyl]-1,4,7,10-tetraazacyclododecane-1,7-diacetic acid bis(1,1-dimethylethyl) ester, E (FIG. 10A)

A mixture of Compound D (13.87 g; 26 mmol), P(OtBu)₃ (7.6 g; 28.6 mmol) (10) and paraformaldeyde (0.9 g; 30 mmol) was heated at 60° C. After 16 h more P(OtBu)₃ (1 g; 3.76 mmol) and paraformaldeyde (0.1 g; 3.33 mmol) were added. The reaction was heated at 60° C. for another 20 h then at 80° C. for 8 h under vacuum to eliminate the volatile impurities. The crude was purified by flash chromatography to give E (9.33 g; 8 mmol) as an oil. Yield 31%.

### D. 7-[[Bis(1,1-dimethylethoxy)phosphinyl]methyl]-1,4,7,10-tetraazacyclododecane-1,4,10-triacetic acid 1-phenylmethyl 4,10-bis(1,1-dimethylethyl) ester, G (FIG. 10A)

To the solution of E (6.5 g; 5.53 mmol) in CH₃OH (160 mL) 5% Pd/C (1 g; 0.52 mmol) was added and the mixture was stirred under hydrogen atmosphere at room temperature. After 4 h (consumed H₂ 165 mL; 6.7 mmol) the mixture was filtered through a Millipore® filter (FT 0.45 μm) and the solution evaporated under reduced pressure. The crude (5.9 g) was purified by flash chromatography to give F (4.2 g) as an oil. Benzyl bromoacetate (1.9 g; 8.3 mmol) dissolved in CH₃CN (8 mL) was added dropwise in 1 h to a solution of F (4.2 g) in CH₃CN (40 mL) and DIEA (1.5 mL; 8.72 mmol). After 36 h at room temperature the solvent was evaporated and the residue (5.76 g) dissolved in CHCl₃ (100 mL), washed with H₂O (2×100 mL) then with brine (2×70 mL). The organic layer was dried (Na₂SO₄), filtered and evaporated. The crude (5.5 g) was purified twice by flash chromatography, the fractions were collected and evaporated to dryness to afford G (1.12 g; 1.48 mmol) as an oil. Yield 27%.

### E. 7-[[Bis(1,1-dimethylethoxy)phosphinyl]methyl]-1,4,7,10-tetraazacyclododecane-1,4,10-triacetic acid 4,10-bis(1,1-dimethylethyl) ester, H (FIG. 10A)

5% Pd/C (0.2 g; 0.087 mmol) was added to a solution of G (1.12 g; 1.48 mmol) in CH₃OH (27 mL) and the mixture was stirred under hydrogen atmosphere at room temperature. After 2 h (consumed H₂ 35 mL; 1.43 mmol) the mixture was filtered through a Millipore® filter (FT 0.45 μm) and the solution evaporated to dryness to give H (0.94 g; 1.41 mmol) as a pale yellow oil. Yield 97%.

### F. N-[4-[[[[[4,10-Bis(carboxymethyl)-7-(dihydroxyphosphinyl)methyl-1,4,7,10-tetraazacyclododec-1-yl]acetyl]amino]acetyl]amino]benzoyl]-L-glutaminyl-L-tryptophyl-L-alanyl-L-valyl-glycyl-L-histidyl-L-leucil-L-methioninamide, L237 (FIG. 10B)

Resin A (330 mg; 0.20 mmol) (17) was shaken in a solid phase peptide synthesis vessel with 50% morpholine in DMA (5 mL) for 10 min, the solution emptied and fresh 50% morpholine in DMA (5 mL) was added. The suspension was stirred for another min then the solution was emptied and the resin washed with DMA (5×5 mL). Fmoc-4-aminobenzoic acid (290 mg; 0.80 mmol), HOBt (120 mg; 0.80 mmol), DIC (130 μL; 0.80 mmol) and DMA (5 mL) were added to the resin, the mixture shaken for 3 h at room temperature, emptied and the resin washed with DMA (5×5 mL). The resin was then shaken with 50% morpholine in DMA (5 mL) for 10 min, the solution emptied, fresh 50% morpholine in DMA (5 mL) was added and the mixture shaken for another 20 min. The solution was emptied and the resin washed with DMA (5×5 mL). Fmoc-glycine (240 mg; 0.8 mmol), HATU (310 mg; 0.8 mmol) and DIEA (260 μL; 1.6 mmol) were stirred for 15 min in DMA (5 mL) then the solution was added to the resin, the mixture shaken for 2 h at room temperature, emptied and the resin washed with DMA (5×5 mL). The resin was then shaken with 50% morpholine in DMA (5 mL) for 10 min, the solution emptied, fresh 50% morpholine in DMA (5 mL) was added and the mixture shaken for another 20 min. The solution was emptied and the resin washed with DMA (5×5 mL). H (532 mg; 0.80 mmol), HOBt (120 mg; 0.80 mmol), DIC (130 μL; 0.80 mmol), and DIEA (260 μL; 1.6 mmol) and DMA (5 mL) were added to the resin. The mixture was shaken in a flask for 40 h at room temperature, emptied and the resin washed with DMA (5×5 mL), CH₂Cl₂ (5×5 mL) and vacuum dried. The resin was shaken in a flask with Reagent B (25 mL) for 4 h. The resin was filtered and the solution was evaporated under reduced pressure to afford an oily crude that after treatment with Et₂O (20 mL) gave a precipitate. The precipitate was collected by centrifugation and washed with Et₂O (3×20 mL) to give a solid (90 mg) which was analysed by HPLC. An amount of crude (50 mg) was purified by preparative HPLC. The fractions containing the product were lyophilised to give L237 (6 mg; 3.9×10⁻³ mmol) as a white solid. Yield 3.5%.

### Example XI

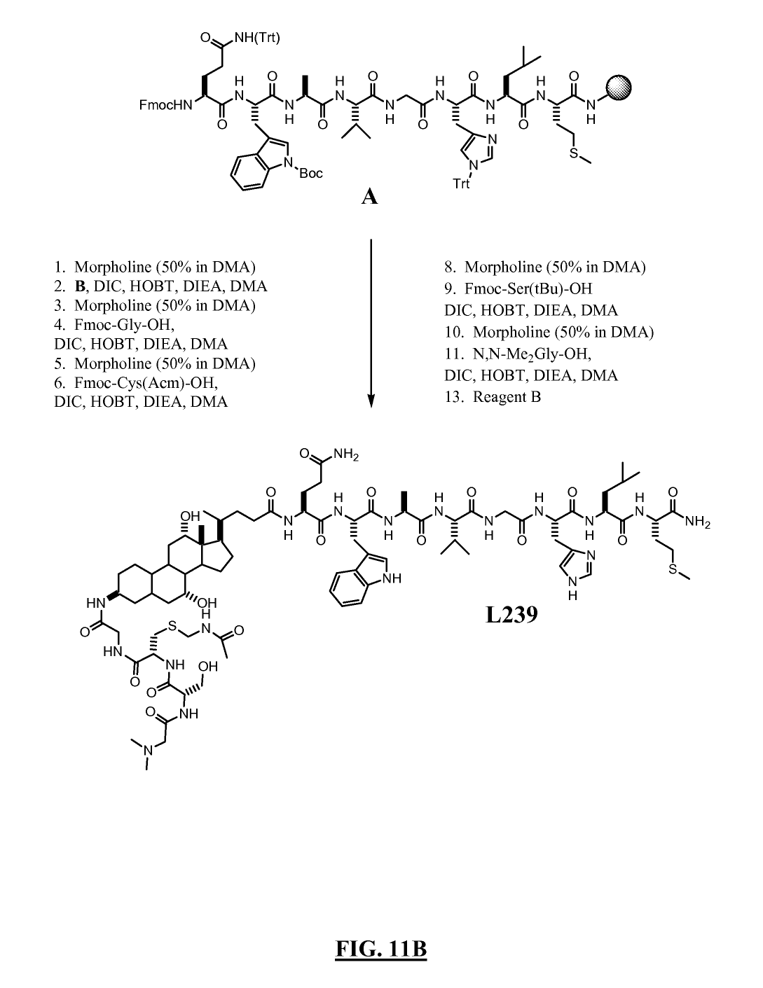

### FIGS. 11A-B

### Synthesis of L238 and L239

Summary: The products were obtained in several steps starting from the octapeptide Gln-Trp-Ala-Val-Gly-His-Leu-Met-NH₂ (BBN[7-14]) (A) on the Rink amide resin. After cleavage and deprotection with Reagent B the crude was purified by preparative HPLC to give L238 and L239. Overall yields: 14 and 9%, respectively.

### A. N,N-Dimethylglycyl-L-seryl-[S-[(acetylamino)methyl]]-L-cysteinyl-glycyl-4-aminobenzoyl-L-glutaminyl-L-tryptophyl-L-alanyl-L-valyl-glycyl-L-histidyl-L-leucyl-L-methioninamide, L238 (FIG. 11A)

Resin A (0.5 g; 0.3 mmol) was shaken in a solid phase peptide synthesis vessel with 50% morpholine in DMA (7 mL) for 10 min, the solution emptied and fresh 50% morpholine in DMA (7 mL) was added. The suspension was stirred for another 20 min then the solution was emptied and the resin washed with DMA (5×7 mL). Fmoc-4-aminobenzoic acid (0.43 g; 1.2 mmol), HOBt (0.18 g; 1.2 mmol), DIC (0.19 mL; 1.2 mmol) and DMA (7 mL) were added to the resin, the mixture shaken for 3 h at room temperature, emptied and the resin washed with DMA (5×7 mL). The resin was then shaken with 50% morpholine in DMA (7 mL) for 10 min, the solution emptied, fresh 50% morpholine in DMA (7 mL) was added and the mixture shaken for another 20 min. The solution was emptied and the resin washed with DMA (5×7 mL). Fmoc-glycine (0.36 g; 1.2 mmol), HATU (0.46 g; 1.2 mmol) and N-ethyldiisopropylamine (0.40 mL; 2.4 mmol) were stirred for 15 min in DMA (7 mL) then the solution was added to the resin, the mixture shaken for 2 h at room temperature, emptied and the resin washed with DMA (5×7 mL). The resin was then shaken with 50% morpholine in DMA (7 mL) for 10 min, the solution emptied, fresh 50% morpholine in DMA (7 mL) was added and the mixture shaken for another 20 min. The solution was emptied and the resin washed with DMA (5×7 mL). N-α-Fmoc-5-acetamidomethyl-L-cysteine (0.50 g; 1.2 mmol), HOBt (0.18 g; 1.2 mmol), DIC (0.19 mL; 1.2 mmol) and DMA (7 mL) were added to the resin, the mixture shaken for 3 h at room temperature, emptied and the resin washed with DMA (5×7 mL). The resin was then shaken with 50% morpholine in DMA (7 mL) for 10 min, the solution emptied, fresh 50% morpholine in DMA (7 mL) was added and the mixture shaken for another 20 min. The solution was emptied and the resin washed with DMA (5×7 mL). N-α-Fmoc-O-t-butyl-L-serine (0.46 g; 1.2 mmol), HOBt (0.18 g; 1.2 mmol), DIC (0.19 mL: 1.2 mmol), and DMA (7 mL) were added to the resin, the mixture was shaken for 3 h at room temperature, emptied and the resin washed with DMA (5×7 mL). The resin was then shaken with 50% morpholine in DMA (7 mL) for 10 min, the solution emptied, fresh 50% morpholine in DMA (7 mL) was added and the mixture shaken for another 20 min.

The solution was emptied and the resin washed with DMA (5×7 mL). N,N-Dimethylglycine (0.12 g; 1.2 mmol), HATU (0.46 g; 1.2 mmol) and N-ethyldiisopropylamine (0.40 mL; 2.4 mmol) were stirred for 15 min in DMA (7 mL) then the solution was added to the resin. The mixture was shaken for 2 h at room temperature, emptied and the resin washed with DMA (5×7 mL), CH₂Cl₂ (5×7 mL) and vacuum dried. The resin was shaken in a flask with Reagent B (25 mL) for 4.5 h. The resin was filtered and the solution was evaporated under reduced pressure to afford an oily crude that after treatment with Et₂O (20 mL) gave a precipitate. The precipitate was collected by centrifugation and washed with Et₂O (3×20 mL) to give a solid (169 mg) which was analysed by HPLC. An amount of crude (60 mg) was purified by preparative HPLC. The fractions containing the product were lyophilised to give L238 (22.0 mg; 0.015 mmol) as a white solid. Yield 14%.

### B. N,N-Dimethylglycyl-L-seryl-[S-[(acetylamino)methyl]]-L-cysteinyl-glycyl-(3β,5β,7α,12α)-3-amino-7,12-dihydroxy-24-oxocholan-24-yl-L-glutaminyl-L-tryptophyl-L-alanyl-L-valyl-glycyl-L-histidyl-L-leucyl-L-methioninamide, L239 (FIG. 11B)

Resin A (0.5 g; 0.3 mmol) was shaken in a solid phase peptide synthesis vessel with 50% morpholine in DMA (7 mL) for 10 min, the solution emptied and fresh 50% morpholine in DMA (7 mL) was added. The suspension was stirred for another 20 min then the solution was emptied and the resin washed with DMA (5×7 mL). (3β,5β,7α,12α)-3-[[(9H-Fluoren-9-ylmethoxy)amino]acetyl]amino-7,12-dihydroxycholan-24-oic acid B (0.82 g; 1.2 mmol) (7), HOBt (0.18 g; 1.2 mmol), DIC (0.19 mL; 1.2 mmol) and DMA (7 mL) were added to the resin, the mixture shaken for 24 h at room temperature, emptied and the resin washed with DMA (5×7 mL). The resin was then shaken with 50% morpholine in DMA (7 mL) for 10 min, the solution emptied, fresh 50% morpholine in DMA (7 mL) was added and the mixture shaken for another 20 min. The solution was emptied and the resin washed with DMA (5×7 mL). N-α-Fmoc-5-acetamidomethyl-L-cysteine (0.50 g; 1.2 mmol), HOBt (0.18 g; 1.2 mmol), DIC (0.19 mL; 1.2 mmol) and DMA (7 mL) were added to the resin, the mixture was shaken for 3 h at room temperature, emptied and the resin washed with DMA (5×7 mL). The resin was then shaken with 50% morpholine in DMA (7 mL) for min, the solution emptied, fresh 50% morpholine in DMA (7 mL) was added and the mixture shaken for another 20 min. The solution was emptied and the resin washed with DMA (5×7 mL). N-α-Fmoc-O-t-butyl-L-serine (0.46 g; 1.2 mmol), HOBt (0.18 g; 1.2 mmol), DIC (0.19 mL: 1.2 mmol), and DMA (7 mL) were added to the resin, the mixture was shaken for 3 h at room temperature, emptied and the resin washed with DMA (5×7 mL). The resin was then shaken with 50% morpholine in DMA (7 mL) for 10 min, the solution emptied, fresh 50% morpholine in DMA (7 mL) was added and the mixture shaken for another 20 min. The solution was emptied and the resin washed with DMA (5×7 mL). N,N-Dimethylglycine (0.12 g; 1.2 mmol), HATU (0.46 g; 1.2 mmol) and N-ethyldiisopropylamine (0.40 mL; 2.4 mmol) were stirred for 15 min in DMA (7 mL) then the solution was added to the resin.

Resin A (0.5 g; 0.3 mmol) was shaken in a solid phase peptide synthesis vessel with 50% morpholine in DMA (7 mL) for 10 min, the solution emptied and fresh 50% morpholine in DMA (7 mL) was added. The suspension was stirred for another 20 min then the solution was emptied and the resin washed with DMA (5×7 mL). (3β,5β,7α,12α)-3-[[(9H-Fluoren-9-ylmethoxy)amino]acetyl]amino-7,12-dihydroxycholan-24-oic acid B (0.82 g; 1.2 mmol) HOBt (0.18 g; 1.2 mmol), DIC (0.19 mL; 1.2 mmol) and DMA (7 mL) were added to the resin, the mixture shaken for 24 h at room temperature, emptied and the resin washed with DMA (5×7 mL). The resin was then shaken with 50% morpholine in DMA (7 mL) for 10 min, the solution emptied, fresh 50% morpholine in DMA (7 mL) was added and the mixture shaken for another 20 min. The solution was emptied and the resin washed with DMA (5×7 mL). N-α-Fmoc-5-acetamidomethyl-L-cysteine (0.50 g; 1.2 mmol), HOBt (0.18 g; 1.2 mmol), DIC (0.19 mL; 1.2 mmol) and DMA (7 mL) were added to the resin, the mixture was shaken for 3 h at room temperature, emptied and the resin washed with DMA (5×7 mL). The resin was then shaken with 50% morpholine in DMA (7 mL) for 10 min, the solution emptied, fresh 50% morpholine in DMA (7 mL) was added and the mixture shaken for another 20 min. The solution was emptied and the resin washed with DMA (5×7 mL). N-α-Fmoc-O-t-butyl-L-serine (0.46 g; 1.2 mmol), HOBt (0.18 g; 1.2 mmol), DIC (0.19 mL: 1.2 mmol), and DMA (7 mL) were added to the resin, the mixture was shaken for 3 h at room temperature, emptied and the resin washed with DMA (5×7 mL). The resin was then shaken with 50% morpholine in DMA (7 mL) for 10 min, the solution emptied, fresh 50% morpholine in DMA (7 mL) was added and the mixture shaken for another 20 min. The solution was emptied and the resin washed with DMA (5×7 mL). N,N-Dimethylglycine (0.12 g; 1.2 mmol), HATU (0.46 g; 1.2 mmol) and N-ethyldiisopropylamine (0.40 mL; 2.4 mmol) were stirred for 15 min in DMA (7 mL) then the solution was added to the resin.

### Example XII

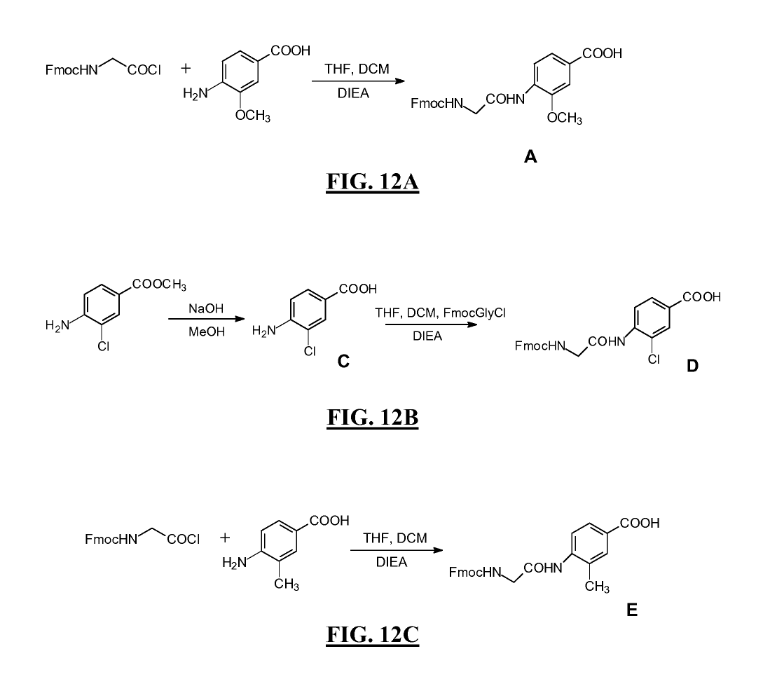

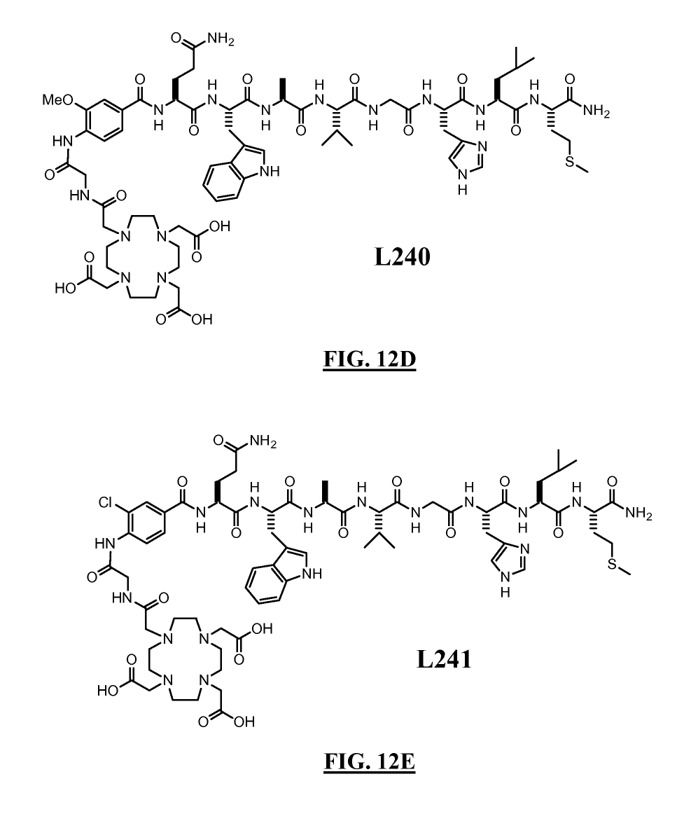

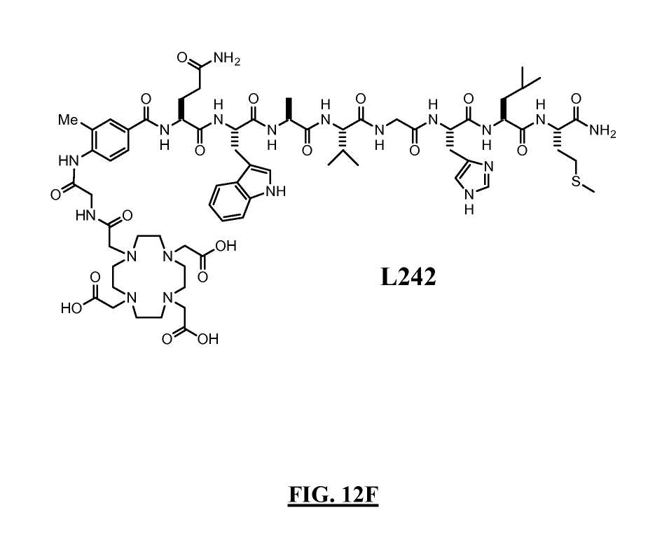

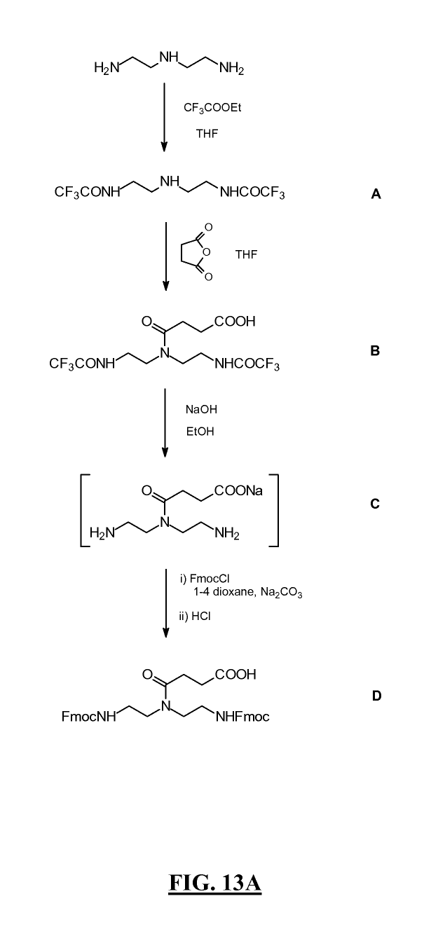

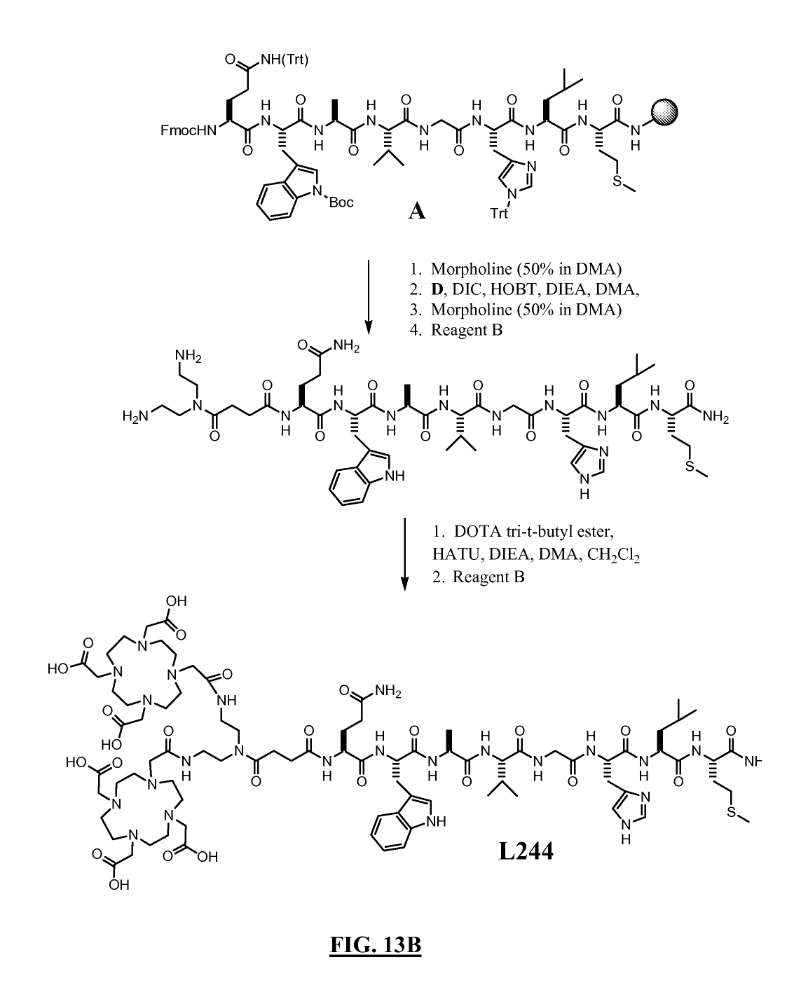

### FIGS. 12A-F

### Synthesis of L240, L241, L242

Summary: The products were obtained in several steps starting from the octapeptide Gln-Trp-Ala-Val-Gly-His-Leu-Met-NH₂ (BBN[7-14]) (A) on the Rink amide resin. After cleavage and deprotection with Reagent B the crudes were purified by preparative HPLC to give L240, L241, and L242. Overall yields: 7.4, 3.2, 1.3% respectively.

### A. 4-[[[(9H-Fluoren-9-ylmethoxy)carbonyl]amino]acetyl]amino-3-methoxybenzoic acid A (FIG. 12A)

A solution of 4-amino-3-methoxybenzoic acid (1.0 g; 5.9 mmol); and N-ethyldiisopropylamine (1.02 mL 5.9 mmol) in THF (20 mL) was added dropwise to a solution of Fmoc-glycylchloride (1.88 g; 5.9 mmol) in CH₂Cl₂/THF 1:1 (20 mL) and stirred at room temperature under N₂. After 3 h the solvent was evaporated under vacuum. The residue was taken up with 0.5 N HCl (50 mL), was stirred for 1 h at 0° C. then filtered and dried. The white solid was suspended in MeOH (30 mL) and stirred for 1 h, then was filtered and suspended in a solution of CHCl₃/hexane 1:4 (75 mL). The suspension was filtered to give compound A as a with solid (1.02 g; 2.28 mmol). Yield 39%.

### B. N-[4-[[[[4,7,10-Tris(carboxymethyl)-1,4,7,10tetraazacyclododec-1-yl]acetyl]glycyl]amino]-3-methoxybenzoyl]-L-glutaminyl-L-tryptophyl-1-alanyl-L-valyl-glycyl-L-histidyl-L-leucyl-L-methioninamide L240

Resin A (0.5 g; 0.3 mmol) was shaken in a solid phase peptide synthesis vessel with 50% morpholine in DMA (7 mL) for 10 min, the solution emptied and fresh 50% morpholine in DMA (7 mL) was added. The suspension was stirred for another 20 min then the solution was emptied and the resin washed with DMA (5×7 mL). 4-[[[(9H-Fluoren-9-ylmethoxy)carbonyl]amino]acetyl]amino-3-methoxybenzoic acid, A (0.50 g; 1.2 mmol), HOBt (0.18 g; 1.2 mmol), DIC (0.19 mL; 1.2 mmol) and DMA (7 mL) were added to the resin, the mixture shaken for 5 h at room temperature, emptied and the resin washed with DMA (5×7 mL). The resin was then shaken with 50% morpholine in DMA (7 mL) for 10 min, the solution emptied, fresh 50% morpholine in DMA (7 mL) was added and the mixture shaken for another 20 min. The solution was emptied and the resin washed with DMA (5×7 mL). 1,4,7,10-Tetraazacyclododecane-1,4,7,10-tetraacetic acid tris(1,1-dimethylethyl) ester adduct with NaCl (0.79 g; 1.2 mmol), HOBt (0.18 g; 1.2 mmol), DIC (0.19 mL: 1.2 mmol), N-ethyldiisopropylamine (0.40 mL; 2.4 mmol) and DMA (7 mL) were added to the resin. The mixture was shaken for 24 h at room temperature, emptied and the resin washed with DMA (5×7 mL), CH₂Cl₂ (5×7 mL) and vacuum dried. The resin was shaken in a flask with Reagent B (25 mL) for 4.5 h. The resin was filtered and the solution was evaporated under reduced pressure to afford an oil crude that after treatment with Et₂O (20 mL) gave a precipitate. The precipitate was collected by centrifugation and washed with Et₂O (5×20 mL) to give a solid (152 mg) which was analysed by HPLC. An amount of crude (52 mg) was purified by preparative HPLC. The fractions containing the product were lyophilized to give L240 (12.0 mg; 7.8×10⁻³ mmol) as a white solid. Yield 7.4%.

### C. 4-amino-3-chlorobenzoic acid C (FIG. 12B)

N NaOH (11 mL; 11 mmol) was added to a solution of methyl 4-amino-3-chlorobenzoate (2 g; 10.8 mmol) in MeOH (20 mL) at 45° C. The reaction mixture was stirred for 5 h at 45° C. and overnight at room temperature. More 1N NaOH was added (5 mL; mmol) and the reaction was stirred at 45° C. for 2 h. After concentration of solvent was added 1N HCl (16 ml). The solid precipitate was filtered and dried to give 4-amino-3-chlorobenzoic acid, C, as a with solid (1.75 g; 10.2 mmol). Yield 94.6%.

### D. 4-[[[(9H-Fluoren-9-ylmethoxy)carbonyl]amino]acetyl]amino-3-chlorobenzoic acid, D (FIG. 12B)

A solution of 4-amino-3-chlorobenzoic acid (1.5 g; 8.75 mmol) and N-ethyldiisopropylamine (1.46 mL 8.75 mmol) in THF (50 mL) was added dropwise to a solution of Fmoc-glycylchloride (2.76 g; 8.75 mmol) in CH₂Cl₂/THF 1:1 (30 mL) and stirred at room temperature under N₂. After 3 h the solvent was evaporated under vacuum. The residue was taken up with 0.5N HCl (50 mL), filtered and dried. The white solid was suspended in MeOH (30 mL) and stirred for 1 h, then was filtered and dried to give 4-[[[(9H-fluoren-9-ylmethoxy)carbonyl]amino]acetyl]amino-3-chlorobenzoic acid (2.95 g; 6.5 mmol). Yield 75%.

### E. N-[4-[[[[4,7,10-Tris(carboxymethyl)-1,4,7,10tetraazacyclododec-1-yl]acetyl]glycyl]amino]3-chlorobenzoyl]L-glutaminyl-L-tryptophyl-1-alanyl-L-valyl-glycyl-L-histidyl-L-leucyl-L-methioninamide, L241 (FIG. 12E)

Resin A (0.5 g; 0.3 mmol) was shaken in a solid phase peptide synthesis vessel with 50% morpholine in DMA (7 mL) for 10 min, the solution emptied and fresh 50% morpholine in DMA (7 mL) was added. The suspension was stirred for another 20 min then the solution was emptied and the resin washed with DMA (5×7 mL). 4-[[[(9H-Fluoren-9-ylmethoxy)carbonyl]amino]acetyl]amino-3-chlorobenzoic acid, D (0.54 g; 1.2 mmol), HOBt (0.18 g; 1.2 mmol), DIC (0.19 mL; 1.2 mmol) and DMA (7 mL) were added to the resin, the mixture shaken for 5 h at room temperature, emptied and the resin washed with DMA (5×7 mL).

The resin was then shaken with 50% morpholine in DMA (7 mL) for 10 min, the solution emptied, fresh 50% morpholine in DMA (7 mL) was added and the mixture shaken for another 20 min. The solution was emptied and the resin washed with DMA (5×7 mL). 1,4,7,10-Tetraazacyclododecane-1,4,7,10-tetraacetic acid tris(1,1-dimethylethyl) ester adduct with NaCl (0.79 g; 1.2 mmol), HOBt (0.18 g; 1.2 mmol), DIC (0.19 mL: 1.2 mmol), N-ethyldiisopropylamine (0.40 mL; 2.4 mmol) and DMA (7 mL) were added to the resin. The mixture was shaken for 40 h at room temperature, emptied and the resin washed with DMA (5×7 mL), CH₂Cl₂ (5×7 mL) and vacuum dried. The resin was shaken in a flask with Reagent B (25 mL) for 4.5 h. The resin was filtered and the solution was evaporated under reduced pressure to afford an oil crude that after treatment with Et₂O (20 mL) gave a precipitate. The precipitate was collected by centrifugation and washed with Et₂O (5×20 mL) to give a solid (151 mg) which was analysed by HPLC. An amount of crude (56 mg) was purified by preparative HPLC. The fractions containing the product were lyophilized to give L241 (5.6 mg; 3.6×10⁻³ mmol) as a white solid. Yield 3.2%.

### F. 4-[[[(9H-Fluoren-9-ylmethoxy)carbonyl]amino]acetyl]amino-3-methylbenzoic acid, E (FIG. 12C)

A solution of 4-amino-3-methylbenzoic acid (0.81 g; 5.35 mmol) and N-ethyldiisopropylamine (0.9 mL 5.35 mmol) in THF (30 mL) was added dropwise to a solution of Fmoc-glycylchloride (1.69 g; 5.35 mmol) in CH₂Cl₂/THF 1:1 (20 mL) and stirred at room temperature under N₂. After 3 h the solvent was evaporated under vacuum. The residue was taken up with HCl 0.5 N (50 mL) and was stirred for 3 h at 0° C., then was filtered and dried. The white solid was suspended in MeOH (50 mL) and stirred for 1 h, then filtered and dried to give Compound E (1.69 g; 3.9 mmol). Yield 73%.

G. N-[4-[[[[4,7,10-Tris(carboxymethyl)-1,4,7,10-tetraazacyclododec-1-yl]acetyl]glycyl]amino]3-methylbenzoyl]L-glutaminyl-L-tryptophyl-L-alanyl-L-valyl-glycyl-L-histidyl-L-leucyl-L-methioninamide L242 (FIG. 12F)

Resin A (0.5 g; 0.3 mmol) was shaken in a solid phase peptide synthesis vessel with 50% morpholine in DMA (7 mL) for 10 min, the solution emptied and fresh 50% morpholine in DMA (7 mL) was added. The suspension was stirred for another 20 min then the solution was emptied and the resin washed with DMA (5×7 mL). 4-[[(9H-Fluoren-9-ylmethoxy)amino]acetyl]amino-3-methylbenzoic acid, E (0.52 g; 1.2 mmol), HOBt (0.18 g; 1.2 mmol), DIC (0.19 mL; 1.2 mmol) and DMA (7 mL) were added to the resin, the mixture shaken for 5 h at room temperature, emptied and the resin washed with DMA (5×7 mL). The resin was then shaken with 50% morpholine in DMA (7 mL) for 10 min, the solution emptied, fresh 50% morpholine in DMA (7 mL) was added and the mixture shaken for another 20 min. The solution was emptied and the resin washed with DMA (5×7 mL). 1,4,7,10-Tetraazacyclododecane-1,4,7,10-tetraacetic acid tris(1,1-dimethylethyl) ester adduct with NaCl (0.76 g; 1.2 mmol), HOBt (0.18 g; 1.2 mmol), DIC (0.19 mL; 1.2 mmol), N-ethyldiisopropylamine (0.40 mL; 2.4 mmol) and DMA (7 mL) were added to the resin.

The mixture was shaken for 40 h at room temperature, emptied and the resin washed with DMA (5×7 mL), CH₂Cl₂ (5×7 mL) and vacuum dried. The resin was shaken in a flask with Reagent B (25 mL) for 4.5 h. The resin was filtered and the solution was evaporated under reduced pressure to afford an oil crude that after treatment with Et₂O (20 mL) gave a precipitate. The precipitate was collected by centrifugation and washed with Et₂O (5×20 mL) to give a solid (134 mg) which was analysed by HPLC. An amount of crude (103 mg) was purified by preparative HPLC. The fractions containing the product were lyophilized to give L242 (4.5 mg; 2.9×10⁻³ mmol) as a white solid. Yield 1.3%.

### Example XIII

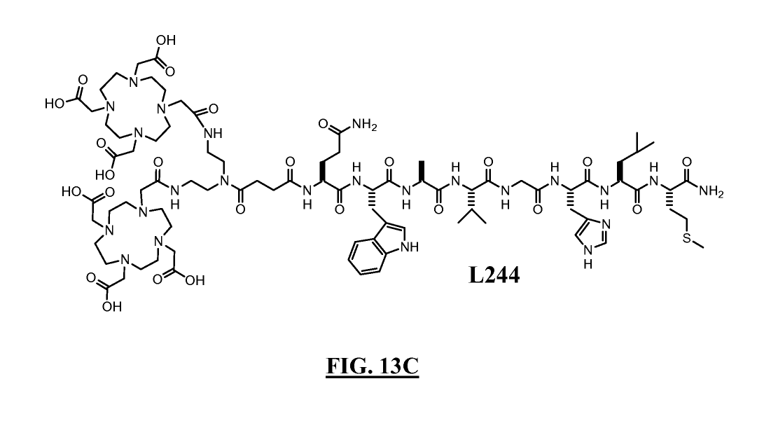

### FIGS. 13A-C

### Synthesis of L244

Summary: The product was obtained in several steps starting from the octapeptide Gln-Trp-Ala-Val-Gly-His-Leu-Met-NH₂ (BBN[7-14]) on the Rink amide resin (A). The final coupling step with DOTA tri-t-butyl ester was done in solution phase after cleavage and deprotection with Reagent B of Linker-BBN [7-14]. The crude was purified by preparative HPLC to give L244. Overall yield: 0.4%.

### A. N,N′-(Iminodi-2,1-ethanediyl)bis[2,2,2-trifluoroacetamide], A (FIG. 13A)

Trifluoroacetic acid ethyl ester (50 g; 0.35 mol) was dropped into a solution of diethylenetriamine (18 g; 0.175 mol) in THF (180 mL) at 0° C. in 1 h. After 20 h at room temperature, the mixture was evaporated to an oily residue (54 g). The oil was crystallized from Et₂O (50 mL), filtered, washed with cooled Et₂O (2×30 mL) and dried to obtain A as a white solid (46 g; 0.156 mol). Yield 89%.

### B. 4-[N,N′-Bis[2-(trifluoroacetyl)aminoethyl]amino]-4-oxobutanoic acid, B (FIG. 13A)

Succinic anhydride (0.34 g; 3.4 mmol) was added in a solution of A (1 g; 3.4 mmol) in THF (5 mL) at room temperature. After 28 h the crude was concentrated to residue (1.59 g), washed with EtOAc (2×10 mL) and 1 N HCl (2×15 mL). The organic layer was dried on Na₂SO₄, filtered and evaporated to give an oily residue (1.3 g) that was purified by flash chromatography (5) to afford B as an oil (0.85 g; 2.15 mmol). Yield 63%.

### C. 4-[N,N′-Bis[2-[(9-H-fluoren-9-ylmethoxy)carbonyl]aminoethyl]amino]-4-oxobutanoic acid, D (FIG. 13A)

Succinic anhydride (2 g; 20 mmol) was added in a solution of A (5 g; 16.94 mmol) in THF (25 mL) at room temperature. After 28 h the crude was concentrated to residue (7 g), washed in ethyl acetate (100 mL) and in 1 N HCl (2×50 mL). The organic layer was dried on Na₂SO₄, filtered and evaporated to give crude B as an oily residue (6.53 g). 2 N NaOH (25 mL) was added to suspension of crude B (5 g) in EtOH (35 mL) obtaining a complete solution after 1 h at room temperature. After 20 h the solvent was evaporated to obtain C as an oil (8.48 g). A solution of 9-fluorenylmethyl chloroformate (6.54 g, 25.3 mmol) in 1,4-dioxane (30 mL), was dropped in the solution of C in 10% aq. Na₂CO₃ (30 mL) in 1 h at 0° C. After 20 h at r.t. a gelatinous suspension was obtained and filtered to give a white solid (3.5 g) and a yellow solution. The solution was evaporated and the remaining aqueous solution was diluted in H₂OO (150 mL) and extracted with EtOAc (70 mL). Fresh EtOAc (200 mL) was added to aqueous phase, obtaining a suspension which was cooled to 0° C. and acidified to pH 2 with conc. HCl. The organic layer was washed with H₂O (5×200 mL) until neutral pH, then dried to give a glassy solid (6.16 g). The compound was suspended in boiling n-Hexane (60 mL) for 1 h, filtered to give D as a white solid (5.53 g, 8.54 mmol). Overall yield 50%.

### D. N-[4-[[4-[Bis[2-[[[4,7,10-tris(carboxymethyl)-1,4,7,10-tetraazacyclododec-1-yl]acetyl]amino]ethyl]amino-1,4-dioxobutyl]amino]benzoyl]-L-glutaminyl-L-tryptophyl-L-alanyl-L-valyl-glycyl-L-histidyl-L-leucyl-L-methioninamide, L244 (FIG. 13B)

Resin A (0.5 g; 0.3 mmol) was shaken in a solid phase peptide synthesis vessel with 50% morpholine in DMA (7 mL) for 10 min, the solution emptied and fresh 50% morpholine in DMA (7 mL) was added. The suspension was stirred for another 20 min then the solution was emptied and the resin washed with DMA (5×7 mL). 4-[N,N′-Bis[2-[(9-H-fluoren-9-ylmethoxy) carbonyl]aminoethyl]amino]-4-oxo butanoic acid (777.3 mg; 1.2 mmol), HOBt (184 mg; 1.2 mmol), DIC (187 μL; 1.2 mmol) and DMA (7 mL) were added to the resin, the mixture shaken for 40 h at room temperature, emptied and the resin washed with DMA (5×7 mL). The resin was then shaken with 50% morpholine in DMA (7 mL) for min, the solution emptied, fresh 50% morpholine in DMA (7 mL) was added and the mixture shaken for 20 min. The solution was emptied and the resin washed with DMA (2×7 mL) and with CH₂Cl₂ (5×7 mL) then it was shaken in a flask with Reagent B (25 mL) for 4.5 h. The resin was filtered and the solution was evaporated under reduced pressure to afford an oily crude that after treatment with Et₂O (20 mL) gave a precipitate. The precipitate was collected by centrifugation and washed with Et₂O (5×20 mL) to give F as a white solid (140 mg). DOTA tri-t-butyl ester (112 mg; 0.178 mmol) HATU (70 mg; 0.178 mmol) and DIEA (60 μL; 0.356 mmol) were added to a solution of F (50 mg; 0.0445 mmol) in DMA (3 mL) and CH₂Cl₂ (2 mL) and stirred for 24 h at room temperature. The crude was evaporated to reduced volume (1 mL) and shaken with Reagent B (25 mL) for 4.5 h. After evaporation of the solvent, the residue was treated with Et₂O (20 mL) to give a precipitate. The precipitate was collected by centrifugation and washed with Et₂O (5×20 mL) to afford a beige solid (132 mg) that was analyzed by HPLC. An amount of crude (100 mg) was purified by preparative HPLC. The fractions containing the product were lyophilized to give L244 (FIG. 13C) (3.5 mg; 1.84×10⁻³ mmol) as a white solid. Yield 0.8%.

### General Experimentals for Examples XIV

### Example XLII

### L201-L228

### A. Manual Couplings

6.0 equivalents of the appropriately protected amino acid was treated with 6.0 equivalents each of HOBt and DIC and activated outside the reaction vessel. This activated carboxylic acid in NMP was then transferred to the resin containing the amine and the reaction was carried out for 4-6 h and then the resin was drained and washed.

### B. Special coupling of Fmoc-Gly-OH to 4-aminobenzoic acid and aminobiphenylcarboxylic acid amides

Fmoc-Gly-OH (10.0 equiv.) was treated with HATU (10.0 equiv.) and DIEA (20.0 equiv.) in NMP (10 mL of NMP was used for one gram of the amino acid by weight) and the solution was stirred for 10-15 min at RT before transferring to the vessel containing the amine loaded resin. The volume of the solution was made to 15.0 ml for every gram of the resin. The coupling was continued for 20 h at RT and the resin was drained of all the reactants. This procedure was repeated one more time and then washed with NMP before moving on to the next step.

### C. Preparation of D03A Monoamide

8.0 equivalents of DOTA mono acid was dissolved in NMP and treated with 8.0 equivalents of HBTU and 16.0 equivalents of DIEA. This solution was stirred for 15 min at RT and then transferred to the amine on the resin and the coupling was continued for 24 h at RT. The resin was then drained, washed and then the peptide was cleaved and purified.

### D. Cleavage of the Crude Peptides from the Resin and Purification

The resin was suspended in Reagent B (15.0 ml/g) and shaken for 4 h at RT. The resin was then drained and washed with 2×5 mL of Reagent B again and combined with the previous filtrate. The filtrate was then concentrated under reduced pressure to a paste/liquid at RT and triturated with 25.0 mL of anhydrous ether (for every gram of the resin used). The suspension was then centrifuged and the ether layer was decanted. This procedure was repeated two more times and the colorless precipitate after ether wash was purified by preparative HPLC.

### Example XIV

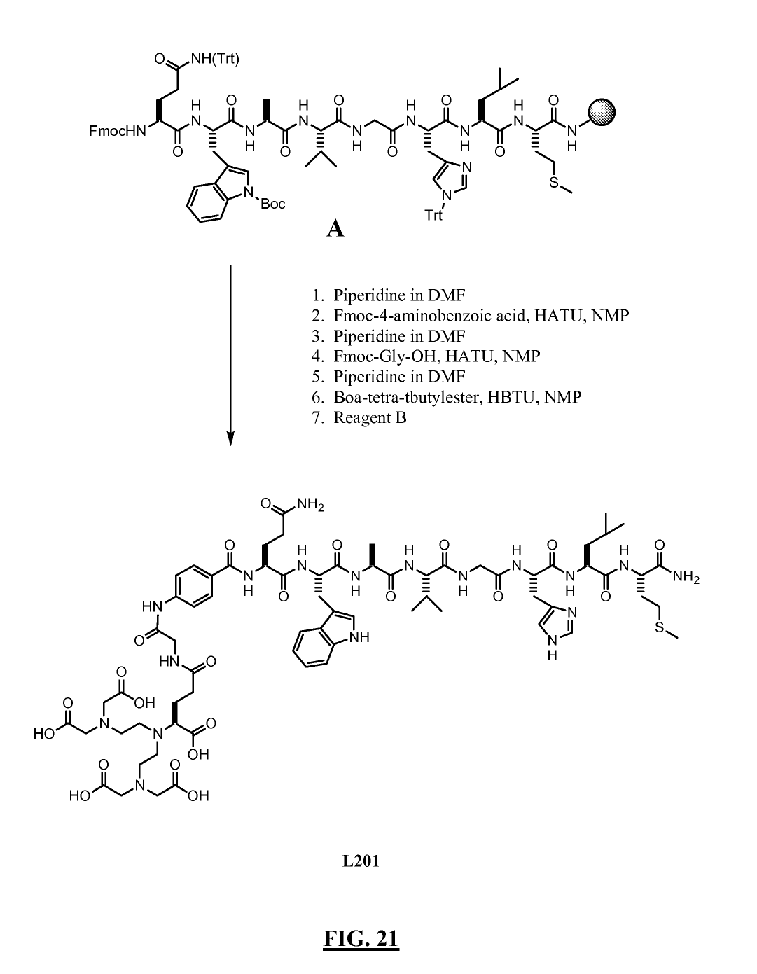

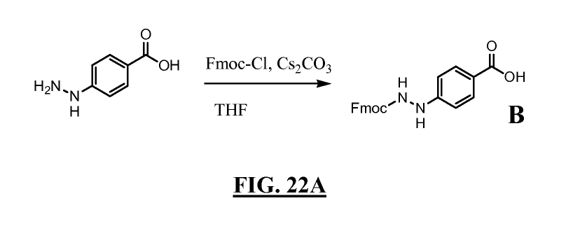

### FIG. 21

### Synthesis of L201

0.5 g of the Fmoc-Q(Trt)-W(Boc)-A-V-G-H(Trt)-M-Resin (0.4 mmol/g, 0.5 g, 0.2 mmol) (Resin A) was used. The rest of the amino acid units were added as described in the general procedure to prepare (1R)-1-(Bis{2-[bis(carboxymethyl)amino]ethyl}amino)propane-3-carboxylic acid-1-carboxyl-glycyl-4-aminobenzoyl-L-glutaminyl-L-tryptophyl-L-alanyl-L-valyl-glycyl-L-histidyl-L-leucyl-L-methioninamide (L201), Yield: 17.0 mg (5.4%)

### Example XV

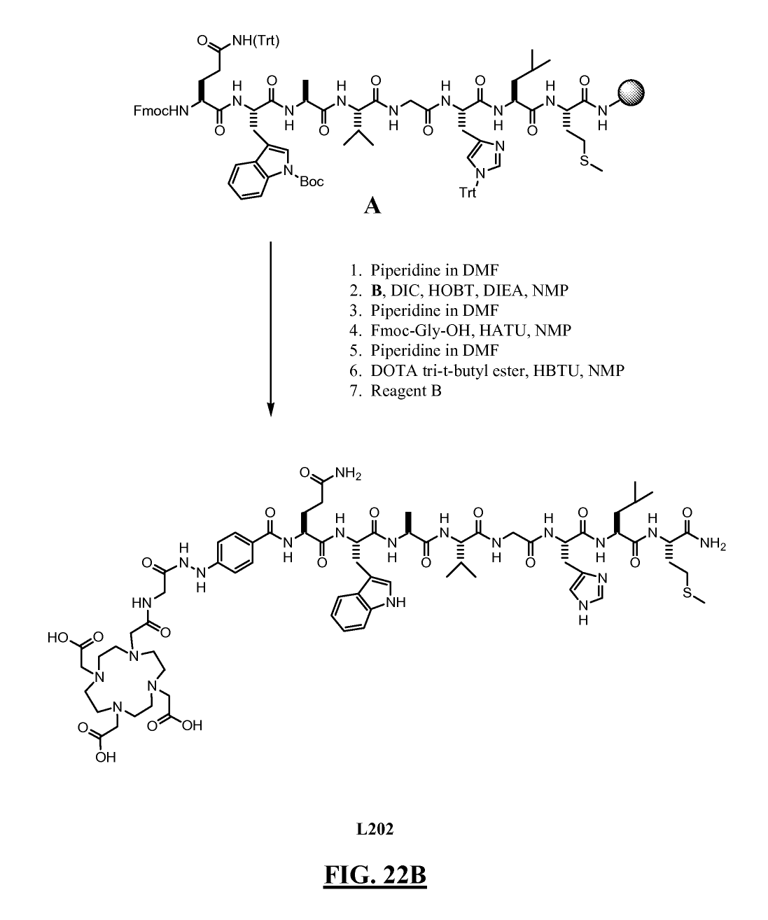

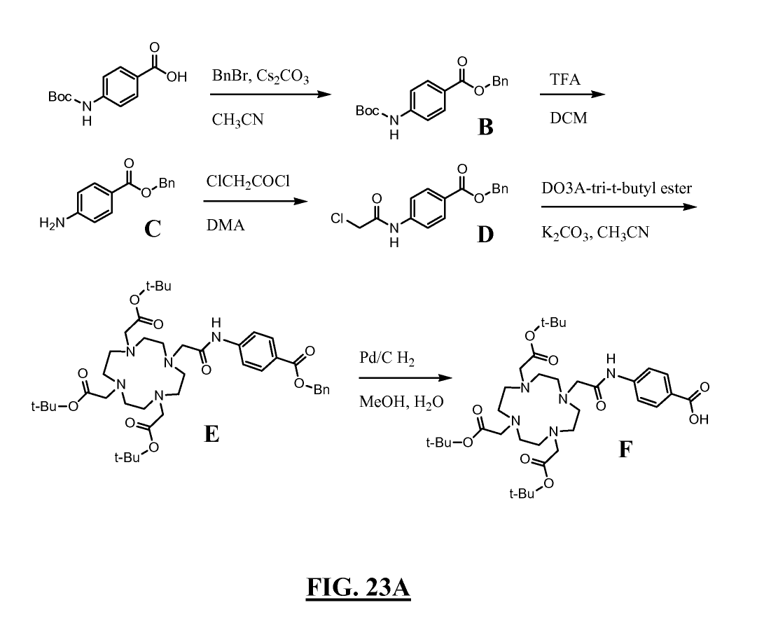

### FIGS. 22A and 22B

### Synthesis of L202

### A. 4-Fmoc-hydrazinobenzoic acid (FIG. 22A)

A suspension of 4-hydrazinobenzoic acid (5.0 g, 32.9 mmol) in water (100 ml) was treated with cesium carbonate (21.5 g, 66.0 mmol). Fmoc-Cl (9.1 g, 35.0 mmol) in THF (25 mL) was added dropwise to the above solution with stirring over a period of 1 h. The solution was stirred for 4 h more after the addition and the reaction mixture was concentrated to about 75 mL and extracted with ether (2×100 mL). The ether layer was discarded and the aqueous layer was acidified with 2N HCl. The separated solid was filtered, washed with water (5×100 mL) and then recrystallized from acetonitrile to yield the product (compound B) as a colorless solid. Yield: 11.0 g (89%). ¹H NMR (DMSO-d₆): δ 4.5 (m, 1H, Ar—CH₂—CH), 4.45 (m, 2H, Ar—CH₂), 6.6 (bs, 1H, Ar—H), 7.4-7.9 (m, 9, Ar—H and Ar—CH₂), 8.3 (s, 2H, Ar—H), 9.6 (s, 2H, Ar—H). M.S.—m/z 373.2 [M−H]

0.5 g of the Fmoc-Q(Trt)-W(Boc)-A-V-G-H(Trt)-M-Resin (0.4 mmol/g, 0.5 g, 0.2 mmol) (Resin A) was used. The amino acid units were added as described in the general procedure, including Compound B to prepare N-[(3β,5β,12α)-3-[[[[[4,7,10-Tris(carboxymethyl)-1,4,7,10-tetraazacyclododec-1-yl]acetyl]amino]acetyl]amino]-4-hydrazinobenzoyl-L-glutaminyl-L-tryptophyl-L-alanyl-L-valyl-glycyl-L-histidyl-L-leucyl-L-methioninamide (L202) (FIG. 22B), Yield: 25.0 mg (8.3%)

### Example XVI

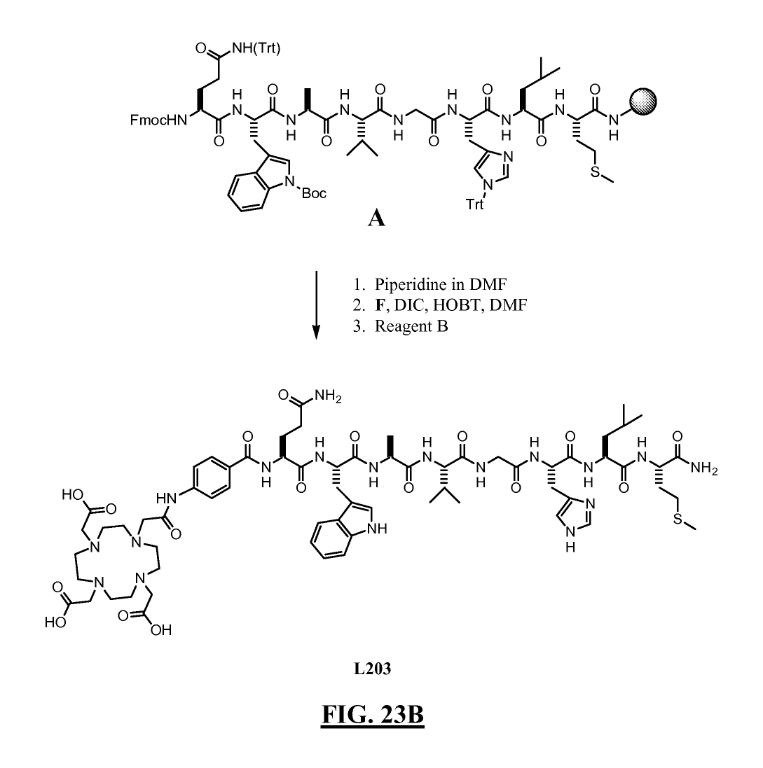

### FIGS. 23A and 23B

### Synthesis of L203

### A. Preparation of 4-Boc-aminobenzyl benzoate Compound B (FIG. 23A)

A suspension of 4-boc-aminobenzoic acid (0.95 g, 4.0 mmol) in dry acetonitrile (10.0 mL) was treated with powdered cesium carbonate (1.3 g, 4.0 mmol) and stirred vigorously under nitrogen. Benzyl bromide (0.75 g, 4.4 mmol) was added and the reaction mixture was refluxed for 20 h under nitrogen. The reaction mixture was then poured into ice cold water (200 mL) and the solid separated was filtered and washed with water (5×50 mL). The crude material was then recrystallized from aqueous methanol to yield the product as a colorless solid (Compound B). Yield: 0.8 g (61%). ¹H NMR (CDCl₃): δ 1.5 (s, 9H, Tertiary methyls), 5.4 (s, 2H, Ar—CH₂), 7.4 (m, 7H, Ar—H) and 8.0 (m, 2H, Ar—H). M.S.—m/z 326.1 [M+H].

### B. 4-Aminobenzyl benzoate Compound C (FIG. 23A)

4-Boc-aminobenzyl benzoate (0.8 g, 2.5 mmol) was dissolved in DCM (20 mL) containing TFA (25% by volume) and stirred for 2 h at RT. The reaction mixture was poured into 100.0 g of crushed ice and neutralized with saturated sodium bicarbonate solution until the pH reached about 8.5. The organic layer was separated and the aqueous layer was extracted with DCM (3×20 mL) and all the organic layers were combined. The DCM layer was then washed with 1×50 mL of saturated sodium bicarbonate, water (2×50 mL) and dried (sodium sulfate). Removal of the solvent yielded a colorless solid (Compound C) that was taken to the next step without further purification. Yield: 0.51 g (91%). ¹H NMR (CDCl₃): δ 5.3 (s, 2H, Ar—CH₂), 6.6 (d, 2H, Ar—H, j=1.0 Hz), 7.4 (m, 5H, Ar—H, J=1.0 Hz) and 7.9 (d, 2H, Ar—H, J=1.0 Hz).

### C. 4-(2-Chloroacetyl)aminobenzyl benzoate Compound D (FIG. 23A)

The amine (0.51 g, 2.2 mmol) was dissolved in dry dimethylacetamide (5.0 mL) and cooled in ice. Chloroacetyl chloride (0.28 g, 2.5 mmol) was added dropwise via a syringe and the solution was allowed to come to RT and stirred for 2 h. An additional, 2.5 mmol of chloroacetyl chloride was added and stirring was continued for 2 h more. The reaction mixture was then poured into ice cold water (100 mL). The precipitated solid was filtered and washed with water and then recrystallized from hexane/ether to yield a colorless solid (Compound D). Yield: 0.38 g (56%). ¹H NMR (CDCl₃): δ 4.25 (s, 2H, CH₂—Cl), 5.4 (s, 2H, Ar—H), 7.4 (m, 5H, Ar—H), 7.6 (d, 2H, Ar—H), 8.2 (d, 2H, Ar—H) and 8.4 (s, 1H, —CONH).

tert-Butyl 2-{1,4,7,10-tetraaza-7,10-bis{[(tert-butyl)oxycarbonyl]methyl}-4-[(N-{4-[benzyloxycarbonyl]phenyl}carbamoyl]cyclododecyl}acetate, Compound E (FIG. 23A):

DO3A-tri-t-butyl ester.HCl (5.24 g, 9.5 mmol) was suspended in 30.0 mL of dry acetonitrile and anhydrous potassium carbonate (2.76 g, 20 mmol) was added and stirred for 30 min. The chloroacetamide D (2.8 g, 9.2 mmol) in dry acetonitrile (20.0 mL) was then added dropwise to the above mixture for 10 min. The reaction mixture was then stirred overnight. The solution was filtered and then concentrated under reduced pressure to a paste. The paste was dissolved in about 200.0 mL of water and extracted with 5×50 mL of ethyl acetate. The combined organic layer was washed with water (2×100 mL) and dried (sodium sulfate). The solution was filtered and evaporated under reduced pressure to a paste and the paste was chromatographed over flash silica gel (600.0 g). Elution with 5% methanol in DCM eluted the product. All the fractions that were homogeneous on TLC were pooled and evaporated to yield a colorless gum. The gum was recrystallized from isopropylether and DCM to prepare Compound E. Yield: 4.1 g (55%). ¹H NMR (CDCl₃): δ 1.5 (s, 27H, methyls), 2.0-3.75 (m, 24H, NCH₂s), 5.25 (d, 2H, Ar—CH₂), 7.3 (m, 5H, Ar—H), 7.8 (d, 2H, Ar—H) and 7.95 (d, 2H, Ar—H). M.S.—m/z 804.3 [M+H].

### D. Reduction of the Above Acid E to Prepare Compound F, (FIG. 23A)

The benzyl ester E from above (1.0 g, 1.24 mmol) was dissolved in methanol-water mixture (10.0 mL, 95:5) and palladium on carbon was added (10%, 0.2 g). The solution was then hydrogenated using a Parr apparatus at 50.0 psi for 8 h. The solution was filtered off the catalyst and then concentrated under reduced pressure to yield a colorless fluffy solid F. It was not purified further and was taken to the next step immediately. MS: m/z 714.3 [M+Na].

### E. Preparation of L203 (FIG. 23B)

The above acid F was coupled to the amine on the resin [H-Q(Trt)-W(Boc)-A-V-G-H(Trt)-L-M-Resin]Resin A and F from above using standard coupling procedures described above. 0.5 g (0.2 mmol) of the resin yielded 31.5 mg of the final purified peptide (10.9%) N-[(3β,5β,12α)-3-[[[4,7,10-Tris(carboxymethyl)-1,4,7,10-tetraazacyclododec-1-yl]acetyl]amino]-4-aminobenzoyl-L-glutaminyl-L-tryptophyl-L-alanyl-L-valyl-glycyl-L-histidyl-L-leucyl-L-methioninamide (L203) (FIG. 23B).

### Example XVII

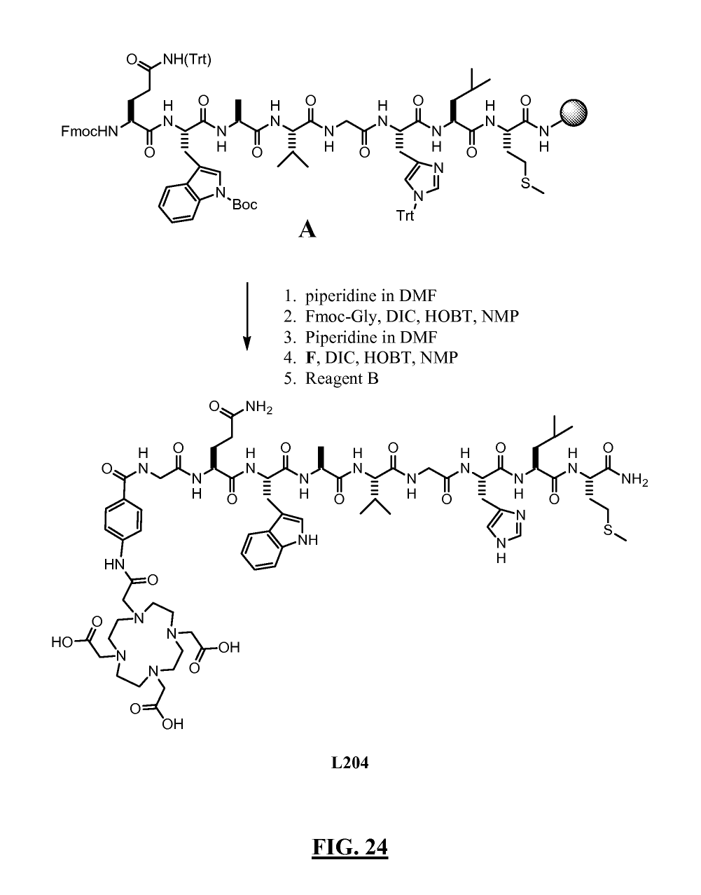

### FIG. 24

### Synthesis of L204

Fmoc-Q(Trt)-W(Boc)-A-V-G-H(Trt)-L-M-resin (0.5 g, 0.2 mmol) (Resin A) was used. Fmoc-Gly-OH was loaded first followed by F from the above procedure (FIG. 23A) employing standard coupling conditions. Yield: 24.5 mg (8.16%) of N-[(3β,5β,12α)-3-[[[4,7,10-Tris(carboxymethyl)-1,4,7,10-tetraazacyclododec-1-yl]acetyl]amino]-4-aminobenzoyl-glycyl-L-glutaminyl-L-tryptophyl-L-alanyl-L-valyl-glycyl-L-histidyl-L-leucyl-L-methioninamide (L204) (FIG. 24).

### Example XVIII

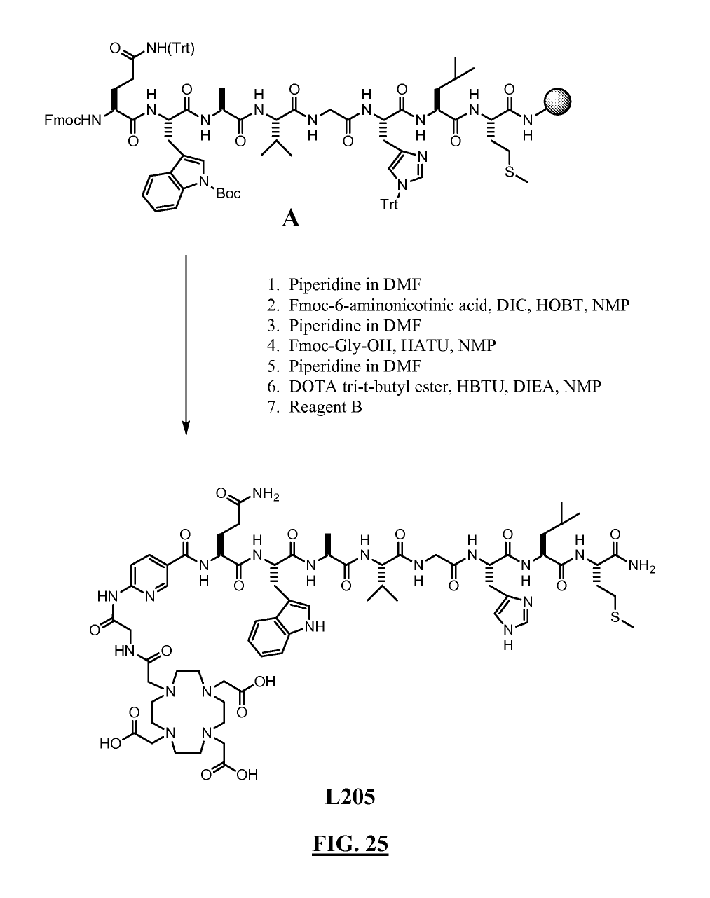

### FIG. 25

### Synthesis of L205

Fmoc-6-aminonicotinic acid¹ was prepared as described in the literature (“Synthesis of diacylhydrazine compounds for therapeutic use”. Hoelzemann, G.; Goodman, S. (Merck Patent G.m.b.H., Germany). Ger.Offen. 2000, 16 pp. CODEN: GWXXBX DE 19831710 A1 20000120) and coupled with preloaded Fmoc-Q(Trt)-W(Boc)-A-V-G-H(Trt)-L-M-resin (0.5 g, 0.2 mmol) Resin A, followed by the other amino groups as above to prepare N-[(3β,5β,12α)-3-[[[4,7,10-Tris(carboxymethyl)-1,4,7,10-tetraazacyclododec-1-yl]acetyl]amino]-4-aminobenzoyl-glycyl-L-glutaminyl-L-tryptophyl-L-alanyl-L-valyl-glycyl-L-histidyl-L-leucyl-L-methioninamide (L205) Yield: 1.28 mg (0.4%).

### Example XIX

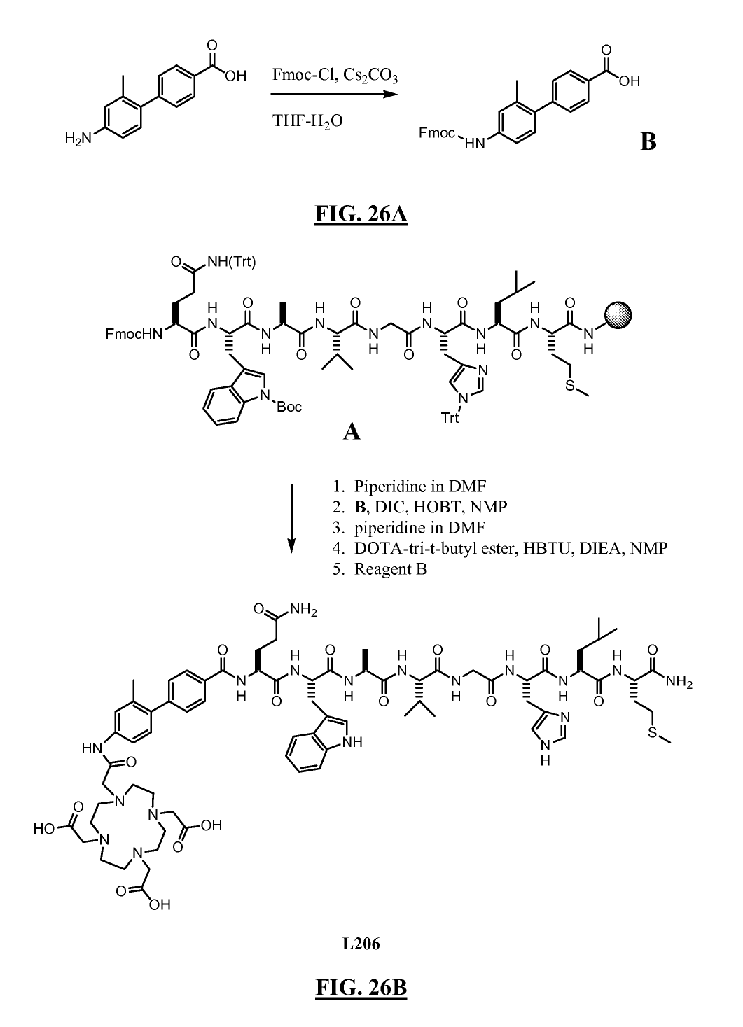

### FIGS. 26A and 26B

### Synthesis of L206

### A. 4′-Fmoc-amino-3′-methylbiphenyl-4-carboxylic acid B

The amino acid (0.41 g, 1.8 mmol) was dissolved in a solution of cesium carbonate (0.98 g, 3.0 mmol) in 10.0 mL of water. See “Rational Design of Diflunisal Analogues with Reduced Affinity for Human Serum Albumin “Mao, H. et al J. Am. Chem. Soc., 2001, 123(43), 10429-10435. This solution was cooled in an ice bath and a solution of Fmoc-Cl (0.52 g, 2.0 mmol) in THF (10.0 mL) was added dropwise with vigorous stirring. After the addition, the reaction mixture was stirred at RT for 20 h. The solution was then acidified with 2N HCl. The precipitated solid was filtered and washed with water (3×20 mL) and air dried. The crude solid was then recrystallized from acetonitrile to yield a colorless fluffy solid B (FIG. 26A). Yield: 0.66 g (75%). ¹H NMR (DMSO-d₆): δ 2.2 (s, Ar-Me), 4.25 (t, 1H, Ar—CH, j=5 Hz), 4.5 (d, 2H, O—CH₂, j=5.0 Hz), 7.1 (bs, 1H, CONH), 7.4-8.0 (m, 8H, Ar—H) and 9.75 (bs, 1H, —COOH). M. S.: m/z 472.0 [M−H].

The acid B from above was coupled to Fmoc-Q(Trt)-W(Boc)-A-V-G-H(Trt)-L-M-resin (0.2 g, 0.08 mmol) resin A with the standard coupling conditions. Additional groups were added as above to prepare N-[(3β,5β,12α)-3-[[[[[4,7,10-Tris(carboxymethyl)-1,4,7,10-tetraazacyclododec-1-yl]acetyl]amino]acetyl]amino]-[4′-Amino-2′-methyl biphenyl-4-carboxyl]-L-glutaminyl-L-tryptophyl-L-alanyl-L-valyl-glycyl-L-histidyl-L-leucyl-L-methioninamide (L206). Yield: 30.5 mg (24%).

### Example XX

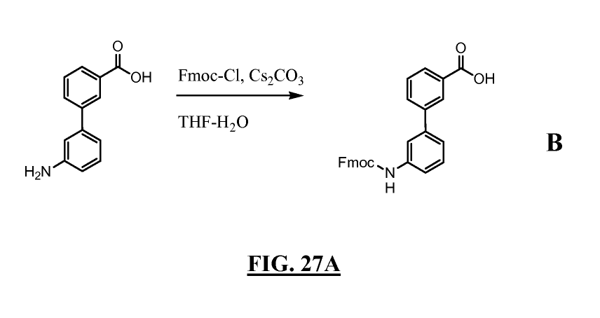

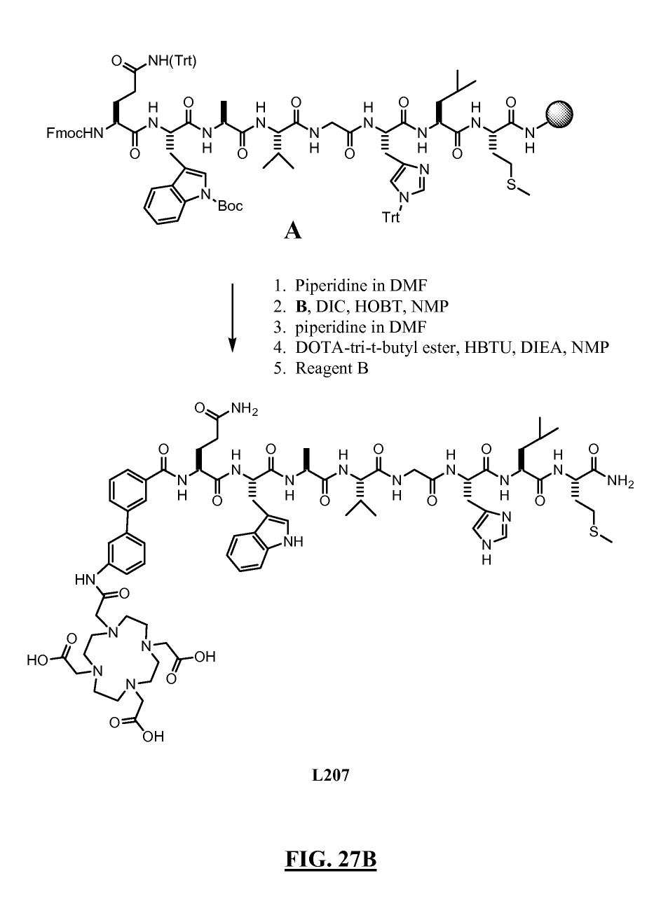

### FIGS. 27A-B

### Synthesis of L207

3′-Fmoc-amino-biphenyl-3-carboxylic acid was prepared from the corresponding amine using the procedure described above. See “Synthesis of 3′-methyl-4-′-nitrobiphenylcarboxylic acids by the reaction of 3-methyl-4-nitrobenzenenediazonium acetate with methyl benzoate”, Boyland, E. and Gorrod, J., J. Chem. Soc., Abstracts (1962), 2209-11. 0.7G of the amine yielded 0.81 g of the Fmoc-derivative (58%) (Compound B, FIG. 27A). ¹H NMR (DMSO-d₆): δ 4.3 (t, 1H, Ar—CH), 4.5 (d, 2H, O—CH₂), 7.25-8.25 (m, 16H, Ar—H) and 9.9 (s, 1H, —COOH). M.S.—m/z 434 [M−H]

Fmoc-Q(Trt)-W(Boc)-A-V-G-H(Trt)-L-M-resin (0.2 g, 0.08 mmol) resin A was coupled to the above acid B and additional groups as above (FIG. 27B). 29.0 mg of N-[(3β,5β,12α)-3-[[[[[4,7,10-Tris(carboxymethyl)-1,4,7,10-tetraazacyclododec-1-yl]acetyl]amino]acetyl]amino]-[3′-amino-biphenyl-3-carboxyl]-L-glutaminyl-L-tryptophyl-L-alanyl-L-valyl-glycyl-L-histidyl-L-leucyl-L-methioninamide (L207) was prepared (23%).

### Example XXI

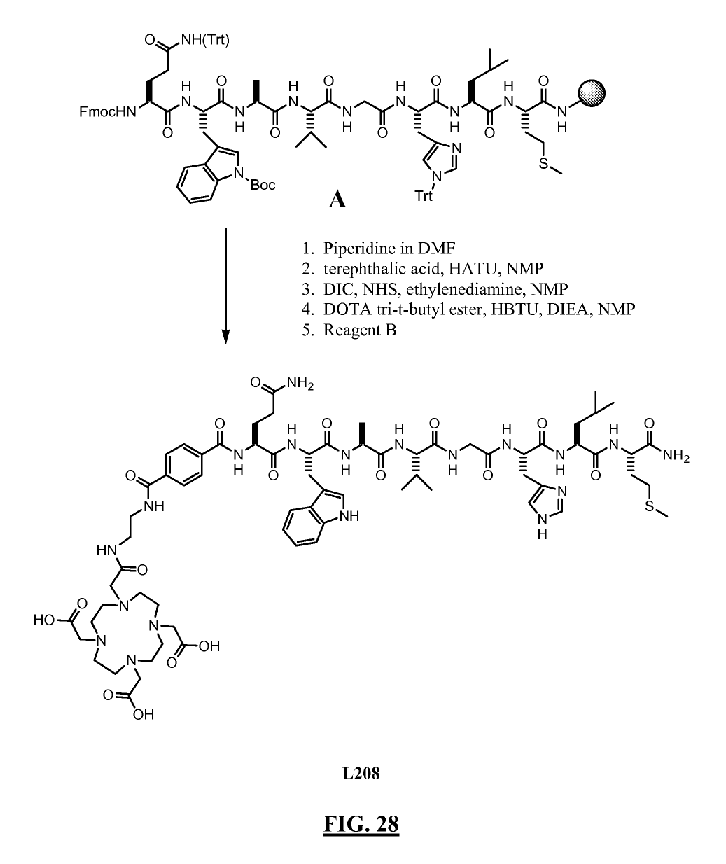

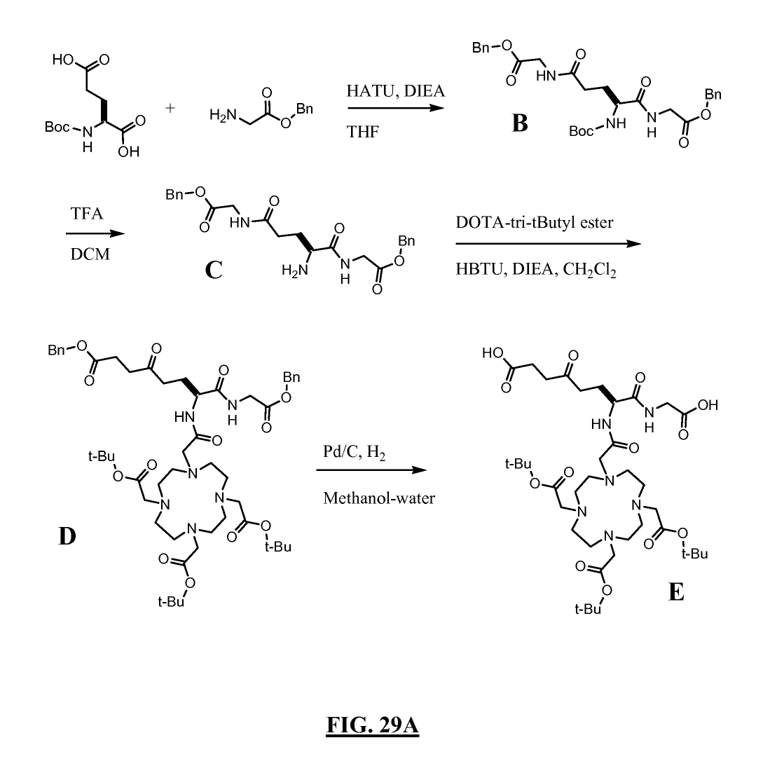

### FIG. 28

### Synthesis of L208

Fmoc-Q(Trt)-W(Boc)-A-V-G-H(Trt)-L-M-resin (0.2 g, 0.08 mmol) A was deblocked and coupled to terephthalic acid employing HATU as the coupling agent. The resulting acid on the resin was activated with DIC and NHS and then coupled to ethylenediamine. DOTA-mono acid was finally coupled to the amine on the resin. N-[(3β,5β,12α)-3-[[[[[4,7,10-Tris(carboxymethyl)-1,4,7,10-tetraazacyclododec-1-yl]acetyl]amino]acetyl]amino]-[1,2-d]aminoethyl-terephthalyl]-L-glutaminyl-L-tryptophyl-L-alanyl-L-valyl-glycyl-L-histidyl-L-leucyl-L-methioninamide (L208) was prepared for a yield of 17.5 mg (14%)

### Example XXII

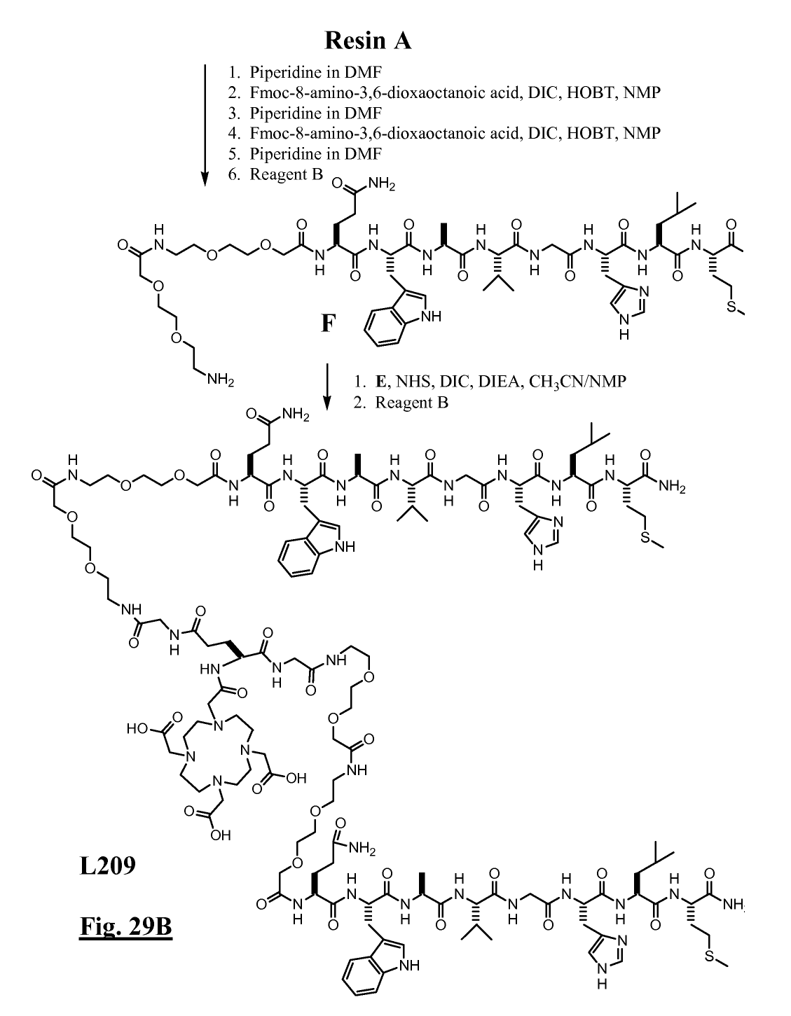

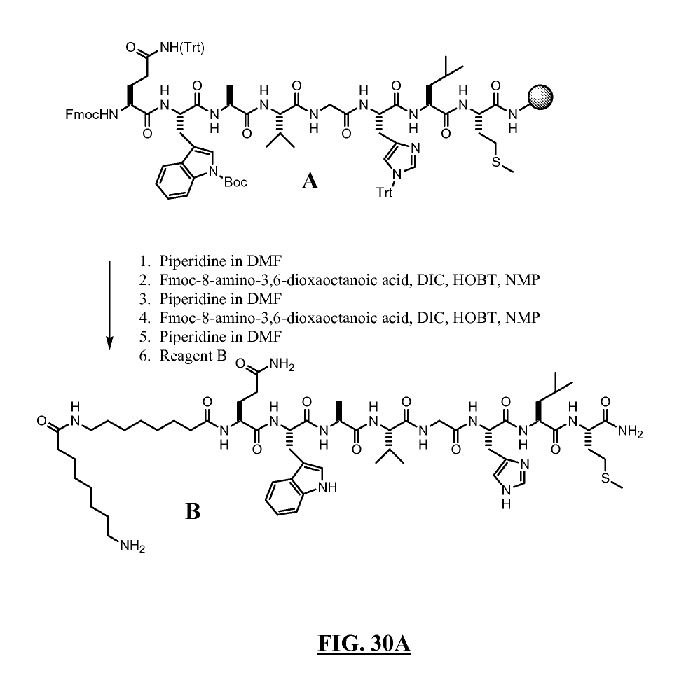

### FIGS. 29A-B

### Synthesis of L209

A. Boc-Glu(G-OBn)-G-OBn:

Boc-Glutamic acid (5.0 g, 20.2 mol) was dissolved in THF (50.0 mL) and cooled to 0° C. in an ice bath. HATU (15.61 g, 41.0 mmol) was added followed by DIEA (6.5 g, 50.0 mmol). The reaction mixture was stirred at 0° C. for 30 min. Benzyl ester of glycine [8.45 g, 50 mmol, generated from neutralizing benzyl glycine hydrochloride with sodium carbonate and by extraction with DCM and solvent removal] was added in THF (25.0 mL). The reaction mixture was allowed to come to RT and stirred for 20 h at RT. All the volatiles were removed under reduced pressure. The residue was treated with saturated sodium carbonate solution (100 mL) and extracted with ethyl acetate (3×100 mL). The organic layers were combined and washed with 1N HCl (2×100 mL) and water (2×100 mL) and dried (sodium sulfate). The solution was filtered and solvent was removed under reduced pressure to yield a paste that was chromatographed over flash silica gel (500.0 g). Elution with 2% methanol in DCM yielded the product as a colorless paste (Compound B, FIG. 29A). Yield: 8.5 g (74.5%). ¹H NMR (CDCl₃): δ 1.4 (s, 9H, —CH₃s), 2.0-2.5 (m, 4H, —CH—CH₂ and CO—CH), 4.2 (m, 5H, N—CH₂—CO), 5.15 (s, 4H, Ar—CH₂), 5.45 (bs, 1H, Boc-NH), 7.3 (m, 10H, Ar—H) and 7.6 (2bs, 2H, CONH). M.S.—m/z 564.1 [M+H]. Analytical HPLC retention time—8.29 min (>97% pure, 20-65% B over 15 min).

### B. H-Glu(G-OBn)-G-OBn

The fully protected glutamic acid derivative (1.7 g, 3.2 mmol) B from above was dissolved in DCM/TFA (4:1, 20 mL) and stirred until the starting material disappeared on TLC (2 h). The reaction mixture was poured into ice cold saturated sodium bicarbonate solution (200 mL) and the organic layer was separated and the aqueous layer was extracted with 2×50 mL of DCM and combined with the organic layer. The DCM layer was washed with saturated sodium bicarbonate (2×100 mL), water (2×100 mL) and dried (sodium sulfate). The solution was filtered and evaporated under reduced pressure and the residue was dried under vacuum to yield a glass (Compound C, FIG. 29A) that was taken to the next step without further purification. Yield: 0.72 g (95%). M.S.—m/z 442.2 [M+H].

### C. (DOTA-tri-t-butyl)-Glu-(G-OBn)-G-OBn

The amine C from above (1.33 g, 3 mmol) in anhydrous DCM (10.0 mL) was added to an activated solution of DOTA-tri-t-butyl ester [2.27 g, 3.6 mmol was treated with HBTU, 1.36 g, 3.6 mmol and DIEA 1.04 g, 8 mmol and stirred for 30 min at RT in 25 mL of dry DCM] and stirred at RT for 20 h]. The reaction mixture was diluted with 200 mL of DCM and washed with saturated sodium carbonate (2×150 mL) and dried (sodium sulfate). The solution was filtered and solvent was removed under reduced pressure to yield a brown paste. The crude product was chromatographed over flash silica gel (500.0 g). Elution with 2% methanol in DCM furnished the product as a colorless gum (Compound D, FIG. 29A). Yield: 1.7 g (56.8%). ¹H NMR (CDCl₃): δ 1.3 and 1.4 (2s, 9H, three methyls each from the free base and the sodium adduct of DOTA), 2.0-3.5 (m, 20H, N—CH₂s and —CH—CH₂ and CO—CH₂), 3.75-4.5 (m, 13H, N—CH₂—CO), 5.2 (m, 4H, Ar—CH₂) and 7.25 (m, 10H, Ar—H). M. S. m/z—1018.3 [M+Na] and 996.5 [M+H] and 546.3 [M+Na⁺H]/2. HPLC—Retention Time: 11.24 min (>90%, 20-80% B over 30 min).

### D. (DOTA-tri-t-butyl)-Glu-(G-OH)-G-OH

The bis benzyl ester (0.2 g, 0.2 mmol) D from above was dissolved in methanol-water (20 mL, 9:1) and hydrogenated at 50 psi in the presence of 10% Pd/C catalyst (0.4 g, 50% by wt. water). After the starting material disappeared on HPLC and TLC (4 h), the solution was filtered off the catalyst and the solvent was removed under reduced pressure and the residue was dried under high vacuum for about 20 h (<0.1 mm) to yield the product as a colorless foam (Compound E, FIG. 29A). Yield: 0.12 g (73.5%). ¹H NMR (DMSO-d₆): δ 1.3 and 1.4 (2s, 9H corresponding to methyls of free base and the sodium adduct of DOTA), 1.8-4.7 (m, 33H, NCH₂s, COCH₂ and CH—CH₂ and NH—CH—CO), 8.1, 8.2 and 8.4 (3bs, NHCO). M. S.: m/z—816.3 [M+H] and 838.3 [M+Na]. HPLC Retention Time: 3.52 min (20-80% B over 30 min, >95% pure).

### E. H-8-amino-3,6-dioxaoctanoyl-8-amino-3,6-dioxaoctanoyl-Gln-Trp-Ala-Val-Gly-His-Leu-Met-NH

Fmoc-Q(Trt)-W(Boc)-A-V-G-H(Trt)-L-M-resin (0.5 g, 0.2 mmol) A was deblocked and coupled twice sequentially to 8-amino-3,6-dioxaoctanoic acid to yield the above deprotected peptide (Compound F, FIG. 29B) after preparative HPLC purification. Yield: 91.0 mg (37%).

HPLC Retention Time: 8.98 min (>95% purity, 10-40% B in over 10 min). M. S.: m/z—1230.6 [M+H], 615.9 [M+2H]/2.

### F. Solution Phase Coupling of the Bis-Acid E and the Amine F from Above

### FIG. 29B

The bis-acid (13.5 mg, 0.0166 mmol) E was dissolved in 100 μL of dry acetonitrile and treated with NHS (4.0 mg, 0.035 mmol) and DIC (5.05 mg, 0.04 mmol) and stirred for 24 h at RT. To the above activated acid, the free amine F (51.0 mg, 0.41 mmol)[generated from the TFA salt by treatment with saturated sodium bicarbonate and freeze drying the solution to yield the amine as a fluffy solid] was added followed by 100 μL of NMP and the stirring was continued for 40 h more at RT. The solution was diluted with anhydrous ether (10 mL) and the precipitate was collected by centrifugation and washed with 2×10 mL of anhydrous ether again. The crude solid was then purified by preparative HPLC to yield the product as a colorless fluffy solid L209 as in FIG. 29B with a yield of 7.5 mg (14.7%).

### Example XXIII

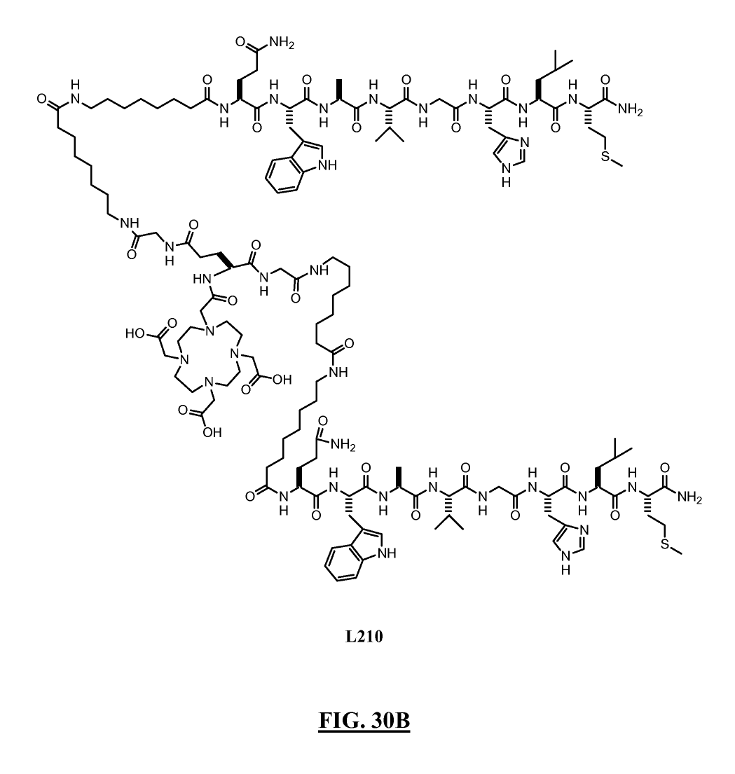

### FIGS. 30A-B

### Synthesis of L210

### A. H-8-aminooctanovl-8-aminooctanovl-Gln-Trp-Ala-Val-Gly-His-Leu-Met-NH₂

This was also prepared exactly the same way as in the case of Compound F (FIG. 29B), but using 1-aminooctanoic acid and the amine (Compound B, FIG. 30A) was purified by preparative HPLC. Yield: 95.0 mg (38.9%). HPLC Retention Time: 7.49 min (>95% purity; 10-40% B over 10.0 min). M. S.: m/z—1222.7 [M+H], 611.8 [M+2H]/2.

(DOTA-tri-t-butyl)-Glu-(G-OH)-G-OH (0.0163 g, 0.02 mmol) was converted to its bis-NHS ester as in the case of L209 in 100 μL of acetonitrile and treated with the free base, Compound B (60.0 mg, 0.05 mmol) in 100 μL of NMP and the reaction was continued for 40 h and then worked up and purified as above to prepare L210 (FIG. 30B) for a yield of 11.0 mg (18%).

### Example XXIV

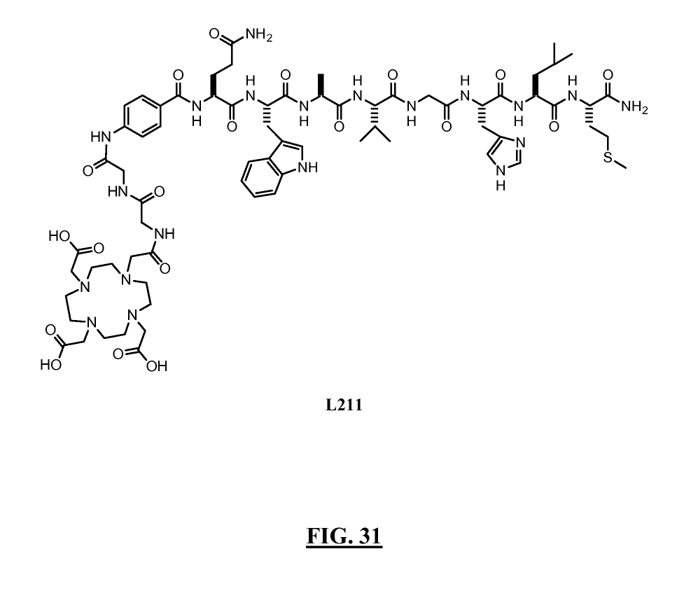

### FIG. 31

### Synthesis of L211

Prepared from 0.2 g Of the Fmoc-Q(Trt)-W(Boc)-A-V-G-H(Trt)-L-M-resin (0.08 mmol) using standard protocols. N-[(3β,5β,12α)-3-[[[4,7,10-Tris(carboxymethyl)-1,4,7,10-tetraazacyclododec-1-yl]acetyl]amino]-glycyl-glycyl-4-aminobenzoyl-L-glutaminyl-L-tryptophyl-L-alanyl-L-valyl-glycyl-L-histidyl-L-leucyl-L-methioninamide L211 was prepared in a yield of 4.7 mg (3.7%) (FIG. 31).

### Example XXV

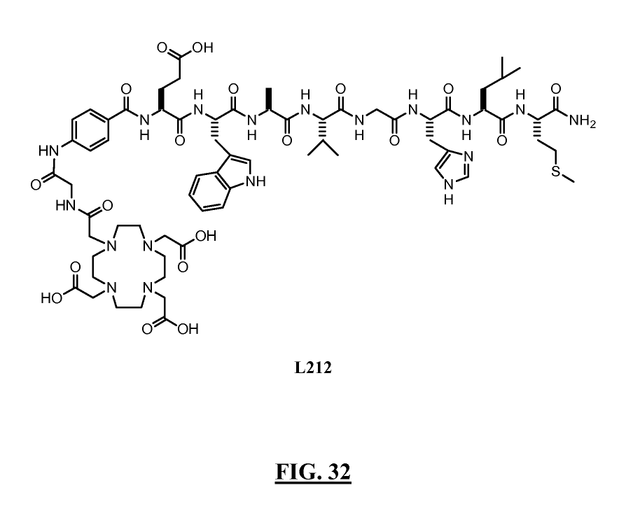

### FIG. 32

### Synthesis of L212

Prepared from Rink Amide Novagel resin (0.47 mmol/g, 0.2 g, 0.094 mmol) by building the sequence on the resin by standard protocols. N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutamyl-L-tryptophyl-L-alanyl-L-valyl-glycyl-L-histidyl-L-leucyl-L-methioninamide L212 was prepared for a yield of 25.0 mg (17.7%) (FIG. 32).

### Example XXVI

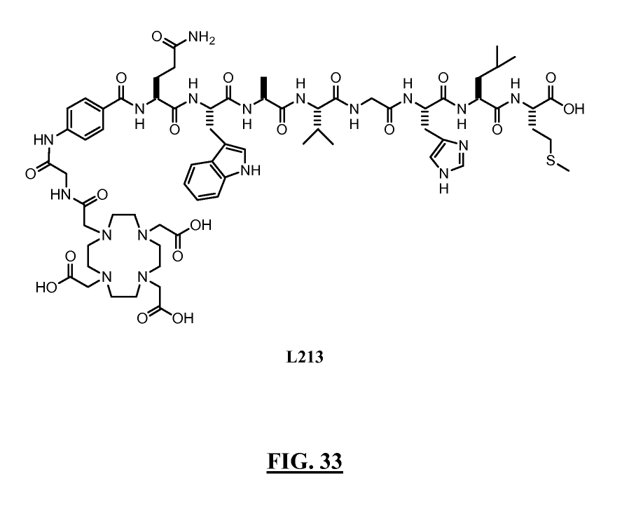

### FIG. 33

### Synthesis of L213

Prepared from Fmoc-Met-2-chlorotrityl chloride resin (NovaBioChem, 0.78 mmol/g, 0.26 g, 0.2 mmol) and the rest of the sequence were built using standard methodology. N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutaminyl-L-tryptophyl-L-alanyl-L-valyl-glycyl-L-histidyl-L-leucyl-L-methionine L213 was prepared for a yield of 49.05 mg (16.4%) (FIG. 33).

### Example XXVII

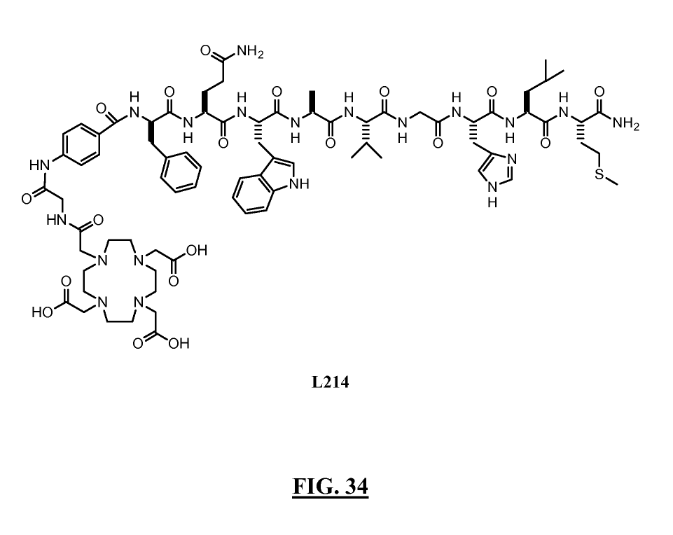

### FIG. 34

### Synthesis of L214

Fmoc-Q(Trt)-W(Boc)-A-V-G-H(Trt)-L-M-resin (0.2 g, 0.08 mmol) A was used to prepare N-[(3β,5β,12α)-3-[[[4,7,10-Tris(carboxymethyl)-1,4,7,10-tetraazacyclododec-1-yl]acetyl]amino]-glycyl-4-aminobenzoyl-D-phenylalanyl-L-glutaminyl-L-tryptophyl-L-alanyl-L-valyl-glycyl-L-histidyl-L-leucyl-L-methioninamide L214 using standard conditions. 8.5 mg of the product (6.4%) was obtained (FIG. 34).

### Example XXVIII

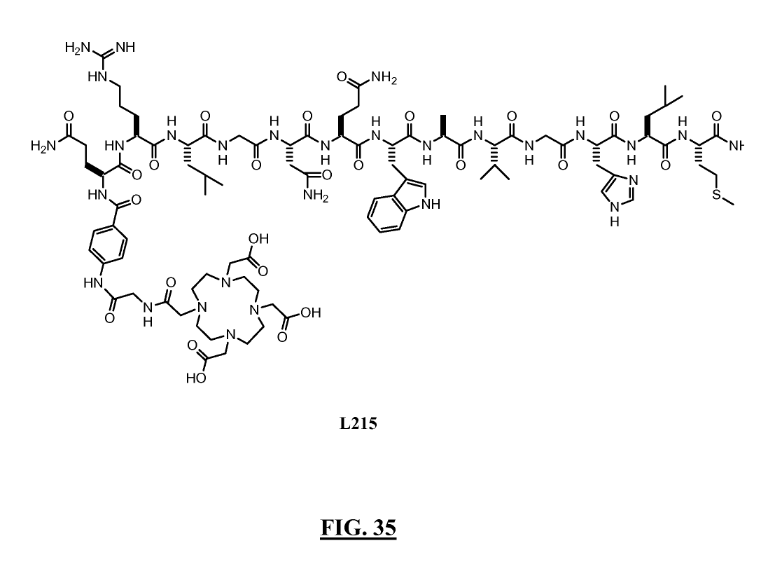

### FIG. 35

### Synthesis of L215

Fmoc-Q(Trt)-W(Boc)-A-V-G-H(Trt)-L-M-resin (0.2 g, 0.08 mmol) A was used to prepare N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutaminyl-L-arginyl-L-leucyl-glycyl-L-asparginyl-L-glutaminyl-L-tryptophyl-L-alanyl-L-valyl-glycyl-L-histidyl-L-leucyl-L-methioninamide L215. 9.2 mg (5.5%) was obtained (FIG. 35).

### Example XXIX

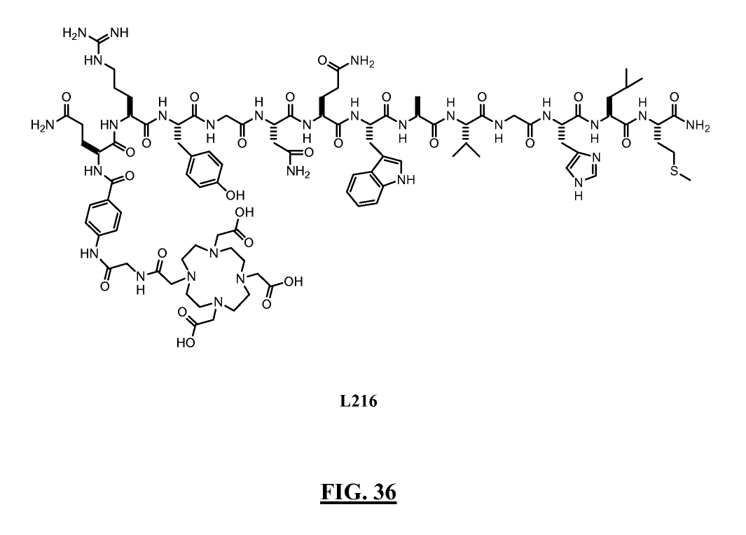

### FIG. 36

### Synthesis of L216

Fmoc-Q(Trt)-W(Boc)-A-V-G-H(Trt)-L-M-resin (0.2 g, 0.08 mmol) A was used to prepare N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutaminyl-arginyl-L-tyrosinyl-glycyl-L-asparginyl-L-glutaminyl-L-tryptophyl-L-alanyl-L-valyl-glycyl-L-histidyl-L-leucyl-L-methioninamide L216. 25.0 mg (14.7%) was obtained (FIG. 36).

### Example XXX

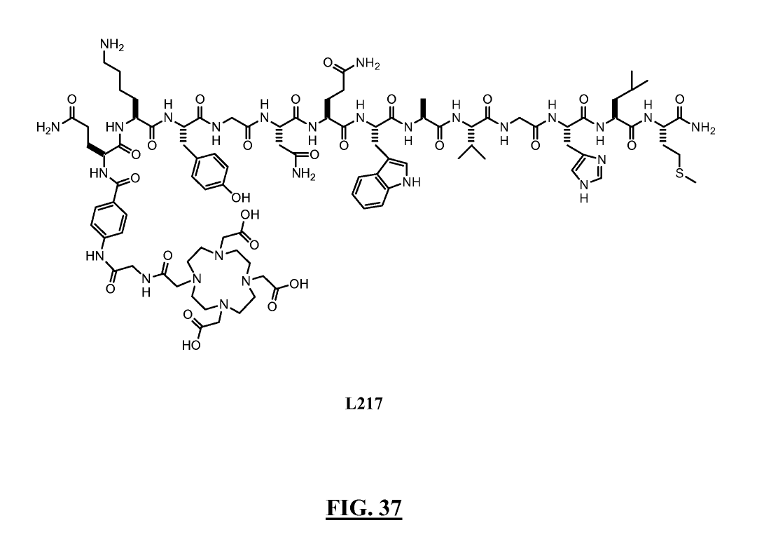

### FIG. 37

### Synthesis of L217

Fmoc-Q(Trt)-W(Boc)-A-V-G-H(Trt)-L-M-resin A (0.2 g, 0.08 mmol) was used to prepare N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutaminyl-L-lysyl-L-tyrosinyl-glycyl-L-glutaminyl-L-tryptophyl-L-alanyl-L-valyl-glycyl-L-histidyl-L-leucyl-L-methioninamide L217. 58.0 mg (34.7%) was obtained (FIG. 37).

### Example XXXI

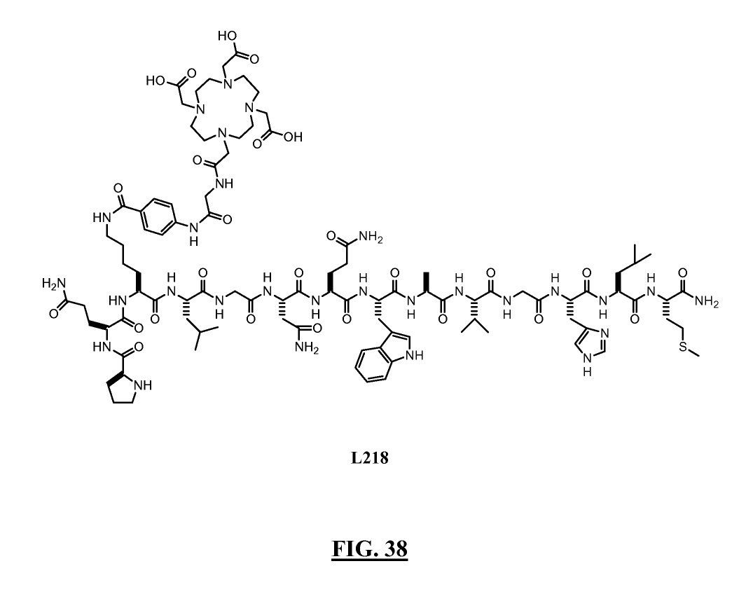

### FIG. 38

### Synthesis of L218

Fmoc-Q(Trt)-W(Boc)-A-V-G-H(Trt)-L-M-resin A (0.2 g, 0.08 mmol) was used. Fmoc-Lys(ivDde) was employed for the introduction of lysine. After the linear sequence was completed, the protecting group of the lysine was removed using 10% hydrazine in DMF (2×mL; 10 min each and then washed). The rest of the amino acids were then introduced using procedures described in the “general” section to complete the required peptide sequence. L218 in FIG. 38 as obtained in a yield of 40.0 mg (23.2%).

### Example XXXII

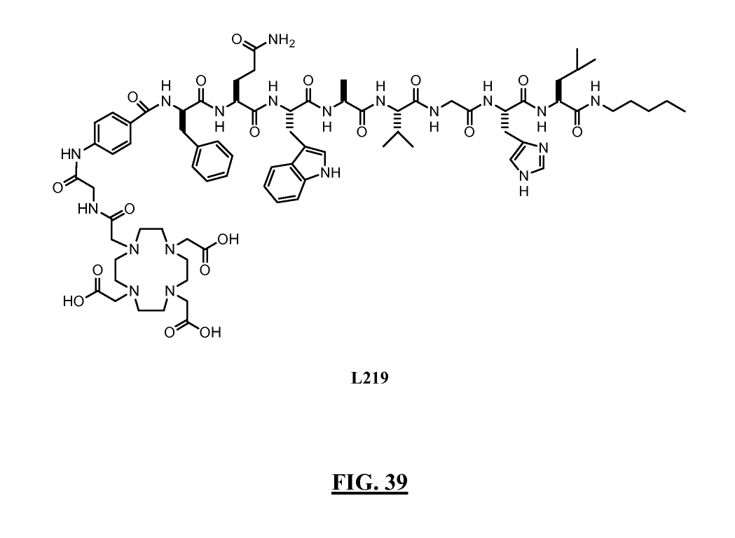

### FIG. 39

### Synthesis of L219

4-Sulfamylbutyryl AM Novagel resin was used (1.1 mmol/g; 0.5 g; 0.55 mmol). The first amino acid was loaded on to this resin at −20° C. for 20 h. The rest of the sequence was completed utilizing normal coupling procedures. After washing, the resin was alkylated with 20.0 eq. of iodoacetonitrile and 10.0 equivalents of DIEA for 20 h. The resin was then drained of the liquids and washed and then cleaved with 2.0 eq. of pentylamine in 5.0 mL of THF for 20 h. The resin was then washed with 2×5.0 mL of THF and all the filtrates were combined. THF was then evaporated under reduced pressure and the residue was then deblocked with 10.0 mL of Reagent B and the peptide N-[(3β,5β,12α)-3-[[[4,7,10-Tris(carboxymethyl)-1,4,7,10-tetraazacyclododec-1-yl]acetyl]amino]-glycyl-4-aminobenzoyl-D-phenylalanyl-L-glutaminyl-L-tryptophyl-L-alanyl-L-valyl-glycyl-L-histidyl-L-leucyl-aminopentyl, L219 was purified as previously described. 28.0 mg (2.8%) was obtained (FIG. 39).

### Example XXXIII

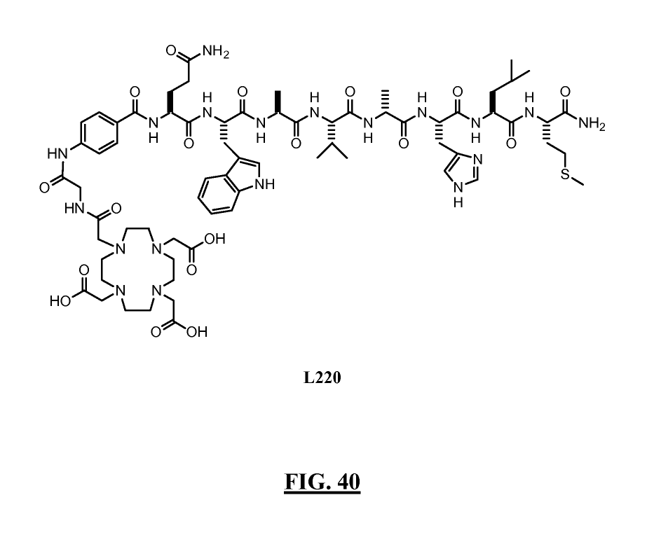

### FIG. 40

### Synthesis of L220

NovaSyn TGR (0.25 mmol/g; 0.15 g, 0.05 mmol) resin A was used to prepare N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutaminyl-L-tryptophyl-L-alanyl-L-valyl-D-alanyl-L-histidyl-L-leucyl-L-methioninamide, L220. 31.5 mg (41.4%) was obtained (FIG. 40).

### Example XXXIV

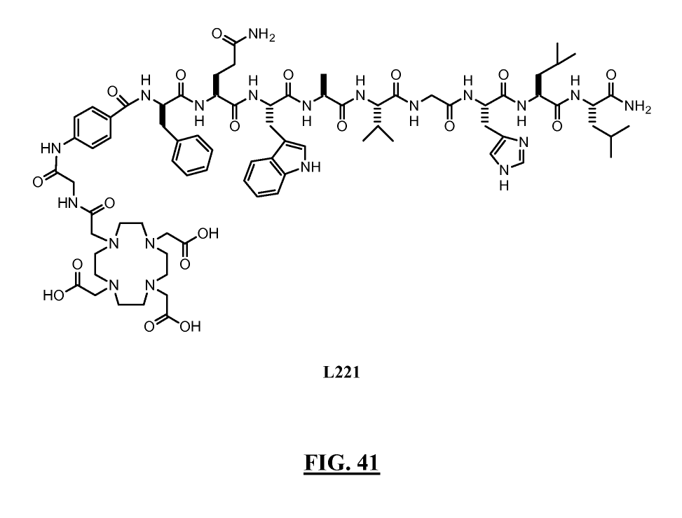

### FIG. 41

### Synthesis of L221

NovaSyn TGR (0.25 mmol/g; 0.15 g, 0.05 mmol) resin A was used to prepare N-[(3β,5β,12α)-3-[[[4,7,10-Tris(carboxymethyl)-1,4,7,10-tetraazacyclododec-1-yl]acetyl]amino]-glycyl-4-aminobenzoyl-D-phenylalanyl-L-glutaminyl-L-tryptophyl-L-alanyl-L-valyl-glycyl-L-histidyl-L-leucinamide, L221. 28.0 mg (34.3%) was obtained (FIG. 41).

### Example XXXV

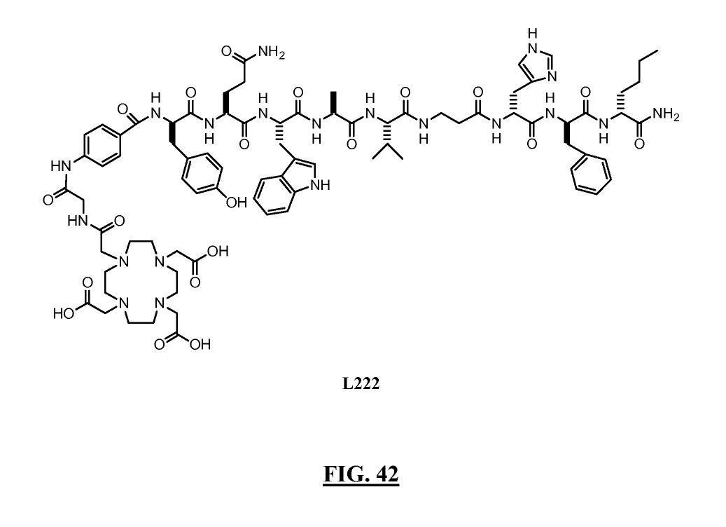

### FIG. 42

### Synthesis of L222

NovaSyn TGR (0.25 mmol/g; 0.15 g, 0.05 mmol) resin A was used to prepare N-[(3β,5β,12α)-3-[[[4,7,10-Tris(carboxymethyl)-1,4,7,10-tetraazacyclododec-1-yl]acetyl]amino]-glycyl-4-aminobenzoyl-D-tyrosinyl-L-glutaminyl-L-tryptophyl-L-alanyl-L-valyl-betaalanyl-L-histidyl-L-phenylalanyl-L-norleucinamide, L222. 34.0 mg (40.0%) was obtained (FIG. 42).

### Example XXXVI

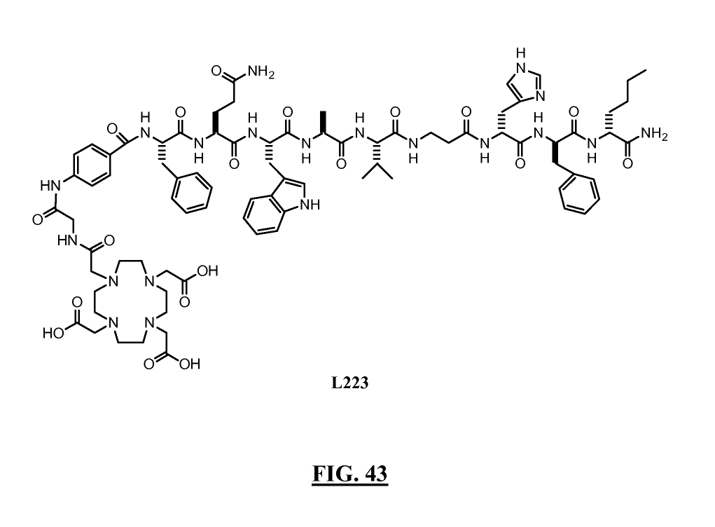

### FIG. 43

### Synthesis of L223

NovaSyn TGR (0.25 mmol/g; 0.15 g, 0.05 mmol) resin A was used to prepare N-[(3β,5β,12α)-3-[[[4,7,10-Tris(carboxymethyl)-1,4,7,10-tetraazacyclododec-1-yl]acetyl]amino]-glycyl-4-aminobenzoyl-L-phenylalanyl-L-glutaminyl-L-tryptophyl-L-alanyl-L-valyl-betaalanyl-L-histidyl-L-phenylalanyl-L-norleucinamide, L223. 31.2 mg (37.1%) was obtained (FIG. 43).

### Example XXXVII

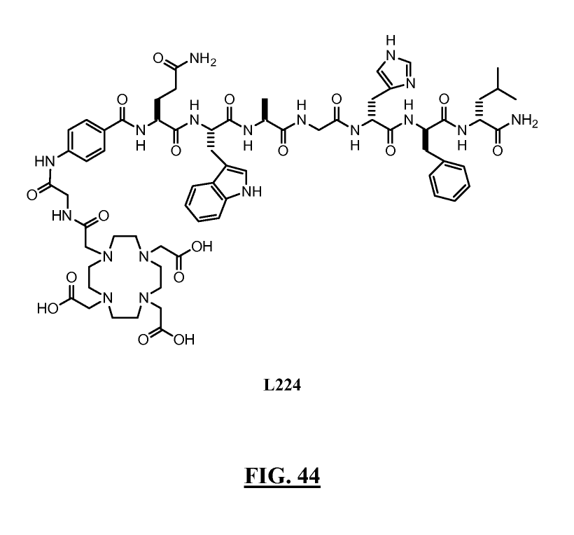

### FIG. 44

### Synthesis of L224

NovaSyn TGR (0.25 mmol/g; 0.15 g, 0.05 mmol) resin A was used to prepare N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutaminyl-L-tryptophyl-L-alanyl-glycyl-L-histidyl-L-phenylalanyl-L-leucinamide, L224. 30.0 mg (42.2%) was obtained (FIG. 44).

### Example XXXVIII

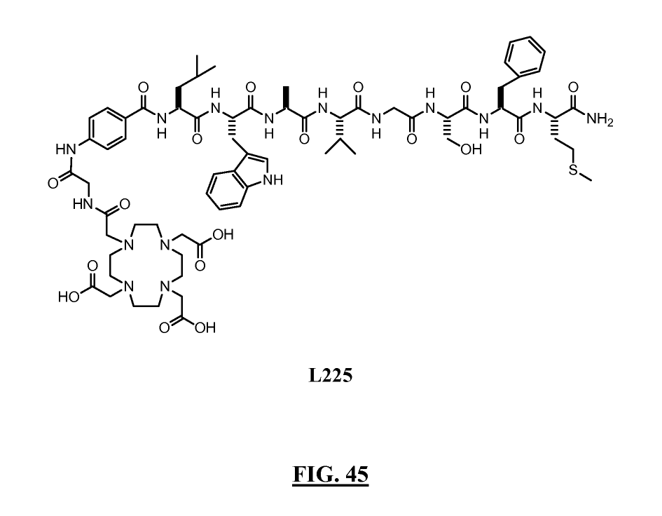

### FIG. 45

### Synthesis of L225

NovaSyn TGR (0.25 mmol/g; 0.15 g, 0.05 mmol) resin A was used to prepare N-[(3β,5β,12α)-3-[[[4,7,10-Tris(carboxymethyl)-1,4,7,10-tetraazacyclododec-1-yl]acetyl]amino]-glycyl-4-aminobenzoyl-L-leucyl-L-tryptophyl-L-alanyl-L-valinyl-glycyl-L-serinyl-L-phenylalanyl-L-methioninamide, L225. 15.0 mg (20.4%) was obtained (FIG. 45).

### Example XXXIX

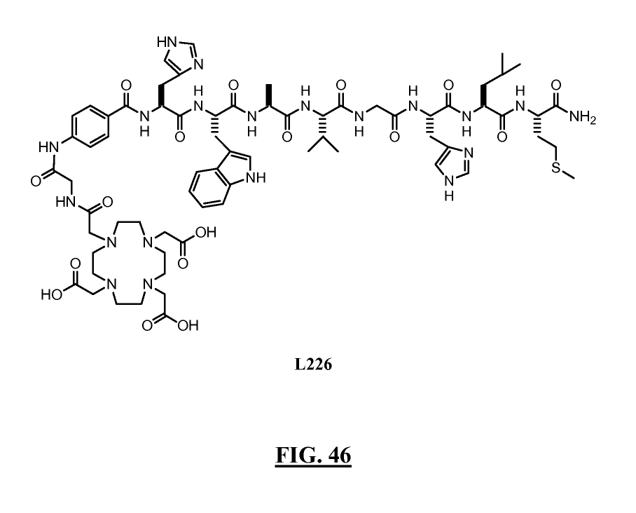

### FIG. 46

### Synthesis of L226

NovaSyn TGR (0.25 mmol/g; 0.15 g, 0.05 mmol) resin A was used to prepare N-[(3β,5β,12α)-3-[[[4,7,10-Tris(carboxymethyl)-1,4,7,10-tetraazacyclododec-1-yl]acetyl]amino]-glycyl-4-aminobenzoyl-L-histidyl-L-tryptophyl-L-alanyl-L-valyl-glycyl-L-histidyl-L-leucyl-L-methioninamide, L226. 40.0 mg (52.9%) was obtained (FIG. 46).

### Example XL

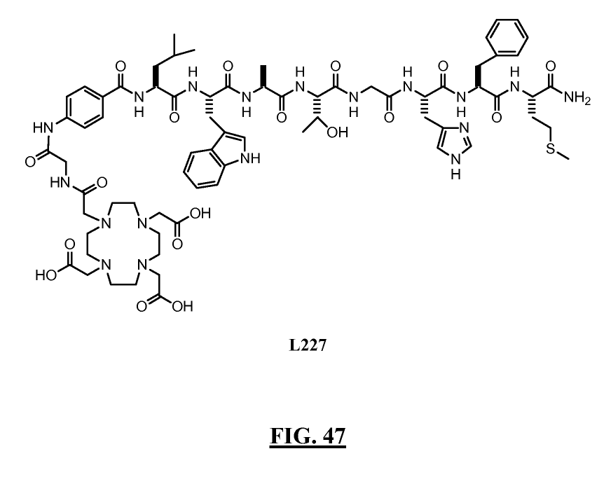

### FIG. 47

### Synthesis of L227

NovaSyn TGR (0.25 mmol/g; 0.15 g, 0.05 mmol) resin A was used to prepare N-[(3β,5β,12α)-3-[[[4,7,10-Tris(carboxymethyl)-1,4,7,10-tetraazacyclododec-1-yl]acetyl]amino]-glycyl-4-aminobenzoyl-L-leucyl-L-tryptophyl-L-alanyl-L-threonyl]-glycyl-L-histidyl-L-phenylalanyl-L-methioninamide L227. 28.0 mg (36.7%) was obtained (FIG. 47).

### Example XLI

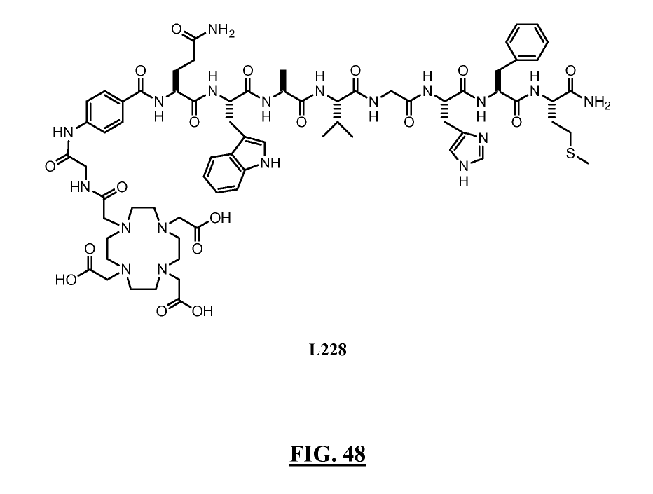

### FIG. 48

### Synthesis of L228

NovaSyn TGR (0.25 mmol/g; 0.15 g, 0.05 mmol) resin A was used to prepare N-[(3β,5β,12α)-3-[[[4,7,10-Tris(carboxymethyl)-1,4,7,10-tetraazacyclododec-1-yl]acetyl]amino]-glycyl-4-aminobenzoyl-L-glutaminyl-L-tryptophyl-L-alanyl-L-valyl-glycyl-L-histidyl-L-phenylalanyl-L-methioninamide, L228. 26.0 mg (33.8%) was obtained (FIG. 48).

### Example XLII

### Synthesis of Additional GRP Compounds

### A. General Procedure for the preparation of 4,4′-Aminomethylbiphenylcarboxylic acid (B2) and 3,3′-aminomethylbiphenylcarboxylic acid (B3)

### 1. Methyl-hydroxymethylbiphenylcarboxylates

Commercially available (Aldrich Chemical Co.) 4-hydroxymethylphenylboric acid or 3-hydroxymethylphenylboric acid (1.0 g, 6.58 mmol) was stirred with isopropanol (10 mL) and 2M sodium carbonate (16 mL) until the solution became homogeneous. The solution was degassed by passing nitrogen through the solution and then treated with solid methyl-3-bromobenzoate, or methyl-4-bromobenzoate (1.35 g, 6.3 mmol) followed by the Pd (O) catalyst {[(C₆Hs)₃P]₄Pd; 0.023 g, 0.003 mmol}. The reaction mixture was kept at reflux under nitrogen until the starting bromobenzoate was consumed as determined by TLC analysis (2-3 h). The reaction mixture was then diluted with 250 mL of water and extracted with ethyl acetate (3×50 mL). The organic layers were combined and washed with saturated sodium bicarbonate solution (2×50 mL) and dried (Na₂SO₄). The solvent was removed under reduced pressure and the residue was chromatographed over flash silica gel (100 g). Elution with 40% ethyl acetate in hexanes yielded the product either as a solid or oil.

Yield:

B2—0.45 g (31%); m.p.—170-171° C.

B3—0.69 g (62%); oil.

¹H NMR (CDCl₃) δ B2—3.94 (s, 3H, —COOCH₃), 4.73 (s, 2H, —CH₂-Ph), 7.475 (d, 2H, J=5 Hz), 7.6 (d, 2H, J=10 Hz), 7.65 (d, 2H, J=5 Hz) and 8.09 (d, 2H, J=10 Hz).

M.S.—m/e—243.0 [M+H]

B3—3.94 (s, 3H, —COOCH₃), 4.76 (s, 2H, —CH₂-Ph), 7.50 (m, 4H), 7.62 (s, 1H), 7.77 (s, 1H), 8.00 (s, 1H) and 8.27 (s, 1H).

M.S.—m/e—243.2 [M+H]

### 2. Azidomethylbiphenyl Carboxylates

The above biphenyl alcohols (2.0 mmol) in dry dichloromethane (10 mL) were cooled in ice and treated with diphenylphosphoryl azide (2.2 mol) and DBU (2.0 mmol) and stirred under nitrogen for 24 h. The reaction mixture was diluted with water and extracted with ethyl acetate (2×25 mL).The organic layers were combined and washed successfully with 0.5 M citric acid solution (2×25 mL), water (2×25 mL) and dried (Na₂SO₄). The solution was filtered and evaporated under reduced pressure to yield the crude product. The 4,4′-isomer was crystallized from hexane/ether and the 3,3′-isomer was triturated with isopropyl ether to remove all the impurities; the product was homogeneous as determined on TLC analysis and further purification was not required.

Yield:

Methyl-4-azidomethyl-4-biphenylcaroxylate—0.245 g (46%); m.p.—106-108° C.

Methyl-4-azidomethyl-4-biphenylcaroxylate—0.36 g (59%, oil)

¹H NMR (CDCl₃) δ—4,4′-isomer—3.95 (s, 3H, —COOCH₃), 4.41 (s, 2H, —CH₂N₃), 7.42 (d, 2H, J=5 Hz), 7.66 (m, 4H) and 8.11 (d, 2H, J=5 Hz)

3,3′—Isomer—3.94 (s, 3H, —COOCH₃), 4.41 (s, 2H, —CH₂N₃), 7.26-7.6 (m, 5H), 7.76 (d, 1H, J=10 Hz), 8.02 (d, 1H, J=5 Hz) and 8.27 (s, 1H).

### 3. Hydrolysis of the Methyl Esters of Biphenylcarboxylates

About 4 mmol of the methyl esters were treated with 20 mL of 2M lithium hydroxide solution and stirred until the solution was homogeneous (20-24 h). The aqueous layer was extracted with 2×50 mL of ether and the organic layer was discarded. The aqueous layer was then acidified with 0.5 M citric acid and the precipitated solid was filtered and dried. No other purification was necessary and the acids were taken to the next step.

Yield:

4,4′-isomer—0.87 g of methyl ester yielded 0.754 g of the acid (86.6%); m.p.—205-210° C.

3,3′-isomer—0.48 g of the methyl ester furnished 0.34 g of the acid (63.6%); m.p.—102-105° C.

¹H NMR (DMSO-d₆) δ: 4,4′-isomer—4.52 (s, 2H, —CH₂N₃), 7.50 (d, 2H, J=5 Hz), 7.9 (m, 4H), and 8.03 (d, 2H, J=10 Hz)

3,3′-isomer—4.54 (s, 2H, —CH₂N₃), 7.4 (d, 1H, J=10 Hz), 7.5-7.7 (m, 4H), 7.92 (ABq, 2H) and 8.19 (s, 1H).

### 4. Reduction of the Azides to the Amine

This was carried out on the solid phase and the amine was never isolated. The azidocarboxylic acid was loaded on the resin using the standard peptide coupling protocols. After washing, the resin containing the azide was shaken with 20 equivalents of triphenylphosphine in THF/water (95:5) for 24 h. The solution was drained under a positive pressure of nitrogen and then washed with the standard washing procedure. The resulting amine was employed in the next coupling.

### 5. (3β,5β,7α,12α)-3-[{(9H-Flouren-9ylmethoxy)amino]acetyl}amino-7,12-dihydroxycholan-24-oic acid

Tributylamine (3.2 mL); 13.5 mmol) was added dropwise to a solution of Fmoc-glycine (4.0 g, 13.5 mmol) in THF (80 mL) stirred at 0° C. Isobutylchloroformate (1.7 mL; 13.5 mmol) was subsequently added and, after 10 min, a suspension of tributylamine (2.6 mL; 11.2 mmol) and (3β,5β,7α,12α)-3-amino-7,12-dihydroxycholan-24-oic acid (4.5 g; 11.2 mmol) in DMF (80 mL) was added dropwise, over 1 h, into the cooled solution. The mixture was allowed to warm up to ambient temperature and after 6 h, the solution was concentrated to 120 mL, then water (180 mL) and 1N HCl (30 mL) were added (final pH 1.5). The precipitated solid was filtered, washed with water (2×100 mL), vacuum dried and purified by flash chromatography. Elution with chloroform/methanol (8:2) yielded the product as a colorless solid.

Yield: 1.9 g (25%). TLC: R_{f} 0.30 (CHCl₃/MeOH/NH₄OH—6:3:1).

### In Vitro and In Vivo Testing of Compounds

### Example XLIII

### In Vitro Binding Assay for GRP Receptors in PC-3 Cell Lines

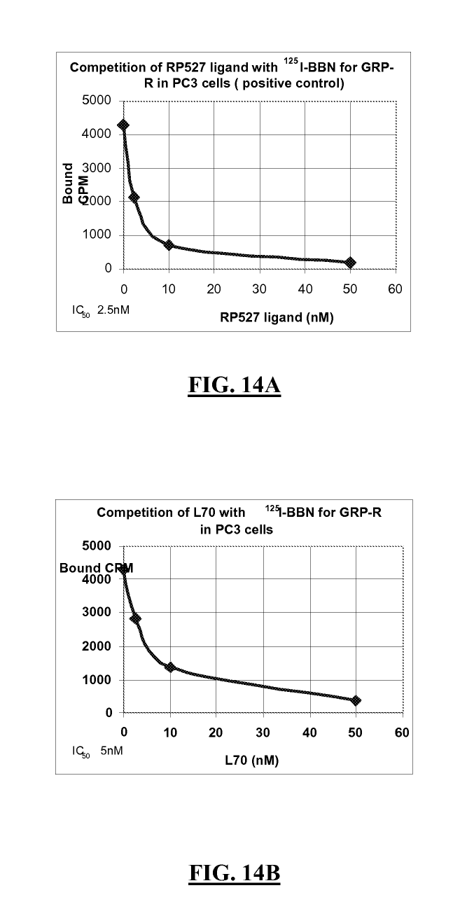

### FIGS. 14 A-B

To identify potential lead compounds, an in vitro assay that identifies compounds with high affinity for GRP-R was used. Since the PC3 cell line, derived from human prostate cancer, is known to exhibit high expression of GRP-R on the cell surface, a radio ligand binding assay in a 96-well plate format was developed and validated to measure the binding of ¹²⁵I-BBN to GRP-R positive PC3 cells and the ability of the compounds of the invention to inhibit this binding. This assay was used to measure the IC₅₀ for RP527 ligand, DO3A-monoamide-Aoc-QWAVGHLM-NH₂ (controls) and compounds of the invention which inhibit the binding of ¹²⁵I-BBN to GRP-R. (RP527=N,N-dimethylglycine-Ser-Cys(Acm)-Gly-5-aminopentanoic acid-BBN (7-14), which is SEQ ID NO: 1), which has MS=1442.6 and IC50-0.84). Van de Wiele C, Dumont F et al., Technetium-99m RP527, a GRP analogue for visualization of GRP receptor-expressing malignancies: a feasibility study. Eur. J. Nucl. Med., (2000) 27; 1694-1699.; DO3A-monoamide-Aoc-QWAVGHLM-NH₂ is also referred to as DO3A-monoamide-8-amino-octanoic acid-BBN (7-14), which is SEQ ID NO: 1, and has MS=1467.0. DO3A monoamide-aminooctanyl-BBN[7-14]

The Radioligand Binding Plate Assay was validated for BBN and BBN analogues (including commercially available BBN and L) and also using ^{99m}Tc RP527 as the radioligand.

A. Materials and Methods:

1. Cell Culture:

PC3 (human prostate cancer cell line) were obtained from the American Type Culture Collection and cultured in RPMI 1640 (ATCC) in tissue culture flasks (Corning). This growth medium was supplemented with 10% heat inactivated FBS (Hyclone, SH30070.03), 10 mM HEPES (GibcoBRL, 15630-080), and antibiotic/antimycotic (GibcoBRL, 15240-062) for a final concentration of penicillin-streptomycin (100 units/mL), and fungizone (0.25 μg/mL). All cultures were maintained in a humidified atmosphere containing 5% CO₂/95% air at 37° C., and passaged routinely using 0.05% trypsin/EDTA (GibcoBRL 25300-054) where indicated. Cells for experiments were plated at a concentration of 2.0×10⁴/well either in 96-well white/clear bottom microtiter plates (Falcon Optilux-I) or 96 well black/clear collagen I cellware plates (Beckton Dickinson Biocoat). Plates were used for binding studies on day 1 or 2 post-plating.

2. Binding Buffer:

RPMI 1640 (ATCC) supplemented with 20 mM HEPES, 0.1% BSA (w/v), 0.5 mM PMSF (AEBSF), Bacitracin (50 mg/500 ml), pH 7.4. ¹²⁵I-BBN (carrier free, 2200 Ci/mmole) was obtained from Perkin-Elmer.

B. Competition Assay with ¹²⁵I-BBN for GRP-R in PC3 Cells:

A 96-well plate assay was used to determine the IC₅₀ of various compounds of the invention to inhibit binding of ¹²⁵I-BBN to human GRP-R. The following general procedure was followed:

All compounds tested were dissolved in binding buffer and appropriate dilutions were also done in binding buffer. PC3 cells (human prostate cancer cell line) for assay were plated at a concentration of 2.0×10⁴/well either in 96-well white/clear bottomed microtiter plates (Falcon Optilux-I) or 96 well black/clear collagen I cellware plates (Beckton Dickinson Biocoat). Plates were used for binding studies on day 1 or 2 post-plating. The plates were checked for confluency (>90% confluent) prior to assay. For the assay, RP527 or DO3A-monoamide-Aoc-QWAVGHLM-NH₂ ligand, (controls), or compounds of the invention at concentrations ranging from 1.25×10⁻⁹ M to 5×10⁻⁹ M, was co-incubated with ¹²⁵I-BBN (25,000 cpm/well). These studies were conducted with an assay volume of 75 μl per well. Triplicate wells were used for each data point. After the addition of the appropriate solutions, plates were incubated for 1 h at 4° C. to prevent internalization of the ligand-receptor complex. Incubation was ended by the addition of 200 μl of ice-cold incubation buffer. Plates were washed 5 times and blotted dry. Radioactivity was detected using either the LKB CompuGamma counter or a microplate scintillation counter.

Competition binding curves for RP527 (control) and L70, a compound of the invention can be found in FIGS. 14A-B. These data show that the IC₅₀ of the RP527 control is 2.5 nM and that of L70, a compound of this invention is 5 nM. The IC₅₀ of the DO3A-monoamide-Aoc-QWAVGHLM-NH₂ control was 5 nM. IC₅₀ values for those compounds of the invention tested can be found in Tables 1-3, supra, and show that they are comparable to that of the controls and thus would be expected to have sufficient affinity for the receptor to allow uptake by receptor bearing cells in vivo.

C. Internalization and Efflux Assay:

These studies were conducted in a 96-well plate. After washing to remove serum proteins, PC3 cells were incubated with ¹²⁵I-BBN, ¹⁷⁷Lu-DO3A-monoamide-Aoc-QWAVGHLM-NH₂ or radiolabeled compounds of this invention for 40 min, at 37° C. Incubations were stopped by the addition of 200 μl of ice-cold binding buffer. Plates were washed twice with binding buffer. To remove surface-bound radioligand, the cells were incubated with 0.2M acetic acid (in saline), pH 2.8 for 2 min. Plates were centrifuged and the acid wash media were collected to determine the amount of radioactivity which was not internalized. The cells were collected to determine the amount of internalized ¹²⁵I-BBN, and all samples were analyzed in the gamma counter. Data for the internalization assay was normalized by comparing counts obtained at the various time points with the counts obtained at the final time point (T=40 min).

For the efflux studies, after loading the PC3 cells with ¹²⁵I-BBN or radiolabeled compounds of the invention for 40 min at 37° C., the unbound material was filtered, and the % of internalization was determined as above. The cells were then resuspended in binding buffer at 37° C. for up to 3 h. At 0.5, 1, 2, or 3 h, the amount remaining internalized relative to the initial loading level was determined as above and used to calculate the percent efflux recorded in Table 9.

TABLE 9
Internalisation and efflux of ¹²⁵I-BBN and the Lu-177 complexes of
DO3A-monoamide-Aoc-QWAVGHLM-NH₂ (control) and
compounds of this invention

| | | DO3A-monoamide-Aoc- | | | |
|---|---|---|---|---|---|
| | | QWAVGHLM- | | | |
| | I-BBN | NH₂ (control) | L63 | L64 | L70 |
| Internalisation | 59 | 89 | 64 | 69 | 70 |
| (40 minutes) | | | | | |
| Efflux (2 h) | 35 | 28 | 0 | 20 | 12 |

These data show that the compounds of this invention are internalized and retained by the PC3 cells to a similar extent to the controls.

### Example XLIV

### Preparation of Tc-labeled GRP Compounds

Peptide solutions of compounds of the invention identified in Table 10 were prepared at a concentration of 1 mg/mL in 0.1% aqueous TFA. A stannous chloride solution was prepared by dissolving SnCl_{2*2}H₂O (20 mg/mL) in 1 N HCl. Stannous gluconate solutions containing 20 μg of SnCl₂.2H₂O/100 μL were prepared by adding an aliquot of the SnCl₂ solution (10 μL) to a sodium gluconate solution prepared by dissolving 13 mg of sodium gluconate in water. A hydroxypropyl gamma cyclodextrin [HP-γ-CD]solution was prepared by dissolving 50 mg of HP-γ-CD in 1 mL of water.

The ^{99m}Tc labeled compounds identified below were prepared by mixing 20 μL of solution of the unlabeled compounds (20 μg), 50 μL of HP-γ-CD solution, 100 μL of Sn-gluconate solution and 20 to 50 μL of ^{99m}Tc pertechnetate (5 to 8 mCi, Syncor). The final volume was around 200 μL and final pH was 4.5-5. The reaction mixture was heated at 100° C. for 15 to 20 min. and then analyzed by reversed phase HPLC to determine radiochemical purity (RCP). The desired product peaks were isolated by HPLC, collected into a stabilizing buffer containing 5 mg/mL ascorbic acid, 16 mg/mL HP-γ-CD and 50 mM phosphate buffer, pH 4.5, and concentrated using a speed vacuum to remove acetonitrile. The HPLC system used for analysis and purification was as follows: C18 Vydac column, 4.6×250 mm, aqueous phase: 0.1% TFA in water, organic phase: 0.085% TFA in acetonitrile. Flow rate: 1 mL/min. Isocratic elution at 20%-25% acetonitrile/0.085% TFA was used, depending on the nature of individual peptide.

Labeling results are summarized in Table 10.

TABLE 10

| | | HPLC | Ini- | RCP⁴ (%) |
|---|---|---|---|---|
| | | retention | tial | immediately |
| Com- | | time | RCP³ | following |
| pound¹ | Sequence² | (min) | (%) | purification |
| L2 | -RJQWAVGHLM- | 5.47 | 89.9 | 95.6 |
| | NH₂ | | | |
| L4 | -SJQWAVGHLM- | 5.92 | 65 | 97 |
| | NH₂ | | | |
| L8 | -JKQWAVGHLM- | 6.72 | 86 | 94 |
| | NH₂ | | | |
| L1 | -KJQWAVGHLM- | 5.43 | 88.2 | 92.6 |
| | NH₂ | | | |
| L9 | -JRQWAVGHLM- | 7.28 | 91.7 | 96.2 |
| | NH₂ | | | |
| L7 | -aJQWAVGHLM- | 8.47 | 88.6 | 95.9 |
| | NH₂ | | | |

n.d. = not detected
¹All compounds were conjugated with an N,N′-dimethylglycyl-Ser-Cys-Gly metal chelator. The Acm protected form of the ligand was used. Hence, the ligand used to prepare the 99mTc complex of L2 was N,N′-dimethylglycyl-Ser-Cys (Acm)-Gly-RJQWAVGHLM-NH₂. The Acm group was removed during chelation to Tc.
²In the Sequence,“J” refers to 8-amino-3,6-dioxaoctanoic acid and “a” refers to D-alanine
³Initial RCP measurement taken immediately after heating and prior to HPLC purification.
⁴RCP determined following HPLC isolation and acetonitrile removal via speed vacuum.

### Example XLV

### Preparation of ¹⁷⁷Lu-L64 for Cell Binding and Biodistribution Studies

This compound was synthesized by incubating 10 μg L64 ligand (10 μL of a 1 mg/mL solution in water), 100 μL ammonium acetate buffer (0.2M, pH 5.2) and ˜1-2 mCi of ¹⁷LuCl₃ in 0.05N HCl (MURR) at 90° C. for 15 min. Free ¹⁷Lu was scavenged by adding 20 μL of a 1% Na₂EDTA*2H₂O (Aldrich) solution in water. The resulting radiochemical purity (RCP) was ˜95%. The radiolabeled product was separated from unlabeled ligand and other impurities by HPLC, using a YMC Basic C8 column [4.6×150 mm], a column temperature of 30° C. and a flow rate of 1 mL/min, with a gradient of 68% A/32% B to 66% A/34% B over min., where A is citrate buffer (0.02M, pH 3.0), and B is 80% CH₃CN/20% CH₃OH. The isolated compound had an RCP of −100% and an HPLC retention time of 23.4 minutes.

Samples for biodistribution and cell binding studies were prepared by collecting the desired HPLC peak into 1000 μL of citrate buffer (0.05 M, pH 5.3, containing 1% ascorbic acid, and 0.1% HSA). The organic eluent in the collected eluate was removed by centrifugal concentration for 30 min. For cell binding studies, the purified sample was diluted with cell-binding media to a concentration of 1.5 μCi/mL within 30 minutes of the in vitro study. For biodistribution studies, the sample was diluted with citrate buffer (0.05 M, pH 5.3, containing 1% sodium ascorbic acid and 0.1% HSA) to a final concentration of 50 μCi/mL within 30 minutes of the in vivo study.

### Example XLVI

### Preparation of ¹⁷⁷Lu-L64 for Radiotherapy Studies

This compound was synthesized by incubating 70 μg L64 ligand (70 μL of a 1 mg/mL solution in water), 200 μL ammonium acetate buffer (0.2M, pH 5.2) and ˜30-40 mCi of ¹⁷⁷LuCl₃ in 0.05N HCl (MURR) at 85° C. for 10 min. After cooling to room temperature, free ¹⁷Lu was scavenged by adding 20 μL of a 2% Na₂EDTA*2H₂O (Aldrich) solution in water. The resulting radiochemical purity (RCP) was ˜95%. The radiolabeled product was separated from unlabeled ligand and other impurities by HPLC, using a 300VHP Anion Exchange column (7.5×50 mm) (Vydac) that was sequentially eluted at a flow rate of 1 mL/min with water, 50% acetonitrile/water and then 1 g/L aqueous ammonium acetate solution. The desired compound was eluted from the column with 50% CH₃CN and mixed with—1 mL of citrate buffer (0.05 M, pH 5.3) containing 5% ascorbic acid, 0.2% HSA, and 0.9% (v:v) benzyl alcohol. The organic part of the isolated fraction was removed by spin vacuum for 40 min, and the concentrated solution (˜20-25 mCi) was adjusted within 30 minutes of the in vivo study to a concentration of 7.5 mCi/mL using citrate buffer (0.05 M, pH 5.3) containing 5% ascorbic acid, 0.2% HSA, and 0.9% (v:v) benzyl alcohol. The resulting compound had an RCP of >95%.

### Example XLVII

### Preparation of ¹¹¹In-L64

This compound was synthesized by incubating 10 μg L64 ligand (5 μL of a 2 mg/mL solution in 0.01 N HCl), 60 μL ethanol, 1.12 mCi of ¹¹¹InCl₃ in 0.05N HCl (80 μL) and 155 μL sodium acetate buffer (0.5M, pH 4.5) at 85° C. for 30 min. Free ¹¹¹In was scavenged by adding 20 μL of a 1% Na₂EDTA.2H₂O (Aldrich) solution in water. The resulting radiochemical purity (RCP) was 87%. The radiolabeled product was separated from unlabeled ligand and other impurities by HPLC, using a Vydac C18 column, [4,6×250 mm], a column temperature of 50° C. and a flow rate of 1.5 mL/min. with a gradient of 75% A/25% B to 65% A/35% B over 20 min where A is 0.1% TFA in water, B is 0.085% TFA in acetonitrile. With this system, the retention time for ¹¹¹In-L64 is 15.7 min. The isolated compound had an RCP of 96.7%.

### Example XLVIII

### Preparation of ¹⁷⁷Lu-DO3A-monoamide-Aoc-OWAVGHLM-NH₂ (Control)

A stock solution of peptide was prepared by dissolving DO3A-monoamide-Aoc-QWAVGHLM-NH₂ ligand (prepared as described in US Application Publication No. 2002/0054855 and WO 02/87637, both incorporated by reference) in 0.01 N HCl to a concentration of 1 mg/mL. 17Lu-DO3A-monoamide-Aoc-QWAVGHLM-NH₂ was prepared by mixing the following reagents in the order shown.

| | |
|---|---|
| 0.2M NH₄OAc, pH 6.8 | 100 μL |
| Peptide stock, 1 mg/mL, in 0.01N HCl | 5 μL |
| ¹⁷⁷LuCl₃ (MURR) in 0.05M HCl | 1.2 μL (1.4 mCi) |

The reaction mixture was incubated at 85° C. for 10 min. After cooling down to room temperature in a water bath, 20 μL of a 1% EDTA solution and 20 μl of EtOH were added. The compound was analyzed by HPLC using a C18 column (VYDAC Cat #218TP54) that was eluted at flow rate of 1 mL/min with a gradient of 21 to 25% B over 20 min, where A is 0.1% TFA/H₂O and B is 0.1% TFA/CH₃CN). ¹⁷⁷Lu-DO3A-monoamide-Aoc-QWAVGHLM-NH₂ was formed in 97.1% yield (RCP) and had a retention time of −16.1 min on this system.

### Example XLIX

### Preparation of ¹⁷⁷Lu-L63

This compound was prepared as described for ¹⁷⁷Lu-DO3A-monoamide-Aoc-QWAVGHLM-NH₂, wherein QWAVGHLM-NH₂ is the BBN(7-14) sequence (SEQ ID NO: 1). The compound was analyzed by HPLC using a C18 column (VYDAC Cat #218TP54) that was eluted at flow rate of 1 mL/min with a gradient of 30-34% B over 20 min (where solvent is A. 0.1% TFA/H₂O and B is 0.1% TFA/CH₃CN). The ¹⁷⁷Lu-L63 that formed had an RCP of 97.8% and a retention time of ˜14.2 min on this system.

### Example L

### Preparation of ¹⁷⁷Lu-L70 for cell binding and biodistribution studies

This compound was prepared following the procedures described above, but substituting L70 (the ligand of Example II). Purification was performed using a YMC Basic C8 column (4.6×150 mm), a column temperature of 30° C. and a flow rate of 1 mL/min. with a gradient of 80% A/20% B to 75% A/25% B over 40 min., where A is citrate buffer (0.02M, pH 4.5), and B is 80% CH₃CN/20% CH₃OH. The isolated compound had an RCP of ˜100% and an HPLC retention time of 25.4 min.

### Example LI

### Preparation of ¹⁷Lu-L70 for Radiotherapy Studies

This compound was prepared as described above for L64.

### Example LII

### Preparation of ¹¹¹In-L70 for Cell Binding and Biodistribution Studies

This compound was synthesized by incubating 10 μg L70 ligand (10 μL of a 1 mg/mL solution in 0.01 N HCl), 180 μL ammonium acetate buffer (0.2M, pH 5.3), 1.1 mCi of ¹¹¹InCl₃ in 0.05N HCl (61 μL, Mallinckrodt) and 50 μL of saline at 85° C. for 30 min. Free ¹¹¹In was scavenged by adding 20 μL of a 1% Na₂EDTA*2H₂O (Aldrich) solution in water. The resulting radiochemical purity (RCP) was 86%. The radiolabeled product was separated from unlabeled ligand and other impurities by HPLC, using a Waters XTerra C18 cartridge linked to a Vydac strong anion exchange column [7.5×50 mm], a column temperature of 30° C. and a flow rate of 1 mL/min. with the gradient listed in the Table below, where A is 0.1 mM NaOH in water, pH 10.0, B is 1 g/L ammonium acetate in water, pH 6.7 and C is acetonitrile. With this system, the retention time for ¹¹¹In-L70 is 15 min while the retention time for L70 ligand is 27 to 28 min. The isolated compound had an RCP of 96%.

| Time, min | % A | % B | % C |
|---|---|---|---|
| 0-10 | 100 | | |
| 10-11 | 100-50 | | 0-50 |
| 11-21 | 50 | | 50 |
| 21-22 | 50-0 | 0-50 | 50 |
| 22-32 | | 50 | 50 |

Samples for biodistribution and cell binding studies were prepared by collecting the desired HPLC peak into 500 μL of citrate buffer (0.05 M, pH 5.3, containing 5% ascorbic acid, 1 mg/mL L-methionine and 0.2% HSA). The organic part of the collection was removed by spin vacuum for 30 min. For cell binding studies, the purified, concentrated sample was used within 30 minutes of the in vitro study. For biodistribution studies, the sample was diluted with citrate buffer (0.05 M, pH 5.3, containing 5% sodium ascorbic acid and 0.2% HSA) to a final concentration of 10.1Ci/mL within 30 minutes of the in vivo study.

### Example LIII

### In Vivo Pharmacokinetic Studies

A. Tracer Dose Biodistribution:

Low dose pharmacokinetic studies (e.g., biodistribution studies) were performed using the below-identified compounds of the invention in xenografted, PC3 tumor-bearing nude mice ([Ncr]-Foxnl<nu>). In all studies, mice were administered 100 μL of ¹⁷⁷Lu-labeled test compound at 200 μCi/kg, i.v., with a residence time of 1 and 24 h per group (n=3-4). Tissues were analyzed in an LKB 1282 CompuGamma counter with appropriate standards.

TABLE 11
Pharmacokinetic comparison at 1 and 24 h in PC3 tumor-bearing nude
mice (200 μCi/kg; values as % ID/g, except where otherwise indicated)
of ¹⁷⁷Lu-labeled compounds of this invention compared to control

| | DO3A- | | | | | | | |
|---|---|---|---|---|---|---|---|---|
| | monoamide- | | | | | | | |
| | Aoc- | | | | | | | |
| | QWAVGHLM- | | | | | | | |
| | NH₂ | | | | | | | |
| | control | | L63 | | L64 | | L70 | |
| Tissue | 1 hr | 24 hr | 1 hr | 24 hr | 1 hr | 24 hr | 1 hr | 24 hr |
| Blood | 0.44 | 0.03 | 7.54 | 0.05 | 1.87 | 0.02 | 0.33 | 0.03 |
| (% ID) | | | | | | | | |
| Liver | 0.38 | 0.04 | 12.15 | 0.20 | 2.89 | 0.21 | 0.77 | 0.10 |
| (% ID) | | | | | | | | |
| Kidneys | 7.65 | 1.03 | 7.22 | 0.84 | 10.95 | 1.45 | 6.01 | 2.31 |
| Tumor | 3.66 | 1.52 | 9.49 | 2.27 | 9.83 | 3.60 | 6.42 | 3.50 |
| Pancreas | 28.60 | 1.01 | 54.04 | 1.62 | 77.78 | 6.56 | 42.34 | 40.24 |

Whereas the distribution of radioactivity in the blood, liver and kidneys after injection of L64 and L70 is similar to that of the control compound, DO3A-monoamide-Aoc-QWAVGHLM-NH₂, wherein QWAVGHLM-NH₂ is the BBN(7-14) sequence (SEQ ID NO: 1), the uptake in the tumor is much higher at 1 and 24 h for both L64 and L70. L63 also shows high tumor uptake although with increased blood and liver values at early times. Uptake in the mouse pancreas, a normal organ known to have GRP receptors, is much higher for L64, L70 and L63 than for the control compound DO3A-monoamide-Aoc-QWAVGHLM-NH₂, wherein QWAVGHLM-NH₂ is the BBN(7-14) sequence (SEQ ID NO: 1).

B. The Effect of Mass Peptide Dose of L70 in Tumor and Normal Tissues.

The following biodistribution studies were performed in the human PC-3 nude mouse model (Tac:Cr:(NCr)-Fox1^{nu}).

Mice received one i.v. tail vein dose (0.1 mL) of the appropriate solution of ¹⁷⁷Lu-L70 to which sufficient L70 ligand was added to achieve the peptide mass recorded in the following tables. The radioactive dose of ¹⁷⁷Lu-L70 given was 10-750 μCi.

Subjects of all studies were terminated at the end of the residence interval and the organs and tissues were harvested. Radioactivity was assayed in a gamma counter. The data is expressed as percentage of the total administered radioactivity per gram of tissue (% ID/g).

| | Administered Dose (μg) | | | | | |
|---|---|---|---|---|---|---|
| Organ | 0.0025 | 0.08 | 0.22 | 0.43 | 0.64 | 0.85 |

Mouse % ID/g, 1 h

| | | | | | | |
|---|---|---|---|---|---|---|
| Blood | 0.24 | 0.77 | 0.49 | 0.85 | 0.48 | 0.71 |
| Liver | 0.21 | 0.44 | 0.27 | 0.44 | 0.26 | 0.35 |
| Kidneys | 7.09 | 5.17 | 3.46 | 4.13 | 4.75 | 5.31 |
| Tumor | 6.35 | 3.13 | 4.36 | 3.13 | 3.97 | 5.78 |
| Pancreas | 49.59 | 51.15 | 25.11 | 11.60 | 7.19 | 5.27 |
| GI | 6.38 | 4.08 | 1.41 | 1.39 | 0.94 | 0.83 |

Mouse % ID/g, 24 h

| | | | | | | |
|---|---|---|---|---|---|---|
| Organ | 0.0025 | 0.08 | 0.22 | 0.43 | 0.64 | 0.85 |
| Blood | 0.02 | 0.01 | 0.01 | 0.02 | 0.01 | 0.01 |
| Liver | 0.08 | 0.18 | 0.15 | 0.11 | 0.12 | 0.12 |
| Kidneys | 2.26 | 1.2 | 1.70 | 1.28 | 0.90 | 1.47 |
| Tumor | 3.39 | 2.59 | 2.13 | 1.64 | 0.57 | 2.85 |
| Pancreas | 35.11 | 35.18 | 19.68 | 9.55 | 4.86 | 3.29 |
| GI | 3.26 | 1.31 | 0.78 | 0.53 | 0.25 | 0.24 |

The data show that those normal organs in mice, e.g. pancreas and gastrointestinal tract, known to express the GRP receptor, demonstrate an expected mass dose effect (i.e. reduction in uptake of radioactivity with increasing mass dose), however, the tumor is unexpectedly resistant to saturation as the mass dose is increased.

A similar effect can be obtained by administering a dose of L70 ligand prior to the administration of the radioactive material. In this study, biodistribution studies were performed in the human PC-3 nude mouse model (Tac:Cr:(NCr)-Fox^{nu}) where the animals were pretreated with 1 intravenous injection of L70 ligand (0.64 μg) or buffer control at 5, 15, or 60 minutes prior to intravenous administration of ¹⁷⁷Lu-L70; mass peptide dose 0.1 μg/kg mouse. Animals were sacrificed 1 hour after administration of ¹⁷⁷Lu-L70 and tissues counted to determine if pre-treatment with L70 had any effect on biodistribution.

Down-regulation In Vivo, % Injected Dose/Organ* in PC-3 Mice,
at 1 h Post Injection of ¹⁷⁷Lu-L70

| | Time between L70 predose injection and administration of ¹⁷⁷Lu-L70 | | | |
|---|---|---|---|---|
| | No Predose (Control) | 5 min | 15 min | 60 min |
| Blood^{a} | 0.20 ± 0.04 | 0.46 ± 0.12** | 0.49 ± 0.32 | 0.22 ± 0.02 |
| Lung | 0.04 ± 0.01 | 0.09 ± 0.02** | 0.09 ± 0.04 | 0.04 ± 0.01 |
| Kidneys | 5.10 ± 2.61 | 1.73 ± 0.69* | 2.25 ± 1.30 | 3.93 ± 2.26 |
| Pancreas | 15.87 ± 4.48 | 1.22 ± 0.25**** | 2.83 ± 0.22*** | 10.14 ± 1.73 |
| Spleen | 0.10 ± 0.03 | 0.02 ± 0.01*** | 0.02 ± 0.01*** | 0.04 ± 0.01** |
| Tumor^{a} | 4.37 ± 1.42 | 5.40 ± 1.85 | 4.07 ± 1.05 | 3.21 ± 1.49 |
| G.I. | 9.50 ± 0.20 | 1.29 ± 0.77**** | 1.98 ± 0.54**** | 5.86 ± 2.46* |
| Urine | 40.59 ± 6.12 | 65.06 ± 7.25*** | 68.66 ± 12.50** | 57.88 ± 10.42* |

^{a}% injected dose/g tissue.
Level of Significance:
*p < 0.05,
**p < 0.01,
***p < 0.005,
****p < 0.001.

As before, a significant decrease in the amount of radioactivity in the pancreas was seen in animals that had been pretreated with L70 either 5 or 15 minutes prior to ¹⁷⁷Lu-L70 administration, while decreases in gastrointestinal radioactivity were seen in all L70 pre-treatment groups (5, 15, and 60 minutes). Renal effects were transient and only significantly different from control in the 5-minute predose group; however, urinary excretion differences persisted out to 60 minutes post L70 administration. The results indicate that the target tumor was unaffected by pre-treatment with L70 and demonstrated uptake of ¹⁷⁷Lu-L70 under all predose conditions. Combined, these results show a beneficial effect of pre-dosing or co-dosing with compounds of the invention.

### Example LIV

### Receptor Subtype Specificity

Currently, four mammalian members of the GRP receptor family are known: the GRP-preferring receptor (GRP-R), neuromedin-B preferring receptor (NMB-R), the bombesin receptor subtype 3 (BB3-R) and the bombesin receptor subtype 4 (BB4-R). The receptor subtype specificity of ¹⁷⁷Lu-L70 was investigated. The results indicate ¹⁷⁷Lu-L70 binds specifically to GRP-R and NMB-R, and has little affinity for BB3-R. The subtype specificity of the Lutetium complex of L70 (here, ¹⁷⁷Lu-L70) (prepared as described supra) was determined by in vitro receptor autoradiography using the procedure described in Reubi et al., “Bombesin Receptor Subtypes in Human Cancers: Detection with the Universal Radioligand ¹²⁵I-[D-Tyr⁶, beta-Ala, Phe¹³, Nle¹⁴]”, Clin.Cancer Res. 8:1139-1146 (2002) and tissue samples that had been previously found to express only one subtype of GRP receptor, as well as non-neoplastic tissues including normal pancreas and colon, as well as chronic pancreatitis (shown below in Table 12a). Human ileal carcinoid tissue was used as a source for NMB-R, human prostate carcinoma for GRP-R and human bronchial carcinoid for BB3-R subtype receptors. For comparison, receptor autoradiography was also performed with other bombesin radioligands, such as ¹²⁵I-Tyr⁴-bombesin or a compound known as the Universal ligand, ¹²⁵I-[DTyr⁶, βAla¹¹, Phe¹³, Nle¹⁴]-BBN(6-14), which binds to all three subsets of GRP-R, on adjacent tissue sections. For further discussion, see Fleischmann et al., “Bombesin Receptors in Distinct Tissue Compartments of Human Pancreatic Diseases,” Lab. Invest. 80:1807-1817 (2000); Markwalder et al., “Gastrin-Releasing Peptide Receptors in the Human Prostate: Relation to Neoplastic Transformation,” Cancer Res. 59:1152-1159 (1999); Gugger et al., “GRP Receptors in Non-Neoplastic and Neoplastic Human Breast,” Am. J. Pathol. 155:2067-2076 (1999).

TABLE 12A
Detection of bombesin receptor subtypes in various human tissues
using different radioligands.

| | | | | | Receptor | | |
|---|---|---|---|---|---|---|---|
| | | Receptor | | | autoradiography using | | |
| | | autoradiography | | | standard BN | | |
| | | using ¹⁷⁷Lu-L70 | | | radioligands* | | |
| Tumor | n | GRP-R | NMB-R | BB3 | GRP-R | NMB-R | BB3 |
| Mammary | 8 | 8/8 | 0/8 | 0/8 | 8/8 | 0/8 | 0/8 |
| Ca | | | | | | | |
| Prostate Ca | 4 | 4/4 | 0/4 | 0/4 | 4/4 | 0/4 | 0/4 |
| Renal Ca | 6 | 5/6 | 0/6 | 0/6 | 4/6 | 0/6 | 0/6 |
| Ileal | 8 | 0/8 | 8/8 | 0/8 | 0/8 | 8/8 | 0/8 |
| carcinoid | | | | | | | |
| Bronchial | 6 | 2/6 | 0/6 | 0/6 | 2/6 | 0/6 | 6/6 |
| carcinoid | | (weak) | | | (weak) | | |
| Colon Ca | | | | | | | |
| tumor | 7 | 3/7 | 0/7 | 0/7 | 3/7 | 0/7 | 0/7 |
| | | (weak) | | | (weak) | | |
| smooth | 7 | 7/7 | 0/7 | 0/7 | 7/7 | 0/7 | 0/7 |
| muscle | | | | | | | |
| Pancreas Ca | 4 | 0/4 | 0/4 | 0/4 | 0/4 | 0/4 | 0/4 |
| Chronic | 5 | 5/5 | 0/5 | 0/5 | 5/5 | 0/5 | 0/5 |
| pancreatitis | | | | | | | |
| (acini) | | | | | | | |
| Human | 7 | 1/7 | 0/7 | 0/7 | 0/7 | 0/7 | 0/7 |
| pancreas | | (weak) | | | | | |
| (acini) | | | | | | | |
| Mouse | 4 | 4/4 | 0/4 | 0/4 | 4/4 | 0/4 | 0/4 |
| pancreas | | | | | | | |
| (acini) | | | | | | | |

*¹²⁵I-[DTyr⁶, βAla¹¹, Phe¹³, Nle¹⁴]-BBN(6-14) and ¹²⁵I-Tyr⁴-BBN.

As seen from Table 12a, all GRP-R-expressing tumors such as prostatic, mammary and renal cell carcinomas, identified as such with established radioligands, were also visualized in vitro with ¹⁷⁷Lu-L70. Due to a better sensitivity, selected tumors with low levels of GRP-R could be identified with ¹⁷⁷Lu-L70, but not with ¹²⁵I-Tyr⁴-BBN, as shown in Table 12a. All NMB-R-expressing tumors identified with established radioligands were also visualized with ¹⁷⁷Lu-L70. Conversely, none of the BB3 tumors were detected with ¹⁷⁷Lu-L70. One should not make any conclusion on the natural incidence of the receptor expression in the various types of tumors listed in Table 12a, as the tested cases were chosen as receptor-positive in the majority of cases, with only a few selected negative controls. The normal human pancreas is not labeled with ¹⁷⁷Lu-L70, whereas the mouse pancreas is strongly labeled under identical conditions. Although the normal pancreas is a very rapidly degradable tissue and one can never completely exclude degradation of protein, including receptors, factors suggesting that the human pancreas data are truly negative include the positive control of the mouse pancreas under similar condition and the strongly labeled BB3 found in the islets of the respective human pancreas, which represent a positive control for the quality of the investigated human pancreas. Furthermore, the detection of GRP-R in pancreatic tissues that are pathologically altered (chronic pancreatitis) indicates that GRP-R, when present, can be identified under the chosen experimental conditions in this tissue. In fact, ¹⁷⁷Lu-L70 identifies these GRP-R in chronic pancreatitis with greater sensitivity than ¹²⁵I-Tyr⁴-BBN. While none of the pancreatic cancers had measurable amounts of GRP-R, a few colon carcinomas showed a low density of heterogeneously distributed GRP receptors measured with ¹⁷⁷Lu-L70 (Table 12a). It should further be noticed that the smooth muscles of the colon express GRP-R and were detected in vitro with ¹⁷⁷Lu-L70 as well as with the established bombesin ligands.

TABLE 12B
Binding affinity of ¹⁷⁵Lu-L70 to the 3 bombesin receptor subtypes
expressed in human cancers. Data are expressed as IC₅₀ in nM
(mean ± SEM. n = number of experiments in parentheses).

| Compound | D. NMB-R | E. GRP-R | BB3 |
|---|---|---|---|
| Universal | 0.8 ± 0.1 (3) | 0.7 ± 0.1 (3) | 1.1 ± 0.1 (3) |
| ligand | | | |
| ¹⁷⁵Lu-L70 | 0.9 ± 0.1 (4) | 0.8 ± 0.1 (5) | >1,000 (3) |

As shown in Table 12b, the cold labeled ¹⁷⁵Lu-L70 had a very high affinity for human GRP and NMB receptors expressed in human tissues while it had only low affinity for BB3 receptors. These experiments used ¹²⁵I-[DTyr⁶, βAla¹¹, Phe¹³, Nle¹⁴]-BBN(6-14) as radiotracer. Using the ¹⁷⁷Lu-labeled L70 as radiotracer, the above mentioned data are hereby confirmed and extended. All GRP-R-expressing human cancers were very strongly labeled with ¹⁷⁷Lu-L70. The same was true for all NMB-R-positive tumors. Conversely, tumors with BB3 were not visualized. The sensitivity of ¹⁷⁷Lu-L70 seems better than that of ¹²⁵I-Tyr⁴-BBN or the ¹²⁵I-labeled universal bombesin analog. Therefore, a few tumors expressing a low density of GRP-R can be readily identified with ¹⁷⁷Lu-L70, while they are not positive with ¹²⁵I-Tyr⁴-BBN. The binding characteristics of ¹⁷⁷Lu-L70 could also be confirmed in non-neoplastic tissues. While the mouse pancreas, as control, was shown to express a very high density of GRP-R, the normal human pancreatic acini were devoid of GRP-R. However, in conditions of chronic pancreatitis GRP-R could be identified in acini, as reported previously in Fleischmann et al., “Bombesin Receptors in Distinct Tissue Compartments of Human Pancreatic Diseases”, Lab. Invest. 80:1807-1817 (2000) and tissue, again with better sensitivity by using ¹⁷⁷Lu-L70 than by using ¹²⁵I-Tyr⁴-BBN. Conversely, the BB₃-expressing islets were not detected with ¹⁷⁷Lu-L70, while they were strongly labeled with the universal ligand, as reported previously in Fleischmann et al., “Bombesin Receptors in Distinct Tissue Compartments of Human Pancreatic Diseases”, Lab. Invest. 80:1807-1817 (2000). While a minority of colon carcinomas had GRP-R, usually in very low density and heterogeneously distributed, the normal colonic smooth muscles expressed a high density of GRP-R.

The results in Tables 12a and 12b indicate that Lu labeled L70 derivatives are expected to bind well to human prostate carcinoma, which primarily expresses GRP-R. They also indicate that Lu labeled L70 derivatives are not expected to bind well to normal human pancreas (which primarily expresses the BB3-R receptor), or to cancers which primarily express the BB3-R receptor subtype.

### Example LV

### Radiotherapy Studies

A. Efficacy Studies:

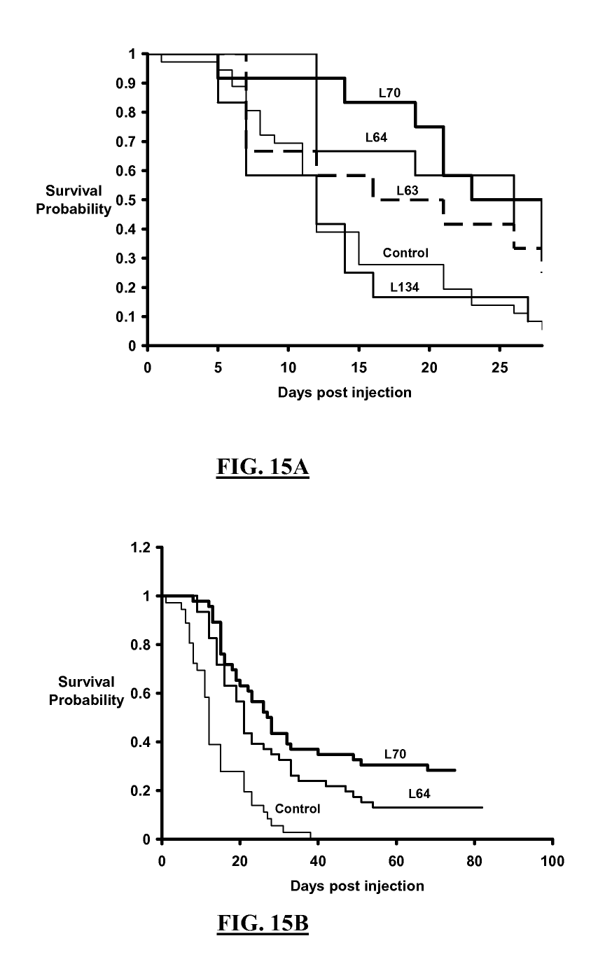

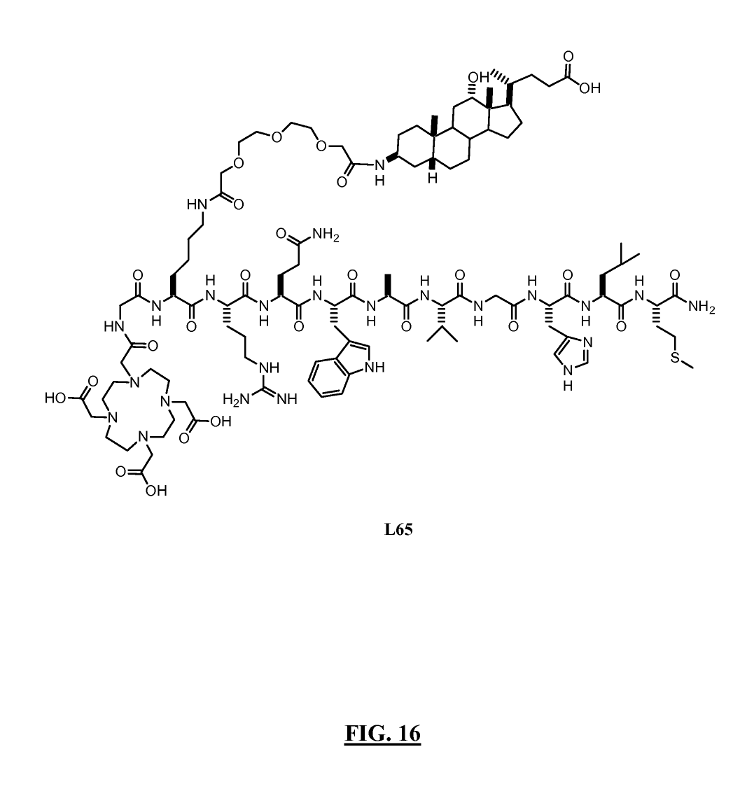

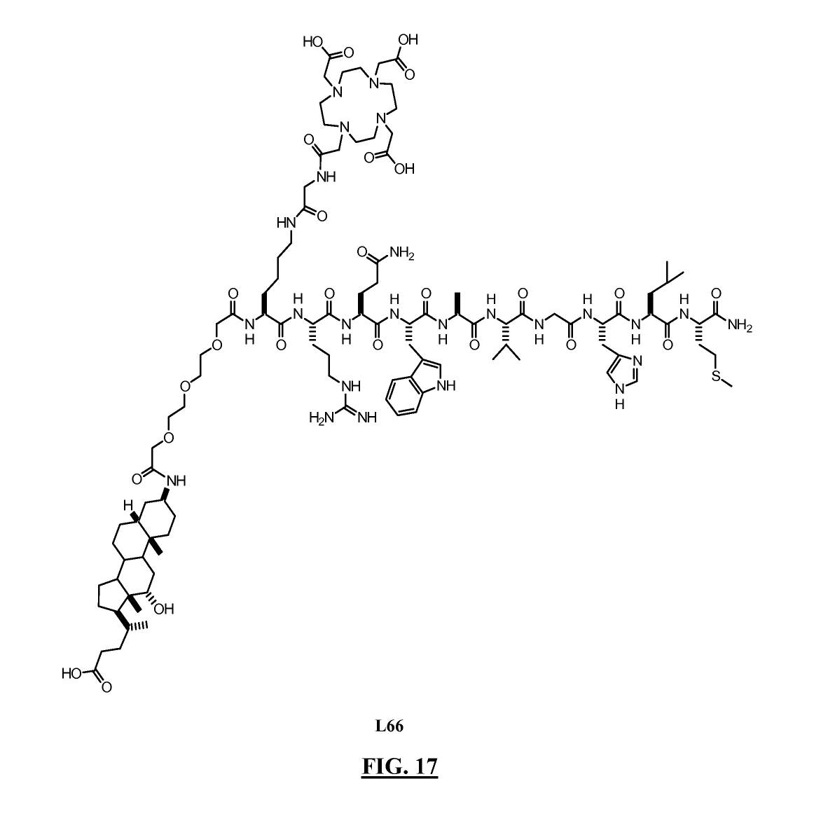

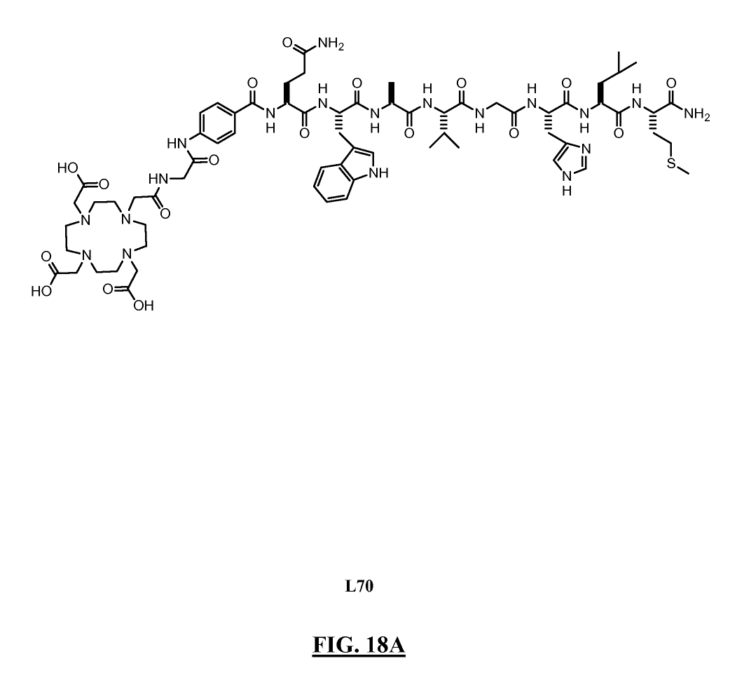

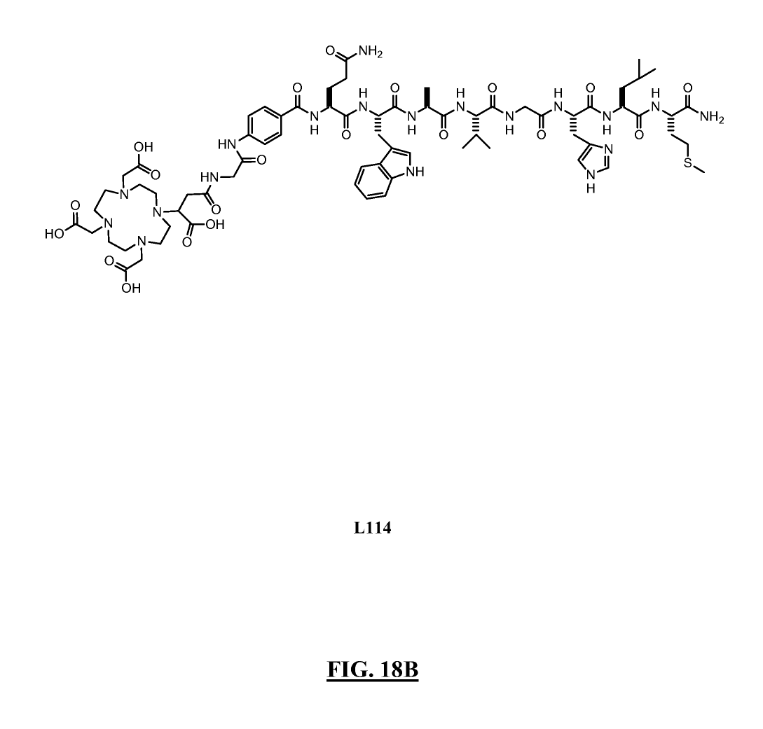

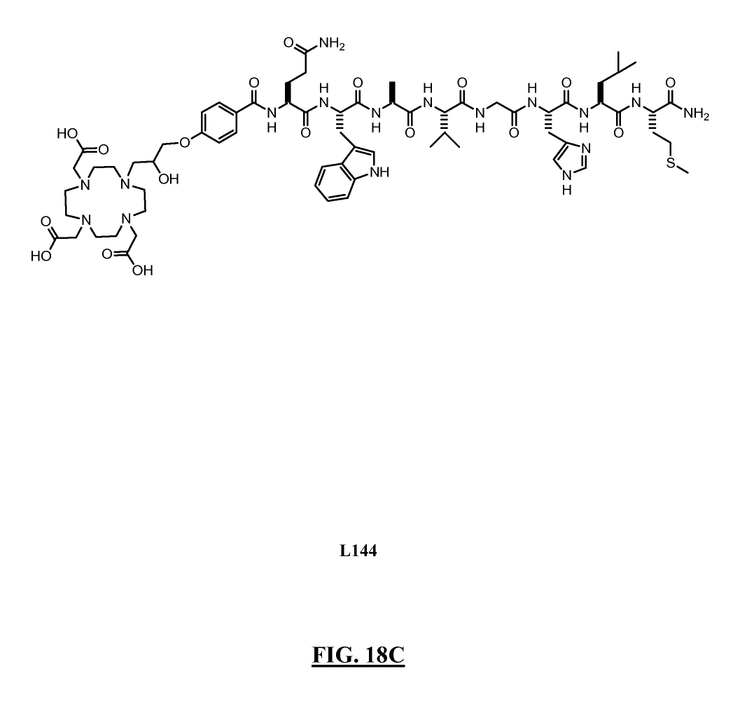

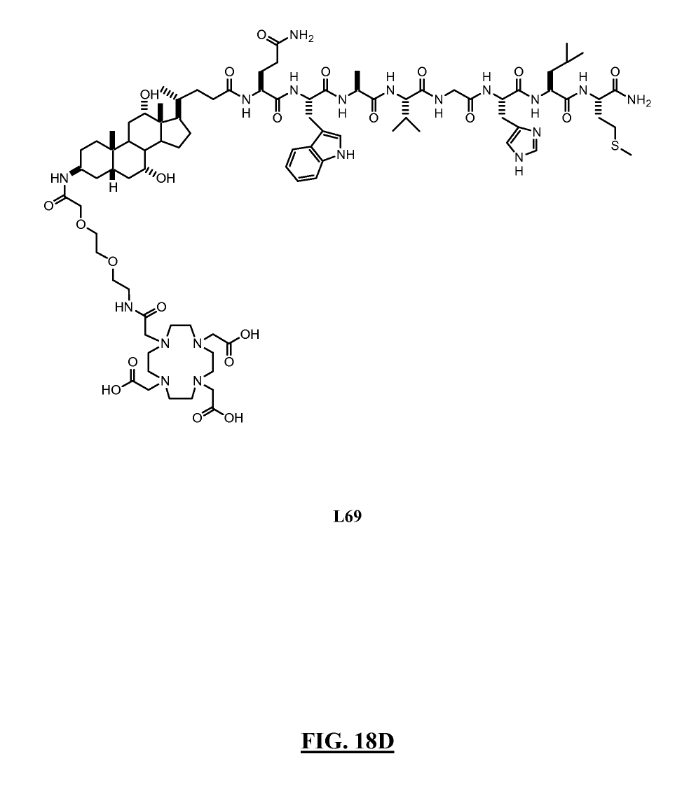

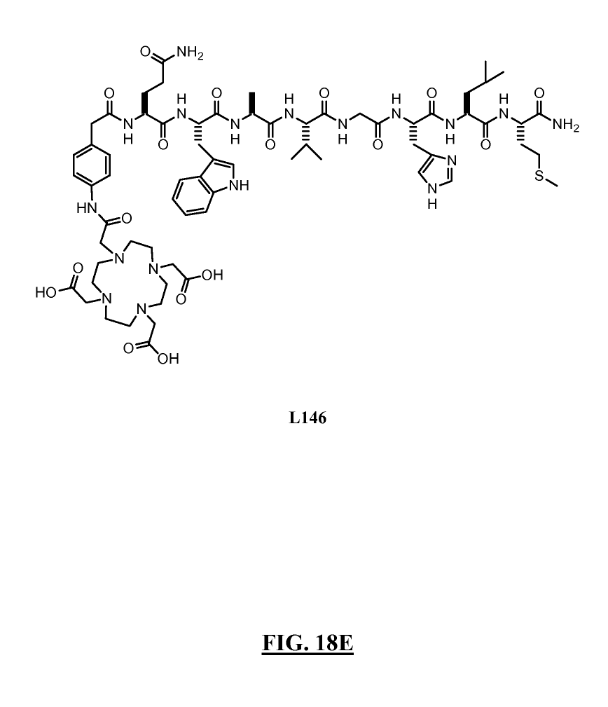

Radiotherapy studies were performed using the PC3 tumor-bearing nude mouse model. In Short Term Efficacy Studies, ¹⁷⁷Lu labeled compounds of the invention L64, L70, L63 and the treatment control compound DO3A-monoamide-Aoc-QWAVGHLM-NH₂ were compared to an untreated control group. (n=12 for each treatment group for up to days, and n=36 for the pooled untreated control group for up to 31 days). For all efficacy studies, mice were administered 100 μL of ¹⁷⁷Lu-labeled compound of the invention at 30 mCi/kg, i.v, or s.c. under sterile conditions. The subjects were housed in a barrier environment for the duration of the study. Body weight and tumor size (by caliper measurement) were collected on each subject 3 times per week for the duration of the study. Criteria for early termination included: death; loss of total body weight (TBW) equal to or greater than 20%; tumor size equal to or greater than 2 cm³. Results of the Short Term Efficacy Study are displayed in FIG. 15A. These results show that animals treated with L70, L64 or L63 have increased survival over the control animals given no treatment and over those animals given the same dose of the DO3A-monoamide-Aoc-QWAVGHLM-NH₂ control.

Long Term Efficacy Studies were performed with L64 and L70 using the same dose as before but using more animals per compound (n=46) and following them for up to 120 days. The results of the Long Term Efficacy Study are displayed in FIG. 15B. Relative to the same controls as before (n=36), both L64 and L70 treatment gave significantly increased survival (p<0.0001) with L70 being better than L64, although not statistically different from each other (p<0.067).

### Example LVI

### Alternative Preparation of L64 and L70 Using Segment Coupling

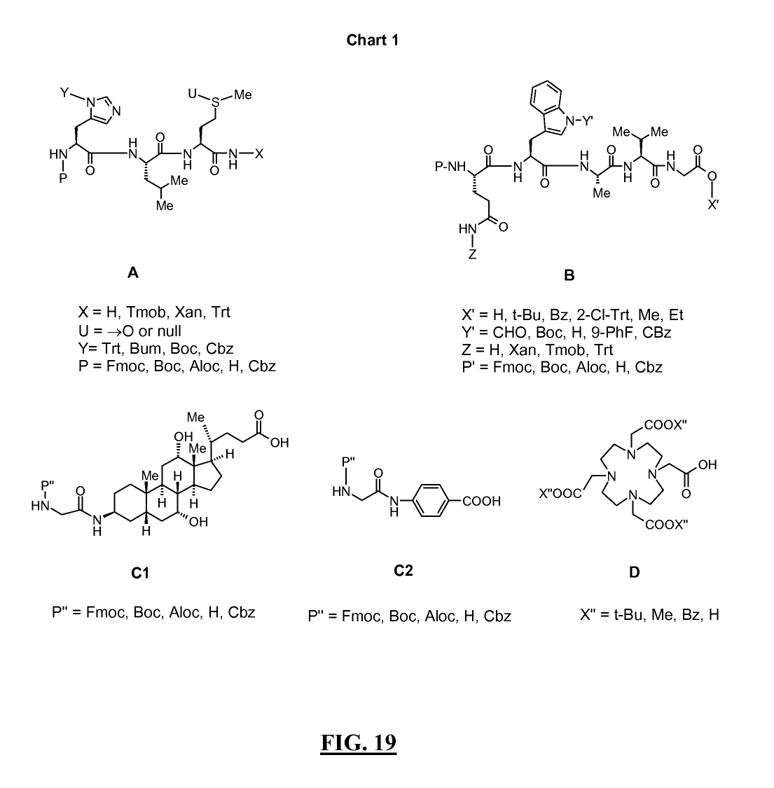

Compounds L64 and L70 can be prepared employing the collection of intermediates generally represented by A-D (FIG. 19), which themselves are prepared by standard methods known in the art of solid and solution phase peptide synthesis (Synthetic Peptides—A User's Guide 1992, Grant, G., Ed. WH. Freeman Co., NY, Chap 3 and Chap 4 pp 77-258; Chan, W. C. and White, P. D. Basic Procedures in Fmoc Solid Phase Peptide Synthesis—A Practical Approach 2002, Chan, W. C. and White, P. D. Eds Oxford University Press, New York, Chap. 3 pp 41-76; Barlos, K. and Gatos, G. Convergent Peptide Synthesis in Fmoc Solid Phase Peptide Synthesis—A Practical Approach 2002, Chan, W. C. and White, P.D. Eds Oxford University Press, New York, Chap. 9 pp 216-228) which are incorporated herein by reference.

These methods include Aloc, Boc, Fmoc or benzyloxycarbonyl-based peptide synthesis strategies or judiciously chosen combinations of those methods on solid phase or in solution. The intermediates to be employed for a given step are chosen based on the selection of appropriate protecting groups for each position in the molecule, which may be selected from the list of groups shown in FIG. 1. Those of ordinary skill in the art will also understand that intermediates, compatible with peptide synthesis methodology, comprised of alternative protecting groups can also be employed and that the listed options for protecting groups shown above serves as illustrative and not inclusive, and that such alternatives are well known in the art.

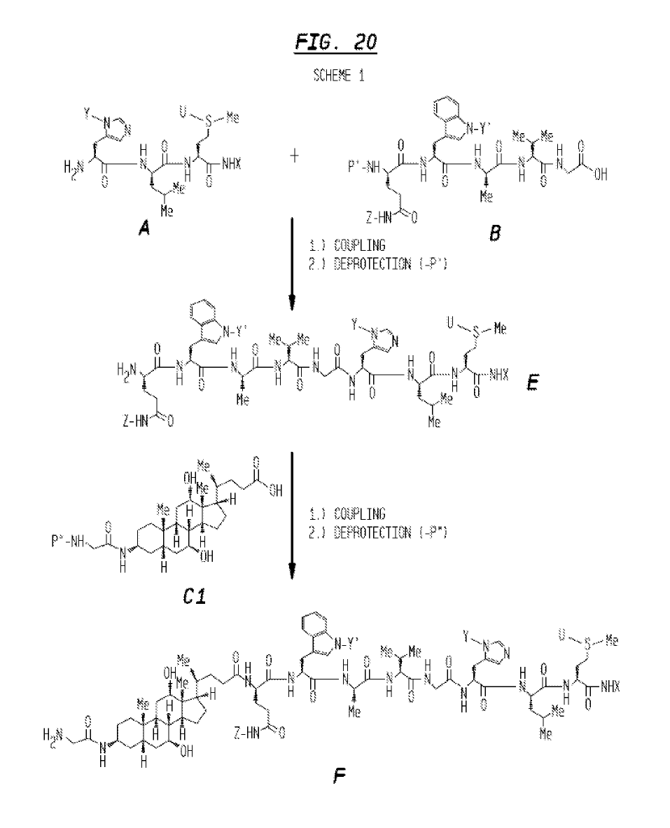

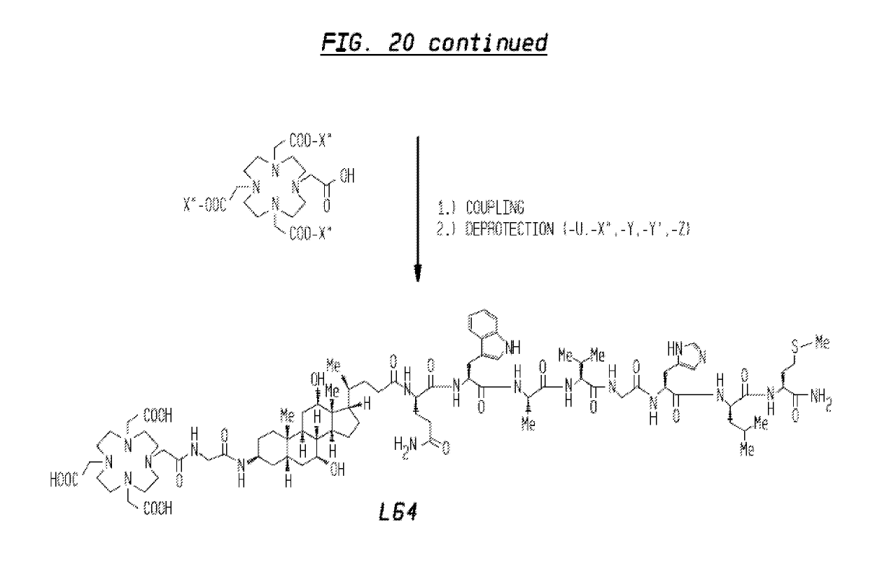

This is amply illustrated in FIG. 20 which outlines the approach. Substitution of the intermediate C2 in place of C1 shown in the synthesis of L64, provides L70 when the same synthetic strategies are applied.

### Example LVII

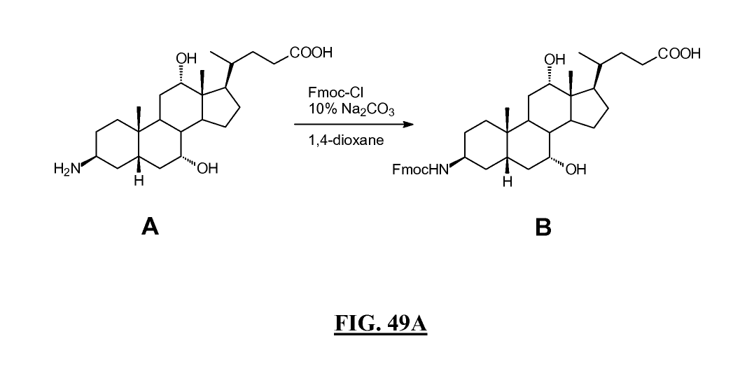

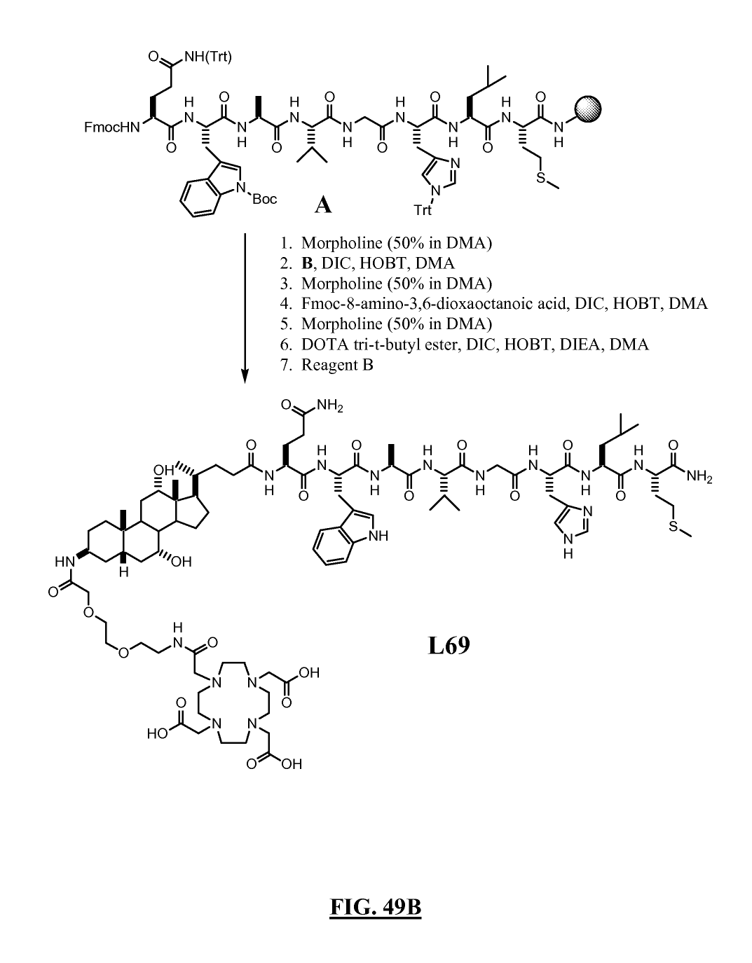

### FIGS. 49A and 49B

### Synthesis of L69

Summary: Reaction of (3β,5β,7α,12α)-3-amino-7,12-dihydroxycholan-24-oic acid A with Fmoc-Cl gave intermediate B. Rink amide resin functionalised with the octapeptide Gln-Trp-Ala-Val-Gly-His-Leu-Met-NH₂ (BBN[7-14]) (SEQ ID NO: 1) (A), was sequentially reacted with B, Fmoc-8-amino-3,6-dioxaoctanoic acid and DOTA tri-t-butyl ester. After cleavage and deprotection with Reagent B the crude was purified by preparative HPLC to give L230. Overall yield: 4.2%.

### A. (3β,5β,7α,12α)-3-(9H-Fluoren-9-ylmethoxy)amino-7,12-dihydroxycholan-24-oic acid, B (FIG. 49A)

A solution of 9-fluorenylmethoxycarbonyl chloride (1.4 g; 5.4 mmol) in 1,4-dioxane (18 mL) was added dropwise to a suspension of (3β,5β,7α,12α)-3-amino-7,12-dihydroxycholan-24-oic acid A (2.0 g; 4.9 mmol) (3) in 10% aq. Na₂CO₃ (30 mL) and 1,4-dioxane (18 mL) stirred at 0° C. After 6 h stirring at room temperature H₂O (100 mL) was added, the aqueous phase washed with Et₂O (2×90 mL) and then 2 M HCl (15 mL) was added (final pH: 1.5). The precipitated solid was filtered, washed with H₂O (3×100 mL), vacuum dried and then purified by flash chromatography to give B as a white solid (2.2 g; 3.5 mmol). Yield 71%.

### B N-[3β,5β,7α,12α)-3-[[[2-[2-[[[4,7,10-Tris(carboxymethyl)-1,4,7,10-tetraazacyclododec-1-yl]acetyl]amino]ethoxy]ethoxy]acetyl]amino]-7,12-dihydroxy-24-oxocholan-24-yl]-L-glutaminyl-L-tryptophyl-L-alanyl-L-valyl-glycyl-L-histidvl-L-leucyl-L-methioninamide, L69 (FIG. 49B)

Resin A (0.5 g; 0.3 mmol) was shaken in a solid phase peptide synthesis vessel with 50% morpholine in DMA (7 mL) for 10 min, the solution filtered and fresh 50% morpholine in DMA (7 mL) was added. The suspension was stirred for another 20 min then the solution was filtered and the resin washed with DMA (5×7 mL). (3β,5β,5β,7α,12α)-3-(9H-Fluoren-9-ylmethoxy)amino-7,12-dihydroxycholan-24-oic acid B (0.75 g; 1.2 mmol), N-hydroxybenzotriazole (HOBt) (0.18 g; 1.2 mmol), N,N′-diisopropylcarbodiimide (DIC) (0.19 mL; 1.2 mmol) and DMA (7 mL) were added to the resin, the mixture shaken for 24 h at room temperature, emptied and the resin washed with DMA (5×7 mL). The resin was then shaken with 50% morpholine in DMA (7 mL) for 10 min, the solution emptied, fresh 50% morpholine in DMA (7 mL) was added and the mixture shaken for another 20 min. The solution was emptied and the resin washed with DMA (5×7 mL). Fmoc-8-amino-3,6-dioxaoctanoic acid (0.79 g; 1.2 mmol), HOBt (0.18 g; 1.2 mmol), DIC (0.19 mL; 1.2 mmol) and DMA (7 mL) were added to the resin. The mixture was shaken for 3 h at room temperature, emptied and the resin washed with DMA (5×7 mL). The resin was then shaken with 50% morpholine in DMA (7 mL) for 10 min, the solution filtered, fresh 50% morpholine in DMA (7 mL) was added and the mixture shaken for another 20 min. The solution was filtered and the resin washed with DMA (5×7 mL) 1,4,7,10-Tetraazacyclododecane-1,4,7,10-tetraacetic acid tris(1,1-dimethylethyl) ester adduct with NaCl (0.79 g; 1.2 mmol), HOBt (0.18 g; 1.2 mmol), DIC (0.19 mL: 1.2 mmol), N-ethyldiisopropylamine (0.40 mL; 2.4 mmol) and DMA (7 mL) were added to the resin. The mixture was shaken for 24 h at room temperature, filtered and the resin washed with DMA (5×7 mL), CH₂Cl₂ (5×7 mL) and vacuum dried. The resin was shaken in a flask with Reagent B (25 mL) (2) for 4.5 h. The resin was filtered and the solution was evaporated under reduced pressure to afford an oily crude that after treatment with Et₂O (20 mL) gave a precipitate. The precipitate was collected by centrifugation and washed with Et₂O (3×20 mL) to give a solid (248 mg) which was analysed by HPLC. An amount of crude (50 mg) was purified by preparative HPLC. The fractions containing the product were lyophilized to give L69 (6.5 mg; 3.5×10⁻³ mmol) (FIG. 49B) as a white solid. Yield 5.8%.

### Example LVIII

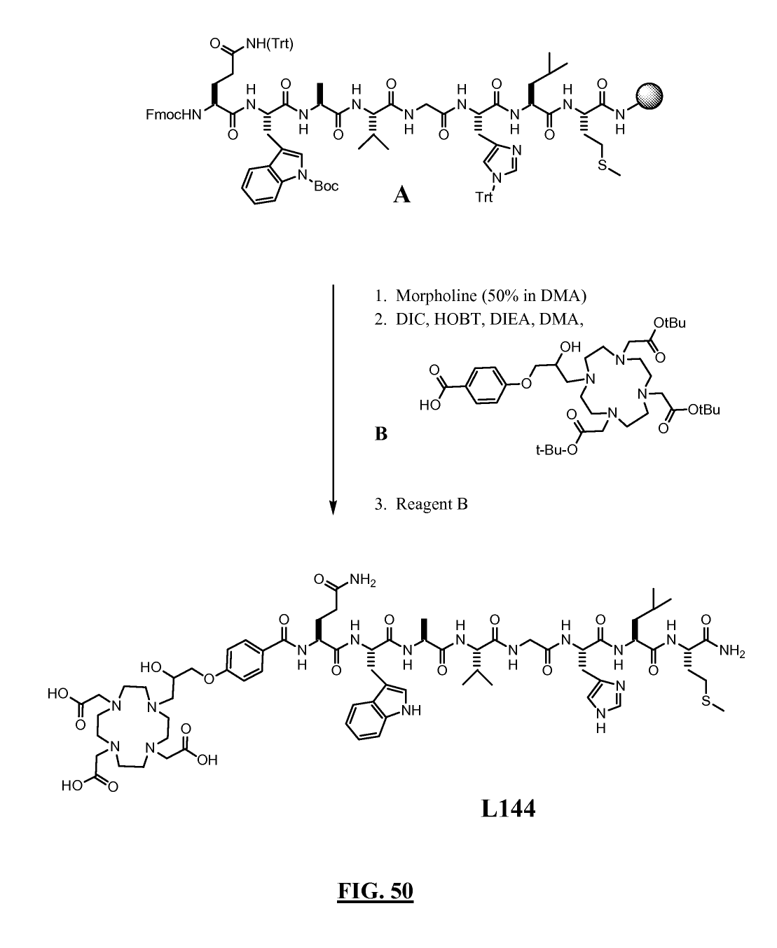

### FIG. 50

### Synthesis of L144

Summary: Rink amide resin functionalised with the octapeptide Gln-Trp-Ala-Val-Gly-His-Leu-Met-NH₂ (BBN[7-14]) (SEQ ID NO: 1) (A) was reacted with 4-[2-hydroxy-3-[4,7,10-tris[2-(1,1-dimethylethoxy)-2-oxoethyl]-1,4,7,10-tetrazacyclododec-1-yl]propoxy]benzoic acid. After cleavage and deprotection with Reagent B (2) the crude was purified by preparative HPLC to give L144. Overall yield: 12%.

### A. N-[4-[2-Hydroxy-3-[4,7,10-tris(carboxymethyl)-1,4,7,10-tetraazacyclododec-1-yl]propoxy]benzoyl]-L-glutaminyl-L-tryptophyl-L-alanyl-L-valyl-glycyl-L-histidvl-L-leucyl-L-methioninamide, L144 (FIG. 50)

Resin A (0.4 g; 0.24 mmol) was shaken in a solid phase peptide synthesis vessel with 50% morpholine in DMA (7 mL) for 10 min, the solution filtered and fresh 50% morpholine in DMA (7 mL) was added. The suspension was stirred for another 20 min then the solution was filtered and the resin washed with DMA (5×7 mL). 4-[2-Hydroxy-3-[4,7,10-tris [2-(1,1-dimethylethoxy)-2-oxoethyl]-1,4,7,10-tetrazacyclododec-1-yl]propoxy]benzoic acid B (0.5 g; 0.7 mmol), HOBt (0.11 g; 0.7 mmol), DIC (0.11 mL; 0.7 mmol)), N-ethyldiisopropylamine (0.24 mL; 1.4 mmol) and DMA (7 mL) were added to the resin. The mixture was shaken for 24 h at room temperature, emptied and the resin washed with DMA (5×7 mL), CH₂Cl₂ (5×7 mL) and vacuum dried. The resin was shaken in a flask with Reagent B (25 mL) (2) for 4.5 h. The resin was filtered and the solution was evaporated under reduced pressure to afford an oily crude that after treatment with Et₂O (20 mL) gave a precipitate. The precipitate was collected by centrifugation and washed with Et₂O (3×20 mL) to give a solid (240 mg) which was analysed by HPLC. An amount of crude (60 mg) was purified by preparative HPLC. The fractions containing the product were lyophilized to give L144 (10.5 mg; 7.2×10⁻³ mmol) as a white solid. Yield 12%.

### Example LIX

### Preparation of L300 and ¹⁷⁷Lu-L300

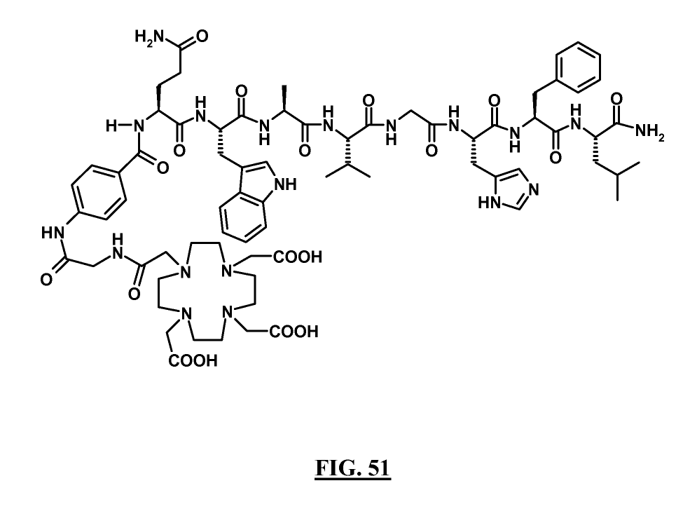

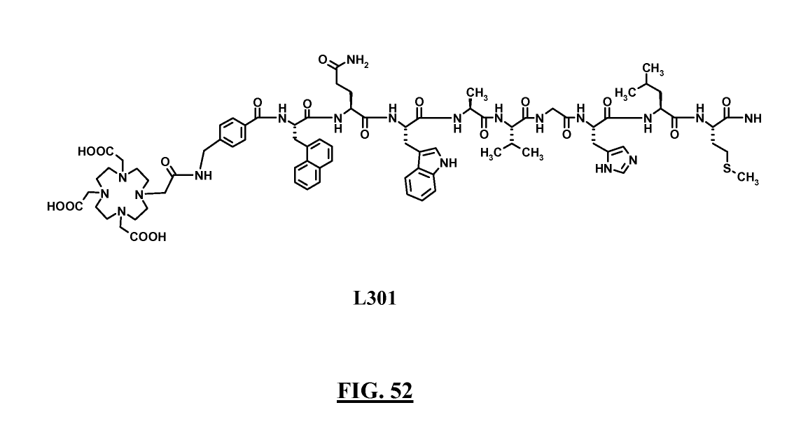

From 0.2 g of Rink amide Novagel resin (0.63 mmol/g, 0.126 mmol), L300 (0.033 g, 17%) was obtained after preparative column chromatography. The retention time was 6.66 minutes. The molecular formula is C₇₂H₉₉N₁₉O₁₈. The calculated molecular weight is 1518.71; 1519.6 observed. The sequence is DO3A-Gly-Abz-4-Gln-Trp-Ala-Val-Gly-His-Phe-Leu-NH₂, wherein Gln-Trp-Ala-Val-Gly-His-Phe-Leu-NH₂ (i.e., QWAVGHFL-NH₂) is SEQ ID NO: 22. The structure of L300 is shown in FIG. 51.

L300 (13.9 μg in 13.9 μL of 0.2M pH 4.8 sodium acetate buffer) was mixed with 150 μL of 0.2M pH 4.8 sodium acetate buffer and 4 μL of ¹⁷⁷LuCl₃ (1.136 mCi, Missouri Research Reactor). After 10 min at 100° C., the radiochemical purity (RCP) was 95%. The product was purified on a Vydac C18 peptide column (4.6×250 mm, 5 um pore size) eluted at a flow rate of 1 mL/min using an aqueous/organic gradient of 0.1% TFA in water (A) and 0.085% TFA in acetonitrile (B). The following gradient was used: isocratic 22% B for 30 min, to 60% B in 5 min, hold at 60% B for 5 min. The compound, which eluted at a retention time of 18.8 min., was collected into 1 mL of an 0.8% human serum albumin solution that was prepared by adding HSA to a 9:1 mixture of normal saline and Ascorbic Acid, Injection. Acetonitrile was removed using a Speed Vacuum (Savant). After purification, the compound had an RCP of 100%.

### Example LX

### Characterization of Linker Specificity in Relation to GRP Receptor Subtypes

Two cell lines, C6, an NMB-R expressing rodent glioblastoma cell line and PC3, a GRP-R expressing human prostate cancer cell line, were used in this assay. The affinity of various unlabeled compounds for each receptor subtype (NMB-R and GRP-R) was determined indirectly by measuring its ability to compete with the binding of ¹²⁵I-NMB or ¹²⁵I-BBN to its corresponding receptors in C6 and PC3 cells.

A. Materials and Methods:

1. Cell Culture:

C6 cells were obtained from ATCC(CCL-107) and cultured in F12K media (ATCC) supplemented with 2 mM L-glutamine, 1.5 g/L Sodium bicarbonate, 15% horse serum and 2.5% FBS. Cells for the assays were plated at a concentration of 9.6×10⁴/well in 48 well poly-lysine coated plates (Beckton Dickinson Biocoat). PC3 were obtained from ATCC(CRL-1435) and cultured in RPMI 1640 (ATCC) supplemented with 2 mM L-glutamine, 1.5 g/L Sodium bicarbonate, 10 mM HEPES and 10% FBS. Both cultures were maintained in a humidified atmosphere containing 5% CO₂/95% air at 37° C. PC3 cells for the assays were plated at a concentration of 2.0×10⁴ cells/well in 96-well white/clear bottom plates (Falcon Optilux-I). Plates were used for the assays on day 2 of the post-plating.

2. Binding Buffer, and Radio-Ligands:

RPMI 1640 (ATCC) containing 25 mM HEPES, 0.2% BSA fraction V, 1.0 mM AEBSF (CAS #3087-99-7) and 0.1% Bacitracin (CAS #1405-87-4), pH 7.4. Custom made ¹²⁵I-[Tyr°]NMB, >2.0 Ci/mole (Amersham Life Science) [¹²⁵I-NMB] and commercially available ¹²⁵I-[Tyr⁴]BBN, >2.0 Ci/mole (Perkin Elmer Life Science) [¹²⁵I-BBN] were used as radioligands.

B. In Vitro Assay:

Using a 48-well plate assay system (for C6 study) competition experiments were performed using ¹²⁵I-NMB. All of the PC3 studies were performed as described in Example XLIII using ¹²⁵I-BBN. Selection of compounds for the assay was based on linker subtype. Results are shown in Table 13.

TABLE 13
Number of selected compounds for the assay and their linkers

| | NUMBER OF |
|---|---|
| LINKER TYPE | COMPOUNDS |
| Neutral, Basic or combination of neutral, basic & acidic | 8 |
| Linear aliphatic (ω-aminoalkanoic & ω-aminoalkoxynoic | 4 |
| acid | |
| Bile acids (cholic acids) | 3 |
| Substituted alanine (cycloalkyl, aromatic and | 5 |
| heteroaromatic) | |
| Aromatic (aminobenzoic acid and aminoalkyl benzoic | 12 |
| acid, biphenyl) | |
| Cyclic non-aromatic | 5 |
| Heterocyclic (aromatic and non-aromatic) | 5 |
| Miscellaneous (DOTA-NMB, DOTA-G-Abz4-NMB, | 6 |
| DOTA-Abz4-G-NMB, BBN₇₋₁₄, BBN₈₋₁₄, | |
| DOTA-BBN₇₋₁₄) | |

The binding parameters obtained from the studies were analyzed using a one-site competition non-linear regression analysis with GraphPad Prism. The relative affinity of various compounds for NMB-R in C6 cells were compared with those obtained using commercially available [Tyr⁴]-BBN and [Tyro]-NMB. To distinguish the GRP-R preferring compounds from NMB-R plus GRP-R preferring compounds, IC₅₀ values obtained for each compound were compared with those obtained from [Tyr⁴-BBN with ¹²⁵I-NMB on C6 cells. The cut off point between the two classes of compounds was taken as 10× the IC₅₀ of [Tyr⁴]-BBN. Among the compounds tested, 8 compounds preferentially bind to GRP-R (as shown in Table 14) while 32 compounds bind to both GRP-R and NMB-R with similar affinity, and two show preference for NMB-R.

TABLE 14
The IC₅₀ values obtained from competition experiments
using ¹²⁵I-NMB and ¹²⁵I-BBN

| | | IC₅₀ (nM) | | | GRP-R |
|---|---|---|---|---|---|
| | | ¹²⁵I-BBN/ | ¹²⁵I-NMB/ | | and |
| L # | COMPOUND | PC3 | C6 | GRP-R | NMB-R |
| na | N,N-dimethylglycine-Ser- | 10 | 10.4 | — | yes |
| | Cys(Acm)-Gly-SS- | | | | |
| | QWAVGHLM-NH₂* | | | | |
| na | N,N-dimethylglycine-Ser- | 25 | 7.9 | — | yes |
| | Cys(Acm)-Gly-G- | | | | |
| | QWAVGHLM-NH₂* | | | | |
| na | N,N-dimethylglycine-Ser- | 48 | 20.2 | — | yes |
| | Cys(Acm)-Gly-GG- | | | | |
| | QWAVGHLM-NH₂* | | | | |
| na | N,N-dimethylglycine-Ser- | 13 | 6.4 | — | yes |
| | Cys(Acm)-Gly-KK- | | | | |
| | QWAVGHLM-NH₂* | | | | |
| na | N,N-dimethylglycine-Ser- | 2 | 2.2 | — | yes |
| | Cys(Acm)-Gly-SK- | | | | |
| | QWAVGHLM-NH₂* | | | | |
| na | N,N-dimethylglycine-Ser- | 1.9 | 2.0 | — | yes |
| | Cys(Acm)-Gly-SR- | | | | |
| | QWAVGHLM-NH₂* | | | | |
| na | N,N-dimethylglycine-Ser- | 7.5 | 24.1 | yes | — |
| | Cys(Acm)-Gly-KS- | | | | |
| | QWAVGHLM-NH₂* | | | | |
| na | N,N-dimethylglycine-Ser- | 32 | 60.0 | yes | — |
| | Cys(Acm)-Gly-KE- | | | | |
| | QWAVGHLM-NH₂* | | | | |
| na | DO3A-monoamide-Aoc- | 3.4 | 3.1 | — | yes |
| | QWAVGHLM-NH₂* | | | | |
| na | DO3A-monoamide-Apa3- | 36 | 18.9 | — | yes |
| | QWAVGHLM-NH₂* | | | | |
| na | DO3A-monoamide-Abu4- | 19.8 | 5.2 | — | yes |
| | QWAVGHLM-NH₂* | | | | |
| L3 | N,N-dimethylglycine-Ser- | 70 | 33 | — | yes |
| | Cys(Acm)-Gly-DJ- | | | | |
| | QWAVGHLM-NH₂* | | | | |
| L64 | DO3A-monoamide-G-Adca3- | 8.5 | 3.3 | — | yes |
| | QWAVGHLM-NH₂* | | | | |
| L63 | DO3A-monoamide-G-Ah12ca- | 23 | 3.8 | — | yes |
| | QWAVGHLM-NH₂* | | | | |
| L67 | DO3A-monoamide-G-Akca- | 5.5 | 2.3 | — | yes |
| | QWAVGHLM-NH₂* | | | | |
| na | DO3A-monoamide-Cha-Cha- | 22 | 77 | yes | — |
| | QWAVGHLM-NH₂* | | | | |
| na | DO3A-monoamide-Nal1-Bip- | 30 | 210.9 | yes | — |
| | QWAVGHLM-NH₂* | | | | |
| na | DO3A-monoamide-Cha-Na11- | 8 | 66.5 | yes | — |
| | QWAVGHLM-NH₂* | | | | |
| na | DO3A-monoamide-Nal1-Bpa4- | 17 | 89.9 | yes | — |
| | QWAVGHLM-NH₂* | | | | |
| L301 | DO3A-monoamide-Amb4- | 10 | 6.8 | | yes |
| | Nal1-QWAVGHLM-NH₂* | | | | |
| L147 | DO3A-monoamide-G- | 4 | 32 | yes | — |
| | Mo3abz4-QWAVGHLM-NH₂* | | | | |
| L241 | DO3A-monoamide-G- | 4 | 0.8 | — | yes |
| | Cl3abz4QWAVGHLM-NH₂* | | | | |
| L242 | DO3A-monoamide-G-M3abz4- | 5 | 2.2 | — | yes |
| | QWAVGHLM-NH₂* | | | | |
| L243 | DO3A-monoamide-G- | 14 | 9.9 | — | yes |
| | Ho3abz4-QWAVGHLM-NH₂* | | | | |
| L202 | DO3A-monoamide-G-Hybz4- | 13 | 2.7 | — | yes |
| | QWAVGHLM-NH₂* | | | | |
| L204 | DO3A-monoamide-Abz4-G- | 50 | 1.2 | — | yes |
| | QWAVGHLM-NH₂* | | | | |
| L233 | DO3A-monoamide-G-Abz3- | 4.8 | 1.6 | — | yes |
| | QWAVGHLM-NH₂* | | | | |
| L235 | DO3A-monoamide-G-Nmabz4- | 7 | 1.5 | — | yes |
| | QWAVGHLM-NH₂* | | | | |
| L147 | DO3A-monoamide-Mo3amb4- | 3.5 | 1.2 | — | yes |
| | QWAVGHLM-NH₂* | | | | |
| L71 | DO3A-monoamide-Amb4- | 7.2 | 0.2 | — | yes |
| | QWAVGHLM-NH₂* | | | | |
| L73 | DO3A-monoamide-Aeb4- | 5 | 1.8 | — | yes |
| | QWAVGHLM-NH₂* | | | | |
| L208 | DO3A-monoamide-Dae-Tpa- | 8 | 0.9 | — | yes |
| | QWAVGHLM-NH₂* | | | | |
| L206 | DO3A-monoamide-G- | 5 | 1.3 | — | yes |
| | A4m2biphc4- | | | | |
| | QWAVGHLM-NH₂* | | | | |
| L207 | DO3A-monoamide-G- | 3 | 15.1 | — | yes |
| | A3biphc3- | | | | |
| | QWAVGHLM-NH₂* | | | | |
| L72 | DO3A-monoamide-Amc4- | 8.2 | 2.6 | — | yes |
| | QWAVGHLM-NH₂* | | | | |
| L107 | DO3A-monoamide-Amc4- | 5 | 0.3 | — | yes |
| | Amc4-QWAVGHLM-NH₂* | | | | |
| L89 | DO3A-monoamide-Aepa4- | 23 | 114 | yes | — |
| | QWAVGHLM-NH₂* | | | | |
| L28 | N,N-dimethylglycine-Ser- | 25 | 13 | — | yes |
| | Cys(Acm)-Gly-Aepa4-S- | | | | |
| | QWAVGHLM-NH₂* | | | | |
| L74 | DO3A-monoamide-G-Inp- | 6.5 | 3.4 | — | Yes |
| | QWAVGHLM-NH₂* | | | | |
| L36 | N,N-dimethylglycine-Ser- | 7 | 12.1 | — | Yes |
| | Cys(Acm)-Gly-Pia1-J- | | | | |
| | QWAVGHLM-NH₂* | | | | |
| L82 | DO3A-monoamide-Ckbp- | 8 | 1.7 | — | Yes |
| | QWAVGHLM-NH₂* | | | | |
| na | DO3A-monoamide-Aoc- | 11 | 14 | — | Yes |
| | QWAVGHL-Nle-NH₂* | | | | |
| L70 | DO3A-monoamide-G-Abz4- | 4.5 | 1.5 | — | Yes |
| | QWAVGHLM-NH₂* | | | | |
| na | DO3A-monoamide- | 366 | >250 | No selective | |
| | QWAVGHLM-NH₂* | | | preference | |
| na | QWAVGHLM-NH₂* | 369 | 754 | No selective | |
| | | | | preference | |
| na | WAVGHLM-NH₂ | >800 | >800 | No selective | |
| | (SEQ ID NO: 25) | | | preference | |
| L204 | DO3A-monoamide-Abz4-G- | >50 | 1.2 | preference | |
| | QWAVGHLM-NH₂* | | | for NMB-R | |
| na | GNLWATGHFM-NH₂ | >500 | 0.7 | preference | |
| | (SEQ ID NO: 24) | | | for NMB-R | |
| L227 | DO3A-monoamide-G-Abz4- | 28 | 0.8 | — | Yes |
| | LWATGHFM-NH₂ wherein | | | | |
| | LWATGHFM-NH₂ is | | | | |
| | SEQ ID NO: 16 | | | | |

*QWAVGHLM-NH₂ is the sequence BBN(7-14) which is SEQ ID NO: 1

In the above Table “na” indicates “not applicable” (e.g. the compound does not contain a linker of the invention and thus was not assigned an L#).

Based on the above, several results were observed. The receptor binding region alone (BBN₇₋₁₄ or BBN₈₋₁₄) did not show any preference to GRP-R or NMB-R. The addition of a chelator alone to the receptor binding region did not contribute to the affinity of the peptide to GRP-R or NMB-R (DO3A-monoamide-QWAVGHLM-NH₂, wherein QWAVGHLM-NH₂ is the BBN(7-14) sequence (SEQ ID NO: 1). Coupling the chelator to the peptide through a linker did contribute to the affinity of the peptide towards the receptor. However, depending on the type of linker this affinity varied from being dual (preference for both NMB-R and GRP-R) to GRP-R (preferring GRP-R). In the above paragraph, QWAVGHLM-NH₂ is the BBN(7-14) sequence (SEQ ID NO: 1).

The ω-Aminoalkanoic acids tested (8-Aminooctanoic acid in ¹⁷⁵Lu-DO3A-monoamide-Aoc-QWAVGHLM-NH₂ and DO3A-monoamide-Aoc-QWAVGHL-Nle-NH₂, 3-aminopropionic acid in DO3A-monoamide-Apa3-QWAVGHLM-NH₂ and 4-aminobutanoic acid in DO3A-monoamide-Abu-4-QWAVGHLM-NH₂) as linkers, conferred the peptide with dual affinity for both GRP-R and NMB-R. Replacement of ‘Met’ in ¹⁷⁵Lu-DOTA-Aoc-QWAVGHLM-NH₂ by ‘Nle’ did not change this dual affinity of the peptide. In the above paragraph, QWAVGHLM-NH₂ is the BBN(7-14) sequence (SEQ ID NO: 1).

Cholic acid containing linkers (3-aminocholic acid in L64, 3-amino-12-hydroxycholanic in L63 and 3-amino-12-ketocholanic in L67 conferred the peptides with dual affinity for both GRP-R and NMB-R. Cycloalkyl and aromatic substituted alanine containing linkers (3-cyclohexylalanine in DO3A-monoamide-Cha-Cha-QWAVGHLM-NH₂, 1-Naphthylalanine in DO3A-monoamide-Cha-Nal1-QWAVGHLM-NH₂, 4-Benzoylphenylalanine in DO3A-monoamide-Nal1-Bpa-4-QWAVGHLM-NH₂ and Biphenylalanine in DO3A-monoamide-Nal1-Bip-QWAVGHLM-NH₂) imparted the peptides with selective affinity towards GRP-R. A linker containing only 4-(2-Aminoethylpiperazine)-1 also contributed to the peptides with GRP-R selectivity (L89). In the above paragraph, QWAVGHLM-NH₂ is the BBN(7-14) sequence (SEQ ID NO: 1).

Introduction of a G-4-amino benzoic acid linker to the NMB sequence conferred the compound with an affinity to GRP-R in addition to its inherent NMB-R affinity (L227 vs GNLWATGHFM-NH₂ (SEQ ID NO: 24). Shifting the position of Gly around the linker altered the affinity of L70 from its dual affinity to a selective affinity to NMB-R (L204). 3-methoxy substitution in 4-aminobenzoic acid in L70 (as in L240) changed the dual affinity to a selective affinity to GRP-R.

It is apparent from the preceding data that the linker has a significant effect on the receptor subtype specificity. Three groups of compounds can be identified:

Those that are Active at the GRP-R

These compounds provide information specific to this receptor in vitro and in vivo, which can be used for diagnostic purposes. When these compounds are radiolabeled with a therapeutic radionuclide, therapy can be performed on tissues containing only this receptor, sparing those that contain the NMB-R

Those that are Active at the NMB-R

These compounds provide information specific to this receptor in vitro and in vivo, which can be used for diagnostic purposes. When radiolabeled with a therapeutic radionuclide, therapy can be performed on tissues containing only this receptor, sparing those that contain the GRP-R

Those that are Active at Both the GRP-R and the NMB-R

These compounds provide information on the combined presence of these two receptor subtypes in vitro and in vivo, that can be used for diagnostic purposes. Targeting both receptors may increase the sensitivity of the examination at the expense of specificity. When these compounds are radiolabeled with a therapeutic radionuclide, therapy can be performed on tissues containing both receptors, which may increase the dose delivered to the desired tissues.

### Example LXI

### Competition Studies of Modified Bombesin (BBN) analogs with ¹²⁵I-BBN for GRP-R in Human Prostate Cancer (PC3) Cells

To determine the effect of replacing certain amino acids in the BBN⁷⁻¹⁴ analogs, peptides modified in the targeting portion were made and assayed for competitive binding to GRP-R in human prostate cancer (PC3) cells. All these peptides have a common linker conjugated to a metal chelating moiety (DOTA-Gly-Abz4-). The binding data (IC₅₀ nM) are given below in Table 15.

A. Materials and Methods:

1. Cell Culture:

PC3 cell lines were obtained from ATCC(CRL-1435) and cultured in RPMI 1640 (ATCC) supplemented with 2 mM L-glutamine, 1.5 g/L Sodium bicarbonate, 10 mM HEPES and 10% FBS. Cultures were maintained in a humidified atmosphere containing 5% CO₂/95% air at 37° C. PC3 cells for the assays were plated at a concentration of 2.0×10⁴ cells/well in a 96-well white/clear bottom plates (Falcon Optilux-I). Plates were used for the assays on day 2 of the post-plating.

2. Binding Buffer:

RPMI 1640 (ATCC) containing 25 mM HEPES, 0.2% BSA fraction V, 1.0 mM AEBSF (CAS #3087-99-7) and 0.1% Bacitracin (CAS #1405-87-4), pH 7.4.

3. ¹²⁵I-Tyr⁴-Bombesin [¹²⁵I-BBN]

¹²⁵I-BBN (Cat #NEX258) was obtained from PerkinElmer Life Sciences.

C. In Vitro Assay:

Competition assay with ¹²⁵I-BBN for GRP-R in PC3 cells:

All compounds tested were dissolved in binding buffer and appropriate dilutions were also done in binding buffer. PC3 cells for assay were seeded at a concentration of 2.0×10⁴/well either in 96-well black/clear collagen I cellware plates (Beckton Dickinson Biocoat). Plates were used for binding studies on day 2 post-plating. The plates were checked for confluency (>90% confluent) prior to assay. For competition assay, N,N-dimethylglycyl-Ser-Cys(Acm)-Gly-Ava5-QWAVGHLM-NH₂, wherein QWAVGHLM-NH₂ is the BBN(7-14) sequence (SEQ ID NO: 1) (control) or other competitors at concentrations ranging from 1.25×10⁻⁹ M to 500×10⁻⁹ M, was co-incubated with ¹²⁵I-BBN (25,000 cpm/well). The studies were conducted with an assay volume of 75 L per well. Triplicate wells were used for each data point. After the addition of the appropriate solutions, plates were incubated for 1 hour at 4° C. Incubation was ended by the addition of 200 μL of ice-cold incubation buffer. Plates were washed 5 times and blotted dry. Radioactivity was detected using either a LKB CompuGamma counter or a microplate scintillation counter. The bound radioactivity of ¹²⁵I-BBN was plotted against the inhibition concentrations of the competitors, and the concentration at which ¹²⁵I-BBN binding was inhibited by 50% (IC₅₀) was obtained from the binding curve.

TABLE 15
Competition studies with ¹²⁵I-BBN for GRP-R in PC3 cells

| | L # | PEPTIDES | IC₅₀ [nM] |
|---|---|---|---|
| Ref | Na | N,N-dimethylglycyl-Ser-Cys(Acm)-Gly-Ava5- | 2.5 |
| | | QWAVGHLM-NH₂* | |
| 1 | L70 | DO3A-monoamide-G-Abz4-QWAVGHLM-NH₂* | 4.5 |
| 2 | L214 | DO3A-monoamide-G-Abz4-fQWAVGHLM-NH₂* | 18 |
| 3 | L215 | DO3A-monoamide-G-Abz4-QRLGNQWAVGHLM-NH₂ | 6 |
| | | (wherein QRLGNQWAVGHLM-NH₂ is SEQ ID NO: 3) | |
| 4 | L216 | DO3A-monoamide-G-Abz4-QRYGNQWAVGHLM-NH₂ | 4.5 |
| | | (wherein QRYGNQWAVGHLM-NH2 is SEQ ID NO: 4) | |
| 5 | L217 | DO3A-monoamide-G-Abz4-QKYGNQWAVGHLM-NH₂ | 10 |
| | | (wherein QKYGNQWAVGHLM-NH₂ is SEQ ID NO: 5) | |
| 6 | L218 | >EQ-[K(DO3A-monoamide-G-Abz4)-LGNQWAVGHLM- | 53 |
| | | NH₂ (wherein LGNQWAVGHLM-NH₂ is SEQ ID NO: 18) | |
| 7 | L219 | DO3A-monoamide-G-Abz4-fQWAVGHLM-NH-C₅H₁₂ | 75 |
| | | (wherein QWAVGHLM-NH-C₅H₁₂ is SEQ ID NO: 21) | |
| 8 | L220 | DO3A-monoamide-G-Abz4-QWAVaHLM-NH₂ | 13 |
| | | (wherein QWAVaHLM-NH₂ is SEQ ID NO: 14) | |
| 9 | L221 | DO3A-monoamide-G-Abz4-fQWAVGHLL-NH₂ | 340 |
| | | (wherein QWAVGHLL-NH₂ is SEQ ID NO: 8) | |
| 10 | L222 | DO3A-monoamide-G-Abz4-yQWAV-Ala2-HF-Nle-NH₂ | 46 |
| | | (wherein QWAV-Ala2-HF-Nle-NH₂ is SEQ ID NO: 23) | |
| 11 | L223 | DO3A-monoamide-G-Abz4-fQWAV-Ala2-HF-Nle-NH₂ | 52 |
| | | (wherein QWAV-Ala2-HF-Nle-NH₂ is SEQ ID NO: 23) | |
| 12 | L224 | DO3A-monoamide-G-Abz4-QWAGHFL-NH₂ | >500 |

| | | | |
|---|---|---|---|
| | | (wherein QWAGHFL-NH₂ is SEQ ID NO: 10) | |
| 13 | L225 | DO3A-monoamide-G-Abz4-LWAVGSFM-NH₂ | 240 |
| | | (wherein LWAVGSFM-NH₂ is SEQ ID NO: 11) | |
| 14 | L226 | DO3A-monoamide-G-Abz4-HWAVGHLM-NH₂ | 5.5 |
| | | (wherein HWAVGHLM-NH₂ is SEQ ID NO: 12) | |
| 15 | L227 | DO3A-monoamide-G-Abz4-LWATGHFM-NH₂ | 39 |
| | | (wherein LWATGHFM-NH₂ is SEQ ID NO: 16) | |
| 16 | L228 | DO3A-monoamide-G-Abz4-QWAVGHFM-NH₂ | 5.5 |
| | | (wherein QWAVGHFM-NH₂ is SEQ ID NO: 13) | |
| 17 | na | GNLWATGHFM-NH₂ (SEQ ID NO: 24) | >500 |
| 18 | na | yGNLWATGHFM-NH₂ | 450 |
| | | (wherein GNLWATGHFM-NH₂ is SEQ ID NO: 24) | |
| 19 | L300 | DO3A-monoamide-G-Abz4-QWAVGHFL-NH₂ | 2.5 |
| | | (wherein QWAVGHFL-NH₂ is SEQ ID NO: 22) | |

*QWAVGHLM-NH₂ is the BBN(7-14) sequence (SEQ ID NO: 1)

Results/Conclusions: Analysis of the binding results of various peptides modified in the targeting portion indicated the following:

Neuromedin analogs (GNLWATGHFM-NH₂, yGNLWATGHFM-NH₂ wherein GNLWATGHFM-NH₂ is SEQ ID NO: 24) are unable to compete for the GRP-R except when conjugated to DO3A-monoamide-G-Abz4 (L227). They are, however, effective NMB competitors. This is similar to the requirement for derivatization of the amino end of the bombesin sequence as reflected in QWAVGHLM-NH₂, DO3A-monoamide-QWAVGHLM-NH₂ (wherein QWAVGHLM-NH₂ is the BBN(7-14) sequence SEQ ID NO: 1) and L70. Replacement of the histidine (L225) reduces competition at the GRP-R.

Reversal of the two linker components in L70 to give L204 changes the subtype specificity to favor the NMB subtype. L¹³F substitution in the bombesin sequence maintains GRP-R activity. (L228).

TABLE 16

| | | IC₅₀ | |
|---|---|---|---|
| L Number | Sequence | C6/NMB-R | PC3/GRP-R |
| na | GNLWATGHFM-NH₂ | 0.69 | >500 |
| | (SEQ ID NO: 24) | | |
| na | yGNLWATGHFM-NH₂ | 0.16 | 884.6 |
| | (wherein GNLWATGHFM-NH₂ is SEQ ID NO: 24) | | |
| L227 | DO3A-monoamide-G-Abz4-LWATGHFM-NH₂ | 0.07 | 28.0 |
| | (wherein LWATGHFM-NH₂ is SEQ ID NO: 16) | | |
| L225 | DO3A-monoamide-G-Abz4-LWAVGSFM-NH₂ | — | 240 |
| | (wherein LWAVGSFM-NH₂ is SEQ ID NO: 11) | | |
| na | WAVGHLM-NH₂ (SEQ ID NO: 25) | >800 | >800 |
| na | QWAVGHLM-NH₂* | 369 | 754 |
| na | DO3A-monoamide-QWAVGHLM-NH₂* | 161 | 366 |
| L70 | DO3A-monoamide-G-Abz4-QWAVGHLM-NH₂* | 4.5 | 1.5 |
| L204 | DO3A-monoamide-Abz4-GQWAVGHLM-NH₂ | 1.19 | >50 |
| | (wherein GQWAVGHLM-NH₂ is SEQ ID NO: 19) | | |
| L228 | DO3A-monoamide-G-Abz4-QWAVGHFM-NH₂ | — | 5.5 |
| | wherein QWAVGHFM-NH₂ is SEQ ID NO: 13) | | |

*QWAVGHLM-NH₂ is the BBN(7-14) sequence (SEQ ID NO: 1)

As seen here, F¹³M¹⁴ to F¹³L¹⁴ substitution in L228 produces a compound (L300) with high activity at the GRP-R. The removal of the methionine has advantages as it is prone to oxidation. This benefit does not occur if the L¹³F substitution is not also performed. (L221) Removal of V¹⁰ resulted in complete loss of binding as seen in L224.

TABLE 17

| | | IC50 | |
|---|---|---|---|
| Number | Sequence | C6/NMB-R | PC3/GRP-R |
| L300 | DO3A-monoamide-G-Abz4-QWAVGHFL-NH₂ | — | 2.5 |
| | (wherein QWAVGHFL-NH₂ is SEQ ID NO: 22) | | |
| L221 | DO3A-monoamide-G-Abz4-fQWAVGHLL-NH₂ | — | 340 |
| | (wherein QWAVGHLL-NH₂ is SEQ ID NO: 8) | | |
| L224 | DO3A-monoamide-G-Abz4-QWAGHFL-NH₂ | — | >500 |
| | (wherein QWAGHFL-NH₂ is SEQ ID NO: 10) | | |

TABLE 18
As seen in Table 18,various substitutions are allowed in
the BBN²⁻⁶ region (L214-L217, L226)

| | | IC50 | |
|---|---|---|---|
| Number | Sequence | C6/NMB-R | PC3/GRP-R |
| na | pEQRYGNQWAVGHLM-NH₂ (SEQ ID NO: 28) | 3.36 | 2.2 |
| L214 | DO3A-monoamide-G-Abz4-fQWAVGHLM-NH₂* | — | 18 |
| L215 | DO3A-monoamide-G-Abz4- | — | 6 |
| | QRLGNQWAVGHLM-NH₂ (wherein | | |
| | QRLGNQWAVGHLM-NH₂ is SEQ ID NO: 3) | | |
| L216 | DO3A-monoamide-G-Abz4- | — | 4.5 |
| | QRYGNQWAVGHLM-NH₂ (wherein | | |
| | QRYGNQWAVGHLM-NH₂ is SEQ ID NO: 4) | | |
| L217 | DO3A-monoamide-G-Abz4- | — | 10 |
| | QKYGNQWAVGHLM-NH₂ (wherein | | |
| | QKYGNQWAVGHLM-NH₂ is SEQ ID NO: 5) | | |
| L226 | DO3A-monoamide-G-Abz4-HWAVGHLM-NH₂ | — | 5.5 |
| | (wherein HWAVGHLM-NH₂ is SEQ ID NO: 12) | | |

*QWAVGHLM-NH₂ is the BBN(7-14) sequence (SEQ ID NO: 1)

TABLE 19
As expected,results from Table 19 show that the universal
agonists (L222 and L223) compete reasonably well at ~50 nM level.

| | | | IC₅₀ | |
|---|---|---|---|---|
| Name | Number | Sequence | C6/NMB-R | PC3/GRP-R |
| Universal | L222 | DO3A-monoamide-G-Abz4- | — | 46 |
| agonist | | yQWAV-Ala2-HF-Nle-NH₂ | | |
| | | (wherein QWAV-Ala2-HF- | | |
| | | Nle-NH₂ is SEQ ID NO: 23) | | |
| Universal | L224 | DO3A-monoamide-G-Abz4- | — | 52 |
| agonist | | fQWAV-Ala2-HF-Nle-NH₂ | | |
| | | (wherein QWAV-Ala2-HF- | | |
| | | Nle-NH₂ is SEQ ID NO: 23) | | |

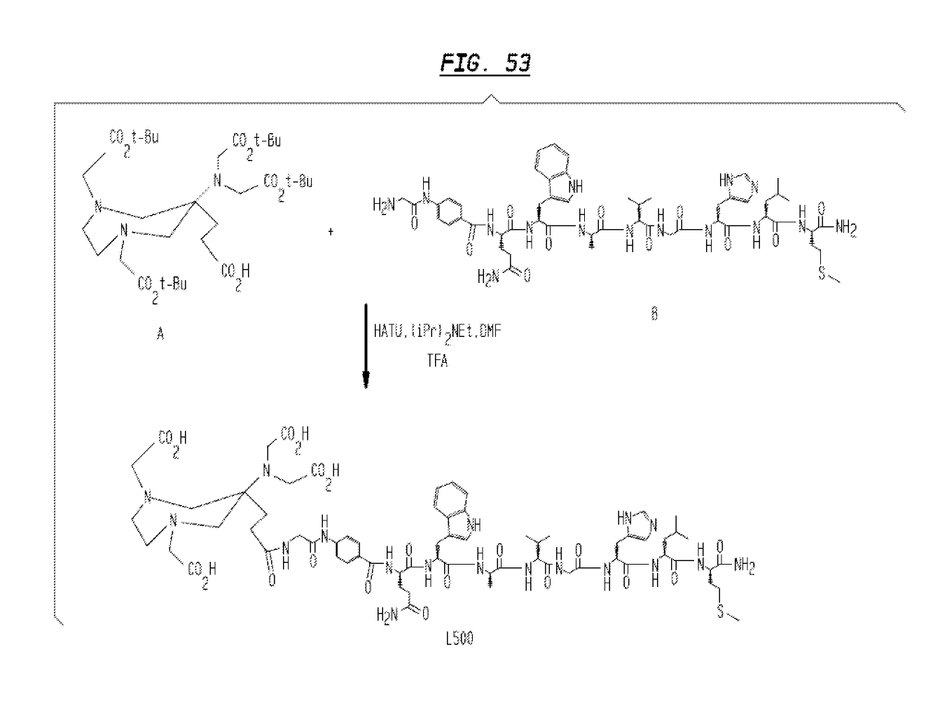

The compound L500 was prepared as illustrated in FIG. 53. Specifically, diisopropylethylamine (150 μL) was added to a cooled solution of the acid A (0.19 g, 0.3 mmol) and HATU (0.12 g, 0.32 mmol) in DMF (1 mL) and stirred for 5 min. Purified peptide B (0.11 g, 0.1 mmol) was then added to the reaction mixture and stirred for 18 h. DMF was removed and the oil obtained was dissolved in a mixture of DMF/CH₃CN and purified by preparative HPLC. Pure fractions containing the tetra-t-butyl ester were collected and freeze dried to give the tetra-t-butyl ester as a white solid. Yield 80 mg (32%). Tetra-t-butyl ester obtained was dissolved in reagent B and stirred for 8 h. TFA was removed and the resulting pasty solid was purified by HPLC using CH₃CN/Water/0.1% TFA. Pure fractions were collected and freeze dried to give L500 as a white solid. Yield 23 mg (38%) MS: 1515.7 (M−H), 757.4 (M−2H)/2.

### Example LXIII

### Synthesis of L501

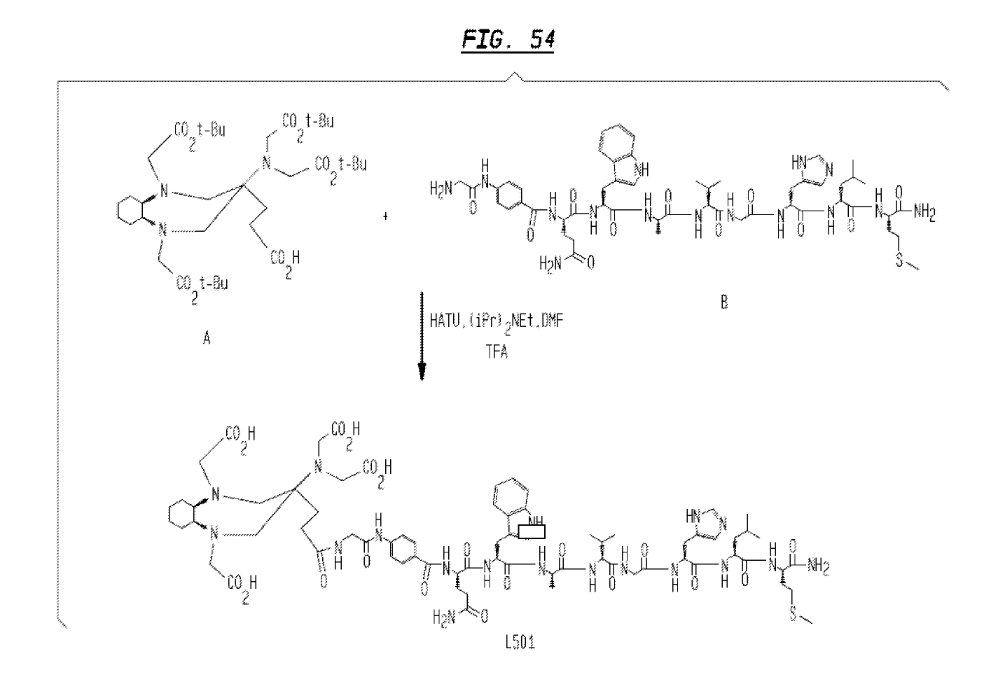

### FIG. 54

The compound L501 was prepared as illustrated in FIG. 54. Diisopropylethylamine (150 μL) was added to a cooled mixture of A (0.278 g, 0.4 mmol) and HATU (0.152 g, 0.4 mmol) in DMF (1 mL) and stirred for 5 min. Purified peptide B (0.12 g 0.11 mmol) was added to the reaction mixture and stirred for 18 h. DMF was removed and the oil obtained was dissolved in a mixture of DMF/CH₃CN and purified by preparative HPLC. Pure fractions containing the tetra-t-butyl ester were collected and freeze dried to give the tetra t-butylester. Yield 62 mg (32%). Tetra t-butyl ester (36.0 mg, 0.02 mmol) was dissolved in reagent B and stirred for 8 h. TFA was removed and the resulting thick oil was purified by HPLC using CH₃CN/Water/0.1% TFA. Pure fractions were collected and freeze dried to give L501 as a white solid. Yield 12 mg (38%). MS: 1569.7 (M−H), 784.4 (M−2H/2), 803.3 (M+K−2H)/2.

### Example LXIV

### Radiolabeling (¹⁷⁷Lu) and Biodistribution of L500 and L501 Radiolabeling and HPLC Analysis of ¹⁷⁷Lu-Complexes of L500 and L501

### Radiolabeling Procedure:

Typically, a 1 mg/mL solution of ligand was prepared in 0.2 M sodium acetate buffer (pH 4.8). An aliquot of this solution (2 to 5 μL) and 6 to 10 mCi of ¹⁷⁷LuCl₃ (in 0.05 N HCl, specific activity 2.8-4.09 Ci/μmol) were added to 100 to 200 μL of 0.2 M, pH 4.8 NaOAc buffer to achieve a ligand to Lu molar ratio of 2:1. After incubation at room temperature for 5 min, 10 μL of 10 mM Na₂EDTA.2H₂O was added to terminate the reaction and scavenge any remaining free ¹⁷⁷Lu in the solution. A 9:1 (v/v) mixture of Bacteriostatic 0.9% Sodium Chloride Injection USP/ASCOR L500® Ascorbic Acid Injection USP (0.2 mL) was then added to inhibit radiolysis of the resulting radiocomplex. The radiochemical purity (RCP) was determined by HPLC. Complete coordination of Lu-177 was observed within 5 min of incubation at room temperature for all the tested ligands.

### Radiolabeled Complex Prepared for In Vivo Biodistribution Studies:

For biodistribution studies, the radiolabeled compounds were prepared as described above except that a 1:1 molar ratio of ligand to Lutetium was used to guarantee complete chelation of all starting ligand. The HPLC peak containing the resulting ¹⁷⁷Lu complex was collected in 1 mL of 9:1 Bacteriostatic saline/ASCOR L500® solution containing 0.1% HSA, and the organic solvents were removed using a speed-vacuum device. The remaining solution was further diluted to the required radioconcentration using Bacteriostatic saline/ASCOR L500® Ascorbic Acid Injection USP mixed in a 9 to 1 [v/v]) ratio. The radiochemical purity of all samples was ≧95%.

HPLC analysis: All HPLC studies were performed at a flow rate of 1.5 mL/min using a column temperature of 37° C.

### 1. 177Lu-L500

HPLC column: Zorbax Bonus-RP, 5 μm, 80 Å pore size, 250 mm×4.6 mm (Agilent).
Mobile phase: The following gradient was used, where A=water; B=water containing 30 mM (NH₄)₂SO₄; C=methanol; D=acetonitrile

TABLE 20

| Time | A (%) | B (%) | C (%) | D (%) |
|---|---|---|---|---|
| 0-2 min | 70 | 30 | 0 | 0 |
| 15 min | 36 | 30 | 16 | 16 |
| 30 min | 30 | 30 | 20 | 20 |
| 35-40 min | 0 | 30 | 35 | 35 |
| 45 min | 70 | 30 | 0 | 0 |
| 55 min | 70 | 30 | 0 | 0 |

Retention time: ¹⁷⁷Lu-L500=25.5 min.

### 2. 177Lu-L501

HPLC column: Zorbax Bonus-RP, 5 μm, 80 Å pore, 250 mm×4.6 mm (Agilent).
Mobile phase: The following gradient was used, where A=water; B=water containing 30 mM (NH₄)₂SO₄; C=methanol; D=acetonitrile.

TABLE 21

| Time | A (%) | B (%) | C (%) | D (%) |
|---|---|---|---|---|
| 0-2 min | 70 | 30 | 0 | 0 |
| 15 min | 32 | 30 | 19 | 19 |
| 30 min | 28 | 30 | 21 | 21 |
| 35-40 min | 0 | 30 | 35 | 35 |
| 45 min | 70 | 30 | 0 | 0 |
| 55 min | 70 | 30 | 0 | 0 |

Retention time: ¹⁷⁷Lu-L501=23.1 min.

### Biodistribution Studies:

The tumor targeting capacity, biodistribution and kinetics of ¹⁷⁷Lu-L500, ¹⁷⁷Lu-XX100, ¹⁷⁷Lu-L501 and ¹⁷⁷Lu-L70 were evaluated in the human PC-3 nude mouse model. 10-50 μCi of the HPLC purified compounds were administered to each mouse by i.v. tail vein injection, n=4 per group. At 1 h, 1 and 7 days post injection, the mice were terminated and the organs and tissues were harvested. Radioactivity was assayed in a gamma counter. The data was expressed as percentage of the total administered radioactivity (% ID) for the urine combined with the bladder, as well as for the blood pool; and percentage of the total administered radioactivity per gram (% ID/g) for all the other tested organs.

TABLE 22

| | Tumor | Tumor | Tumor | Blood | Blood | Blood | Urine/ | Femur | Carcass |
|---|---|---|---|---|---|---|---|---|---|
| | 1 h % | 24 h % | 7 d | 1 h | 24 h | 7 d | Blad 1 h | 7 d | 7 d |
| Cmpd | ID/g | ID/g | % ID/g | % ID | % ID | % ID | % ID | % ID/g | % ID/g |
| XX100 | 0.47 ± .21 | 0.03 ± 0.01 | 0.01 ± 0.00* | 0.39 ± 0.29 | 0.01 ± 0.01 | 0.00 ± 0.00* | 53.9 ± 30.6 | 0.19 ± 0.04* | 0.38 ± 0.02* |
| L500 | 4.49 ± 1.72 | 1.89 ± 0.55 | 0.49 ± 0.13 | 0.39 ± 0.07 | 0.01 ± 0.00 | 0.00 ± 0.00 | 56.6 ± 12.3 | 0.14 ± 0.02 | 0.50 ± 0.01 |
| L501 | 1.82 ± 0.06 | 0.76 ± 0.35 | 0.12 ± 0.04 | 1.82 ± 0.06 | 0.00 ± 0.00 | 0.00 ± 0.00 | 51.9 ± 19.7 | ND | 0.07 ± 0.1 |
| L70 | 5.86 ± 1.91 | 1.82 ± 0.06 | 0.34 ± 0.16 | 1.23 ± 0.58 | 0.02 ± 0.00 | 0.00 ± 0.00 | 43.4 ± 4.3 | 0.06 ± 0.03 | 0.17 ± 0.01 |

*72 h timepoint,
ND—not done

The data show that the Lu-177 administered as a complex of the underivatized cyclohexylaazta chelator (XX100), which does not include a GRP receptor targeting moiety, is rapidly cleared from the body with little residual localization in any organs or tissue. When the complex (or its closely related derivative) is derivatized with the GRP targeting peptide (as in L500 and L501) the radioactivity shows localization in the tumor. The data are similar to those of ¹⁷⁷Lu-L70, which as shown herein, has demonstrated efficacy for delivering radioactivity to PC-3 tumors for radiotherapeutic purposes. The tumor localization and lack of retention of radioactivity in the other tissue of the body show the utility of compounds of the invention containing these two chelators for radioimaging and radiotherapy.

The structure of ¹⁷⁷Lu-XX100 is:

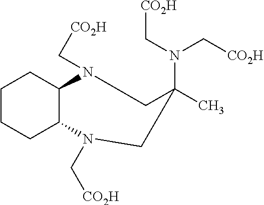

### Example LXV

### Reduction of Aberrant Vascular Permeability in LNCaP Tumors

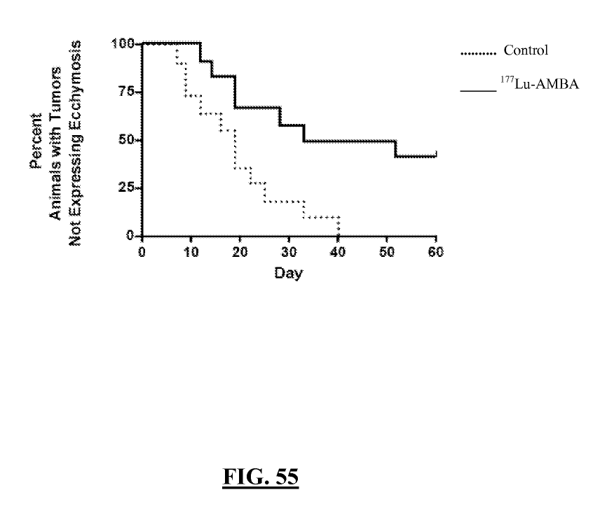

### FIGS. 55-57

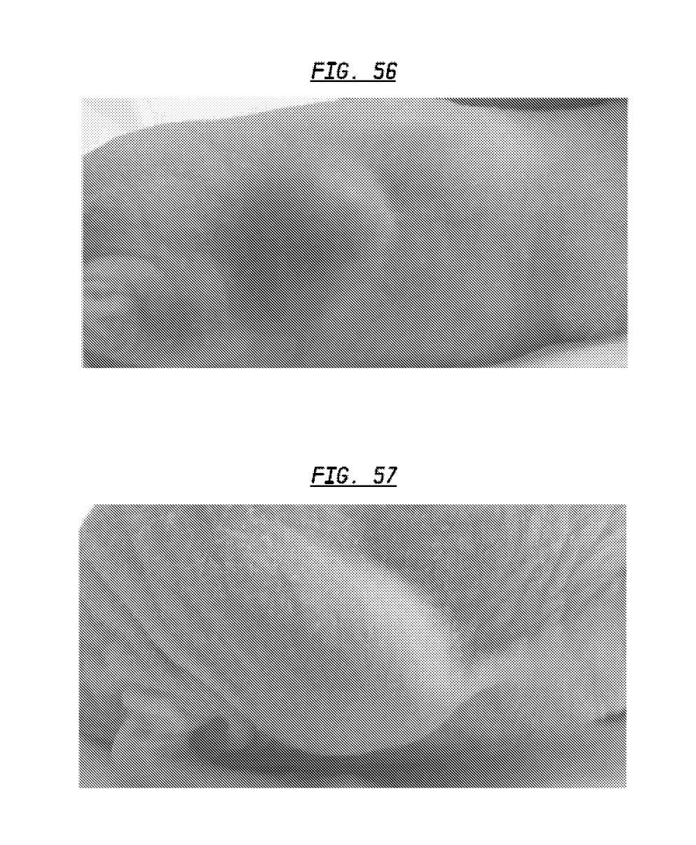

Referring now to FIG. 55, in a preferred embodiment, LNCaP cells grown as xenografts in CrTAC:NCr:Foxn1^{nu/nu} mice exhibit a low profile invasive habit with extensive extravasation of blood from the tumor vasculature into the skin, resulting in a nonelevated, rounded or irregular, dark or darker patch (ecchymosis) (FIG. 56). Ecchymosis is clearly visible and provides a measure of the leakiness of the tumour vasculature. It has now been shown that treatment of LNCaP tumours with ¹⁷⁷Lu-L70 decreases ecchymosis, indicating that it decreases aberrant vascular permeability, as is shown in FIGS. 55 and 57. Due to its effects on vascular permeability, treatment with radioactive L70 in combination with another therapeutic agent would be expected to improve the delivery of the other therapeutic agent.

As shown in FIGS. 55-57, in a preferred embodiment, radiotherapy studies were performed using the LNCaP (androgen sensitive prostate adenocarcinoma) tumor-bearing nude mouse model. The ¹⁷⁷Lu labeled compound of the invention was compared to an untreated control group. (n=12 for each group for 60 days), Treated mice were administered 100 μL of ¹⁷⁷Lu-labeled compound of the invention at 30 mCi/kg total dose, i.v., or s.c. under sterile conditions. The subjects were housed in a barrier environment for the duration of the study. Body weight, tumor size (by caliper measurement), and clinical observations were collected on each subject 3 times per week for the duration of the study. Criteria for early termination included: death; loss of total body weight (TBW) equal to or greater than 20%; tumor size equal to or greater than 2 cm³. Treatment with ¹⁷⁷Lu-L70 does not increase survival over the control animals given no treatment, but the mice treated with ¹⁷⁷Lu-L70 had a significant reduction in observable ecchymosis, P=0.0056. The occurrence of ecchymosis over the duration of the study is shown in FIG. 55, and is depicted in FIGS. 56 and 57.

Time to Progression is an alternative means to assess the anti-cancer activity of new agents. It is defined as the time point at which the tumor shows a 20% increase in diameter. The Mean Time to Progression is the study day when half of the animals in a group reach that point. In a preferred embodiment, it has now been found that the mean time to tumor progression in LNCap tumours is increased by about 100% with ¹⁷⁷Lu-L70 treatment. The Mean Time to Progression data is shown in Table 23.

TABLE 23

| | Mean Time to Progression |
|---|---|
| Group | (Study Day) |
| Control | 14 |
| ¹⁷⁷Lu-L70 | 28 |

Time to Progression=increase of at least 20%>2r, where r=average (L×W)/2.

As shown in FIG. 56, control mice which did not receive ¹⁷⁷Lu-L70 had LNCaP xenografts which presented with ecchymosis extending into the ipsilateral hind limb. By comparison, in FIG. 57, experimental mice which received ¹⁷⁷Lu-L70 had LNCaP xenografts showing reduced ecchymosis when compared to control mice.

As described in this example, treatment with ¹⁷⁷Lu-L70 provides a beneficial response in the LNCap tumours, that is, normalization of blood vessels in the tumour and substantially increased time to progression.

### Example LXVI

### In Vitro Demonstration of the Effect of Lapatinib on GRP Receptor Binding in BT-474 Human Breast Cancer Cells

BT-474 breast cancer cells (human primary invasive ductal carcinoma; ER+, PR+, amplified HER2/neu+) were obtained from the American Type Culture Collection. The growth medium for BT-474 cells is Hybri-care Medium (Cat No: 46X, ATCC) supplemented with 10% heat inactivated FBS (Hyclone, SH30070.03), 0.2% NaHCO₃, and antibiotic/antimycotic “PSF-1x” (GibcoBRL, 15240-062) for a final concentration of penicillin-streptomycin (1×, 100 units/mL), and fungizone (0.25 g/mL). BT-474 were maintained on CellBind™ Tissue culture flasks (Corning), in a humidified atmosphere containing 5% CO₂/95% air at 37° C., and passaged routinely using 0.05% trypsin/EDTA (GibcoBRL 25300-054). Cells for binding experiments were plated on Day 0 at 5.6×10⁴ cells/cm² in 96-well white/clear bottom microtiter plates (Falcon Optilux-I), or 96 well black/clear collagen I cellware plates (Beckton Dickinson Biocoat).

### Binding Buffer:

RPMI 1640 supplemented with 20 mM HEPES, 0.1% BSA (w/v), 0.5 mM PMSF (AEBSF), bacitracin (50 mg/500 ml), pH 7.4.

### Lapatinib Solutions:

Stock: 1 mM stock in 100% DMSO
Working: 0.1-1.0 M in complete growth medium

### Cell Treatment and Binding Assay:

Treatment: BT-474 cells were plated on Day 0 (see above); approximately 24-48 h after plating, treatment Days 1 and 2 (D_{T}1, D_{T}2), the medium was removed and replaced with fresh growth medium (control), or growth medium with the addition of lapatinib (0.1-1.0 μM).
Cell Binding Assay: On day 3 after washing twice with binding buffer to remove serum proteins, the cells were incubated with ¹⁷⁷Lu-AMBA (serial dilution 5 μCi/mL-0.625 μCi/mL) for 60 min, at 22° C. Incubations were stopped by the addition of 200 μL of ice-cold binding buffer. Plates were washed three more times with binding buffer, 140 μL of scintillation fluid was added to each well, the plates were sealed and analyzed on a Microp plate reader (Microbeta Trilux, Perkin-Elmer).

Treatment with lapatinib resulted in an increase in GRP receptor specific signal per cell in BT-474 breast cancer cells as indicated by an increase in ¹⁷⁷Lu-AMBA (also called ¹⁷⁷Lu-L70) binding of up to 140% of untreated control (0.1-1.0 μM). In addition, up to a 65% decrease in cell proliferation was seen at the highest concentration, possibly indicating an amplification of GRP receptor specific signal. The aforementioned demonstrates cross-talk between EGFR and/or HER2 and GRP-R. The increase could be interpreted as either an alternative pathway to evade destruction (i.e. a switch to GRP for continued growth, or transactivation of EGFR by GRPR), which could be exploited therapeutically as an early indication of resistance to the original therapeutic drug. The increase in signal could also be interpreted as a last attempt to evade tumor cell death (“last gasp”), ultimately leading to the death of the tumor. The ability to detect such changes by imaging would be a tremendous advantage.

Results for this Example through Example XCII are recorded in Table 24 (below), which shows that drug treatment not targeted at the GRP receptor modulates GRP-R uptake of ¹⁷⁷Lu-AMBA (and by inference ^{67/68}Ga-AMBA) on a viable cell basis in vitro in cell cultures:

TABLE 24

| | Prostate Cancer | Breast cancer | | Concentration |
|---|---|---|---|---|
| Drug | PC-3 | T47D | BT-474 | Range (μM) |
| Lapatinib | NC | 50% ↑ | 90-140% ↑ | 0.1-1.0 |
| Dasatinib | 250% ↑ | 500% ↑ | 10-15% ↓ | 0.1-1.0 |
| Gefitinib | 20-40% ↓ | 20% ↑ | NC | 0.1-10 |
| Imatinib | 15-25% ↓ | 15% ↑ | NC | 0.1-1.0 |
| Erlotinib | 5-20% ↓ | 5-20% ↑ | NC | 0.1-1.0 |
| Sorafenib | 10-20% ↓ | NC | 10-25% ↓ | 0.1-1.0 |
| Sunitinib | 10% ↑ | 13% ↑ | 8% ↓ | 0.1-1.0 |
| Anastrozole | 10% ↓ | 10% ↓ | NC | 0.1-1.0 |
| Bortezomib | 60% ↓ | 50% ↓ | 50% ↓ | 0.001-0.1 |

X = Tested (N = 1);
NC: Little (<10%) or no change in Lu-AMBA uptake

The above table demonstrates the in vitro crosstalk between the receptors targeted by the drugs on the left and the GRP receptor in prostate and breast cancer cell lines as detected by a receptor-ligand binding assay.

### Example LXVII

### In Vitro Demonstration of the Effect of Lapatinib on GRP Receptor Binding in T47D Human Breast Cancer Cells

T-47D breast cancer cells (human metastatic mammary gland ductal adenocarcinoma isolated from pleural effusion, ER+, PR+) were obtained from the American Type Culture Collection. The growth medium for T-47D is RPMI 1640 (10-041-CM, Mediatech, Inc), supplemented with 10% heat inactivated FBS (Hyclone, SH30070.03), 0.5% insulin (10516-5mL, Sigma), and PSF-1× and the cells were maintained as in Example LXVI. The binding buffer, lapatinib solutions, treatment method and cell binding assay is the same as in Example LXVI.

Treatment with lapatinib resulted in an increase in GRP receptor specific signal per cell in T-47D breast cancer cells as indicated by an increase in 17Lu-AMBA binding of up to 50% of untreated control (0.1-1.0 μM), with up to a 35% decrease in cell proliferation at the highest concentration. The aforementioned demonstrates cross-talk between EGFR and/or HER2 and GRP-R; interpretation identical to Example LXVI. Results are recorded in Table 24 in Example LXVI.

### Example LXVIII

### In Vitro Demonstration of the Effect of Lapatinib on GRP Receptor Binding in PC-3 Prostate Cancer Cells

PC-3 (human prostate adenocarcinoma, bone metastasis, AR-, ER) were obtained from the American Type Culture Collection. The growth medium for PC-3 cells is RPMI 1640 (10-041-CM, Mediatech, Inc), supplemented with 10% heat inactivated FBS (Hyclone, SH30070.03) and PSF-1x (as in Example LXVI). The binding buffer, lapatinib solutions, treatment method and cell binding assay is the same as in Example LXVI.

Results: Treatment with lapatinib resulted in no change in proliferation or GRP receptor specific signal per cell in PC-3 prostate cancer cells as indicated by equivalent ¹⁷⁷Lu-AMBA binding to untreated controls from 0.1-1.0 μM.

Interpretation: This demonstrates cross-talk between EGFR and/or HER2 and possibly GRP-R. This may indicate that the treatment is successfully targeting and that GRPR is not an alternative pathway for rescue; that the treatment is not successful and therefore there is no need to transactivate GRPR; or that GRP is mediating rescue, but there is no need for increased expression of GRPR. Results are recorded in Table 24 in Example LXVI.

### Example LXIX

### In Vitro Demonstration of the Effect of Dasatinib on GRP Receptor Binding in BT-474 Human Breast Cancer Cells

BT-474 cells were maintained and treated and binding assayed as in Example LXVI.

### Dasatinib Solutions:

### Stock: 1 mM in 100% DMSO

Working: (0.1-1.0 M) in complete growth medium

Results: Treatment with dasatinib resulted in a decrease in GRP receptor specific signal per cell in BT-474 breast cancer cells as indicated by a decrease in ¹⁷⁷Lu-AMBA binding of up to 10-15% of untreated control (0.1-1.0 M).

Interpretation: This demonstrates cross-talk between a member of the Src Family kinase and GRPR. The decrease could be indicative of effective targeting, but inability to switch to GRPR for support/rescue; or that treatment is not effective and therefore no need to switch to GRPR. Results are recorded Table 24 in Example LXVI.

### Example LXX

### In Vitro Demonstration of the Effect of Dasatinib on GRP Receptor Binding in T47D Human Breast Cancer Cells

T-47D cells were maintained and treated and binding assayed as in Example LXVI and LXVII and dasatinib solutions are as in Example LXIX.

Results: Treatment with dasatinib resulted in an increase in GRP receptor specific signal per cell in T-47D breast cancer cells as indicated by an increase in ¹⁷⁷Lu-AMBA binding of up to 500% of untreated control at (0.1-1.0 M).

Interpretation: This demonstrates cross-talk between a member of the Src Family kinase and GRPR; interpretation identical to Example LXVI. Results are recorded in Table 24 in Example LXVI.

### Example LXX1

### In Vitro Demonstration of the Effect of Dasatinib on GRP Receptor Binding in PC-3 Prostate Cancer Cells

PC-3 cells were maintained and treated and binding assayed as in Example LXVI and LXVIII and dasatinib solutions are as in Example LXIX.

Results: Treatment with dasatinib resulted in an increase in GRP receptor specific signal per cell in PC-3 prostate cancer cells as indicated by an increase in ¹⁷⁷Lu-AMBA binding of up to 250% of untreated control at (0.1-1.0 μM).

Interpretation: This demonstrates cross-talk between a member of the Src Family kinase and GRPR; interpretation identical to Example LXVI. Results are recorded in Table 24 in Example LXVI.

### Example LXXII

### In Vitro Demonstration of the Effect of Gefitinib on GRP Receptor Binding in BT-474 Human Breast Cancer Cells

BT-474 cells were maintained and treated and binding assayed as in Example LXVI.

### Gefitinib Solutions:

Stock: 1 mM stock in 0.5M H₃PO₄
Working: 0.1-10 μM in complete growth medium

Results: Treatment with gefitinib resulted in no change in GRP receptor specific signal per cell in BT-474 breast cancer cells as indicated by equivalent ¹⁷⁷Lu-AMBA binding to untreated controls from 0.1-10 μM.

Interpretation: This may demonstrate cross-talk between a member of the Src Family kinase and GRPR. This may indicate that the treatment is successfully targeting and that GRPR is not an alternative pathway for rescue; that the treatment is not successful and therefore there is no need to transactivate GRPR; or that GRP is mediating rescue, but there is no need for increased expression of GRPR. GRP has been reportedly linked to resistance to gefitinib (Liu X, et al., Exp Cell Res 2007, 313; 1361-72) by rescuing NSCLC cells exposed to gefitinib via downstream c-src mediated transactivation of Akt, a key EGFR-activated signaling pathway. However this may not require an increase in the expression of GRPR. Results are recorded in Table 24 in Example LXVI.

### Example LXXIII

### In Vitro Demonstration of the Effect of Gefitinib on GRP Receptor Binding in T47D Human Breast Cancer Cells

T-47D cells were maintained and treated and binding assayed as in Example LXVI and LXVII and gefitinib solutions are as in Example LXXII.

Results: Treatment with gefitinib resulted an increase in GRP receptor specific signal per cell in T-47D breast cancer cells as indicated by an increase in ¹⁷⁷Lu-AMBA binding of up to 20% of untreated controls (range 0.1-10 M).

Interpretation: This demonstrates crosstalk between EGFR and GRPR; interpretation as in Example LXVI. Results are recorded in Table 24 in Example LXVI.

### Example LXXIV

### In Vitro Demonstration of the Effect of Gefitinib on GRP Receptor Binding in PC-3 Prostate Cancer Cells

PC-3 cells were maintained and treated and binding assayed as in Example LXVI and LXVIII and gefitinib solutions are as in Example LXXII.

Results: Treatment with gefitinib resulted a decrease in GRP receptor specific signal per cell in PC-3 prostate cancer cells as indicated by a decrease in ¹⁷⁷Lu-AMBA binding of up to 20-40% of untreated controls (range 0.1-10 M).

Interpretation: This demonstrates crosstalk between EGFR and GRPR; interpretation as in Example LXIX. Results are recorded in Table 24 in Example LXVI.

### Example LXXV

### In Vitro Demonstration of the Effect of Imatinib on GRP Receptor Binding in BT-474 Human Breast Cancer Cells

BT-474 cells were maintained and treated and binding assayed as in Example LXVI.

### Imatinib Solutions:

Stock: 1 mM stock in 100 mM CH₃COOH
Working: 0.1-1.0 M in complete growth medium

Results: Treatment with imatinib resulted in no change in GRP receptor specific signal per cell in BT-474 breast cancer cells as indicated by equivalent ¹⁷⁷Lu-AMBA binding to untreated controls from 0.1-1.0 M.

Interpretation: This demonstrates cross-talk between multiple kinase inhibitors (Bcr-Abl tyrosine kinase; also inhibits PDGF and stem cell factor (SCF), Kit, and PDGFR) and GRPR; interpretation identical to Example LXVIII. Results are recorded in Table 24 in Example LXVI.

### Example LXXVI

### In Vitro Demonstration of the Effect of Imatinib on GRP Receptor Binding in T47D Human Breast Cancer Cells

T-47D cells were maintained and treated and binding assayed as in Example LXVI and LXVII imatinib solutions are as in Example LXXV.

Results: Treatment with imatinib resulted in resulted an increase in GRP receptor specific signal per cell in T-47D breast cancer cells as indicated by an increase in ¹⁷⁷Lu-AMBA binding of up to 15% of untreated controls (range 0.1-1.0 μM).

Interpretation: This demonstrates cross-talk between multiple kinase inhibitors (Bcr-Abl tyrosine kinase; also inhibits PDGF and stem cell factor (SCF), Kit, and PDGFR) and GRPR; interpretation identical to Example LXVI. Results are recorded in Table 24 in Example LXVI.

### Example LXXVII

### In Vitro Demonstration of the Effect of Imatinib in PC-3 Prostate Cancer Cells

PC-3 cells were maintained and treated and binding assayed as in Example LXVI and LXVIII and imatinib solutions are as in Example LXXV.

Results: Treatment with imatinib resulted in a decrease in GRP receptor specific signal per cell in PC-3 prostate cancer cells as indicated by a decrease in ¹⁷⁷Lu-AMBA binding of up to 15-25% of untreated controls (range 0.1-1.0 μM).

Interpretation: This demonstrates cross-talk between multiple kinase inhibitors (Bcr-Abl tyrosine kinase; also inhibits PDGF and stem cell factor (SCF), Kit, and PDGFR) and GRPR; interpretation identical to Example LXIX. Results are recorded in Table 24 in Example LXVI.

### Example LXXVIII

### In Vitro Demonstration of the Effect of Erlotinib on GRP Receptor Binding in BT-474 Human Breast Cancer Cells

BT-474 cells were maintained and treated and binding assayed as in Example LXVI.

### Erlotinib Solutions:

Stock: 1 mM stock in 100 mM CH₃COOH made in 95% ethanol
Working: 0.1-1.0 M in complete growth medium

Results: Treatment with erlotinib resulted in no change in GRP receptor specific signal per cell in BT-474 breast cancer cells as indicated by equivalent ¹⁷⁷Lu-AMBA binding to untreated controls from 0.1-1.0 M.

Interpretation: This demonstrates cross-talk between EGFR and GRP-R; interpretation identical to Example LXVIII. Results are recorded in Table 24 in Example LXVI.

### Example LXXIX

### In Vitro Demonstration of the Effect of Erlotinib on GRP Receptor Binding in T47D Human Breast Cancer Cells

T-47D cells were maintained and treated and binding assayed as in Example LXVI and LXVII and erlotinib solutions are as in Example LXXVIII.

Results: Treatment with erlotinib resulted in an increase in GRP receptor specific signal per cell in T-47D breast cancer cells as indicated by an increase in ¹⁷⁷Lu-AMBA binding of up to 5-20% of untreated controls (range 0.1-1.0 M).

Interpretation: This demonstrates cross-talk between EGFR and GRP-R; interpretation identical to Example LXVI. Results are recorded in Table 24 in Example LXVI.

### Example LXXX

### In Vitro Demonstration of the Effect of Erlotinib on GRP Receptor Binding in PC-3 Prostate Cancer Cells

PC-3 cells were maintained and treated and binding assayed as in Example LXVI LXVIII and erlotinib solutions are as in Example LXXVIII.

Results: Treatment with erlotinib resulted in a decrease in GRP receptor specific signal per cell in PC-3 prostate cancer cells as indicated by a decrease in ¹⁷⁷Lu-AMBA binding of up to 5-20% of untreated controls (range 0.1-1.0 M).

Interpretation: This demonstrates cross-talk between EGFR and GRP-R; interpretation identical to Example LXIX. Results are recorded in Table 24 in Example LXVI.

### Example LXXXI

### In Vitro Demonstration of the Effect of Sorafenib on GRP Receptor Binding in BT-474 Human Breast Cancer Cells

BT-474 cells were maintained and treated and binding assayed as in Example LXVI.

### Sorafenib Solutions:

Stock: 1 mM stock in 100 mM CH₃COOH made in 95% ethanol
Working: 0.1-1.0 μM in complete growth medium

Results: Treatment with sorafenib resulted in a decrease in GRP receptor specific signal per cell in BT-474 breast cancer cells as indicated by a decrease in ¹⁷⁷Lu-AMBA binding of up to 10-25% of untreated controls (range 0.1-1.0 μM).

Interpretation: This demonstrates crosstalk between multiple kinase inhibitors (PDGFRba/VEGFR 1,2,3/KIT, FLT3/EGF/Ras/Rafkinase) and GRPR; interpretation as in Example LXIX. Results are recorded in Table 24 in Example LXVI.

### Example LXXXII

### In Vitro Demonstration of the Effect of Sorafenib on GRP Receptor Binding in T47D Human Breast Cancer Cells

T-47D cells were maintained and treated and binding assayed as in Example LXVI and LXVII and sorafenib solutions are as in Example LXXXI.

Results: Treatment with sorafenib resulted in no change in GRP receptor specific signal per cell in T-47D breast cancer cells as indicated by equivalent ¹⁷⁷Lu-AMBA binding to untreated controls from 0.1-1.0 μM.

Interpretation: This demonstrates crosstalk between multiple kinase inhibitors (PDGFRba/VEGFR 1,2,3/KIT, FLT3/EGF/Ras/Rafkinase) and GRPR; interpretation as in Example LXVIII. Results are recorded in Table 24 in Example LXVI.

### Example LXXXIII

### In Vitro Demonstration of the Effect of Sorafenib on GRP Receptor Binding in PC-3 Prostate Cancer Cells

PC-3 cells were maintained and treated and binding assayed as in Example LXVI and LXVIII and sorafenib solutions are as in Example LXXXI.

Results: Treatment with sorafenib resulted in a decrease in GRP receptor specific signal per cell in PC-3 prostate cancer cells as indicated by a decrease in ¹⁷⁷Lu-AMBA binding of up to 10-20% of untreated controls (range 0.1-1.0 μM).

Interpretation: This demonstrates crosstalk between multiple kinase inhibitors (PDGFRba/VEGFR 1,2,3/KIT, FLT3/EGF/Ras/Rafkinase) and GRPR; interpretation as in Example LXIX. Results are recorded in Table 24 in Example LXVI.

### Example LXXXIV

### In Vitro Demonstration of the Effect of Sunitinib on GRP Receptor Binding in BT-474 Human Breast Cancer Cells

BT-474 cells were maintained and treated and binding assayed as in Example LXVI.

### Sunitinib Solutions:

Stock: 1 mM stock in 100 mM CH₃COOH made in 95%
Working: 0.1-1.0 μM in complete growth medium

Results: Treatment with sunitinib resulted in a decrease in GRP receptor specific signal per cell in BT-474 breast cancer cells as indicated by a decrease in ¹⁷⁷Lu-AMBA binding of up to 8% of untreated controls (range 0.1-1.0 μM).

Interpretation: This demonstrates crosstalk between multiple Kinase Inhibitors (EGFR, HER2, ErbB3; PDGFc and P3; stem cell factor receptor (KIT); FLT3; CSF-1R; neurotropic factor receptor (RET) and GRPR; interpretation identical to Example LXIX. Results are recorded in Table 24 in Example LXVI.

### Example LXXXV

### In Vitro Demonstration of the Effect of Sunitinib on GRP Receptor Binding in T47D Human Breast Cancer Cells

T-47D cells were maintained and treated and binding assayed as in Example LXVI and LXVII and sunitinib solutions are as in Example LXXXIV.

Results: Treatment with sunitinib resulted in an increase in GRP receptor specific signal per cell in T-47D breast cancer cells as indicated by an increase in ¹⁷⁷Lu-AMBA binding of up to 13% of untreated controls (range 0.1-1.0 μM).

Interpretation: This demonstrates crosstalk between multiple Kinase Inhibitors (EGFR, HER2, ErbB3; PDGFc and P3; stem cell factor receptor (KIT); FLT3; CSF-1R; neurotropic factor receptor (RET) and GRPR; interpretation identical to Example LXVI. Results are recorded in Table 24 in Example LXVI.

### Example LXXXVI

### In Vitro Demonstration of the Effect of Sunitinib on GRP Receptor Binding in PC-3 Prostate Cancer Cells

PC-3 cells were maintained and treated and binding assayed as in Example LXVI and LXVIII and sunitinib solutions are as in Example LXXXIV.

Results: Treatment with sunitinib resulted in an increase in GRP receptor specific signal per cell in PC-3 prostate cancer cells as indicated by an increase in ¹⁷⁷Lu-AMBA binding of up to 10% of untreated controls (range 0.1-1.0 μM).

Interpretation: This demonstrates crosstalk between multiple Kinase Inhibitors (EGFR, HER2, ErbB3; PDGFc and P3; stem cell factor receptor (KIT); FLT3; CSF-1R; neurotropic factor receptor (RET) and GRPR; interpretation identical to Example LXVI.

Results are recorded in Table 24 in Example LXVI.

### Example LXXXVII

### In Vitro Demonstration of the Effect of Anastrozole on GRP Receptor Binding in BT-474 Human Breast Cancer Cells

BT-474 cells were maintained and treated and binding assayed as in Example LXVI.

### Anastrozole Solutions:

Stock: 1 mM stock in 100% ethanol
Working: 0.1-1.0 μM in complete growth medium

Results: Treatment with anastrozole resulted in no change in GRP receptor specific signal per cell in BT-474 breast cancer cells as indicated by equivalent ¹⁷⁷Lu-AMBA binding to untreated controls from 0.1-1.0 μM.

Interpretation: This demonstrates crosstalk between the Estrogen receptor and GRPR; interpretation identical to Example LXVIII. Results are recorded in Table 24 in Example LXVI.

### Example LXXXVIII

### In Vitro Demonstration of the Effect of Anastrozole on GRP Receptor Binding in T47D Human Breast Cancer Cells

T-47D cells were maintained and treated and binding assayed as in Example LXVI and LXVII and anastrozole solutions are as in Example LXXXVII.

Results: Treatment with anastrozole resulted in a decrease in GRP receptor specific signal per cell in T-47D breast cancer cells as indicated by a decrease in ¹⁷⁷Lu-AMBA binding of up to 10% of untreated controls (range 0.1-1.0 μM).

Interpretation: This demonstrates crosstalk between the Estrogen receptor and GRPR; interpretation identical to Example LXIX. Results are recorded in Table 24 in Example LXVI.

### Example LXXXIX

### In Vitro Demonstration of the Effect of Anastrozole on GRP Receptor Binding in PC-3 Prostate Cancer Cells

PC-3 cells were maintained and treated and binding assayed as in Example LXVI and LXVIII and anastrozole solutions are as in Example LXXXVII.

Results: Treatment with anastrozole resulted in a decrease in GRP receptor specific signal per cell in PC-3 prostate cancer cells as indicated by a decrease in ¹⁷⁷Lu-AMBA binding of up to 10% of untreated controls (range 0.1-1.0 μM).

Interpretation: This demonstrates crosstalk between the Estrogen receptor and GRPR; interpretation identical to Example LXIX. Results are recorded in Table 24 in Example LXVI.

### Example XC

### In Vitro Demonstration of the Effect of Bortezomib on GRP Receptor Binding in BT-474 Human Breast Cancer Cells

BT-474 cells were maintained and treated and binding assayed as in Example LXVI.

### Bortezomib Solutions:

Stock: 1 mM stock in 100% DMSO
Working: 0.1-1.0 μM in complete growth medium

Results: Treatment with bortezomib resulted in a decrease in GRP receptor specific signal per cell in BT-474 breast cancer cells as indicated by a decrease in ¹⁷⁷Lu-AMBA binding of up to 50% of untreated controls (range 0.1-1.0 μM).

Interpretation: This demonstrates crosstalk between downstream signaling in the ubiquitin pathway and GRPR; interpretation identical to Example LXIX. Results are recorded in Table 24 in Example LXVI.

### Example XCI

### In Vitro Demonstration of the Effect of Bortezomib on GRP Receptor Binding in T47D Human Breast Cancer Cells

T-47D cells were maintained and treated and binding assayed as in Example LXVI and LXVII and bortezomib solutions are as in Example XC.

Results: Treatment with bortezomib resulted in a decrease in GRP receptor specific signal per cell in T-47D breast cancer cells as indicated by a decrease in ¹⁷⁷Lu-AMBA binding of up to 50% of untreated controls (range 0.1-1.0 M).

Interpretation: This demonstrates crosstalk between downstream signaling in the ubiquitin pathway and GRPR; interpretation identical to Example LXIX. Results are recorded in Table 24 in Example LXVI.

### Example XCII

### In Vitro Demonstration of the Effect of Bortezomib on GRP Receptor Binding in PC-3 Prostate Cancer Cells

PC-3 cells were maintained and treated and binding assayed as in Example LXVI and LXVIII and bortezomib solutions are as in Example XC.

Results: Treatment with bortezomib resulted in a decrease in GRP receptor specific signal per cell in PC-3 prostate cancer cells as indicated by a decrease in ¹⁷⁷Lu-AMBA binding of up to 60% of untreated controls (range 0.1-1.0 μM).

Interpretation: This demonstrates crosstalk between downstream signaling in the ubiquitin pathway and GRPR; interpretation identical to Example LXIX. Results are recorded in Table 24 in Example LXVI.

### Example XCIII

### In Vitro Demonstration of the Effect of 4-OH Tamoxifen on GRP Receptor Binding in BT-474 Human Breast Cancer Cells

BT-474 cells were maintained and treated and binding assayed as in Example LXVI.

### 4-OH Tamoxifen Solutions:

Stock: 1 mM in 95% ethanol
Working: 0.001-0.1 μM in complete growth medium

Results: Treatment with 4-OH Tamoxifen resulted in a decrease in GRP receptor specific signal per cell in BT-474 breast cancer cells as indicated by a decrease in ¹⁷⁷Lu-AMBA binding of up to 73% of untreated controls (range 0.001-0.1 M).

Interpretation: This demonstrates crosstalk between the ER and GRPR; interpretation identical to Example LXIX. Results are recorded in Table 25, below:

TABLE 25

| | | |
|---|---|---|
| Compounds | Prostate Cancer | Breast Cancer |

| Tested | PC-3 | T47D | BT-474 |
|---|---|---|---|
| 4-OH-Tamoxifen | 0 | −35% | −73% |
| β2-estradiol | 0 | 0 | 47% |
| 4-OH-Tamoxifen + | 0 | −27% | 0 |
| β2-estradiol | | | |
| ] | | | |

### Example XCIV

### In Vitro Demonstration of the Effect of 4-OH Tamoxifen on GRP Receptor Binding in T47D Human Breast Cancer Cells

T-47D cells were maintained and treated and binding assayed as in Example LXVI and LXVII and 4-OH Tamoxifen solutions are as in Example XCIII.

Results: Treatment with 4-OH Tamoxifen resulted in a decrease in GRP receptor specific signal per cell in T-47D breast cancer cells as indicated by a decrease in ¹⁷⁷Lu-AMBA binding of up to 35% of untreated controls (range 0.001-0.1 μM).

Interpretation: This demonstrates crosstalk between the ER and GRPR; interpretation identical to Example LXIX. Results are recorded in Table 25 in Example XCIII.

### Example XCV

### In Vitro Demonstration of the Effect of 4-OH Tamoxifen on GRP Receptor Binding in PC-3 Prostate Cancer Cells

PC-3 cells were maintained and treated and binding assayed as in Example LXVI and LXVIII and 4-OH Tamoxifen solutions are as in Example XCIII.

Results: Treatment with Tamoxifen (4-OH TMX) resulted in no change in GRP receptor specific signal per cell in PC-3 prostate cancer cells as indicated by equivalent ¹⁷⁷Lu-AMBA binding to untreated controls from range 0.001-0.1 μM.

Interpretation: This demonstrates crosstalk between the ER and GRPR; interpretation identical to Example LXVIII. Results are recorded in Table 25 in Example XCIII.

### Example XCVI

### In Vitro Demonstration of the Effect of 4-OH Tamoxifen plus β2-Estradiol on GRP Receptor Binding in BT-474 Human Breast Cancer Cells

BT-474 cells were maintained and treated and binding assayed as in Example LXVI with the exception of addition of 132-estradiol (see below).

4-OH Tamoxifen solutions:
Stock: 1 mM in 95% ethanol
Working: 0.001-0.1 μM in complete growth medium

### β2-Estradiol Solutions:

Stock: 1 mM in 95% ethanol
Working: 1 nM in complete growth medium

Treatment: BT-474 cells were plated on Day 0 and treated on Days 1 and 2 (D_{T}1, D_{T}2). The medium was removed and replaced with either fresh growth medium (control); or growth medium on D_{T}1, followed by 1 nM J32-estradiol on D_{T}2; or 4-OH Tamoxifen on D_{T}1 followed by 1 nM J32-estradiol on D_{T}2.

Results: Treatment with 132-estradiol increased GRP receptor specific signal per cell in BT-474 breast cancer cells as indicated by an increase in ¹⁷⁷Lu-AMBA binding 47% over control values. Treatment with 4-OH Tamoxifen followed by J32-estradiol did not change the values obtained with 4-OH Tamoxifen treatment alone (see Example XCIII).

Interpretation: This demonstrates crosstalk between the ER and GRPR; interpretation identical to Example LXVI. Results are recorded in Table 25 in Example XCIII.

### Example XCVII

### In Vitro Demonstration of the Effect of 4-OH Tamoxifen Plus β2-Estradiol and β2-Estradiol Alone on GRP Receptor Binding in T47D Human Breast Cancer Cells

T47D cells were maintained and treated and binding assayed as in Example LXVI and LXVII, and XCVI.

Results: Treatment with β2-estradiol did not change GRP receptor specific signal per cell in T47D breast cancer cells. Treatment with Tamoxifen followed by β2-estradiol resulted in a decrease in GRP-R expression per cell in T-47D breast cancer cells by 27%, 8% less than with Tamoxifen alone (Example XCIV).

Interpretation: This demonstrates crosstalk between the ER and GRPR; interpretation identical to Example LXVIII. Results are recorded in Table 25 in Example XCIII.

### Example XCVIII

### In Vitro Demonstration of the Effect of 4-OH Tamoxifen Plus β2-Estradiol on GRP Receptor Binding in PC-3 Prostate Cancer Cells

PC-3 cells were maintained and treated and binding assayed as in Example LXVI and LXVIII, and XCVI.

Results: Treatment with 32-estradiol or 4-OH Tamoxifen followed by 12-estradiol resulted in no change in GRP receptor specific signal per cell in PC-3 prostate cancer cells as indicated by equivalent ¹⁷⁷Lu-AMBA binding to untreated controls from range 0.001-0.1 μM.

Interpretation: This demonstrates crosstalk between the ER and GRPR; interpretation identical to Example LXVIII. Results are recorded in Table 25 in Example XCIII.

### Example XCIX

### In Vitro Demonstration of the Effect of Dasatinib on GRP Receptor Binding and Glucose uptake in PC-3 prostate Cancer Cells (Using a Single Plate Assay Method)

Method: PC-3 (human prostate adenocarcinoma, bone metastasis, AR-, ER) were obtained from the American Type Culture Collection. The growth medium for PC-3 cells is RPMI 1640 (10-041-CM, Mediatech, Inc), supplemented with 10% heat inactivated FBS (Hyclone, SH30070.03) and PSF-1× (as in Example LXVI). The cells were plated on Day 0 (7.5k/well 96-well PE Scintiplate). Approximately 24 h after plating, treatment Days 1 and 2 (D_{T}1, D_{T}2), the medium was removed and replaced with fresh growth medium (control), or growth medium with the addition of Dasatinib (0-0.1 μM). On day3, ¹⁷⁷Lu-AMBA binding assay was carried out as described earlier. To determine the cell numbers and the proliferation, 100 uL of 10% CCK-F solution in DPBS (Molecular technologies) was added to the same plate, incubated for 30 min at 37° C. and the relative fluorescence (RFU) measured at 535 nm. Base on the cell numbers and the ¹⁷⁷Lu-binding, the Bmax (fmoles bound/million cells) was calculated, and the results compared with the control.

For the glucose uptake study, a fluorescently labeled 2-deoxyglucose derivative (2-NBDG) was used. After the dasatinib treatment as described above, the cells were incubated with 2-NBDG (200 uM) in binding buffer for 2 h at 37° C. and measured RFU at 530 nm. The cell numbers were determined using CCK-F as described above. Based on the cell numbers Bmax for glucose uptake was determined and compared with the control.

As expected there was a significant decrease in the cell proliferation, the inhibition of growth being as much as 33% at 100 nM Dasatinib. Suprisingly, the uptake of ¹⁷⁷Lu-AMBA increased from an average Bₘₐₓ of 266 fmoles/million cells with out Dasatinib treatment (control) to as high as 470 fmoles/million cells) at 100 nM Dasatinib, an increase of 76.7%. The glucose uptake was decreased by as much as 67.8% at 100 nM dasatinib.

Effect of Dasatinib treatment on the relative change in proliferation of, and
¹⁷⁷Lu-AMBA uptake in prostate cancer (PC3) cells (N = 4)

| | | ¹⁷⁷Lu- | | |
|---|---|---|---|---|
| | | AMBA | | |
| | | uptake | | |
| | | (Bₘₐₓ | | |
| Dasatinib | | fmoles/ | % change in | |
| treatment | % Change in | million | ¹⁷⁷Lu-AMBA | % Change in 2- |
| (nM) | Proliferation | cells) | uptake | NBDG uptake |
| 0 | 0.00 | 266 (±23) | 0.00 | 0.00 |
| 1 | −4.3 (±1.0) | 300 (±40) | 12.3 (±5.8) | −4.3 (±4.6) |
| 10 | −11.2 (±5.9) | 387 (±46) | 45.4 (±8.1) | −62.9 (±7.1) |
| 100 | −33.3 (±11.4) | 470 (±51) | 76.7 (±5.0) | −67.8 (±15.8) |

The results demonstrate the ability of the GRP receptor activity to document the effect of Dasatinib treatment targeted towards the Src receptor family.

### Example C

### In Vitro Demonstration of the Effect of 4-hydroxytamoxifen on GRP Receptor Binding and Glucose Uptake in ZR-75-1 Human Breast Cancer Cells

Method: ZR-75-1 breast cancer cell line (human ductal carcinoma with estrogen receptor) was obtained from the American Type Culture Collection (ATCC). The cells were cultured in complete growth medium, RPMI-1640 medium from ATCC (Cat No: 30-2001) supplemented with 10% heat inactivated FBS (hyclone, SH30070.03) in T-150 CellBind Tissue culture flasks (Corning) and maintained in a humidified atmosphere containing 5% CO₂/95% air at 37° C. The cells were passaged routinely by using 0.25% trypsin/EDTA from Mediatech, Inc. (Cat No: 25-053-cl).

ZR-75-1 cells were plated on Day 0 (40k/well 96-well PE Scintiplate). Approximately 48 h after plating, treatment, Days 2 and 3 (D_{T}2, D_{T}3), the medium was removed and replaced with fresh growth medium (control), or growth medium with the addition of 4-hydroxytamoxifen (0-0.1 M). On Day-4, ¹⁷⁷Lu-AMBA binding assay was carried out as described earlier. To determine the cell numbers and the proliferation, 100 uL of 10% CCK-F solution in DPBS (Molecular technologies) was added to the same plate, incubated for 30 min at 37° C. and the relative fluorescence (RFU) measured at 535 nm. Based on the cell numbers and the ¹⁷⁷Lu-binding, the Bmax (fmoles bound/million cells) was calculated.

For the glucose uptake study, a fluorescently labeled2-deoxyglucose derivative (2-NBDG) was used. After the 4-hydroxytamoxifen treatment as described above, the cells were incubated with 2-NBDG (200 uM) in binding buffer for 2 h at 37° C. and measured RFU at 530 nm. The cell numbers were determined with CCK-F treatment as described above.

| | % Change in ¹⁷⁷Lu-AMBA uptake on a viable | | |
|---|---|---|---|
| ZR-75-1 cells | cell basis in ZR-75-1 Breast cancer cells (N = 3) | | |
| 4-Hydroxy- | | % change in | |
| Tamoxifen | % Change in | ¹⁷⁷Lu-AMBA | % change in 2- |
| treatment [nM] | proliferation | uptake | NBDG uptake |
| 0 | 0 | 0.00 | 0 |
| 1 | −5.9 (±3.7) | −42.6 (±7.1) | −23.2 (±30.5) |
| 10 | −6.4 (±4.2) | −57.3 (±8.4) | −16.1 (±8.8) |
| 100 | −8.5 (±1.5) | −69.2 (±4.9) | −13.0 (±19.9) |

There was little change in the cell proliferation tested at these concentrations. The relative uptake of ¹⁷⁷Lu-AMBA in ZR-75-1 cells was significantly decreased by 40-70% after treatment with 1-100 nM 4-OH Tamoxifen whereas there was a moderate decrease (˜20%) in glycolysis as demonstrated by 2-NBDG uptake The results demonstrate the ability of the GRP receptor activity to document the effect of tamoxifen treatment targeted towards the estrogen receptor.

### Example

### CI-In Vitro Demonstration of the Longterm (2-12 days) Effect of 4-Hydroxytamoxifen on GRP Receptor Binding in ZR-75-1 Human Breast Cancer Cells

Method:

1. Effect of 4-OH Tamoxifen treatment on GRP uptake on day-2

ZR-75-1 breast cancer cell lines were cultured as described above. The 2-day study with 4-OH Tam followed by ¹⁷⁷Lu-AMBA uptake was carried out as above.

2. Effect of 4-OH Tamoxifen on Grp or Glucose Uptake in ZR-75-1 Cells on Day 5:

The ZR-75-1 cells were seeded at 15K per well onto two 96-well scintiplates on the same day as 2 days study. The treatment was performed with 100 nM 4-OH tamoxifen everyday from day 2 to day 5. The effect of 4-OH Tamoxifen on GRP-R or glucose uptake in ZR-75-1 cells was determined on day 6 by the same method as effect of 2 days 4-OH tamoxifen study.

3. Effect of 4-OH Tamoxifen on GRP or Glucose Uptake in ZR-75-1 Cells on Day 11:

The ZR-75-1 cells were cultured in T-75 TC flasks in the same day when this study started, and the cells were treated with or without 100 nM 4-OH tamoxifen treatment everyday from day 2 to day 5. On day6, the cells were seeded at 15K per well onto two 96-well scintiplates and the treatment was performed with 100 nM 4-OH tamoxifen everyday until day 11. The effect of 4-OH Tamoxifen on GRP-R or glucose uptake in ZR-75-1 cells was determined on day 12 by the same method applied previously.

| Days of | % Change in | % change in 177Lu- | Number of | % Change in 2- | Number of |
|---|---|---|---|---|---|
| Treatment | Proliferation | AMBA Uptake | Assay | NBDG | assay |
| 0 | 0 | 0.0 | | 0 | 0 |
| 2 | −7.2 (±3.8 | −65.9 (±9.7) | 5 | −13.01 (±9.8) | 3 |
| 5 | −11.3 (±8.3) | −77.4 (+1.6) | 2 | −47.5 | 1 |
| 12 | −28.0 (±14.1) | −72.3 (±14.6) | 2 | 14.5 | 1 |

The results showed that the long-term treatment of ZR-75-1 with 4-OH Tamoxifen causes a drop in proliferation of ZR-75-1 cells of 28% inhibition seen by 12 day. A significant reduction in ¹⁷⁷Lu-AMBA uptake was seen with smaller changes in 2-NBDG uptake in response to treatment. The results demonstrate the ability of the GRP receptor activity to document the effect of tamoxifen treatment targeted towards the estrogen receptor. ⁶⁷Ga-AMBA is better suited than ¹⁸F-FDG to monitor the progress of such treatment.

### Example CII

### Preparation and Characterization of Ga-AMBA (Ga-L70)

(a) Synthesis of AMBA Ligand:

AMBA [(DO3A-CH₂CO-G-(4-aminobenzoyl)-QWAVGHLM-NH₂), DO3A=(1,4,7,10-tetraaza-4,7,10-tris(carboxymethyl)-cyclododecyl)-acetyl] was synthesized using solid phase peptide synthesis chemistry, as described in U.S. Pat. No. 7,226,577 (Cappelletti et al)

The structure of the ligand is shown below.

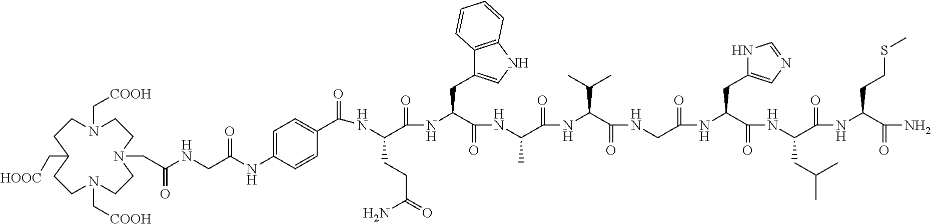 [SMILES: CSCC[C@H](NC(=O)[C@H](CC(C)C)NC(=O)[C@H](Cc1cnc[nH]1)NC(=O)CNC(=O)[C@@H](NC(=O)[C@H](C)NC(=O)[C@H](Cc1c[nH]c2ccccc12)NC(=O)[C@H](CCC(N)=O)NC(=O)c1ccc(NC(=O)CNC(=O)CN2CCN(CC(=O)O)CCC(CC(=O)O)CCN(CC(=O)O)CC2)cc1)C(C)C)C(N)=O]

(b) Synthesis of Non-Radioactive Ga-AMBA Standard:

A sample of non-radioactive Ga-AMBA was prepared by mixing AMBA ligand (25 g) and selenomethionine (200 μg) in 0.2 mL of 0.2M NaOAc buffer (pH 4.8) with 2 μg of gallium (Gallium AAS standard solution). This represents a Ga-to-ligand ratio of 1.8 to 1. An excess of Ga was used to consume all AMBA ligand and render subsequent purification more straightforward. The mixture was heated at 100° C. for 10 min. After the coordination, 20 L of 10 mM EDTA was added into the reaction solution to chelate any remaining free gallium. The Ga-AMBA reaction solution was injected into an HPLC for LC/MS analysis using the conditions listed below.

Column: Zorbax Bonus-RP embedded amide C14 (250 mm×4.6 mm, size 5 m). Solvents: A: H₂O/0.1% formic acid/0.01% TFA (v/v); B: Acetonitrile/0.1% formic acid/0.01% TFA (v/v). The gradient is listed in Table 26 below. Flow rate: 0.6 mL/min. Column temperature: 37° C.

HPLC Gradient used for Ga-AMBA LC/MS Analysis:

TABLE 26

| | | A (%) | B (%) |
|---|---|---|---|
| 0-5 | min | 95 | 5 |
| 15 | min | 82 | 18 |
| 40 | min | 75 | 25 |
| 45-50 | min | 20 | 80 |
| 55 | min | 95 | 5 |
| Post run 10 | min | 95 | 5 |

Under the HPLC conditions used for the LC/MS analysis, the retention time of Ga-AMBA was ˜24.8 min. Naturally occurring gallium is a mixture of two non-radioactive isotopes, Ga-69 and Ga-71 with an abundance of 60.11% and 39.89%, respectively. A good agreement between the measured and the expected isotopic masses of Ga-AMBA was obtained.

| | |
|---|---|
| Expected | Measured (M + H)⁺ |

| | ⁶⁹Ga | ⁷¹Ga | ⁶⁹Ga | ⁷¹Ga |
|---|---|---|---|---|
| Isotopic mass | 1568.67 | 1570.67 | 1568.5 | 1570.4 |

A second non-radioactive Ga-AMBA standard was prepared by treating 10 mg “as is” AMBA dissolved in 10 mL of 0.2 M, pH 4.8 NaOAc buffer with 0.8 mg of gallium (Gallium AAS standard solution). In this reaction, the Ga-to-ligand ratio was ˜2 to 1. Excess Ga was used to consume all AMBA during chelation, in order to facilitate subsequent purification of the Ga-AMBA complex. The mixture was heated at 100° C. for 20 min. After the coordination, 2 mL of 10 mM EDTA was added into the reaction solution to chelate any remaining free gallium.

The Ga-AMBA was purified by preparative HPLC using the HPLC conditions listed below.

HPLC conditions for Ga-AMBA purification

| | |
|---|---|
| Column | YMC C18; 250 × 10 mm; 10 μm |
| Solvents | A: H₂O/0.1% TFA (v:v); B: ACN/0.1% |
| | TFA (v:v) |
| Gradient | 10-50% B over 40 min |
| Flow rate | 15 mL/min |
| Retention time of Ga-AMBA | 16.23 min |

Preparative HPLC Conditions for Large Scale Ga-AMBA Purification HPLC Conditions for Ga-AMBA Purification:

The Ga-AMBA peak was collected and lyophilized. An 8.4 mg sample of Ga-AMBA was obtained, and the results of elemental analysis were consistent with a formula weight of 2037.6 (3TFA. 7H₂O)

Elemental Analysis Results for Ga-AMBA 3TFA.7H₂O

Ga-AMBA elemental analysis results

| | | FW | C % | H % | N % |
|---|---|---|---|---|---|
| Anal. | | | 43.48 | 5.55 | 13.07 |
| Calcd. | C₇₂H₁₁₃N₁₉O₃SGaF₉ | 2037.6 | 43.62 | 5.59 | 13.06 |
| | (3TFA•7H₂O) | | | | |

(c) Preparation of ⁶⁷Ga-AMBA:

⁶⁷Ga-AMBA was prepared using a method that was similar to the one used for ¹⁷⁷Lu-AMBA as described herein and in co-pending U.S. application U.S. Ser. No. 11/751,337. A solution of AMBA (6 μg of anhydrous ligand, 4 nmol) and selenomethionine (0.05 mg) in 50 μL of 0.2M, pH 4.8 NaOAc buffer was mixed with 1.7 mCi of ⁶⁷GaCl₃ (Nordion). The reaction vial was heated in a 100° C. heating block for 10 min, and then cooled for ˜2 min in a room-temperature water bath. After cooling, 0.1 mL of a 9:1 mixture of Bacteriostatic 0.9% Sodium Chloride Injection USP and ASCOR L500®Ascorbic Acid Injection USP was added to the vial, followed by 20 L of 10 mM EDTA to coordinate any remaining free radionuclide. The RCP of the unpurified reaction mixture was 90.1%. An aliquot of the reaction mixture was HPLC purified to remove excess ligand, using the following HPLC system. Column: Zorbax Bonus-RP embedded amide C14 (250 mm×4.6 mm, size 5 μm, Agilent). Column temperature: 37° C., Flow rate: 1.5 mL per minute. Gradient: Mobile phase A=H₂O, B=H₂O with 0.1% trifluoroacetic acid (TFA) (v/v) and 30 mM (NH₄)₂SO₄. (3.96 g/L), C=Methanol (MeOH), and D=Acetonitrile (ACN).

Mobile Phase Gradient for ⁶⁷Ga-AMBA Analysis

| Time (min) | % A | % B | % C | % D |
|---|---|---|---|---|
| 0 | 30 | 60 | 5 | 5 |
| 5 | 14 | 60 | 13 | 13 |
| 37 | 14 | 60 | 13 | 13 |
| 40 | 0 | 60 | 20 | 20 |
| 45 | 0 | 60 | 20 | 20 |
| 46 | 30 | 60 | 5 | 5 |

The desired fractions were collected into 1 mL of Bacteriostatic saline/ASCOR L500 (9:1) containing 0.1% HSA. The organics were removed using a speed vacuum and the final product was diluted to 0.1 mCi/mL using a 9:1 mixture of Bacteriostatic Saline/ASCOR L500 containing 0.1% HSA. The final RCP was 99.8%. The average specific activity of ⁶⁷Ga-AMBA at the time of preparation was ˜20 Ci/mole. The retention time of the Ga-AMBA standard (26.2 min) was the same as that of ⁶⁷Ga-AMBA (26.4 min), when the detector offsets were taken into consideration.

(d) In Vitro Binding of ⁶⁷Ga-AMBA to PC-3 Tumor Cells.

Human prostate cancer (PC-3) cells were obtained from the American Type Culture Collection (ATCC, Lot #3250) and cultured in RPMI-1640 containing L-glutamine and 25 mM HEPES (from Cellgro, cat#10-041-CM) in tissue culture flasks (Corning). This growth medium was supplemented with 10% heat inactivated FBS (Hyclone, SH30070.03), and fungizone (0.25 μg/mL). All cultures were maintained in a humidified atmosphere containing 5% CO₂ at 37° C., and passaged routinely using 0.25% trypsin/EDTA (VWR, Cat #45000-664). Cells were plated at a concentration of 20×10³/well in 96-well white/clear bottom tissue culture plates (Falcon Optilux-I). When confluent, the total number of cells was determined to be approximately 25-30×10³ cells/well. Plates were used for binding studies on day 2 post-plating. Binding buffer was RPMI-1640 supplemented with 0.2% BSA (w/v), 0.5 mM PMSF (AEBSF), Bacitracin (Sigma, lot #60K082; 50 mg/500 ml), pH 7.2 (RPMI-1640 was obtained from Cellgro, Cat#10-041-Cm, Lot #10041059).

A 96-well plate assay was employed to determine the EC₅₀ of nonradioactive Ga-AMBA to inhibit the binding of ⁶⁷Ga-AMBA to GRP-R. All compounds tested were dissolved in binding buffer and appropriate dilutions were also done in binding buffer. For the assay, AMBA ligand, or non-radioactive metal complexes Lu-AMBA or Ga-AMBA at concentrations ranging from 1.0×10⁻⁹ M to 50×10⁻⁹ M, was co-incubated with ⁶⁷Ga-AMBA (0.035 Ci, 1.5 Ci/mL) in 75 L total volume. These studies were conducted with an assay volume of 75 μL per well, using triplicate wells for each data point. After the addition of the appropriate solutions, plates were incubated for 1 hour at 4° C. The binding study was carried out at 4° C. to prevent internalization of the ligand-receptor complex. Incubation was ended by the addition of 200 L of ice-cold incubation buffer. Plates were washed five times and blotted dry. The cell bound radioactivity was detected using the LKB CompuGamma counter. The data were analyzed by GraphPad Prizm software to obtain the ‘Effective concentration’ needed to inhibit 50% binding (EC₅₀ value).

Saturation Binding Studies of ⁶⁷Ga-AMBA Using a Plate Assay:

For the saturation binding of ⁶⁷Ga-AMBA, the following general procedure was used: PC-3 cells were plated as described above, and when confluent, to each well was then added 75 L of carrier added ⁶⁷Ga-AMBA [stock: 20 μCi/mL, 50 nM] in triplicate at different concentrations [0-50 nM], and the cells incubated at 4° C. for 1 h. In a parallel experiment, the cells were incubated with various concentrations of ⁶⁷Ga-AMBA in the presence of a large excess of cold Ga-AMBA (1 μM) to determine any non-specific binding (NSB). After washing off the unbound radioactivity, total radioactivity bound to the cells was determined. The data were analyzed using GraphPad Prism software to obtain the K_{d} and the Bₘₐₓ values. The receptor numbers were calculated from the Bₘₐₓ values.

Internalization and Efflux Assay:

PC-3 cells were incubated with ⁶⁷Ga-AMBA (100,000 cpm, 75 L, 1 μCi/mL) in binding buffer (total assay volume 75 L) for 40 min at 37° C. Incubations were stopped by the addition of 200 L of ice-cold binding buffer, and the unbound radioactivity removed by washing (4×) with binding buffer (4° C.). To remove cell surface-bound radioligand, the cells were incubated with 0.2 M acetic acid in saline (pH 2.8) for 4 min at 0-5° C. (ice-bath). The solutions (acid wash media) from each well were collected individually. The acid wash was repeated again, and the combined solutions were counted in a gamma counter to determine the surface bound radioactivity. The cells were then lysed by treating with 0.5N NaOH (100 μL/well) for 3 min at room temperature. The solutions from each well were collected individually. The incubation with 0.5 N NaOH was repeated again. The combined solutions were counted to determine the amount of radioactive material that was internalized by PC-3 cells. All samples were analyzed in a gamma counter.

For the efflux studies, after incubating the PC-3 cells with ⁶⁷Ga-AMBA (75 L, 1 μCi/mL) for 40 min at 37° C., the unbound material was washed off (4×) with cold binding buffer (4° C.). Fresh binding media (200 L) was added to each well, and the cells incubated for up to 3 h. At 0, 1, 2 and 3 h, the radioactivity in the medium (observed efflux), surface bound (acid wash) and internalized (lysate) were determined as described above.

Since the observed efflux in the medium may contain radioactivity released from the cell surface, the percentage of true efflux was calculated from the radioactivity that remained internalized in cells at each time point using the following equation:

$$Efflux ( \% ) at 2 h = \frac{[ \% Internalized at t = 0 min ] - [ \% Internalized at 2 h ]}{[ \% Internalized at t = 0 ]} \times 100$$

AMBA was shown to compete equally well with both ¹⁷⁷Lu-AMBA and ⁶⁷Ga-AMBA, the EC₅₀ being 2.95 (±0.28) and 2.89 (±0.18) nM respectively. Similarly, both Lu- and Ga-chelates of AMBA compete and cross-compete with ⁶⁷Ga-AMBA and ¹⁷⁷Lu-AMBA at 1.0-1.5 nM [EC₅₀].

Comparison of competition binding of AMBA ligand and non-radioactive metal complexes with radio-labeled AMBA to PC-3 cells

| | Competition with | Competition with |
|---|---|---|
| Competitor | ¹⁷⁷Lu-AMBA EC₅₀ [nM]* | ⁶⁷Ga-AMBA EC₅₀ [nM]* |
| AMBA | 2.95 (±0.28) | 2.89 (±0.18) |
| Lu-AMBA | 1.47 (±0.11) | 1.28 (±0.24) |
| Ga-AMBA | 1.15 (+0.01) | 1.00 (±0.08) |

*Results are the average values of three different experiments. # EC₅₀ values are not equal to kD because max concentration of Ga-AMBA insufficient to achieve saturation.

Both Lu- and Ga-AMBA were found to compete and cross-compete with ⁶⁷Ga-AMBA and ¹⁷⁷Lu-AMBA even at a lower concentration than the metal-free AMBA. For example, the EC₅₀ values for the metal chelates (Lu-AMBA and Ga-AMBA) were found to be in the 1.0-1.5 nM range, while the metal free AMBA consistently showed a slightly higher EC₅₀ value (2.89-2.95 nM).

The binding affinity was determined by saturation binding of carrier-added ⁶⁷Ga-AMBA to GRP-R in PC-3. The data were analyzed by Prizm software to obtain the affinity (kD) and the binding capacity (Bₘₐₓ). From the Bₘₐₓ value, the receptor density was calculated.

The results show that the affinity of ⁶⁷Ga-AMBA [kD 0.46±0.07 nM] is similar to that of ¹⁷⁷Lu-AMBA (kD 0.44±0.08 nM) for the GRP-R in PC-3 cells. Similarly, the Bₘₐₓ of 402 fmoles/million cells and the receptor numbers 242,000/cell are similar to those obtained from ¹⁷⁷Lu-AMBA study (Bₘₐₓ408 fmoles/million cells, and receptors 245,000/cell).

Comparison of Binding of ⁶⁷Ga-AMBA and ¹⁷⁷Lu-AMBA by PC-3 Cells

| | | Binding capacity | |
|---|---|---|---|
| | Affinity to GRP-R | (Bₘₐₓ)* fmoles/ | Receptors/cell |
| Ligand | (kD in nM)* | 1 × 10⁶ cells | (×1000)* |
| ⁶⁷Ga-AMBA | 0.46 (±0.07) | 402 (±67)# | 242 (±46)# |
| ¹⁷⁷Lu-AMBA | 0.44 (±0.08) | 408 (±6.0) | 245 (±3.0) |

*Results are the average values of three different experiments;
#High standard deviation due to one of the three values being an outlier)

Following pre-incubation of ⁶⁷Ga-AMBA with PC-3 cells, 78.4% of the total cell bound radioactivity, was shown to be internalized, and the surface bound amounted to 20.9%. Almost all of the internalized activity remained internalized even after 3 h. The calculated efflux at 2 h was determined to be 2.4%. The surface bound activity remained 15-20% of the total uptake. The radioactivity found in the medium at various time points (observed efflux) appears to be coming mostly from the cell surface bound activity. The internalization and efflux pattern of ⁶⁷Ga-AMBA was found to be the same as that of ¹⁷⁷Lu-AMBA.

Comparison of Internalization and Efflux of Radio-Labeled AMBA by PC-3 Cells

| | % Cell associated activity | | | % Cell associated activity | | | Calc'd |
|---|---|---|---|---|---|---|---|
| | at T = 0 | | | at T = 2 h | | | Efflux |
| Compound | Internalized | Surface | Medium | Internalized | Surface | Medium | T = 2 h |
| ⁶⁷Ga-AMBA | 78.4 (±0.2) | 20.9 (±0.3) | 0.7 (±0.1) | 76.5 (±0.4) | 14.8 (±1.8) | 8.8 (±1.5) | 2.4 (±0.7 |
| ¹⁷⁷Lu-AMBA | 76.8 (±1.8) | 20.5 (±1.6) | 2.3 (±0.2) | 74.7 (±3.0) | 15.3 (±2.3) | 7.3 (±2.2) | 2.9 (±1.8 |

(* Results are the average values of three different experiments)

### Example CIII

### Biodistribution Data of AMBA Labeled with Lutetium-177 or Gallium-67 in PC-3 Tumor-Bearing Immunocompromised Male Mice

Biodistribution studies were performed using trace dose levels of radioactivity (50 μCi/mL) of HPLC purified material. Subjects (n=4/Gallium-67 group, n=9/Lutetium-177 group) were weighed to the nearest 0.1 g, and the weight recorded. Each subject was administered, via i.v. tail vein, a 0.1 mL dose of either ⁶⁷Ga-AMBA (0.0002 μg) or ¹⁷⁷Lu-AMBA (avg. 0.0026 μg). At the end of the residence interval, the subjects were terminated via cervical dislocation. Immediately after termination the designated tissues to be evaluated were harvested. The excised organs, tissues, and blood aliquot were weighed (to the nearest mg) and assayed for residual radioactivity in a Perkin-Elmer, 1480 3″ Wizard automated gamma counter. The material weights and associated counts per minute (cpm) were transferred to an Excel spreadsheet for further statistical analysis using GraphPad™. Lutetium-177 and Gallium-67 exhibited similar overall biodistribution and uptake in target and non-target tissues, with the exception of an increased level in the blood (p≦0.05) using ⁶⁷Ga-AMBA by Student's t-test. In particular, the tumor uptake is comparable at both the 1 and 24 h time points.

### Distribution of Lu-AMBA and Ga-AMBA in a PC-3 Xenograft Mouse Model.

| | ¹⁷⁷Lu-AMBA | ⁶⁷Ga-AMBA | ¹⁷⁷Lu-AMBA | ⁶⁷Ga-AMBA |
|---|---|---|---|---|
| | Mass dose | Mass dose = | Mass dose | Mass dose = |
| | ~0.0026 μg | 0.0002 μg | ~0.0026 μg | 0.0002 μg |
| | n = 9 | n = 4 | n = 9 | n = 4 |
| | 1 h | | 24 h | |
| Tumor (% ID/g) | 6.35 ± 2.23 | 3.90 ± 0.48 | 3.39 ± 0.85 | 2.50 ± 0.27 |
| Blood (% ID) | 0.24 ± 0.09 | 0.49 ± 0.11* | 0.02 ± 0.01 | 0.09 ± 0.02* |
| Liver (% ID) | 0.25 ± 0.08 | 0.35 ± 0.07 | 0.21 ± 0.36 | 0.13 ± 0.03 |
| Kidneys (% ID) | 2.95 ± 0.79 | 2.56 ± 0.48 | 0.91 ± 0.25 | 0.60 ± 0.10 |
| Pancreas (% ID) | 17.78 ± 4.07 | 16.60 ± 1.58 | 12.28 ± 3.50 | 9.94 ± 3.15 |
| GI (% ID) | 11.22 ± 3.29 | 12.17 ± 0.56 | 5.77 ± 1.79 | 4.23 ± 0.46 |
| Skin (% ID/g) | 0.33 ± 0.13 | 0.34 ± 0.07 | 0.10 ± 0.02 | 0.09 ± 0.03 |
| Muscle (% ID/g) | 0.09 ± 0.02 | 0.12 ± 0.08 | 0.03 ± 0.02 | 0.03 ± 0.00 |
| Bladder/Urine (% ID) | 55.66 ± 7.28 | 52.16 ± 7.65 | NA | NA |

*p ≦ 0.05

Based on these data and the in vitro data, Lu-AMBA is a reasonable surrogate to predict the behavior of Ga-AMBA in vitro and in vivo.

### Example CIV

### Detection of the Effect of Tamoxifen on GRP Receptor Binding of BT-474 Human Breast Cancer Cells in a Mouse Xenograft Model

The use of ¹⁷⁷Lu-AMBA as a validated surrogate for ⁶⁷Ga-AMBA had been established previously (Examples XCVI and XCVII), and the surrogate was subsequently used primarily for expediency.

Female nude mice ([Ncr]-Foxn1<nu>) received hormone supplementation by administration of 17β-estradiol via 60 day time released pellets implanted s.c. (0.72 mg/60 Day release; 0.6 mg/kg; Innovative Research of America, Sarasota, Fla.). Four to seven days after beginning hormone treatment, the mice were xenografted with ten million BT-474 cells in Matrigel (1:1 with PBS) s.c. to the flank. The 1713 estradiol is required for tumor cell growth in this model.

Tamoxifen citrate was administered daily to BT-474 tumor-bearing mice once the tumors reached an average size between 100-200 mm³ via time release pellets implanted s.c. (10 mg/21 Day release; 25 mg/kg; Innovative Research of America, Sarasota, Fla.). Control mice received vehicle-only time release pellets (in addition to hormone support).

On day 14 following the start of chemotherapy administration, a low dose pharmacokinetic study was performed using ¹⁷⁷Lu-AMBA as a surrogate for Ga-AMBA to determine the uptake by GRP-R relative to control. In all studies, mice were administered 100 μL of ¹⁷⁷Lu-AMBA at 200 μCi/kg, i.v., with a residence time of 1 h per group (n=3). Tissues were analyzed in an LKB 1282 CompuGamma counter with appropriate standards.

Results: Treatment with Tamoxifen citrate (25 mg/kg) in 132-estradiol supplemented (0.6 mg/kg) BT-474 tumor bearing mice decreased tumor GRP receptor specific signal significantly (P<0.05) as indicated by a decrease in ¹⁷⁷Lu-AMBA binding by 68% over control tumor values. This reduction in uptake in the BT-474 tumors is comparable to that seen with in vitro studies with the same cells on treatment with 4-OH Tamoxifen.

Effect of Tamoxifen on the Distribution of ¹⁷⁷Lu-AMBA at 1 h in BT-474 Tumor-Bearing Nude Mice (Obligate Hormone Supplementation with 17J3-estradiol).

| | Control | + Tamoxifen/+ ES |
|---|---|---|
| BT-474 mice | (n = 2) | (n = 3) |
| Tissue | 1 hr | 1 hr |
| Tumor | 2.18 ± 0.63 | 0.70 ± 0.39* |
| (% ID/g) | | |
| Blood | 0.14 ± 0.06 | 0.13 ± 0.05 |
| (% ID) | | |
| Kidneys | 2.69 ± 0.28 | 2.30 ± 0.88 |
| (% ID) | | |
| Pancreas | 12.50 ± 0.67 | 9.81 ± 2.83 |
| (% ID) | | |
| Gastrointestinal | 11.93 ± 0.43 | 10.93 ± 3.19 |
| (% ID) | | |
| Bladder/urine | 42.79 ± 1.58 | 54.82 ± 6.54 |
| (% ID) | | |

*Significance P < 0.05

### Example CV

### Preparation of ⁶⁸Ga-AMBA

a) Effect of Selenomethionine on ⁶⁸Ga-AMBA RCP: Two sets of experiments in triplicate were done to test the effect of Seleno-L-methionine (Se-Met) on the radiochemical purity (RCP) of ⁶⁸Ga-AMBA. Selenomethionine is an amino acid that has been observed to provide protection against radiolytic damage to sensitive residues in radiolabeled compounds, in particular, the methionine residue in the targeting group of the AMBA ligand (-QWAVGHLM-NH₂).

In the first set of experiments, ⁶⁸Ga-AMBA was prepared in presence of Se-Met, while in the second set, it was prepared without Se-Met. The results of the two sets were compared. To limit exposure to the operator, the syntheses were both performed using the automatic synthesizer TRACERlab FX-FN. In both cases, the ⁶⁸Ga radioisotope solution was obtained by elution of a ⁶⁸Ge/⁶⁸Ga generator, eluted with 5 mL of 0.1 M HCl at the flow rate of 1.5 mL/min using a syringe pump. The peak fraction (1 mL) was collected and used for the study.

Synthesis of ⁶⁸Ga-AMBA in the presence of Se-Met: A formulation solution was prepared by dissolving AMBA ligand (122 to 123 μg) in 0.2 M sodium acetate (NaOAc) buffer, pH 4.5 containing 1 mg/mL Se-Met (Aldrich). The final AMBA concentration was 120 μg/mL. To 1 mL (6.4 to 7.6 mCi) of ⁶⁸Ga generator eluate was added 100 μL of the AMBA formulation (containing 12 μg of AMBA) and 200 μL of a NaOAc solution prepared by mixing 120 μL of H₂O with 80 μL of USP Sodium Acetate for Injection (Hospira), containing 13.1 mg of NaOAc. This solution was heated at 95° C. for 7 minutes, cooled to 30° C. and transferred to a vial for HPLC analysis. To wash the reactor and the tubes from the reactor to the product vial, 10% EtOH in H₂O (3 mL) was introduced into the reactor and transferred to the product vial.

HPLC analysis was performed on a Zorbax Bonus-RP column (4.6×250 mm; 5 μm, Agilent) using a quaternary gradient where A: H₂O; B: 30 mM (NH₄)₂SO₄/0.1% TFA (v: v); C: Acetonitrile (ACN); D: Methanol (MeOH), flow rate=1.5 mL/min, column temp=37° C. The gradient used is shown below.

Mobile Phases
and
Percentage (%)

| Time (min) | A | B | C | D |
|---|---|---|---|---|
| 0 | 30 | 60 | 5 | 5 |
| 5 | 14 | 60 | 13 | 13 |
| 37 | 14 | 60 | 13 | 13 |
| 40 | 0 | 60 | 20 | 20 |
| 45 | 0 | 60 | 20 | 20 |
| 46 | 30 | 60 | 5 | 5 |

Synthesis of 68Ga-AMBA in absence of Se-Met: A formulation solution was prepared by dissolving AMBA ligand (106 to 134 μg) in 0.2 M NaOAc buffer (pH 4.5) to a final concentration of 120 μg/mL. To 1 mL (6.6 to 7.5 mCi) of ⁶⁸Ga generator eluate was added 100 μL of the AMBA formulation (containing 12 μg of AMBA) and 200 μL of a NaOAc solution prepared by mixing 120 μL of H₂O with 80 μL of USP Sodium Acetate for Injection (Hospira), containing 13.1 mg of NaOAc. This solution was heated at 95° C. for 7 minutes, cooled to 30° C. and transferred to a vial for HPLC analysis. To wash the reactor and the tubes from the reactor to the product vial, 10% EtOH in H₂O (3 mL) was introduced into the reactor and transferred to the product vial.

HPLC analysis was performed with the column and the method previously described. The results of the experiments are shown in the following tables 27 and 28 and radiochromatograms. Radiochemical purity (RCP) values obtained at t=0, 1 h, and 2 h post labeling are shown.

TABLE 27
Effect of Selenomethionine on the RCP of ⁶⁸Ga-AMBA
With Se-Met

n = 3

| | t = 0 | t = 1 h | t = 2 h |
|---|---|---|---|
| RCP | 93.7 ± 3.2 | 93.3 ± 2.9 | 92.5 ± 3.5 |
| ⁶⁸Ga-AMBA(Met═O) | 0.6 ± 0.1 | 0.6 ± 0.1 | 0.6 ± 0.1 |
| Free ⁶⁸Ga | 4.0 ± 4 | 4.0 ± 4 | 4.0 ± 4.2 |
| Hydrophilic | 2.2 ± 0.8 | 2.6 ± 1.1 | 3.4 ± 0.8 |
| impurities other than | | | |
| free ⁶⁸Ga | | | |

TABLE 28
Without Se-Met

n = 3

| | t = 0 | t = 1 h | t = 2 h |
|---|---|---|---|
| RCP | 83.3 ± 2.1 | 82.2 ± 2.1 | 81.5 ± 2.2 |
| ⁶⁸Ga-AMBA(Met═O) | 5.5 ± 1 | 5.8 ± 1 | 6.0 ± 1.1 |
| Free ⁶⁸Ga | 2.1 ± 1 | 2.0 ± 0.9 | 1.7 ± 0.6 |
| Hydrophilic | 14.5 ± 3.1 | 15.8 ± 3 | 16.6 ± 2.8 |
| impurities other than | | | |
| free ⁶⁸Ga | | | |

### Typical Radiotrace of ⁶⁸Ga-AMBA Reaction in the Absence (Top Panel) and Presence (Bottom panel) of Selenomethionine

From the previous tables and radiochromatograms it is clear that Se-Met prevented the oxidation of the methionine residue in ⁶⁸Ga-AMBA. It also prevented the formation of other ⁶⁸Ga-labeled hydrophilic impurities. RCP values of up to 96% (93. 7±3.2%) were obtained when Se-Met was used, vs 83.3±2.1% RCP in the absence of Se-Met.

(b) Effect of ascorbic acid and saline on ⁶⁸Ga-AMBA stability. Two sets of experiments in triplicate were done to test the effect of dilution of Sep-Pak purified ⁶⁸Ga-AMBA with a radiostabilizer solution containing ascorbic acid and saline. The stabilizer solution was prepared by mixing 9 parts of normal saline with 1 part ASCOR L 500®. Ascor L 500 (McGuff) is a USP solution of Ascorbic Acid for Injection, and contains 500 mg/mL Sodium Ascorbate, Edetate Disodium (0.025%), Water for Injection (q.s.) and Sodium Bicarbonate for pH adjustment (pH 5.5 to 7.0).

In the first set of experiments ⁶⁸Ga-AMBA, after Sep-Pak purification was mixed with 4 mL of saline, while in the second set, it was diluted with 3 mL of 9 Saline: 1 Ascor and 1 mL of H₂O. The results of the two sets were compared.

### Synthesis of ⁶⁸Ga-AMBA without 9 Saline

### 1 Ascor

To limit exposure to the operator the syntheses were performed using the automatic synthesizer TRACERlab FX-FN. A formulation solution was prepared by dissolving AMBA ligand (120 μg) in 0.2 M sodium acetate (NaOAc) buffer, pH 4.5 containing 1 mg/mL Se-Met (Aldrich). The final AMBA concentration was 120 μg/mL. To 1 mL of ⁶⁸Ga generator eluate was added 100 μL (12 μg) of AMBA formulation and 200 μL of a NaOAc solution prepared mixing 120 μL of H₂O with 80 μL (13.1 mg) of USP NaOAc (Hospira). This solution was heated at 95° C. for 7 minutes, cooled to 30° C. and transferred to a Water C18 Light Sep-Pak conditioned with 3 mL of EtOH and 8 mL of H₂O. To increase recovery, the reactor and the tubing from the reactor to the product vial were washed by introducing 2 ml of 10% EtOH in H₂O into the reactor and transferring this solution to the Sep-Pak. The Sep-Pak was washed with 4 mL of H₂O and the product (5.5 to 5.8 mCi) was eluted into the product vial with 500 μL of EtOH. The Sep-Pak was then rinsed with 4 mL of Saline that were collected in the product vial. HPLC analysis was performed using the column and the method previously described.

Synthesis of ⁶⁸Ga-AMBA with 9 Saline: 1 Ascor.

To 1 mL of ⁶⁸Ga generator eluate was added 100 μL (12 μg) of AMBA formulation prepared as described above and 200 μL of a NaOAc solution prepared mixing 120 μL of H₂O with 80 μL (13.1 mg) of USP NaOAc (Hospira). This solution was heated at 95° C. for 7 minutes, cooled to 30° C. and transferred to a Water C18 light Sep-Pak. To increase recovery the reactor and the tubes from the reactor to the product vial were washed introducing 2 mL of 10% EtOH in H₂O in the reactor and transferring this solution to the Sep-Pak. The Sep-Pak was washed with 4 mL of H₂O and the product (4.6 to 4.9 mCi) was eluted with 500 uL of EtOH into the product vial, which contained 3 mL of a 9:1 mixture of Saline (USP from Hospira): Ascor (McGuff Pharmaceuticals). The Sep-Pak was then rinsed with 1 mL of H₂O that was collected in the product vial.

HPLC analysis was performed with the column and method previously described. The results of the experiments are shown in the following tables 29 and 30:

TABLE 29
+9 Saline: 1 Ascor

| | t = 0 | t = 1 h | t = 2 h |
|---|---|---|---|
| RCP | 97.3 ± 0.1 | 97.3 ± 0.1 | 97.1 ± 0.3 |
| ⁶⁸Ga-AMBA(Met═O) | 0.7 ± 0.1 | 0.7 ± 0.1 | 0.8 ± 0.1 |
| Free ⁶⁸Ga | 0 | 0 | 0 |
| Hydrophilic impurities | 1.6 ± 0.1 | 1.7 ± 0.1 | 1.9 ± 0.3 |
| other than free ⁶⁸Ga | | | |

TABLE 30
−9 Saline: 1 Ascor

| | t = 0 | t = 1 h | t = 2 h |
|---|---|---|---|
| RCP | 97.3 ± 0.4 | 96.6 ± 0.6 | 95.2 ± 0.7 |
| ⁶⁸Ga-AMBA(Met═O) | 0.8 ± 0 | 1.1 ± 0.1 | 1.1 ± 0.2 |
| Free ⁶⁸Ga | 0 | 0 | 0 |
| Hydrophilic | 1.7 ± 0.3 | 2.4 ± 0.6 | 3.8 ± 0.8 |
| impurities other than | | | |
| free ⁶⁸Ga | | | |

The RCP at t=0 and t=1 h are not statistically different (p<0.05). The RCP values for products at t=2 h are statistically different (p≦0.05) indicating that the mixture of 9 parts Saline: 1 part Ascor acts as radioprotectant.

Synthesis of ⁶⁸Ga-AMBA with 1 mL of Generator Eluate and 100 μL of AMBA Formulation

To limit exposure to the operator the syntheses were performed using the automatic synthesizer TRACERlab FX-FN. To 1 mL of ⁶⁸Ga generator eluate was added 100 μL (12 μg) of AMBA formulation prepared as described above and 200 μL of a NaOAc solution prepared mixing 120 μL of H₂O with 80 μL (13.1 mg) of USP NaOAc (Hospira). This solution was heated at 95° C. for 7 minutes, cooled to 30° C. and transferred to a Water C18 light Sep-Pak. To increase recovery, the reactor and the tubes from the reactor to the product vial were washed, introducing 2 mL of 10% EtOH in H₂O to the reactor and transferring this solution to the Sep-Pak. The Sep-Pak was washed with 4 mL of H₂O and the product (4.6 to 4.9 mCi) was eluted into the product vial, containing 3 mL of 9 Saline (USP from Hospira): 1 Ascor (McGuff Pharmaceuticals) with 500 uL of EtOH. The Sep-Pak was then rinsed with 1 mL of H₂O, which was collected into the product vial.

HPLC analysis was performed with the column and method previously described. The results of the experiments are shown in Table 31:

TABLE 31
+9 Saline: 1 Ascor

| | t = 0 | t = 1 h | t = 2 h |
|---|---|---|---|
| RCP | 97.3 ± 0.1 | 97.3 ± 0.1 | 97.1 ± 0.3 |
| ⁶⁸Ga-AMBA(Met═O) | 0.7 ± 0.1 | 0.7 ± 0.1 | 0.8 ± 0.1 |
| Free ⁶⁸Ga | 0 | 0 | 0 |
| Hydrophilic impurities | 1.6 ± 0.1 | 1.7 ± 0.1 | 1.9 ± 0.3 |
| other than free ⁶⁸Ga | | | |

Synthesis of 68Ga-AMBA with 3 mL of Generator Eluate, 400 μL of AMBA Formulation and HPLC Purification

In this study, a larger quantity of Ga generator eluant was used, and the amount of AMBA formulation in the reaction mixture was increased. The product was HPLC purified to remove free ligand after reaction. To limit exposure to the operator the syntheses were performed using the automatic synthesizer TRACERlab FX-FN.

To 3 mL of ⁶⁸Ga generator eluate was added 400 μL (48 μg) of AMBA formulation prepared as described above and 220 μL of USP NaOAc (Hospira) containing 36 mg of NaOAc. This solution was heated at 95° C. for 7 minutes, cooled to 30° C. and 881 μL of H₂O and 500 μL of EtOH were added. This solution was purified by HPLC using a MN Nucleosil column (100-7 C18, 20×250 mm) that was eluted with 29% CH₃CN/81% H₂O at mL/min. The fraction containing the product of interest was collected into a vessel containing 10 mL of H₂O. This solution was loaded onto a Waters C18 Sep-Pak conditioned with 3 mL of EtOH and 8 ml of H₂O. The Sep-Pak was washed with 4 mL of H₂O and the product (4.7 to 5.4 mCi, n=4) was eluted into the product vial with 500 μL of EtOH. The Sep-Pak was then rinsed with 3 mL of H₂O, which was collected in the product vial.

HPLC analysis was performed using the column and method previously described. The results of the experiments are shown in Table 32:

TABLE 32
⁶⁸Ga-AMBA HPLC purified

| | t = 0 | t = 1 h | t = 2 h |
|---|---|---|---|
| RCP | 98.1 ± 0.8 | 97 ± 1.2 | 95.5 ± 1.8 |
| ⁶⁸Ga-AMBA(Met═O) | 0.8 ± 0.3 | 1.3 ± 0.4 | 2.4 ± 1.3 |
| Free ⁶⁸Ga | 0 ± 0 | 0 ± 0 | 0 ± 0 |
| Hydrophilic impurities | 1.9 ± 0.7 | 2.7 ± 1.6 | 4.5 ± 1.8 |
| other than free ⁶⁸Ga | | | |

### Embodiments of the Invention

The following is provided to illustrate without limitation the various embodiments of the present invention:

1. A compound of the general formula:

M-N—O—P-G
wherein
   M is an optical label or a metal chelator, optionally complexed with a radionuclide;
   N is 0, an alpha or non-alpha amino acid or other linking group;
   O is an alpha or non-alpha amino acid; and
   P is 0, an alpha or non-alpha amino acid or other linking group,
   and G is a GRP receptor targeting peptide,
wherein at least one of N, O or P is a non-alpha amino acid.

2. The compound of embodiment 1, wherein G is an agonist or a peptide which confers agonist activity.

3. The compound of embodiment 1, wherein the non-alpha amino acid is selected from the group consisting of:
8-amino-3,6-dioxaoctanoic acid;
N-4-aminoethyl-N-1-piperazine-acetic acid; and
   polyethylene glycol derivatives having the formula NH₂—(CH₂CH₂O)ₙ—CH₂CO₂H or NH₂—(CH₂CH₂O)n-CH₂CH₂CO₂H where n=2 to 100.

4. The compound of embodiment 1, wherein the metal chelator is selected from the group consisting of DTPA, DOTA, DO3A, HP-DO3A, EDTA, TETA, EHPG, HBED, NOTA, DOTMA, TETMA, PDTA, TTHA, LICAM, MECAM and CMDOTA.

5. The compound of embodiment 1, wherein the metal chelator is selected from the group consisting of
N,N-dimethylGly-Ser-Cys;
N,N-dimethylGly-Thr-Cys;
N,N-diethylGly-Ser-Cys; and
N,N-dibenzylGly-Ser-Cys.

6. The compound of embodiment 1, wherein the metal chelator is selected from the group consisting of
N,N-dimethylGly-Ser-Cys-Gly;
N,N-dimethylGly-Thr-Cys-Gly;
N,N-diethylGly-Ser-Cys-Gly; and
N,N-dibenzylGly-Ser-Cys-Gly.

7. The compound of embodiment 1, selected from the group consisting of:

methylglycine-Ser-Cys(Acm)-Gly-Lys-8-amino-3,6-dioxaoctanoic acid-BBN(7-14) wherein the BBN(7-14) sequence is SEQ ID NO: 1;
methylglycine-Ser-Cys(Acm)-Gly-Arg-8-amino-3,6-dioxaoctanoic acid-BBN(7-14) wherein the BBN(7-14) sequence is SEQ ID NO: 1;
methylglycine-Ser-Cys(Acm)-Gly-Asp-8-amino-3,6-dioxaoctanoic acid-BBN(7-14) wherein the BBN(7-14) sequence is SEQ ID NO: 1;
methylglycine-Ser-Cys(Acm)-Gly-Ser-8-amino-3,6-dioxaoctanoic acid-BBN(7-14) wherein the BBN(7-14) sequence is SEQ ID NO: 1;
methylglycine-Ser-Cys(Acm)-Gly-Gly-8-amino-3,6-dioxaoctanoic acid-BBN(7-14) wherein the BBN(7-14) sequence is SEQ ID NO: 1;
methylglycine-Ser-Cys(Acm)-Gly-Glu-8-amino-3,6-dioxaoctanoic acid-BBN(7-14) wherein the BBN(7-14) sequence is SEQ ID NO: 1;
methylglycine-Ser-Cys(Acm)-Gly-Dala-8-amino-3,6-dioxaoctanoic acid-BBN(7-14) wherein the BBN(7-14) sequence is SEQ ID NO: 1;
methylglycine-Ser-Cys(Acm)-Gly-8-amino-3,6-dioxaoctanoic acid-Lys-BBN(7-14) wherein the BBN(7-14) sequence is SEQ ID NO: 1;
methylglycine-Ser-Cys(Acm)-Gly-8-amino-3,6-dioxaoctanoic acid-Arg-BBN(7-14) wherein the BBN(7-14) sequence is SEQ ID NO: 1;
methylglycine-Ser-Cys(Acm)-Gly-8-amino-3,6-dioxaoctanoic acid-Asp-BBN(7-14) wherein the BBN(7-14) sequence is SEQ ID NO: 1;
methylglycine-Ser-Cys(Acm)-Gly-8-amino-3,6-dioxaoctanoic acid-Ser-BBN(7-14) wherein the BBN(7-14) sequence is SEQ ID NO: 1;
methylglycine-Ser-Cys(Acm)-Gly-8-amino-3,6-dioxaoctanoic acid-Gly-BBN(7-14) wherein the BBN(7-14) sequence is SEQ ID NO: 1;
methylglycine-Ser-Cys(Acm)-Gly-8-amino-3,6-dioxaoctanoic acid-Glu-BBN(7-14) wherein the BBN(7-14) sequence is SEQ ID NO: 1;
methylglycine-Ser-Cys(Acm)-Gly-8-amino-3,6-dioxaoctanoic acid-Dala-BBN(7-14) wherein the BBN(7-14) sequence is SEQ ID NO: 1;
methylglycine-Ser-Cys(Acm)-Gly-8-amino-3,6-dioxaoctanoic acid-8-amino-3,6-dioxaoctanoic acid-BBN(7-14) wherein the BBN(7-14) sequence is SEQ ID NO: 1;
methylglycine-Ser-Cys(Acm)-Gly-8-amino-3,6-dioxaoctanoic acid-2,3-diaminopropionic acid-BBN(7-14) wherein the BBN(7-14) sequence is SEQ ID NO: 1;
methylglycine-Ser-Cys(Acm)-Gly-8-amino-3,6-dioxaoctanoic acid-BBN(7-14) wherein the BBN(7-14) sequence is SEQ ID NO: 1;
methylglycine-Ser-Cys(Acm)-Gly-2,3-diaminopropionic acid-8-amino-3,6-dioxaoctanoic acid-BBN(7-14) wherein the BBN(7-14) sequence is SEQ ID NO: 1;
methylglycine-Ser-Cys(Acm)-Gly-Asp-8-amino-3,6-dioxaoctanoic acid-Gly-BBN(7-14) wherein the BBN(7-14) sequence is SEQ ID NO: 1;
methylglycine-Ser-Cys(Acm)-Gly-8-amino-3,6-dioxaoctanoic acid-Asp-Gly-BBN(7-14) wherein the BBN(7-14) sequence is SEQ ID NO: 1;
methylglycine-Ser-Cys(Acm)-Gly-8-amino-3,6-dioxaoctanoic acid-Ser-Gly-BBN(7-14) wherein the BBN(7-14) sequence is SEQ ID NO: 1;
methylglycine-Ser-Cys(Acm)-Gly-8-amino-3,6-dioxaoctanoic acid-Arg-Gly-BBN(7-14) wherein the BBN(7-14) sequence is SEQ ID NO: 1;
methylglycine-Ser-Cys(Acm)-Gly-8-amino-3,6-dioxaoctanoic acid-8-amino-3,6-dioxaoctanoic acid-Gly-BBN(7-14) wherein the BBN(7-14) sequence is SEQ ID NO: 1;
methylglycine-Ser-Cys(Acm)-Gly-8-amino-3,6-dioxaoctanoic acid-2,3-diaminopropionic acid-Gly-BBN(7-14) wherein the BBN(7-14) sequence is SEQ ID NO: 1;
methylglycine-Ser-Cys(Acm)-Gly-8-amino-3,6-dioxaoctanoic acid-Lys-Gly-BBN(7-14) wherein the BBN(7-14) sequence is SEQ ID NO: 1;
methylglycine-Ser-Cys(Acm)-Gly-2,3-diaminopropionic acid-8-amino-3,6-dioxaoctanoic acid-Gly-BBN(7-14) wherein the BBN(7-14) sequence is SEQ ID NO: 1;
methylglycine-Ser-Cys(Acm)-Gly-N-4-aminoethyl-N-1-piperazineacetic acid-Asp-BBN(7-14) wherein the BBN(7-14) sequence is SEQ ID NO: 1;
methylglycine-Ser-Cys(Acm)-Gly-N-4-aminoethyl-N-1-piperazineacetic acid-Ser-BBN(7-14) wherein the BBN(7-14) sequence is SEQ ID NO: 1;
methylglycine-Ser-Cys(Acm)-Gly-N-4-aminoethyl-N-1-piperazineacetic acid-Arg-BBN(7-14) wherein the BBN(7-14) sequence is SEQ ID NO: 1;
methylglycine-Ser-Cys(Acm)-Gly-N-4-aminoethyl-N-1-piperazineacetic acid-8-amino-3,6-dioxaoctanoic acid-BBN(7-14) wherein the BBN(7-14) sequence is SEQ ID NO: 1;
methylglycine-Ser-Cys(Acm)-Gly-N-4-aminoethyl-N-1-piperazineacetic acid-2,3-diaminopropionic acid-BBN(7-14) wherein the BBN(7-14) sequence is SEQ ID NO: 1;
methylglycine-Ser-Cys(Acm)-Gly-N-4-aminoethyl-N-1-piperazineacetic acid-Lys-BBN(7-14) wherein the BBN(7-14) sequence is SEQ ID NO: 1;
methylglycine-Ser-Cys(Acm)-Gly-N-1-piperazineacetic acid-Asp-BBN(7-14) wherein the BBN(7-14) sequence is SEQ ID NO: 1;
methylglycine-Ser-Cys(Acm)-Gly-N-1-piperazineacetic acid-Ser-BBN(7-14) wherein the BBN(7-14) sequence is SEQ ID NO: 1;
methylglycine-Ser-Cys(Acm)-Gly-N-1-piperazineacetic acid-Arg-BBN(7-14) wherein the BBN(7-14) sequence is SEQ ID NO: 1;
methylglycine-Ser-Cys(Acm)-Gly-N-1-piperazineacetic acid-8-amino-3,6-dioxaoctanoic acid-BBN(7-14) wherein the BBN(7-14) sequence is SEQ ID NO: 1;
methylglycine-Ser-Cys(Acm)-Gly-N-1-piperazineacetic acid-2,3-diaminopropionic acid BBN(7-14) wherein the BBN(7-14) sequence is SEQ ID NO: 1;
methylglycine-Ser-Cys(Acm)-Gly-N-1-piperazineacetic acid-Lys-BBN(7-14) wherein the BBN(7-14) sequence is SEQ ID NO: 1;
methylglycine-Ser-Cys(Acm)-Gly-4-Hydroxyproline-8-amino-3,6-dioxaoctanoic acid-BBN(7-14) wherein the BBN(7-14) sequence is SEQ ID NO: 1;
methylglycine-Ser-Cys(Acm)-Gly-4-aminoproline-8-amino-3,6-dioxaoctanoic acid-BBN(7-14) wherein the BBN(7-14) sequence is SEQ ID NO: 1;
methylglycine-Ser-Cys(Acm)-Gly-Lys-8-amino-3,6-dioxaoctanoic acid-Gly-BBN(7-14) wherein the BBN(7-14) sequence is SEQ ID NO: 1;
methylglycine-Ser-Cys(Acm)-Gly-Arg-8-amino-3,6-dioxaoctanoic acid-Gly-BBN(7-14) wherein the BBN(7-14) sequence is SEQ ID NO: 1;
methylglycine-Ser-Cys(Acm)-Gly-Ser-8-amino-3,6-dioxaoctanoic acid-Gly-BBN(7-14) wherein the BBN(7-14) sequence is SEQ ID NO: 1;
methylglycine-Ser-Cys(Acm)-Gly-Asp-8-amino-3,6-dioxaoctanoic acid-Gly-BBN(7-14) wherein the BBN(7-14) sequence is SEQ ID NO: 1;
methylglycine-Ser-Cys(Acm)-Gly-8-amino-3,6-dioxaoctanoic acid-Asp-Gly-BBN(7-14) wherein the BBN(7-14) sequence is SEQ ID NO: 1;
methylglycine-Ser-Cys(Acm)-Gly-8-amino-3,6-dioxaoctanoic acid-Ser-Gly-BBN(7-14) wherein the BBN(7-14) sequence is SEQ ID NO: 1;
methylglycine-Ser-Cys(Acm)-Gly-8-amino-3,6-dioxaoctanoic acid-Arg-Gly-BBN(7-14) wherein the BBN(7-14) sequence is SEQ ID NO: 1;
methylglycine-Ser-Cys(Acm)-Gly-8-amino-3,6-dioxaoctanoic acid-8-amino-3,6-dioxaoctanoic acid-Gly-BBN(7-14) wherein the BBN(7-14) sequence is SEQ ID NO: 1;
methylglycine-Ser-Cys(Acm)-Gly-8-amino-3,6-dioxaoctanoic acid-2,3-diaminopropionic acid-Gly-BBN(7-14) wherein the BBN(7-14) sequence is SEQ ID NO: 1;
methylglycine-Ser-Cys(Acm)-Gly-8-amino-3,6-dioxaoctanoic acid-Lys-Gly-BBN(7-14) wherein the BBN(7-14) sequence is SEQ ID NO: 1; and
methylglycine-Ser-Cys(Acm)-Gly-2,3-diaminopropionic acid-8-amino-3,6-dioxaoctanoic acid-Gly-BBN(7-14) wherein the BBN(7-14) sequence is SEQ ID NO: 1.

8. The compound of embodiment 1, selected from the group consisting of:

methylglycine-Ser-Cys-Gly-Lys-8-amino-3,6-dioxaoctanoic acid-BBN(7-14) wherein the BBN(7-14) sequence is SEQ ID NO: 1;
methylglycine-Ser-Cys-Gly-Arg-8-amino-3,6-dioxaoctanoic acid-BBN(7-14) wherein the BBN(7-14) sequence is SEQ ID NO: 1;
methylglycine-Ser-Cys-Gly-Asp-8-amino-3,6-dioxaoctanoic acid-BBN(7-14) wherein the BBN(7-14) sequence is SEQ ID NO: 1;
methylglycine-Ser-Cys-Gly-Ser-8-amino-3,6-dioxaoctanoic acid-BBN(7-14) wherein the BBN(7-14) sequence is SEQ ID NO: 1;
methylglycine-Ser-Cys-Gly-Gly-8-amino-3,6-dioxaoctanoic acid-BBN(7-14) wherein the BBN(7-14) sequence is SEQ ID NO: 1;
methylglycine-Ser-Cys-Gly-Glu-8-amino-3,6-dioxaoctanoic acid-BBN(7-14) wherein the BBN(7-14) sequence is SEQ ID NO: 1;
methylglycine-Ser-Cys-Gly-Dala-8-amino-3,6-dioxaoctanoic acid-BBN(7-14) wherein the BBN(7-14) sequence is SEQ ID NO: 1;
methylglycine-Ser-Cys-Gly-8-amino-3,6-dioxaoctanoic acid-Lys-BBN(7-14) wherein the BBN(7-14) sequence is SEQ ID NO: 1;
methylglycine-Ser-Cys-Gly-8-amino-3,6-dioxaoctanoic acid-Arg-BBN(7-14) wherein the BBN(7-14) sequence is SEQ ID NO: 1;
methylglycine-Ser-Cys-Gly-8-amino-3,6-dioxaoctanoic acid-Asp-BBN(7-14) wherein the BBN(7-14) sequence is SEQ ID NO: 1;
methylglycine-Ser-Cys-Gly-8-amino-3,6-dioxaoctanoic acid-Ser-BBN(7-14) wherein the BBN(7-14) sequence is SEQ ID NO: 1;
methylglycine-Ser-Cys-Gly-8-amino-3,6-dioxaoctanoic acid-Gly-BBN(7-14) wherein the BBN(7-14) sequence is SEQ ID NO: 1;
methylglycine-Ser-Cys-Gly-8-amino-3,6-dioxaoctanoic acid-Glu-BBN(7-14) wherein the BBN(7-14) sequence is SEQ ID NO: 1;
methylglycine-Ser-Cys-Gly-8-amino-3,6-dioxaoctanoic acid-Dala-BBN(7-14) wherein the BBN(7-14) sequence is SEQ ID NO: 1;
methylglycine-Ser-Cys-Gly-8-amino-3,6-dioxaoctanoic acid-8-amino-3,6-dioxaoctanoic acid-BBN(7-14) wherein the BBN(7-14) sequence is SEQ ID NO: 1;
methylglycine-Ser-Cys-Gly-8-amino-3,6-dioxaoctanoic acid-2,3-diaminopropionic acid-BBN(7-14) wherein the BBN(7-14) sequence is SEQ ID NO: 1;
methylglycine-Ser-Cys-Gly-8-amino-3,6-dioxaoctanoic acid-BBN(7-14) wherein the BBN(7-14) sequence is SEQ ID NO: 1;
methylglycine-Ser-Cys-Gly-2,3-diaminopropionic acid-8-amino-3,6-dioxaoctanoic acid-BBN(7-14) wherein the BBN(7-14) sequence is SEQ ID NO: 1;
methylglycine-Ser-Cys-Gly-Asp-8-amino-3,6-dioxaoctanoic acid-Gly-BBN(7-14) wherein the BBN(7-14) sequence is SEQ ID NO: 1;
methylglycine-Ser-Cys-Gly-8-amino-3,6-dioxaoctanoic acid-Asp-Gly-BBN(7-14) wherein the BBN(7-14) sequence is SEQ ID NO: 1;
methylglycine-Ser-Cys-Gly-8-amino-3,6-dioxaoctanoic acid-Ser-Gly-BBN(7-14) wherein the BBN(7-14) sequence is SEQ ID NO: 1;
methylglycine-Ser-Cys-Gly-8-amino-3,6-dioxaoctanoic acid-Arg-Gly-BBN(7-14) wherein the BBN(7-14) sequence is SEQ ID NO: 1;
methylglycine-Ser-Cys-Gly-8-amino-3,6-dioxaoctanoic acid-8-amino-3,6-dioxaoctanoic acid-Gly-BBN(7-14) wherein the BBN(7-14) sequence is SEQ ID NO: 1;
methylglycine-Ser-Cys-Gly-8-amino-3,6-dioxaoctanoic acid-2,3-diaminopropionic acid-Gly-BBN(7-14) wherein the BBN(7-14) sequence is SEQ ID NO: 1;
methylglycine-Ser-Cys-Gly-8-amino-3,6-dioxaoctanoic acid-Lys-Gly-BBN(7-14) wherein the BBN(7-14) sequence is SEQ ID NO: 1;
methylglycine-Ser-Cys-Gly-2,3-diaminopropionic acid-8-amino-3,6-dioxaoctanoic acid-Gly-BBN(7-14) wherein the BBN(7-14) sequence is SEQ ID NO: 1;
methylglycine-Ser-Cys-Gly-N-4-aminoethyl-N-1-piperazineacetic acid-Asp-BBN(7-14) wherein the BBN(7-14) sequence is SEQ ID NO: 1;
methylglycine-Ser-Cys-Gly-N-4-aminoethyl-N-1-piperazineacetic acid-Ser-BBN(7-14) wherein the BBN(7-14) sequence is SEQ ID NO: 1;
methylglycine-Ser-Cys-Gly-N-4-aminoethyl-N-1-piperazineacetic acid-Arg-BBN(7-14) wherein the BBN(7-14) sequence is SEQ ID NO: 1;
methylglycine-Ser-Cys(Acm)-Gly-N-4-aminoethyl-N-1-piperazineacetic acid-8-amino-3,6-dioxaoctanoic acid-BBN(7-14) wherein the BBN(7-14) sequence is SEQ ID NO: 1;
methylglycine-Ser-Cys-Gly-N-4-aminoethyl-N-1-piperazineacetic acid-2,3-diaminopropionic acid-BBN(7-14) wherein the BBN(7-14) sequence is SEQ ID NO: 1;
methylglycine-Ser-Cys-Gly-N-4-aminoethyl-N-1-piperazineacetic acid-Lys-BBN(7-14) wherein the BBN(7-14) sequence is SEQ ID NO: 1;
methylglycine-Ser-Cys-Gly-N-1-piperazineacetic acid-Asp-BBN(7-14) wherein the BBN(7-14) sequence is SEQ ID NO: 1;
methylglycine-Ser-Cys-Gly-N-1-piperazineacetic acid-Ser-BBN(7-14) wherein the BBN(7-14) sequence is SEQ ID NO: 1;
methylglycine-Ser-Cys-Gly-N-1-piperazineacetic acid-Arg-BBN(7-14) wherein the BBN(7-14) sequence is SEQ ID NO: 1;
methylglycine-Ser-Cys-Gly-N-1-piperazineacetic acid-8-amino-3,6-dioxaoctanoic acid-BBN(7-14) wherein the BBN(7-14) sequence is SEQ ID NO: 1;
methylglycine-Ser-Cys-Gly-N-1-piperazineacetic acid-2,3-diaminopropionic acid BBN(7-14) wherein the BBN(7-14) sequence is SEQ ID NO: 1;
methylglycine-Ser-Cys-Gly-N-1-piperazineacetic acid-Lys-BBN(7-14) wherein the BBN(7-14) sequence is SEQ ID NO: 1;
methylglycine-Ser-Cys-Gly-4-Hydroxyproline-8-amino-3,6-dioxaoctanoic acid-BBN(7-14) wherein the BBN(7-14) sequence is SEQ ID NO: 1;
methylglycine-Ser-Cys-Gly-4-aminoproline-8-amino-3,6-dioxaoctanoic acid-BBN(7-14) wherein the BBN(7-14) sequence is SEQ ID NO: 1;
methylglycine-Ser-Cys-Gly-Lys-8-amino-3,6-dioxaoctanoic acid-Gly-BBN(7-14) wherein the BBN(7-14) sequence is SEQ ID NO: 1;
methylglycine-Ser-Cys-Gly-Arg-8-amino-3,6-dioxaoctanoic acid-Gly-BBN(7-14) wherein the BBN(7-14) sequence is SEQ ID NO: 1;
methylglycine-Ser-Cys-Gly-Ser-8-amino-3,6-dioxaoctanoic acid-Gly-BBN(7-14) wherein the BBN(7-14) sequence is SEQ ID NO: 1;
methylglycine-Ser-Cys-Gly-Asp-8-amino-3,6-dioxaoctanoic acid-Gly-BBN(7-14) wherein the BBN(7-14) sequence is SEQ ID NO: 1;
methylglycine-Ser-Cys-Gly-8-amino-3,6-dioxaoctanoic acid-Asp-Gly-BBN(7-14) wherein the BBN(7-14) sequence is SEQ ID NO: 1;
methylglycine-Ser-Cys-Gly-8-amino-3,6-dioxaoctanoic acid-Ser-Gly-BBN(7-14) wherein the BBN(7-14) sequence is SEQ ID NO: 1;
methylglycine-Ser-Cys-Gly-8-amino-3,6-dioxaoctanoic acid-Arg-Gly-BBN(7-14) wherein the BBN(7-14) sequence is SEQ ID NO: 1;
methylglycine-Ser-Cys-Gly-8-amino-3,6-dioxaoctanoic acid-8-amino-3,6-dioxaoctanoic acid-Gly-BBN(7-14) wherein the BBN(7-14) sequence is SEQ ID NO: 1;
methylglycine-Ser-Cys-Gly-8-amino-3,6-dioxaoctanoic acid-2,3-diaminopropionic acid-Gly-BBN(7-14) wherein the BBN(7-14) sequence is SEQ ID NO: 1;
methylglycine-Ser-Cys-Gly-8-amino-3,6-dioxaoctanoic acid-Lys-Gly-BBN(7-14) wherein the BBN(7-14) sequence is SEQ ID NO: 1; and
methylglycine-Ser-Cys-Gly-2,3-diaminopropionic acid-8-amino-3,6-dioxaoctanoic acid-Gly-BBN(7-14) wherein the BBN(7-14) sequence is SEQ ID NO: 1.

9. The compound of embodiment 1, selected from the group consisting of:

-monoamide-8-amino-3,6-dioxaoctanoic acid-diaminopropionic acid-BBN(7-14) wherein the BBN(7-14) sequence is SEQ ID NO: 1;
-monoamide-8-amino-3,6-dioxaoctanoic acid-biphenylalanine-BBN(7-14) wherein the BBN(7-14) sequence is SEQ ID NO: 1;
-monoamide-8-amino-3,6-dioxaoctanoic acid-diphenylalanine-BBN(7-14) wherein the BBN(7-14) sequence is SEQ ID NO: 1;
-monoamide-8-amino-3,6-dioxaoctanoic acid-4-benzoylphenylalanine-BBN(7-14) wherein the BBN(7-14) sequence is SEQ ID NO: 1;
-monoamide-5-aminopentanoic acid-8-amino-3,6-dioxaoctanoic acid-BBN(7-14) wherein the BBN(7-14) sequence is SEQ ID NO: 1;
-monoamide-8-amino-3,6-dioxaoctanoic acid-D-phenylalanine-BBN(7-14) wherein the BBN(7-14) sequence is SEQ ID NO: 1; and
-monoamide-8-aminooctanoic acid-8-amino-3,6-dioxaoctanoic acid-BBN(7-14) wherein the BBN(7-14) sequence is SEQ ID NO: 1.

10. The compound of any one of embodiments 1, 2 or 3, wherein the optical label is selected from the group consisting of organic chromophores, organic fluorophores, light-absorbing compounds, light-reflecting compounds, light-scattering compounds, and bioluminescent molecules.

11. A method of imaging comprising the steps of:
administering to a patient a diagnostic imaging agent comprising the compound of embodiment 1 wherein M is a metal chelator complexed with a diagnostic radionuclide, and
imaging said patient.

12. A method of imaging comprising the steps of:
administering to a patient a diagnostic imaging agent comprising the compound of embodiment 8 complexed with a diagnostic radionuclide, and
imaging said patient.

13. A method of imaging comprising the steps of:
administering to a patient a diagnostic imaging agent comprising the compound of embodiment 1 wherein M is an optical label, and
imaging said patient.

14. A method of imaging comprising the steps of:
administering to a patient a diagnostic imaging agent comprising the compound of embodiment 10, and
imaging said patient.

15. A method for preparing a diagnostic imaging agent comprising the step of adding to an injectable medium a substance comprising the compound of embodiment 1.

16. A method of treating a patient comprising the step of administering to a patient a radiotherapeutic agent comprising the compound of embodiments 7, 8 or 9 complexed with a therapeutic radionuclide.

17. A method of treating a patient comprising the step of administering to a patient a radiotherapeutic agent comprising the compound of embodiment 4 complexed with a therapeutic radionuclide.

18. A method of preparing a radiotherapeutic agent comprising the step of adding to an injectable medium a substance comprising the compound of embodiments 7, 8, or 9.

19. A method of preparing a radiotherapeutic agent comprising the step of adding to an injectable medium a substance comprising the compound of embodiment 4.

20. A compound of the general formula:

M-N—O—P-G
wherein
   M is an optical label or a metal chelator, optionally complexed with a radionuclide;
   N is 0, an alpha amino acid, a substituted bile acid or other linking group;
   O is an alpha amino acid or a substituted bile acid; and
   P is 0, an alpha amino acid, a substituted bile acid or other linking group; and
   G is a GRP receptor targeting peptide, and
wherein at least one of N, O or P is a substituted bile acid.

21. The compound of embodiment 20, wherein G is an agonist or a peptide which confers agonist activity.

22. The compound of embodiment 20, wherein the substituted bile acid is selected from the group consisting of:
3β-amino-3-deoxycholic acid;
(3β,5β)-3-aminocholan-24-oic acid;
(3β,5β,12α)-3-amino-12-hydroxycholan-24-oic acid;
(3β,5β,7α,12α)-3-amino-7,12-dihydroxycholan-24-oic acid;
Lys-(3,6,9)-trioxaundecane-1,1′-dicarbonyl-3,7-dideoxy-3-aminocholic acid);
(3β,5β,7α,12α)-3-amino-7-hydroxy-12-oxocholan-24-oic acid; and
(3β,5β,7α)-3-amino-7-hydroxycholan-24-oic acid.

23. The compound of embodiment 20, wherein M is selected from the group consisting of: DTPA, DOTA, DO3A, HPDO3A, EDTA, TETA and CMDOTA.

24. The compound of embodiment 20, wherein M is selected from the group consisting of EHPG and derivatives thereof.

25. The compound of embodiment 20, wherein M is selected from the group consisting of 5-Cl-EHPG, 5-Br-EHPG, 5-Me-EHPG, 5-t-Bu-EHPG, and 5-sec-Bu-EHPG.

26. The compound of embodiment 20, wherein M is selected from the group consisting of benzodiethylenetriamine pentaacetic acid (benzo-DTPA) and derivatives thereof.

27. The compound of embodiment 20, wherein M is selected from the group consisting of dibenzo-DTPA, phenyl-DTPA, diphenyl-DTPA, benzyl-DTPA, and dibenzyl DTPA.

28. The compound of embodiment 20, wherein M is selected from the group consisting of HBED and derivatives thereof.

29. The compound of embodiment 20, wherein M is a macrocyclic compound which contains at least 3 carbon atoms and at least two heteroatoms (O and/or N), which macrocyclic compounds can consist of one ring, or two or three rings joined together at the hetero ring elements.

30. The compound of embodiment 20, wherein M is selected from the group consisting of benzo-DOTA, dibenzo-DOTA, and benzo-NOTA, benzo-TETA, benzo-DOTMA, and benzo-TETMA.

31. The compound of embodiment 20, wherein M is selected from the group consisting of derivatives of 1,3-propylenediaminetetraacetic acid (PDTA) and triethylenetetraaminehexaacetic acid (TTHA); derivatives of

1,5,10-N,N′,N″-tris(2,3-dihydroxybenzoyl)-tricatecholate (LICAM) and
1,3,5-N,N′,N″-tris(2,3-dihydroxybenzoyl)aminomethylbenzene (MECAM).

32. A compound of embodiment 20 selected from the group consisting of:

-monoamide-Gly-(3β,5β)-3-aminocholan-24-oic acid-BBN(7-14) wherein the BBN(7-14) sequence is SEQ ID NO: 1;
-monoamide-Gly-(3J_{3,5}J_{3,12}a)-3-amino-12-hydroxycholan-24-oic acid-BBN(7-14) wherein the BBN(7-14) sequence is SEQ ID NO: 1;
-monoamide-Gly-(3β,5β,7α,12α)-3-amino-7,12-dihydroxycholan-24-oic acid-BBN(7-14) wherein the BBN(7-14) sequence is SEQ ID NO: 1;
-monoamide-Gly-Lys-(3,6,9)-trioxaundecane-1,1′-dicarbonyl-3,7-dideoxy-3-aminocholic acid)-Arg-BBN(7-14) wherein the BBN(7-14) sequence is SEQ ID NO: 1; (3β,5β,7α,12α)-3-amino-7,12-dihydroxycholan-24-oic acid-3,6,9-trioxaundecane-1,1′-dicarbonyl Lys(DO3A-monoamide-Gly)-Arg-BBN(7-14) wherein the BBN(7-14) sequence is SEQ ID NO: 1;
-monoamide-Gly-(3β,5β,7α,12α)-3-amino-12-oxocholan-24-oic acid-BBN(7-14) wherein the BBN(7-14) sequence is SEQ ID NO: 1;
-monoamide-1-amino-3,6-dioxaoctanoic acid-(3β,5β,7α,12α)-3-amino-7,12-dihydroxycholan-24-oic acid-BBN(7-14) wherein the BBN(7-14) sequence is SEQ ID NO: 1;
-monoamide-Gly-(3β,5β,7α,12α)-3-amino-7,12-dihydroxycholan-24-oic acid-QWAVaHLM-NH₂ wherein QWAVaHLM-NH₂ is SEQ ID NO: 14;
-monoamide-Gly-(3β,5β,7α,12α)-3-amino-7,12-dihydroxycholan-24-oic acid-f-QWAVGHLM-NH wherein QWAVGHLM-NH₂ is SEQ ID NO: 1;
-monoamide-Gly-(3β,5β,7α,12α)-3-amino-7,12-dihydroxycholan-24-oic acid-f-WAVGHLL-NH₂ wherein WAVGHLL-NH₂ is SEQ ID NO: 26;
-monoamide-Gly-(3β,5β,7α,12α)-3-amino-7,12-dihydroxycholan-24-oic acid-f-QWAVGHL-NH-pentyl wherein QWAVGHL-NH-pentyl is SEQ ID NO: 6;
-monoamide-Gly-(3β,5β,7α,12α)-3-amino-7,12-dihydroxycholan-24-oic acid-γ-QWAV-Bala-H-F-Nle-NH₂ wherein QWAV-Bala-H-F-Nle-NH₂ is SEQ ID NO: 9;
-monoamide-Gly-(3β,5β,7α,12α)-3-amino-7,12-dihydroxycholan-24-oic acid-f-QWAV-Bala-H-F-Nle-NH₂ wherein QWAV-Bala-H-F-Nle-NH₂ is SEQ ID NO: 9;
-monoamide-Gly-(3β,5β,7α,12α)-3-amino-7,12-dihydroxycholan-24-oic acid-QWAVGHFL-NH₂ wherein QWAVGHFL-NH₂ is SEQ ID NO: 22
-monoamide-Gly-(3β,5β,7α,12α)-3-amino-7,12-dihydroxycholan-24-oic acid-QWAVGNMeH-L-M-NH₂ wherein QWAVGNMeH-L-M-NH₂ is SEQ ID NO: 15;
-monoamide-Gly-(3β,5β,7α,12α)-3-amino-7,12-dihydroxycholan-24-oic acid-LWAVGSF-M-NH₂ wherein LWAVGSF-M-NH₂ is SEQ ID NO: 11;
-monoamide-Gly-(3β,5β,7α,12α)-3-amino-7,12-dihydroxycholan-24-oic acid-HWAVGHLM-NH₂ wherein HWAVGHLM-NH₂ is SEQ ID NO: 12;
-monoamide-Gly-(3β,5β,7α,12α)-3-amino-7,12-dihydroxycholan-24-oic acid-LWAGHFM-NH₂ wherein LWAGHFM-NH₂ is SEQ ID NO: 20;
-monoamide-Gly-(3β,5β,7α,12α)-3-amino-7,12-dihydroxycholan-24-oic acid-QWAVGHFM-NH₂ wherein QWAVGHFM-NH₂ is SEQ ID NO: 13;
-monoamide-Gly-(3β,5β,7α,12α)-3-amino-7,12-dihydroxycholan-24-oic acid-QRLGNQWAVGHLM-NH₂ wherein QRLGNQWAVGHLM-NH₂ is SEQ ID NO: 3;
-monoamide-Gly-(3β,5β,7α,12α)-3-amino-7,12-dihydroxycholan-24-oic acid-QRYGNQWAVGHLM-NH₂ wherein QRYGNQWAVGHLM-NH₂ is SEQ ID NO: 4;
-monoamide-Gly-(3β,5β,7α,12α)-3-amino-7,12-dihydroxycholan-24-oic acid-QKYGNQWAVGHLM-NH₂ wherein QKYGNQWAVGHLM-NH₂ is SEQ ID NO: 5;
Lys (DO3A-monoamide)-Gly-(3β,5β,7α,12α)-3-amino-7,12-dihydroxycholan-24-oic acid-LGNQWAVGHLM-NH₂ wherein LGNQWAVGHLM-NH₂ is SEQ ID NO: 18.
   -monoamide-Gly-3-amino-3-deoxycholic acid-QRLGNQWAVGHLM-NH₂ wherein QRLGNQWAVGHLM-NH₂ is SEQ ID NO: 3;
   -monoamide-Gly-3-amino-3-deoxycholic acid-QRYGNQWAVGHLM-NH₂ wherein QRYGNQWAVGHLM-NH₂ is SEQ ID NO: 4;
   -monoamide-Gly-3-amino-3-deoxycholic acid-QKYGNQWAVGHLM-NH₂ wherein QKYGNQWAVGHLM-NH₂ is SEQ ID NO: 5; and
Pglu-Q-Lys(DO3A-monoamide-G-3-amino-3-deoxycholic acid)-LGNQWAVGHLM-NH₂ wherein LGNQWAVGHLM-NH₂ is SEQ ID NO: 18.

33. The compound of any one of embodiments 20, 21 or 22, wherein the optical label is selected from the group consisting of organic chromophores, organic fluorophores, light-absorbing compounds, light-reflecting compounds, light-scattering compounds, and bioluminescent molecules.

34. A method of imaging comprising the steps of:
administering to a patient a diagnostic imaging agent comprising the compound of embodiment 20 wherein M is a metal chelator complexed with a diagnostic radionuclide, and
imaging said patient.

35. A method of imaging comprising the steps of:
administering to a patient a diagnostic imaging agent comprising the compound of embodiment 32, and
imaging said patient.

36. A method of imaging comprising the steps of:
administering to a patient a diagnostic imaging agent comprising the compound of embodiment 20 wherein M is an optical label, and
imaging said patient.

37. A method of imaging comprising the steps of:
administering to a patient a diagnostic imaging agent comprising the compound of embodiment 33, and
imaging said patient.

38. A method for preparing a diagnostic imaging agent comprising the step of adding to an injectable medium a substance comprising the compound of embodiment 20.

39. A method of treating a patient comprising the step of administering to a patient a radiotherapeutic agent comprising the compound of embodiment 20 complexed with a therapeutic radionuclide.

40. A method of preparing a radiotherapeutic agent comprising the step of adding to an injectable medium a substance comprising the compound of embodiment 20.

41. A compound DO3A-monoamide-Gly-(3β,5β,7α,12α)-3-amino-7,12-dihydroxycholan-24-oic acid-BBN(7-14) wherein the BBN(7-14) sequence is SEQ ID NO: 1.

42. A method of imaging comprising the steps of:
administering to a patient a diagnostic imaging agent comprising DO3A-monoamide-Gly-(3β,5β,7α,12α)-3-amino-7,12-dihydroxycholan-24-oic acid-BBN(7-14) complexed with a diagnostic radionuclide, wherein the BBN(7-14) sequence is SEQ ID NO: 1, and
imaging said patient.

43. A method for preparing a diagnostic imaging agent comprising the step of adding to an injectable medium a compound comprising DO3A-monoamide-Gly-4-aminobenzoic acid-BBN(7-14) wherein the BBN(7-14) sequence is SEQ ID NO: 1.

44. A method of treating a patient comprising the step of administering to a patient a radiotherapeutic agent comprising the compound of embodiment 41 complexed with a therapeutic radionuclide.

45. A method of preparing a radiotherapeutic agent comprising the step of adding to an injectable medium a substance comprising the compound of embodiment 41.

46. A compound DO3A-monoamide-Gly-4-aminobenzoic acid-BBN(7-14) wherein the BBN(7-14) sequence is SEQ ID NO: 1.

47. A method of imaging comprising the steps of:
administering to a patient a diagnostic imaging agent comprising DO3A-monoamide-Gly-4-aminobenzoic acid-BBN(7-14) complexed with a diagnostic radionuclide, wherein the BBN(7-14) sequence is SEQ ID NO: 1, and
imaging said patient.

48. A method for preparing a diagnostic imaging agent comprising the step of adding to an injectable medium a compound comprising DO3A-monoamide-Gly-4-aminobenzoic acid-BBN(7-14) wherein the BBN(7-14) sequence is SEQ ID NO: 1.

49. A method of treating a patient comprising the step of administering to a patient a radiotherapeutic agent comprising the compound of embodiment 46 complexed with a therapeutic radionuclide.

50. A method of preparing a radiotherapeutic agent comprising the step of adding to an injectable medium a substance comprising the compound of embodiment 46.

51. A compound of the general formula:

M-N—O—P-G
wherein
   M is an optical label or a metal chelator optionally complexed with a radionuclide;
   N is 0, an alpha amino acid, a non-alpha amino acid with a cyclic group or other linking group;
   O is an alpha amino acid or a non-alpha amino acid with a cyclic group;
   P is 0, an alpha amino acid, a non-alpha amino acid with a cyclic group, or other linking group; and
   G is a GRP receptor targeting peptide,
wherein at least one of N, O or P is a non-alpha amino acid with a cyclic group.

52. The compound of embodiment 51, wherein G is an agonist or a peptide which confers agonist activity.

53. The compound of embodiment 51, wherein the non-alpha amino acid with a cyclic group is selected from the group consisting of:
4-aminobenzoic acid;
4-aminomethyl benzoic acid;
trans-4-aminomethylcyclohexane carboxylic acid;
4-(2-aminoethoxy)benzoic acid;
isonipecotic acid;
2-aminomethylbenzoic acid;
4-amino-3-nitrobenzoic acid;
4-(3-carboxymethyl-2-keto-1-benzimidazolyl)-piperidine;
6-(piperazin-1-yl)-4-(3H)-quinazolinone-3-acetic acid;
)-5-amino-1,2,4,5,6,7-hexahydro-azepino[3,21-hi]indole-4-one-2-carboxylic acid;
)-4-amino-6-aza-5-oxo-9-thiabicyclo[4.3.0]nonane-7-carboxylic acid;
3-carboxymethyl-1-phenyl-1,3,8-triazaspiro[4.5]decan-4-one;
N1-piperazineacetic acid;
N-4-aminoethyl-N-1-acetic acid;
(3S)-3-amino-1-carboxymethylcaprolactam; and
,9)-6-amino-2-carboxymethyl-3,8-diazabicyclo-[4,3,0]-nonane-1,4-dione.

54. The compound of embodiment 51, wherein M is selected from the group consisting of: DTPA, DOTA, DO3A, HPDO3A, EDTA, and TETA.

55. The compound of embodiment 51, wherein M is selected from the group consisting of EHPG and derivatives thereof.

56. The compound of embodiment 51, wherein M is selected from the group consisting of 5-Cl-EHPG, 5-Br-EHPG, 5-Me-EHPG, 5-t-Bu-EHPG, and 5-sec-Bu-EHPG.

57. The compound of embodiment 51, wherein M is selected from the group consisting of benzodiethylenetriamine pentaacetic acid (benzo-DTPA) and derivatives thereof.

58. The compound of embodiment 51, wherein M is selected from the group consisting of dibenzo-DTPA, phenyl-DTPA, diphenyl-DTPA, benzyl-DTPA, and dibenzyl DTPA.

59. The compound of embodiment 51, wherein M is selected from the group consisting of HBED and derivatives thereof.

60. The compound of embodiment 51, wherein M is a macrocyclic compound which contains at least 3 carbon atoms and at least two heteroatoms (O and/or N), which macrocyclic compounds can consist of one ring, or two or three rings joined together at the hetero ring elements.

61. The compound of embodiment 51, wherein M is selected from the group consisting of benzo-DOTA, dibenzo-DOTA, and benzo-NOTA, benzo-TETA, benzo-DOTMA, and benzo-TETMA.

62. The compound of embodiment 51, wherein M is selected from the group consisting of derivatives of 1,3-propylenediaminetetraacetic acid (PDTA) and triethylenetetraaminehexaacetic acid (TTHA); derivatives of

1,5,10-N,N′,N″-tris(2,3-dihydroxybenzoyl)-tricatecholate (LICAM) and
1,3,5-N,N′,N″-tris(2,3-dihydroxybenzoyl)aminomethylbenzene (MECAM).

63. The compound of embodiment 51, selected from the group consisting of

-monoamide-Gly-4-aminobenzoic acid-BBN(7-14) wherein the BBN(7-14) sequence is SEQ ID NO: 1;
-monoamide-4-aminomethyl benzoic acid-BBN(7-14) wherein the BBN(7-14) sequence is SEQ ID NO: 1;
monoamide-trans-4-aminomethylcyclohexyl carboxylic acid-BBN(7-14) wherein the BBN(7-14) sequence is SEQ ID NO: 1;
-monoamide-4-(2-aminoethoxy)benzoic acid-BBN(7-14) wherein the BBN(7-14) sequence is SEQ ID NO: 1;
-monoamide-Gly-isonipecotic acid-BBN(7-14) wherein the BBN(7-14) sequence is SEQ ID NO: 1;
-monoamide-2-aminomethylbenzoic acid-BBN(7-14) wherein the BBN(7-14) sequence is SEQ ID NO: 1;
-monoamide-4-aminomethyl-3-nitrobenzoic acid-BBN(7-14) wherein the BBN(7-14) sequence is SEQ ID NO: 1;
-monoamide-8-amino-3,6-dioxaoctanoic acid-1-naphthylalanine-BBN(7-14) wherein the BBN(7-14) sequence is SEQ ID NO: 1;
-monoamide-4-(3-carboxymethyl-2-keto-1-benzimidazolyl-piperidine-BBN(7-14) wherein the BBN(7-14) sequence is SEQ ID NO: 1;
-monoamide-6-(piperazin-1-yl)-4-(3H)-quinazolinone-3-acetic acid-BBN(7-14) wherein the BBN(7-14) sequence is SEQ ID NO: 1;
-monoamide-(2S,5S)-5-amino-1,2,4,5,6,7-hexahydro-azepino[3,2,1-hi]indole-4-one-2
carboxylic acid-BBN(7-14) wherein the BBN(7-14) sequence is SEQ ID NO: 1;
   -monoamide-(4S,7R)-4-amino-6-aza-5-oxo-9-thiabicyclo[4.3.0]nonane-7-carboxylic acid-BBN(7-14) wherein the BBN(7-14) sequence is SEQ ID NO: 1;
   -monoamide-N,N-dimethylglycine-BBN(7-14) wherein the BBN(7-14) sequence is SEQ ID NO: 1;
   -monoamide-3-carboxymethyl-1-phenyl-1,3,8-triazaspiro[4.5]decan-4-one-BBN(7-14) wherein the BBN(7-14) sequence is SEQ ID NO: 1;
   -monoamide-N1-piperazineacetic acid-BBN(7-14) wherein the BBN(7-14) sequence is SEQ ID NO: 1;
   -monoamide-N-4-aminoethyl-N-1-piperazineacetic acid-BBN(7-14) wherein the BBN(7-14) sequence is SEQ ID NO: 1;
   -monoamide-(3S)-3-amino-1-carboxymethylcaprolactam-BBN(7-14) wherein the BBN(7-14) sequence is SEQ ID NO: 1;
   -monoamide-(2S,6S,9)-6-amino-2-carboxymethyl-3,8-diazabicyclo-[4,3,0]-nonane-1,4dione-BBN(7-14) wherein the BBN(7-14) sequence is SEQ ID NO: 1;
   -monoamide-5-aminopentanoic acid-trans-4-aminomethylcyclohexane-1-carboxylic acid-BBN(7-14) wherein the BBN(7-14) sequence is SEQ ID NO: 1;
   -monoamide-trans-4-aminomethylcyclohexane-1-carboxylic acid-D-phenylalanine-BBN(7-14) wherein the BBN(7-14) sequence is SEQ ID NO: 1;
   -monoamide-4-aminomethylbenzoic acid-8-amino-3,6-dioxaoctanoic acid-BBN(7-14) wherein the BBN(7-14) sequence is SEQ ID NO: 1;
   -monoamide-4-benzoyl-(L)-phenylalanine-trans-4-aminomethylcyclohexane-1-carboxylic acid-BBN(7-14) wherein the BBN(7-14) sequence is SEQ ID NO: 1;
   -monoamide-trans-4-aminomethylcyclohexane-1-carboxylic acid-Arg-BBN(7-14) wherein the BBN(7-14) sequence is SEQ ID NO: 1;
   -monoamide-trans-4-aminomethylcyclohexane-1-carboxylic acid-Lys-BBN(7-14) wherein the BBN(7-14) sequence is SEQ ID NO: 1;
   -monoamide-trans-4-aminomethylcyclohexane-1-carboxylic acid-diphenylalanine-BBN(7-14) wherein the BBN(7-14) sequence is SEQ ID NO: 1;
   -monoamide-trans-4-aminomethylcyclohexane-1-carboxylic acid-1-naphthylalanine-BBN(7-14) wherein the BBN(7-14) sequence is SEQ ID NO: 1;
   -monoamide-trans-4-aminomethylcyclohexane-1-carboxylic acid-8-amino-3,6-dioxaoctanoic acid-BBN(7-14) wherein the BBN(7-14) sequence is SEQ ID NO: 1;
   -monoamide-trans-4-aminomethylcyclohexane-1-carboxylic acid-Ser-BBN(7-14) wherein the BBN(7-14) sequence is SEQ ID NO: 1;
   -monoamide-trans-4-aminomethylcyclohexane-1-carboxylic acid-2,3-diaminopropionic acid-BBN(7-14) wherein the BBN(7-14) sequence is SEQ ID NO: 1;
   -monoamide-trans-4-aminomethylcyclohexane-1-carboxylic acid-biphenylalanine-BBN(7-14) wherein the BBN(7-14) sequence is SEQ ID NO: 1;
   -monoamide-trans-4-aminomethylcyclohexane-1-carboxylic acid-(2S,5S)-5-amino-1,2,4,5,6,7-hexahydro-azepino[3,21-hi]indole-4-one-2-carboxylic acid-BBN(7-14) wherein the BBN(7-14) sequence is SEQ ID NO: 1;
   -monoamide-trans-4-aminomethylcyclohexane-1-carboxylic acid-trans-4-aminomethylcyclohexane-1-carboxylic acid-BBN(7-14) wherein the BBN(7-14) sequence is SEQ ID NO: 1;
   -monoamide-8-amino-3,6-dioxaoctanoic acid-phenylalanine-BBN(7-14) wherein the BBN(7-14) sequence is SEQ ID NO: 1;
   -monoamide-trans-4-aminomethylcyclohexane-1-carboxylic acid-phenylalanine-BBN(7-14) wherein the BBN(7-14) sequence is SEQ ID NO: 1;
   -monoamide-8-aminooctanoic acid-trans-4-aminomethylcyclohexane-1-carboxylic acid-BBN(7-14) wherein the BBN(7-14) sequence is SEQ ID NO: 1;
   -monoamide-4′-aminomethyl-biphenyl-1-carboxylic acid-BBN(7-14) wherein the BBN(7-14) sequence is SEQ ID NO: 1;
   -monoamide-3′-aminomethyl-biphenyl-3-carboxylic acid-BBN(7-14) wherein the BBN(7-14) sequence is SEQ ID NO: 1;
   TA-Gly-4-aminobenzoic acid-BBN(7-14) wherein the BBN(7-14) sequence is SEQ ID NO: 1;
   -monoamide-4-aminomethylphenoxyacetic acid-BBN(7-14) wherein the BBN(7-14) sequence is SEQ ID NO: 1;
   -monoamide-Gly-4-aminophenylacetic acid-BBN(7-14) wherein the BBN(7-14) sequence is SEQ ID NO: 1;
   3A-4-phenoxy-BBN(7-14) wherein the BBN(7-14) sequence is SEQ ID NO: 1;
   -monoamide-3-aminomethylbenzoic acid-BBN(7-14) wherein the BBN(7-14) sequence is SEQ ID NO: 1;
   -monoamide-4-aminomethylphenylacetic acid-BBN(7-14) wherein the BBN(7-14) sequence is SEQ ID NO: 1;
   -monoamide-4-aminomethyl-3-methoxybenzoic acid-BBN(7-14) wherein the BBN(7-14) sequence is SEQ ID NO: 1;
   Boa-Gly-4-aminobenzoic acid-BBN(7-14) wherein the BBN(7-14) sequence is SEQ ID NO: 1;
   DO3A-monoamide-Gly-4-hydrazinobenzoyl-BBN(7-14) wherein the BBN(7-14) sequence is SEQ ID NO: 1;
   DO3A-monoamide-4-aminobenzoic acid-BBN(7-14) wherein the BBN(7-14) sequence is SEQ ID NO: 1;
   DO3A-monoamide-4-aminobenzoic acid-Gly-BBN(7-14) wherein the BBN(7-14) sequence is SEQ ID NO: 1;
   DO3A-monoamide-Gly-6-Aminonicotinic acid-BBN(7-14) wherein the BBN(7-14) sequence is SEQ ID NO: 1;
   DO3A-monoamide-Gly-4′-Amino-2′-methyl biphenyl-4-carboxylic acid-BBN(7-14) wherein the BBN(7-14) sequence is SEQ ID NO: 1;
   DO3A-monoamide-Gly-3′-Aminobiphenyl-3-carboxylic acid-BBN(7-14) wherein the BBN(7-14) sequence is SEQ ID NO: 1;
   DO3A-monoamide-Gly-1,2-diaminoethyl-Terephthalic acid-BBN(7-14) wherein the BBN(7-14) sequence is SEQ ID NO: 1;
   DO3A-monoamide-Gly-Gly-4-aminobenzoic acid-BBN(7-14) wherein the BBN(7-14) sequence is SEQ ID NO: 1;
   DO3A-monoamide-G-4-aminobenzoic acid-EWAVGHLM-NH₂ wherein EWAVGHLM-NH₂ is SEQ ID NO: 2;
   DO3A-monoamide-G-4-aminobenzoic acid-QWAVGHLM-OH wherein QWAVGHLM-OH is SEQ ID NO: 1;
   DO3A-monoamide-G-4-aminobenzoic acid-(D)-Phe-BBN(7-14) wherein the BBN(7-14) sequence is SEQ ID NO: 1;
   DO3A-monoamide-G-4-aminobenzoic acid-QRLGNQWAVGHLM-NH₂ wherein QRLGNQWAVGHLM-NH₂ is SEQ ID NO: 3;
   DO3A-monoamide-G-4-aminobenzoic acid-QRYGNQWAVGHLM-NH₂ wherein QRYGNQWAVGHLM-NH₂ is SEQ ID NO: 4;
   DO3A-monoamide-G-4-aminobenzoic acid-QKYGNQWAVGHLM-NH₂ wherein QKYGNQWAVGHLM-NH₂ is SEQ ID NO: 5;
   DO3A-monoamide-G-4-aminobenzoic acid-(D)-Phe-QWAVGHL-NH-Pentyl wherein QWAVGHL-NH-Pentyl is SEQ ID NO: 6;
   DO3A-monoamide-G-4-aminobenzoic acid-QWSVaHLM-NH₂ wherein QWSVaHLM-NH₂ is SEQ ID NO: 7;
   DO3A-monoamide-G-4-aminobenzoic acid-(D)-Phe-QWAVGHLL-NH₂ wherein QWAVGHLL-NH₂ is SEQ ID NO: 8;
   DO3A-monoamide-G-4-aminobenzoic acid-(D)-Tyr-QWAV-Bala-HF-Nle-NH₂ wherein QWAV-Bala-HF-Nle-NH₂ is SEQ ID NO: 9;
   DO3A-monoamide-G-4-aminobenzoic acid-Phe-QWAV-Bala-HF-Nle-NH₂ wherein QWAV-Bala-HF-Nle-NH₂ is SEQ ID NO: 9;
   DO3A-monoamide-G-4-aminobenzoic acid-QWAGHFL-NH₂ wherein QWAGHFL-NH₂ is SEQ ID NO: 10;
   DO3A-monoamide-G-4-aminobenzoic acid-LWAVGSFM-NH₂ wherein LWAVGSFM-NH₂ is SEQ ID NO: 11;
   DO3A-monoamide-G-4-aminobenzoic acid-HWAVGHLM-NH₂ wherein HWAVGHLM-NH₂ is SEQ ID NO: 12;
   DO3A-monoamide-G-4-aminobenzoic acid-LWAVGSFM-NH₂ wherein LWAVGSFM-NH₂ is SEQ ID NO: 11;
   DO3A-monoamide-G-4-aminobenzoic acid-QWAVGHFM-NH₂ wherein QWAVGHFM-NH₂ is SEQ ID NO: 13;
   DO3A-monoamide-Gly-3-aminobenzoic acid-BBN(7-14) wherein the BBN(7-14) sequence is SEQ ID NO: 1;
   DO3A-monoamide-Gly-6-aminonaphthoic acid-BBN(7-14) wherein the BBN(7-14) sequence is SEQ ID NO: 1;
   DO3A-monoamide-Gly-4-methylaminobenzoic acid-BBN(7-14) wherein the BBN(7-14) sequence is SEQ ID NO: 1;
   Cm4 pm10d2a-Gly-4-aminobenzoic acid-BBN(7-14) wherein the BBN(7-14) sequence is SEQ ID NO: 1;
   N,N-dimethylglycine-Ser-Cys(Acm)-Gly-Gly-4-aminobenzoic acid-BBN(7-14) wherein the BBN(7-14) sequence is SEQ ID NO: 1;
   N,N-dimethylglycine-Ser-Cys(Acm)-Gly-Gly-3-amino-3-deoxycholic acid-BBN(7-14) wherein the BBN(7-14) sequence is SEQ ID NO: 1;
   DO3A-monoamide-Gly-3-methoxy-4-aminobenzoic acid-BBN(7-14) wherein the BBN(7-14) sequence is SEQ ID NO: 1;
   DO3A-monoamide-Gly-3-chloro-4-aminobenzoic acid-BBN(7-14) wherein the BBN(7-14) sequence is SEQ ID NO: 1;
   DO3A-monoamide-Gly-3-methyl-4-aminobenzoic acid-BBN(7-14) wherein the BBN(7-14) sequence is SEQ ID NO: 1
   DO3A-monoamide-Gly-3-hydroxy-4-aminobenzoic acid-BBN(7-14) wherein the BBN(7-14) sequence is SEQ ID NO: 1;
   (DO3A-monoamide)₂-N,N′-Bis(2-aminoethyl)-succinamic acid-BBN(7-14) wherein the BBN(7-14) sequence is SEQ ID NO: 1;
   DO3A-monoamide-G-4-aminobenzoic acid-QWAVGHFL-NH₂ wherein QWAVGHFL-NH₂ is SEQ ID NO: 22
   DO3A-monoamide-4-aminomethylbenzoic acid-L-1-Naphthylalanine-QWAVGHLM-NH₂ wherein QWAVGHLM-NH₂ is SEQ ID NO: 1; and
   DO3A-monoamide-G-4-aminobenzoic acid-QWAVGNMeHLM-NH₂ wherein QWAVGNMeHLM-NH₂ is SEQ ID NO: 15.

64. The compound of any one of embodiments 51, 52 or 53, wherein the optical label is selected from the group consisting of organic chromophores, organic fluorophores, light-absorbing compounds, light-reflecting compounds, light-scattering compounds, and bioluminescent molecules.

65. A method of imaging comprising the steps of:
administering to a patient a diagnostic imaging agent comprising the compound of embodiment 51 wherein M is a metal chelator complexed with a diagnostic radionuclide, and
imaging said patient.

66. A method of imaging comprising the steps of: administering to a patient a diagnostic imaging agent comprising the compound of embodiment 63, and imaging said patient.

67. A method of imaging comprising the steps of: administering to a patient a diagnostic imaging agent comprising the compound of embodiment 51, wherein M is an optical label, and imaging said patient.

68. A method for preparing a diagnostic imaging agent comprising the step of adding to an injectable medium a substance comprising the compound of embodiment 51.

69. A method of treating a patient comprising the step of administering to a patient a radiotherapeutic agent comprising the compound of embodiment 51 complexed with a therapeutic radionuclide.

70. A method of preparing a radiotherapeutic agent comprising the step of adding to an injectable medium a substance comprising the compound of embodiment 51.

71. A method of synthesizing DO3A-monoamide-Gly-(3β,5β,7α,12α)-3-amino-7,12-dihydroxycholan-24-oic acid-BBN(7-14) wherein the BBN(7-14) sequence is SEQ ID NO: 1 comprising the steps of:
(a) shaking a solution in a solid phase peptide synthesis vessel, said solution comprising a resin and at least one peptide building ingredient,
(b) flushing said solution, and
(c) washing said resin with DMA,
wherein said at least one peptide building ingredient includes DMA morpholine, (3β,5β,7α,12α)-3-[[(9H-fluoren-9-ylmethoxy)amino]acetyl]amino-7,12-dihydroxycholan-24-oic acid, HOBt, DIC, HATU or mixtures thereof, and
wherein each of steps (a), (b) and (c) are repeated until the compound DO3A-monoamide-Gly-(3β,5β,7α,12α)-3-amino-7,12-dihydroxycholan-24-oic acid-BBN(7-14) wherein the BBN(7-14) sequence is SEQ ID NO: 1 is obtained.

72. A method of synthesizing DO3A-monoamide-Gly-4-aminobenzoic acid-BBN(7-14) wherein the BBN(7-14) sequence is SEQ ID NO: 1 comprising the steps of:
(a) shaking a solution in a solid phase peptide synthesis vessel or reaction block, said solution comprising a resin and at least one peptide building ingredient,
(b) flushing said solution, and
(c) washing said resin with DMA,
wherein said at least one peptide building ingredient includes DMA, morpholine, Fmoc-4-aminobenzoic acid, HOBt, DIC, HBTU, HATU or mixtures thereof, and
wherein each of steps (a), (b) and (c) are repeated until the compound DO3A-monoamide-Gly-4-aminobenzoic acid-BBN(7-14) wherein the BBN(7-14) sequence is SEQ ID NO: 1 is obtained.

73. A method for labeling DO3A-monoamide-Gly-(3β,5β,7α,12α)-3-amino-7,12-dihydroxycholan-24-oic acid-BBN(7-14) wherein the BBN(7-14) sequence is SEQ ID NO: 1 comprising the steps of
incubating a first solution comprising
   DO3A-monoamide-Gly-(3β,5β,7α,12α)-3-amino-7,12-dihydroxycholan-24-oic acid-BBN(7-14) wherein the BBN(7-14) sequence is SEQ ID NO: 1,
   ammonium acetate,
   a radioactive metal precursor selected from the group consisting of ¹⁷⁷LuCl₃ or ¹¹¹InCl₃,
   HCl, and
adding to said first solution a second solution comprising Na₂EDTA.2H₂O and water to obtain a radiochemical purity greater than 95%.

74. A method for labeling DO3A-monoamide-Gly-4-aminobenzoic acid-BBN(7-14) wherein the BBN(7-14) sequence is SEQ ID NO: 1 comprising the steps of
incubating a first solution comprising
   DO3A-monoamide-Gly-4-aminobenzoic acid-BBN(7-14) wherein the BBN(7-14) sequence is SEQ ID NO: 1,
   ammonium acetate,
   a radioactive metal precursor selected from the group consisting of ¹⁷⁷LuCl₃ or ¹¹InCl₃,
   HCl, and
adding to said first solution a second solution comprising Na₂EDTA.2H₂O and water to obtain a radiochemical purity greater than 95%.

75. A method of synthesizing DO3A-monoamide-Gly-(3β,5β,7α,12α)-3-amino-7,12-dihydroxycholan-24-oic acid-BBN(7-14) wherein the BBN(7-14) sequence is SEQ ID NO: 1 by coupling of individual amino acids, protected amino acids or modified amino acids, with any required additional treatments with reagents or processing steps before or after the coupling steps in solution.

76. A method of synthesizing DO3A-monoamide-Gly-4-aminobenzoic acid-BBN(7-14) wherein the BBN(7-14) sequence is SEQ ID NO: 1 by segment coupling of modified, protected, unprotected or otherwise variable peptide fragments combined with any required additional treatments with reagents or processing steps before or after the coupling steps in solution or on solid phase or via a combined solution and solid phase synthesis steps and methods.

77. A method of synthesizing DO3A-monoamide-Gly-(3β,5β,7α,12α)-3-amino-7,12-dihydroxycholan-24-oic acid-BBN(7-14) wherein the BBN(7-14) sequence is SEQ ID NO: 1 by coupling of individual amino acids protected amino acids or modified amino acids, with any required additional treatments with reagents or processing steps before or after the coupling steps in solution.

78. A compound of the general formula:

M-N—O—P-G
wherein
   M is DO3A, optionally complexed with a radionuclide;
   N is 0, an alpha or non-alpha amino acid or other linking group;
   O is an alpha or non-alpha amino acid; and
   P is 0, an alpha or non-alpha amino acid or other linking group,
   and G is a GRP receptor targeting peptide,
      wherein at least one of N, O or P is 8-amino-3,6-dioxaoctanoic acid.

79. The compound of embodiment 78, wherein the GRP receptor targeting peptide is selected from the group consisting of QWAVGHLM-OH (SEQ ID NO: 1), QWAVGHLM-NH₂ (SEQ ID NO: 1), QWAVGNMeHLM-NH₂ (SEQ ID NO: 15), QWAVGHFL-NH₂ (SEQ ID NO: 22), QRLGNQWAVGHLM-NH₂ (SEQ ID NO: 3), QRYGNQWAVGHLM-NH₂ (SEQ ID NO: 4), QKYGNQWAVGHLM-NH₂ (SEQ ID NO: 5), QWAVGHL-NH-Pentyl (SEQ ID NO: 6), QWSVaHLM-NH₂ (SEQ ID NO: 7), QWAVGHLL-NH₂ (SEQ ID NO: 8), QWAV-Bala-HF-Nle-NH₂ (SEQ ID NO: 9), QWAGHFL-NH₂ (SEQ ID NO: 10), LWAVGSFM-NH₂ (SEQ ID NO: 11), HWAVGHLM-NH₂ (SEQ ID NO: 12), LWATGSFM-NH₂ (SEQ ID NO: 17), LWAVGSFM-NH₂ (SEQ ID NO: 11), QWAVaHLM-NH₂ (SEQ ID NO: 14), and QWAVGHFM-NH₂ (SEQ ID NO: 13).

80. A compound of the general formula:

M-N—O—P-G
wherein
   M is DO3A, optionally complexed with a radionuclide;
   N is 0, an alpha or non-alpha amino acid or other linking group;
   O is an alpha or non-alpha amino acid; and
   P is 0, an alpha or non-alpha amino acid or other linking group,
   and G is a GRP receptor targeting peptide,
wherein at least one of N, O or P is (3β,5β,12α)-3-amino-12-hydroxycholan-24-oic acid.

81. The compound of embodiment 80 wherein the GRP receptor targeting peptide is selected from the group consisting of QWAVGHLM-OH (SEQ ID NO: 1), QWAVGHLM-NH₂ (SEQ ID NO: 1), QWAVGNMeHLM-NH₂ (SEQ ID NO: 15), QWAVGHFL-NH₂ (SEQ ID NO: 22), QRLGNQWAVGHLM-NH₂ (SEQ ID NO: 3), QRYGNQWAVGHLM-NH₂ (SEQ ID NO: 4), QKYGNQWAVGHLM-NH₂ (SEQ ID NO: 5), QWAVGHL-NH-Pentyl (SEQ ID NO: 6), QWSVaHLM-NH₂ (SEQ ID NO: 7), QWAVGHLL-NH₂ (SEQ ID NO: 8), QWAV-Bala-HF-Nle-NH₂ (SEQ ID NO: 9) QWAGHFL-NH₂ (SEQ ID NO: 10), LWAVGSFM-NH₂ (SEQ ID NO: 11), HWAVGHLM-NH₂ (SEQ ID NO: 12), LWATGSFM-NH₂ (SEQ ID NO: 17), LWAVGSFM-NH₂ (SEQ ID NO: 11), QWAVaHLM-NH₂ (SEQ ID NO: 14), and QWAVGHFM-NH₂ (SEQ ID NO: 13).

82. A compound of the general formula:

M-N—O—P-G
wherein
   M is DO3A, optionally complexed with a radionuclide;
   N is 0, an alpha or non-alpha amino acid or other linking group;
   O is an alpha or non-alpha amino acid; and
   P is 0, an alpha or non-alpha amino acid or other linking group,
   and G is a GRP receptor targeting peptide,
wherein at least one of N, O or P is 4-aminobenzoic acid.

83. The compound of embodiment 82, wherein the GRP receptor targeting peptide is selected from the group consisting of QWAVGHLM-OH (SEQ ID NO: 1), QWAVGHLM-NH₂ (SEQ ID NO: 1), QWAVGNMeHLM-NH₂ (SEQ ID NO: 15), QWAVGHFL-NH₂ (SEQ ID NO: 22), QRLGNQWAVGHLM-NH₂ (SEQ ID NO: 3), QRYGNQWAVGHLM-NH₂ (SEQ ID NO: 4), QKYGNQWAVGHLM-NH₂ (SEQ ID NO: 5), QWAVGHL-NH-Pentyl (SEQ ID NO: 6), QWSVaHLM-NH₂ (SEQ ID NO: 7), QWAVGHLL-NH₂ (SEQ ID NO: 8), QWAV-Bala-HF-Nle-NH₂ (SEQ ID NO: 9) QWAGHFL-NH₂ (SEQ ID NO: 10), LWAVGSFM-NH₂ (SEQ ID NO: 11), HWAVGHLM-NH₂ (SEQ ID NO: 12), LWATGSFM-NH₂ (SEQ ID NO: 17), LWAVGSFM-NH₂ (SEQ ID NO: 11), QWAVaHLM-NH₂ (SEQ ID NO: 14), QWAVGHFM-NH₂ (SEQ ID NO: 13), Nme-QWAVGHLM-NH₂ wherein QWAVGHLM-NH₂ is (SEQ ID NO: 1), Q-[Ψ[CSNH]WAVGHLM-NH₂, Q-Ψ[CH₂NH]-WAVGHLM-NH₂, Q-Ψ[CH═CH]WAVGHLM-NH₂, α-MeQWAVGHLM-NH₂, QNme-WAVGHLM-NH₂, QW-Ψ[CSNH]-AVGHLM-NH₂, QW-Ψ[CH₂NH]-AVGHLM-NH₂, QW-Ψ[CH═CH]-AVGHLM-NH₂, Q-α-Me-WAVGHLM-NH₂, QW-Nme-AVGHLM-NH₂, QWA=Ψ[CSNH]-VGHLM-NH₂, QWA-Ψ[CH₂NH]-VGHLM-NH₂, QW-Aib-VGHLM-NH₂, QWAV-Sar-HLM-NH₂, QWAVG-Ψ[CSNH]-HLM-NH₂, QWAVG-Ψ[CH═CH]-HLM-NH₂, QWAV-Dala-HLM-NH₂, QWAVG-Nme-His-LM-NH₂, QWAVG-H-Ψ[CSNH]-L-M-NH₂, QWAVG-H-Ψ[CH₂NH]-LM-NH₂, QWAVGH-ψ[CH═CH]-LM-NH₂, QWAVG-α-Me-HLM-NH₂, QWAVGH-Nme-LM-NH₂, QWAVGH-α-MeLM-NH₂, QWAVGHF-L-NH₂ and QWAVGHLM-NH₂, wherein QWAVGHLM-NH₂ is SEQ ID NO: 1 and QWAVGHFL-NH₂ is SEQ ID NO: 22.

84. A method of phototherapy comprising administering to a patient a compound of any one of embodiments 1, 20 or 51 wherein M is an optical label useful in phototherapy.

85. A compound selected from the group consisting of:

DO3A-monoamide-G-4-aminobenzoic acid-QWAVaHLM-NH₂ wherein QWAVaHLM-NH₂ is SEQ ID NO: 14;
DO3A-monoamide-G-4-aminobenzoic acid-fQWAVGHLM-NH₂ wherein QWAVGHLM-NH₂ is SEQ ID NO: 1;
DO3A-monoamide-G-4-aminobenzoic acid-fQWAVGHLL-NH₂ wherein QWAVGHLL-NH₂ is SEQ ID NO: 8;
DO3A-monoamide-G-4-aminobenzoic acid-fQWAVGHL-NH-pentyl wherein QWAVGHL-NH-pentyl is SEQ ID NO: 6;
DO3A-monoamide-G-4-aminobenzoic acid-yQWAV-Bala-HFNle-NH₂ wherein QWAV-Bala-HFNle-NH₂ is SEQ ID NO: 9;
DO3A-monoamide-G-4-aminobenzoic acid-fQWAV-Bala-HFNle-NH₂ wherein QWAV-Bala-HFNle-NH₂ is SEQ ID NO: 9;
DO3A-monoamide-G-4-aminobenzoic acid-QWAVGHFL-NH₂ wherein QWAVGHFL-NH₂ is SEQ ID NO: 22;
DO3A-monoamide-G-4-aminobenzoic acid-QWAVGNMeHisLM-NH₂ wherein QWAVGNMeHisLM-NH₂ is SEQ ID NO: 15;
DO3A-monoamide-G-4-aminobenzoic acid-LWAVGSFM-NH₂ wherein LWAVGSFM-NH₂ is SEQ ID NO: 11;
DO3A-monoamide-G-4-aminobenzoic acid-HWAVGHLM-NH₂ wherein HWAVGHLM-NH₂ is SEQ ID NO: 12;
DO3A-monoamide-G-4-aminobenzoic acid-LWATGHFM-NH₂ wherein LWATGHFM-NH₂ is SEQ ID NO: 16;
DO3A-monoamide-G-4-aminobenzoic acid-QWAVGHFM-NH₂ wherein QWAVGHFM-NH₂ is SEQ ID NO: 13;
DO3A-monoamide-G-4-aminobenzoic acid-QRLGNQWAVGHLM-NH₂ wherein QRLGNQWAVGHLM-NH₂ is SEQ ID NO: 3;
DO3A-monoamide-G-4-aminobenzoic acid-QRYGNQWAVGHLM-NH₂ wherein QRYGNQWAVGHLM-NH₂ is SEQ ID NO: 4;
DO3A-monoamide-G-4-aminobenzoic acid-QKYGNQWAVGHLM-NH₂ wherein QKYGNQWAVGHLM-NH₂ is SEQ ID NO: 5;
Pglu-Q-Lys(DO3A-monoamide-G-4-aminobenzoic acid)-LGNQWAVGHLM-NH₂ wherein LGNQWAVGHLM-NH₂ is SEQ ID NO: 18;
DO3A-monoamide-G-3-amino-3-deoxycholic acid-QWAVaHLM-NH₂ wherein QWAVaHLM-NH₂ is SEQ ID NO: 14;
DO3A-monoamide-G-3-amino-3-deoxycholic acid-fQWAVGHLM-NH₂ wherein QWAVGHLM-NH₂ is SEQ ID NO: 1;
DO3A-monoamide-G-3-amino-3-deoxycholic acid-fQWAVGHLL-NH₂ wherein QWAVGHLL-NH₂ is SEQ ID NO: 8;
DO3A-monoamide-G-3-amino-3-deoxycholic acid-fQWAVGHL-NH-pentyl wherein QWAVGHL-NH-pentyl is SEQ ID NO: 6;
DO3A-monoamide-G-3-amino-3-deoxycholic acid-yQWAV-Bala-HFNle-NH₂ wherein QWAV-Bala-HFNle-NH₂ is SEQ ID NO: 9;
DO3A-monoamide-G-3-amino-3-deoxycholic acid-fQWAV-Bala-HFN1e-NH₂ wherein QWAV-Bala-HFNle-NH₂ is SEQ ID NO: 9;
DO3A-monoamide-G-3-amino-3-deoxycholic acid-QWAVGHFL-NH₂ wherein QWAVGHFL-NH₂ is SEQ ID NO: 22
DO3A-monoamide-G-3-amino-3-deoxycholic acid-QWAVGNMeHLMNH₂ wherein QWAVGNMeHLMNH₂ is SEQ ID NO: 15;
DO3A-monoamide-G-3-amino-3-deoxycholic acid-LWAVGSFM-NH₂ wherein LWAVGSFM-NH₂ is SEQ ID NO: 11;
DO3A-monoamide-G-3-amino-3-deoxycholic acid-HWAVGHLM-NH₂ wherein HWAVGHLM-NH₂ is SEQ ID NO: 12;
DO3A-monoamide-G-3-amino-3-deoxycholic acid-LWATGHFM-NH₂ wherein LWATGHFM-NH₂ is SEQ ID NO: 16;
DO3A-monoamide-G-3-amino-3-deoxycholic acid-QWAVGHFM-NH₂ wherein QWAVGHFM-NH₂ is SEQ ID NO: 13
DO3A-monoamide-G-3-amino-3-deoxycholic acid-QRLGNQWAVGlyHLM-NH₂ wherein QRLGNQWAVGHLM-NH₂ is SEQ ID NO: 3;
DO3A-monoamide-G-3-amino-3-deoxycholic acid-QRYGNQWAVGHLM-NH₂ wherein QRYGNQWAVGHLM-NH₂ is SEQ ID NO: 4;
DO3A-monoamide-G-3-amino-3-deoxycholic acid-QKYGNQWAVGHLM-NH₂ wherein QKYGNQWAVGHLM-NH₂ is SEQ ID NO: 5; and
Pglu-Q-Lys(DO3A-monoamide-G-3-amino-3-deoxycholic acid)-LGNQWAVGHLM-NH₂ wherein LGNQWAVGHLM-NH₂ is SEQ ID NO: 18.

86. The method of any one of embodiments 16, 17, 39, 44, 49 or 69 further comprising administering a chemotherapeutic or other therapeutic agent.

87. A compound of any one of embodiments 78 or 80, wherein the GRP receptor targeting peptide is selected from the group consisting of Nme-QWAVGHLM-NH₂, Q-Ψ[CSNH]WAVGHLM-NH₂, Q-Ψ[CH₂NH]-WAVGHLM-NH₂, Q-Ψ[CH═CH]WAVGHLM-NH₂, α-MeQWAVGHLM-NH₂, QNme-WAVGHLM-NH₂, QW-Ψ[CSNH]-AVGHLM-NH₂, QW-Ψ[CH₂NH]-AVGHLM-NH₂, QW-Ψ[CH═CH]-AVGHLM-NH₂, Q-α-Me-WAVGHLM-NH₂, QW-Nme-AVGHLM-NH₂, QWA=Ψ[CSNH]-VGHLM-NH₂, QWA-Ψ[CH₂NH]-VGHLM-NH₂, QW-Aib-VGHLM-NH₂, QWAV-Sar-HLM-NH₂, QWAVG-Ψ[CSNH]-HLM-NH₂, QWAVG-Ψ[CH═CH]-HLM-NH₂, QWAV-Dala-HLM-NH₂, QWAVG-Nme-His-LM-NH₂, QWAVG-H-Ψ[CSNH]-L-M-NH₂, QWAVG-H-Ψ[CH₂NH]-LM-NH₂, QWAVGH-Ψ[CH═CH]-LM-NH₂, QWAVG-α-Me-HLM-NH₂, QWAVGH-Nme-LM-NH₂, QWAVGH-α-MeLM-NH₂, QWAVGHF-L-NH₂ and QWAVGHLM-NH₂, wherein QWAVGHLM-NH₂ is SEQ ID NO: 1 and QWAVGHFL-NH₂ is SEQ ID NO: 22.

88. A method for targeting the gastrin releasing peptide receptor (GRP-R) and neuromedin-B receptor (NMB-R), said method comprising administering a compound of the general formula:

M-N—O—P-G
wherein
   M is an optical label or a metal chelator, optionally complexed with a radionuclide;
   N is 0, an alpha or non-alpha amino acid or other linking group;
   O is an alpha or non-alpha amino acid; and
   P is 0, an alpha or non-alpha amino acid or other linking group,
   and G is a GRP receptor targeting peptide,
wherein at least one of N, O or P is a non-alpha amino acid.

89. The method of embodiment 88, wherein at least one of N, O or P is a non-alpha amino acid with a cyclic group.

90. The method of embodiment 89, wherein N is Gly, O is 4-aminobenzoic acid and P is none.

91. A method of targeting the GRP-R and the NMB-R, said method comprising administering a compound of the general formula:

M-N—O—P-G
wherein
   M is an optical label or a metal chelator, optionally complexed with a radionuclide;
   N is 0, an alpha amino acid, a substituted bile acid or other linking group;
   O is an alpha amino acid or a substituted bile acid; and
   P is 0, an alpha amino acid, a substituted bile acid or other linking group; and
   G is a GRP receptor targeting peptide, and
      wherein at least one of N, O or P is a substituted bile acid.

92. The method of embodiment 91, wherein N is Gly, O is (3β,5β,7α,12α)-3-amino-7,12-dihydroxycholan-24-oic acid, and P is none.

93. The method of any one of embodiments 88, 89 or 91, wherein the GRP receptor targeting peptide is selected from the group consisting of:

Nme-QWAVGHLM-NH₂,
Q-Ψ[CSNH]WAVGHLM-NH₂,
Q-Ψ[CH₂NH]-WAVGHLM-NH₂,
Q-Ψ[CH═CH]WAVGHLM-NH₂,
α-MeQWAVGHLM-NH₂,
QNme-WAVGHLM-NH₂,
QW-Ψ[CSNH]-AVGHLM-NH₂,
QW-Ψ[CH₂NH]-AVGHLM-NH₂,
QW-Ψ[CH═CH]-AVGHLM-NH₂,
Q-α-Me-WAVGHLM-NH₂,
QW-Nme-AVGHLM-NH₂,
QWA=Ψ[CSNH]-VGHLM-NH₂,
QWA-Ψ[CH₂NH]-VGHLM-NH₂,
QW-Aib-VGHLM-NH₂,
QWAV-Sar-HLM-NH₂,
QWAVG-Ψ[CSNH]-HLM-NH₂,
QWAVG-Ψ[CH═CM-HLM-NH₂,
QWAV-Dala-HLM-NH₂,
QWAVG-Nme-His-LM-NH₂,
QWAVG-H-Ψ[CSNH]-L-M-NH₂,
QWAVG-H-Ψ[CH₂NH]-LM-NH₂,
QWAVGH-Ψ[CH═CH]-LM-NH₂,
QWAVG-α-Me-HLM-NH₂,
QWAVGH-Nme-LM-NH₂,
and
QWAVGH-α-MeLM-NH₂

wherein QWAVGHLM-NH₂ is SEQ ID NO: 1 and QWAVGHFL-NH₂ is SEQ ID NO: 22.

94. A method of improving the in vivo activity of a compound of any one of embodiments 1, 20, 51, 78, 80, or 82, comprising the step of modifying the GRP receptor targeting peptide so as to reduce proteolytic cleavage of said peptide.

95. The method of embodiment 94, wherein the modified GRP-R targeting peptide is an agonist.

96. A method of reducing proteolytic cleavage of a gastrin releasing peptide (GRP) analogue of any one of embodiments 1, 20, 51, 78, 80, or 82, said method comprising the step of modifying the peptide bond in the GRP-R targeting moiety.

97. The method of embodiment 96, wherein the modified GRP-R targeting peptide is an agonist.

98. A method of reducing proteolytic cleavage of a gastrin releasing peptide (GRP) analogue having a gastrin releasing peptide receptor (GRP-R) targeting moiety that is an agonist, said method comprising the step of modifying the peptide bond in the GRP-R targeting moiety.

99. The method of any one of embodiments 94, 96 or 98, wherein the GRP-R targeting moiety is selected from the group consisting of:

Nme-QWAVGHLM-NH₂,
Q-Ψ[CSNH]WAVGHLM-NH_{2,}
Q-Ψ[CH₂NH]-WAVGHLM-NH₂,
Q-Ψ[CH═CH]WAVGHLM-NH₂,
α-MeQWAVGHLM-NH₂,
QNme-WAVGHLM-NH₂,
QW-Ψ[CSNH]-AVGHLM-NH₂,
QW-Ψ[CH₂NH]-AVGHLM-NH₂,
QW-Ψ[CH═CH]-AVGHLM-NH₂,
Q-α-Me-WAVGHLM-NH₂,
QW-Nme-AVGHLM-NH₂,
QWA=Ψ[CSNH]-VGHLM-NH₂,
QWA-Ψ[CH₂NH]-VGHLM-NH₂,
QW-Aib-VGHLM-NH₂,
QWAV-Sar-HLM-NH₂,
QWAVG-Ψ[CSNH]-HLM-NH₂,
QWAVG-Ψ[CH═CM-HLM-NH₂,
QWAV-Dala-HLM-NH₂,
QWAVG-Nme-His-LM-NH₂,
QWAVG-H-Ψ[CSNH]-L-M-NH₂,
QWAVG-H-Ψ[CH₂NH]-LM-NH₂,
QWAVGH-Ψ[CH═CH]-LM-NH₂,
QWAVG-α-Me-HLM-NH₂,
QWAVGH-Nme-LM-NH₂,
and
QWAVGH-α-MeLM-NH₂

wherein QWAVGHLM-NH₂ is SEQ ID NO: 1 and QWAVGHFL-NH₂ is SEQ ID NO: 22.

100. A compound according to any one of embodiments 1, 20, 51, 78, 80, or 82, wherein G is a GRP receptor targeting peptide that has been modified so as to reduce proteolytic cleavage.

101. A method of conferring specificity for the GRP-R and/or the NMB-R on a compound comprising an optical label or metal chelator optionally complexed with a radionuclide and a GRP-R targeting peptide, comprising including in such compound a linker of the general formula:

N—O—P
wherein
   N is 0, an alpha or non-alpha amino acid or other linking group;
   O is an alpha or non-alpha amino acid; and
   P is 0, an alpha or non-alpha amino acid or other linking group,
      wherein at least one of N, O or P is a non-alpha amino acid.

102. A method of conferring specificity for the GRP-R and/or the NMB-R on a compound comprising an optical label or metal chelator optionally complexed with a radionuclide and a GRP-R targeting peptide, comprising including in such compound a linker of the general formula:

N—O—P

wherein
N is 0, an alpha amino acid, a substituted bile acid or other linking group;
O is an alpha amino acid or a substituted bile acid; and
P is 0, an alpha amino acid, a substituted bile acid or other linking group,
   wherein at least one of N, O or P is a substituted bile acid.

103. A method of conferring specificity for the GRP-R and/or the NMB-R on a compound comprising an optical label or metal chelator optionally complexed with a radionuclide and a GRP-R targeting peptide, comprising including in such compound a linker of the general formula:

N—O—P

wherein
N is 0, an alpha amino acid, a non-alpha amino acid with a cyclic group or other linking group;
O is an alpha amino acid or a non-alpha amino acid with a cyclic group; and
P is 0, an alpha amino acid, a non-alpha amino acid with a cyclic group or other linking group,
   wherein at least one of N, O or P is a non-alpha amino acid with a cyclic group.

104. A method of improving the in vivo activity of a compound comprising an optical label or metal chelator optionally complexed with a radionuclide and a GRP-R targeting peptide, comprising including in such compound a linker of the general formula:

N—O—P

wherein
N is 0, an alpha or non-alpha amino acid or other linking group;
O is an alpha or non-alpha amino acid; and
P is 0, an alpha or non-alpha amino acid or other linking group,
   wherein at least one of N, O or P is a non-alpha amino acid.

105. A method of improving the in vivo activity of a compound comprising an optical label or metal chelator optionally complexed with a radionuclide and a GRP-R targeting peptide, comprising including in such compound a linker of the general formula:

N—O—P

wherein
N is 0, an alpha amino acid, a substituted bile acid or other linking group;
O is an alpha amino acid or a substituted bile acid; and
P is 0, an alpha amino acid, a substituted bile acid or other linking group,
   wherein at least one of N, O or P is a substituted bile acid.

106. A method of improving the in vivo stability of a compound comprising an optical label or metal chelator optionally complexed with a radionuclide and a GRP-R targeting peptide, comprising including in such compound a linker of the general formula:

N—O—P

wherein
N is 0, an alpha amino acid, a non-alpha amino acid with a cyclic group or other linking group;
O is an alpha amino acid or a non-alpha amino acid with a cyclic group; and
P is 0, an alpha amino acid, a non-alpha amino acid with a cyclic group or other linking group,
   wherein at least one of N, O or P is a non-alpha amino acid with a cyclic group.

107. A compound having the following structure:

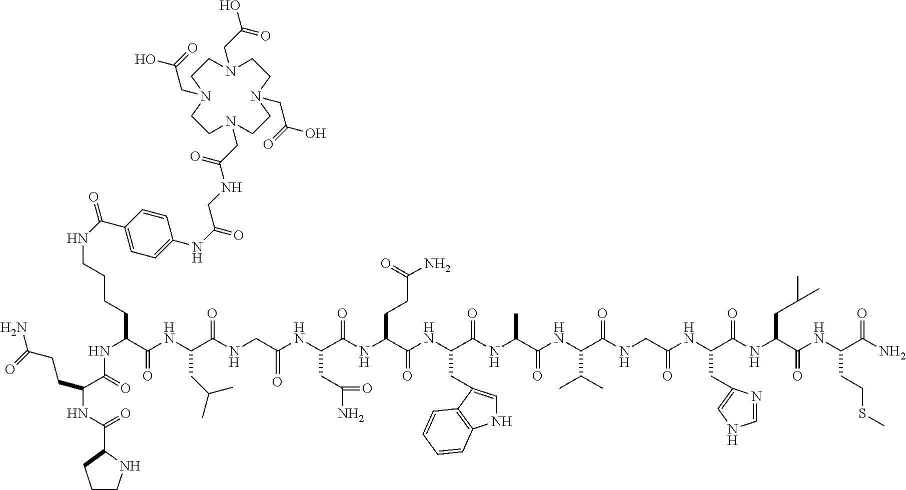 [SMILES: CSCC[C@H](NC(=O)[C@H](CC(C)C)NC(=O)[C@H](Cc1c[nH]cn1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@H](CC(N)=O)NC(=O)CNC(=O)[C@H](CC(C)C)NC(=O)[C@H](CCCCNC(=O)c1ccc(NC(=O)CNC(=O)CN2CCN(CC(=O)O)CCN(CC(=O)O)CCN(CC(=O)O)CC2)cc1)NC(=O)[C@H](CCC(N)=O)NC(=O)[C@@H]1CCCN1)C(C)C)C(N)=O]

108. A compound of the general formula:

M-N—O—P-G

wherein
M is an metal chelator of formula 8:

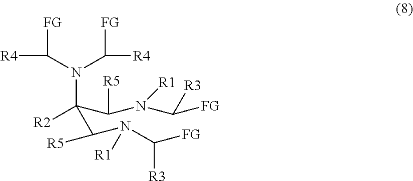

optionally complexed with a radionuclide, wherein
   R₁ is hydrogen, C₁-C₂₀ alkyl optionally substituted with one or more carboxy groups, C₃-C₁₀ cycloalkyl, C₄-C₂₀ cycloalkylalkyl, aryl, arylalkyl or the two R₁ groups, taken together, form a straight or cyclic C₂-C₁₀ alkylene group or an ortho-disubstituted arylene;
   R₂ is hydrogen, carboxy, or an optionally substituted group selected from C₁-C₂₀ alkyl, C₃-C₁₀ cycloalkyl, C₄-C₂₀ cycloalkylalkyl, aryl, arylalkyl, a group bearing an acidic moiety, and a group bearing an amino moiety, each of which may be further optionally substituted with functional groups which allow conjugation with a suitable molecule able to interact with physiological systems;
   R₃, R₄ and R₅, which can be the same or different, are hydrogen, carboxy, or an optionally substituted group selected from C₁-C₂₀ alkyl, C₃-C₁₀ cycloalkyl, C₄-C₂₀ cycloalkylalkyl, aryl, arylalkyl, a group bearing an acidic moiety and a group bearing an amino moiety, each of which may be further optionally substituted with functional groups which allow conjugation with a suitable molecule able to interact with physiological systems; and
   FG, which can be the same or different, are carboxy, —PO₃H₂ or —RP(O)OH groups, wherein R is hydrogen, or an optionally substituted group selected from C₁-C₂₀ alkyl, C₃-C₁₀ cycloalkyl, C₄-C₂₀ cycloalkylalkyl, aryl, arylalkyl, a group bearing an acidic moiety and a group bearing an amino moiety, each of which may be further optionally substituted with functional groups which allow conjugation with a suitable molecule able to interact with physiological systems;
   N is 0, an alpha amino acid, a non-alpha amino acid with a cyclic group or other linking group;
   O is an alpha amino acid or a non-alpha amino acid with a cyclic group;
   P is 0, an alpha amino acid, a non-alpha amino acid with a cyclic group, or other linking group; and
   G is a GRP receptor targeting peptide,
   wherein at least one of N, O or P is a non-alpha amino acid with a cyclic group.

109. A compound of the general formula:

M-N—O—P-G
wherein
   M is an metal chelator of formula 8:

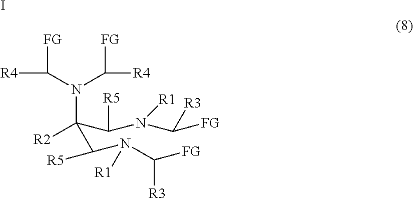

optionally complexed with a radionuclide, wherein
   R₁ is hydrogen, C₁-C₂₀ alkyl optionally substituted with one or more carboxy groups, C₃-C₁₀ cycloalkyl, C₄-C₂₀ cycloalkylalkyl, aryl, arylalkyl or the two R₁ groups, taken together, form a straight or cyclic C₂-C₁₀ alkylene group or an ortho-disubstituted arylene;
   R₂ is hydrogen, carboxy, or an optionally substituted group selected from C₁-C₂₀ alkyl, C₃-C₁₀ cycloalkyl, C₄-C₂₀ cycloalkylalkyl, aryl, arylalkyl, a group bearing an acidic moiety, and a group bearing an amino moiety, each of which may be further optionally substituted with functional groups which allow conjugation with a suitable molecule able to interact with physiological systems;
   R₃, R₄ and R₅, which can be the same or different, are hydrogen, carboxy, or an optionally substituted group selected from C₁-C₂₀ alkyl, C₃-C₁₀ cycloalkyl, C₄-C₂₀ cycloalkylalkyl, aryl, arylalkyl, a group bearing an acidic moiety and a group bearing an amino moiety, each of which may be further optionally substituted with functional groups which allow conjugation with a suitable molecule able to interact with physiological systems; and
   FG, which can be the same or different, are carboxy, —PO₃H₂ or —RP(O)OH groups, wherein R is hydrogen, or an optionally substituted group selected from C₁-C₂₀ alkyl, C₃-C₁₀ cycloalkyl, C₄-C₂₀ cycloalkylalkyl, aryl, arylalkyl, a group bearing an acidic moiety and a group bearing an amino moiety, each of which may be further optionally substituted with functional groups which allow conjugation with a suitable molecule able to interact with physiological systems;
N is 0, an alpha or non-alpha amino acid or other linking group;
O is an alpha or non-alpha amino acid; and
P is 0, an alpha or non-alpha amino acid or other linking group, and G is a GRP receptor targeting peptide,
   wherein at least one of N, O or P is a non-alpha amino acid.

110. A compound of the general formula:

M-N—O—P-G
wherein
   M is an metal chelator of formula 8:

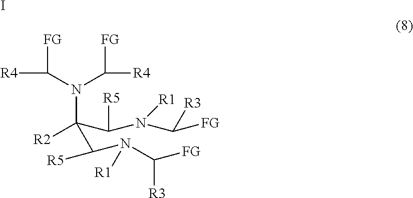

optionally complexed with a radionuclide, wherein
   R₁ is hydrogen, C₁-C₂₀ alkyl optionally substituted with one or more carboxy groups, C₃-C₁₀ cycloalkyl, C₄-C₂₀ cycloalkylalkyl, aryl, arylalkyl or the two R₁ groups, taken together, form a straight or cyclic C₂-C₁₀ alkylene group or an ortho-disubstituted arylene;
   R₂ is hydrogen, carboxy, or an optionally substituted group selected from C₁-C₂₀ alkyl, C₃-C₁₀ cycloalkyl, C₄-C₂₀ cycloalkylalkyl, aryl, arylalkyl, a group bearing an acidic moiety, and a group bearing an amino moiety, each of which may be further optionally substituted with functional groups which allow conjugation with a suitable molecule able to interact with physiological systems;
   R₃, R₄ and R₅, which can be the same or different, are hydrogen, carboxy, or an optionally substituted group selected from C₁-C₂₀ alkyl, C₃-C₁₀o cycloalkyl, C₄-C₂₀ cycloalkylalkyl, aryl, arylalkyl, a group bearing an acidic moiety and a group bearing an amino moiety, each of which may be further optionally substituted with functional groups which allow conjugation with a suitable molecule able to interact with physiological systems; and
   FG, which can be the same or different, are carboxy, —PO₃H₂ or —RP(O)OH groups, wherein R is hydrogen, or an optionally substituted group selected from C₁-C₂₀ alkyl, C₃-C₁₀ cycloalkyl, C₄-C₂₀ cycloalkylalkyl, aryl, arylalkyl, a group bearing an acidic moiety and a group bearing an amino moiety, each of which may be further optionally substituted with functional groups which allow conjugation with a suitable molecule able to interact with physiological systems;
   N is 0, an alpha amino acid, a substituted bile acid or other linking group;
   O is an alpha amino acid or a substituted bile acid; and
   P is 0, an alpha amino acid, a substituted bile acid or other linking group; and
   G is a GRP receptor targeting peptide, and
   wherein at least one of N, O or P is a substituted bile acid.

111. A compound of the general formula:

M-N—O—P-G
wherein
   M is an Aazta metal chelator or a derivative thereof optionally complexed with a radionuclide;
   N is 0, an alpha amino acid, a non-alpha amino acid with a cyclic group or other linking group;
   O is an alpha amino acid or a non-alpha amino acid with a cyclic group;
   P is 0, an alpha amino acid, a non-alpha amino acid with a cyclic group, or other linking group; and
   G is a GRP receptor targeting peptide,
wherein at least one of N, O or P is a non-alpha amino acid with a cyclic group.

112. A compound of the general formula:

M-N—O—P-G
wherein
   M is an Aazta chelator or a derivative thereof, optionally complexed with a radionuclide;
N is 0, an alpha or non-alpha amino acid or other linking group;
O is an alpha or non-alpha amino acid; and
P is 0, an alpha or non-alpha amino acid or other linking group, and G is a GRP receptor targeting peptide,
wherein at least one of N, O or P is a non-alpha amino acid.

113. A compound of the general formula:

M-N—O—P-G
wherein
   M is an Aazta metal chelator or a derivative thereof, optionally complexed with a radionuclide;
   N is 0, an alpha amino acid, a substituted bile acid or other linking group;
   O is an alpha amino acid or a substituted bile acid; and
   P is 0, an alpha amino acid, a substituted bile acid or other linking group; and
   G is a GRP receptor targeting peptide, and
wherein at least one of N, O or P is a substituted bile acid.

114. The compound of any one of embodiments 108 to 113, wherein G is an agonist or a peptide which confers agonist activity.

115. The compound of embodiment 108 or 111, wherein the non-alpha amino acid with a cyclic group is selected from the group consisting of:
4-aminobenzoic acid;
4-aminomethyl benzoic acid;
trans-4-aminomethylcyclohexane carboxylic acid;
4-(2-aminoethoxy)benzoic acid; isonipecotic acid;
2-aminomethylbenzoic acid;
4-amino-3-nitrobenzoic acid;
4-(3-carboxymethyl-2-keto-1-benzimidazolyl)-piperidine;
6-(piperazin-1-yl)-4-(3H)-quinazolinone-3-acetic acid;
)-5-amino-1,2,4,5,6,7-hexahydro-azepino[3,21-hi]indole-4-one-2-carboxylic acid;
)-4-amino-6-aza-5-oxo-9-thiabicyclo[4.3.0]nonane-7-carboxylic acid;
3-carboxymethyl-1-phenyl-1,3,8-triazaspiro[4.5]decan-4-one;
N1-piperazineacetic acid;
N-4-aminoethyl-N-1-acetic acid;
(3S)-3-amino-1-carboxymethylcaprolactam; and
,9)-6-amino-2-carboxymethyl-3,8-diazabicyclo-[4,3,0]-nonane-1,4-dione.

116. The compound of embodiment 109 or 112, wherein the non-alpha amino acid is selected from the group consisting of:
3β-amino-3,6-dioxaoctanoic acid;
N-4-aminoethyl-N-1-piperazine-acetic acid; and polyethylene glycol derivatives having the formula NH₂—(CH₂CH₂O)n-CH₂CO₂H or NH₂—(CH₂CH₂O)n-CH₂CH₂CO₂H where n=2 to 100.

117. The compound of embodiment 110 or 113, wherein the substituted bile acid is selected from the group consisting of:
3β-amino-3-deoxycholic acid;
(3β,5β)-3-aminocholan-24-oic acid;
(3β,5β,12α)-3-amino-12-hydroxycholan-24-oic acid;
(3β,5β,7α,12α)-3-amino-7,12-dihydroxycholan-24-oic acid;
Lys-(3,6,9)-trioxaundecane-1,1′-dicarbonyl-3,7-dideoxy-3-aminocholic acid);
(3β,5β,7α,12α)-3-amino-7-hydroxy-12-oxocholan-24-oic acid; and
(3β,5β,7α)-3-amino-7-hydroxycholan-24-oic acid.

118. A method of imaging comprising the steps of: administering to a patient a diagnostic imaging agent comprising the compound of any one of embodiments 109 to 113 wherein M is a metal chelator complexed with a radioactive or paramagnetic metal, and imaging said patient.

119. A method for preparing a diagnostic imaging agent comprising the step of adding to an injectable medium a substance comprising the compound of any one of embodiments 108 to 113.

120. A method of treating a patient comprising the step of administering to a patient a radiotherapeutic agent comprising the compound of any one of embodiments 108 to 113 complexed with a therapeutic radionuclide.

121. A method of preparing a radiotherapeutic agent comprising the step of adding to an injectable medium a substance comprising the compound of any one of embodiments 108 to 113.

122. The compound of embodiment 108, wherein M is Aazta, N is Gly, O is 4-aminobenzoic acid, P is 0, and G is BBN (7-14), wherein BBN(7-14) is SEQ ID NO: 1.

123. The compound of embodiment 108, wherein M is CyAazta, N is Gly, O is 4-aminobenzoic acid, P is 0, and G is BBN (7-14), wherein BBN(7-14) is SEQ ID NO: 1.

124. The compound of embodiment 110, wherein M is Aazta, N is Gly, O is (3β,5β,7α,12α)-3-amino-7,12-dihydroxycholan-24-oic acid, P is 0, and G is BBN (7-14), wherein BBN(7-14) is SEQ ID NO: 1.

125. The compound of embodiment 110, wherein M is CyAazta, N is Gly, O is (3β,5β,7α,12α)-3-amino-7,12-dihydroxycholan-24-oic acid, P is 0, and G is BBN (7-14), wherein BBN(7-14) is SEQ ID NO: 1.

126. A compound having the following structure:

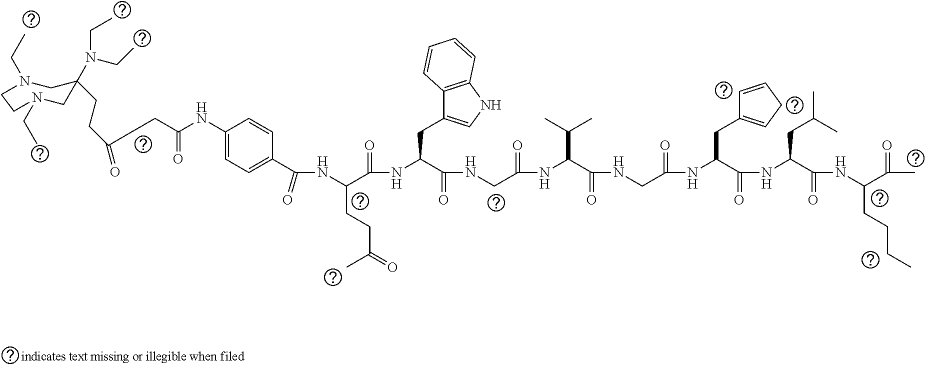

127. A compound having the following structure:

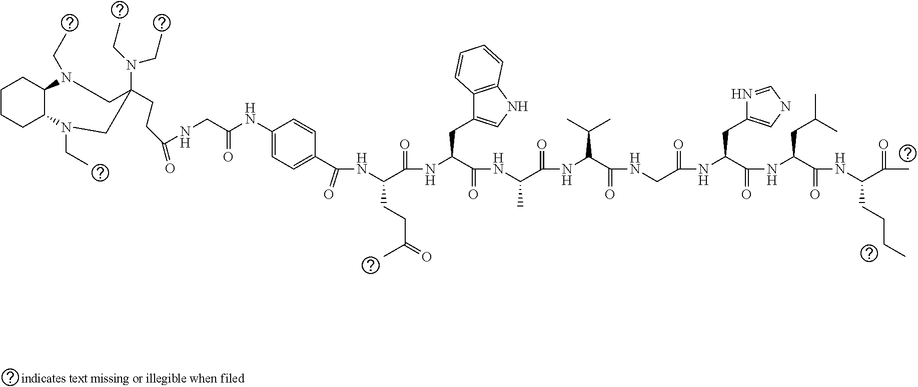

128. A method for increasing the targeting of a labeled compound to a GRP receptor expressing target tissue within a subject comprising the steps of:
administering to a subject a first dose comprising a GRP receptor targeting peptide to occupy a GRP receptor binding site in non-target tissue,
administering to said subject a second dose comprising a labeled compound of the general formula:

M-N—O—P-G
wherein
   M is an optical label or a metal chelator complexed with a radionuclide;
N is 0, an alpha or non-alpha amino acid or other linking group;
O is an alpha or non-alpha amino acid; and
P is 0, an alpha or non-alpha amino acid or other linking group, and G is a GRP receptor targeting peptide, and
wherein at least one of N, O or P is a non-alpha amino acid.

129. The method of embodiment 128, wherein the step of administering said first dose and the step of administering said second dose occur contemporaneously.

130. The method of embodiment 128, wherein the GRP receptor targeting peptide in the first dose is conjugated to a linker.

131. The method of embodiment 128, wherein the GRP receptor targeting peptide in the first dose is conjugated to a linker which is attached to one or more metal chelator.

132. The method of embodiment 131, wherein the GRP receptor targeting peptide, linker and metal chelator of said first dose are the same as the GRP receptor targeting peptide, N—O—P linker and metal chelator of the second dose.

133. The method of embodiment 128, wherein the non-alpha amino acid is selected from the group consisting of:
8-amino-3,6-dioxaoctanoic acid;
N-4-aminoethyl-N-1-piperazine-acetic acid; and polyethylene glycol derivatives having the formula NH₂—(CH₂CH₂O)n-CH₂CO₂H or NH₂—(CH₂CH₂O)n-CH₂CH₂CO₂H where n=2 to 100.

134. The method of embodiment 128, wherein the metal chelator is selected from the group consisting of DTPA, DOTA, DO3A, HP-DO3A, EDTA, TETA, EHPG, HBED, NOTA, DOTMA, TETMA, PDTA, TTHA, LICAM, MECAM, MDOTA, N,N-dimethylGly-Ser-Cys, Aazta and derivatives thereof.

135. The method of embodiment 128, wherein the labeled compound comprises a compound selected from the group consisting of:
methylglycine-Ser-Cys(Acm)-Gly-Lys-8-amino-3,6-dioxaoctanoic acid-BBN(7-14) wherein the BBN(7-14) sequence is SEQ ID NO: 1;
methylglycine-Ser-Cys(Acm)-Gly-Arg-8-amino-3,6-dioxaoctanoic acid-BBN(7-14) wherein the BBN(7-14) sequence is SEQ ID NO: 1;
methylglycine-Ser-Cys(Acm)-Gly-Asp-8-amino-3,6-dioxaoctanoic acid-BBN(7-14) wherein the BBN(7-14) sequence is SEQ ID NO: 1;
methylglycine-Ser-Cys(Acm)-Gly-Ser-8-amino-3,6-dioxaoctanoic acid-BBN(7-14) wherein the BBN(7-14) sequence is SEQ ID NO: 1;
methylglycine-Ser-Cys(Acm)-Gly-Gly-8-amino-3,6-dioxaoctanoic acid-BBN(7-14) wherein the BBN(7-14) sequence is SEQ ID NO: 1;
methylglycine-Ser-Cys(Acm)-Gly-Glu-8-amino-3,6-dioxaoctanoic acid-BBN(7-14) wherein the BBN(7-14) sequence is SEQ ID NO: 1;
methylglycine-Ser-Cys(Acm)-Gly-Dala-8-amino-3,6-dioxaoctanoic acid-BBN(7-14) wherein the BBN(7-14) sequence is SEQ ID NO: 1;
methylglycine-Ser-Cys(Acm)-Gly-8-amino-3,6-dioxaoctanoic acid-Lys-BBN(7-14) wherein the BBN(7-14) sequence is SEQ ID NO: 1;
methylglycine-Ser-Cys(Acm)-Gly-8-amino-3,6-dioxaoctanoic acid-Arg-BBN(7-14) wherein the BBN(7-14) sequence is SEQ ID NO: 1;
methylglycine-Ser-Cys(Acm)-Gly-8-amino-3,6-dioxaoctanoic acid-Asp-BBN(7-14) wherein the BBN(7-14) sequence is SEQ ID NO: 1;
methylglycine-Ser-Cys(Acm)-Gly-8-amino-3,6-dioxaoctanoic acid-Ser-BBN(7-14) wherein the BBN(7-14) sequence is SEQ ID NO: 1;
methylglycine-Ser-Cys(Acm)-Gly-8-amino-3,6-dioxaoctanoic acid-Gly-BBN(7-14) wherein the BBN(7-14) sequence is SEQ ID NO: 1;
methylglycine-Ser-Cys(Acm)-Gly-8-amino-3,6-dioxaoctanoic acid-Glu-BBN(7-14) wherein the BBN(7-14) sequence is SEQ ID NO: 1;
methylglycine-Ser-Cys(Acm)-Gly-8-amino-3,6-dioxaoctanoic acid-Dala-BBN(7-14) wherein the BBN(7-14) sequence is SEQ ID NO: 1;
methylglycine-Ser-Cys(Acm)-Gly-8-amino-3,6-dioxaoctanoic acid-8-amino-3,6-dioxaoctanoic acid-BBN(7-14) wherein the BBN(7-14) sequence is SEQ ID NO: 1;
methylglycine-Ser-Cys(Acm)-Gly-8-amino-3,6-dioxaoctanoic acid-2,3-diaminopropionic acid-BBN(7-14) wherein the BBN(7-14) sequence is SEQ ID NO: 1;
methylglycine-Ser-Cys(Acm)-Gly-8-amino-3,6-dioxaoctanoic acid-BBN(7-14) wherein the BBN(7-14) sequence is SEQ ID NO: 1;
methylglycine-Ser-Cys(Acm)-Gly-2,3-diaminopropionic acid-8-amino-3,6-dioxaoctanoic acid-BBN(7-14) wherein the BBN(7-14) sequence is SEQ ID NO: 1;
methylglycine-Ser-Cys(Acm)-Gly-Asp-8-amino-3,6-dioxaoctanoic acid-Gly-BBN(7-14) wherein the BBN(7-14) sequence is SEQ ID NO: 1;
methylglycine-Ser-Cys(Acm)-Gly-8-amino-3,6-dioxaoctanoic acid-Asp-Gly-BBN(7-14) wherein the BBN(7-14) sequence is SEQ ID NO: 1;
methylglycine-Ser-Cys(Acm)-Gly-8-amino-3,6-dioxaoctanoic acid-Ser-Gly-BBN(7-14) wherein the BBN(7-14) sequence is SEQ ID NO: 1;
methylglycine-Ser-Cys(Acm)-Gly-8-amino-3,6-dioxaoctanoic acid-Arg-Gly-BBN(7-14) wherein the BBN(7-14) sequence is SEQ ID NO: 1;
methylglycine-Ser-Cys(Acm)-Gly-8-amino-3,6-dioxaoctanoic acid-8-amino-3,6-dioxaoctanoic acid-Gly-BBN(7-14) wherein the BBN(7-14) sequence is SEQ ID NO: 1;
methylglycine-Ser-Cys(Acm)-Gly-8-amino-3,6-dioxaoctanoic acid-2,3-diaminopropionic acid-Gly-BBN(7-14) wherein the BBN(7-14) sequence is SEQ ID NO: 1;
methylglycine-Ser-Cys(Acm)-Gly-8-amino-3,6-dioxaoctanoic acid-Lys-Gly-BBN(7-14) wherein the BBN(7-14) sequence is SEQ ID NO: 1;
methylglycine-Ser-Cys(Acm)-Gly-2,3-diaminopropionic acid-8-amino-3,6-dioxaoctanoic acid-Gly-BBN(7-14) wherein the BBN(7-14) sequence is SEQ ID NO: 1;
methylglycine-Ser-Cys(Acm)-Gly-N-4-aminoethyl-N-1-piperazineacetic acid-Asp-BBN(7-14) wherein the BBN(7-14) sequence is SEQ ID NO: 1;
methylglycine-Ser-Cys(Acm)-Gly-N-4-aminoethyl-N-1-piperazineacetic acid-Ser-BBN(7-14) wherein the BBN(7-14) sequence is SEQ ID NO: 1;
methylglycine-Ser-Cys(Acm)-Gly-N-4-aminoethyl-N-1-piperazineacetic acid-Arg-BBN(7-14) wherein the BBN(7-14) sequence is SEQ ID NO: 1;
methylglycine-Ser-Cys(Acm)-Gly-N-4-aminoethyl-N-1-piperazineacetic acid-8-amino-3,6-dioxaoctanoic acid-BBN(7-14) wherein the BBN(7-14) sequence is SEQ ID NO: 1;
methylglycine-Ser-Cys(Acm)-Gly-N-4-aminoethyl-N-1-piperazineacetic acid-2,3-diaminopropionic acid-BBN(7-14) wherein the BBN(7-14) sequence is SEQ ID NO: 1;
methylglycine-Ser-Cys(Acm)-Gly-N-4-aminoethyl-N-1-piperazineacetic acid-Lys-BBN(7-14) wherein the BBN(7-14) sequence is SEQ ID NO: 1;
methylglycine-Ser-Cys(Acm)-Gly-N-1-piperazineacetic acid-Asp-BBN(7-14) wherein the BBN(7-14) sequence is SEQ ID NO: 1;
methylglycine-Ser-Cys(Acm)-Gly-N-1-piperazineacetic acid-Ser-BBN(7-14) wherein the BBN(7-14) sequence is SEQ ID NO: 1;
methylglycine-Ser-Cys(Acm)-Gly-N-1-piperazineacetic acid-Arg-BBN(7-14) wherein the BBN(7-14) sequence is SEQ ID NO: 1;
methylglycine-Ser-Cys(Acm)-Gly-N-1-piperazineacetic acid-8-amino-3,6-dioxaoctanoic acid-BBN(7-14) wherein the BBN(7-14) sequence is SEQ ID NO: 1;
methylglycine-Ser-Cys(Acm)-Gly-N-1-piperazineacetic acid-2,3-diaminopropionic acid BBN(7-14) wherein the BBN(7-14) sequence is SEQ ID NO: 1;
methylglycine-Ser-Cys(Acm)-Gly-N-1-piperazineacetic acid-Lys-BBN(7-14) wherein the BBN(7-14) sequence is SEQ ID NO: 1;
methylglycine-Ser-Cys(Acm)-Gly-4-Hydroxyproline-8-amino-3,6-dioxaoctanoic acid-BBN(7-14) wherein the BBN(7-14) sequence is SEQ ID NO: 1;
methylglycine-Ser-Cys(Acm)-Gly-4-aminoproline-8-amino-3,6-dioxaoctanoic acid-BBN(7-14) wherein the BBN(7-14) sequence is SEQ ID NO: 1;
methylglycine-Ser-Cys(Acm)-Gly-Lys-8-amino-3,6-dioxaoctanoic acid-Gly-BBN(7-14) wherein the BBN(7-14) sequence is SEQ ID NO: 1;
methylglycine-Ser-Cys(Acm)-Gly-Arg-8-amino-3,6-dioxaoctanoic acid-Gly-BBN(7-14) wherein the BBN(7-14) sequence is SEQ ID NO: 1;
methylglycine-Ser-Cys(Acm)-Gly-Ser-8-amino-3,6-dioxaoctanoic acid-Gly-BBN(7-14) wherein the BBN(7-14) sequence is SEQ ID NO: 1;
methylglycine-Ser-Cys(Acm)-Gly-Asp-8-amino-3,6-dioxaoctanoic acid-Gly-BBN(7-14) wherein the BBN(7-14) sequence is SEQ ID NO: 1;
methylglycine-Ser-Cys(Acm)-Gly-8-amino-3,6-dioxaoctanoic acid-Asp-Gly-BBN(7-14) wherein the BBN(7-14) sequence is SEQ ID NO: 1;
methylglycine-Ser-Cys(Acm)-Gly-8-amino-3,6-dioxaoctanoic acid-Ser-Gly-BBN(7-14) wherein the BBN(7-14) sequence is SEQ ID NO: 1;
methylglycine-Ser-Cys(Acm)-Gly-8-amino-3,6-dioxaoctanoic acid-Arg-Gly-BBN(7-14) wherein the BBN(7-14) sequence is SEQ ID NO: 1;
methylglycine-Ser-Cys(Acm)-Gly-8-amino-3,6-dioxaoctanoic acid-8-amino-3,6-dioxaoctanoic acid-Gly-BBN(7-14) wherein the BBN(7-14) sequence is SEQ ID NO: 1;
methylglycine-Ser-Cys(Acm)-Gly-8-amino-3,6-dioxaoctanoic acid-2,3-diaminopropionic acid-Gly-BBN(7-14) wherein the BBN(7-14) sequence is SEQ ID NO: 1;
methylglycine-Ser-Cys(Acm)-Gly-8-amino-3,6-dioxaoctanoic acid-Lys-Gly-BBN(7-14) wherein the BBN(7-14) sequence is SEQ ID NO: 1; and
methylglycine-Ser-Cys(Acm)-Gly-2,3-diaminopropionic acid-8-amino-3,6-dioxaoctanoic acid-Gly-BBN(7-14) wherein the BBN(7-14) sequence is SEQ ID NO: 1.

136. The method of embodiment 128, wherein the labeled compound comprises a compound selected from the group consisting of:

-monoamide-8-amino-3,6-dioxaoctanoic acid-diaminopropionic acid-BBN(7-14) wherein the BBN(7-14) sequence is SEQ ID NO: 1;
-monoamide-8-amino-3,6-dioxaoctanoic acid-biphenylalanine-BBN(7-14) wherein the BBN(7-14) sequence is SEQ ID NO: 1;
-monoamide-8-amino-3,6-dioxaoctanoic acid-diphenylalanine-BBN(7-14) wherein the BBN(7-14) sequence is SEQ ID NO: 1;
-monoamide-8-amino-3,6-dioxaoctanoic acid-4-benzoylphenylalanine-BBN(7-14) wherein the BBN(7-14) sequence is SEQ ID NO: 1;
-monoamide-5-aminopentanoic acid-8-amino-3,6-dioxaoctanoic acid-BBN(7-14) wherein the BBN(7-14) sequence is SEQ ID NO: 1;
-monoamide-8-amino-3,6-dioxaoctanoic acid-D-phenylalanine-BBN(7-14) wherein the BBN(7-14) sequence is SEQ ID NO: 1;
-monoamide-8-aminooctanoic acid-8-amino-3,6-dioxaoctanoic acid-BBN(7-14) wherein the BBN(7-14) sequence is SEQ ID NO: 1;
-monoamide-E(G8-amino-3,6-dioxaoctanoic acid-8-amino-3,6-dioxaoctanoic acid QWAVGHLM-NH₂)-8-aminooctanoic acid-8-aminooctanoic acid-BBN(7-14) wherein the BBN(7-14) sequence is SEQ ID NO: 1; and
-monoamide-E(G-Aoa-Aoa-QWAVGHLM-NH₂)-8-aminooctanoic acid-8-aminooctanoic acid-BBN(7-14) wherein the BBN(7-14) sequence is SEQ ID NO: 1.

137. A method for increasing the targeting of a labeled compound to a GRP receptor expressing target tissue within a subject comprising the steps of:
administering to a subject a first dose comprising a GRP receptor targeting peptide to occupy GRP receptor binding sites in non-target tissues,
administering to said subject a second dose comprising a labeled compound of the general formula:

M-N—O—P-G
wherein
M is an optical label or a metal chelator complexed with a radionuclide;
N is 0, an alpha amino acid, a substituted bile acid or other linking group;
O is an alpha amino acid or a substituted bile acid;
P is 0, an alpha amino acid, a substituted bile acid or other linking group;
G is a GRP receptor targeting peptide, and
wherein at least one of N, O or P is a substituted bile acid.

138. The method of embodiment 137, wherein the step of administering said first dose and the step of administering said second dose occur contemporaneously.

139. The method of embodiment 137, wherein the GRP receptor targeting peptide in the first dose is conjugated to a linker.

140. The method of embodiment 137 wherein the GRP receptor targeting peptide in the first dose is conjugated to a linker which is attached to one or more metal chelator.

141. The method embodiment 140 wherein the GRP receptor targeting peptide, linker and metal chelator of said first dose are the same as the GRP receptor targeting peptide, N—O—P linker and metal chelator of the second dose.

142. The method of embodiment 137, wherein the substituted bile acid is selected from the group consisting of:
3β-amino-3-deoxycholic acid;
(3β,5β)-3-aminocholan-24-oic acid;
(3β,5β,12α)-3-amino-12-hydroxycholan-24-oic acid;
(3β,5β,7α,12α)-3-amino-7,12-dihydroxycholan-24-oic acid;
Lys-(3,6,9)-trioxaundecane-1,1′-dicarbonyl-3,7-dideoxy-3-aminocholic acid);
(3β,5β,7α,12α)-3-amino-7-hydroxy-12-oxocholan-24-oic acid; and
(3β,5β,7α)-3-amino-7-hydroxycholan-24-oic acid.

143. The method of embodiment 137, wherein the metal chelator is selected from the group consisting of: DTPA, DOTA, DO3A, HPDO3A, EDTA, TETA, CMDOTA, N,N-dimethylGly-Ser-Cys, Aazta and derivatives thereof.

144. The method of embodiment 137, wherein the labeled compound comprises a compound selected from the group consisting of:

-monoamide-Gly-(3β,5β)-3-aminocholan-24-oic acid-BBN(7-14) wherein the BBN(7-14) sequence is SEQ ID NO: 1;
-monoamide-Gly-(3β,5β,12α)-3-amino-12-hydroxycholan-24-oic acid-BBN(7-14) wherein the BBN(7-14) sequence is SEQ ID NO: 1;
-monoamide-Gly-(3β,5β,7α,12α)-3-amino-7,12-dihydroxycholan-24-oic acid-BBN(7-14) wherein the BBN(7-14) sequence is SEQ ID NO: 1;
-monoamide-Gly-Lys-(3,6,9)-trioxaundecane-1,1′-dicarbonyl-3,7-dideoxy-3-aminocholic acid)-Arg-BBN(7-14) wherein the BBN(7-14) sequence is SEQ ID NO: 1;
,7α,12α)-3-amino-7,12-dihydroxycholan-24-oic acid-3,6,9-trioxaundecane-1,1′-dicarbonyl Lys(DO3A-monoamide-Gly)-Arg-BBN(7-14) wherein the BBN(7-14) sequence is SEQ ID NO: 1;
   -monoamide-Gly-(3β,5β,7α,12α)-3-amino-12-oxocholan-24-oic acid-BBN(7-14) wherein the BBN(7-14) sequence is SEQ ID NO: 1;
   -monoamide-1-amino-3,6-dioxaoctanoic acid-(3β,5β,7α,12α)-3-amino-7,12-dihydroxycholan-24-oic acid-BBN(7-14) wherein the BBN(7-14) sequence is SEQ ID NO: 1;
   -monoamide-Gly-(3β,5β,7α,12α)-3-amino-7,12-dihydroxycholan-24-oic acid-QWAVaHLM-NH₂ wherein QWAVaHLM-NH₂ is SEQ ID NO: 14;
   -monoamide-Gly-(3β,5β,7α,12α)-3-amino-7,12-dihydroxycholan-24-oic acid-f-QWAVGHLM-NH: wherein QWAVGHLM-NH₂ is SEQ ID NO: 1;
   -monoamide-Gly-(3β,5β,7α,12α)-3-amino-7,12-dihydroxycholan-24-oic acid-f-WAVGHLL-NH₂ wherein WAVGHLL-NH₂ is SEQ ID NO: 8;
   -monoamide-Gly-(3β,5β,7α,12α)-3-amino-7,12-dihydroxycholan-24-oic acid-f-QWAVGHL-NH-pentyl wherein QWAVGHL-NH-pentyl is SEQ ID NO: 6;
   -monoamide-Gly-(3β,5β,7α,12α)-3-amino-7,12-dihydroxycholan-24-oic acid-γ-QWAV-Bala-H-F-Nle-NH₂ wherein QWAV-Bala-H-F-Nle-NH₂ is SEQ ID NO: 9;
   -monoamide-Gly-(3β,5β,7α,12α)-3-amino-7,12-dihydroxycholan-24-oic acid-f-QWAV-Bala-H-F-Nle-NH₂ wherein QWAV-Bala-H-F-Nle-NH₂ is SEQ ID NO: 9;
   -monoamide-Gly-(3β,5β,7α,12α)-3-amino-7,12-dihydroxycholan-24-oic acid-QWAVGHFL-NH₂ wherein QWAVGHFL-NH₂ is SEQ ID NO: 22;
   -monoamide-Gly-(3β,5β,7α,12α)-3-amino-7,12-dihydroxycholan-24-oic acid-QWAVGNMeH-L-M-NH₂ wherein QWAVGNMeH-L-M-NH₂ is SEQ ID NO: 15;
   -monoamide-Gly-(3β,5β,7α,12α)-3-amino-7,12-dihydroxycholan-24-oic acid-LWAVGSF-M-NH₂ wherein LWAVGSF-M-NH₂ is SEQ ID NO: 11;
   -monoamide-Gly-(3β,5β,7α,12α)-3-amino-7,12-dihydroxycholan-24-oic acid-HWAVGHLM-NH₂ wherein HWAVGHLM-NH₂ is SEQ ID NO: 12;
   -monoamide-Gly-(3β,5β,7α,12α)-3-amino-7,12-dihydroxycholan-24-oic acid-LWAGHFM-NH₂ wherein LWAGHFM-NH₂ is SEQ ID NO: 20;
   -monoamide-Gly-(3β,5β,7α,12α)-3-amino-7,12-dihydroxycholan-24-oic acid-QWAVGHFM-NH₂ wherein QWAVGHFM-NH₂ is SEQ ID NO: 13;
   -monoamide-Gly-(3β,5β,7α,12α)-3-amino-7,12-dihydroxycholan-24-oic acid-QRLGNQWAVGHLM-NH₂ wherein QRLGNQWAVGHLM-NH₂ is SEQ ID NO: 3;
   -monoamide-Gly-(3β,5β,7α,12α)-3-amino-7,12-dihydroxycholan-24-oic acid-QRYGNQWAVGHLM-NH₂ wherein QRYGNQWAVGHLM-NH₂ is SEQ ID NO: 4;
   -monoamide-Gly-(3β,5β,7α,12α)-3-amino-7,12-dihydroxycholan-24-oic acid-QKYGNQWAVGHLM-NH₂ wherein QKYGNQWAVGHLM-NH₂ is SEQ ID NO: 5;
   Lys (DO3A-monoamide)-Gly-(3β,5β,7α,12α)-3-amino-7,12-dihydroxycholan-24-oic acid-LGNQWAVGHLM-NH₂ wherein LGNQWAVGHLM-NH₂ is SEQ ID NO: 18;
   -monoamide-Gly-3-amino-3-deoxycholic acid-QRLGNQWAVGHLM-NH₂ wherein QRLGNQWAVGHLM-NH₂ is SEQ ID NO: 3;
   -monoamide-Gly-3-amino-3-deoxycholic acid-QRYGNQWAVGHLM-NH₂ wherein QRYGNQWAVGHLM-NH₂ is SEQ ID NO: 4;
   -monoamide-Gly-3-amino-3-deoxycholic acid-QKYGNQWAVGHLM-NH₂ wherein QKYGNQWAVGHLM-NH₂ is SEQ ID NO: 5;
   Lys(DO3A-monoamide-G-3-amino-3-deoxycholic acid)-LGNQWAVGHLM-NH₂ wherein LGNQWAVGHLM-NH₂ is SEQ ID NO: 18;
   Gly-(3β,5β,7α,12α)-3-amino-7,12-dihydroxycholan-24-oic acid-BBN(7-14) wherein the BBN(7-14 sequence is SEQ ID NO: 1; and
   ta-Gly-(3β,5β,7α,12α)-3-amino-7,12-dihydroxycholan-24-oic acid-BBN(7-14) wherein the BBN(7-14) sequence is SEQ ID NO: 1.

145. The method of embodiment 137 wherein said labeled compound comprises DO3A-monoamide-Gly-(3β,5β,7α,12α)-3-amino-7,12-dihydroxycholan-24-oic acid-BBN(7-14) wherein the BBN(7-14) sequence is SEQ ID NO: 1.

146. A method for increasing the targeting of a labeled compound to a GRP receptor expressing target tissue within a subject comprising the steps of:
administering to a subject a first dose comprising a GRP receptor targeting peptide to occupy a GRP receptor binding site in non-target tissue,
administering to said subject a second dose comprising a labeled compound of the general formula:

M-N—O—P-G
wherein
   M is an optical label or a metal chelator complexed with a radionuclide;
   N is 0, an alpha amino acid, a non-alpha amino acid with a cyclic group or other linking group;
   O is an alpha amino acid or a non-alpha amino acid with a cyclic group;
   P is 0, an alpha amino acid, a non-alpha amino acid with a cyclic group, or other linking group; and
   G is a GRP receptor targeting peptide,
wherein at least one of N, O or P is a non-alpha amino acid with a cyclic group.

147. The method of embodiment 146, wherein the step of administering said first dose and the step of administering said second dose occur contemporaneously.

148. The method of embodiment 146, wherein the GRP receptor targeting peptide in the first dose is conjugated to a linker.

149. The method of embodiment 146, wherein the GRP receptor targeting peptide in the first dose is conjugated to a linker which is attached to one or more metal chelator.

150. The method of embodiment 149, wherein said GRP receptor peptide, linker and metal chelator of said first dose are the same as the GRP receptor targeting peptide, N—O—P linker and metal chelator of the second dose.

151. The method of embodiment 146, wherein the non-alpha amino acid with a cyclic group is selected from the group consisting of:
[symbol] obenzoic acid;
omethyl benzoic acid;
-aminomethylcyclohexane carboxylic acid;
minoethoxy)benzoic acid;
ecotic acid;
omethylbenzoic acid;
o-3-nitrobenzoic acid;
arboxymethyl-2-keto-1-benzimidazolyl)-piperidine;
erazin-1-yl)-4-(3H)-quinazolinone-3-acetic acid;
)-5-amino-1,2,4,5,6,7-hexahydro-azepino[3,2,1-hi]indole-4-one-2-carboxylic acid;
)-4-amino-6-aza-5-oxo-9-thiabicyclo[4.3.0]nonane-7-carboxylic acid;
oxymethyl-1-phenyl-1,3,8-triazaspiro[4.5]decan-4-one;
erazineacetic acid;
minoethyl-N-1-acetic acid;
-amino-1-carboxymethylcaprolactam; and
,9)-6-amino-2-carboxymethyl-3,8-diazabicyclo-[4,3,O]-nonane-1,4-dione.

152. The method of embodiment 146 wherein the metal chelator is selected from the group consisting of: DTPA, DOTA, DO3A, HPDO3A, EDTA, TETA, N,N-dimethylGly-Ser-Cys, Aazta and derivatives thereof.

153. The method of embodiment 146, wherein the labeled compound comprises a compound selected from the group consisting of

[symbol]

-monoamide-Gly-4-aminobenzoic acid-BBN(7-14) wherein the BBN(7-14) sequence is SEQ ID NO: 1;
-monoamide-4-aminomethyl benzoic acid-BBN(7-14) wherein the BBN(7-14) sequence is SEQ ID NO: 1;
-monoamide-trans-4-aminomethylcyclohexyl carboxylic acid-BBN(7-14) wherein the BBN(7-14) sequence is SEQ ID NO: 1;
-monoamide-4-(2-aminoethoxy)benzoic acid-BBN(7-14) wherein the BBN(7-14) sequence is SEQ ID NO: 1;
-monoamide-Gly-isonipecotic acid-BBN(7-14) wherein the BBN(7-14) sequence is SEQ ID NO: 1;
-monoamide-2-aminomethylbenzoic acid-BBN(7-14) wherein the BBN(7-14) sequence is SEQ ID NO: 1;
-monoamide-4-aminomethyl-3-nitrobenzoic acid-BBN(7-14) wherein the BBN(7-14) sequence is SEQ ID NO: 1;
-monoamide-8-amino-3,6-dioxaoctanoic acid-1-naphthylalanine-BBN(7-14) wherein the BBN(7-14) sequence is SEQ ID NO: 1;
-monoamide-4-(3-carboxymethyl-2-keto-1-benzimidazolyl-piperidine-BBN(7-14) wherein the BBN(7-14) sequence is SEQ ID NO: 1;
-monoamide-6-(piperazin-1-yl)-4-(3H)-quinazolinone-3-acetic acid-BBN(7-14) wherein the BBN(7-14) sequence is SEQ ID NO: 1;
-monoamide-(2S,5S)-5-amino-1,2,4,5,6,7-hexahydro-azepino[3,2,1-hi]indole-4-one-2-carboxylic acid-BBN(7-14) wherein the BBN(7-14) sequence is SEQ ID NO: 1;
-monoamide-(4S,7R)-4-amino-6-aza-5-oxo-9-thiabicyclo[4.3.0]nonane-7-carboxylic acid-BBN(7-14) wherein the BBN(7-14) sequence is SEQ ID NO: 1;
-monoamide-N,N-dimethylglycine-BBN(7-14) wherein the BBN(7-14) sequence is SEQ ID NO: 1;
-monoamide-3-carboxymethyl-1-phenyl-1,3,8-triazaspiro[4.5]decan-4-one-BBN(7-14) wherein the BBN(7-14) sequence is SEQ ID NO: 1;
-monoamide-N-1-piperazineacetic acid-BBN(7-14) wherein the BBN(7-14) sequence is SEQ ID NO: 1;
-monoamide-N4-aminoethyl-N-1-piperazineacetic acid-BBN(7-14) wherein the BBN(7-14) sequence is SEQ ID NO: 1;
-monoamide-(3S)-3-amino-1-carboxymethylcaprolactam-BBN(7-14) wherein the BBN(7-14) sequence is SEQ ID NO: 1;
-monoamide-(2S,6S,9)-6-amino-2-carboxymethyl-3,8-diazabicyclo-[4,3,O]-nonane-1,4-dione-BBN(7-14) wherein the BBN(7-14) sequence is SEQ ID NO: 1;
-monoamide-5-aminopentanoic acid-trans-4-aminomethylcyclohexane-1-carboxylic acid-BBN(7-14) wherein the BBN(7-14) sequence is SEQ ID NO: 1;
-monoamide-trans-4-aminomethylcyclohexane-1-carboxylic acid-D-phenylalanine-BBN(7-14) wherein the BBN(7-14) sequence is SEQ ID NO: 1;
-monoamide-4-aminomethylbenzoic acid-8-amino-3,6-dioxaoctanoic acid-BBN(7-14) wherein the BBN(7-14) sequence is SEQ ID NO: 1;
-monoamide-4-benzoyl-(L)-phenylalanine-trans-4-aminomethylcyclohexane-1-carboxylic acid-BBN(7-14) wherein the BBN(7-14) sequence is SEQ ID NO: 1;
-monoamide-trans-4-aminomethylcyclohexane-1-carboxylic acid-Arg-BBN(7-14) wherein the BBN(7-14) sequence is SEQ ID NO: 1;
-monoamide-trans-4-aminomethylcyclohexane-1-carboxylic acid-Lys-BBN(7-14) wherein the BBN(7-14) sequence is SEQ ID NO: 1;
-monoamide-trans-4-aminomethylcyclohexane-1-carboxylic acid-diphenylalanine-BBN(7-14) wherein the BBN(7-14) sequence is SEQ ID NO: 1;
-monoamide-trans-4-aminomethylcyclohexane-1-carboxylic acid-1-naphthylalanine-BBN(7-14) wherein the BBN(7-14) sequence is SEQ ID NO: 1;
-monoamide-trans-4-aminomethylcyclohexane-1-carboxylic acid-8-amino-3,6-dioxaoctanoic acid-BBN(7-14) wherein the BBN(7-14) sequence is SEQ ID NO: 1;
-monoamide-trans-4-aminomethylcyclohexane-1-carboxylic acid-Ser-BBN(7-14) wherein the BBN(7-14) sequence is SEQ ID NO: 1;
-monoamide-trans-4-aminomethylcyclohexane-1-carboxylic acid-2,3-diaminopropionic acid-BBN(7-14) wherein the BBN(7-14) sequence is SEQ ID NO: 1;
-monoamide-trans-4-aminomethylcyclohexane-1-carboxylic acid-biphenylalanine-BBN(7-14) wherein the BBN(7-14) sequence is SEQ ID NO: 1;
-monoamide-trans-4-aminomethylcyclohexane-1-carboxylic acid-(2S,5S)-5-amino-1,2,4,5,6,7-hexahydro-azepino[3,21-hi]indole-4-one-2-carboxylic acid-BBN(7-14) wherein the BBN(7-14) sequence is SEQ ID NO: 1;
-monoamide-trans-4-aminomethylcyclohexane-1-carboxylic acid-trans-4-aminomethylcyclohexane-1-carboxylic acid-BBN(7-14) wherein the BBN(7-14) sequence is SEQ ID NO: 1;
L-monoamide-8-amino-3,6-dioxaoctanoic acid-phenylalanine-BBN(7-14) wherein the BBN(7-14) sequence is SEQ ID NO: 1;
-monoamide-trans-4-aminomethylcyclohexane-1-carboxylic acid-phenylalanine-BBN(7-14) wherein the BBN(7-14) sequence is SEQ ID NO: 1;
-monoamide-8-aminooctanoic acid-trans-4-aminomethylcyclohexane-1-carboxylic acid-BBN(7-14) wherein the BBN(7-14) sequence is SEQ ID NO: 1;
-monoamide-4′-aminomethyl-biphenyl-1-carboxylic acid-BBN(7-14) wherein the BBN(7-14) sequence is SEQ ID NO: 1;
-monoamide-3′-aminomethyl-biphenyl-3-carboxylic acid-BBN(7-14) wherein the BBN(7-14) sequence is SEQ ID NO: 1;
OTA-Gly-4-aminobenzoic acid-BBN(7-14) wherein the BBN(7-14) sequence is SEQ ID NO: 1;
-monoamide-4-aminomethylphenoxyacetic acid-BBN(7-14) wherein the BBN(7-14) sequence is SEQ ID NO: 1;
-monoamide-Gly-4-aminophenylacetic acid-BBN(7-14) wherein the BBN(7-14) sequence is SEQ ID NO: 1;
OTA-4-phenoxy-BBN(7-14) wherein the BBN(7-14) sequence is SEQ ID NO: 1;
-monoamide-3-aminomethylbenzoic acid-BBN(7-14) wherein the BBN(7-14) sequence is SEQ ID NO: 1;
-monoamide-4-aminomethylphenylacetic acid-BBN(7-14) wherein the BBN(7-14) sequence is SEQ ID NO: 1;
-monoamide-4-aminomethyl-3-methoxybenzoic acid-BBN(7-14) wherein the BBN(7-14) sequence is SEQ ID NO: 1;
Gly-4-aminobenzoic acid-BBN(7-14) wherein the BBN(7-14) sequence is SEQ ID NO: 1;
-monoamide-Gly-4-hydrazinobenzoyl-BBN(7-14) wherein the BBN(7-14) sequence is SEQ ID NO: 1;
-monoamide-4-aminobenzoic acid-BBN(7-14) wherein the BBN(7-14) sequence is SEQ ID NO: 1;
-monoamide-4-aminobenzoic acid-Gly-BBN(7-14) wherein the BBN(7-14) sequence is SEQ ID NO: 1;
-monoamide-Gly-6-Aminonicotinic acid-BBN(7-14) wherein the BBN(7-14) sequence is SEQ ID NO: 1;
-monoamide-Gly-4′-Amino-2′-methyl biphenyl-4-carboxylic acid-BBN(7-14) wherein the BBN(7-14) sequence is SEQ ID NO: 1;
-monoamide-Gly-3′-Aminobiphenyl-3-carboxylic acid-BBN(7-14) wherein the BBN(7-14) sequence is SEQ ID NO: 1;
-monoamide-Gly-1,2-diaminoethyl-Terephthalic acid-BBN(7-14) wherein the BBN(7-14) sequence is SEQ ID NO: 1;
-monoamide-Gly-Gly-4-aminobenzoic acid-BBN(7-14) wherein the BBN(7-14) sequence is SEQ ID NO: 1;
-monoamide-G-4-aminobenzoic acid-EWAVGHLM-NH₂ wherein EWAVGHLM-NH₂ is SEQ ID NO: 2;
-monoamide-G-4-aminobenzoic acid-QWAVGHLM-OH wherein QWAVGHLM-OH is SEQ ID NO: 1;
-monoamide-G-4-aminobenzoic acid-(D)-Phe-BBN(7-14) wherein the BBN(7-14) sequence is SEQ ID NO: 1;
-monoamide-G-4-aminobenzoic acid-QRLGNQWAVGHLM-NH₂ wherein QRLGNQWAVGHLM-NH₂ is SEQ ID NO: 3;
-monoamide-G-4-aminobenzoic acid-QRYGNQWAVGHLM-NH₂ wherein QRYGNQWAVGHLM-NH₂ is SEQ ID NO: 4;
-monoamide-G-4-aminobenzoic acid-QKYGNQWAVGHLM-NH₂ wherein QKYGNQWAVGHLM-NH₂ is SEQ ID NO: 5;
-monoamide-G-4-aminobenzoic acid-(D)-Phe-QWAVGHL-NH-Pentyl wherein QWAVGHL-NH-Pentyl is SEQ ID NO: 6;
-monoamide-G-4-aminobenzoic acid-QWSVaHLM-NH₂ wherein QWSVaHLM-NH₂ is SEQ ID NO: 7;
-monoamide-G-4-aminobenzoic acid-(D)-Phe-QWAVGHLL-NH₂ QWAVGHLL-NH₂ is SEQ ID NO: 8;
L-monoamide-G-4-aminobenzoic acid-(D)-Tyr-QWAV-Bala-HF-Nle-NH₂ wherein QWAV-Bala-HF-Nle-NH₂ is SEQ ID NO: 9;
L-monoamide-G-4-aminobenzoic acid-Phe-QWAV-Bala-HF-Nle-NH₂ wherein QWAV-Bala-HF-Nle-NH₂ is SEQ ID NO: 9;
-monoamide-G-4-aminobenzoic acid-QWAGHFL-NH₂ wherein QWAGHFL-NH₂ is SEQ ID NO: 10;
-monoamide-G-4-aminobenzoic acid-LWAVGSFM-NH₂ wherein LWAVGSFM-NH₂ is SEQ ID NO: 11;
-monoamide-G-4-aminobenzoic acid-HWAVGHLM-NH₂ wherein HWAVGHLM-NH₂ is SEQ ID NO: 12;
-monoamide-G-4-aminobenzoic acid-LWAVGSFM-NH₂ wherein LWAVGSFM-NH₂ is SEQ ID NO: 11;
-monoamide-G-4-aminobenzoic acid-QWAVGHFM-NH₂ wherein QWAVGHFM-NH₂ is SEQ ID NO: 13;
-monoamide-Gly-3-aminobenzoic acid-BBN(7-14) wherein the BBN(7-14) sequence is SEQ ID NO: 1;
-monoamide-Gly-6-aminonaphthoic acid-BBN(7-14) wherein the BBN(7-14) sequence is SEQ ID NO: 1;
-monoamide-Gly-4-methylaminobenzoic acid-BBN(7-14) wherein the BBN(7-14) sequence is SEQ ID NO: 1;
m10d2a-Gly-4-aminobenzoic acid-BBN(7-14) wherein the BBN(7-14) sequence is SEQ ID NO: 1;
imethylglycine-Ser-Cys(Acm)-Gly-Gly-4-aminobenzoic acid-BBN(7-14) wherein the BBN(7-14) sequence is SEQ ID NO: 1;
imethylglycine-Ser-Cys(Acm)-Gly-Gly-3-amino-3-deoxycholic acid-BBN(7-14) wherein the BBN(7-14) sequence is SEQ ID NO: 1;
-monoamide-Gly-3-methoxy-4-aminobenzoic acid-BBN(7-14) wherein the BBN(7-14) sequence is SEQ ID NO: 1;
-monoamide-Gly-3-chloro-4-aminobenzoic acid-BBN(7-14) wherein the BBN(7-14) sequence is SEQ ID NO: 1;
-monoamide-Gly-3-methyl-4-aminobenzoic acid-BBN(7-14) wherein the BBN(7-14) sequence is SEQ ID NO: 1;
-monoamide-Gly-3-hydroxy-4-aminobenzoic acid-BBN(7-14) wherein the BBN(7-14) sequence is SEQ ID NO: 1;
A-monoamide)₂-N,N′-Bis(2-aminoethyl)-succinamic acid-BBN(7-14) wherein the BBN(7-14) sequence is SEQ ID NO: 1;
-monoamide-G-4-aminobenzoic acid-QWAVGHFL-NH₂ wherein QWAVGHFL-NH₂ is SEQ ID NO: 22;
-monoamide-4-aminomethylbenzoic acid-L-1-Naphthylalanine-QWAVGHLM-NH₂ wherein QWAVGHLM-NH₂ is SEQ ID NO: 1;
-monoamide-G-4-aminobenzoic acid-QWAVGNMeHisLM-NH₂ wherein QWAVGNMeHisLM-NH₂ is SEQ ID NO: 15;
-Gly-4-aminobenzoic acid-BBN(7-14) wherein the BBN(7-14) sequence is SEQ ID NO: 1; and
zta-Gly-4-aminobenzoic acid-BBN(7-14) wherein the BBN(7-14) sequence is SEQ ID NO: 1.

154. The method of embodiment 146, wherein the labeled compound comprise DO3A-monoamide-Gly-4-aminobenzoic acid-BBN(7-14) wherein the BBN(7-14) sequence is SEQ ID NO: 1.

155. A method for increasing the targeting of a labeled compound to a GRP receptor expressing target tissue within a subject comprising the step of administering to a subject a dose comprising a combination of:

a first GRP receptor targeting peptide to occupy a GRP receptor binding site in non-target tissue, and

a labeled compound of the general formula:

M-N—O—P-G

wherein

M is an optical label or a metal chelator complexed with a radionuclide;

N is 0, an alpha or non-alpha amino acid or other linking group;

O is an alpha or non-alpha amino acid; and

P is 0, an alpha or non-alpha amino acid or other linking group,

and G is a second GRP receptor targeting peptide, and

wherein at least one of N, O or P is a non-alpha amino acid.

156. The method of embodiment 155, wherein the first GRP receptor targeting peptide is conjugated to a linker.

157. The method of embodiment 155, wherein the first GRP receptor targeting peptide is conjugated to a linker which is attached to one or more metal chelator.

158. The method of embodiment 157, wherein said first GRP receptor peptide, linker and metal chelator are the same as the second GRP receptor targeting peptide, N—O—P linker and metal chelator of the labeled compound.

159. The method of embodiment 158, wherein the non-alpha amino acid is selected from the group consisting of:
8-amino-3,6-dioxaoctanoic acid;
N-4-aminoethyl-N-1-piperazine-acetic acid; and
   polyethylene glycol derivatives having the formula NH₂—(CH₂CH₂O)n-CH₂CO₂H or NH₂—(CH₂CH₂O)n-CH₂CH₂CO₂H where n=2 to 100.

160. The method of embodiment 155, wherein the metal chelator is selected from the group consisting of DTPA, DOTA, DO3A, HP-DO3A, EDTA, TETA, EHPG, HBED, NOTA, DOTMA, TETMA, PDTA, TTHA, LICAM, MECAM, CMDOTA, N,N-dimethylGly-Ser-Cys, Aazta and derivatives thereof.

161. The method of embodiment 155, wherein the labeled compound comprises a compound selected from the group consisting of:

methylglycine-Ser-Cys(Acm)-Gly-Lys-8-amino-3,6-dioxaoctanoic acid-BBN(7-14) wherein the BBN(7-14) sequence is SEQ ID NO: 1;
methylglycine-Ser-Cys(Acm)-Gly-Arg-8-amino-3,6-dioxaoctanoic acid-BBN(7-14) wherein the BBN(7-14) sequence is SEQ ID NO: 1;
methylglycine-Ser-Cys(Acm)-Gly-Asp-8-amino-3,6-dioxaoctanoic acid-BBN(7-14) wherein the BBN(7-14) sequence is SEQ ID NO: 1;
methylglycine-Ser-Cys(Acm)-Gly-Ser-8-amino-3,6-dioxaoctanoic acid-BBN(7-14) wherein the BBN(7-14) sequence is SEQ ID NO: 1;
methylglycine-Ser-Cys(Acm)-Gly-Gly-8-amino-3,6-dioxaoctanoic acid-BBN(7-14) wherein the BBN(7-14) sequence is SEQ ID NO: 1;
methylglycine-Ser-Cys(Acm)-Gly-Glu-8-amino-3,6-dioxaoctanoic acid-BBN(7-14) wherein the BBN(7-14) sequence is SEQ ID NO: 1;
methylglycine-Ser-Cys(Acm)-Gly-Dala-8-amino-3,6-dioxaoctanoic acid-BBN(7-14) wherein the BBN(7-14) sequence is SEQ ID NO: 1;
methylglycine-Ser-Cys(Acm)-Gly-8-amino-3,6-dioxaoctanoic acid-Lys-BBN(7-14) wherein the BBN(7-14) sequence is SEQ ID NO: 1;
methylglycine-Ser-Cys(Acm)-Gly-8-amino-3,6-dioxaoctanoic acid-Arg-BBN(7-14) wherein the BBN(7-14) sequence is SEQ ID NO: 1;
methylglycine-Ser-Cys(Acm)-Gly-8-amino-3,6-dioxaoctanoic acid-Asp-BBN(7-14) wherein the BBN(7-14) sequence is SEQ ID NO: 1;
methylglycine-Ser-Cys(Acm)-Gly-8-amino-3,6-dioxaoctanoic acid-Ser-BBN(7-14) wherein the BBN(7-14) sequence is SEQ ID NO: 1;
methylglycine-Ser-Cys(Acm)-Gly-8-amino-3,6-dioxaoctanoic acid-Gly-BBN(7-14) wherein the BBN(7-14) sequence is SEQ ID NO: 1;
nethylglycine-Ser-Cys(Acm)-Gly-8-amino-3,6-dioxaoctanoic acid-Glu-BBN(7-14) wherein the BBN(7-14) sequence is SEQ ID NO: 1;
methylglycine-Ser-Cys(Acm)-Gly-8-amino-3,6-dioxaoctanoic acid-Dala-BBN(7-14) wherein the BBN(7-14) sequence is SEQ ID NO: 1;
methylglycine-Ser-Cys(Acm)-Gly-8-amino-3,6-dioxaoctanoic acid-8-amino-3,6-dioxaoctanoic acid-BBN(7-14) wherein the BBN(7-14) sequence is SEQ ID NO: 1;
methylglycine-Ser-Cys(Acm)-Gly-8-amino-3,6-dioxaoctanoic acid-2,3-diaminopropionic acid-BBN(7-14) wherein the BBN(7-14) sequence is SEQ ID NO: 1;
methylglycine-Ser-Cys(Acm)-Gly-8-amino-3,6-dioxaoctanoic acid-BBN(7-14) wherein the BBN(7-14) sequence is SEQ ID NO: 1;
methylglycine-Ser-Cys(Acm)-Gly-2,3-diaminopropionic acid-8-amino-3,6-dioxaoctanoic acid-BBN(7-14) wherein the BBN(7-14) sequence is SEQ ID NO: 1;
methylglycine-Ser-Cys(Acm)-Gly-Asp-8-amino-3,6-dioxaoctanoic acid-Gly-BBN(7-14) wherein the BBN(7-14) sequence is SEQ ID NO: 1;
methylglycine-Ser-Cys(Acm)-Gly-8-amino-3,6-dioxaoctanoic acid-Asp-Gly-BBN(7-14) wherein the BBN(7-14) sequence is SEQ ID NO: 1;
methylglycine-Ser-Cys(Acm)-Gly-8-amino-3,6-dioxaoctanoic acid-Ser-Gly-BBN(7-14) wherein the BBN(7-14) sequence is SEQ ID NO: 1;
methylglycine-Ser-Cys(Acm)-Gly-8-amino-3,6-dioxaoctanoic acid-Arg-Gly-BBN(7-14) wherein the BBN(7-14) sequence is SEQ ID NO: 1;
methylglycine-Ser-Cys(Acm)-Gly-8-amino-3,6-dioxaoctanoic acid-8-amino-3,6-dioxaoctanoic acid-Gly-BBN(7-14) wherein the BBN(7-14) sequence is SEQ ID NO: 1;
methylglycine-Ser-Cys(Acm)-Gly-8-amino-3,6-dioxaoctanoic acid-2,3-diaminopropionic acid-Gly-BBN(7-14) wherein the BBN(7-14) sequence is SEQ ID NO: 1;
methylglycine-Ser-Cys(Acm)-Gly-8-amino-3,6-dioxaoctanoic acid-Lys-Gly-BBN(7-14) wherein the BBN(7-14) sequence is SEQ ID NO: 1;
methylglycine-Ser-Cys(Acm)-Gly-2,3-diaminopropionic acid-8-amino-3,6-dioxaoctanoic acid-Gly-BBN(7-14) wherein the BBN(7-14) sequence is SEQ ID NO: 1;
methylglycine-Ser-Cys(Acm)-Gly-N-4-aminoethyl-N-1-piperazineacetic acid-Asp-BBN(7-14) wherein the BBN(7-14) sequence is SEQ ID NO: 1;
methylglycine-Ser-Cys(Acm)-Gly-N-4-aminoethyl-N-1-piperazineacetic acid-Ser-BBN(7-14) wherein the BBN(7-14) sequence is SEQ ID NO: 1;
methylglycine-Ser-Cys(Acm)-Gly-N-4-aminoethyl-N-1-piperazineacetic acid-Arg-BBN(7-14) wherein the BBN(7-14) sequence is SEQ ID NO: 1;
methylglycine-Ser-Cys(Acm)-Gly-N-4-aminoethyl-N-1-piperazineacetic acid-8-amino-3,6-dioxaoctanoic acid-BBN(7-14) wherein the BBN(7-14) sequence is SEQ ID NO: 1;
methylglycine-Ser-Cys(Acm)-Gly-N-4-aminoethyl-N-1-piperazineacetic acid-2,3-diaminopropionic acid-BBN(7-14) wherein the BBN(7-14) sequence is SEQ ID NO: 1;
methylglycine-Ser-Cys(Acm)-Gly-N-4-aminoethyl-N-1-piperazineacetic acid-Lys-BBN(7-14) wherein the BBN(7-14) sequence is SEQ ID NO: 1;
methylglycine-Ser-Cys(Acm)-Gly-N-1-piperazineacetic acid-Asp-BBN(7-14) wherein the BBN(7-14) sequence is SEQ ID NO: 1;
methylglycine-Ser-Cys(Acm)-Gly-N-1-piperazineacetic acid-Ser-BBN(7-14) wherein the BBN(7-14) sequence is SEQ ID NO: 1;
methylglycine-Ser-Cys(Acm)-Gly-N-1-piperazineacetic acid-Arg-BBN(7-14) wherein the BBN(7-14) sequence is SEQ ID NO: 1;
methylglycine-Ser-Cys(Acm)-Gly-N-1-piperazineacetic acid-8-amino-3,6-dioxaoctanoic acid-BBN(7-14) wherein the BBN(7-14) sequence is SEQ ID NO: 1;
methylglycine-Ser-Cys(Acm)-Gly-N-1-piperazineacetic acid-2,3-diaminopropionic acid BBN(7-14) wherein the BBN(7-14) sequence is SEQ ID NO: 1;
methylglycine-Ser-Cys(Acm)-Gly-N-1-piperazineacetic acid-Lys-BBN(7-14) wherein the BBN(7-14) sequence is SEQ ID NO: 1;
methylglycine-Ser-Cys(Acm)-Gly-4-Hydroxyproline-8-amino-3,6-dioxaoctanoic acid-BBN(7-14) wherein the BBN(7-14) sequence is SEQ ID NO: 1;
methylglycine-Ser-Cys(Acm)-Gly-4-aminoproline-8-amino-3,6-dioxaoctanoic acid-BBN(7-14) wherein the BBN(7-14) sequence is SEQ ID NO: 1;
methylglycine-Ser-Cys(Acm)-Gly-Lys-8-amino-3,6-dioxaoctanoic acid-Gly-BBN(7-14) wherein the BBN(7-14) sequence is SEQ ID NO: 1;
methylglycine-Ser-Cys(Acm)-Gly-Arg-8-amino-3,6-dioxaoctanoic acid-Gly-BBN(7-14) wherein the BBN(7-14) sequence is SEQ ID NO: 1;
methylglycine-Ser-Cys(Acm)-Gly-Ser-8-amino-3,6-dioxaoctanoic acid-Gly-BBN(7-14) wherein the BBN(7-14) sequence is SEQ ID NO: 1;
methylglycine-Ser-Cys(Acm)-Gly-Asp-8-amino-3,6-dioxaoctanoic acid-Gly-BBN(7-14) wherein the BBN(7-14) sequence is SEQ ID NO: 1;
methylglycine-Ser-Cys(Acm)-Gly-8-amino-3,6-dioxaoctanoic acid-Asp-Gly-BBN(7-14) wherein the BBN(7-14) sequence is SEQ ID NO: 1;
methylglycine-Ser-Cys(Acm)-Gly-8-amino-3,6-dioxaoctanoic acid-Ser-Gly-BBN(7-14) wherein the BBN(7-14) sequence is SEQ ID NO: 1;
methylglycine-Ser-Cys(Acm)-Gly-8-amino-3,6-dioxaoctanoic acid-Arg-Gly-BBN(7-14) wherein the BBN(7-14) sequence is SEQ ID NO: 1;
methylglycine-Ser-Cys(Acm)-Gly-8-amino-3,6-dioxaoctanoic acid-8-amino-3,6-dioxaoctanoic acid-Gly-BBN(7-14) wherein the BBN(7-14) sequence is SEQ ID NO: 1;
methylglycine-Ser-Cys(Acm)-Gly-8-amino-3,6-dioxaoctanoic acid-2,3-diaminopropionic acid-Gly-BBN(7-14) wherein the BBN(7-14) sequence is SEQ ID NO: 1;
methylglycine-Ser-Cys(Acm)-Gly-8-amino-3,6-dioxaoctanoic acid-Lys-Gly-BBN(7-14) wherein the BBN(7-14) sequence is SEQ ID NO: 1; and
methylglycine-Ser-Cys(Acm)-Gly-2,3-diaminopropionic acid-8-amino-3,6-dioxaoctanoic acid-Gly-BBN(7-14) wherein the BBN(7-14) sequence is SEQ ID NO: 1.

162. The method of embodiment 155, wherein the labeled compound comprises a compound selected from the group consisting of:

-monoamide-8-amino-3,6-dioxaoctanoic acid-diaminopropionic acid-BBN(7-14) wherein the BBN(7-14) sequence is SEQ ID NO: 1;
-monoamide-8-amino-3,6-dioxaoctanoic acid-biphenylalanine-BBN(7-14) wherein the BBN(7-14) sequence is SEQ ID NO: 1;
-monoamide-8-amino-3,6-dioxaoctanoic acid-diphenylalanine-BBN(7-14) wherein the BBN(7-14) sequence is SEQ ID NO: 1;
-monoamide-8-amino-3,6-dioxaoctanoic acid-4-benzoylphenylalanine-BBN(7-14) wherein the BBN(7-14) sequence is SEQ ID NO: 1;
-monoamide-5-aminopentanoic acid-8-amino-3,6-dioxaoctanoic acid-BBN(7-14) wherein the BBN(7-14) sequence is SEQ ID NO: 1;
-monoamide-8-amino-3,6-dioxaoctanoic acid-D-phenylalanine-BBN(7-14) wherein the BBN(7-14) sequence is SEQ ID NO: 1;
-monoamide-8-aminooctanoic acid-8-amino-3,6-dioxaoctanoic acid-BBN(7-14) wherein the BBN(7-14) sequence is SEQ ID NO: 1;
-monoamide-E(G8-amino-3,6-dioxaoctanoic acid-8-amino-3,6-dioxaoctanoic acid QWAVGHLM-NH₂)-8-aminooctanoic acid-8-aminooctanoic acid-BBN(7-14) wherein the BBN(7-14) sequence is SEQ ID NO: 1; and
-monoamide-E(G-Aoa-Aoa-QWAVGHLM-NH₂)-8-aminooctanoic acid-8-aminooctanoic acid-BBN(7-14) wherein the BBN(7-14) sequence is SEQ ID NO: 1.

163. A method for increasing the targeting of a labeled compound to a GRP receptor expressing target tissue within a subject comprising the step of administering to a subject a dose comprising a combination of:

a first GRP receptor targeting peptide to occupy a GRP receptor binding site in non-target tissue, and

a labeled compound of the general formula:

M-N—O—P-G
wherein
   M is an optical label or a metal chelator complexed with a radionuclide;
   N is 0, an alpha amino acid, a substituted bile acid or other linking group;
   O is an alpha amino acid or a substituted bile acid;
   P is 0, an alpha amino acid, a substituted bile acid or other linking group;

G is a second GRP receptor targeting peptide, and

wherein at least one of N, O or P is a substituted bile acid.

164. The method of embodiment 163, wherein the first GRP receptor targeting peptide is conjugated to a linker.

165. The method of embodiment 163, wherein the first GRP receptor targeting peptide is conjugated to a linker which is attached to one or more metal chelator.

166. The method of embodiment 166, wherein said first GRP receptor peptide, linker and metal chelator are the same as the second GRP receptor targeting peptide, N—O—P linker and metal chelator of the labeled compound.

167. The method of embodiment 163, wherein the substituted bile acid is selected from the group consisting of:
3β-amino-3-deoxycholic acid;
(3β,5β)-3-aminocholan-24-oic acid;
(3β,5β,12α)-3-amino-12-hydroxycholan-24-oic acid;
(3β,5β,7α,12α)-3-amino-7,12-dihydroxycholan-24-oic acid;
Lys-(3,6,9)-trioxaundecane-1,1′-dicarbonyl-3,7-dideoxy-3-aminocholic acid);
(3β,5β,7α,12α)-3-amino-7-hydroxy-12-oxocholan-24-oic acid; and
(3β,5β,7α)-3-amino-7-hydroxycholan-24-oic acid.

168. The method of embodiment 163, wherein the metal chelator is selected from the group consisting of: DTPA, DOTA, DO3A, HPDO3A, EDTA, TETA, CMDOTA, N,N-dimethylGly-Ser-Cys, Aazta and derivatives thereof.

169. The method of embodiment 163, wherein the labeled compound comprises a compound selected from the group consisting of:

[symbol]

-monoamide-Gly-(3β,5β)-3-aminocholan-24-oic acid-BBN(7-14) wherein the BBN(7-14) sequence is SEQ ID NO: 1;
-monoamide-Gly-(3β,5β,12α)-3-amino-12-hydroxycholan-24-oic acid-BBN(7-14) wherein the BBN(7-14) sequence is SEQ ID NO: 1;
-monoamide-Gly-(3β,5β,7α,12α)-3-amino-7,12-dihydroxycholan-24-oic acid-BBN(7-14) wherein the BBN(7-14) sequence is SEQ ID NO: 1;
-monoamide-Gly-Lys-(3,6,9)-trioxaundecane-1,1′-dicarbonyl-3,7-dideoxy-3-aminocholic acid)-Arg-BBN(7-14) wherein the BBN(7-14) sequence is SEQ ID NO: 1;
,7α,12α)-3-amino-7,12-dihydroxycholan-24-oic acid-3,6,9-trioxaundecane-1,1′-dicarbonyl Lys(DO3A-monoamide-Gly)-Arg-BBN(7-14) wherein the BBN(7-14) sequence is SEQ ID NO: 1;
   -monoamide-Gly-(3β,5β,7α,12α)-3-amino-12-oxocholan-24-oic acid-BBN(7-14) wherein the BBN(7-14) sequence is SEQ ID NO: 1;
   -monoamide-1-amino-3,6-dioxaoctanoic acid-(3β,5β,7α,12α)-3-amino-7,12-dihydroxycholan-24-oic acid-BBN(7-14) wherein the BBN(7-14) sequence is SEQ ID NO: 1;
   -monoamide-Gly-(3β,5β,7α,12α)-3-amino-7,12-dihydroxycholan-24-oic acid-QWAVaHLM-NH₂ wherein QWAVaHLM-NH₂ is SEQ ID NO: 14;
   -monoamide-Gly-(3β,5β,7α,12α)-3-amino-7,12-dihydroxycholan-24-oic acid-f-QWAVGHLM-NH: wherein QWAVGHLM-NH₂ is SEQ ID NO: 1;
   -monoamide-Gly-(3β,5β,7α,12α)-3-amino-7,12-dihydroxycholan-24-oic acid-f-WAVGHLL-NH₂ wherein WAVGHLL-NH₂ is SEQ ID NO: 26;
   -monoamide-Gly-(3β,5β,7α,12α)-3-amino-7,12-dihydroxycholan-24-oic acid-f-QWAVGHL-NH-pentyl wherein QWAVGHL-NH-pentyl is SEQ ID NO: 6;
   -monoamide-Gly-(3β,5β,7α,12α)-3-amino-7,12-dihydroxycholan-24-oic acid-γ-QWAV-Bala-H-F-Nle-NH₂ wherein QWAV-Bala-H-F-Nle-NH₂ is SEQ ID NO: 9;
   -monoamide-Gly-(3β,5β,7α,12α)-3-amino-7,12-dihydroxycholan-24-oic acid-f-QWAV-Bala-H-F-Nle-NH₂ wherein QWAV-Bala-H-F-Nle-NH₂ is SEQ ID NO: 9;
   -monoamide-Gly-(3β,5β,7α,12α)-3-amino-7,12-dihydroxycholan-24-oic acid-QWAVGHFL-NH₂ wherein QWAVGHFL-NH₂ is SEQ ID NO: 22;
   -monoamide-Gly-(3β,5β,7α,12α)-3-amino-7,12-dihydroxycholan-24-oic acid-QWAVGNMeH-L-M-NH₂ wherein QWAVGNMeH-L-M-NH₂ wherein QWAVGNMeH-L-M-NH₂ is SEQ ID NO: 15
   -monoamide-Gly-(3β,5β,7α,12α)-3-amino-7,12-dihydroxycholan-24-oic acid-LWAVGSF-M-NH₂ wherein LWAVGSF-M-NH₂ is SEQ ID NO: 11;
   -monoamide-Gly-(3β,5β,7α,12α)-3-amino-7,12-dihydroxycholan-24-oic acid-HWAVGHLM-NH₂ wherein HWAVGHLM-NH₂ is SEQ ID NO: 12;
   -monoamide-Gly-(3β,5β,7α,12α)-3-amino-7,12-dihydroxycholan-24-oic acid-LWAGHFM-NH₂ wherein LWAGHFM-NH₂ is SEQ ID NO: 20;
   -monoamide-Gly-(3β,5β,7α,12α)-3-amino-7,12-dihydroxycholan-24-oic acid-QWAVGHFM-NH₂ wherein QWAVGHFM-NH₂ is SEQ ID NO: 13;
   -monoamide-Gly-(3β,5β,7α,12α)-3-amino-7,12-dihydroxycholan-24-oic acid-QRLGNQWAVGHLM-NH₂ wherein QRLGNQWAVGHLM-NH₂ is SEQ ID NO: 3;
   -monoamide-Gly-(3β,5β,7α,12α)-3-amino-7,12-dihydroxycholan-24-oic acid-QRYGNQWAVGHLM-NH₂ wherein QRYGNQWAVGHLM-NH₂ is SEQ ID NO: 4;
   -monoamide-Gly-(3β,5β,7α,12α)-3-amino-7,12-dihydroxycholan-24-oic acid-QKYGNQWAVGHLM-NH₂ wherein QKYGNQWAVGHLM-NH₂ is SEQ ID NO: 5;
-Lys (DO3A-monoamide)-Gly-(3β,5β,7α,12α)-3-amino-7,12-dihydroxycholan-24-oic acid-LGNQWAVGHLM-NH₂ wherein LGNQWAVGHLM-NH₂ is SEQ ID NO: 18;
   -monoamide-Gly-3-amino-3-deoxycholic acid-QRLGNQWAVGHLM-NH₂ wherein QRLGNQWAVGHLM-NH₂ is SEQ ID NO: 3;
   -monoamide-Gly-3-amino-3-deoxycholic acid-QRYGNQWAVGHLM-NH₂ wherein QRYGNQWAVGHLM-NH₂ is SEQ ID NO: 4;
   -monoamide-Gly-3-amino-3-deoxycholic acid-QKYGNQWAVGHLM-NH₂ wherein QKYGNQWAVGHLM-NH₂ is SEQ ID NO: 5;
   -Lys(DO3A-monoamide-G-3-amino-3-deoxycholic acid)-LGNQWAVGHLM-NH₂ wherein LGNQWAVGHLM-NH₂ is SEQ ID NO: 18;
   Gly-(3β,5α,7α,12α)-3-amino-7,12-dihydroxycholan-24-oic acid-BBN(7-14) wherein the BBN(7-14 sequence is SEQ ID NO: 1; and
   ta-Gly-(3β,5β,7α,12α)-3-amino-7,12-dihydroxycholan-24-oic acid-BBN(7-14) wherein the BBN(7-14) sequence is SEQ ID NO: 1.

170. The method of embodiment 163, wherein said labeled compound comprises DO3A-monoamide-Gly-(3β,5β,7α,12α)-3-amino-7,12-dihydroxycholan-24-oic acid-BBN(7-14) wherein the BBN(7-14) sequence is SEQ ID NO: 1.

171. A method for increasing the targeting of a labeled compound to a GRP receptor expressing target tissue within a subject comprising the step of administering to a subject a dose comprising a combination of:

a first GRP receptor targeting peptide to occupy a GRP receptor binding site in non-target tissue, and
a labeled compound of the general formula:

M-N—O—P-G

wherein
M is an optical label or a metal chelator complexed with a radionuclide;
N is 0, an alpha amino acid, a non-alpha amino acid with a cyclic group or other linking group;
O is an alpha amino acid or a non-alpha amino acid with a cyclic group;
P is 0, an alpha amino acid, a non-alpha amino acid with a cyclic group, or other linking group; and
G is a second GRP receptor targeting peptide,
wherein at least one of N, O or P is a non-alpha amino acid with a cyclic group.

172. The method of embodiment 171, wherein the first GRP receptor targeting peptide is conjugated to a linker.

173. The method of embodiment 171,wherein the first GRP receptor targeting peptide is conjugated to a linker which is attached to one or more metal chelator.

174. The method of embodiment 173, wherein said first GRP receptor peptide, linker and metal chelator are the same as the GRP receptor targeting peptide, N—O—P linker and metal chelator of the labeled compound.

175. The method of embodiment 171, wherein the non-alpha amino acid with a cyclic group is selected from the group consisting of:
4-aminobenzoic acid;
4-aminomethyl benzoic acid;
trans-4-aminomethylcyclohexane carboxylic acid;
4-(2-aminoethoxy)benzoic acid;
Isonipecotic acid;
2-aminomethylbenzoic acid;
4-amino-3-nitrobenzoic acid;
4-(3-carboxymethyl-2-keto-1-benzimidazolyl)-piperidine;
6-(piperazin-1-yl)-4-(3H)-quinazolinone-3-acetic acid;
-5-amino-1,2,4,5,6,7-hexahydro-azepino[3,2,1-hi]indole-4-one-2-carboxylic acid;
[symbol] ,)-4-amino-6-aza-5-oxo-9-thiabicyclo[4.3.0]nonane-7-carboxylic acid;
3-carboxymethyl-1-phenyl-1,3,8-triazaspiro[4.5]decan-4-one;
N1-piperazineacetic acid;
N-4-aminoethyl-N-1-acetic acid;
(3S)-3-amino-1-carboxymethylcaprolactam; and
,9)-6-amino-2-carboxymethyl-3,8-diazabicyclo-[4,3,O]-nonane-1,4-dione.

176. The method of embodiment 171, wherein the metal chelator is selected from the group consisting of: DTPA, DOTA, DO3A, HPDO3A, EDTA, TETA, N,N-dimethylGly-Ser-Cys, Aazta and derivatives thereof.

177. The method of embodiment 171, wherein the labeled compound comprises a compound selected from the group consisting of

-monoamide-Gly-4-aminobenzoic acid-BBN(7-14) wherein the BBN(7-14) sequence is SEQ ID NO: 1;
-monoamide-4-aminomethyl benzoic acid-BBN(7-14) wherein the BBN(7-14) sequence is SEQ ID NO: 1;
-monoamide-trans-4-aminomethylcyclohexyl carboxylic acid-BBN(7-14) wherein the BBN(7-14) sequence is SEQ ID NO: 1;
-monoamide-4-(2-aminoethoxy)benzoic acid-BBN(7-14) wherein the BBN(7-14) sequence is SEQ ID NO: 1;
-monoamide-Gly-isonipecotic acid-BBN(7-14) wherein the BBN(7-14) sequence is SEQ ID NO: 1;
-monoamide-2-aminomethylbenzoic acid-BBN(7-14) wherein the BBN(7-14) sequence is SEQ ID NO: 1;
-monoamide-4-aminomethyl-3-nitrobenzoic acid-BBN(7-14) wherein the BBN(7-14) sequence is SEQ ID NO: 1;
-monoamide-8-amino-3,6-dioxaoctanoic acid-1-naphthylalanine-BBN(7-14) wherein the BBN(7-14) sequence is SEQ ID NO: 1;
-monoamide-4-(3-carboxymethyl-2-keto-1-benzimidazolyl-piperidine-BBN(7-14) wherein the BBN(7-14) sequence is SEQ ID NO: 1;
-monoamide-6-(piperazin-1-yl)-4-(3H)-quinazolinone-3-acetic acid-BBN(7-14) wherein the BBN(7-14) sequence is SEQ ID NO: 1;
-monoamide-(2S,5S)-5-amino-1,2,4,5,6,7-hexahydro-azepino[3,2,1-hi]indole-4-one-2-carboxylic acid-BBN(7-14) wherein the BBN(7-14) sequence is SEQ ID NO: 1;
-monoamide-(4S,7R)-4-amino-6-aza-5-oxo-9-thiabicyclo[4.3.0]nonane-7-carboxylic acid-BBN(7-14) wherein the BBN(7-14) sequence is SEQ ID NO: 1;
-monoamide-N,N-dimethylglycine-BBN(7-14) wherein the BBN(7-14) sequence is SEQ ID NO: 1;
-monoamide-3-carboxymethyl-1-phenyl-1,3,8-triazaspiro[4.5]decan-4-one-BBN(7-14) wherein the BBN(7-14) sequence is SEQ ID NO: 1;
-monoamide-N1-piperazineacetic acid-BBN(7-14) wherein the BBN(7-14) sequence is SEQ ID NO: 1;
-monoamide-N-4-aminoethyl-N-1-piperazineacetic acid-BBN(7-14) wherein the BBN(7-14) sequence is SEQ ID NO: 1;
-monoamide-(3S)-3-amino-1-carboxymethylcaprolactam-BBN(7-14) wherein the BBN(7-14) sequence is SEQ ID NO: 1;
-monoamide-(2S,6S,9)-6-amino-2-carboxymethyl-3,8-diazabicyclo-[4,3,0]-nonane-1,4-dione-BBN(7-14) wherein the BBN(7-14) sequence is SEQ ID NO: 1;
-monoamide-5-aminopentanoic acid-trans-4-aminomethylcyclohexane-1-carboxylic acid-BBN(7-14) wherein the BBN(7-14) sequence is SEQ ID NO: 1;
-monoamide-trans-4-aminomethylcyclohexane-1-carboxylic acid-D-phenylalanine-BBN(7-14) wherein the BBN(7-14) sequence is SEQ ID NO: 1;
-monoamide-4-aminomethylbenzoic acid-8-amino-3,6-dioxaoctanoic acid-BBN(7-14) wherein the BBN(7-14) sequence is SEQ ID NO: 1;
-monoamide-4-benzoyl-(L)-phenylalanine-trans-4-aminomethylcyclohexane-1-carboxylic acid-BBN(7-14) wherein the BBN(7-14) sequence is SEQ ID NO: 1;
-monoamide-trans-4-aminomethylcyclohexane-1-carboxylic acid-Arg-BBN(7-14) wherein the BBN(7-14) sequence is SEQ ID NO: 1;
-monoamide-trans-4-aminomethylcyclohexane-1-carboxylic acid-Lys-BBN(7-14) wherein the BBN(7-14) sequence is SEQ ID NO: 1;
-monoamide-trans-4-aminomethylcyclohexane-1-carboxylic acid-diphenylalanine-BBN(7-14) wherein the BBN(7-14) sequence is SEQ ID NO: 1;
-monoamide-trans-4-aminomethylcyclohexane-1-carboxylic acid-1-naphthylalanine-BBN(7-14) wherein the BBN(7-14) sequence is SEQ ID NO: 1;
-monoamide-trans-4-aminomethylcyclohexane-1-carboxylic acid-8-amino-3,6-dioxaoctanoic acid-BBN(7-14) wherein the BBN(7-14) sequence is SEQ ID NO: 1;
-monoamide-trans-4-aminomethylcyclohexane-1-carboxylic acid-Ser-BBN(7-14) wherein the BBN(7-14) sequence is SEQ ID NO: 1;
-monoamide-trans-4-aminomethylcyclohexane-1-carboxylic acid-2,3-diaminopropionic acid-BBN(7-14) wherein the BBN(7-14) sequence is SEQ ID NO: 1;
-monoamide-trans-4-aminomethylcyclohexane-1-carboxylic acid-biphenylalanine-BBN(7-14) wherein the BBN(7-14) sequence is SEQ ID NO: 1;
-monoamide-trans-4-aminomethylcyclohexane-1-carboxylic acid-(2S,5S)-5-amino-1,2,4,5,6,7-hexahydro-azepino[3,21-hi]indole-4-one-2-carboxylic acid-BBN(7-14) wherein the BBN(7-14) sequence is SEQ ID NO: 1;
-monoamide-trans-4-aminomethylcyclohexane-1-carboxylic acid-trans-4-aminomethylcyclohexane-1-carboxylic acid-BBN(7-14) wherein the BBN(7-14) sequence is SEQ ID NO: 1;
-monoamide-8-amino-3,6-dioxaoctanoic acid-phenylalanine-BBN(7-14) wherein the BBN(7-14) sequence is SEQ ID NO: 1;
-monoamide-trans-4-aminomethylcyclohexane-1-carboxylic acid-phenylalanine-BBN(7-14) wherein the BBN(7-14) sequence is SEQ ID NO: 1;
-monoamide-8-aminooctanoic acid-trans-4-aminomethylcyclohexane-1-carboxylic acid-BBN(7-14) wherein the BBN(7-14) sequence is SEQ ID NO: 1;
-monoamide-4′-aminomethyl-biphenyl-1-carboxylic acid-BBN(7-14) wherein the BBN(7-14) sequence is SEQ ID NO: 1;
-monoamide-3′-aminomethyl-biphenyl-3-carboxylic acid-BBN(7-14) wherein the BBN(7-14) sequence is SEQ ID NO: 1;
)TA-Gly-4-aminobenzoic acid-BBN(7-14) wherein the BBN(7-14) sequence is SEQ ID NO: 1;
-monoamide-4-aminomethylphenoxyacetic acid-BBN(7-14) wherein the BBN(7-14) sequence is SEQ ID NO: 1;
-monoamide-Gly-4-aminophenylacetic acid-BBN(7-14) wherein the BBN(7-14) sequence is SEQ ID NO: 1;
3A-4-phenoxy-BBN(7-14) wherein the BBN(7-14) sequence is SEQ ID NO: 1;
-monoamide-3-aminomethylbenzoic acid-BBN(7-14) wherein the BBN(7-14) sequence is SEQ ID NO: 1;
-monoamide-4-aminomethylphenylacetic acid-BBN(7-14) wherein the BBN(7-14) sequence is SEQ ID NO: 1;
-monoamide-4-aminomethyl-3-methoxybenzoic acid-BBN(7-14) wherein the BBN(7-14) sequence is SEQ ID NO: 1;
ly-4-aminobenzoic acid-BBN(7-14) wherein the BBN(7-14) sequence is SEQ ID NO: 1;
-monoamide-Gly-4-hydrazinobenzoyl-BBN(7-14) wherein the BBN(7-14) sequence is SEQ ID NO: 1;
-monoamide-4-aminobenzoic acid-BBN(7-14) wherein the BBN(7-14) sequence is SEQ ID NO: 1;
-monoamide-4-aminobenzoic acid-Gly-BBN(7-14) wherein the BBN(7-14) sequence is SEQ ID NO: 1;
-monoamide-Gly-6-Aminonicotinic acid-BBN(7-14) wherein the BBN(7-14) sequence is SEQ ID NO: 1;
-monoamide-Gly-4′-Amino-2′-methyl biphenyl-4-carboxylic acid-BBN(7-14) wherein the BBN(7-14) sequence is SEQ ID NO: 1;
-monoamide-Gly-3′-Aminobiphenyl-3-carboxylic acid-BBN(7-14) wherein the BBN(7-14) sequence is SEQ ID NO: 1;
-monoamide-Gly-1,2-diaminoethyl-Terephthalic acid-BBN(7-14) wherein the BBN(7-14) sequence is SEQ ID NO: 1;
-monoamide-Gly-Gly-4-aminobenzoic acid-BBN(7-14) wherein the BBN(7-14) sequence is SEQ ID NO: 1;
-monoamide-G-4-aminobenzoic acid-EWAVGHLM-NH₂ wherein EWAVGHLM-NH₂ is SEQ ID NO: 2;
-monoamide-G-4-aminobenzoic acid-QWAVGHLM-OH wherein QWAVGHLM-OH is SEQ ID NO: 1;
-monoamide-G-4-aminobenzoic acid-(D)-Phe-BBN(7-14) wherein the BBN(7-14) sequence is SEQ ID NO: 1;
-monoamide-G-4-aminobenzoic acid-QRLGNQWAVGHLM-NH₂ wherein QRLGNQWAVGHLM-NH₂ is SEQ ID NO: 3;
-monoamide-G-4-aminobenzoic acid-QRYGNQWAVGHLM-NH₂ wherein QRYGNQWAVGHLM-NH₂ is SEQ ID NO: 4;
-monoamide-G-4-aminobenzoic acid-QKYGNQWAVGHLM-NH₂ wherein QKYGNQWAVGHLM-NH₂ is SEQ ID NO: 5;
-monoamide-G-4-aminobenzoic acid-(D)-Phe-QWAVGHL-NH-Pentyl wherein QWAVGHL-NH-Pentyl is SEQ ID NO: 6;
-monoamide-G-4-aminobenzoic acid-QWSVaHLM-NH₂ wherein QWSVaHLM-NH₂ is SEQ ID NO: 7;
-monoamide-G-4-aminobenzoic acid-(D)-Phe-QWAVGHLL-NH₂ wherein QWAVGHLL-NH₂ is SEQ ID NO: 8;
-monoamide-G-4-aminobenzoic acid-(D)-Tyr-QWAV-Bala-HF-Nle-NH₂ wherein QWAV-Bala-HF-Nle-NH₂ is SEQ ID NO: 9;
-monoamide-G-4-aminobenzoic acid-Phe-QWAV-Bala-HF-Nle-NH₂ wherein QWAV-Bala-HF-Nle-NH₂ is SEQ ID NO: 9;
-monoamide-G-4-aminobenzoic acid-QWAGHFL-NH₂ wherein QWAGHFL-NH₂ is SEQ ID NO: 10;
-monoamide-G-4-aminobenzoic acid-LWAVGSFM-NH₂ wherein LWAVGSFM-NH₂ is SEQ ID NO: 11;
-monoamide-G-4-aminobenzoic acid-HWAVGHLM-NH₂ wherein HWAVGHLM-NH₂ is SEQ ID NO: 12;
-monoamide-G-4-aminobenzoic acid-LWAVGSFM-NH₂ wherein LWAVGSFM-NH₂ is SEQ ID NO: 11;
-monoamide-G-4-aminobenzoic acid-QWAVGHFM-NH₂ wherein QWAVGHFM-NH₂ is SEQ ID NO: 13;
-monoamide-Gly-3-aminobenzoic acid-BBN(7-14) wherein the BBN(7-14) sequence is SEQ ID NO: 1;
-monoamide-Gly-6-aminonaphthoic acid-BBN(7-14) wherein the BBN(7-14) sequence is SEQ ID NO: 1;
-monoamide-Gly-4-methylaminobenzoic acid-BBN(7-14) wherein the BBN(7-14) sequence is SEQ ID NO: 1;
m10d2a-Gly-4-aminobenzoic acid-BBN(7-14) wherein the BBN(7-14) sequence is SEQ ID NO: 1;
methylglycine-Ser-Cys(Acm)-Gly-Gly-4-aminobenzoic acid-BBN(7-14) wherein the BBN(7-14) sequence is SEQ ID NO: 1;
methylglycine-Ser-Cys(Acm)-Gly-Gly-3-amino-3-deoxycholic acid-BBN(7-14) wherein the BBN(7-14) sequence is SEQ ID NO: 1;
-monoamide-Gly-3-methoxy-4-aminobenzoic acid-BBN(7-14) wherein the BBN(7-14) sequence is SEQ ID NO: 1;
-monoamide-Gly-3-chloro-4-aminobenzoic acid-BBN(7-14) wherein the BBN(7-14) sequence is SEQ ID NO: 1;
-monoamide-Gly-3-methyl-4-aminobenzoic acid-BBN(7-14) wherein the BBN(7-14) sequence is SEQ ID NO: 1;
-monoamide-Gly-3-hydroxy-4-aminobenzoic acid-BBN(7-14) wherein the BBN(7-14) sequence is SEQ ID NO: 1;
-monoamide)₂-N,N′-Bis(2-aminoethyl)-succinamic acid-BBN(7-14) wherein the BBN(7-14) sequence is SEQ ID NO: 1;
-monoamide-G-4-aminobenzoic acid-QWAVGHFL-NH₂ wherein QWAVGHFL-NH₂ is SEQ ID NO: 22;
-monoamide-4-aminomethylbenzoic acid-L-1-Naphthylalanine-QWAVGHLM-NH₂ wherein QWAVGHLM-NH₂ is SEQ ID NO: 1;
-monoamide-G-4-aminobenzoic acid-QWAVGNMeHisLM-NH₂ wherein QWAVGNMeHisLM-NH₂ is SEQ ID NO: 15;
Gly-4-aminobenzoic acid-BBN(7-14) wherein the BBN(7-14) sequence is SEQ ID NO: 1; and
CyAazta-Gly-4-aminobenzoic acid-BBN(7-14) wherein the BBN(7-14) sequence is SEQ ID NO: 1.

178. The method of embodiment 171, wherein the labeled compound comprises DO3A-monoamide-Gly-4-aminobenzoic acid-BBN(7-14) wherein the BBN(7-14) sequence is SEQ ID NO: 1.

179. The method of embodiment 128, wherein the GRP receptor targeting peptide of said first dose is an agonist.

180. The method of embodiment 137, wherein the GRP receptor targeting peptide of said first dose is an agonist.

181. The method of embodiment 146,wherein the GRP receptor targeting peptide of said first dose is an agonist.

182. The method of embodiment 155, wherein the first GRP receptor targeting peptide is an agonist.

183. The method of embodiment 163, wherein the first GRP receptor targeting peptide is an agonist.

184. The method of embodiment 171, wherein the first GRP receptor targeting peptide is an agonist.

185. A method of treating bone or soft tissue metastases of prostate cancer comprising the step of administering to a subject a dose comprising:

an amount of radioactively labeled L70, N-[4-[[[[[4,7,10-Tris(carboxymethyl)-1,4,7,10-tetraazacyclododec-1-yl]acetyl]amino]acetyl]amino]benzoyl]-L-glutaminyl-L-tryptophyl-L-alanyl-L-valyl-glycyl-L-histidyl-L-leucyl-L-methioninamide.

186. The method of embodiment 185, wherein said radioactive label is ¹⁷⁷Lu.

187. The method of embodiment 185, wherein said dose is about 3-30 mCi/kg of ¹⁷⁷Lu-labeled L70, N-[4-[[[[[4,7,10-Tris(carboxymethyl)-1,4,7,10-tetraazacyclododec-1-yl]acetyl]amino]acetyl]amino]benzoyl]-L-glutaminyl-L-tryptophyl-L-alanyl-L-valyl-glycyl-L-histidyl-L-leucyl-L-methioninamide.

188. The method of embodiment 185 wherein said dose is administered intravenously.

189. The method of embodiment 185 wherein said tumor exhibits a reduction in aberrant vasculature.

190. The method of embodiment 185 wherein time to progression is increased by at least about 15%.

191. A method of treating hormone sensitive prostate cancer comprising the step of administering to a subject a dose comprising:

an amount of radioactively labeled L70, N-[4-[[[[[4,7,10-Tris(carboxymethyl)-1,4,7,10-tetraazacyclododec-1-yl]acetyl]amino]acetyl]amino]benzoyl]-L-glutaminyl-L-tryptophyl-L-alanyl-L-valyl-glycyl-L-histidyl-L-leucyl-L-methioninamide.

192. The method of embodiment 191 wherein said radioactive label is ¹⁷⁷Lu.

193. The method of embodiment 191 wherein said dose is about 3-30 mCi/kg of ¹⁷⁷Lu-labeled L70, N-[4-[[[[[4,7,10-Tris(carboxymethyl)-1,4,7,10-tetraazacyclododec-1 yl]acetyl]amino]acetyl]amino]benzoyl]-L-glutaminyl-L-tryptophyl-L-alanyl-L-valyl-glycyl-L-histidyl-L-leucyl-L-methioninamide.

194. The method of embodiment 191 wherein said dose is administered intravenously.

195. The method of embodiment 191 wherein said tumor exhibits a reduction in aberrant vasculature.

196. The method of embodiment 191 wherein time to progression is increased by at least about 15%.

197. A method of treating hormone refractory prostate cancer comprising the step of administering to a subject a dose comprising:

an amount of radioactively labeled L70, N-[4-[[[[[4,7,10-Tris(carboxymethyl)-1,4,7,10-tetraazacyclododec-1-yl]acetyl]amino]acetyl]amino]benzoyl]-L-glutaminyl-L-tryptophyl-L-alanyl-L-valyl-glycyl-L-histidyl-L-leucyl-L-methioninamide.

198. The method of embodiment 197 wherein said radioactive label is ¹⁷⁷Lu.

199. The method of embodiment 197 wherein said dose is about 3-30 mCi/kg of ¹⁷⁷Lu-labeled L70, N-[4-[[[[[4,7,10-Tris(carboxymethyl)-1,4,7,10-tetraazacyclododec-1yl]acetyl]amino]acetyl]amino]benzoyl]-L-glutaminyl-L-tryptophyl-L-alanyl-L-valyl-glycyl-L-histidyl-L-leucyl-L-methioninamide.

200. The method of embodiment 197 wherein said dose is administered intravenously.

201. A method of delaying progression of hormone sensitive prostate cancer comprising the step of administering to a subject a dose comprising:

an amount of radioactively labeled L70, N-[4-[[[[[4,7,10-Tris(carboxymethyl)-1,4,7,10-tetraazacyclododec-1-yl]acetyl]amino]acetyl]amino]benzoyl]-L-glutaminyl-L-tryptophyl-L-alanyl-L-valyl-glycyl-L-histidyl-L-leucyl-L-methioninamide, sufficient to cause a reduction in aberrant vasculature in a tumor or increased time to progression.

202. The method of embodiment 201 wherein said radioactive label is ¹⁷⁷Lu.

203. The method of embodiment 201 wherein said dose is about 3-30 mCi/kg of ¹⁷⁷Lu-labeled L70, N-[4-[[[[[4,7,10-Tris(carboxymethyl)-1,4,7,10-tetraazacyclododec-1-yl]acetyl]amino]acetyl]amino]benzoyl]-L-glutaminyl-L-tryptophyl-L-alanyl-L-valyl-glycyl-L-histidyl-L-leucyl-L-methioninamide.

204. The method of embodiment 201 wherein said dose is administered intravenously.

205. The method of embodiment 201 wherein said tumor exhibits a reduction in aberrant vasculature.

206. The method of embodiment 201 wherein time to progression is increased by at least about 15%.

207. The method of embodiment 201 wherein time to progression is increased by at least about 50%.

208. The method of embodiment 201 wherein time to progression is increased by about 100%.

209. A method of facilitating combination therapy in hormone sensitive prostate cancer comprising the step of administering to a subject a dose comprising:

an amount of radioactively labeled L70, N-[4-[[[[[4,7,10-Tris(carboxymethyl)-1,4,7,10-tetraazacyclododec-1-yl]acetyl]amino]acetyl]amino]benzoyl]-L-glutaminyl-L-tryptophyl-L-alanyl-L-valyl-glycyl-L-histidyl-L-leucyl-L-methioninamide, sufficient to cause a reduction in aberrant vasculature in a tumor or increased time to progression.

210. The method of embodiment 209 wherein said radioactive label is ¹⁷⁷Lu.

211. The method of embodiment 209 wherein said dose is about 3-30 mCi/kg of ¹⁷⁷Lu-labeled L 70, N-[4-[[[[[4,7,10-Tris(carboxymethyl)-1,4,7,10-tetraazacyclododec-1-yl]acetyl]amino]acetyl]amino]benzoyl]-L-glutaminyl-L-tryptophyl-L-alanyl-L-valyl-glycyl-L-histidyl-L-leucyl-L-methioninamide.

212. The method of embodiment 209 wherein said dose is administered intravenously.

213. The method of embodiment 209 wherein said tumor exhibits a reduction in aberrant vasculature.

214. The method of embodiment 209 wherein time to progression is increased by at least about 15%.

215. A method of decreasing aberrant vascular permeability in patients with hormone sensitive prostate cancer comprising the step of administering to a subject a dose comprising:

an amount of radioactively labeled L70, N-[4-[[[[[4,7,10-Tris(carboxymethyl)-1,4,7,10-tetraazacyclododec-1-yl]acetyl]amino]acetyl]amino]benzoyl]-L-glutaminyl-L-tryptophyl-L-alanyl-L-valyl-glycyl-L-histidyl-L-leucyl-L-methioninamide.

216. The method of embodiment 215 wherein said radioactive label is ¹⁷⁷Lu.

217. The method of embodiment 215 wherein said dose is about 3-30 mCi/kg of ¹⁷⁷Lu-labeled L70, N-[4-[[[[[4,7,10-Tris(carboxymethyl)-1,4,7,10-tetraazacyclododec-1-yl]acetyl]amino]acetyl]amino]benzoyl]-L-glutaminyl-L-tryptophyl-L-alanyl-L-valyl-glycyl-L-histidyl-L-leucyl-L-methioninamide.

218. The method of embodiment 215 wherein said dose is administered intravenously.

219. A method of examining the effect on the function of one of a variety of receptors when treated with one or more of a variety of drugs by looking at the effect such changes in function have on the GRP receptor with which they exhibit crosstalk.

220. A method of screening for the activity of the GRP receptor family in vitro using a radiolabeled agonist or antagonist of the GRP receptors, particularly radiolabeled L70 (AMBA), and most preferably L70 radiolabeled with Ga.

221. A method of imaging the activity of the GRP receptor family in vivo using a radiolabeled agonist or antagonist of the GRP receptors, particularly radiolabeled L70 (AMBA), and most preferably L70 radiolabeled with Ga.

222. A method of improving patient management of a patient who is undergoing treatment with a drug which targets a receptor that crosstalks with GRP-R comprising obtaining a series of images of the patient after administration of radiolabeled L70 to a patient prior to initial treatment (to obtain a baseline value), during treatment (to predict and monitor response, and detect when a shift in the tumor population may warrant a change of therapeutic), and at the end of or post treatment (to aid in determination of efficacy, next treatment steps and to predict or look for relapse) and assessing any change in GRP-R activity

We claim:

1-11. (canceled)

12. A method of monitoring the therapeutic effect of a drug targeted to a receptor that crosstalks with GRP-R comprising:
a) administering to the patient a composition comprising a compound of general formula:
M-N—O—P-G
wherein
M is an a metal chelator complexed with a metal radionuclide, or a moiety that contains a radiolabeled halogen such as ¹⁸F—, ¹²³I—, ¹²⁴I— or ¹³¹I—;
N is absent, an alpha amino acid, a non-alpha amino acid with a cyclic group or other linking group;
O is an alpha amino acid or a non-alpha amino acid with a cyclic group;
P is absent, an alpha amino acid, a non-alpha amino acid with a cyclic group, or other linking group; and
G is a GRP receptor targeting peptide,
wherein at least one of N, O or P is a non-alpha amino acid with a cyclic group;
b) imaging the patient;
c) assessing the activity of the GRP-R based on the image;
d) administering to the patient a drug which targets a receptor which crosstalks with GRP-R;
e) administering to the patient a composition comprising a compound of general formula:
M-N—O—P-G
wherein
M is an a metal chelator complexed with a metal radionuclide, or a moiety that contains a radiolabeled halogen such as ¹⁸F—, ¹²³I—, ¹²⁴I— or ¹³¹I—;
N is 0, an alpha amino acid, a non-alpha amino acid with a cyclic group or other linking group;
O is an alpha amino acid or a non-alpha amino acid with a cyclic group;
P is 0, an alpha amino acid, a non-alpha amino acid with a cyclic group, or other linking group; and
G is a GRP receptor targeting peptide,
wherein at least one of N, O or P is a non-alpha amino acid with a cyclic group;
f) imaging the patient;
g) assessing the activity of the GRP-R based on the image;
h) assessing the change in GRP-R activity after administration of the drug; and
i) assessing the therapeutic effect of the drug based on the change in GRP-R activity.

13. The method of claim 12, further comprising altering the treatment regimen based on the assessed therapeutic effect of the drug.

14. The method of claim 12, wherein steps a)-c) occur up to about 30 days before step d).

15. The method of claim 14, wherein steps a)-c) occur about 15 days before step d).

16. The method of claim 15, wherein steps a)-c) occur about 7 days before step d).

17. The method of claim 12, wherein steps e)-i) occur within about 15 days of step d).

18. The method of claim 17, wherein steps e)-i) occur within about 7 days of step d).

19. The method of claim 12, wherein the method is repeated several times during the treatment process.

20. The method of claim 12, wherein the receptor that crosstalks with GRP-R is selected from the group consisting of the estrogen receptor and receptor tyrosine kinases (RTKs).

21. The method of claim 20, wherein the RTK is selected from the group consisting of EGFR, the Src family, HER2/ErbB3, Bcr-Abl, SCF, KIT, PDGF, VEGF-R1,2,3, FLT3, Ras, Raf, CSF-1R, and RET.

22. The method of claim 12, wherein the drug is selected from the group consisting of Exemestane, Lapatinib, Dasatinib, Gefitinib, Imatinib, Erlotinib, Sorafenib, Sunitinib, Anastrozole, Bortezomib, Tamoxifen, and β2-estradiol.

23. The method of claim 12, wherein the compound of formula M-N—O—P-G is L70 of formula: 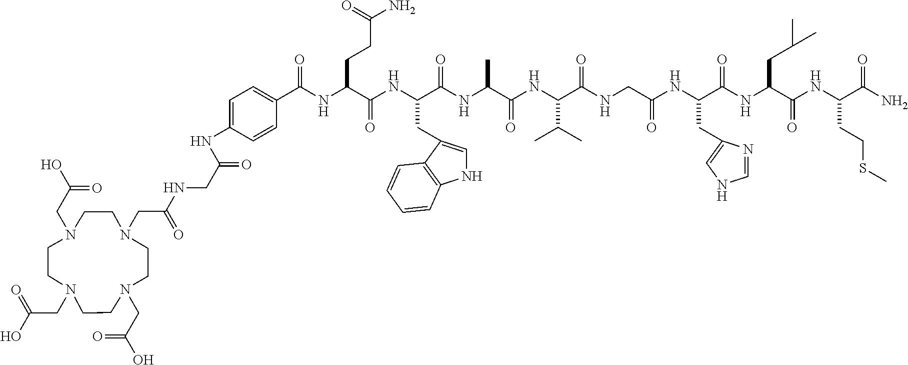 [SMILES: CSCC[C@H](NC(=O)[C@H](CC(C)C)NC(=O)[C@H](Cc1c[nH]cn1)NC(=O)CNC(=O)[C@@H](NC(=O)[C@H](C)NC(=O)[C@H](Cc1c[nH]c2ccccc12)NC(=O)[C@H](CCC(N)=O)NC(=O)c1ccc(NC(=O)CNC(=O)CN2CCN(CC(=O)O)CCN(CC(=O)O)CCN(CC(=O)O)CC2)cc1)C(C)C)C(N)=O]
complexed with a diagnostic radionuclide.

24. The method of claim 23 wherein L70 is complexed with a radionuclide detectable by PET.

25. The method of claim 23, wherein L70 is complexed with a radionuclide detectable by SPECT.

26. The method of claim 24, wherein L70 is complexed with ⁶⁸Ga.

27. The method of any one of claims 23 or 26, wherein the composition further comprises a buffer and selenomethionine.

28. The method of claim 27, wherein the buffer comprises sodium acetate.

29. The method of claim 27, wherein the composition further comprises ascorbic acid, EDTA and saline.

30. The method of claim 12, wherein the patient is imaged using PET.

31. The method of claim 12, wherein the patient is imaged using SPECT.

32. The method of claim 12, further comprising administering a second GRP receptor targeting peptide, optionally conjugated to a linker and/or a chelator, to occupy GRP receptor binding sites in non-target tissue prior to administering the composition in steps a) or e), or in steps a) and e).

33. The method of claim 12, further comprising, in steps a) or e), or in steps a) and e), administering a combination of:
a) a first GRP receptor targeting peptide optionally conjugated to a linker and/or a chelator, to occupy GRP receptor binding sites in non-target tissue; and
b) the radiolabeled composition.


SEQUENCE LISTING (36 sequences)

SEQ ID NO: 1 - protein, 8 aa, Artificial
  Description of Artificial Sequence  This peptide is known as bombesin (i.e., BBN (7-14)) and binds to gastrin releasing peptide (GRP) receptors.
QWAVGHLM

SEQ ID NO: 2 - protein, 8 aa, Artificial
  Description of Artificial Sequence  GRP Receptor Targeting Peptide
EWAVGHLM

SEQ ID NO: 3 - protein, 13 aa, Artificial
  Description of Artificial Sequence  GRP Receptor Targeting Peptide
QRLGNQWAVGHLM

SEQ ID NO: 4 - protein, 13 aa, Artificial
  Description of Artificial Sequence  GRP Receptor Targeting Peptide
QRYGNQWAVGHLM

SEQ ID NO: 5 - protein, 13 aa, Artificial
  Description of Artificial Sequence  GRP Receptor Targeting Peptide
QKYGNQWAVGHLM

SEQ ID NO: 6 - protein, 7 aa, Artificial
  Description of Artificial Sequence  GRP Receptor Targeting Peptide
QWAVGHL

SEQ ID NO: 7 - protein, 7 aa, Artificial
  Description of Artificial Sequence  GRP Receptor Targeting Peptide
QWSVHLM

SEQ ID NO: 8 - protein, 8 aa, Artificial
  Description of Artificial Sequence  GRP Receptor Targeting Peptide
QWAVGHLL

SEQ ID NO: 9 - protein, 8 aa, Artificial
  Description of Artificial Sequence  GRP Receptor Targeting Peptide
QWAVAHFL

SEQ ID NO: 10 - protein, 7 aa, Artificial
  Description of Artificial Sequence  GRP Receptor Targeting Peptide
QWAGHFL

SEQ ID NO: 11 - protein, 8 aa, Artificial
  Description of Artificial Sequence  GRP Receptor Targeting Peptide
QWAVGHFL

SEQ ID NO: 12 - protein, 8 aa, Artificial
  Description of Artificial Sequence  GRP Receptor Targeting Peptide
LWAVGSFM

SEQ ID NO: 13 - protein, 8 aa, Artificial
  Description of Artificial Sequence  GRP Receptor Targeting Peptide
HWAVGHLM

SEQ ID NO: 14 - protein, 8 aa, Artificial
  Description of Artificial Sequence  GRP Receptor Targeting Peptide
QWAVGHFM

SEQ ID NO: 15 - protein, 7 aa, Artificial
  Description of Artificial Sequence  GRP Receptor Targeting Peptide
QWAVHLM

SEQ ID NO: 16 - protein, 9 aa, Artificial
  Description of Artificial Sequence  GRP Receptor Targeting Peptide
QWAVGNHLM

SEQ ID NO: 17 - protein, 8 aa, Artificial
  Description of Artificial Sequence  GRP Receptor Targeting Peptide
LWATGHFM

SEQ ID NO: 18 - protein, 11 aa, Artificial
  Description of Artificial Sequence  GRP Receptor Targeting Peptide
LGNQWAVGHLM

SEQ ID NO: 19 - protein, 7 aa, Artificial
  Description of Artificial Sequence  GRP Receptor Targeting Peptide
WAVGHLM

SEQ ID NO: 20 - protein, 10 aa, Artificial
  Description of Artificial Sequence  GRP Receptor Targeting Peptide
GNLWATGHFM

SEQ ID NO: 21 - protein, 9 aa, Artificial
  Description of Artificial Sequence  GRP Receptor Targeting Peptide
FQWAVAHFL

SEQ ID NO: 22 - protein, 9 aa, Artificial
  Description of Artificial Sequence  GRP Receptor Targeting Peptide
GQWAVGHLM

SEQ ID NO: 23 - protein, 14 aa, Artificial
  Description of Artificial Sequence  GRP Receptor Targeting Peptide
EQRYGNQWAVGHLM

SEQ ID NO: 24 - protein, 8 aa, Artificial
  Description of Artificial Sequence  GRP Receptor Targeting Peptide
QWAVGHLM

SEQ ID NO: 25 - protein, 7 aa, Artificial
  Description of Artificial Sequence  GRP Receptor Targeting Peptide
WAVGHLL

SEQ ID NO: 26 - protein, 7 aa, Artificial
  Description of Artificial Sequence  GRP Receptor Targeting Peptide
LWAGHFM

SEQ ID NO: 27 - protein, 8 aa, Artificial
  Description of Artificial Sequence  GRP Receptor Targeting Peptide
LWATGSFM

SEQ ID NO: 28 - protein, 8 aa, Artificial
  Description of Artificial Sequence  GRP Receptor Targeting Peptide
QWAVGHLM

SEQ ID NO: 29 - protein, 8 aa, Artificial
  Description of Artificial Sequence  GRP Receptor Targeting Peptide
QWAVGHLM

SEQ ID NO: 30 - protein, 8 aa, Artificial
  Description of Artificial Sequence  GRP Receptor Targeting Peptide
QWAVGHLM

SEQ ID NO: 31 - protein, 9 aa, Artificial
  Description of Artificial Sequence  GRP Receptor Targeting Peptide
QWNAVGHLM

SEQ ID NO: 32 - protein, 8 aa, Artificial
  Description of Artificial Sequence  GRP Receptor Targeting Peptide
QWAVSHLM

SEQ ID NO: 33 - protein, 9 aa, Artificial
  Description of Artificial Sequence  GRP Receptor Targeting Peptide
QWAVGNHLM

SEQ ID NO: 34 - protein, 8 aa, Artificial
  Description of Artificial Sequence  GRP Receptor Targeting Peptide
QWAVGHLM

SEQ ID NO: 35 - protein, 9 aa, Artificial
  Description of Artificial Sequence  GRP Receptor Targeting Peptide
QWAVGHNLM

SEQ ID NO: 36 - protein, 9 aa, Artificial
  Description of Artificial Sequence  GRP Receptor Targeting Peptide
FQWAVAHFL